(19) United States
(12) Patent Application Publication
(10) Pub. No.: US 2025/0282742 A1
(43) Pub. Date: Sep. 11, 2025

(54) BENZOFURAN DERIVATIVES FOR TARGETING AUTOPHAGY

(71) Applicant: Automera PTE. LTD, Singapore (SG)

(72) Inventors: Shuyi Pearly NG, Singapore (SG); Mohammed Mahmood AHMED, Singapore (SG); Shawn Siang HOON, Singapore (SG); Kenrick An Fu YAP, Singapore (SG)

(21) Appl. No.: 19/073,880

(22) Filed: Mar. 7, 2025

Related U.S. Application Data
(60) Provisional application No. 63/563,122, filed on Mar. 8, 2024.

(51) Int. Cl. C07D 307/81; A61K 31/343; A61K 31/443; A61K 31/4525; C07D 307/82; C07D 405/06

(52) U.S. Cl. CPC C07D 307/81; A61K 31/343; A61K 31/443; A61K 31/4525; C07D 307/82; C07D 405/06

(57) ABSTRACT

Provided herein are compounds of formula (I), (II), and (III), or pharmaceutically acceptable salts, stereoisomers, or deuterated forms thereof, wherein X¹, X², X³, R¹, R², a, b, L⁰, L¹, L², and A are defined herein. Also provided herein are pharmaceutical compositions comprising a compound of formula (I), (II), or (III), or pharmaceutically acceptable salt, a stereoisomer, or deuterated form thereof, and methods of using a compound of formula (I), (II), or (III), or pharmaceutically acceptable salt, a stereoisomer, or deuterated form thereof, e.g., in the treatment of a disease or disorder by modulating autophagic degradation and/or p62 activity.

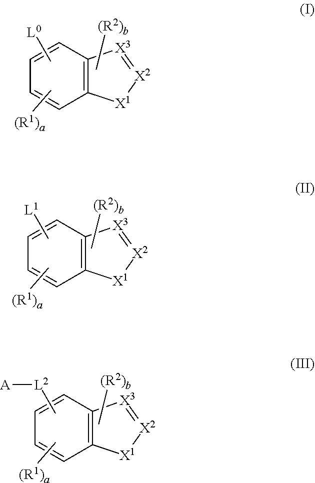

### CROSS-REFERENCE TO RELATED APPLICATIONS

This application claims priority to U.S. Provisional Patent Application No. 63/563,122, filed on Mar. 8, 2024, the content of which is hereby incorporated by reference in its entirety for all purposes.

### BACKGROUND

Autophagy is a major degradation pathway for maintaining cellular homeostasis. Autophagy eliminates unnecessary, aged, dysfunctional, or damaged intracellular components through lysosome-mediated degradation. The autophagy process can degrade bulky cellular cargoes (e.g., proteins aggregates, intracellular pathogens) that cannot be degraded by other processes, such as the ubiquitin-proteasome system (UPS).

During autophagy, cytoplasmic contents are delivered into the lysosomal system by double-membraned organelles called autophagosomes. Specifically, cytoplasmic material is sequestered into autophagosomes, which subsequently fuse with lysosomes where degradation occurs via the action of acidic lysosomal hydrolases. Autophagy helps to keep cells healthy, and dysregulation of this process can contribute to a wide range of diseases, including cancer, inflammation, neurodegeneration, and infectious diseases.

Sequestosome-1, also known as ubiquitin-binding protein p62 (SQSTM1 or p62, hereinafter “p62”), is a key autophagy receptor for targeted degradation. p62 operates as an autophagy adaptor that brings ubiquitinated substrates (e.g., damaged proteins) into contact with autophagosomes in preparation for autophagy. In addition to autophagic degradation, p62 also plays an important role in the UPS, cellular signaling, metabolism, and apoptosis.

There is a need to develop therapeutics that target p62 and modulate autophagy.

### SUMMARY

In one aspect, the present disclosure provides a compound of formula (I):

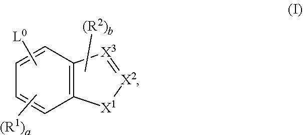

or a pharmaceutically acceptable salt, stereoisomer, or deuterated form thereof, wherein:
L⁰ is —C₁₋₆ alkylene-NR^{A}R^{B}, —C₁₋₆ alkylene-NR^{A}—C₁₋₆ alkylene-R^{B}, —C₁₋₆ alkylene-NR^{A}—C₃₋₈ cycloalkylene-R^{B}, —C₁₋₆ alkylene-NR^{A}C(O)NR^{A}R^{B}, —C₁₋₆ alkylene-NR^{A}C(O)—R^{B}, —C₁₋₆ alkylene-O—C₁₋₆ alkylene-NR^{A}R^{B}, —C₁₋₆ alkylene-O—C₁₋₆ alkylene-C(O)NR^{A}R^{B}, —C₁₋₆ alkylene-O—C₁₋₆ alkylene-NR^{A}C(O)—R^{B}, —C₁₋₆ alkylene-heterocyclylene-O—C₁₋₆ alkyl, —C₃₋₈ cycloalkylene-NR^{A}R^{B}, —C(O)NR^{A}R^{B}, —O—C₁₋₆ alkylene-C(O)NR^{A}R^{B}, —O—C₁₋₆ alkylene-NR^{A}R^{B}, —NR^{A}C(O)R^{B}—NR^{A}C(O)NR^{A}R^{B}, or —NR^{A}C(O)NR^{A}—C₁₋₆ alkylene-R^{B}, wherein the alkylene is optionally substituted with —OH or halogen;
each R¹ is independently C₁₋₆ alkyl, C₃-C₈ cycloalkyl, or halogen;
each R² is independently C₁₋₆ alkyl, C₃-C₈ cycloalkyl, or halogen;
X¹ is O or S;
one of X² or X³ is C-Q-R³ and the other is CH, C, or N as permitted by valency;
Q is absent, C₁₋₆ alkylene, —C₁₋₆ alkylene-C(O)—, C₃₋₈ cycloalkylene, —C₃₋₈ cycloalkylene-C(O)—, —C(O)—, or —S(O)₂—;
R³ is carbocycle, heterocycle, aryl, heteroaryl, or —NR^{X}R^{Y}, wherein the carbocycle, heterocycle, aryl, and heteroaryl are each optionally substituted with 1, 2, or 3 R⁴;
each R⁴ is independently halogen, —OH, C₁₋₆ alkyl, C₁₋₆ alkoxy, —C(O)—NR^{X}R^{Y}, or —C(O)O—(C₁₋₆ alkyl);
each R^{A} is independently H, C₁₋₆ alkyl, C₁₋₆ haloalkyl, C₃₋₈ cycloalkyl, C₃₋₈ hydroxycycloalkyl, aryl, or heterocycle;
each R^{B} is independently C₁₋₆ alkyl, C₁₋₆ hydroxyalkyl, C₁₋₆ alkoxy, C₃₋₈ hydroxycycloalkyl, C₁₋₆ alkylene-NH₂, or C₁₋₆ alkylene-SH;
R^{X} is H, C₁₋₆ alkyl, C₁₋₆ haloalkyl, or C₁₋₆ hydroxyalkyl;
R^{Y} is C₁₋₆ alkyl, C₃₋₈ cycloalkyl, C₃₋₈ hydroxycycloalkyl, aryl, or heterocycle;
a is an integer of 0-3; and
b is an integer of 0 or 1.

In another aspect, the present disclosure provides a compound of formula (II)

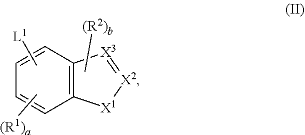

or a pharmaceutically acceptable salt, stereoisomer, or deuterated form thereof, wherein:
L¹ is C₂-C₅₀ alkylene-R⁵, C₂-C₂₅ alkenylene-R⁵, C₂-C₂₅ alkynylene-R⁵, wherein 1-25 methylene groups of L¹ are optionally and independently replaced by —N(H)—, —N(C₁-C₆ alkyl)-, —N(C₃-C₈ cycloalkyl)-, —O—, —C(O)—, —C(O)O—, —S—, —S(O)—, —S(O)₂—, —S(O)₂N(C₁-C₆ alkyl)-, —S(O)₂N(C₃-C₈ cycloalkyl)-, —N(H)C(O)—, —N(C₁-C₆ alkyl)C(O)—, —N(C₃-C₈ cycloalkyl)C(O)—, —N(H)C(O)N(H)—, —N(C₁-C₆ alkyl)C(O)N(H)—, —N(H)C(O)N(C₁-C₆ alkyl)-, —N(C₁-C₆ alkyl)C(O)N(C₁-C₆ alkyl)-, —C(O)N(H)—, —C(O)N(C₁-C₆ alkyl)-, —C(O)N(C₃-C₈ cycloalkyl)-, arylene, heteroarylene, heterocyclylene, C₃-C₈ cycloalkylene, or C₃-C₈ cycloalkenylene, wherein the alkylene, alkenylene, alkynylene, alkyl, arylene, heteroarylene, and heterocyclylene are each optionally and independently substituted with 1, 2, or 3 R^{Z}, wherein each R^{Z} is independently —OH, C₁₋₆ alkyl, or halogen;
each R¹ is independently C₁₋₆ alkyl, C₃-C₈ cycloalkyl, or halogen;
each R² is independently C₁₋₆ alkyl, C₃-C₈ cycloalkyl, or halogen;
X¹ is O or S;
one of X² or X³ is C-Q-R³ and the other is CH, C, or N as permitted by valency;
Q is absent, C₁₋₆ alkylene, —C₁₋₆ alkylene-C(O)—, C₃₋₈ cycloalkylene, —C₃₋₈ cycloalkylene-C(O)—, —C(O)—, or —S(O)₂—;
R³ is carbocycle, heterocycle, aryl, heteroaryl, or —NR^{X}R^{Y}, wherein the carbocycle, heterocycle, aryl, and heteroaryl are each optionally substituted with 1, 2, or 3 R⁴;
each R⁴ is independently halogen, —OH, C₁₋₆ alkyl, C₁₋₆ alkoxy, —C(O)—NR^{X}R^{Y}, or —C(O)O—(C₁₋₆ alkyl);
each R⁵ is independently a bond, a leaving group, a protecting group, H, alkynyl, aryl, or heteroaryl;
R^{X} is H, C₁₋₆ alkyl, C₁₋₆ haloalkyl, or C₁₋₆ hydroxyalkyl;
R^{Y} is C₁₋₆ alkyl, C₃₋₈ cycloalkyl, C₃₋₈ hydroxycycloalkyl, aryl, or heterocycle;
a is an integer of 0-3; and
b is an integer of 0 or 1.

In a further aspect, the present disclosure provides a compound of formula (III)

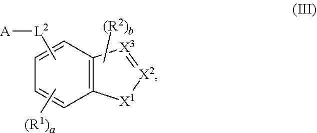

or a pharmaceutically acceptable salt, stereoisomer, or deuterated form thereof, wherein:
A is a ligand that binds to a protein, a protein aggregate, a protein complex, or a lipid;
L² is C₂-C₅₀ alkylene, C₂-C₂₅ alkenylene, C₂-C₂₅ alkynylene, or —(C₂-C₅₀ alkylene)-arylene, wherein 1-25 methylene groups of L² are optionally and independently replaced by —N(H)—, —N(C₁-C₆ alkyl)-, —N(C₃-C₈ cycloalkyl)-, —O—, —C(O)—, —C(O)O—, —S—, —S(O)—, —S(O)₂—, —S(O)₂N(C₁-C₆ alkyl)-, —S(O)₂N(C₃-C₈ cycloalkyl)-, —N(H)C(O)—, —N(C₁-C₆ alkyl)C(O)—, —N(C₃-C₈ cycloalkyl)C(O)—, —N(H)C(O)N(H)—, —N(C₁-C₆ alkyl)C(O)N(H)—, —N(H)C(O)N(C₁-C₆ alkyl)-, —N(C₁-C₆ alkyl)C(O)N(C₁-C₆ alkyl)-, —C(O)N(H)—, —C(O)N(C₁-C₆ alkyl)-, —C(O)N(C₃-C₈ cycloalkyl)-, arylene, heteroarylene, heterocyclylene, C₃-C₈ cycloalkylene, or C₃-C₈ cycloalkenylene, wherein the alkylene, alkenylene, alkynylene, alkyl, arylene, heteroarylene, and heterocyclylene are each optionally and independently substituted with 1, 2, or 3 R^{Z}, wherein each R^{Z} is independently OH, C₁₋₆ alkyl, or halogen;
each R¹ is independently C₁₋₆ alkyl, C₃-C₈ cycloalkyl, or halogen;
each R² is independently C₁₋₆ alkyl, C₃-C₈ cycloalkyl, or halogen;
X¹ is O or S;
one of X² or X³ is C-Q-R³ and the other is CH, C, or N as permitted by valency;
Q is absent, C₁₋₆ alkylene, —C₁₋₆ alkylene-C(O)—, C₃₋₈ cycloalkylene, —C₃₋₈ cycloalkylene-C(O)—, —C(O)—, or —S(O)₂—;
R³ is carbocycle, heterocycle, aryl, heteroaryl, or —NR^{X}R^{Y}, wherein the carbocycle, heterocycle, aryl, and heteroaryl are each optionally substituted with 1, 2, or 3 R⁴;
each R⁴ is independently halogen, —OH, C₁₋₆ alkyl, C₁₋₆ alkoxy, —C(O)—NR^{X}R^{Y}, or —C(O)O—(C₁₋₆ alkyl);
R^{X} is H, C₁₋₆ alkyl, C₁₋₆ haloalkyl, or C₁₋₆ hydroxyalkyl;
R^{Y} is C₁₋₆ alkyl, C₃₋₈ cycloalkyl, C₃₋₈ hydroxycycloalkyl, aryl, or heterocycle;
a is an integer of 0-3; and
b is an integer of 0 or 1.

### BRIEF DESCRIPTION OF THE DRAWINGS

FIG. 1A illustrates general synthetic route A for preparing compounds of the present disclosure.

FIG. 1B illustrates general synthetic route B for preparing compounds of the present disclosure.

FIG. 1C illustrates general synthetic route C for preparing compounds of the present disclosure.

FIG. 1D illustrates general synthetic route D for preparing compounds of the present disclosure.

FIG. 1E illustrates general synthetic route E for preparing compounds of the present disclosure.

FIG. 1F illustrates general synthetic route F for preparing compounds of the present disclosure.

FIG. 1G illustrates general synthetic route G for preparing compounds of the present disclosure.

FIG. 1H illustrates general synthetic route H for preparing compounds of the present disclosure.

FIG. 1I illustrates general synthetic route I for preparing compounds of the present disclosure.

FIG. 1J illustrates general synthetic route J for preparing compounds of the present disclosure.

FIG. 1K illustrates general synthetic route K for preparing compounds of the present disclosure.

FIG. 1L illustrates general synthetic route L for preparing compounds of the present disclosure.

FIG. 1M illustrates general synthetic route M for preparing compounds of the present disclosure.

FIGS. 2A-C shows representative western blots for p62 oligomerization of compounds 2 (FIG. 2A), 15 (FIG. 2B), and 49 (FIG. 2C).

FIGS. 3A-C shows representative Western blots for LC3 lipidation of compounds 2 (FIG. 3A), 37 (FIG. 3B), and 43 (FIG. 3C).

FIGS. 4A-B shows representative Western blots for protein degradation by compound 101 (FIG. 4A) and compound 100 (FIG. 4B).

FIG. 5 shows representative Western blots for protein degradation by compound 104.

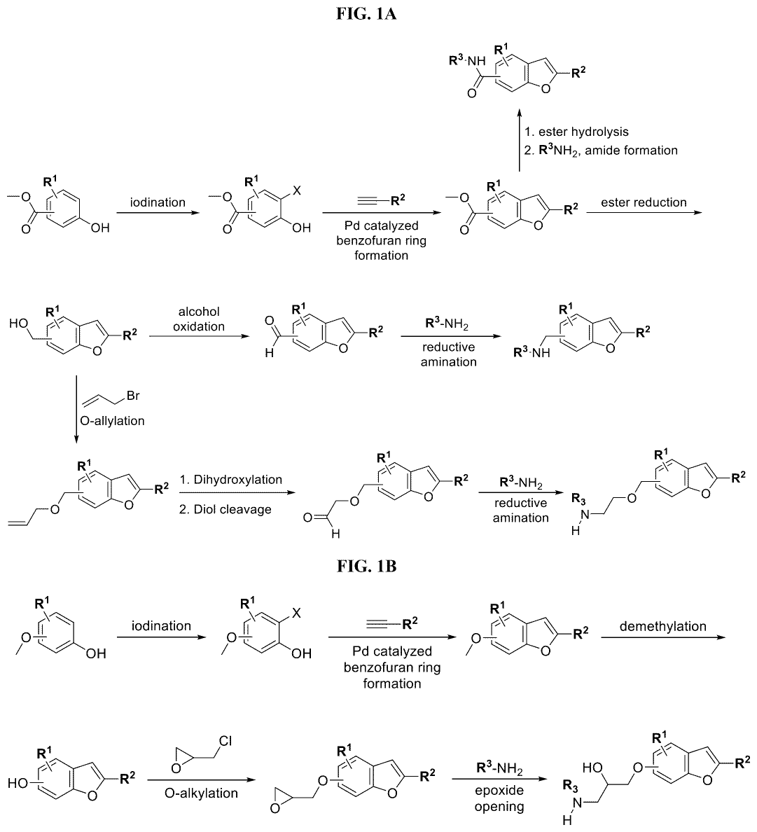

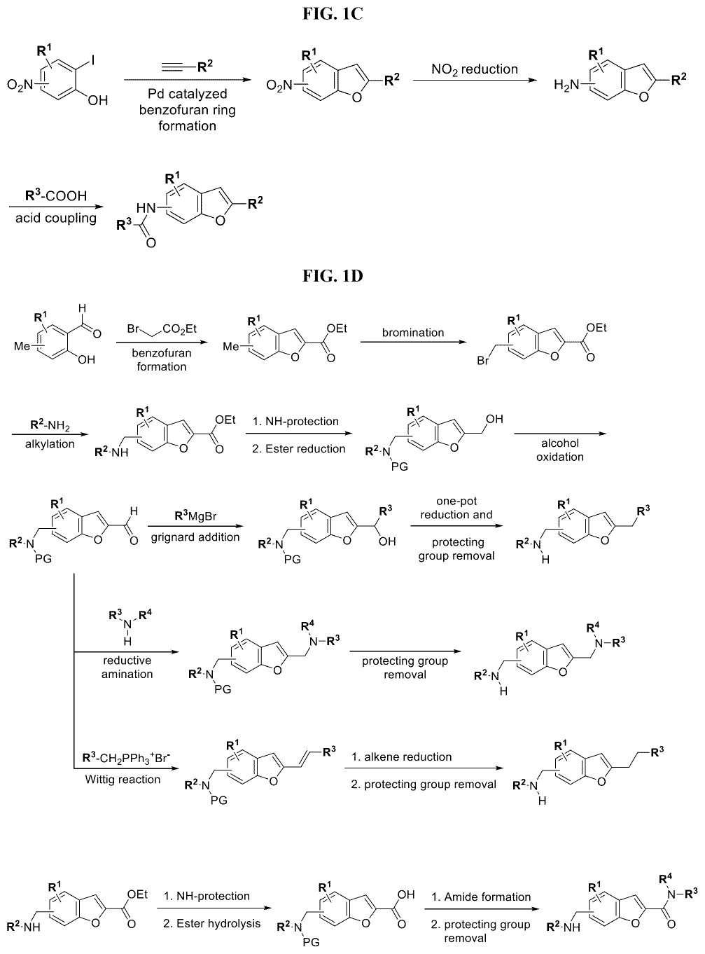

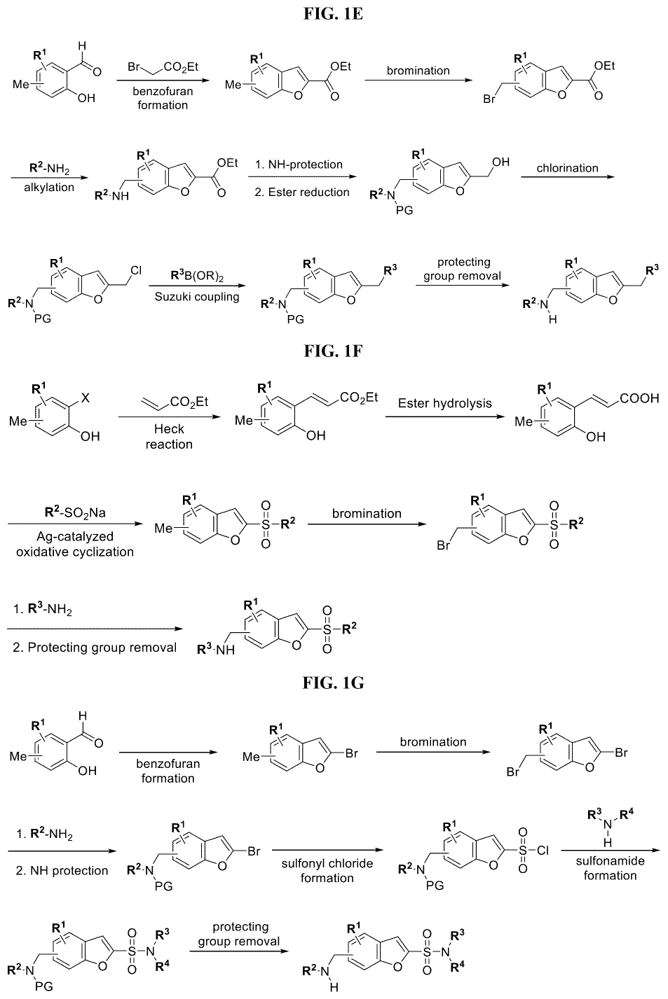

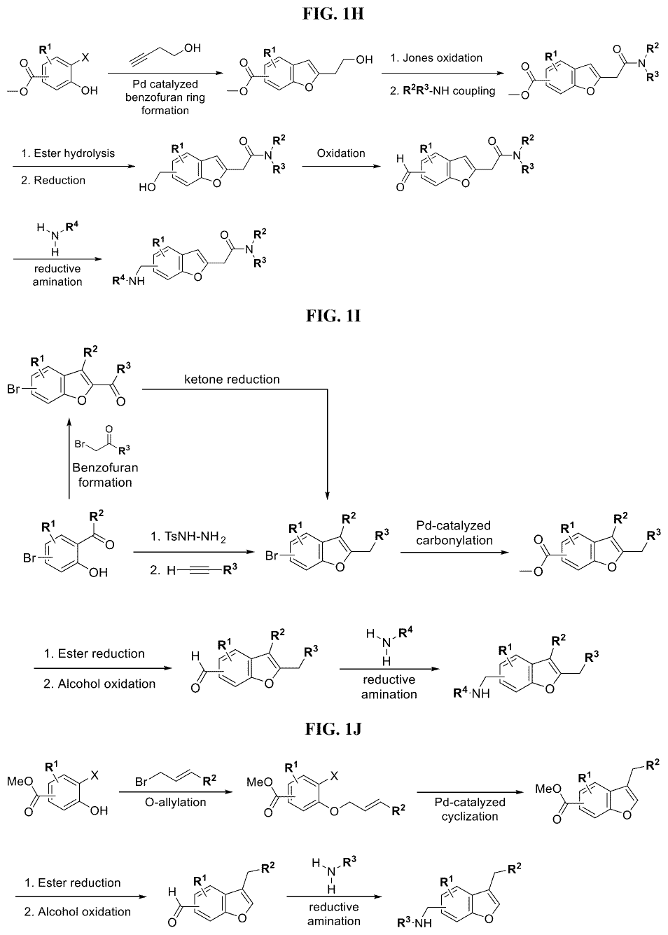

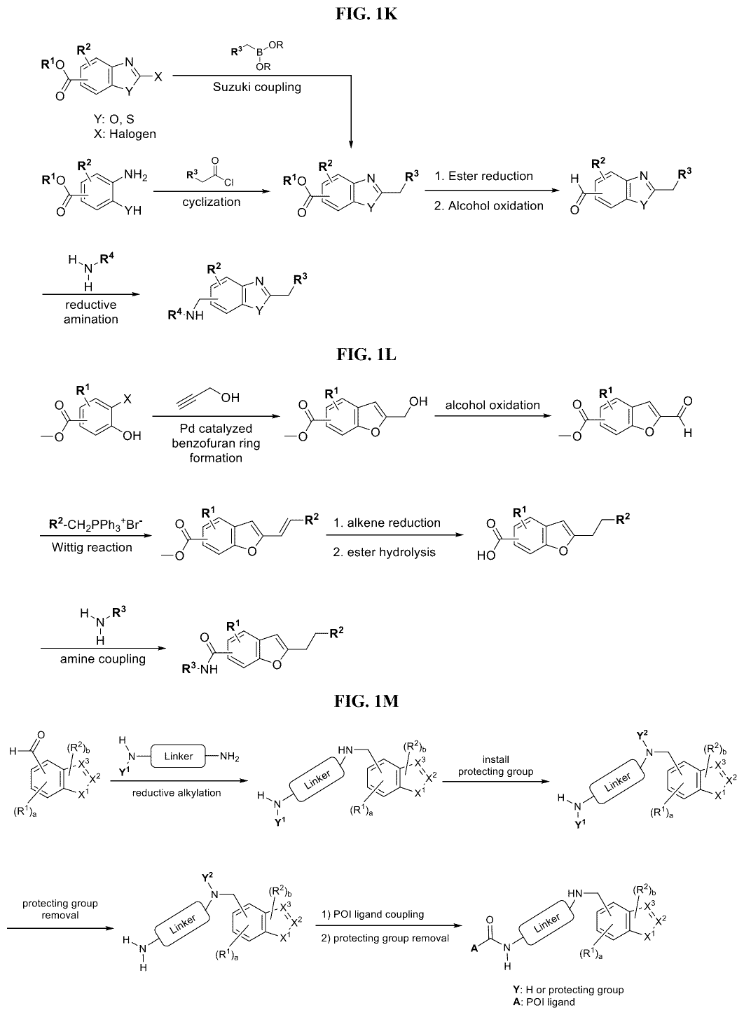

### DETAILED DESCRIPTION

Targeted Protein degradation (TPD) has emerged as a promising modality in drug discovery, typically involving heterobifunctional molecules that combine a target binder with a degradation-inducing moiety (Békés, Miklós, David R. Langley, and Craig M. Crews. 2022. “PROTAC Targeted Protein Degraders: The Past Is Prologue.” Nature Reviews. Drug Discovery 21 (3): 181-200, incorporated herein by reference in its entirety). This approach is particularly attractive for targeting proteins previously considered undruggable. However, current TPD technologies, such as PROteolysis-TArgeting Chimeras (PROTACs), are primarily limited to inducing ubiquitination of target substrates for degradation. Despite its potential, PROTAC technology faces limitations due to challenges in forming substrate-PROTAC-E3 ligase complexes, restricting its application to a limited set of targets and E3 ligases.

Autophagy is a vital cellular process that manages the degradation and recycling of cellular components (Lamark, Trond, and Terje Johansen. 2021. “Mechanisms of Selective Autophagy.” Annual Review of Cell and Developmental Biology 37 (October): 143-69; Aman, Yahyah, Tomas Schmauck-Medina, Malene Hansen, Richard I. Morimoto, Anna Katharina Simon, Ivana Bjedov, Konstantinos Palikaras, et al. 2021. “Autophagy in Healthy Aging and Disease.” Nature Aging 1 (8): 634-50; each incorporated herein by reference in their entirety). It plays a crucial role in maintaining cellular homeostasis by removing damaged or dysfunctional organelles and proteins. The process of autophagy involves the formation of autophagosomes, which engulf cellular debris and fuse with lysosomes for degradation. This mechanism is essential for cellular health, and its dysfunction is associated with various diseases, including neurodegenerative disorders, cancers, and metabolic diseases. Autophagy can be broadly classified into macroautophagy, microautophagy, and chaperone-mediated autophagy, with each type serving specific functions in the cell.

Autophagy plays a dual role in disease pathology. While it can prevent the accumulation of toxic protein aggregates and damaged organelles, its dysregulation can also contribute to disease progression. In neurodegenerative diseases such as Alzheimer's, Parkinson's, and Huntington's disease, impaired autophagy leads to the accumulation of misfolded proteins, exacerbating neurodegeneration. In cancer, autophagy can have both tumor-suppressive and tumor-promoting roles, depending on the context. Enhancing or inhibiting autophagy has been explored as a therapeutic strategy in various diseases. For instance, autophagy inducers are being investigated for their potential to clear protein aggregates in neurodegenerative diseases, while autophagy inhibitors are being explored in certain cancers (Maiuri, Maria Chiara, and Guido Kroemer. 2019. “Therapeutic Modulation of Autophagy: Which Disease Comes First?” Cell Death and Differentiation 26 (4): 680-89, incorporated herein by reference in its entirety).

p62 is a multifunctional protein that plays a significant role in autophagy, particularly in selective autophagy (Kumar, Anita V., Joslyn Mills, and Louis R. Lapierre. 2022. “Selective Autophagy Receptor P62/SQSTM1, a Pivotal Player in Stress and Aging.” Frontiers in Cell and Developmental Biology 10 (February): 793328, incorporated herein by reference in its entirety). It acts as a link between LC3 (a protein associated with autophagosomes) and ubiquitinated substrates. p62 binds to ubiquitin-tagged proteins and aggregates, delivering them to autophagosomes for degradation. It is also involved in the formation of protein aggregates known as aggresomes, which are targeted for autophagic degradation. The regulation of p62 and its interaction with other autophagy-related proteins are critical for the efficient execution of selective autophagy.

p62 is a key autophagy adaptor involved in the autophagic degradation of ubiquitinated substrates. p62 also interacts with ATG8 proteins that are found on the surface of developing autophagosomes. As such, p62 enables selective degradation by directing ubiquitinated substrates to the growing autophagosomes. Oligomerization of individual p62 units (i.e., p62 oligomer) has been shown to provide a stronger interaction with autophagosome. In addition to autophagic degradation, p62 influences other cellular pathways and is associated with pathological conditions including neurodegenerative diseases and cancer.

The N-degron pathway recognizes specific N-terminal amino acids (N-degrons) of proteins for degradation (Varshavsky, Alexander. 2019. “N-Degron and C-Degron Pathways of Protein Degradation.” Proceedings of the National Academy of Sciences of the United States of America 116 (2): 358-66, incorporated herein by reference in its entirety). The Arginylation branch of this pathway utilizes Arg, Lys, His (type 1), and Phe, Tyr, Trp, Leu, Ile (type 2) as N-degrons. Recent discoveries have shown that the Arg/N-degron pathway mediates not only ubiquitylation-dependent proteasomal clearance but also macroautophagic protein degradation. In this process, p62/SQSTM1 acts as an N-recognin, binding type-1 and type-2 N-degrons via its ZZ domain, activating p62 into an autophagy-compatible form for efficient autophagosome biogenesis (Kwon, Do Hoon, Ok Hyun Park, Leehyeon Kim, Yang Ouk Jung, Yeonkyoung Park, Hyeongseop Jeong, Jaekyung Hyun, Yoon Ki Kim, and Hyun Kyu Song. 2018. “Insights into Degradation Mechanism of N-End Rule Substrates by P62/SQSTM1 Autophagy Adapter.” Nature Communications 9 (1): 3291; Cha-Molstad, Hyunjoo, Ji Eun Yu, Zhiwei Feng, Su Hyun Lee, Jung Gi Kim, Peng Yang, Bitnara Han, et al. 2017. “P62/SQSTM1/Sequestosome-1 Is an N-Recognin of the N-End Rule Pathway Which Modulates Autophagosome Biogenesis.” Nature Communications 8 (1): 1-17; each incorporated herein by reference in their entirety).

p62 (SQSTM1) and NBR1 are key autophagy receptors that mediate the selective degradation of ubiquitinated proteins by bridging cargo to the autophagic machinery. Both proteins share several conserved domains critical to their function, including the ZZ-type zinc finger domain (ZZ domain), a PB1 domain for oligomerization, a UBA domain for binding polyubiquitinated substrates, and an LC3-interacting region (LIR) essential for autophagosome recruitment.

A defining feature of p62 and NBR1 is their ability to bind LC3, a core autophagy component embedded in the autophagosome membrane. This interaction, driven by their LIR motifs, is crucial for targeting cargo to autophagosomes for subsequent lysosomal degradation. Oligomerization through the PB1 domain promotes the clustering of cargo, enhancing both LC3 binding and autophagosome formation. The ZZ domain further stabilizes this process by indirectly facilitating interactions with ubiquitinated substrates and upstream signalling proteins involved in autophagy initiation.

Given the high degree of conservation between p62 and NBR1, the designed warheads could potentially interact with both receptors, offering a broad mechanism for modulating selective autophagy.

This disclosure presents compositions and methods for the manipulation of the intrinsic autophagic pathway for the selective degradation of pathogenic proteins. The disclosure comprises novel heterobifunctional compounds designed to engage the autophagy adaptor protein p62/SQSTM1, thereby triggering its activation and subsequent assembly of autophagosomes. These chimeric molecules are composed of a targeting ligand (also referred to as a “protein binding component” or “PBC”), which exhibits high-affinity binding to designated pathogenic proteins, conjoined via a designed, flexible linker to a p62 activating moiety (also referred to as a “warhead”). This bifunctional architecture enables the precise orchestration of p62 oligomerization and spatial localization, thereby enhancing the sequestration of the targeted proteins within nascent autophagosomes. The utility of this inventive approach lies in its capacity to harness the cell's autophagic machinery, thereby offering a therapeutic modality with broad-spectrum applicability in the attenuation of diseases characterized by aberrant protein accumulation or defective protein clearance.

Throughout this disclosure, various patents, patent applications and publications are referenced. The disclosures of these patents, patent applications and publications in their entireties are incorporated into this disclosure by reference for all purposes in order to more fully describe the state of the art as known to those skilled therein as of the date of this disclosure. This disclosure will govern in the instance that there is any inconsistency between the patents, patent applications and publications cited and this disclosure.

### Definitions

Listed below are definitions of various terms used in the specification and claims to describe the present disclosure.

Unless defined otherwise, all technical and scientific terms used in this disclosure have the same meanings as commonly understood by one of ordinary skill in the art to which this disclosure belongs.

The term “about” when immediately preceding a numerical value means a range encompassing said numerical value plus or minus an acceptable amount of variation in the art (e.g., plus or minus 10% of that value). For example, “about 50” can mean 45 to 55, “about 25,000” can mean 22,500 to 27,500, etc., unless the context of the disclosure indicates otherwise, or is inconsistent with such an interpretation. For example in a list of numerical values such as “about 49, about 50, about 55, . . . ”, “about 50” means a range extending to less than half the interval(s) between the preceding and subsequent values, e.g., more than 49.5 to less than 50.5. Furthermore, the phrases “less than about” a value or “greater than about” a value should be understood in view of the definition of the term “about” provided herein. Similarly, the term “about” when preceding a series of numerical values or a range of values (e.g., “about 10, 20, 30” or “about 10-30”) refers, respectively to all values in the series, or the endpoints of the range.

When a range of values is listed, it is intended to encompass each value and sub-range within the range. For example, “C₁-C₆ alkyl” is intended to encompass C₁, C₂, C₃, C₄, C₅, C₆, C₁₋₆, C₁₋₅, C₁₋₄, C₁₋₃, C₁₋₂, C₂₋₆, C₂₋₅, C₂₋₄, C₂₋₃, C₃₋₆, C₃₋₅, C₃₋₄, C₄₋₆, C₄₋₅, and C₅₋₆ alkyl.

The terms below, as used herein, have the following meanings, unless indicated otherwise:

“Cyano” refers to the —CN radical.

“Hydroxy” or “hydroxyl” refers to the —OH radical.

“Oxo” refers to the ═O substituent.

“Halo,” “halogen” or “halide” refers to fluoro (—F), chloro (—Cl), bromo (—Br), and iodo (—I).

“Alkyl” or “alkyl group” refers to a fully saturated, straight or branched hydrocarbon chain radical having from one to twelve carbon atoms, and which is attached to the rest of the molecule by a single bond. Alkyls comprising any number of carbon atoms from 1 to 50 are included. An alkyl comprising up to 50 carbon atoms is a C₁-C₅₀ alkyl, an alkyl comprising up to 24 carbon atoms is a C₁-C₂₄ alkyl, an alkyl comprising up to 12 carbon atoms is a C₁-C₁₂ alkyl, an alkyl comprising up to 10 carbon atoms is a C₁-C₁₀ alkyl, an alkyl comprising up to 6 carbon atoms is a C₁-C₆ alkyl and an alkyl comprising up to 5 carbon atoms is a C₁-C₅ alkyl. A C₁-C₅ alkyl includes C₅ alkyls, C₄ alkyls, C₃ alkyls, C₂ alkyls and C₁ alkyl (i.e., methyl). A C₁-C₆ alkyl includes all moieties described above for C₁-C₅ alkyls but also includes C₆ alkyls. A C₁-C₁₀ alkyl includes all moieties described above for C₁-C₅ alkyls and C₁-C₆ alkyls, but also includes C₇, C₈, C₉ and C₁₀ alkyls. Similarly, a C₁-C₁₂ alkyl includes all the foregoing moieties, but also includes C₁₁ and C₁₂ alkyls. Non-limiting examples of C₁-C₁₂ alkyl include methyl, ethyl, n-propyl, i-propyl, sec-propyl, n-butyl, i-butyl, sec-butyl, t-butyl, n-pentyl, t-amyl, n-hexyl, n-heptyl, n-octyl, n-nonyl, n-decyl, n-undecyl, and n-dodecyl. Unless stated otherwise specifically in the specification, an alkyl group can be optionally substituted.

“Alkylene” or “alkylene chain” refers to a fully saturated, straight or branched divalent hydrocarbon chain radical, and having from 1 to 50 carbon atoms. Non-limiting examples of C₂-C₅₀ alkylene include ethylene, propylene, n-butylene, ethenylene, propenylene, n-butenylene, propynylene, n-butynylene, and the like. The alkylene chain is attached to the rest of the molecule through a single bond and to the radical group through a single bond. The points of attachment of the alkylene chain to the rest of the molecule and to the radical group can be through one carbon or any two carbons within the chain. Unless stated otherwise specifically in the specification, an alkylene chain can be optionally substituted. Non-limiting examples of substituted alkylene include —CH(CH₃)—, —CH₂—CH(CH₃)—, and the like.

“Alkenyl” or “alkenyl group” refers to a straight or branched hydrocarbon chain radical having from two to twelve carbon atoms, and having one or more carbon-carbon double bonds. Each alkenyl group is attached to the rest of the molecule by a single bond. Alkenyl group comprising any number of carbon atoms from 2 to 25 are included. An alkenyl group comprising up to 25 carbon atoms is a C₂-C₂₅ alkenyl, an alkenyl comprising up to 10 carbon atoms is a C₂-C₁₀ alkenyl, an alkenyl group comprising up to 6 carbon atoms is a C₂-C₆ alkenyl, and an alkenyl comprising up to 5 carbon atoms is a C₂-C₅ alkenyl. A C₂-C₅ alkenyl includes C₅ alkenyls, C₄ alkenyls, C₃ alkenyls, and C₂ alkenyls. A C₂-C₆ alkenyl includes all moieties described above for C₂-C₅ alkenyls but also includes C₆ alkenyls. A C₂-C₁₀ alkenyl includes all moieties described above for C₂-C₅ alkenyls and C₂-C₆ alkenyls, but also includes C₇, C₈, C₉ and C₁₀ alkenyls. Similarly, a C₂-C₁₂ alkenyl includes all the foregoing moieties, but also includes C₁₁ and C₁₂ alkenyls. Non-limiting examples of C₂-C₁₂ alkenyl include ethenyl (vinyl), 1-propenyl, 2-propenyl (allyl), iso-propenyl, 2-methyl-1-propenyl, 1-butenyl, 2-butenyl, 3-butenyl, 1-pentenyl, 2-pentenyl, 3-pentenyl, 4-pentenyl, 1-hexenyl, 2-hexenyl, 3-hexenyl, 4-hexenyl, 5-hexenyl, 1-heptenyl, 2-heptenyl, 3-heptenyl, 4-heptenyl, 5-heptenyl, 6-heptenyl, 1-octenyl, 2-octenyl, 3-octenyl, 4-octenyl, 5-octenyl, 6-octenyl, 7-octenyl, 1-nonenyl, 2-nonenyl, 3-nonenyl, 4-nonenyl, 5-nonenyl, 6-nonenyl, 7-nonenyl, 8-nonenyl, 1-decenyl, 2-decenyl, 3-decenyl, 4-decenyl, 5-decenyl, 6-decenyl, 7-decenyl, 8-decenyl, 9-decenyl, 1-undecenyl, 2-undecenyl, 3-undecenyl, 4-undecenyl, 5-undecenyl, 6-undecenyl, 7-undecenyl, 8-undecenyl, 9-undecenyl, 10-undecenyl, 1-dodecenyl, 2-dodecenyl, 3-dodecenyl, 4-dodecenyl, 5-dodecenyl, 6-dodecenyl, 7-dodecenyl, 8-dodecenyl, 9-dodecenyl, 10-dodecenyl, and 11-dodecenyl. Unless stated otherwise specifically in the specification, an alkenyl group can be optionally substituted.

“Alkenylene” or “alkenylene chain” refers to a straight or branched divalent hydrocarbon chain radical, having from 2 to 25 carbon atoms, and having one or more carbon-carbon double bonds. Non-limiting examples of C₂-C₂₅ alkenylene include ethene, propene, butene, and the like. The alkenylene chain is attached to the rest of the molecule through a single bond and to the radical group through a single bond. The points of attachment of the alkenylene chain to the rest of the molecule and to the radical group can be through one carbon or any two carbons within the chain. Unless stated otherwise specifically in the specification, an alkenylene chain can be optionally substituted.

“Alkynyl” or “alkynyl group” refers to a straight or branched hydrocarbon chain radical having from 2 to 25 carbon atoms, and having one or more carbon-carbon triple bonds. Each alkynyl group is attached to the rest of the molecule by a single bond. Alkynyl group comprising any number of carbon atoms from 2 to 25 are included. An alkynyl group comprising up to 25 carbon atoms is a C₂-C₂₅ alkynyl, an alkynyl comprising up to 10 carbon atoms is a C₂-C₁₀ alkynyl, an alkynyl group comprising up to 6 carbon atoms is a C₂-C₆ alkynyl and an alkynyl comprising up to 5 carbon atoms is a C₂-C₅ alkynyl. A C₂-C₅ alkynyl includes C₅ alkynyls, C₄ alkynyls, C₃ alkynyls, and C₂ alkynyls. A C₂-C₆ alkynyl includes all moieties described above for C₂-C₅ alkynyls but also includes C₆ alkynyls. A C₂-C₁₀ alkynyl includes all moieties described above for C₂-C₅ alkynyls and C₂-C₆ alkynyls, but also includes C₇, C₈, C₉ and C₁₀ alkynyls. Similarly, a C₂-C₁₂ alkynyl includes all the foregoing moieties, but also includes C₁₁ and C₁₂ alkynyls. Non-limiting examples of C₂-C₂₅ alkynyl include ethynyl, propynyl, butynyl, pentynyl and the like. Unless stated otherwise specifically in the specification, an alkynyl group can be optionally substituted.

“Alkynylene” or “alkynylene chain” refers to a straight or branched divalent hydrocarbon chain radical, having from 2 to 25 carbon atoms, and having one or more carbon-carbon triple bonds. Non-limiting examples of C₂-C₂₅ alkynylene include ethynylene, propargylene and the like. The alkynylene chain is attached to the rest of the molecule through a single bond and to the radical group through a single bond. The points of attachment of the alkynylene chain to the rest of the molecule and to the radical group can be through one carbon or any two carbons within the chain. Unless stated otherwise specifically in the specification, an alkynylene chain can be optionally substituted.

“Alkoxy” refers to a radical of the formula —ORₐ where Rₐ is an alkyl, alkenyl or alkynyl radical as defined above containing one to twelve carbon atoms. Unless stated otherwise specifically in the specification, an alkoxy group can be optionally substituted.

“Hydroxyalkyl” refers to an alkyl radical, as defined above, that is substituted by one or more hydroxy groups. As used herein, the term “hydroxyalkyl” encompasses alkyls having a primary (terminal) hydroxy group, such as —CH₂OH, —CH₂CH₂OH, —CH₂CH₂CH₂OH, —CH₂CH(CH₃)CH₂OH, and —CH₂CH₂CH₂CH₂OH, those having branched (non-terminal) hydroxy groups, such as —CH(OH)CH₃, —CH₂CH(CH₃)OH, and those having both primary and branched hydroxy groups, such as —CH₂CH(OH)CH₂CH₂OH.

“Alkylamino” refers to a radical of the formula —NHRₐ or —NRₐRₐ where each Rₐ is, independently, an alkyl, alkenyl or alkynyl radical as defined above containing one to twelve carbon atoms. Unless stated otherwise specifically in the specification, an alkylamino group can be optionally substituted.

“Aryl” refers to a hydrocarbon ring system radical comprising hydrogen, 6 to 18 carbon ring atoms and at least one aromatic ring. For purposes of this disclosure, the aryl radical can be a monocyclic, bicyclic, tricyclic or tetracyclic ring system, which can include fused, bridged, or spiro ring systems. Aryl radicals include, but are not limited to, aryl radicals derived from aceanthrylene, acenaphthylene, acephenanthrylene, anthracene, azulene, benzene, chrysene, fluoranthene, fluorene, as-indacene, s-indacene, indane, indene, naphthalene, phenalene, phenanthrene, pleiadene, pyrene, and triphenylene. Unless stated otherwise specifically in the specification, the term “aryl” is meant to include aryl radicals that are optionally substituted.

“Aralkyl” or “arylalkyl” refers to a radical of the formula —R_{b}—R_{c} where R_{b} is an alkylene group as defined above and R_{c} is one or more aryl radicals as defined above, for example, benzyl, diphenylmethyl and the like. Unless stated otherwise specifically in the specification, an arylalkyl group can be optionally substituted.

“Carbocycle,” “carbocyclyl,” or “carbocyclic ring” or refers to a ring structure, wherein the atoms which form the ring are each carbon. Carbocyclic rings can comprise from 3 to 20 carbon atoms in the ring. Carbocyclic rings include cycloalkyl, cycloalkenyl and cycloalkynyl as defined herein. Unless stated otherwise specifically in the specification, a carbocyclyl group can be optionally substituted.

“Cycloalkyl” refers to a stable non-aromatic monocyclic or polycyclic fully saturated hydrocarbon radical consisting solely of carbon and hydrogen atoms, which can include fused, bridged, or spiro ring systems, having from three to twenty carbon atoms, e.g., having from three to ten carbon atoms, and which is attached to the rest of the molecule by a single bond. Monocyclic cycloalkyl radicals include, for example, cyclopropyl, cyclobutyl, cyclopentyl, cyclohexyl, cycloheptyl, and cyclooctyl. Polycyclic cycloalkyl radicals include, for example, adamantyl, norbornyl, decalinyl, 7,7-dimethyl-bicyclo[2.2.1]heptanyl, and the like. Unless otherwise stated specifically in the specification, a cycloalkyl group can be optionally substituted.

“Cycloalkylene” refers to a divalent, non-aromatic, and fully saturated monocyclic or polycyclic hydrocarbon ring having 3 to 20 carbon atoms, or 3 to 8 carbon atoms. Non-limiting examples of C₃₋₈ cycloalkylene include

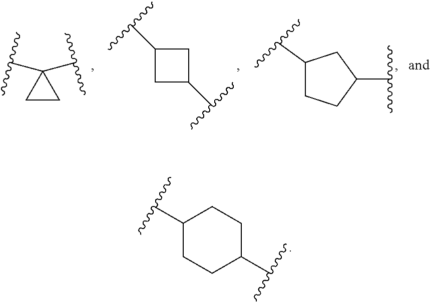 [SMILES: *C1(*)CC1 ; *C1CC(*)C1 ; *C1CCC(*)C1 ; *C1CCC(*)CC1]

“Cycloalkenyl” refers to a stable non-aromatic monocyclic or polycyclic hydrocarbon radical consisting solely of carbon and hydrogen atoms, having one or more carbon-carbon double bonds, which can include fused, bridged, or spiro ring systems, having from three to twenty carbon atoms, e.g., having from three to ten carbon atoms, and which is attached to the rest of the molecule by a single bond. Monocyclic cycloalkenyl radicals include, for example, cyclopentenyl, cyclohexenyl, cycloheptenyl, cycloctenyl, and the like. Polycyclic cycloalkenyl radicals include, for example, bicyclo[2.2.1]hept-2-enyl and the like. Unless otherwise stated specifically in the specification, a cycloalkenyl group can be optionally substituted.

“Cycloalkynyl” refers to a stable non-aromatic monocyclic or polycyclic hydrocarbon radical consisting solely of carbon and hydrogen atoms, having one or more carbon-carbon triple bonds, which can include fused, bridged, or spiro ring systems, having from three to twenty carbon atoms, e.g., having from three to ten carbon atoms, and which is attached to the rest of the molecule by a single bond. Monocyclic cycloalkynyl radicals include, for example, cycloheptynyl, cyclooctynyl, and the like. Unless otherwise stated specifically in the specification, a cycloalkynyl group can be optionally substituted.

“Cycloalkylalkyl” refers to a radical of the formula —R_{b}—R_{d} where R_{b} is an alkylene, alkenylene, or alkynylene group as defined above and R_{d} is a cycloalkyl, cycloalkenyl, cycloalkynyl radical as defined above. Unless stated otherwise specifically in the specification, a cycloalkylalkyl group can be optionally substituted.

“Haloalkyl” refers to an alkyl radical, as defined above, that is substituted by one or more halo radicals, as defined above, e.g., difluoromethyl, trifluoromethyl, trichloromethyl, 2,2,2-trifluoroethyl, 1,2-difluoroethyl, 3-bromo-2-fluoropropyl, 1,2-dibromoethyl, and the like. The haloalkyl group of the present disclosure can be e.g., a C₁₋₁₀ haloalkyl group, a C₁₋₆ haloalkyl group, or a C₁₋₃ haloalkyl group. Unless stated otherwise specifically in the specification, a haloalkyl group can be optionally substituted.

“Heterocyclyl” “heterocyclic ring” or “heterocycle” refers to a stable 3- to 20-membered non-aromatic, saturated or partially unsaturated ring radical which consists of two to twelve carbon ring atoms and from one to six heteroatoms as ring atoms selected from nitrogen, oxygen or sulfur, at least one non-aromatic, saturated or partially unsaturated ring containing at least one heteroatom as a ring atom. Unless stated otherwise specifically in the specification, the heterocyclyl radical can be a monocyclic, bicyclic, tricyclic or tetracyclic ring system, which can include fused, bridged, or spiro ring systems; and the nitrogen, carbon or sulfur atoms in the heterocyclyl radical can be optionally oxidized; the nitrogen atom can be optionally quaternized; and the heterocyclyl radical can be partially or fully saturated. Examples of such heterocyclyl radicals include, but are not limited to, dioxolanyl, thienyl[1,3]dithianyl, decahydroisoquinolyl, imidazolinyl, imidazolidinyl, isothiazolidinyl, isoxazolidinyl, morpholinyl, octahydroindolyl, octahydroisoindolyl, 2-oxopiperazinyl, 2-oxopiperidinyl, 2-oxopyrrolidinyl, oxazolidinyl, piperidinyl, piperazinyl, 4-piperidonyl, pyrrolidinyl, pyrazolidinyl, quinuclidinyl, thiazolidinyl, tetrahydrofuryl, trithianyl, tetrahydropyranyl, thiomorpholinyl, thiamorpholinyl, 1-oxo-thiomorpholinyl, and 1,1-dioxo-thiomorpholinyl. In embodiments where “L” is heterocyclyl, the heterocyclyl radical is a diradical. Unless stated otherwise specifically in the specification, a heterocyclyl group can be optionally substituted.

“Heteroaryl” refers to a 5- to 20-membered ring system radical comprising one to thirteen carbon ring atoms, one to six heteroatoms as ring atoms selected from nitrogen, oxygen and sulfur, and at least one aromatic ring containing at least one heteroatom as a ring atom. For purposes of this disclosure, the heteroaryl radical can be a monocyclic, bicyclic, tricyclic or tetracyclic ring system, which can include fused, bridged, or spiro ring systems; and the nitrogen, carbon or sulfur atoms in the heteroaryl radical can be optionally oxidized; the nitrogen atom can be optionally quaternized. Examples include, but are not limited to, azepinyl, acridinyl, benzimidazolyl, benzothiazolyl, benzindolyl, benzodioxolyl, benzofuranyl, benzooxazolyl, benzothiazolyl, benzothiadiazolyl, benzo[b][1,4]dioxepinyl, 1,4-benzodioxanyl, benzonaphthofuranyl, benzoxazolyl, benzodioxolyl, benzodioxinyl, benzopyranyl, benzopyranonyl, benzofuranyl, benzofuranonyl, benzothienyl (benzothiophene), benzotriazolyl, benzo[4,6]imidazo[1,2-a]pyridinyl, carbazolyl, cinnolinyl, dibenzofuranyl, dibenzothiophene, furanyl, furanonyl, isothiazolyl, imidazolyl, indazolyl, indolyl, indazolyl, isoindolyl, indolinyl, isoindolinyl, isoquinolyl, indolizinyl, isoxazolyl, naphthyridinyl, oxadiazolyl, 2-oxoazepinyl, oxazolyl, oxiranyl, 1-oxidopyridinyl, 1-oxidopyrimidinyl, 1-oxidopyrazinyl, 1-oxidopyridazinyl, 1-phenyl-1H-pyrrolyl, phenazinyl, phenothiazinyl, phenoxazinyl, phthalazinyl, pteridinyl, purinyl, pyrrolyl, pyrazolyl, pyridinyl, pyrazinyl, pyrimidinyl, pyridazinyl, quinazolinyl, quinoxalinyl, quinolinyl, quinuclidinyl, isoquinolinyl, tetrahydroquinolinyl, thiazolyl, thiadiazolyl, triazolyl, tetrazolyl, triazinyl, and thiophene (i.e. thienyl). Unless stated otherwise specifically in the specification, a heteroaryl group can be optionally substituted.

“Leaving group” refers to a functional group that can be substituted by another functional group during a chemical reaction. Exemplary leaving groups can be found in e.g., Organic Chemistry, Francis Carey, 2^{nd} edition, pages 328-331, McGraw-Hill Book Company, 1992, incorporated by reference herein. Non-limiting examples of leaving group include halogens (e.g., Cl, Br, I), methanesulfonyl (mesyl, Ms), p-toluenesulfonyl (tosyl, Ts), fluoromethanesulfonyl, difluoromethanesulfonyl, trifluoromethylsulfonyl (triflate, Tf), ethanesulfonyl, diazonium group,

“Protecting group” refers to a moiety that, when attached to a chemically reactive group in a molecule, masks or reduces chemical reactivity of the group. Protecting groups are well known in the art and include those described in detail in Protective Groups in Organic Synthesis, T. W. Greene, et al., 3^{rd} edition, John Wiley & Sons, 1999, incorporated by reference herein. Non-limiting examples of an amino protecting group (also referred to as a nitrogen protecting group) include those forming carbamates, such as tert-Butyloxycarbonyl (BOC) group, Carbobenzyloxy (Cbz) group, p-Methoxybenzyl carbonyl (Moz or MeOZ) group, Troc, 9-Fluorenylmethyloxycarbonyl (Fmoc) group, etc., those forming an amide, such as acetyl, trifluoroacetyl, benzoyl, etc., those forming a benzylic amine, such as benzyl, p-methoxybenzyl, 3,4-dimethoxybenzyl, etc., and others such as p-methoxyphenyl. Non-limiting examples of a hydroxy protecting group (also referred to as an oxygen protecting group) include those forming alkyl ethers or substituted alkyl ethers, such as methyl, allyl, benzyl, substituted benzyls such as 4-methoxybenzyl, methoxylmethyl (MOM), benzyloxymethyl (BOM), 2-methoxyethoxymethyl (MEM), etc., those forming silyl ethers, such as trymethylsilyl (TMS), triethylsilyl (TES), triisopropylsilyl (TIPS), t-butyldimethylsilyl (TBDMS), etc., those forming acetals or ketals, such as tetrahydropyranyl (THP), and those forming esters such as formate, acetate, chloroacetate, dichloroacetate, trichloroacetate, trifluoroacetate, methoxyacetate, etc.

The term “substituted” used herein means any of the above groups (i.e., alkyl, alkylene, alkenyl, alkenylene, alkynyl, alkynylene, alkoxy, alkylamino, thioalkyl, aryl, aralkyl, carbocyclyl, cycloalkyl, cycloalkenyl, cycloalkynyl, cycloalkylalkyl, haloalkyl, heterocyclyl, N-heterocyclyl, heterocyclylalkyl, heteroaryl, N-heteroaryl and/or heteroarylalkyl) wherein at least one hydrogen atom is replaced by a bond to a non-hydrogen atoms such as, but not limited to: a halogen atom such as F, Cl, Br, and I; an oxygen atom in groups such as hydroxyl groups, alkoxy groups, and ester groups; a sulfur atom in groups such as thiol groups, thioalkyl groups, sulfone groups, sulfonyl groups, and sulfoxide groups; a nitrogen atom in groups such as amines, amides, alkylamines, dialkylamines, arylamines, alkylarylamines, diarylamines, N-oxides, imides, and enamines; a silicon atom in groups such as trialkylsilyl groups, dialkylarylsilyl groups, alkyldiarylsilyl groups, and triarylsilyl groups; and other heteroatoms in various other groups.

“Substituted” also means any of the above groups in which one or more hydrogen atoms are replaced by a higher-order bond (e.g., a double- or triple-bond) to a heteroatom such as oxygen in oxo, carbonyl, carboxyl, and ester groups; and nitrogen in groups such as imines, oximes, hydrazones, and nitriles. For example, “substituted” includes any of the above groups in which one or more hydrogen atoms are replaced with —NR_{g}Rₕ, —NR_{g}C(═O)Rₕ, —NR_{g}C(═O)NR_{g}Rₕ, —NR_{g}C(═O)ORₕ, —NR_{g}SO₂Rₕ, —OC(═O)NR_{g}Rₕ, —OR_{g}, —SR_{g}, —SOR_{g}, —SO₂R_{g}, —OSO₂R_{g}, —SO₂OR_{g}, ═NSO₂R_{g}, and —SO₂NR_{g}Rₕ. “Substituted also means any of the above groups in which one or more hydrogen atoms are replaced with —C(═O)R_{g}, —C(═O)OR_{g}, —C(═O)NR_{g}Rₕ, —CH₂SO₂R_{g}, —CH₂SO₂NR_{g}Rₕ. In the foregoing, R_{g} and Rₕ are the same or different and independently hydrogen, alkyl, alkenyl, alkynyl, alkoxy, alkylamino, thioalkyl, aryl, aralkyl, cycloalkyl, cycloalkenyl, cycloalkynyl, cycloalkylalkyl, haloalkyl, haloalkenyl, haloalkynyl, heterocyclyl, N-heterocyclyl, heterocyclylalkyl, heteroaryl, N-heteroaryl and/or heteroarylalkyl. “Substituted” further includes any of the above groups in which one or more hydrogen atoms are replaced by a bond to an amino, cyano, hydroxyl, imino, nitro, oxo, thioxo, halo, alkyl, alkenyl, alkynyl, alkoxy, alkylamino, thioalkyl, aryl, aralkyl, cycloalkyl, cycloalkenyl, cycloalkynyl, cycloalkylalkyl, haloalkyl, haloalkenyl, haloalkynyl, heterocyclyl, N-heterocyclyl, heterocyclylalkyl, heteroaryl, N-heteroaryl and/or heteroarylalkyl group. In addition, each of the foregoing substituents can also be optionally substituted with one or more of the above substituents.

As used herein, the symbol

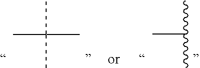

(hereinafter can be referred to as “a point of attachment bond”) denotes a bond that is a point of attachment between two chemical entities, one of which is depicted as being attached to the point of attachment bond and the other of which is not depicted as being attached to the point of attachment bond. For example,

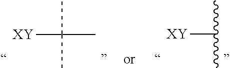

indicates that the chemical entity “XY” is bonded to another chemical entity via the point of attachment bond. Furthermore, the specific point of attachment to the non-depicted chemical entity can be specified by inference.

In this specification, unless stated otherwise, the term “pharmaceutically acceptable” is used to characterize a moiety (e.g., a salt, dosage form, or excipient) as being appropriate for use in accordance with sound medical judgment. In general, a pharmaceutically acceptable moiety has one or more benefits that outweigh any deleterious effect that the moiety may have. Deleterious effects may include, for example, excessive toxicity, irritation, allergic response, and other problems and complications.

The term “pharmaceutically acceptable salt” includes both acid and base addition salts. Pharmaceutically acceptable salts include those obtained by reacting the active compound functioning as a base, with an inorganic or organic acid to form a salt, for example, salts of hydrochloric acid, sulfuric acid, phosphoric acid, methanesulfonic acid, camphorsulfonic acid, oxalic acid, maleic acid, succinic acid, citric acid, formic acid, hydrobromic acid, benzoic acid, tartaric acid, fumaric acid, salicylic acid, mandelic acid, carbonic acid, etc. Those skilled in the art will further recognize that acid addition salts may be prepared by reaction of the compounds with the appropriate inorganic or organic acid via any of a number of known methods.

The compounds of the disclosure, or their pharmaceutically acceptable salts can contain one or more asymmetric centers and can thus give rise to enantiomers, diastereomers, and other stereoisomeric forms that can be defined, in terms of absolute stereochemistry, as (R)- or (S)- or, as (D)- or (L)- for amino acids. The present disclosure is meant to include all such possible isomers, as well as their racemic and optically pure forms whether or not they are specifically depicted herein. Optically active (+) and (−), (R)- and (S)-, or (D)- and (L)-isomers can be prepared using chiral synthons or chiral reagents, or resolved using conventional techniques, for example, chromatography and fractional crystallization. Conventional techniques for the preparation/isolation of individual enantiomers include chiral synthesis from a suitable optically pure precursor or resolution of the racemate (or the racemate of a salt or derivative) using, for example, chiral high pressure liquid chromatography (HPLC). When the compounds described herein contain olefinic double bonds or other centers of geometric asymmetry, and unless specified otherwise, it is intended that the compounds include both E and Z geometric isomers. Likewise, all tautomeric forms are also intended to be included.

The present disclosure is intended to encompass deuterated forms of the compounds described herein, which include isotopes of atoms occurring in the compounds. Isotopes include those atoms having the same atomic number but different mass numbers. By way of general example, and without limitation, isotopes of hydrogen include deuterium and tritium, and isotopes of carbon include ¹³C and ¹⁴C. Isotopically labeled compounds of the present disclosure can generally be prepared by conventional techniques known to those skilled in the art or by processes and methods analogous to those described herein, using an appropriate isotopically labeled reagent in place of the non-labeled reagent otherwise employed.

A “stereoisomer” refers to a compound made up of the same atoms bonded by the same bonds but having different three-dimensional structures, which are not interchangeable. The present disclosure contemplates various stereoisomers and mixtures thereof and includes “enantiomers”, which refers to two stereoisomers whose molecules are nonsuperimposable mirror images of one another.

A “derivative” refers to a chemically or biologically modified version of a chemical compound that is structurally similar to a parent compound and derivable from that parent compound. Derivatization (i.e., modification) may involve substitution of one or more moieties within the parent compound (e.g., a change in functional group). For example, when ligand A of the present disclosure is a derivative of a compound, ligand A can have a structure in which part of the structure of the compound is modified by binding to linker L². Exemplary modifications include replacement of a substituent (e.g., H, halogen, etc.) for subsequent formation of a bond via chemical process such as amidation, amination, acylation, alkylation, esterification, or dehydration.

The terms “subject,” “individual,” and “patient” are used interchangeably herein to refer to a vertebrate, such as a mammal. The mammal may be, for example, a mouse, a rat, a rabbit, a cat, a dog, a pig, a sheep, a horse, a non-human primate (e.g., cynomolgus monkey, chimpanzee), or a human.

The term “treating” as used herein with regard to a patient, refers to improving at least one symptom of the patient's disorder. Treating can be improving, or at least partially ameliorating a disorder or an associated symptom of a disorder.

An “effective amount” means the amount compound or pharmaceutical formulation, that when administered to a patient for treating a state, disorder or condition is sufficient to affect such treatment.

The term “therapeutically effective” applied to dose or amount refers to that quantity of a compound or pharmaceutical formulation that is sufficient to result in a desired clinical benefit after administration to a patient in need thereof. A “therapeutically effective amount”, in some embodiments, is a dose or amount of a compound or pharmaceutical formulation that is sufficient to result in prophylaxis after administration to a patient in need thereof.

It is further noted that the claims may be drafted to exclude any optional element. As such, this statement is intended to serve as antecedent basis for use of such exclusive terminology as “solely”, “only” and the like in connection with the recitation of claim elements, or the use of a “negative” limitation.

### Compounds

This disclosure presents compositions and methods for the manipulation of the intrinsic autophagic pathway for the selective degradation of pathogenic proteins. The disclosure comprises novel heterobifunctional compounds designed to engage the autophagy adaptor protein p62/SQSTM1, thereby triggering its activation and subsequent assembly of autophagosomes. These chimeric molecules are composed of a targeting ligand (also referred to as a “protein binding component” or “PBC”), which exhibits high-affinity binding to designated pathogenic proteins, conjoined via a designed, flexible linker to a p62 activating moiety (also referred to as a “warhead”). This bifunctional architecture enables the precise orchestration of p62 oligomerization and spatial localization, thereby enhancing the sequestration of the targeted proteins within nascent autophagosomes. The utility of this inventive approach lies in its capacity to harness the cell's autophagic machinery, thereby offering a therapeutic modality with broad-spectrum applicability in the attenuation of diseases characterized by aberrant protein accumulation or defective protein clearance.

In embodiments, the compounds of the present disclosure can be useful for targeted protein degradation, including for inducing targeted autophagy. In embodiments, the compounds of the present disclosure can be also useful for modulating activity of p62.

In one aspect, the disclosure provides compounds that target p62 and thereby modulating autophagy (e.g., compounds of formula (X-I), (I), (I-A), (I-B), (I-C), (I-D), (I-A-1), (I-A-2), (I-A-3), (I-A-4), (I-B-1), (I-B-2), (I-B-3), (I-B-4), (I-C-1), (I-C-2), (I-C-3), (I-C-4), (I-D-1), (I-D-2), (I-D-3), (I-D-4), (I-A-1-a), (I-A-2-a), (I-A-3-a), (I-B-1-a), (I-B-2-a), (I-B-3-a), (I-C-1-a), (I-D-1-a), (X-II), (II), (II-A), (II-B), (II-C), (II-D), (II-A-1), (II-A-2), (II-A-3), (II-A-4), (II-B3-1), (II-B3-2), (II-B3-3), (II-B3-4), (II-C-1), (II-C-2), (II-C-3), (II-C-4), (II-D-1), (II-D-2), (II-D-3), (II-D-4)). These compounds can be used on their own as therapeutics for modulating autophagy and treating diseases such as neurodegeneration and cancer. Alternatively or additionally, these compounds can be utilized in targeted protein degradation by functioning as adapter protein warheads (hereinafter “warheads”) that engage p62, and thereby bring p62 into proximity with a protein targeted for degradation. To leverage p62 for targeted protein degradation, in aspects, the disclosure provides for bifunctional compounds.

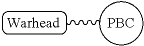

As illustrated above, these bifunctional compounds contain: (1) a first component (“warhead”) that targets and recruits an autophagy adaptor such as p62, and (2) a second component (“protein binding component” or “PBC”) that binds to a protein target to be degraded. In some embodiments, ligand A in the formulas described herein is a PBC. In embodiments, bifunctional compounds contain (3) a linker that covalently couples the warhead to the protein binding component. As such, the compounds disclosed herein can be applied for therapeutically degrading any specific targets, including, but not limited to, proteins, protein aggregates, protein complexes, lipids, lipid droplets, or pathogens (e.g., viruses) within the cell. Additional autophagy adaptor proteins include, but are not limited to, LC3, Optineurin, TAX1BP1, NBR1, NDP52, NUFIP1, WDFY3, RETREG1, Nix, and TOLLIP.

In some embodiments, ligand A and L², which will be detailed further below, function as the protein binding component (PBC) and linker, respectively, of the bifunctional compounds of formula (X-III), (III), (III-A), (III-B), (III-C), (III-D), (III-A-1), (III-A-2), (III-A-3), (III-A-4), (III-B3-1), (III-B-2), (III-B-3), (III-B-4), (III-C-1), (III-C-2), (III-C-3), (III-C-4), (III-D-1), (III-D-2), (III-D-3), or (III-D-4) described herein.

In embodiments, the present disclosure provides a compound of formula (X-I):

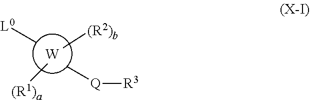

or a pharmaceutically acceptable salt, stereoisomer, or deuterated form thereof, wherein:
ring W is 6,5-fused heteroaryl or 6,5-fused heterocycle ring, wherein each ring W contains 1, 2, or 3 heteroatoms selected from N, O, or S, and at least 1 of the heteroatoms is N or O;
L⁰ is —C₁₋₆ alkylene-NR^{A}R^{B}, —C₁₋₆ alkylene-NR^{A}—C₁₋₆ alkylene-R^{B}, —C₁₋₆ alkylene-NR^{A}—C₃₋₈ cycloalkylene-R^{B}, —C₁₋₆ alkylene-NR^{A}C(O)NR^{A}R^{B}, —C₁₋₆ alkylene-NR^{A}C(O)—R^{B}, —C₁₋₆ alkylene-O—C₁₋₆ alkylene-NR^{A}R^{B}, —C₁₋₆ alkylene-O—C₁₋₆ alkylene-C(O)NR^{A}R^{B}, —C₁₋₆ alkylene-O—C₁₋₆ alkylene-NR^{A}C(O)—R^{B}, —C₁₋₆ alkylene-heterocyclylene-O—C₁₋₆ alkyl, —C₃₋₈ cycloalkylene-NR^{A}R^{B}, —C(O)NR^{A}R^{B}, —O—C₁₋₆ alkylene-C(O)NR^{A}R^{B}, —O—C₁₋₆ alkylene-NR^{A}R^{B}, —NR^{A}C(O)R^{B}—NR^{A}C(O)NR^{A}R^{B}, or —NR^{A}C(O)NR^{A}—C₁₋₆ alkylene-R^{B}, wherein the alkylene is optionally substituted with —OH or halogen;
each R¹ is independently C₁₋₆ alkyl, C₃-C₈ cycloalkyl, or halogen;
each R² is independently C₁₋₆ alkyl, C₃-C₈ cycloalkyl, or halogen, or two R² form an oxo;
Q is absent, C₁₋₆ alkylene, —C₁₋₆ alkylene-C(O)—, C₃₋₈ cycloalkylene, —C₃₋₈ cycloalkylene-C(O)—, —C(O)—, or —S(O)₂—;
R³ is carbocycle, heterocycle, aryl, heteroaryl, or —NR^{X}R^{Y}, wherein the carbocycle, heterocycle, aryl, and heteroaryl are each optionally substituted with 1, 2, or 3 R⁴;
each R⁴ is independently halogen, —OH, C₁₋₆ alkyl, C₁₋₆ alkoxy, —C(O)—NR^{X}R^{Y}, or —C(O)O—(C₁₋₆ alkyl);
each R^{A} is independently H, C₁₋₆ alkyl, C₁₋₆ haloalkyl, C₃₋₈ cycloalkyl, C₃₋₈ hydroxycycloalkyl, aryl, or heterocycle;
each R^{B} is independently C₁₋₆ alkyl, C₁₋₆ hydroxyalkyl, C₁₋₆ alkoxy, C₃₋₈ hydroxycycloalkyl, C₁₋₆ alkylene-NH₂, or C₁₋₆ alkylene-SH;
R^{X} is H, C₁₋₆ alkyl, C₁₋₆ haloalkyl, or C₁₋₆ hydroxyalkyl;
R^{Y} is C₁₋₆ alkyl, C₃₋₈ cycloalkyl, C₃₋₈ hydroxycycloalkyl, aryl, or heterocycle;
a is an integer of 0-3; and
b is an integer of 0-2.

In embodiments, the present disclosure provides a compound of formula (X-I):

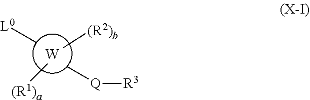

or a pharmaceutically acceptable salt, stereoisomer, or deuterated form thereof, wherein:
ring W is 6,5-fused heteroaryl or 6,5-fused heterocycle ring, wherein each ring W contains 1, 2, or 3 heteroatoms selected from N, O, or S, and at least 1 of the heteroatoms is N or O;
L⁰ is —C₁₋₆ alkylene-NR^{A}R^{B}, —C₁₋₆ alkylene-NR^{A}—C₁₋₆ alkylene-R^{B}, —C₁₋₆ alkylene-NR^{A}C(O)NR^{A}R^{B}, —C₁₋₆ alkylene-NR^{A}C(O)—R^{B}, —C₁₋₆ alkylene-O—C₁₋₆ alkylene-NR^{A}R^{B}, —C₃₋₈ cycloalkylene-NR^{A}R^{B}, —C(O)NR^{A}R^{B}, —O—C₁₋₆ alkylene-C(O)NR^{A}R^{B}, —O—C₁₋₆ alkylene-NR^{A}R^{B}, —NR^{A}C(O)R^{B}—NR^{A}C(O)NR^{A}R^{B}, or —NR^{A}C(O)NR^{A}—C₁₋₆ alkylene-R^{B}, wherein the alkylene is optionally substituted with —OH or halogen;
each R¹ is independently C₁₋₆ alkyl, C₃-C₈ cycloalkyl, or halogen;
each R² is independently C₁₋₆ alkyl, C₃-C₈ cycloalkyl, or halogen, or two R² form an oxo;
Q is absent, C₁₋₆ alkylene, —C₁₋₆ alkylene-C(O)—, C₃₋₈ cycloalkylene, —C₃₋₈ cycloalkylene-C(O)—, —C(O)—, or —S(O)₂—;
R³ is carbocycle, heterocycle, aryl, heteroaryl, or —NR^{X}R^{Y}, wherein the carbocycle, heterocycle, aryl, and heteroaryl are each optionally substituted with 1, 2, or 3 R⁴;
each R⁴ is independently halogen, —OH, C₁₋₆ alkyl, C₁₋₆ alkoxy, —C(O)—NR^{X}R^{Y}, or —C(O)O—(C₁₋₆ alkyl);
each R^{A} is independently H, C₁₋₆ alkyl, C₁₋₆ haloalkyl, C₃₋₈ cycloalkyl, C₃₋₈ hydroxycycloalkyl, aryl, or heterocycle;
each R^{B} is independently C₁₋₆ hydroxyalkyl, C₁₋₆ alkoxy, C₁₋₆ alkylene-NH₂, or C₁₋₆ alkylene-SH;
R^{X} is H, C₁₋₆ alkyl, C₁₋₆ haloalkyl, or C₁₋₆ hydroxyalkyl;
R^{Y} is C₁₋₆ alkyl, C₃₋₈ cycloalkyl, C₃₋₈ hydroxycycloalkyl, aryl, or heterocycle;
a is an integer of 0-3; and
b is an integer of 0-2.

In embodiments, the present disclosure provides a compound of formula (I):

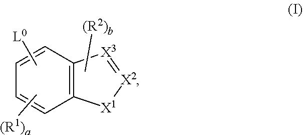

or a pharmaceutically acceptable salt, stereoisomer, or deuterated form thereof, wherein:
L⁰ is —C₁₋₆ alkylene-NR^{A}R^{B}, —C₁₋₆ alkylene-NR^{A}—C₁₋₆ alkylene-R^{B}, C₁₋₆ alkylene-NR^{A}—C₃₋₈ cycloalkylene-R^{B}, —C₁₋₆ alkylene-NR^{A}C(O)NR^{A}R^{B}, —C₁₋₆ alkylene-NR^{A}C(O)—R^{B}, —C₁₋₆ alkylene-O—C₁₋₆ alkylene-NR^{A}R^{B}, —C₁₋₆ alkylene-O—C₁₋₆ alkylene-C(O)NR^{A}R^{B}, —C₁₋₆ alkylene-O—C₁₋₆ alkylene-NR^{A}C(O)—R^{B}, —C₁₋₆ alkylene-heterocyclylene-O—C₁₋₆ alkyl, —C₃₋₈ cycloalkylene-NR^{A}R^{B}, —C(O)NR^{A}R^{B}, —O—C₁₋₆ alkylene-C(O)NR^{A}R^{B}, —O—C₁₋₆ alkylene-NR^{A}R^{B}, —NR^{A}C(O)R^{B}, —NR^{A}C(O)NR^{A}R^{B}, or —NR^{A}C(O)NR^{A}—C₁₋₆ alkylene-R^{B}, wherein the alkylene is optionally substituted with —OH or halogen;
each R¹ is independently C₁₋₆ alkyl, C₃-C₈ cycloalkyl, or halogen;
each R² is independently C₁₋₆ alkyl, C₃-C₈ cycloalkyl, or halogen;
X¹ is O or S;
one of X² or X³ is C-Q-R³ and the other is CH, C, or N as permitted by valency;
Q is absent, C₁₋₆ alkylene, —C₁₋₆ alkylene-C(O)—, C₃₋₈ cycloalkylene, —C₃₋₈ cycloalkylene-C(O)—, —C(O)—, or —S(O)₂—;
R³ is carbocycle, heterocycle, aryl, heteroaryl, or —NR^{X}R^{Y}, wherein the carbocycle, heterocycle, aryl, and heteroaryl are each optionally substituted with 1, 2, or 3 R⁴;
each R⁴ is independently halogen, —OH, C₁₋₆ alkyl, C₁₋₆ alkoxy, —C(O)—NR^{X}R^{Y}, or —C(O)O—(C₁₋₆ alkyl);
each R^{A} is independently H, C₁₋₆ alkyl, C₁₋₆ haloalkyl, C₃₋₈ cycloalkyl, C₃₋₈ hydroxycycloalkyl, aryl, or heterocycle;
each R^{B} is independently C₁₋₆ alkyl, C₁₋₆ hydroxyalkyl, C₁₋₆ alkoxy, C₃₋₈ hydroxycycloalkyl, C₁₋₆ alkylene-NH₂, or C₁₋₆ alkylene-SH;
R^{X} is H, C₁₋₆ alkyl, C₁₋₆ haloalkyl, or C₁₋₆ hydroxyalkyl;
R^{Y} is C₁₋₆ alkyl, C₃₋₈ cycloalkyl, C₃₋₈ hydroxycycloalkyl, aryl, or heterocycle;
a is an integer of 0-3; and
b is an integer of 0 or 1.

In embodiments, the present disclosure provides a compound of formula (I):

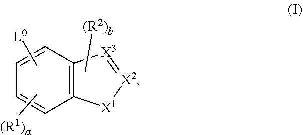

or a pharmaceutically acceptable salt, stereoisomer, or deuterated form thereof, wherein:
L⁰ is —C₁₋₆ alkylene-NR^{A}R^{B}, —C₁₋₆ alkylene-NR^{A}—C₁₋₆ alkylene-R^{B}, —C₁₋₆ alkylene-NR^{A}C(O)NR^{A}R^{B}, —C₁₋₆ alkylene-NR^{A}C(O)—R^{B}, —C₁₋₆ alkylene-O—C₁₋₆ alkylene-NR^{A}R^{B}, —C₃₋₈ cycloalkylene-NR^{A}R^{B}, —C(O)NR^{A}R^{B}, —O—C₁₋₆ alkylene-C(O)NR^{A}R^{B}, —O—C₁₋₆ alkylene-NR^{A}R^{B}, —NR^{A}C(O)R^{B}, —NR^{A}C(O)NR^{A}R^{B}, or —NR^{A}C(O)NR^{A}—C₁₋₆ alkylene-R^{B}, wherein the alkylene is optionally substituted with —OH or halogen;
each R¹ is independently C₁₋₆ alkyl, C₃-C₈ cycloalkyl, or halogen;
each R² is independently C₁₋₆ alkyl, C₃-C₈ cycloalkyl, or halogen;
X¹ is O or S;
one of X² or X³ is C-Q-R³ and the other is CH, C, or N as permitted by valency;
Q is absent, C₁₋₆ alkylene, —C₁₋₆ alkylene-C(O)—, C₃₋₈ cycloalkylene, —C₃₋₈ cycloalkylene-C(O)—, —C(O)—, or —S(O)₂—;
R³ is carbocycle, heterocycle, aryl, heteroaryl, or —NR^{X}R^{Y}, wherein the carbocycle, heterocycle, aryl, and heteroaryl are each optionally substituted with 1, 2, or 3 R⁴;
each R⁴ is independently halogen, —OH, C₁₋₆ alkyl, C₁₋₆ alkoxy, —C(O)—NR^{X}R^{Y}, or —C(O)O—(C₁₋₆ alkyl);
each R^{A} is independently H, C₁₋₆ alkyl, C₁₋₆ haloalkyl, C₃₋₈ cycloalkyl, C₃₋₈ hydroxycycloalkyl, aryl, or heterocycle;
each R^{B} is independently C₁₋₆ hydroxyalkyl, C₁₋₆ alkoxy, C₁₋₆ alkylene-NH₂, or C₁₋₆ alkylene-SH;
R^{X} is H, C₁₋₆ alkyl, C₁₋₆ haloalkyl, or C₁₋₆ hydroxyalkyl;
R^{Y} is C₁₋₆ alkyl, C₃₋₈ cycloalkyl, C₃₋₈ hydroxycycloalkyl, aryl, or heterocycle;
a is an integer of 0-3; and
b is an integer of 0 or 1.

In embodiments, the present disclosure provides a compound of formula (I-A):

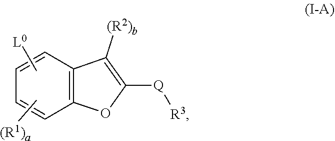

or a pharmaceutically acceptable salt, a stereoisomer, or a deuterated form thereof, wherein:
L⁰ is —C₁₋₆ alkylene-NR^{A}R^{B}, —C₁₋₆ alkylene-NR^{A}—C₁₋₆ alkylene-R^{B}, —C₁₋₆ alkylene-NR^{A}—C₃₋₈ cycloalkylene-R^{B}, —C₁₋₆ alkylene-NR^{A}C(O)NR^{A}R^{B}, —C₁₋₆ alkylene-NR^{A}C(O)—R^{B}, —C₁₋₆ alkylene-O—C₁₋₆ alkylene-NR^{A}R^{B}, —C₁₋₆ alkylene-O—C₁₋₆ alkylene-C(O)NR^{A}R^{B}, —C₁₋₆ alkylene-O—C₁₋₆ alkylene-NR^{A}C(O)—R^{B}, —C₁₋₆ alkylene-heterocyclylene-O—C₁₋₆ alkyl, —C₃₋₈ cycloalkylene-NR^{A}R^{B}, —C(O)NR^{A}R^{B}, —O—C₁₋₆ alkylene-C(O)NR^{A}R^{B}, —O—C₁₋₆ alkylene-NR^{A}R^{B}, —NR^{A}C(O)R^{B}, —NR^{A}C(O)NR^{A}R^{B}, or —NR^{A}C(O)NR^{A}—C₁₋₆ alkylene-R^{B}, wherein the alkylene is optionally substituted with —OH or halogen;
each R¹ is independently C₁₋₆ alkyl, C₃-C₈ cycloalkyl, or halogen;
each R² is independently C₁₋₆ alkyl, C₃-C₈ cycloalkyl, or halogen;
Q is absent, C₁₋₆ alkylene, —C₁₋₆ alkylene-C(O)—, C₃₋₈ cycloalkylene, —C₃₋₈ cycloalkylene-C(O)—, —C(O)—, or —S(O)₂—;
R³ is carbocycle, heterocycle, aryl, heteroaryl, or —NR^{X}R^{Y}, wherein the carbocycle, heterocycle, aryl, and heteroaryl are each optionally substituted with 1, 2, or 3 R⁴;
each R⁴ is independently halogen, —OH, C₁₋₆ alkyl, C₁₋₆ alkoxy, —C(O)—NR^{X}R^{Y}, or —C(O)O—(C₁₋₆ alkyl);
each R^{A} is independently H, C₁₋₆ alkyl, C₁₋₆ haloalkyl, C₃₋₈ cycloalkyl, C₃₋₈ hydroxycycloalkyl, aryl, or heterocycle;
each R^{B} is independently C₁₋₆ alkyl, C₁₋₆ hydroxyalkyl, C₁₋₆ alkoxy, C₃₋₈ hydroxycycloalkyl, C₁₋₆ alkylene-NH₂, or C₁₋₆ alkylene-SH;
R^{X} is H, C₁₋₆ alkyl, C₁₋₆ haloalkyl, or C₁₋₆ hydroxyalkyl;
R^{Y} is C₁₋₆ alkyl, C₃₋₈ cycloalkyl, C₃₋₈ hydroxycycloalkyl, aryl, or heterocycle;
a is an integer of 0-3; and
b is an integer of 0 or 1.

In embodiments, the present disclosure provides a compound of formula (I-A):

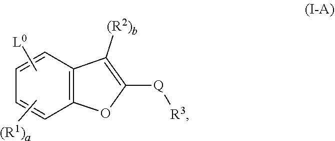

or a pharmaceutically acceptable salt, a stereoisomer, or a deuterated form thereof, wherein:
L⁰ is —C₁₋₆ alkylene-NR^{A}R^{B}, —C₁₋₆ alkylene-NR^{A}—C₁₋₆ alkylene-R^{B}, —C₁₋₆ alkylene-NR^{A}C(O)NR^{A}R^{B}, —C₁₋₆ alkylene-NR^{A}C(O)—R^{B}, —C₁₋₆ alkylene-O—C₁₋₆ alkylene-NR^{A}R^{B}, —C₃₋₈ cycloalkylene-NR^{A}R^{B}, —C(O)NR^{A}R^{B}, —O—C₁₋₆ alkylene-C(O)NR^{A}R^{B}, —O—C₁₋₆ alkylene-NR^{A}R^{B}, —NR^{A}C(O)R^{B}, —NR^{A}C(O)NR^{A}R^{B}, or —NR^{A}C(O)NR^{A}—C₁₋₆ alkylene-R^{B}, wherein the alkylene is optionally substituted with —OH or halogen;
each R¹ is independently C₁₋₆ alkyl, C₃-C₈ cycloalkyl, or halogen;
each R² is independently C₁₋₆ alkyl, C₃-C₈ cycloalkyl, or halogen;
Q is absent, C₁₋₆ alkylene, —C₁₋₆ alkylene-C(O)—, C₃₋₈ cycloalkylene, —C₃₋₈ cycloalkylene-C(O)—, —C(O)—, or —S(O)₂—;
R³ is carbocycle, heterocycle, aryl, heteroaryl, or —NR^{X}R^{Y}, wherein the carbocycle, heterocycle, aryl, and heteroaryl are each optionally substituted with 1, 2, or 3 R⁴;
each R⁴ is independently halogen, —OH, C₁₋₆ alkyl, C₁₋₆ alkoxy, —C(O)—NR^{X}R^{Y}, or —C(O)O—(C₁₋₆ alkyl);
each R^{A} is independently H, C₁₋₆ alkyl, C₁₋₆ haloalkyl, C₃₋₈ cycloalkyl, C₃₋₈ hydroxycycloalkyl, aryl, or heterocycle;
each R^{B} is independently C₁₋₆ hydroxyalkyl, C₁₋₆ alkoxy, C₁₋₆ alkylene-NH₂, or C₁₋₆ alkylene-SH;
R^{X} is H, C₁₋₆ alkyl, C₁₋₆ haloalkyl, or C₁₋₆ hydroxyalkyl;
R^{Y} is C₁₋₆ alkyl, C₃₋₈ cycloalkyl, C₃₋₈ hydroxycycloalkyl, aryl, or heterocycle;
a is an integer of 0-3; and
b is an integer of 0 or 1.

In embodiments, the present disclosure provides a compound of of formula (I-A-1), (I-A-2), (I-A-3), or (I-A-4):

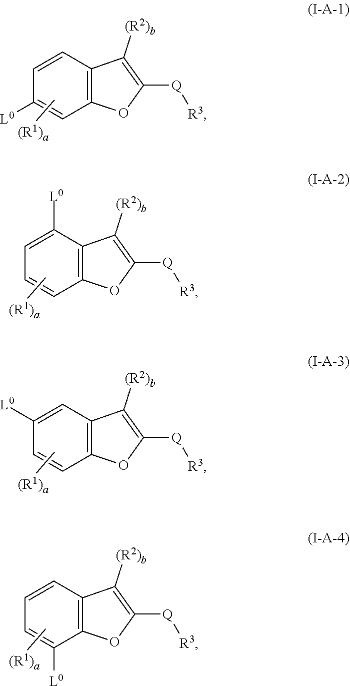

or a pharmaceutically acceptable salt, a stereoisomer, or a deuterated form thereof, wherein:
L⁰ is —C₁₋₆ alkylene-NR^{A}R^{B}, —C₁₋₆ alkylene-NR^{A}—C₁₋₆ alkylene-R^{B}, —C₁₋₆ alkylene-NR^{A}—C₃₋₈ cycloalkylene-R^{B}, —C₁₋₆ alkylene-NR^{A}C(O)NR^{A}R^{B}, —C₁₋₆ alkylene-NR^{A}C(O)—R^{B}, —C₁₋₆ alkylene-O—C₁₋₆ alkylene-NR^{A}R^{B}, —C₁₋₆ alkylene-O—C₁₋₆ alkylene-C(O)NR^{A}R^{B}, —C₁₋₆ alkylene-O—C₁₋₆ alkylene-NR^{A}C(O)—R^{B}, —C₁₋₆ alkylene-heterocyclylene-O—C₁₋₆ alkyl, —C₃₋₈ cycloalkylene-NR^{A}R^{B}, —C(O)NR^{A}R^{B}, —O—C₁₋₆ alkylene-C(O)NR^{A}R^{B}, —O—C₁₋₆ alkylene-NR^{A}R^{B}, —NR^{A}C(O)R^{B}, —NR^{A}C(O)NR^{A}R^{B}, or —NR^{A}C(O)NR^{A}—C₁₋₆ alkylene-R^{B}, wherein the alkylene is optionally substituted with —OH or halogen;
each R¹ is independently C₁₋₆ alkyl, C₃-C₈ cycloalkyl, or halogen;
each R² is independently C₁₋₆ alkyl, C₃-C₈ cycloalkyl, or halogen;
Q is absent, C₁₋₆ alkylene, —C₁₋₆ alkylene-C(O)—, C₃₋₈ cycloalkylene, —C₃₋₈ cycloalkylene-C(O)—, —C(O)—, or —S(O)₂—;
R³ is carbocycle, heterocycle, aryl, heteroaryl, or —NR^{X}R^{Y}, wherein the carbocycle, heterocycle, aryl, and heteroaryl are each optionally substituted with 1, 2, or 3 R⁴;
each R⁴ is independently halogen, —OH, C₁₋₆ alkyl, C₁₋₆ alkoxy, —C(O)—NR^{X}R^{Y}, or —C(O)O—(C₁₋₆ alkyl);
each R^{A} is independently H, C₁₋₆ alkyl, C₁₋₆ haloalkyl, C₃₋₈ cycloalkyl, C₃₋₈ hydroxycycloalkyl, aryl, or heterocycle;
each R^{B} is independently C₁₋₆ alkyl, C₁₋₆ hydroxyalkyl, C₁₋₆ alkoxy, C₃₋₈ hydroxycycloalkyl, C₁₋₆ alkylene-NH₂, or C₁₋₆ alkylene-SH;
R^{X} is H, C₁₋₆ alkyl, C₁₋₆ haloalkyl, or C₁₋₆ hydroxyalkyl;
R^{Y} is C₁₋₆ alkyl, C₃₋₈ cycloalkyl, C₃₋₈ hydroxycycloalkyl, aryl, or heterocycle;
a is an integer of 0-3; and
b is an integer of 0 or 1.

In embodiments, the present disclosure provides a compound of of formula (I-A-1), (I-A-2), (I-A-3), or (I-A-4):

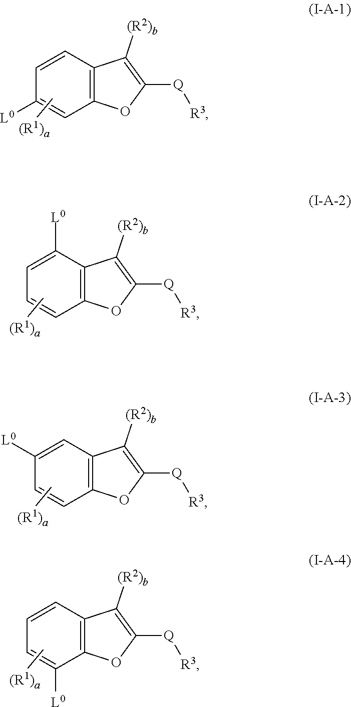

or a pharmaceutically acceptable salt, a stereoisomer, or a deuterated form thereof, wherein:
L⁰ is —C₁₋₆ alkylene-NR^{A}R^{B}, —C₁₋₆ alkylene-NR^{A}—C₁₋₆ alkylene-R^{B}, —C₁₋₆ alkylene-NR^{A}C(O)NR^{A}R^{B}, —C₁₋₆ alkylene-NR^{A}C(O)—R^{B}, —C₁₋₆ alkylene-O—C₁₋₆ alkylene-NR^{A}R^{B}, —C₃₋₈ cycloalkylene-NR^{A}R^{B}, —C(O)NR^{A}R^{B}, —O—C₁₋₆ alkylene-C(O)NR^{A}R^{B}, —O—C₁₋₆ alkylene-NR^{A}R^{B}, —NR^{A}C(O)R^{B}, —NR^{A}C(O)NR^{A}R^{B}, or —NR^{A}C(O)NR^{A}—C₁₋₆ alkylene-R^{B}, wherein the alkylene is optionally substituted with —OH or halogen;
each R¹ is independently C₁₋₆ alkyl, C₃-C₈ cycloalkyl, or halogen;
each R² is independently C₁₋₆ alkyl, C₃-C₈ cycloalkyl, or halogen;
Q is absent, C₁₋₆ alkylene, —C₁₋₆ alkylene-C(O)—, C₃₋₈ cycloalkylene, —C₃₋₈ cycloalkylene-C(O)—, —C(O)—, or —S(O)₂—;
R³ is carbocycle, heterocycle, aryl, heteroaryl, or —NR^{X}R^{Y}, wherein the carbocycle, heterocycle, aryl, and heteroaryl are each optionally substituted with 1, 2, or 3 R⁴;
each R⁴ is independently halogen, —OH, C₁₋₆ alkyl, C₁₋₆ alkoxy, —C(O)—NR^{X}R^{Y}, or —C(O)O—(C₁₋₆ alkyl);
each R^{A} is independently H, C₁₋₆ alkyl, C₁₋₆ haloalkyl, C₃₋₈ cycloalkyl, C₃₋₈ hydroxycycloalkyl, aryl, or heterocycle;
each R^{B} is independently C₁₋₆ hydroxyalkyl, C₁₋₆ alkoxy, C₁₋₆ alkylene-NH₂, or C₁₋₆ alkylene-SH;
R^{X} is H, C₁₋₆ alkyl, C₁₋₆ haloalkyl, or C₁₋₆ hydroxyalkyl;
R^{Y} is C₁₋₆ alkyl, C₃₋₈ cycloalkyl, C₃₋₈ hydroxycycloalkyl, aryl, or heterocycle;
a is an integer of 0-3; and
b is an integer of 0 or 1.

In embodiments, the present disclosure provides a compound of formula (I-B):

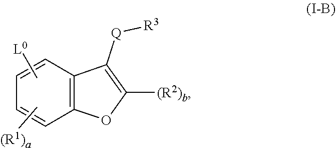

or a pharmaceutically acceptable salt, a stereoisomer, or a deuterated form thereof, wherein:
L⁰ is —C₁₋₆ alkylene-NR^{A}R^{B}, —C₁₋₆ alkylene-NR^{A}—C₁₋₆ alkylene-R^{B}, —C₁₋₆ alkylene-NR^{A}—C₃₋₈ cycloalkylene-R^{B}, —C₁₋₆ alkylene-NR^{A}C(O)NR^{A}R^{B}, —C₁₋₆ alkylene-NR^{A}C(O)—R^{B}, —C₁₋₆ alkylene-O—C₁₋₆ alkylene-NR^{A}R^{B}, —C₁₋₆ alkylene-O—C₁₋₆ alkylene-C(O)NR^{A}R^{B}, —C₁₋₆ alkylene-O—C₁₋₆ alkylene-NR^{A}C(O)—R^{B}, —C₁₋₆ alkylene-heterocyclylene-O—C₁₋₆ alkyl, —C₃₋₈ cycloalkylene-NR^{A}R^{B}, —C(O)NR^{A}R^{B}, —O—C₁₋₆ alkylene-C(O)NR^{A}R^{B}, —O—C₁₋₆ alkylene-NR^{A}R^{B}, —NR^{A}C(O)R^{B}, —NR^{A}C(O)NR^{A}R^{B}, or —NR^{A}C(O)NR^{A}—C₁₋₆ alkylene-R^{B}, wherein the alkylene is optionally substituted with —OH or halogen;
each R¹ is independently C₁₋₆ alkyl, C₃-C₈ cycloalkyl, or halogen;
each R² is independently C₁₋₆ alkyl, C₃-C₈ cycloalkyl, or halogen;
Q is absent, C₁₋₆ alkylene, —C₁₋₆ alkylene-C(O)—, C₃₋₈ cycloalkylene, —C₃₋₈ cycloalkylene-C(O)—, —C(O)—, or —S(O)₂—;
R³ is carbocycle, heterocycle, aryl, heteroaryl, or —NR^{X}R^{Y}, wherein the carbocycle, heterocycle, aryl, and heteroaryl are each optionally substituted with 1, 2, or 3 R⁴;
each R⁴ is independently halogen, —OH, C₁₋₆ alkyl, C₁₋₆ alkoxy, —C(O)—NR^{X}R^{Y}, or —C(O)O—(C₁₋₆ alkyl);
each R^{A} is independently H, C₁₋₆ alkyl, C₁₋₆ haloalkyl, C₃₋₈ cycloalkyl, C₃₋₈ hydroxycycloalkyl, aryl, or heterocycle;
each R^{B} is independently C₁₋₆ alkyl, C₁₋₆ hydroxyalkyl, C₁₋₆ alkoxy, C₃₋₈ hydroxycycloalkyl, C₁₋₆ alkylene-NH₂, or C₁₋₆ alkylene-SH;
R^{X} is H, C₁₋₆ alkyl, C₁₋₆ haloalkyl, or C₁₋₆ hydroxyalkyl;
R^{Y} is C₁₋₆ alkyl, C₃₋₈ cycloalkyl, C₃₋₈ hydroxycycloalkyl, aryl, or heterocycle;
a is an integer of 0-3; and
b is an integer of 0 or 1.

In embodiments, the present disclosure provides a compound of formula (I-B):

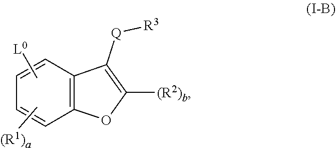

or a pharmaceutically acceptable salt, a stereoisomer, or a deuterated form thereof, wherein:
L⁰ is —C₁₋₆ alkylene-NR^{A}R^{B}, —C₁₋₆ alkylene-NR^{A}—C₁₋₆ alkylene-R^{B}, —C₁₋₆ alkylene-NR^{A}C(O)NR^{A}R^{B}, —C₁₋₆ alkylene-NR^{A}C(O)—R^{B}, —C₁₋₆ alkylene-O—C₁₋₆ alkylene-NR^{A}R^{B}, —C₃₋₈ cycloalkylene-NR^{A}R^{B}, —C(O)NR^{A}R^{B}, —O—C₁₋₆ alkylene-C(O)NR^{A}R^{B}, —O—C₁₋₆ alkylene-NR^{A}R^{B}, —NR^{A}C(O)R^{B}, —NR^{A}C(O)NR^{A}R^{B}, or —NR^{A}C(O)NR^{A}—C₁₋₆ alkylene-R^{B}, wherein the alkylene is optionally substituted with —OH or halogen;
each R¹ is independently C₁₋₆ alkyl, C₃-C₈ cycloalkyl, or halogen;
each R² is independently C₁₋₆ alkyl, C₃-C₈ cycloalkyl, or halogen;
Q is absent, C₁₋₆ alkylene, —C₁₋₆ alkylene-C(O)—, C₃₋₈ cycloalkylene, —C₃₋₈ cycloalkylene-C(O)—, —C(O)—, or —S(O)₂—;
R³ is carbocycle, heterocycle, aryl, heteroaryl, or —NR^{X}R^{Y}, wherein the carbocycle, heterocycle, aryl, and heteroaryl are each optionally substituted with 1, 2, or 3 R⁴;
each R⁴ is independently halogen, —OH, C₁₋₆ alkyl, C₁₋₆ alkoxy, —C(O)—NR^{X}R^{Y}, or —C(O)O—(C₁₋₆ alkyl);
each R^{A} is independently H, C₁₋₆ alkyl, C₁₋₆ haloalkyl, C₃₋₈ cycloalkyl, C₃₋₈ hydroxycycloalkyl, aryl, or heterocycle;
each R^{B} is independently C₁₋₆ hydroxyalkyl, C₁₋₆ alkoxy, C₁₋₆ alkylene-NH₂, or C₁₋₆ alkylene-SH;
R^{X} is H, C₁₋₆ alkyl, C₁₋₆ haloalkyl, or C₁₋₆ hydroxyalkyl;
R^{Y} is C₁₋₆ alkyl, C₃₋₈ cycloalkyl, C₃₋₈ hydroxycycloalkyl, aryl, or heterocycle;
a is an integer of 0-3; and
b is an integer of 0 or 1.

In embodiments, the present disclosure provides a compound of formula (I-B-1), (I-B-2), (I-B-3), or (I-B-4):

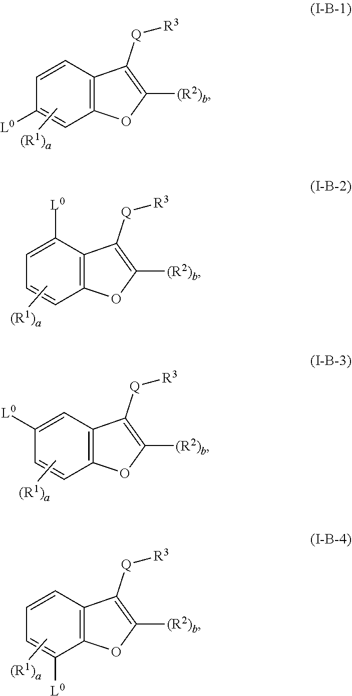

or a pharmaceutically acceptable salt, a stereoisomer, or a deuterated form thereof, wherein:
L⁰ is —C₁₋₆ alkylene-NR^{A}R^{B}, —C₁₋₆ alkylene-NR^{A}—C₁₋₆ alkylene-R^{B}, —C₁₋₆ alkylene-NR^{A}—C₃₋₈ cycloalkylene-R^{B}, —C₁₋₆ alkylene-NR^{A}C(O)NR^{A}R^{B}, —C₁₋₆ alkylene-NR^{A}C(O)—R^{B}, —C₁₋₆ alkylene-O—C₁₋₆ alkylene-NR^{A}R^{B}, —C₁₋₆ alkylene-O—C₁₋₆ alkylene-C(O)NR^{A}R^{B}, —C₁₋₆ alkylene-O—C₁₋₆ alkylene-NR^{A}C(O)—R^{B}, —C₁₋₆ alkylene-heterocyclylene-O—C₁₋₆ alkyl, —C₃₋₈ cycloalkylene-NR^{A}R^{B}, —C(O)NR^{A}R^{B}, —O—C₁₋₆ alkylene-C(O)NR^{A}R^{B}, —O—C₁₋₆ alkylene-NR^{A}R^{B}, —NR^{A}C(O)R^{B}, —NR^{A}C(O)NR^{A}R^{B}, or —NR^{A}C(O)NR^{A}—C₁₋₆ alkylene-R^{B}, wherein the alkylene is optionally substituted with —OH or halogen;
each R¹ is independently C₁₋₆ alkyl, C₃-C₈ cycloalkyl, or halogen;
each R² is independently C₁₋₆ alkyl, C₃-C₈ cycloalkyl, or halogen;
Q is absent, C₁₋₆ alkylene, —C₁₋₆ alkylene-C(O)—, C₃₋₈ cycloalkylene, —C₃₋₈ cycloalkylene-C(O)—, —C(O)—, or —S(O)₂—;
R³ is carbocycle, heterocycle, aryl, heteroaryl, or —NR^{X}R^{Y}, wherein the carbocycle, heterocycle, aryl, and heteroaryl are each optionally substituted with 1, 2, or 3 R⁴;
each R⁴ is independently halogen, —OH, C₁₋₆ alkyl, C₁₋₆ alkoxy, —C(O)—NR^{X}R^{Y}, or —C(O)O—(C₁₋₆ alkyl);
each R^{A} is independently H, C₁₋₆ alkyl, C₁₋₆ haloalkyl, C₃₋₈ cycloalkyl, C₃₋₈ hydroxycycloalkyl, aryl, or heterocycle;
each R^{B} is independently C₁₋₆ alkyl, C₁₋₆ hydroxyalkyl, C₁₋₆ alkoxy, C₃₋₈ hydroxycycloalkyl, C₁₋₆ alkylene-NH₂, or C₁₋₆ alkylene-SH;
R^{X} is H, C₁₋₆ alkyl, C₁₋₆ haloalkyl, or C₁₋₆ hydroxyalkyl;
R^{Y} is C₁₋₆ alkyl, C₃₋₈ cycloalkyl, C₃₋₈ hydroxycycloalkyl, aryl, or heterocycle;
a is an integer of 0-3; and
b is an integer of 0 or 1.

In embodiments, the present disclosure provides a compound of formula (I-B-1), (I-B-2), (I-B-3), or (I-B-4):

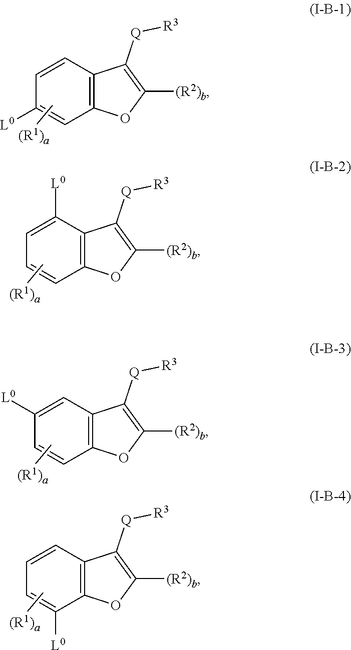

or a pharmaceutically acceptable salt, a stereoisomer, or a deuterated form thereof, wherein:
L⁰ is —C₁₋₆ alkylene-NR^{A}R^{B}, —C₁₋₆ alkylene-NR^{A}—C₁₋₆ alkylene-R^{B}, —C₁₋₆ alkylene-NR^{A}C(O)NR^{A}R^{B}, —C₁₋₆ alkylene-NR^{A}C(O)—R^{B}, —C₁₋₆ alkylene-O—C₁₋₆ alkylene-NR^{A}R^{B}, —C₃₋₈ cycloalkylene-NR^{A}R^{B}, —C(O)NR^{A}R^{B}, —O—C₁₋₆ alkylene-C(O)NR^{A}R^{B}, —O—C₁₋₆ alkylene-NR^{A}R^{B}, —NR^{A}C(O)R^{B}, —NR^{A}C(O)NR^{A}R^{B}, or —NR^{A}C(O)NR^{A}—C₁₋₆ alkylene-R^{B}, wherein the alkylene is optionally substituted with —OH or halogen;
each R¹ is independently C₁₋₆ alkyl, C₃-C₈ cycloalkyl, or halogen;
each R² is independently C₁₋₆ alkyl, C₃-C₈ cycloalkyl, or halogen;
Q is absent, C₁₋₆ alkylene, —C₁₋₆ alkylene-C(O)—, C₃₋₈ cycloalkylene, —C₃₋₈ cycloalkylene-C(O)—, —C(O)—, or —S(O)₂—;
R³ is carbocycle, heterocycle, aryl, heteroaryl, or —NR^{X}R^{Y}, wherein the carbocycle, heterocycle, aryl, and heteroaryl are each optionally substituted with 1, 2, or 3 R⁴;
each R⁴ is independently halogen, —OH, C₁₋₆ alkyl, C₁₋₆ alkoxy, —C(O)—NR^{X}R^{Y}, or —C(O)O—(C₁₋₆ alkyl);
each R^{A} is independently H, C₁₋₆ alkyl, C₁₋₆ haloalkyl, C₃₋₈ cycloalkyl, C₃₋₈ hydroxycycloalkyl, aryl, or heterocycle;
each R^{B} is independently C₁₋₆ hydroxyalkyl, C₁₋₆ alkoxy, C₁₋₆ alkylene-NH₂, or C₁₋₆ alkylene-SH;
R^{X} is H, C₁₋₆ alkyl, C₁₋₆ haloalkyl, or C₁₋₆ hydroxyalkyl;
R^{Y} is C₁₋₆ alkyl, C₃₋₈ cycloalkyl, C₃₋₈ hydroxycycloalkyl, aryl, or heterocycle;
a is an integer of 0-3; and
b is an integer of 0 or 1.

In embodiments, the present disclosure provides a compound of formula (I-C) or (I-D):

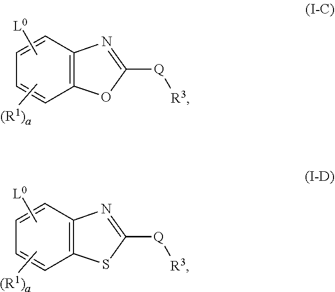

or a pharmaceutically acceptable salt, a stereoisomer, or a deuterated form thereof, wherein:
L⁰ is —C₁₋₆ alkylene-NR^{A}R^{B}, —C₁₋₆ alkylene-NR^{A}—C₁₋₆ alkylene-R^{B}, —C₁₋₆ alkylene-NR^{A}—C₃₋₈ cycloalkylene-R^{B}, —C₁₋₆ alkylene-NR^{A}C(O)NR^{A}R^{B}, —C₁₋₆ alkylene-NR^{A}C(O)—R^{B}, —C₁₋₆ alkylene-O—C₁₋₆ alkylene-NR^{A}R^{B}, —C₁₋₆ alkylene-O—C₁₋₆ alkylene-C(O)NR^{A}R^{B}, —C₁₋₆ alkylene-O—C₁₋₆ alkylene-NR^{A}C(O)—R^{B}, —C₁₋₆ alkylene-heterocyclylene-O—C₁₋₆ alkyl, —C₃₋₈ cycloalkylene-NR^{A}R^{B}, —C(O)NR^{A}R^{B}, —O—C₁₋₆ alkylene-C(O)NR^{A}R^{B}, —O—C₁₋₆ alkylene-NR^{A}R^{B}, —NR^{A}C(O)R^{B}, —NR^{A}C(O)NR^{A}R^{B}, or —NR^{A}C(O)NR^{A}—C₁₋₆ alkylene-R^{B}, wherein the alkylene is optionally substituted with —OH or halogen;
each R¹ is independently C₁₋₆ alkyl, C₃-C₈ cycloalkyl, or halogen;
Q is absent, C₁₋₆ alkylene, —C₁₋₆ alkylene-C(O)—, C₃₋₈ cycloalkylene, —C₃₋₈ cycloalkylene-C(O)—, —C(O)—, or —S(O)₂—;
R³ is carbocycle, heterocycle, aryl, heteroaryl, or —NR^{X}R^{Y}, wherein the carbocycle, heterocycle, aryl, and heteroaryl are each optionally substituted with 1, 2, or 3 R⁴;
each R⁴ is independently halogen, —OH, C₁₋₆ alkyl, C₁₋₆ alkoxy, —C(O)—NR^{X}R^{Y}, or —C(O)O—(C₁₋₆ alkyl);
each R^{A} is independently H, C₁₋₆ alkyl, C₁₋₆ haloalkyl, C₃₋₈ cycloalkyl, C₃₋₈ hydroxycycloalkyl, aryl, or heterocycle;
each R^{B} is independently C₁₋₆ alkyl, C₁₋₆ hydroxyalkyl, C₁₋₆ alkoxy, C₃₋₈ hydroxycycloalkyl, C₁₋₆ alkylene-NH₂, or C₁₋₆ alkylene-SH;
R^{X} is H, C₁₋₆ alkyl, C₁₋₆ haloalkyl, or C₁₋₆ hydroxyalkyl;
R^{Y} is C₁₋₆ alkyl, C₃₋₈ cycloalkyl, C₃₋₈ hydroxycycloalkyl, aryl, or heterocycle; and
a is an integer of 0-3.

In embodiments, the present disclosure provides a compound of formula (I-C) or (I-D):

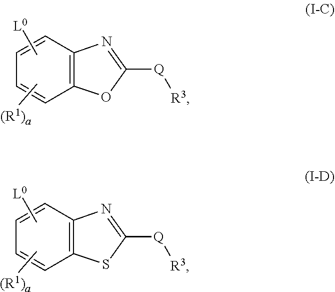

or a pharmaceutically acceptable salt, a stereoisomer, or a deuterated form thereof, wherein:
L⁰ is —C₁₋₆ alkylene-NR^{A}R^{B}, —C₁₋₆ alkylene-NR^{A}—C₁₋₆ alkylene-R^{B}, —C₁₋₆ alkylene-NR^{A}C(O)NR^{A}R^{B}, —C₁₋₆ alkylene-NR^{A}C(O)—R^{B}, —C₁₋₆ alkylene-O—C₁₋₆ alkylene-NR^{A}R^{B}, —C₃₋₈ cycloalkylene-NR^{A}R^{B}, —C(O)NR^{A}R^{B}, —O—C₁₋₆ alkylene-C(O)NR^{A}R^{B}, —O—C₁₋₆ alkylene-NR^{A}R^{B}, —NR^{A}C(O)R^{B}, —NR^{A}C(O)NR^{A}R^{B}, or —NR^{A}C(O)NR^{A}—C₁₋₆ alkylene-R^{B}, wherein the alkylene is optionally substituted with —OH or halogen;
each R¹ is independently C₁₋₆ alkyl, C₃-C₈ cycloalkyl, or halogen;
Q is absent, C₁₋₆ alkylene, —C₁₋₆ alkylene-C(O)—, C₃₋₈ cycloalkylene, —C₃₋₈ cycloalkylene-C(O)—, —C(O)—, or —S(O)₂—;
R³ is carbocycle, heterocycle, aryl, heteroaryl, or —NR^{X}R^{Y}, wherein the carbocycle, heterocycle, aryl, and heteroaryl are each optionally substituted with 1, 2, or 3 R⁴;
each R⁴ is independently halogen, —OH, C₁₋₆ alkyl, C₁₋₆ alkoxy, —C(O)—NR^{X}R^{Y}, or —C(O)O—(C₁₋₆ alkyl);
each R^{A} is independently H, C₁₋₆ alkyl, C₁₋₆ haloalkyl, C₃₋₈ cycloalkyl, C₃₋₈ hydroxycycloalkyl, aryl, or heterocycle;
each R^{B} is independently C₁₋₆ hydroxyalkyl, C₁₋₆ alkoxy, C₁₋₆ alkylene-NH₂, or C₁₋₆ alkylene-SH;
R^{X} is H, C₁₋₆ alkyl, C₁₋₆ haloalkyl, or C₁₋₆ hydroxyalkyl;
R^{Y} is C₁₋₆ alkyl, C₃₋₈ cycloalkyl, C₃₋₈ hydroxycycloalkyl, aryl, or heterocycle; and
a is an integer of 0-3.

In embodiments, the present disclosure provides a compound of formula (I-C-1), (I-C-2), (I-C-3), (I-C-4), (I-D-1), (I-D-2), (I-D-3), or (I-D-4):

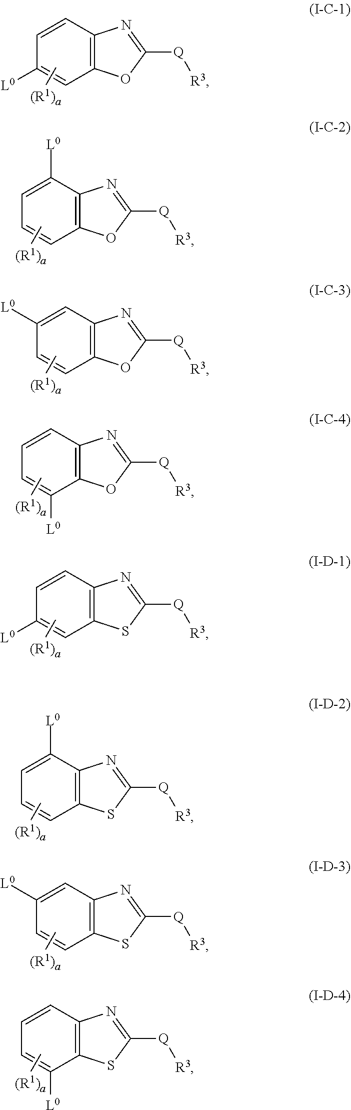

or a pharmaceutically acceptable salt, a stereoisomer, or a deuterated form thereof, wherein:
L⁰ is —C₁₋₆ alkylene-NR^{A}R^{B}, —C₁₋₆ alkylene-NR^{A}—C₁₋₆ alkylene-R^{B}, —C₁₋₆ alkylene-NR^{A}—C₃₋₈ cycloalkylene-R^{B}, —C₁₋₆ alkylene-NR^{A}C(O)NR^{A}R^{B}, —C₁₋₆ alkylene-NR^{A}C(O)—R^{B}, —C₁₋₆ alkylene-O—C₁₋₆ alkylene-NR^{A}R^{B}, —C₁₋₆ alkylene-O—C₁₋₆ alkylene-C(O)NR^{A}R^{B}, —C₁₋₆ alkylene-O—C₁₋₆ alkylene-NR^{A}C(O)—R^{B}, —C₁₋₆ alkylene-heterocyclylene-O—C₁₋₆ alkyl, —C₃₋₈ cycloalkylene-NR^{A}R^{B}, —C(O)NR^{A}R^{B}, —O—C₁₋₆ alkylene-C(O)NR^{A}R^{B}, —O—C₁₋₆ alkylene-NR^{A}R^{B}, —NR^{A}C(O)R^{B}, —NR^{A}C(O)NR^{A}R^{B}, or —NR^{A}C(O)NR^{A}—C₁₋₆ alkylene-R^{B}, wherein the alkylene is optionally substituted with —OH or halogen;
each R¹ is independently C₁₋₆ alkyl, C₃-C₈ cycloalkyl, or halogen;
Q is absent, C₁₋₆ alkylene, —C₁₋₆ alkylene-C(O)—, C₃₋₈ cycloalkylene, —C₃₋₈ cycloalkylene-C(O)—, —C(O)—, or —S(O)₂—;
R³ is carbocycle, heterocycle, aryl, heteroaryl, or —NR^{X}R^{Y}, wherein the carbocycle, heterocycle, aryl, and heteroaryl are each optionally substituted with 1, 2, or 3 R⁴;
each R⁴ is independently halogen, —OH, C₁₋₆ alkyl, C₁₋₆ alkoxy, —C(O)—NR^{X}R^{Y}, or —C(O)O—(C₁₋₆ alkyl);
each R^{A} is independently H, C₁₋₆ alkyl, C₁₋₆ haloalkyl, C₃₋₈ cycloalkyl, C₃₋₈ hydroxycycloalkyl, aryl, or heterocycle;
each R^{B} is independently C₁₋₆ alkyl, C₁₋₆ hydroxyalkyl, C₁₋₆ alkoxy, C₃₋₈ hydroxycycloalkyl, C₁₋₆ alkylene-NH₂, or C₁₋₆ alkylene-SH;
R^{X} is H, C₁₋₆ alkyl, C₁₋₆ haloalkyl, or C₁₋₆ hydroxyalkyl;
R^{Y} is C₁₋₆ alkyl, C₃₋₈ cycloalkyl, C₃₋₈ hydroxycycloalkyl, aryl, or heterocycle; and
a is an integer of 0-3.

In embodiments, the present disclosure provides a compound of formula (I-C-1), (I-C-2), (I-C-3), (I-C-4), (I-D-1), (I-D-2), (I-D-3), or (I-D-4):

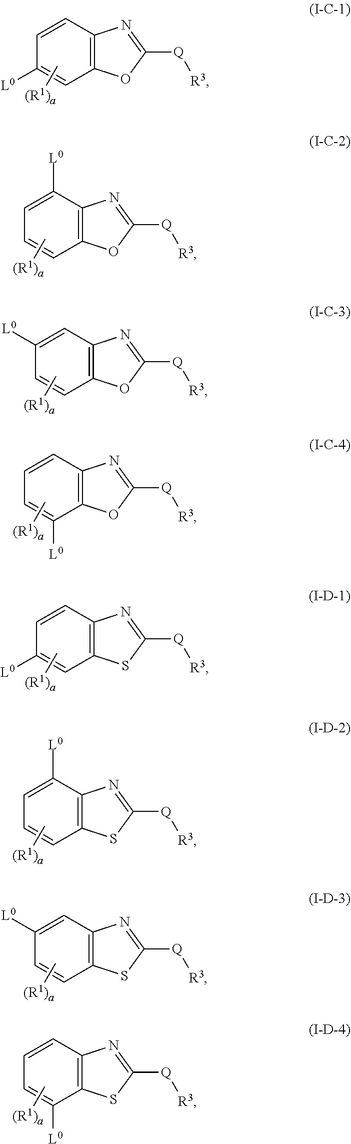

or a pharmaceutically acceptable salt, a stereoisomer, or a deuterated form thereof,
wherein:
L⁰ is —C₁₋₆ alkylene-NR^{A}R^{B}, —C₁₋₆ alkylene-NR^{A}—C₁₋₆ alkylene-R^{B}, —C₁₋₆ alkylene-NR^{A}C(O)NR^{A}R^{B}, —C₁₋₆ alkylene-NR^{A}C(O)—R^{B}, —C₁₋₆ alkylene-O—C₁₋₆ alkylene-NR^{A}R^{B}, —C₃₋₈ cycloalkylene-NR^{A}R^{B}, —C(O)NR^{A}R^{B}, —O—C₁₋₆ alkylene-C(O)NR^{A}R^{B}, —O—C₁₋₆ alkylene-NR^{A}R^{B}, —NR^{A}C(O)R^{B}, —NR^{A}C(O)NR^{A}R^{B}, or —NR^{A}C(O)NR^{A}—C₁₋₆ alkylene-R^{B}, wherein the alkylene is optionally substituted with —OH or halogen;
each R¹ is independently C₁₋₆ alkyl, C₃-C₈ cycloalkyl, or halogen;
Q is absent, C₁₋₆ alkylene, —C₁₋₆ alkylene-C(O)—, C₃₋₈ cycloalkylene, —C₃₋₈ cycloalkylene-C(O)—, —C(O)—, or —S(O)₂—;
R³ is carbocycle, heterocycle, aryl, heteroaryl, or —NR^{X}R^{Y}, wherein the carbocycle, heterocycle, aryl, and heteroaryl are each optionally substituted with 1, 2, or 3 R⁴;
each R⁴ is independently halogen, —OH, C₁₋₆ alkyl, C₁₋₆ alkoxy, —C(O)—NR^{X}R^{Y}, or —C(O)O—(C₁₋₆ alkyl);
each R^{A} is independently H, C₁₋₆ alkyl, C₁₋₆ haloalkyl, C₃₋₈ cycloalkyl, C₃₋₈ hydroxycycloalkyl, aryl, or heterocycle;
each R^{B} is independently C₁₋₆ hydroxyalkyl, C₁₋₆ alkoxy, C₁₋₆ alkylene-NH₂, or C₁₋₆ alkylene-SH;
R^{X} is H, C₁₋₆ alkyl, C₁₋₆ haloalkyl, or C₁₋₆ hydroxyalkyl;
R^{Y} is C₁₋₆ alkyl, C₃₋₈ cycloalkyl, C₃₋₈ hydroxycycloalkyl, aryl, or heterocycle; and
a is an integer of 0-3.

### L⁰

In embodiments of the compound of formula (X-I), (I), (I-A), (I-B), (I-C), (I-D), (I-A-1), (I-A-2), (I-A-3), (I-A-4), (I-B-1), (I-B-2), (I-B-3), (I-B-4), (I-C-1), (I-C-2), (I-C-3), (I-C-4), (I-D-1), (I-D-2), (I-D-3), or (I-D-4), or a pharmaceutically acceptable salt, stereoisomer, or deuterated form thereof, L⁰ is —C₁₋₆ alkylene-NR^{A}R^{B}, —C₁₋₆ alkylene-NR^{A}—C₁₋₆ alkylene-R^{B}, —C₁₋₆ alkylene-NR^{A}—C₃₋₈ cycloalkylene-R^{B}, —C₁₋₆ alkylene-NR^{A}C(O)NR^{A}R^{B}, —C₁₋₆ alkylene-NR^{A}C(O)—R^{B}, —C₁₋₆ alkylene-O—C₁₋₆ alkylene-NR^{A}R^{B}, —C₁₋₆ alkylene-O—C₁₋₆ alkylene-C(O)NR^{A}R^{B}, —C₁₋₆ alkylene-O—C₁₋₆ alkylene-NR^{A}C(O)—R^{B}, —C₁₋₆ alkylene-heterocyclylene-O—C₁₋₆ alkyl, —C₃₋₈ cycloalkylene-NR^{A}R^{B}, —C(O)NR^{A}R^{B}, —O—C₁₋₆ alkylene-C(O)NR^{A}R^{B}, —O—C₁₋₆ alkylene-NR^{A}R^{B}, —NR^{A}C(O)R^{B}, —NR^{A}C(O)NR^{A}R^{B}, or —NR^{A}C(O)NR^{A}—C₁₋₆ alkylene-R^{B}, wherein the alkylene is optionally substituted. In embodiments of the compound of formula (X-I), (I), (I-A), (I-B), (I-C), (I-D), (I-A-1), (I-A-2), (I-A-3), (I-A-4), (I-B-1), (I-B-2), (I-B-3), (I-B-4), (I-C-1), (I-C-2), (I-C-3), (I-C-4), (I-D-1), (I-D-2), (I-D-3), or (I-D-4), or a pharmaceutically acceptable salt, stereoisomer, or deuterated form thereof, L⁰ is —C₁₋₆ alkylene-NR^{A}R^{B}, —C₁₋₆ alkylene-NR^{A}—C₁₋₆ alkylene-R^{B}, —C₁₋₆ alkylene-NR^{A}C(O)NR^{A}R^{B}, —C₁₋₆ alkylene-NR^{A}C(O)—R^{B}, —C₁₋₆ alkylene-O—C₁₋₆ alkylene-NR^{A}R^{B}, —C₃₋₈ cycloalkylene-NR^{A}R^{B}, —C(O)NR^{A}R^{B}, —O—C₁₋₆ alkylene-C(O)NR^{A}R^{B}, —O—C₁₋₆ alkylene-NR^{A}R^{B}, —NR^{A}C(O)R^{B}, —NR^{A}C(O)NR^{A}R^{B}, or —NR^{A}C(O)NR^{A}—C₁₋₆ alkylene-R^{B}, wherein the alkylene is optionally substituted. In embodiments, the alkylene is optionally substituted with —OH, halogen, C₁₋₆ hydroxyalkyl, C₁₋₆ haloalkyl, C₃₋₈ cycloalkyl, C₁₋₆ alkoxy, —NH₂, NH(C₁-C₆ alkyl), N(C₁-C₆ alkyl)₂, S(C₁-C₆ alkyl), —SO(C₁₋₆ alkyl), —SO₂(C₁₋₆ alkyl), —SO₂N(C₁₋₆ alkyl), —SO₂NH₂, —SO₂NH(C₁-C₆ alkyl), —SO₂N(C₁-C₆ alkyl)₂, carbocycle, heterocycle, aryl, or heteroaryl. In embodiments, the alkylene is optionally substituted with —OH or halogen. In embodiments, each R^{A} is independently H, C₁₋₆ alkyl, C₁₋₆ haloalkyl, C₃₋₈ cycloalkyl, C₃₋₈ hydroxycycloalkyl, aryl, or heterocycle. In embodiments, each R^{B} is independently C₁₋₆ alkyl, C₁₋₆ hydroxyalkyl, C₁₋₆ alkoxy, C₃₋₈ hydroxycycloalkyl, C₁₋₆ alkylene-NH₂, or C₁₋₆ alkylene-SH. In embodiments, each R^{B} is independently C₁₋₆ hydroxyalkyl, C₁₋₆ alkoxy, C₁₋₆ alkylene-NH₂, or C₁₋₆ alkylene-SH.

In embodiments of the compound of formula (X-I), (I), (I-A), (I-B), (I-C), (I-D), (I-A-1), (I-A-2), (I-A-3), (I-A-4), (I-B-1), (I-B-2), (I-B-3), (I-B-4), (I-C-1), (I-C-2), (I-C-3), (I-C-4), (I-D-1), (I-D-2), (I-D-3), or (I-D-4), or a pharmaceutically acceptable salt, stereoisomer, or deuterated form thereof, L⁰ is —(C₁₋₆ alkylene)-N(H)(C₁₋₆ hydroxyalkyl), —(C₁₋₆ alkylene)-N(C₁₋₆ alkyl)(C₁₋₆ hydroxyalkyl), —(C₁₋₆ alkylene)-N(H)(C₁₋₆ alkoxy), —(C₁₋₆ alkylene)-N(H)(C₁₋₆ alkylene)(C₁₋₆ alkoxy), —C(O)N(H)(C₁₋₆ hydroxyalkyl), —(C₁₋₆ alkylene)-N(H)(C₃₋₈ cycloalkylene)(C₁₋₆ alkoxy), —(C₁₋₆ alkylene)-N(C₁₋₆ alkyl)(C₁₋₆ alkoxy), —(C₁₋₆ alkylene)-N(H)C(O)(C₁₋₆ alkoxy), —(C₁₋₆ alkylene)-N(C₁₋₆ alkyl)C(O)(C₁₋₆ alkoxy), —(C₁₋₆ alkylene)-N(H)C(O)N(H)(C₁₋₆ alkoxy), —(C₁₋₆ alkylene)-N(C₁₋₆ alkyl)C(O)N(C₁₋₆ alkyl)(C₁₋₆ alkoxy), —(C₃₋₈ cycloalkylene)N(H)(C₁₋₆ hydroxyalkyl), —(C₃₋₈ cycloalkylene)N(C₁₋₆ alkyl)(C₁₋₆ hydroxyalkyl), —O—(C₁₋₆ alkylene)-N(H)(C₁₋₆ hydroxyalkyl), —O—(C₁₋₆ alkylene)-N(C₁₋₆ alkyl)(C₁₋₆ hydroxyalkyl), —N(H)(C₁₋₆ hydroxyalkyl), —N(H)C(O)N(H)(C₁₋₆ hydroxyalkyl), —N(H)C(O)N(H)(C₁₋₆ alkylene)-(C₁₋₆ alkoxy), —(C₁₋₆ alkylene)-O—(C₁₋₆ alkylene)C(O)N(H)(C₁₋₆ alkyl), —(C₁₋₆ alkylene)-O—(C₁₋₆ alkylene)N(H)C(O)(C₁₋₆ alkyl), —O—(C₁₋₆ alkylene)C(O)N(H)(C₁₋₆ alkyl), —N(C₁₋₆ alkyl)(C₁₋₆ hydroxyalkyl), —N(H)C(O)(C₁₋₆ hydroxyalkyl), —N(C₁₋₆ alkyl)C(O)(C₁₋₆ hydroxyalkyl), —(C₁₋₆ alkylene)-heterocyclylene-(C₁₋₆ alkoxy), —(C₁₋₆ alkylene)-O—(C₁₋₆ alkylene)-N(H)(C₁₋₆ alkyl), or —(C₁₋₆ alkylene)-O—(C₁₋₆ alkylene)-N(C₁₋₆ alkyl)(C₁₋₆ alkyl), wherein the alkylene or alkyl is optionally substituted. In embodiments, the alkylene or alkyl is optionally substituted with —OH, halogen, C₁₋₆ alkyl, C₁₋₆ hydroxyalkyl, C₁₋₆ haloalkyl, C₃₋₈ cycloalkyl, C₁₋₆ alkoxy, —NH₂, NR^{A}R^{B}, —S(C₁-C₆ alkyl), —SO(C₁₋₆ alkyl), —SO₂(C₁₋₆ alkyl), —SO₂N(C₁₋₆ alkyl), —SO₂NR^{A}R^{B}, carbocycle, heterocycle, aryl, or heteroaryl. In embodiments of the compound of formula (X-I), (I), (I-A), (I-B), (I-C), (I-D), (I-A-1), (I-A-2), (I-A-3), (I-A-4), (I-B-1), (I-B-2), (I-B-3), (I-B-4), (I-C-1), (I-C-2), (I-C-3), (I-C-4), (I-D-1), (I-D-2), (I-D-3), or (I-D-4), or a pharmaceutically acceptable salt, stereoisomer, or deuterated form thereof, L⁰ is —(C₁₋₆ alkylene)-N(H)(C₁₋₆ hydroxyalkyl), —(C₁₋₆ alkylene)-N(C₁₋₆ alkyl)(C₁₋₆ hydroxyalkyl), —(C₁₋₆ alkylene)-N(H)(C₁₋₆ alkoxy), —(C₁₋₆ alkylene)-N(C₁₋₆ alkyl)(C₁₋₆ alkoxy), —(C₁₋₆ alkylene)-N(H)C(O)(C₁₋₆ alkoxy), —(C₁₋₆ alkylene)-N(C₁₋₆ alkyl)C(O)(C₁₋₆ alkoxy), —(C₁₋₆ alkylene)-N(H)C(O)N(H)(C₁₋₆ alkoxy), —(C₁₋₆ alkylene)-N(C₁₋₆ alkyl)C(O)N(C₁₋₆ alkyl)(C₁₋₆ alkoxy), —(C₃₋₈ cycloalkylene)N(H)(C₁₋₆ hydroxyalkyl), —(C₃₋₈ cycloalkylene)N(C₁₋₆ alkyl)(C₁₋₆ hydroxyalkyl), —O—(C₁₋₆ alkylene)-N(H)(C₁₋₆ hydroxyalkyl), —O—(C₁₋₆ alkylene)-N(C₁₋₆ alkyl)(C₁₋₆ hydroxyalkyl), —N(H)(C₁₋₆ hydroxyalkyl), —N(C₁₋₆ alkyl)(C₁₋₆ hydroxyalkyl), —N(H)C(O)(C₁₋₆ hydroxyalkyl), or —N(C₁₋₆ alkyl)C(O)(C₁₋₆ hydroxyalkyl), wherein the alkylene or alkyl is optionally substituted. In embodiments, the alkylene or alkyl is optionally substituted with —OH, halogen, C₁₋₆ alkyl, C₁₋₆ hydroxyalkyl, C₁₋₆ haloalkyl, C₃₋₈ cycloalkyl, C₁₋₆ alkoxy, —NH₂, NR^{A}R^{B}, —S(C₁-C₆ alkyl), —SO(C₁₋₆ alkyl), —SO₂(C₁₋₆ alkyl), —SO₂N(C₁₋₆ alkyl), —SO₂NR^{A}R^{B}, carbocycle, heterocycle, aryl, or heteroaryl. In some embodiments, the alkylene or alkyl is optionally substituted with —OH or halogen.

In embodiments of the compound of formula (I), (I-A), (I-B), (I-C), (I-D), (I-A-1), (I-A-2), (I-A-3), (I-A-4), (I-B-1), (I-B-2), (I-B-3), (I-B-4), (I-C-1), (I-C-2), (I-C-3), (I-C-4), (I-D-1), (I-D-2), (I-D-3), or (I-D-4), or a pharmaceutically acceptable salt, stereoisomer, or deuterated form thereof, L⁰ is —C₁₋₆ alkylene-NR^{A}R^{B}. In some embodiments, L⁰ is —C₁₋₃ alkylene-NR^{A}R^{B}. In some embodiments, each R^{A} is independently H, C₁₋₆ alkyl, C₁₋₆ haloalkyl, C₃₋₈ cycloalkyl, C₃₋₈ hydroxycycloalkyl, aryl, or heterocycle. In some embodiments, each R^{B} is independently C₁₋₆ alkyl, C₁₋₆ hydroxyalkyl, C₃₋₈ hydroxycycloalkyl, C₁₋₆ alkoxy, C₁₋₆ alkylene-NH₂, or C₁₋₆ alkylene-SH. In some embodiments, each R^{B} is independently C₁₋₆ hydroxyalkyl, C₁₋₆ alkoxy, C₁₋₆ alkylene-NH₂, or C₁₋₆ alkylene-SH. In some embodiments, R^{A} is H, and R^{B} is C₁₋₆ hydroxyalkyl or C₃₋₆ hydroxycycloalkyl. In some embodiments, R^{B} is —CH₂CH₂OH, —CH₂CH(CH₃)OH,

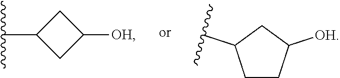 [SMILES: *C1CC(O)C1 ; *C1CCC(O)C1]

In some embodiments, L⁰ is —CH₂NHCH₂CH₂OH, —CH₂NHCH₂CH(CH₃)OH, —CH(CH₃)NHCH₂CH₂OH,

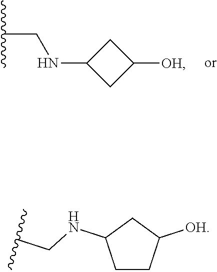 [SMILES: *CNC1CC(O)C1 ; *CNC1CCC(O)C1]

In some embodiments, L⁰ is —CH₂NHCH₂CH₂OH, —CH₂NHCH₂CH(CH₃)OH, or

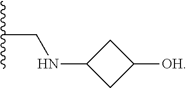 [SMILES: *CNC1CC(O)C1]

In some embodiments, L⁰ is —CH₂NHCH₂CH₂OH.

In embodiments of the compound of formula (I), (I-A), (I-B), (I-C), (I-D), (I-A-1), (I-A-2), (I-A-3), (I-A-4), (I-B-1), (I-B-2), (I-B-3), (I-B-4), (I-C-1), (I-C-2), (I-C-3), (I-C-4), (I-D-1), (I-D-2), (I-D-3), or (I-D-4), or a pharmaceutically acceptable salt, stereoisomer, or deuterated form thereof, L⁰ is C₁₋₆ alkylene-NR^{A}—C₁₋₆ alkylene-R^{B}. In some embodiments, each R^{B} is independently C₁₋₆ alkyl, C₁₋₆ hydroxyalkyl, C₃₋₈ hydroxycycloalkyl, C₁₋₆ alkoxy, C₁₋₆ alkylene-NH₂, or C₁₋₆ alkylene-SH. In some embodiments, R^{B} is C₁₋₆ alkoxy and L⁰ is —(C₁₋₆ alkylene)-NR^{A}—(C₁₋₆ alkylene)-(C₁₋₆ alkoxy). In some embodiments, R^{A} is H, C₁₋₆ alkyl, C₁₋₆ haloalkyl, C₃₋₈ cycloalkyl, C₃₋₈ hydroxycycloalkyl, aryl, or heterocycle. In some embodiments, R^{A} is H. In some embodiments, L⁰ is —(C₁₋₃ alkylene)-NH—(C₁₋₃ alkylene)-(C₁₋₄ alkoxy). In some embodiments, L⁰ is —CH₂NHCH₂CH₂OCH(CH₃)₂ or —CH₂NHCH₂CH₂OCH₃.

In embodiments of the compound of formula (I), (I-A), (I-B), (I-C), (I-D), (I-A-1), (I-A-2), (I-A-3), (I-A-4), (I-B-1), (I-B-2), (I-B-3), (I-B-4), (I-C-1), (I-C-2), (I-C-3), (I-C-4), (I-D-1), (I-D-2), (I-D-3), or (I-D-4), or a pharmaceutically acceptable salt, stereoisomer, or deuterated form thereof, L⁰ is —(C₁₋₆ alkylene)-NR^{A}—(C₁₋₆ alkylene)-(C₁₋₆ alkoxy). In some embodiments, R^{A} is H, C₁₋₆ alkyl, C₁₋₆ haloalkyl, C₃₋₈ cycloalkyl, C₃₋₈ hydroxycycloalkyl, aryl, or heterocycle. In some embodiments, R^{A} is H. In some embodiments, L⁰ is —(C₁₋₃ alkylene)-NH—(C₁₋₃ alkylene)-(C₂₋₄ alkoxy). In some embodiments, L⁰ is —CH₂NHCH₂CH₂OCH(CH₃)₂.

In embodiments of the compound of formula (I), (I-A), (I-B), (I-C), (I-D), (I-A-1), (I-A-2), (I-A-3), (I-A-4), (I-B-1), (I-B-2), (I-B-3), (I-B-4), (I-C-1), (I-C-2), (I-C-3), (I-C-4), (I-D-1), (I-D-2), (I-D-3), or (I-D-4), or a pharmaceutically acceptable salt, stereoisomer, or deuterated form thereof, L⁰ is —(C₁₋₆ alkylene)-O—(C₁₋₆ alkylene)-NR^{A}R^{B}. In some embodiments, R^{A} is H, C₁₋₆ alkyl, C₁₋₆ haloalkyl, C₃₋₈ cycloalkyl, C₃₋₈ hydroxycycloalkyl, aryl, or heterocycle. In some embodiments, R^{B} is C₁₋₆ alkyl, C₁₋₆ hydroxyalkyl, C₃₋₈ hydroxycycloalkyl, C₁₋₆ alkoxy, C₁₋₆ alkylene-NH₂, or C₁₋₆ alkylene-SH. In some embodiments, R^{A} is H. In some embodiments, L⁰ is —(C₁₋₃ alkylene)-O—(C₁₋₃ alkylene)-NR^{B}. In some embodiments, L⁰ is —(C₁₋₆ alkylene)-O—(C₁₋₆ alkylene)-N(H)(C₁₋₆ alkyl). In some embodiments, R^{A} is C₁₋₆ alkyl. In some embodiments, L⁰ is —(C₁₋₆ alkylene)-O—(C₁₋₆ alkylene)-N(C₁₋₆ alkyl)(C₁₋₆ alkyl). In some embodiments, R^{B} is C₁₋₃ alkyl. In some embodiments, R^{B} is —CH₃ or —CH(CH₃)₂. In some embodiments, L⁰ is —CH₂OCH₂CH₂NHCH₃ or —CH₂OCH₂CH₂NHCH(CH₃)₂. In some embodiments, R^{A} is C₁₋₃ alkyl. In some embodiments, R^{B} is C₁₋₃ alkyl. In some embodiments, R^{A} and R^{B} are —CH₃. In some embodiments, L⁰ is —CH₂OCH₂CH₂N(CH₃)₂.

In embodiments of the compound of formula (I), (I-A), (I-B), (I-C), (I-D), (I-A-1), (I-A-2), (I-A-3), (I-A-4), (I-B-1), (I-B-2), (I-B-3), (I-B-4), (I-C-1), (I-C-2), (I-C-3), (I-C-4), (I-D-1), (I-D-2), (I-D-3), or (I-D-4), or a pharmaceutically acceptable salt, stereoisomer, or deuterated form thereof, L⁰ is —(C₁₋₆ alkylene)-O—(C₁₋₆ alkylene)-NR^{A}R^{B}. In some embodiments, R^{A} is H, C₁₋₆ alkyl, C₁₋₆ haloalkyl, C₃₋₈ cycloalkyl, C₃₋₈ hydroxycycloalkyl, aryl, or heterocycle. In some embodiments, R^{B} is C₁₋₆ hydroxyalkyl, C₁₋₆ alkoxy, C₁₋₆ alkylene-NH₂, or C₁₋₆ alkylene-SH. In some embodiments, R^{A} is H. In some embodiments, L⁰ is —(C₁₋₃ alkylene)-O—(C₁₋₃ alkylene)-NHR^{B}. In some embodiments, R^{B} is —CH₃ or —CH(CH₃)₂. In some embodiments, L⁰ is —CH₂OCH₂CH₂NHCH₃ or —CH₂OCH₂CH₂NHCH(CH₃)₂.

In embodiments of the compound of formula (I), (I-A), (I-B), (I-C), (I-D), (I-A-1), (I-A-2), (I-A-3), (I-A-4), (I-B-1), (I-B-2), (I-B-3), (I-B-4), (I-C-1), (I-C-2), (I-C-3), (I-C-4), (I-D-1), (I-D-2), (I-D-3), or (I-D-4), or a pharmaceutically acceptable salt, stereoisomer, or deuterated form thereof, L⁰ is —C(O)NR^{A}R^{B}. In some embodiments, R^{A} is H, C₁₋₆ alkyl, C₁₋₆ haloalkyl, C₃₋₈ cycloalkyl, C₃₋₈ hydroxycycloalkyl, aryl, or heterocycle. In some embodiments, R^{B} is C₁₋₆ alkyl, C₁₋₆ hydroxyalkyl, C₃₋₈ hydroxycycloalkyl, C₁₋₆ alkoxy, C₁₋₆ alkylene-NH₂, or C₁₋₆ alkylene-SH. In some embodiments, R^{B} is C₁₋₆ hydroxyalkyl, C₁₋₆ alkoxy, C₁₋₆ alkylene-NH₂, or C₁₋₆ alkylene-SH. In some embodiments, R^{A} is H. In some embodiments, R^{B} is C₁₋₆ hydroxyalkyl. In some embodiments, L⁰ is —C(O)NHCH₂CH₂OH.

In embodiments of the compound of formula (I), (I-A), (I-B), (I-C), (I-D), (I-A-1), (I-A-2), (I-A-3), (I-A-4), (I-B-1), (I-B-2), (I-B-3), (I-B-4), (I-C-1), (I-C-2), (I-C-3), (I-C-4), (I-D-1), (I-D-2), (I-D-3), or (I-D-4), or a pharmaceutically acceptable salt, stereoisomer, or deuterated form thereof, L⁰ is —NR^{A}C(O)R^{B}. In some embodiments, R^{B} is C₁₋₆ alkyl, C₁₋₆ hydroxyalkyl, C₃₋₈ cycloalkyl, C₁₋₆ alkoxy, C₁₋₆ alkylene-NH₂, or C₁₋₆ alkylene-SH. In some embodiments, R^{B} is C₁₋₆ hydroxyalkyl, and L⁰ is —NR^{A}C(O)C₁₋₆ hydroxyalkyl. In embodiments of the compound of formula (I), (I-A), (I-B), (I-C), (I-D), (I-A-1), (I-A-2), (I-A-3), (I-A-4), (I-B-1), (I-B-2), (I-B-3), (I-B-4), (I-C-1), (I-C-2), (I-C-3), (I-C-4), (I-D-1), (I-D-2), (I-D-3), or (I-D-4), or a pharmaceutically acceptable salt, stereoisomer, or deuterated form thereof, L⁰ is —NR^{A}C(O)C₁₋₆ hydroxyalkyl. In some embodiments, R^{A} is H, C₁₋₆ alkyl, C₁₋₆ haloalkyl, C₃₋₈ cycloalkyl, C₃₋₈ hydroxycycloalkyl, aryl, or heterocycle. In some embodiments, R^{A} is H. In some embodiments, L⁰ is —NHC(O)C₁₋₃ hydroxyalkyl. In some embodiments, L⁰ is —NHC(O)CH₂CH₂OH or —NHC(O)CH₂OH.

In embodiments of the compound of formula (I), (I-A), (I-B), (I-C), (I-D), (I-A-1), (I-A-2), (I-A-3), (I-A-4), (I-B-1), (I-B-2), (I-B-3), (I-B-4), (I-C-1), (I-C-2), (I-C-3), (I-C-4), (I-D-1), (I-D-2), (I-D-3), or (I-D-4), or a pharmaceutically acceptable salt, stereoisomer, or deuterated form thereof, L⁰ is —C₁₋₆ alkylene-NR^{A}C(O)NR^{A}R^{B}. In some embodiments, each R^{A} is independently H, C₁₋₆ alkyl, C₁₋₆ haloalkyl, C₃₋₈ cycloalkyl, C₃₋₈ hydroxycycloalkyl, aryl, or heterocycle. In some embodiments, at least one R^{A} is H. In some embodiments, L⁰ is —C₁₋₃ alkylene-NHC(O)NR^{B}. In some embodiments, R^{B} is C₁₋₆ alkyl, C₁₋₆ hydroxyalkyl, C₃₋₈ cycloalkyl, C₁₋₆ alkoxy, C₁₋₆ alkylene-NH₂, or C₁₋₆ alkylene-SH. In some embodiments, R^{B} is C₁₋₆ hydroxyalkyl. In some embodiments, L⁰ is —CH₂NHC(O)NHCH₂CH₂OH.

In embodiments of the compound of formula (I), (I-A), (I-B), (I-C), (I-D), (I-A-1), (I-A-2), (I-A-3), (I-A-4), (I-B-1), (I-B-2), (I-B-3), (I-B-4), (I-C-1), (I-C-2), (I-C-3), (I-C-4), (I-D-1), (I-D-2), (I-D-3), or (I-D-4), or a pharmaceutically acceptable salt, stereoisomer, or deuterated form thereof, L⁰ is —C₁₋₆ alkylene-NR^{A}C(O)NR^{A}R^{B}. In some embodiments, L⁰ is —C₁₋₃ alkylene-NHC(O)NHR^{B}. In some embodiments, L⁰ is —CH₂NHC(O)NHCH₂CH₂OH.

In embodiments of the compound of formula (I), (I-A), (I-B), (I-C), (I-D), (I-A-1), (I-A-2), (I-A-3), (I-A-4), (I-B-1), (I-B-2), (I-B-3), (I-B-4), (I-C-1), (I-C-2), (I-C-3), (I-C-4), (I-D-1), (I-D-2), (I-D-3), or (I-D-4), or a pharmaceutically acceptable salt, stereoisomer, or deuterated form thereof, L⁰ is —C₃₋₈ cycloalkylene-NR^{A}R^{B}. In some embodiments, R^{A} is H, C₁. 6 alkyl, C₁₋₆ haloalkyl, C₃₋₈ cycloalkyl, C₃₋₈ hydroxycycloalkyl, aryl, or heterocycle. In some embodiments, R^{B} is C₁₋₆ alkyl, C₁₋₆ hydroxyalkyl, C₃₋₈ hydroxycycloalkyl, C₁₋₆ alkoxy, C₁₋₆ alkylene-NH₂, or C₁₋₆ alkylene-SH. In some embodiments, R^{B} is C₁₋₆ hydroxyalkyl, C₁₋₆ alkoxy, C₁₋₆ alkylene-NH₂, or C₁₋₆ alkylene-SH. In some embodiments, R^{A} is H. In some embodiments, L⁰ is —C₃₋₅ cycloalkylene-NHR^{B}. In some embodiments, R^{B} is —CH₂CH₂OH. In some embodiments, L⁰ is

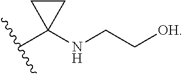 [SMILES: *C1(NCCO)CC1]

In embodiments of the compound of formula (I), (I-A), (I-B), (I-C), (I-D), (I-A-1), (I-A-2), (I-A-3), (I-A-4), (I-B-1), (I-B-2), (I-B-3), (I-B-4), (I-C-1), (I-C-2), (I-C-3), (I-C-4), (I-D-1), (I-D-2), (I-D-3), or (I-D-4), or a pharmaceutically acceptable salt, stereoisomer, or deuterated form thereof, L⁰ is —NR^{A}C(O)NR^{A}R^{B}. In some embodiments, each R^{A} is independently H, C₁₋₆ alkyl, C₁₋₆ haloalkyl, C₃₋₈ cycloalkyl, C₃₋₈ hydroxycycloalkyl, aryl, or heterocycle. In some embodiments, R^{B} is C₁₋₆ alkyl, C₁₋₆ hydroxyalkyl, C₃₋₈ hydroxycycloalkyl, C₁₋₆ alkoxy, C₁₋₆ alkylene-NH₂, or C₁₋₆ alkylene-SH. In some embodiments, R^{B} is C₁₋₆ hydroxyalkyl, C₁₋₆ alkoxy, C₁₋₆ alkylene-NH₂, or C₁₋₆ alkylene-SH. In some embodiments, each R^{A} is H. In some embodiments, L⁰ is —NHC(O)NR^{B}. In some embodiments, R^{B} is —CH₂CH₂OH. In some embodiments, L⁰ is —NHC(O)NHCH₂CH₂OH.

In embodiments of the compound of formula (I), (I-A), (I-B), (I-C), (I-D), (I-A-1), (I-A-2), (I-A-3), (I-A-4), (I-B-1), (I-B-2), (I-B-3), (I-B-4), (I-C-1), (I-C-2), (I-C-3), (I-C-4), (I-D-1), (I-D-2), (I-D-3), or (I-D-4), or a pharmaceutically acceptable salt, stereoisomer, or deuterated form thereof, L⁰ is —O—C₁₋₆ hydroxyalkylene-NR^{A}R^{B}. In some embodiments, R^{A} is H, C₁₋₆ alkyl, C₁₋₆ haloalkyl, C₃₋₈ cycloalkyl, C₃₋₈ hydroxycycloalkyl, aryl, or heterocycle. In some embodiments, R^{B} is C₁₋₆ alkyl, C₁₋₆ hydroxyalkyl, C₃₋₈ hydroxycycloalkyl, C₁₋₆ alkoxy, C₁₋₆ alkylene-NH₂, or C₁₋₆ alkylene-SH. In some embodiments, R^{A} is H. In some embodiments, L⁰ is —O—(C₂₋₅ hydroxyalkylene)-NHR^{B}. In some embodiments, R^{B} is C₁₋₃ alkyl. In some embodiments, R^{B} is —CH(CH₃)₂. In some embodiments, L⁰ is —OCH₂CH(OH)CH₂NHCH(CH₃)₂.

In embodiments of the compound of formula (I), (I-A), (I-B), (I-C), (I-D), (I-A-1), (I-A-2), (I-A-3), (I-A-4), (I-B-1), (I-B-2), (I-B-3), (I-B-4), (I-C-1), (I-C-2), (I-C-3), (I-C-4), (I-D-1), (I-D-2), (I-D-3), or (I-D-4), or a pharmaceutically acceptable salt, stereoisomer, or deuterated form thereof, L⁰ is —O—C₁₋₆ hydroxyalkylene-NR^{A}R^{B}. In some embodiments, R^{A} is H, C₁₋₆ alkyl, C₁₋₆ haloalkyl, C₃₋₈ cycloalkyl, C₃₋₈ hydroxycycloalkyl, aryl, or heterocycle. In some embodiments, R^{B} is C₁₋₆ hydroxyalkyl, C₁₋₆ alkoxy, C₁₋₆ alkylene-NH₂, or C₁₋₆ alkylene-SH. In some embodiments, R^{A} is H. In some embodiments, L⁰ is —O—(C₂₋₅ hydroxyalkylene)-NHR^{B}. In some embodiments, R^{B} is —CH(CH₃)₂. In some embodiments, L⁰ is —OCH₂CH(OH)CH₂NHCH(CH₃)₂.

In embodiments of the compound of formula (I), (I-A), (I-B), (I-C), (I-D), (I-A-1), (I-A-2), (I-A-3), (I-A-4), (I-B-1), (I-B-2), (I-B-3), (I-B-4), (I-C-1), (I-C-2), (I-C-3), (I-C-4), (I-D-1), (I-D-2), (I-D-3), or (I-D-4), or a pharmaceutically acceptable salt, stereoisomer, or deuterated form thereof, L⁰ is —O—C₁₋₆ alkylene-C(O)NR^{A}R^{B}. In some embodiments, R^{A} is H, C₁₋₆ alkyl, C₁₋₆ haloalkyl, C₃₋₈ cycloalkyl, C₃₋₈ hydroxycycloalkyl, aryl, or heterocycle. In some embodiments, R^{B} is C₁₋₆ alkyl, C₁₋₆ hydroxyalkyl, C₃₋₈ hydroxycycloalkyl, C₁₋₆ alkoxy, C₁₋₆ alkylene-NH₂, or C₁₋₆ alkylene-SH. In some embodiments, R^{A} is H. In some embodiments, L⁰ is —O—C₁₋₃ alkylene-C(O)NHR^{B}. In some embodiments, L⁰ is —O—(C₁₋₆ alkylene)C(O)N(H)(C₁₋₆ alkyl). In some embodiments, R^{B} is C₁₋₃ alkyl. In some embodiments, R^{B} is —CH₃. In some embodiments, L⁰ is —OCH₂CH₂C(O)NHCH₃.

In embodiments of the compound of formula (I), (I-A), (I-B), (I-C), (I-D), (I-A-1), (I-A-2), (I-A-3), (I-A-4), (I-B-1), (I-B-2), (I-B-3), (I-B-4), (I-C-1), (I-C-2), (I-C-3), (I-C-4), (I-D-1), (I-D-2), (I-D-3), or (I-D-4), or a pharmaceutically acceptable salt, stereoisomer, or deuterated form thereof, L⁰ is —O—C₁₋₆ alkylene-C(O)NR^{A}R^{B}. In some embodiments, R^{A} is H, C₁₋₆ alkyl, C₁₋₆ haloalkyl, C₃₋₈ cycloalkyl, C₃₋₈ hydroxycycloalkyl, aryl, or heterocycle. In some embodiments, R^{B} is C₁₋₆ hydroxyalkyl, C₁₋₆ alkoxy, C₁₋₆ alkylene-NH₂, or C₁₋₆ alkylene-SH. In some embodiments, R^{A} is H. In some embodiments, L⁰ is —O—C₁₋₃ alkylene-C(O)NR^{B}. In some embodiments, R^{B} is —CH₃. In some embodiments, L⁰ is —OCH₂CH₂C(O)NHCH₃.

In embodiments of the compound of formula (I), (I-A), (I-B), (I-C), (I-D), (I-A-1), (I-A-2), (I-A-3), (I-A-4), (I-B-1), (I-B-2), (I-B-3), (I-B-4), (I-C-1), (I-C-2), (I-C-3), (I-C-4), (I-D-1), (I-D-2), (I-D-3), or (I-D-4), or a pharmaceutically acceptable salt, stereoisomer, or deuterated form thereof, L⁰ is —C₁₋₆ alkylene-O—C₁₋₆ alkylene-C(O)NR^{A}R^{B}. In some embodiments, R^{A} is H, C₁₋₆ alkyl, C₁₋₆ haloalkyl, C₃₋₈ cycloalkyl, C₃₋₈ hydroxycycloalkyl, aryl, or heterocycle. In some embodiments, R^{B} is C₁₋₆ alkyl, C₁₋₆ hydroxyalkyl, C₃₋₈ hydroxycycloalkyl, C₁₋₆ alkoxy, C₁₋₆ alkylene-NH₂, or C₁₋₆ alkylene-SH. In some embodiments, R^{A} is H. In some embodiments, R^{B} is C₁₋₆ alkyl. In some embodiments, L⁰ is —(C₁₋₆ alkylene)-O—(C₁₋₆ alkylene)C(O)N(H)(C₁₋₆ alkyl). In some embodiments, L⁰ is —C₁₋₃ alkylene-O—C₁₋₃ alkylene-C(O)NR^{B}. In some embodiments, R^{B} is C₁₋₃ alkyl. In some embodiments, R^{B} is —CH₃. In some embodiments, L⁰ is —CH₂OCH₂CH₂C(O)NHCH₃.

In embodiments of the compound of formula (I), (I-A), (I-B), (I-C), (I-D), (I-A-1), (I-A-2), (I-A-3), (I-A-4), (I-B-1), (I-B-2), (I-B-3), (I-B-4), (I-C-1), (I-C-2), (I-C-3), (I-C-4), (I-D-1), (I-D-2), (I-D-3), or (I-D-4), or a pharmaceutically acceptable salt, stereoisomer, or deuterated form thereof, L⁰ is —C₁₋₆ alkylene-O—C₁₋₆ alkylene-NR^{A}C(O)—R^{B}. In some embodiments, R^{A} is H, C₁₋₆ alkyl, C₁₋₆ haloalkyl, C₃₋₈ cycloalkyl, C₃₋₈ hydroxycycloalkyl, aryl, or heterocycle. In some embodiments, R^{B} is C₁₋₆ alkyl, C₁₋₆ hydroxyalkyl, C₃₋₈ hydroxycycloalkyl, C₁₋₆ alkoxy, C₁₋₆ alkylene-NH₂, or C₁₋₆ alkylene-SH. In some embodiments, R^{A} is H. In some embodiments, R^{B} is C₁₋₆ alkyl. In some embodiments, L⁰ is —(C₁₋₆ alkylene)-O—(C₁₋₆ alkylene)N(H)C(O)(C₁₋₆ alkyl). In some embodiments, L⁰ is —C₁₋₃ alkylene-O—C₁₋₃ alkylene-NHC(O)—R^{B}. In some embodiments, R^{B} is C₁₋₃ alkyl. In some embodiments, R^{B} is —CH₃. In some embodiments, L⁰ is —CH₂OCH₂CH₂NHC(O)CH₃.

In embodiments of the compound of formula (I), (I-A), (I-B), (I-C), (I-D), (I-A-1), (I-A-2), (I-A-3), (I-A-4), (I-B-1), (I-B-2), (I-B-3), (I-B-4), (I-C-1), (I-C-2), (I-C-3), (I-C-4), (I-D-1), (I-D-2), (I-D-3), or (I-D-4), or a pharmaceutically acceptable salt, stereoisomer, or deuterated form thereof, L⁰ is —C₁₋₆ alkylene-NR^{A}C(O)—(C₁₋₆ hydroxyalkyl). In some embodiments, R^{A} is H, C₁₋₆ alkyl, C₁₋₆ haloalkyl, C₃₋₈ cycloalkyl, C₃₋₈ hydroxycycloalkyl, aryl, or heterocycle. In some embodiments, R^{A} is H. In some embodiments, L⁰ is —(C₁₋₃ alkylene)-NHC(O)—(C₁₋₃ hydroxyalkyl). In some embodiments, L⁰ is —CH₂NHC(O)CH₂OH, or —CH₂NHC(O)CH₂CH₂OH.

In embodiments of the compound of formula (I), (I-A), (I-B), (I-C), (I-D), (I-A-1), (I-A-2), (I-A-3), (I-A-4), (I-B-1), (I-B-2), (I-B-3), (I-B-4), (I-C-1), (I-C-2), (I-C-3), (I-C-4), (I-D-1), (I-D-2), (I-D-3), or (I-D-4), or a pharmaceutically acceptable salt, stereoisomer, or deuterated form thereof, L⁰ is —NR^{A}C(O)NR^{A}—C₁₋₆ alkylene-C₁₋₆ alkoxy. In some embodiments, each R^{A} is independently H, C₁₋₆ alkyl, C₁₋₆ haloalkyl, C₃₋₈ cycloalkyl, C₃₋₈ hydroxycycloalkyl, aryl, or heterocycle. In some embodiments, each R^{A} is H. In some embodiments, L⁰ is —NHC(O)NH—(C₁₋₃ alkylene)-(C₁₋₃ alkoxy). In some embodiments, L⁰ is —NHC(O)NHCH₂CH₂OCH₃.

In embodiments of the compound of formula (I), (I-A), (I-B), (I-C), (I-D), (I-A-1), (I-A-2), (I-A-3), (I-A-4), (I-B-1), (I-B-2), (I-B-3), (I-B-4), (I-C-1), (I-C-2), (I-C-3), (I-C-4), (I-D-1), (I-D-2), (I-D-3), or (I-D-4), or a pharmaceutically acceptable salt, stereoisomer, or deuterated form thereof, L⁰ is —C₁₋₆ alkylene-heterocyclylene-O—C₁₋₆ alkyl. In some embodiments, L⁰ is —C₁₋₃ alkylene-heterocyclylene-O—C₁₋₃ alkyl. In some embodiments, the heterocyclylene is 3-8 membered heterocycle containing 1 or 2 heteroatoms selected from N, O, or S. In some embodiments, the heterocyclylene is 4-6 membered heterocycle containing 1 or 2 heteroatoms selected from N, or O. In embodiments, the heterocyclene is azetidinyl, pyrrolidinyl, piperidinyl, piperazinyl, or oxetanyl. In some embodiments, the heterocyclyene is optionally substituted. In some embodiments, the heterocyclylene is

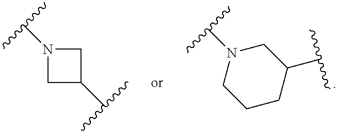 [SMILES: *C1CN(*)C1 ; *C1CCCN(*)C1]

In some embodiments, L⁰ is or

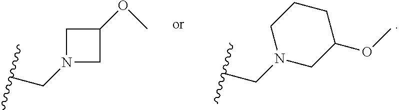

In embodiments of the compound of formula (I), (I-A), (I-B), (I-C), (I-D), (I-A-1), (I-A-2), (I-A-3), (I-A-4), (I-B-1), (I-B-2), (I-B-3), (I-B-4), (I-C-1), (I-C-2), (I-C-3), (I-C-4), (I-D-1), (I-D-2), (I-D-3), or (I-D-4), or a pharmaceutically acceptable salt, stereoisomer, or deuterated form thereof, L⁰ is —C₁₋₆ alkylene-NR^{A}—C₃₋₈ cycloalkylene-R^{B}. In some embodiments, R^{A} is H, C₁₋₆ alkyl, C₁₋₆ haloalkyl, C₃₋₈ cycloalkyl, C₃₋₈ hydroxycycloalkyl, aryl, or heterocycle. In some embodiments, R^{B} is C₁₋₆ alkyl, C₁₋₆ hydroxyalkyl, C₃₋₈ hydroxycycloalkyl, C₁₋₆ alkoxy, C₁₋₆ alkylene-NH₂, or C₁₋₆ alkylene-SH. In some embodiments, R^{A} is H. In some embodiments, R^{B} is C₁₋₆ alkoxy. In some embodiments, L⁰ is —C₁₋₆ alkylene-NH—C₃₋₈ cycloalkylene-R^{B}. In some embodiments, L⁰ is —C₁₋₃ alkylene-NH—C₃₋₈ cycloalkylene-R^{B}. In some embodiments, R^{B} is C₁₋₃ alkoxy. In some embodiments, R^{B} is —OCH₃. In some embodiments, the C₃₋₈ cycloalkylene is

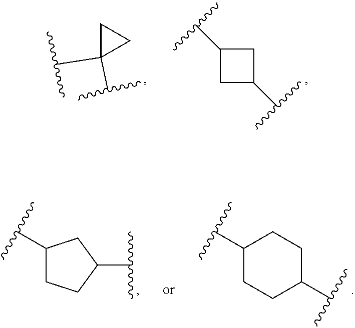 [SMILES: *C1(*)CC1 ; *C1CC(*)C1 ; *C1CCC(*)C1 ; *C1CCC(*)CC1]

In some embodiments, the C₃₋₈ cycloalkylene is

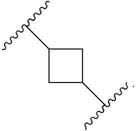 [SMILES: *C1CC(*)C1]

In some embodiments, L⁰ is.

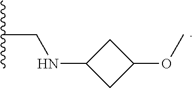 [SMILES: *CNC1CC(OC)C1]

In embodiments of the compound of formula (f), (I-A), (I-B), (I-C), (I-D), (I-A-1), (I-1), (I-D-2), (I-D-3), or (I-D-4), or a pharmaceutically acceptable salt, stereoisomer, or deuterated form thereof, L⁰ is
—CH₂NHCH₂CH₂OH,
—CH₂NHCH₂CH(CH₃)OH,
—CH(CH₃)NHCH₂CH₂OH,

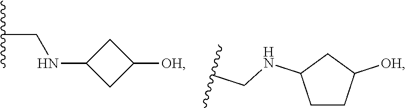 [SMILES: *CNC1CC(O)C1 ; *CNC1CCC(O)C1]

—CH₂NHCH₂CH₂OCH₃,
—CH₂NHCH₂CH₂OCH(CH₃)₂,

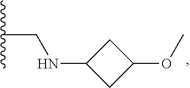 [SMILES: *CNC1CC(OC)C1]

—CH₂OCH₂CH₂NHCH₃,
—CH₂OCH₂CH₂NHCH(CH₃)₂,
—CH₂OCH₂CH₂N(CH₃)₂,
—C(O)NHCH₂CH₂OH,
—NHC(O)CH₂CH₂OH,
—NHC(O)CH₂OH,
—CH₂NHC(O)NHCH₂CH₂OH,

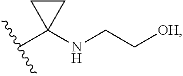 [SMILES: *C1(NCCO)CC1]

—NHC(O)NHCH₂CH₂OH,
—OCH₂CH(OH)CH₂NHCH(CH₃)₂,
—OCH₂CH₂C(O)NHCH₃,
—CH₂OCH₂CH₂C(O)NHCH₃,
—CH₂OCH₂CH₂NHC(O)CH₃,
—CH₂NHC(O)CH₂OH,
—CH₂NHC(O)CH₂CH₂OH, or
—NHC(O)NHCH₂CH₂OCH₃,

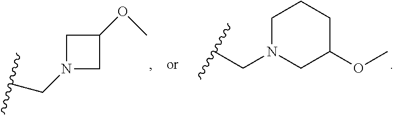

In embodiments of the compound of formula (I), (I-A), (I-B), (I-C), (I-D), (I-A-1), (I-A-2), (I-A-3), (I-A-4), (I-B-1), (I-B-2), (I-B-3), (I-B-4), (I-C-1), (I-C-2), (I-C-3), (I-C-4), (I-D-1), (I-D-2), (I-D-3), or (I-D-4), or a pharmaceutically acceptable salt, stereoisomer, or deuterated form thereof, L⁰ is
—CH₂NHCH₂CH₂OH,
—CH₂NHCH₂CH(CH₃)OH,
—CH(CH₃)NHCH₂CH₂OH,

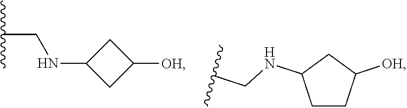 [SMILES: *CNC1CC(O)C1 ; *CNC1CCC(O)C1]

—CH₂NHCH₂CH₂OCH(CH₃)₂,
—CH₂OCH₂CH₂NHCH₃,
—CH₂OCH₂CH₂NHCH(CH₃)₂,
—C(O)NHCH₂CH₂OH,
—NHC(O)CH₂CH₂OH,
—NHC(O)CH₂OH,
—CH₂NHC(O)NHCH₂CH₂OH,

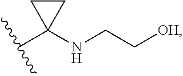 [SMILES: *C1(NCCO)CC1]

—NHC(O)NHCH₂CH₂OH,
—OCH₂CH(OH)CH₂NHCH(CH₃)₂,
—OCH₂CH₂C(O)NHCH₃,
—CH₂NHC(O)CH₂OH,
—CH₂NHC(O)CH₂CH₂OH, or
—NHC(O)NHCH₂CH₂OCH₃.

In embodiments of the compound of formula (I), (I-A), (I-B), (I-C), (I-D), (I-A-1), (I-A-2), (I-A-3), (I-A-4), (I-B-1), (I-B-2), (I-B-3), (I-B-4), (I-C-1), (I-C-2), (I-C-3), (I-C-4), (I-D-1), (I-D-2), (I-D-3), or (I-D-4), or a pharmaceutically acceptable salt, stereoisomer, or deuterated form thereof, L⁰ is —CH₂NHCH₂CH₂OH, —CH₂NHCH₂CH(CH₃)OH, —CH₂OCH₂CH₂NHCH₃, —OCH₂CH(OH)CH₂NHCH(CH₃)₂, —CH₂OCH₂CH₂NHCH(CH₃)₂, or

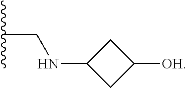 [SMILES: *CNC1CC(O)C1]

In embodiments of the compound of formula (I), (I-A), (I-B), (I-C), (I-D), (I-A-1), (I-A-2), (I-A-3), (I-A-4), (I-B-1), (I-B-2), (I-B-3), (I-B-4), (I-C-1), (I-C-2), (I-C-3), (I-C-4), (I-D-1), (I-D-2), (I-D-3), or (I-D-4), or a pharmaceutically acceptable salt, stereoisomer, or deuterated form thereof, L⁰ is —CH₂NHCH₂CH₂OH.

In embodiments, the present disclosure provides a compound of formula (I-A-1-a), (I-A-2-a), or (I-A-3-a):

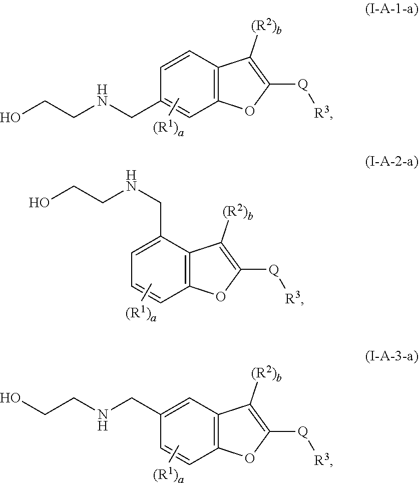

or a pharmaceutically acceptable salt, a stereoisomer, or a deuterated form thereof, wherein:
each R¹ is independently C₁₋₆ alkyl, C₃-C₈ cycloalkyl, or halogen;
each R² is independently C₁₋₆ alkyl, C₃-C₈ cycloalkyl, or halogen;
Q is absent, C₁₋₆ alkylene, —C₁₋₆ alkylene-C(O)—, C₃₋₈ cycloalkylene, —C₃₋₈ cycloalkylene-C(O)—, —C(O)—, or —S(O)₂—;
R³ is carbocycle, heterocycle, aryl, heteroaryl, or —NR^{X}R^{Y}, wherein the carbocycle, heterocycle, aryl, and heteroaryl are each optionally substituted with 1, 2, or 3 R⁴;
each R⁴ is independently halogen, —OH, C₁₋₆ alkyl, C₁₋₆ alkoxy, —C(O)—NR^{X}R^{Y}, or —C(O)O—(C₁₋₆ alkyl);
R^{X} is H, C₁₋₆ alkyl, C₁₋₆ haloalkyl, or C₁₋₆ hydroxyalkyl;
R^{Y} is C₁₋₆ alkyl, C₃₋₈ cycloalkyl, C₃₋₈ hydroxycycloalkyl, aryl, or heterocycle;
a is an integer of 0-3; and
b is an integer of 0 or 1.

In embodiments, the present disclosure provides a compound of formula (I-B-1-a), (I-B-2-a), or (I-B-3-a):

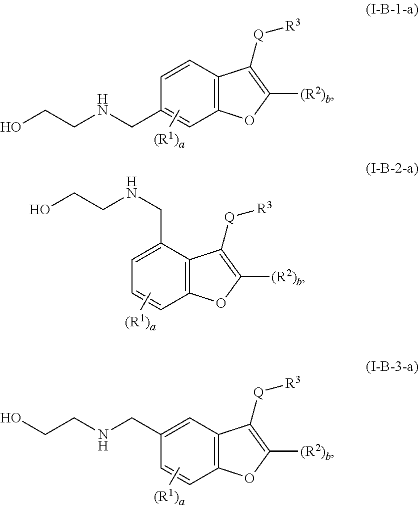

or a pharmaceutically acceptable salt, a stereoisomer, or a deuterated form thereof,
wherein:
each R¹ is independently C₁₋₆ alkyl, C₃-C₈ cycloalkyl, or halogen;
each R² is independently C₁₋₆ alkyl, C₃-C₈ cycloalkyl, or halogen;
Q is absent, C₁₋₆ alkylene, —C₁₋₆ alkylene-C(O)—, C₃₋₈ cycloalkylene, —C₃₋₈ cycloalkylene-C(O)—, —C(O)—, or —S(O)₂—;
R³ is carbocycle, heterocycle, aryl, heteroaryl, or —NR^{X}R^{Y}, wherein the carbocycle, heterocycle, aryl, and heteroaryl are each optionally substituted with 1, 2, or 3 R⁴;
each R⁴ is independently halogen, —OH, C₁₋₆ alkyl, C₁₋₆ alkoxy, —C(O)—NR^{X}R^{Y}, or —C(O)O—(C₁₋₆ alkyl);
R^{X} is H, C₁₋₆ alkyl, C₁₋₆ haloalkyl, or C₁₋₆ hydroxyalkyl;
R^{Y} is C₁₋₆ alkyl, C₃₋₈ cycloalkyl, C₃₋₈ hydroxycycloalkyl, aryl, or heterocycle;
a is an integer of 0-3; and
b is an integer of 0 or 1.

In embodiments, the present disclosure provides a compound of formula (I-C-1-a) or (I-D-1-a):

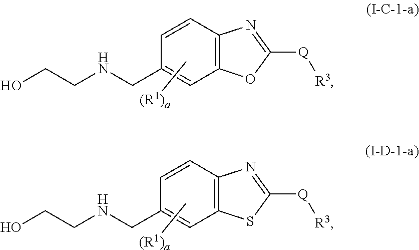

or a pharmaceutically acceptable salt, a stereoisomer, or a deuterated form thereof,
wherein:
each R¹ is independently C₁₋₆ alkyl, C₃-C₈ cycloalkyl, or halogen;
Q is absent, C₁₋₆ alkylene, —C₁₋₆ alkylene-C(O)—, C₃₋₈ cycloalkylene, —C₃₋₈ cycloalkylene-C(O)—, —C(O)—, or —S(O)₂—;
R³ is carbocycle, heterocycle, aryl, heteroaryl, or —NR^{X}R^{Y}, wherein the carbocycle, heterocycle, aryl, and heteroaryl are each optionally substituted with 1, 2, or 3 R⁴;
each R⁴ is independently halogen, —OH, C₁₋₆ alkyl, C₁₋₆ alkoxy, —C(O)—NR^{X}R^{Y}, or —C(O)O—(C₁₋₆ alkyl);
each R^{A} is independently H, C₁₋₆ alkyl, C₁₋₆ haloalkyl, C₃₋₈ cycloalkyl, C₃₋₈ hydroxycycloalkyl, aryl, or heterocycle;
each R^{B} is independently C₁₋₆ hydroxyalkyl, C₁₋₆ alkoxy, C₁₋₆ alkylene-NH₂, or C₁₋₆ alkylene-SH;
R^{X} is H, C₁₋₆ alkyl, C₁₋₆ haloalkyl, or C₁₋₆ hydroxyalkyl;
R^{Y} is C₁₋₆ alkyl, C₃₋₈ cycloalkyl, C₃₋₈ hydroxycycloalkyl, aryl, or heterocycle; and
a is an integer of 0-3.

In embodiments of the compound of formula (I), (I-A), (I-B), (I-C), (I-D), (I-A-1), (I-A-2), (I-A-3), (I-A-4), (I-B-1), (I-B-2), (I-B-3), (I-B-4), (I-C-1), (I-C-2), (I-C-3), (I-C-4), (I-D-1), (I-D-2), (I-D-3), (I-D-4), (I-A-1-a), (I-A-2-a), (I-A-3-a), (I-B-1-a), (I-B-2-a), (I-B-3-a), (I-C-1-a), or (I-D-1-a), or a pharmaceutically acceptable salt, stereoisomer, or deuterated form thereof, at least one H in L⁰ is replaced by conjugate comprising a ligand that binds to a protein, a protein aggregate, a protein complex, or a lipid.

In embodiments, the present disclosure provides for components in which a linker is covalently attached to the compounds of (X-I), (I), (I-A), (I-B), (I-C), (I-D), (I-A-1), (I-A-2), (I-A-3), (I-A-4), (I-B-1), (I-B-2), (I-B-3), (I-B-4), (I-C-1), (I-C-2), (I-C-3), (I-C-4), (I-D-1), (I-D-2), (I-D-3), (I-D-4), (I-A-1-a), (I-A-2-a), (I-A-3-a), (I-B-1-a), (I-B-2-a), (I-B-3-a), (I-C-1-a). The linker (e.g., L¹ disclosed herein) may be any moiety that is capable of covalently binding to the warhead and to the protein binding component (PBC). In embodiments, the present disclosure provides a compound of formula (X-II):

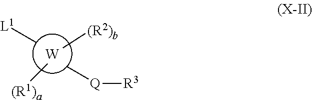

or a pharmaceutically acceptable salt, stereoisomer, or deuterated form thereof,
wherein:
ring W is 6,5-fused heteroaryl or 6,5-fused heterocycle ring, wherein each ring W contains 1, 2, or 3 heteroatoms selected from N, O, or S, and at least 1 of the heteroatoms is N or O;
L¹ is C₂-C₅₀ alkylene-R⁵, C₂-C₂₅ alkenylene-R⁵, C₂-C₂₅ alkynylene-R⁵, wherein 1-25 methylene groups of L¹ are optionally and independently replaced by —N(H)—, —N(C₁-C₆ alkyl)-, —N(C₃-C₈ cycloalkyl)-, —O—, —C(O)—, —C(O)O—, —S—, —S(O)—, —S(O)₂—, —S(O)₂N(C₁-C₆ alkyl)-, —S(O)₂N(C₃-C₈ cycloalkyl)-, —N(H)C(O)—, —N(C₁-C₆ alkyl)C(O)—, —N(C₃-C₈ cycloalkyl)C(O)—, —N(H)C(O)N(H)—, —N(C₁-C₆ alkyl)C(O)N(H)—, —N(H)C(O)N(C₁-C₆ alkyl)-, —N(C₁-C₆ alkyl)C(O)N(C₁-C₆ alkyl)-, —C(O)N(H)—, —C(O)N(C₁-C₆ alkyl)-, —C(O)N(C₃-C₈ cycloalkyl)-, arylene, heteroarylene, heterocyclylene, C₃-C₈ cycloalkylene, or C₃-C₈ cycloalkenylene,
wherein the alkylene, alkenylene, alkynylene, alkyl, arylene, heteroarylene, and heterocyclylene are each optionally and independently substituted with 1, 2, or 3 R^{Z}, wherein
each R^{Z} is independently —OH, C₁₋₆ alkyl, or halogen;
each R¹ is independently C₁₋₆ alkyl, C₃-C₈ cycloalkyl, or halogen;
each R² is independently C₁₋₆ alkyl, C₃-C₈ cycloalkyl, or halogen, or two R² form an oxo;
Q is absent, C₁₋₆ alkylene, —C₁₋₆ alkylene-C(O)—, C₃₋₈ cycloalkylene, —C₃₋₈ cycloalkylene-C(O)—, —C(O)—, or —S(O)₂—;
R³ is carbocycle, heterocycle, aryl, heteroaryl, or —NR^{X}R^{Y}, wherein the carbocycle, heterocycle, aryl, and heteroaryl are each optionally substituted with 1, 2, or 3 R⁴;
each R⁴ is independently halogen, —OH, C₁₋₆ alkyl, C₁₋₆ alkoxy, —C(O)—NR^{X}R^{Y}, or —C(O)O—(C₁₋₆ alkyl);
each R⁵ is independently a bond, a leaving group, a protecting group, H, alkynyl, aryl, or heteroaryl;
R^{X} is H, C₁₋₆ alkyl, C₁₋₆ haloalkyl, or C₁₋₆ hydroxyalkyl;
R^{Y} is C₁₋₆ alkyl, C₃₋₈ cycloalkyl, C₃₋₈ hydroxycycloalkyl, aryl, or heterocycle;
a is an integer of 0-3; and
b is an integer of 0-2.

In embodiments, the present disclosure provides a compound of formula (II)

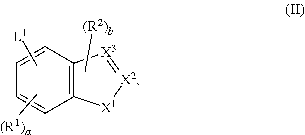

or a pharmaceutically acceptable salt, stereoisomer, or deuterated form thereof,
wherein:
L¹ is C₂-C₅₀ alkylene-R⁵, C₂-C₂₅ alkenylene-R⁵, C₂-C₂₅ alkynylene-R⁵, wherein 1-25 methylene groups of L¹ are optionally and independently replaced by —N(H)—, —N(C₁-C₆ alkyl)-, —N(C₃-C₈ cycloalkyl)-, —O—, —C(O)—, —C(O)O—, —S—, —S(O)—, —S(O)₂—, —S(O)₂N(C₁-C₆ alkyl)-, —S(O)₂N(C₃-C₈ cycloalkyl)-, —N(H)C(O)—, —N(C₁-C₆ alkyl)C(O)—, —N(C₃-C₈ cycloalkyl)C(O)—, —N(H)C(O)N(H)—, —N(C₁-C₆ alkyl)C(O)N(H)—, —N(H)C(O)N(C₁-C₆ alkyl)-, —N(C₁-C₆ alkyl)C(O)N(C₁-C₆ alkyl)-, —C(O)N(H)—, —C(O)N(C₁-C₆ alkyl)-, —C(O)N(C₃-C₈ cycloalkyl)-, arylene, heteroarylene, heterocyclylene, C₃-C₈ cycloalkylene, or C₃-C₈ cycloalkenylene, wherein the alkylene, alkenylene, alkynylene, alkyl, arylene, heteroarylene, and heterocyclylene are each optionally and independently substituted with 1, 2, or 3 R^{Z}, wherein each R^{Z} is independently —OH, C₁₋₆ alkyl, or halogen;
each R¹ is independently C₁₋₆ alkyl, C₃-C₈ cycloalkyl, or halogen;
each R² is independently C₁₋₆ alkyl, C₃-C₈ cycloalkyl, or halogen;
X¹ is O or S;
one of X² or X³ is C-Q-R³ and the other is CH, C, or N as permitted by valency;
Q is absent, C₁₋₆ alkylene, —C₁₋₆ alkylene-C(O)—, C₃₋₈ cycloalkylene, —C₃₋₈ cycloalkylene-C(O)—, —C(O)—, or —S(O)₂—;
R³ is carbocycle, heterocycle, aryl, heteroaryl, or —NR^{X}R^{Y}, wherein the carbocycle, heterocycle, aryl, and heteroaryl are each optionally substituted with 1, 2, or 3 R⁴;
each R⁴ is independently halogen, —OH, C₁₋₆ alkyl, C₁₋₆ alkoxy, —C(O)—NR^{X}R^{Y}, or —C(O)O—(C₁₋₆ alkyl);
each R⁵ is independently a bond, a leaving group, a protecting group, H, alkynyl, aryl, or heteroaryl;
R^{X} is H, C₁₋₆ alkyl, C₁₋₆ haloalkyl, or C₁₋₆ hydroxyalkyl;
R^{Y} is C₁₋₆ alkyl, C₃₋₈ cycloalkyl, C₃₋₈ hydroxycycloalkyl, aryl, or heterocycle;
a is an integer of 0-3; and
b is an integer of 0 or 1.

In embodiments, the present disclosure provides a compound of formula (II-A):

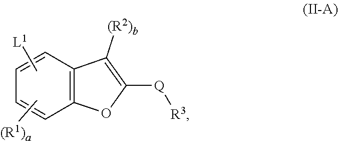

or a pharmaceutically acceptable salt, a stereoisomer, or a deuterated form thereof,
wherein:
L¹ is C₂-C₅₀ alkylene-R⁵, C₂-C₂₅ alkenylene-R⁵, C₂-C₂₅ alkynylene-R⁵, wherein 1-25 methylene groups of L¹ are optionally and independently replaced by —N(H)—, —N(C₁-C₆ alkyl)-, —N(C₃-C₈ cycloalkyl)-, —O—, —C(O)—, —C(O)O—, —S—, —S(O)—, —S(O)₂—, —S(O)₂N(C₁-C₆ alkyl)-, —S(O)₂N(C₃-C₈ cycloalkyl)-, —N(H)C(O)—, —N(C₁-C₆ alkyl)C(O)—, —N(C₃-C₈ cycloalkyl)C(O)—, —N(H)C(O)N(H)—, —N(C₁-C₆ alkyl)C(O)N(H)—, —N(H)C(O)N(C₁-C₆ alkyl)-, —N(C₁-C₆ alkyl)C(O)N(C₁-C₆ alkyl)-, —C(O)N(H)—, —C(O)N(C₁-C₆ alkyl)-, —C(O)N(C₃-C₈ cycloalkyl)-, arylene, heteroarylene, heterocyclylene, C₃-C₈ cycloalkylene, or C₃-C₈ cycloalkenylene, wherein the alkylene, alkenylene, alkynylene, alkyl, arylene, heteroarylene, and heterocyclylene are each optionally and independently substituted with 1, 2, or 3 R^{Z}, wherein each R^{Z} is independently —OH, C₁₋₆ alkyl, or halogen;
each R¹ is independently C₁₋₆ alkyl, C₃-C₈ cycloalkyl, or halogen;
each R² is independently C₁₋₆ alkyl, C₃-C₈ cycloalkyl, or halogen;
Q is absent, C₁₋₆ alkylene, —C₁₋₆ alkylene-C(O)—, C₃₋₈ cycloalkylene, —C₃₋₈ cycloalkylene-C(O)—, —C(O)—, or —S(O)₂—;
R³ is carbocycle, heterocycle, aryl, heteroaryl, or —NR^{X}R^{Y}, wherein the carbocycle, heterocycle, aryl, and heteroaryl are each optionally substituted with 1, 2, or 3 R⁴;
each R⁴ is independently halogen, —OH, C₁₋₆ alkyl, C₁₋₆ alkoxy, —C(O)—NR^{X}R^{Y}, or —C(O)O—(C₁₋₆ alkyl);
each R⁵ is independently a bond, a leaving group, a protecting group, H, alkynyl, aryl, or heteroaryl;
R^{X} is H, C₁₋₆ alkyl, C₁₋₆ haloalkyl, or C₁₋₆ hydroxyalkyl;
R^{Y} is C₁₋₆ alkyl, C₃₋₈ cycloalkyl, C₃₋₈ hydroxycycloalkyl, aryl, or heterocycle;
a is an integer of 0-3; and
b is an integer of 0 or 1.

In embodiments, the present disclosure provides a compound of formula (II-A-1), (II-A-2), (II-A-3), or (II-A-4):

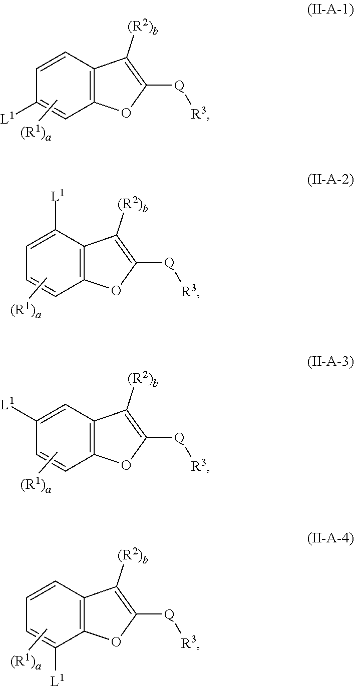

or a pharmaceutically acceptable salt, a stereoisomer, or a deuterated form thereof,
wherein:
L¹ is C₂-C₅₀ alkylene-R⁵, C₂-C₂₅ alkenylene-R⁵, C₂-C₂₅ alkynylene-R⁵, wherein 1-25 methylene groups of L¹ are optionally and independently replaced by —N(H)—, —N(C₁-C₆ alkyl)-, —N(C₃-C₈ cycloalkyl)-, —O—, —C(O)—, —C(O)O—, —S—, —S(O)—, —S(O)₂—, —S(O)₂N(C₁-C₆ alkyl)-, —S(O)₂N(C₃-C₈ cycloalkyl)-, —N(H)C(O)—, —N(C₁-C₆ alkyl)C(O)—, —N(C₃-C₈ cycloalkyl)C(O)—, —N(H)C(O)N(H)—, —N(C₁-C₆ alkyl)C(O)N(H)—, —N(H)C(O)N(C₁-C₆ alkyl)-, —N(C₁-C₆ alkyl)C(O)N(C₁-C₆ alkyl)-, —C(O)N(H)—, —C(O)N(C₁-C₆ alkyl)-, —C(O)N(C₃-C₈ cycloalkyl)-, arylene, heteroarylene, heterocyclylene, C₃-C₈ cycloalkylene, or C₃-C₈ cycloalkenylene, wherein the alkylene, alkenylene, alkynylene, alkyl, arylene, heteroarylene, and heterocyclylene are each optionally and independently substituted with 1, 2, or 3 R^{Z}, wherein each R^{Z} is independently —OH, C₁₋₆ alkyl, or halogen;
each R¹ is independently C₁₋₆ alkyl, C₃-C₈ cycloalkyl, or halogen;
each R² is independently C₁₋₆ alkyl, C₃-C₈ cycloalkyl, or halogen;
Q is absent, C₁₋₆ alkylene, —C₁₋₆ alkylene-C(O)—, C₃₋₈ cycloalkylene, —C₃₋₈ cycloalkylene-C(O)—, —C(O)—, or —S(O)₂—;
R³ is carbocycle, heterocycle, aryl, heteroaryl, or —NR^{X}R^{Y}, wherein the carbocycle, heterocycle, aryl, and heteroaryl are each optionally substituted with 1, 2, or 3 R⁴;
each R⁴ is independently halogen, —OH, C₁₋₆ alkyl, C₁₋₆ alkoxy, —C(O)—NR^{X}R^{Y}, or —C(O)O—(C₁₋₆ alkyl);
each R⁵ is independently a bond, a leaving group, a protecting group, H, alkynyl, aryl, or heteroaryl;
R^{X} is H, C₁₋₆ alkyl, C₁₋₆ haloalkyl, or C₁₋₆ hydroxyalkyl;
R^{Y} is C₁₋₆ alkyl, C₃₋₈ cycloalkyl, C₃₋₈ hydroxycycloalkyl, aryl, or heterocycle;
a is an integer of 0-3; and
b is an integer of 0 or 1.

In embodiments, the present disclosure provides a compound of formula (II-B):

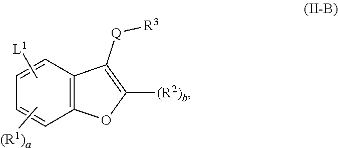

or a pharmaceutically acceptable salt, a stereoisomer, or a deuterated form thereof, wherein:
L¹ is C₂-C₅₀ alkylene-R⁵, C₂-C₂₅ alkenylene-R⁵, C₂-C₂₅ alkynylene-R⁵, wherein 1-25 methylene groups of L¹ are optionally and independently replaced by —N(H)—, —N(C₁-C₆ alkyl)-, —N(C₃-C₈ cycloalkyl)-, —O—, —C(O)—, —C(O)O—, —S—, —S(O)—, —S(O)₂—, —S(O)₂N(C₁-C₆ alkyl)-, —S(O)₂N(C₃-C₈ cycloalkyl)-, —N(H)C(O)—, —N(C₁-C₆ alkyl)C(O)—, —N(C₃-C₈ cycloalkyl)C(O)—, —N(H)C(O)N(H)—, —N(C₁-C₆ alkyl)C(O)N(H)—, —N(H)C(O)N(C₁-C₆ alkyl)-, —N(C₁-C₆ alkyl)C(O)N(C₁-C₆ alkyl)-, —C(O)N(H)—, —C(O)N(C₁-C₆ alkyl)-, —C(O)N(C₃-C₈ cycloalkyl)-, arylene, heteroarylene, heterocyclylene, C₃-C₈ cycloalkylene, or C₃-C₈ cycloalkenylene, wherein the alkylene, alkenylene, alkynylene, alkyl, arylene, heteroarylene, and heterocyclylene are each optionally and independently substituted with 1, 2, or 3 R^{Z}, wherein each R^{Z} is independently —OH, C₁₋₆ alkyl, or halogen;
each R¹ is independently C₁₋₆ alkyl, C₃-C₈ cycloalkyl, or halogen;
each R² is independently C₁₋₆ alkyl, C₃-C₈ cycloalkyl, or halogen;
Q is absent, C₁₋₆ alkylene, —C₁₋₆ alkylene-C(O)—, C₃₋₈ cycloalkylene, —C₃₋₈ cycloalkylene-C(O)—, —C(O)—, or —S(O)₂—;
R³ is carbocycle, heterocycle, aryl, heteroaryl, or —NR^{X}R^{Y}, wherein the carbocycle, heterocycle, aryl, and heteroaryl are each optionally substituted with 1, 2, or 3 R⁴;
each R⁴ is independently halogen, —OH, C₁₋₆ alkyl, C₁₋₆ alkoxy, —C(O)—NR^{X}R^{Y}, or —C(O)O—(C₁₋₆ alkyl);
each R⁵ is independently a bond, a leaving group, a protecting group, H, alkynyl, aryl, or heteroaryl;
R^{X} is H, C₁₋₆ alkyl, C₁₋₆ haloalkyl, or C₁₋₆ hydroxyalkyl;
R^{Y} is C₁₋₆ alkyl, C₃₋₈ cycloalkyl, C₃₋₈ hydroxycycloalkyl, aryl, or heterocycle;
a is an integer of 0-3; and
b is an integer of 0 or 1.

In embodiments, the present disclosure provides a compound of formula (II-B-1), (II-B-2), (II-B-3), or (II-B-4):

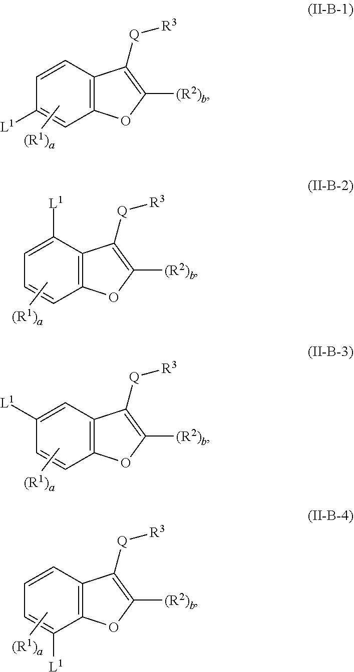

or a pharmaceutically acceptable salt, a stereoisomer, or a deuterated form thereof,
wherein:
L¹ is C₂-C₅₀ alkylene-R⁵, C₂-C₂₅ alkenylene-R⁵, C₂-C₂₅ alkynylene-R⁵, wherein 1-25 methylene groups of L¹ are optionally and independently replaced by —N(H)—, —N(C₁-C₆ alkyl)-, —N(C₃-C₈ cycloalkyl)-, —O—, —C(O)—, —C(O)O—, —S—, —S(O)—, —S(O)₂—, —S(O)₂N(C₁-C₆ alkyl)-, —S(O)₂N(C₃-C₈ cycloalkyl)-, —N(H)C(O)—, —N(C₁-C₆ alkyl)C(O)—, —N(C₃-C₈ cycloalkyl)C(O)—, —N(H)C(O)N(H)—, —N(C₁-C₆ alkyl)C(O)N(H)—, —N(H)C(O)N(C₁-C₆ alkyl)-, —N(C₁-C₆ alkyl)C(O)N(C₁-C₆ alkyl)-, —C(O)N(H)—, —C(O)N(C₁-C₆ alkyl)-, —C(O)N(C₃-C₈ cycloalkyl)-, arylene, heteroarylene, heterocyclylene, C₃-C₈ cycloalkylene, or C₃-C₈ cycloalkenylene, wherein the alkylene, alkenylene, alkynylene, alkyl, arylene, heteroarylene, and heterocyclylene are each optionally and independently substituted with 1, 2, or 3 R^{Z}, wherein each R^{Z} is independently —OH, C₁₋₆ alkyl, or halogen;
each R¹ is independently C₁₋₆ alkyl, C₃-C₈ cycloalkyl, or halogen;
each R² is independently C₁₋₆ alkyl, C₃-C₈ cycloalkyl, or halogen;
Q is absent, C₁₋₆ alkylene, —C₁₋₆ alkylene-C(O)—, C₃₋₈ cycloalkylene, —C₃₋₈ cycloalkylene-C(O)—, —C(O)—, or —S(O)₂—;
R³ is carbocycle, heterocycle, aryl, heteroaryl, or —NR^{X}R^{Y}, wherein the carbocycle, heterocycle, aryl, and heteroaryl are each optionally substituted with 1, 2, or 3 R⁴;
each R⁴ is independently halogen, —OH, C₁₋₆ alkyl, C₁₋₆ alkoxy, —C(O)—NR^{X}R^{Y}, or —C(O)O—(C₁₋₆ alkyl);
each R⁵ is independently a bond, a leaving group, a protecting group, H, alkynyl, aryl, or heteroaryl;
R^{X} is H, C₁₋₆ alkyl, C₁₋₆ haloalkyl, or C₁₋₆ hydroxyalkyl;
R^{Y} is C₁₋₆ alkyl, C₃₋₈ cycloalkyl, C₃₋₈ hydroxycycloalkyl, aryl, or heterocycle;
a is an integer of 0-3; and
b is an integer of 0 or 1.

In embodiments, the present disclosure provides a compound of formula (II-C) or (II-D):

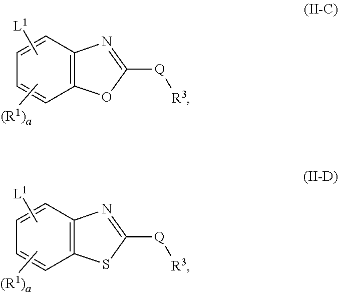

or a pharmaceutically acceptable salt, a stereoisomer, or a deuterated form thereof, wherein:
L¹ is C₂-C₅₀ alkylene-R⁵, C₂-C₂₅ alkenylene-R⁵, C₂-C₂₅ alkynylene-R⁵, wherein 1-25 methylene groups of L¹ are optionally and independently replaced by —N(H)—, —N(C₁-C₆ alkyl)-, —N(C₃-C₈ cycloalkyl)-, —O—, —C(O)—, —C(O)O—, —S—, —S(O)—, —S(O)₂—, —S(O)₂N(C₁-C₆ alkyl)-, —S(O)₂N(C₃-C₈ cycloalkyl)-, —N(H)C(O)—, —N(C₁-C₆ alkyl)C(O)—, —N(C₃-C₈ cycloalkyl)C(O)—, —N(H)C(O)N(H)—, —N(C₁-C₆ alkyl)C(O)N(H)—, —N(H)C(O)N(C₁-C₆ alkyl)-, —N(C₁-C₆ alkyl)C(O)N(C₁-C₆ alkyl)-, —C(O)N(H)—, —C(O)N(C₁-C₆ alkyl)-, —C(O)N(C₃-C₈ cycloalkyl)-, arylene, heteroarylene, heterocyclylene, C₃-C₈ cycloalkylene, or C₃-C₈ cycloalkenylene, wherein the alkylene, alkenylene, alkynylene, alkyl, arylene, heteroarylene, and heterocyclylene are each optionally and independently substituted with 1, 2, or 3 R^{Z}, wherein each R^{Z} is independently —OH, C₁₋₆ alkyl, or halogen;
each R¹ is independently C₁₋₆ alkyl, C₃-C₈ cycloalkyl, or halogen;
Q is absent, C₁₋₆ alkylene, —C₁₋₆ alkylene-C(O)—, C₃₋₈ cycloalkylene, —C₃₋₈ cycloalkylene-C(O)—, —C(O)—, or —S(O)₂—;
R³ is carbocycle, heterocycle, aryl, heteroaryl, or —NR^{X}R^{Y}, wherein the carbocycle, heterocycle, aryl, and heteroaryl are each optionally substituted with 1, 2, or 3 R⁴;
each R⁴ is independently halogen, —OH, C₁₋₆ alkyl, C₁₋₆ alkoxy, —C(O)—NR^{X}R^{Y}, or —C(O)O—(C₁₋₆ alkyl);
each R⁵ is independently a bond, a leaving group, a protecting group, H, alkynyl, aryl, or heteroaryl;
R^{X} is H, C₁₋₆ alkyl, C₁₋₆ haloalkyl, or C₁₋₆ hydroxyalkyl;
R^{Y} is C₁₋₆ alkyl, C₃₋₈ cycloalkyl, C₃₋₈ hydroxycycloalkyl, aryl, or heterocycle; and
a is an integer of 0-3.

In embodiments, the present disclosure provides a compound of formula (II-C-1), (II-C-2), (II-C-3), (II-C-4), (II-D-1), (II-D-2), (II-D-3), or (II-D-4):

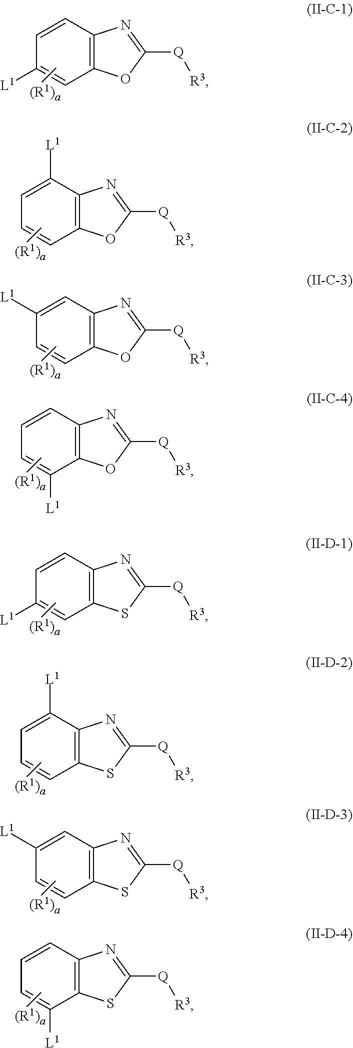

or a pharmaceutically acceptable salt, a stereoisomer, or a deuterated form thereof,
wherein:
L¹ is C₂-C₅₀ alkylene-R⁵, C₂-C₂₅ alkenylene-R⁵, C₂-C₂₅ alkynylene-R⁵, wherein 1-25 methylene groups of L¹ are optionally and independently replaced by —N(H)—, —N(C₁-C₆ alkyl)-, —N(C₃-C₈ cycloalkyl)-, —O—, —C(O)—, —C(O)O—, —S—, —S(O)—, —S(O)₂—, —S(O)₂N(C₁-C₆ alkyl)-, —S(O)₂N(C₃-C₈ cycloalkyl)-, —N(H)C(O)—, —N(C₁-C₆ alkyl)C(O)—, —N(C₃-C₈ cycloalkyl)C(O)—, —N(H)C(O)N(H)—, —N(C₁-C₆ alkyl)C(O)N(H)—, —N(H)C(O)N(C₁-C₆ alkyl)-, —N(C₁-C₆ alkyl)C(O)N(C₁-C₆ alkyl)-, —C(O)N(H)—, —C(O)N(C₁-C₆ alkyl)-, —C(O)N(C₃-C₈ cycloalkyl)-, arylene, heteroarylene, heterocyclylene, C₃-C₈ cycloalkylene, or C₃-C₈ cycloalkenylene, wherein the alkylene, alkenylene, alkynylene, alkyl, arylene, heteroarylene, and heterocyclylene are each optionally and independently substituted with 1, 2, or 3 R^{Z}, wherein each R^{Z} is independently —OH, C₁₋₆ alkyl, or halogen;
each R¹ is independently C₁₋₆ alkyl, C₃-C₈ cycloalkyl, or halogen;
Q is absent, C₁₋₆ alkylene, —C₁₋₆ alkylene-C(O)—, C₃₋₈ cycloalkylene, —C₃₋₈ cycloalkylene-C(O)—, —C(O)—, or —S(O)₂—;
R³ is carbocycle, heterocycle, aryl, heteroaryl, or —NR^{X}R^{Y}, wherein the carbocycle, heterocycle, aryl, and heteroaryl are each optionally substituted with 1, 2, or 3 R⁴;
each R⁴ is independently halogen, —OH, C₁₋₆ alkyl, C₁₋₆ alkoxy, —C(O)—NR^{X}R^{Y}, or —C(O)O—(C₁₋₆ alkyl);
each R⁵ is independently a bond, a leaving group, a protecting group, H, alkynyl, aryl, or heteroaryl;
R^{X} is H, C₁₋₆ alkyl, C₁₋₆ haloalkyl, or C₁₋₆ hydroxyalkyl;
R^{Y} is C₁₋₆ alkyl, C₃₋₈ cycloalkyl, C₃₋₈ hydroxycycloalkyl, aryl, or heterocycle; and
a is an integer of 0-3.

### L¹

In embodiments of the compound of formula (X-II), (II), (II-A), (II-B), (II-C), (II-D), (II-A-1), (II-A-2), (II-A-3), (II-A-4), (II-B3-1), (II-B3-2), (II-B-3), (II-B-4), (II-C-1), (II-C-2), (II-C-3), (II-C-4), (II-D-1), (II-D-2), (II-D-3), or (II-D-4), or a pharmaceutically acceptable salt, stereoisomer, or deuterated form thereof, L¹ is C₂-C₅₀ alkylene-R⁵, C₂-C₂₅ alkenylene-R⁵, C₂-C₂₅ alkynylene-R⁵, wherein 1-25 methylene groups of L¹ are optionally and independently replaced by —N(H)—, —N(C₁-C₆ alkyl)-, —N(C₃-C₈ cycloalkyl)-, —O—, —C(O)—, —C(O)O—, —S—, —S(O)—, —S(O)₂—, —S(O)₂N(C₁-C₆ alkyl)-, —S(O)₂N(C₃-C₈ cycloalkyl)-, —N(H)C(O)—, —N(C₁-C₆ alkyl)C(O)—, —N(C₃-C₈ cycloalkyl)C(O)—, —N(H)C(O)N(H)—, —N(C₁-C₆ alkyl)C(O)N(H)—, —N(H)C(O)N(C₁-C₆ alkyl)-, —N(C₁-C₆ alkyl)C(O)N(C₁-C₆ alkyl)-, —C(O)N(H)—, —C(O)N(C₁-C₆ alkyl)-, —C(O)N(C₃-C₈ cycloalkyl)-, arylene, heteroarylene, heterocyclylene, C₃-C₈ cycloalkylene, or C₃-C₈ cycloalkenylene, wherein the alkylene, alkenylene, alkynylene, alkyl, arylene, heteroarylene, and heterocyclylene are each optionally substituted. In embodiments, the alkylene, alkenylene, alkynylene, alkyl, arylene, heteroarylene, and heterocyclylene are each optionally substituted with 1, 2, or 3 R^{Z}, wherein each R^{Z} is independently —OH, C₁₋₆ alkyl, or halogen. In embodiments, each R⁵ is independently a bond, a leaving group, a protecting group, H, alkynyl, aryl, or heteroaryl.

In embodiments of the compound of formula (II), (II-A), (II-B), (II-C), (II-D), (II-A-1), (II-A-2), (II-A-3), (II-A-4), (II-B3-1), (II-B3-2), (II-B-3), (II-B-4), (II-C-1), (II-C-2), (II-C-3), (II-C-4), (II-D-1), (II-D-2), (II-D-3), or (II-D-4), or a pharmaceutically acceptable salt, stereoisomer, or deuterated form thereof, 1-25 methylene groups of L¹ are optionally and independently replaced by —N(H)—, —N(C₁-C₆ alkyl)-, —N(C₃-C₈ cycloalkyl)-, —O—, —C(O)—, —C(O)O—, —N(H)C(O)—, —N(C₁-C₆ alkyl)C(O)—, —N(C₃-C₈ cycloalkyl)C(O)—, —C(O)N(H)—, —C(O)N(C₁-C₆ alkyl)-, —C(O)N(C₃-C₈ cycloalkyl)-, arylene, heteroarylene, heterocyclylene, or C₃-C₈ cycloalkylene, wherein each arylene or heteroarylene is optionally and independently substituted. In some embodiments, each arylene or heteroarylene is optionally and independently substituted with 1 or 2 R^{Z}.

In embodiments of the compound of formula (X-II), (II), (II-A), (II-B), (II-C), (II-D), (II-A-1), (II-A-2), (II-A-3), (II-A-4), (II-B3-1), (II-B3-2), (II-B-3), (II-B-4), (II-C-1), (II-C-2), (II-C-3), (II-C-4), (II-D-1), (II-D-2), (II-D-3), or (II-D-4), or a pharmaceutically acceptable salt, stereoisomer, or deuterated form thereof, L¹ comprises —NH(CH₂CH₂O)ₘ—(CH₂)ₙ—, wherein m is an integer of 1-12, and n is an integer of 0-12. In embodiments, m is an integer of 1-12, 1-6, or 1-4. In embodiments, n is an integer of 0-12, 0-8, or 0-3.

In embodiments of the compound of formula (II), (II-A), (II-B), (II-C), (II-D), (II-A-1), (II-A-2), (II-A-3), (II-A-4), (II-B3-1), (II-B3-2), (II-B-3), (II-B-4), (II-C-1), (II-C-2), (II-C-3), (II-C-4), (II-D-1), (II-D-2), (II-D-3), or (II-D-4), or a pharmaceutically acceptable salt, stereoisomer, or deuterated form thereof, L¹ is —(C₁₋₆ alkylene)-NH(CH₂CH₂O)ₘ—(CH₂)ₙ—C CH, —(C₁₋₆ alkylene)-NH(CH₂CH₂O)ₘ—(CH₂)ₙ-arylene-C≡CH, —O—(C₁₋₆ alkylene)-CH(OH)—(C₁₋₆ alkylene)-NH(CH₂CH₂O)ₘ—(CH₂)ₙ—C≡CH, —(C₁₋₆ alkylene)-NH(CH₂CH₂O)ₘ—(CH₂)ₙ—NH—C(O)O—(C₁₋₆ alkyl), —(C₁₋₆ alkylene)-NH(CH₂CH₂O)ₘ—(CH₂)ₙ—C(O)NH—(C₁₋₆ alkyl), —(C₁₋₆ alkylene)-NH(CH₂CH₂O)ₘ—(CH₂)ₙ—C(O)NH—(CH₂)ₙ—C(O)O—(C₁₋₆ alkyl), —(C₁₋₆ alkylene)-NH(CH₂CH₂O)ₘ—(CH₂)ₙ—N(C₁₋₆ alkyl)-C(O)O—(C₁₋₆ alkyl), —(C₁₋₆ alkylene)-NH(CH₂CH₂O)ₘ—(CH₂)ₙ-arylene-C(O)O—(C₁₋₆ alkyl), —(C₁₋₆ alkylene)-NH(CH₂CH₂O)ₘ—(CH₂)ₙ-arylene-NH—C(O)O—(C₁₋₆ alkyl), —(C₁₋₆ alkylene)-NH(CH₂CH₂O)ₘ—(CH₂)ₙ—(C₃₋₈ cycloalkylene)-NH—C(O)O—(C₁₋₆ alkyl), —(C₁₋₆ alkylene)-NH(CH₂CH₂O)ₘ—(CH₂)ₙ—(C₃₋₈ cycloalkylene)-(C₁₋₆ alkylene)-NH—C(O)O—(C₁₋₆ alkyl), —(C₁₋₆ alkylene)-NH(CH₂CH₂O)ₘ—(CH₂)ₙ-heteroarylene-C(O)O—(C₁₋₆ alkyl), —(C₁₋₆ alkylene)-NH(CH₂CH₂O)ₘ—(CH₂)—HET^{A}, —(C₁₋₆ alkylene)-NH(CH₂CH₂O)ₘ—(CH₂)ₙ—C(O)—HET^{A}, —(C₁₋₆ alkylene)-NH(CH₂CH₂O)ₘ—(CH₂)ₙ—NH—(C₁₋₆ alkyl), —(C₁₋₆ alkylene)-NH(CH₂CH₂O)ₘ—(CH₂)ₙ—C(O)O—(C₁₋₆ alkyl), —O—(C₁₋₆ alkylene)-CH(OH)—(C₁₋₆ alkylene)-NH(CH₂CH₂O)ₘ—(CH₂)ₙ—C(O)O—(C₁₋₆ alkyl), —(C₁₋₆ alkylene)-NH(CH₂CH₂O)ₘ—(CH₂)ₙ—O—C₆₋₁₀ aryl, —(C₁₋₆ alkylene)-NH(CH₂CH₂O)ₘ—(CH₂)ₙ—X^{H}, —O—CH₂CH(OH)—(C₁₋₆ alkylene)-NH(CH₂CH₂O)ₘ—(CH₂)ₙ—C≡CH, or —(C₁₋₆ alkylene)-NH(CH₂CH₂O)ₘ—(CH₂)ₙ—(CH₂CH₂O)ₘ—(CH₂)ₙ—C(O)O—(C₁₋₆ alkyl), wherein HET^{A} is a 4-10 membered heterocycle containing 1-3 heteroatoms selected from N or O, and is optionally substituted with —C(O)—(C₁₋₆ alkyl), —C(O)O—(C₁₋₆ alkyl), —(C₁₋₆ alkyl)-O—C₆₋₁₀ aryl, C₁₋₆ alkyl, —(C₁₋₆ alkylene)-C≡CH, —NH—C(O)O—(C₁₋₆ alkyl), —(C₁₋₆ alkylene)-NH—C(O)O—(C₁₋₆ alkyl), —C₁₋₆ alkoxy, —(C₁₋₆ alkylene)-C₁₋₆ alkoxy, or -arylene-C(O)O—(C₁₋₆ alkyl); X^{H} is halogen; each m is independently an integer of 1-12; and each n is independently an integer of 0-12. In embodiments, HET^{A} is azetidinyl, pyrrolidinyl, piperidinyl, piperazinyl, or oxetanyl optionally substituted with —C(O)—(C₁₋₆ alkyl), —C(O)O—(C₁₋₆ alkyl), or —(C₁₋₆ alkyl)-O—C₆₋₁₀ aryl, —(C₁₋₆ alkylene)-C≡CH, —NH—C(O)O—(C₁₋₆ alkyl), —(C₁₋₆ alkylene)-NH—C(O)O—(C₁₋₆ alkyl), —O—C₁₋₆ alkyl, or —(C₁₋₆ alkylene)-O—(C₁₋₆ alkyl). In embodiments, m is an integer of 1-12, 1-6, or 1-4. In embodiments, n is an integer of 0-12, 0-8, or 0-3.

In embodiments of the compound of formula (II), (II-A), (II-B), (II-C), (II-D), (II-A-1), (II-A-2), (II-A-3), (II-A-4), (II-B3-1), (II-B3-2), (II-B-3), (II-B-4), (II-C-1), (II-C-2), (II-C-3), (II-C-4), (II-D-1), (II-D-2), (II-D-3), or (II-D-4), or a pharmaceutically acceptable salt, stereoisomer, or deuterated form thereof, L¹ is —(C₁₋₆ alkylene)-NH(CH₂CH₂O)ₘ—(CH₂)ₙ—C≡CH. In some embodiments, L¹ is —(C₁₋₃ alkylene)-NH—(CH₂CH₂O)ₘ—(CH₂)ₙ—C≡CH. In some embodiments, the alkylene is optionally substituted with 1 or 2 R^{Z}, wherein each R^{Z} is independently —OH, C₁₋₆ alkyl, or halogen. In some embodiments, m is an integer of 1-6. In some embodiments, m is an integer of 1-4. In some embodiments, n is an integer of 0-12. In some embodiments, n is an integer of 0-8. In some embodiments, L¹ is —CH₂NH—(CH₂CH₂O)—(CH₂)₂—C≡CH, —CH₂NH—(CH₂CH₂O)—(CH₂)₅—C≡CH, —CH₂NH—(CH₂CH₂O)—(CH₂)₆—C≡CH, —CH₂NH—(CH₂CH₂O)—(CH₂)₈—C≡CH, —CH₂NH—(CH₂CH₂O)₂—(CH₂)—C≡CH, —CH₂NH—(CH₂CH₂O)₂—(CH₂)₂—C≡CH, —CH₂NH—(CH₂CH₂O)₃—(CH₂)—C≡CH, or —CH₂NH—(CH₂CH₂O)₃—(CH₂)₂—C≡CH.

In embodiments of the compound of formula (II), (II-A), (II-B), (II-C), (II-D), (II-A-1), (II-A-2), (II-A-3), (II-A-4), (II-B3-1), (II-B3-2), (II-B-3), (II-B-4), (II-C-1), (II-C-2), (II-C-3), (II-C-4), (II-D-1), (II-D-2), (II-D-3), or (II-D-4), or a pharmaceutically acceptable salt, stereoisomer, or deuterated form thereof, L¹ is —(C₁₋₆ alkylene)-NH(CH₂CH₂O)ₘ—(CH₂)ₙ—C≡CH. In some embodiments, L¹ is —(C₁₋₃ alkylene)-NH—(CH₂CH₂O)ₘ—(CH₂)ₙ—C≡CH. In some embodiments, the alkylene is optionally substituted with 1 or 2 R^{Z}, wherein each R^{Z} is independently —OH, C₁₋₆ alkyl, or halogen. In some embodiments, m is an integer of 1-6. In some embodiments, m is an integer of 1-4. In some embodiments, n is an integer of 0-3. In some embodiments, L¹ is —CH₂NH—(CH₂CH₂O)₂—(CH₂)₂—C≡CH or —CH₂NH—(CH₂CH₂O)₃—(CH₂)₂—C≡CH.

In embodiments of the compound of formula (II), (II-A), (II-B), (II-C), (II-D), (II-A-1), (II-A-2), (II-A-3), (II-A-4), (II-B3-1), (II-B3-2), (II-B-3), (II-B-4), (II-C-1), (II-C-2), (II-C-3), (II-C-4), (II-D-1), (II-D-2), (II-D-3), or (II-D-4), or a pharmaceutically acceptable salt, stereoisomer, or deuterated form thereof, L¹ is —(C₁₋₆ alkylene)-NH(CH₂CH₂O)ₘ—(CH₂)ₙ-arylene-C≡CH. In some embodiments, L¹ is —(C₁₋₃ alkylene)-NH(CH₂CH₂O)ₘ—(CH₂)ₙ-arylene-C≡CH. In some embodiments, the arylene is optionally substituted phenylene. In some embodiments, the alkylene is optionally substituted with 1 or 2 R^{Z}, wherein each R^{Z} is independently —OH, C₁₋₆ alkyl, or halogen. In some embodiments, m is an integer of 1-6. In some embodiments, m is an integer of 1-4. In some embodiments, n is an integer of 0-3. In some embodiments, L is

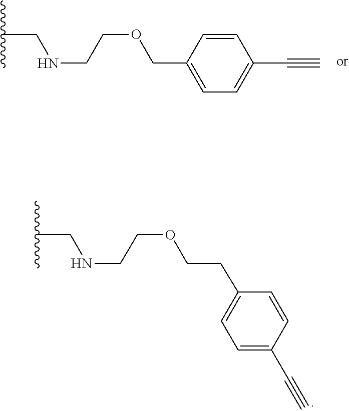 [SMILES: *CNCCOCc1ccc(C#C)cc1 ; *CNCCOCCc1ccc(C#C)cc1]

In embodiments of the compound of formula (II), (II-A), (II-B), (II-C), (II-D), (II-A-1), (II-A-2), (II-A-3), (II-A-4), (II-B3-1), (II-B3-2), (II-B-3), (II-B-4), (II-C-1), (II-C-2), (II-C-3), (II-C-4), (II-D-1), (II-D-2), (II-D-3), or (II-D-4), or a pharmaceutically acceptable salt, stereoisomer, or deuterated form thereof, L¹ is —O—(C₁₋₆ alkylene)-CH(OH)—(C₁₋₆ alkylene)-NH(CH₂CH₂O)ₘ—(CH₂)ₙ—C≡CH. In some embodiments, L¹ is —O—(C₁₋₃ alkylene)-CH(OH)—(C₁₋₃ alkylene)-NH(CH₂CH₂O)ₘ—(CH₂)ₙ—C≡CH. In some embodiments, the alkylene is optionally substituted with 1 or 2 R^{Z}, wherein each R^{Z} is independently —OH, C₁₋₆ alkyl, or halogen. In some embodiments, m is an integer of 1-6. In some embodiments, m is an integer of 1-4. In some embodiments, n is an integer of 0-4. In some embodiments, n is an integer of 1-3. In some embodiments, L¹ is —O—CH₂—CH(OH)—CH₂—NH—(CH₂CH₂O)₂—(CH₂)₂—C≡CH.

In embodiments of the compound of formula (II), (II-A), (II-B), (II-C), (II-D), (II-A-1), (II-A-2), (II-A-3), (II-A-4), (II-B3-1), (II-B3-2), (II-B-3), (II-B-4), (II-C-1), (II-C-2), (II-C-3), (II-C-4), (II-D-1), (II-D-2), (II-D-3), or (II-D-4), or a pharmaceutically acceptable salt, stereoisomer, or deuterated form thereof, L¹ is —(C₁₋₆ alkylene)-NH(CH₂CH₂O)ₘ—(CH₂)ₙ—NH—C(O)O—(C₁₋₆ alkyl). In some embodiments, the alkylene or alkyl is optionally and independently substituted with 1, 2, or 3 R^{Z}, wherein each R^{Z} is independently —OH, C₁₋₆ alkyl, or halogen. In some embodiments, m is an integer of 1-6. In some embodiments, m is an integer of 1-4. In some embodiments, n is an integer of 0-12. In some embodiments, n is an integer of 0-8. In some embodiments, L¹ is —CH₂NH—(CH₂CH₂O)—(CH₂)₂—NHC(O)OC(CH₃)₃, —CH₂NH—(CH₂CH₂O)—(CH₂)₃—NHC(O)OC(CH₃)₃, —CH₂NH—(CH₂CH₂O)—(CH₂)₅—NHC(O)OC(CH₃)₃, —CH₂NH—(CH₂CH₂O)—(CH₂)₆—NHC(O)OC(CH₃)₃, —CH₂NH—(CH₂CH₂O)—(CH₂)₈—NHC(O)OC(CH₃)₃, —CH₂NH—(CH₂CH₂O)₂—(CH₂)₂—NHC(O)OC(CH₃)₃, or —CH₂NH—(CH₂CH₂O)₃—(CH₂)₂—NHC(O)OC(CH₃)₃.

In embodiments of the compound of formula (II), (II-A), (II-B), (II-C), (II-D), (II-A-1), (II-A-2), (II-A-3), (II-A-4), (II-B3-1), (II-B3-2), (II-B-3), (II-B-4), (II-C-1), (II-C-2), (II-C-3), (II-C-4), (II-D-1), (II-D-2), (II-D-3), or (II-D-4), or a pharmaceutically acceptable salt, stereoisomer, or deuterated form thereof, L¹ is —(C₁₋₆ alkylene)-NH(CH₂CH₂O)ₘ—(CH₂)ₙ—NH—C(O)O—(C₁₋₆ alkyl). In some embodiments, the alkylene or alkyl is optionally and independently substituted with 1, 2, or 3 R^{Z}, wherein each R^{Z} is independently —OH, C₁₋₆ alkyl, or halogen. In some embodiments, L¹ is —CH₂NH—[CH₂CH₂O]₃—[CH₂]₂—NHC(O)OC(CH₃)₃.

In embodiments of the compound of formula (II), (II-A), (II-B), (II-C), (II-D), (II-A-1), (II-A-2), (II-A-3), (II-A-4), (II-B3-1), (II-B3-2), (II-B-3), (II-B-4), (II-C-1), (II-C-2), (II-C-3), (II-C-4), (II-D-1), (II-D-2), (II-D-3), or (II-D-4), or a pharmaceutically acceptable salt, stereoisomer, or deuterated form thereof, L¹ is —(C₁₋₆ alkylene)-NH(CH₂CH₂O)ₘ—(CH₂)ₙ—(C₃₋₈ cycloalkylene)-NH—C(O)O—(C₁₋₆ alkyl) or —(C₁₋₆ alkylene)-NH(CH₂CH₂O)ₘ—(CH₂)ₙ—(C₃₋₈ cycloalkylene)-(C₁₋₆ alkylene)-NH—C(O)O—(C₁₋₆ alkyl). In some embodiments, the alkylene, cycloalkylene, or alkyl is optionally and independently substituted with 1, 2, or 3 R^{Z}, wherein each R^{Z} is independently —OH, C₁₋₆ alkyl, or halogen. In some embodiments, the C₃₋₈ cycloalkylene is

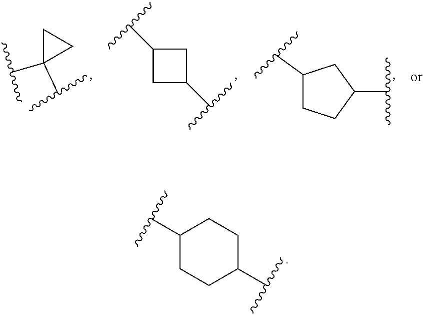 [SMILES: *C1(*)CC1 ; *C1CC(*)C1 ; *C1CCC(*)C1 ; *C1CCC(*)CC1]

In some embodiments, m is an integer of 1-6. In some embodiments, m is an integer of 1-4. In some embodiments, n is an integer of 0-12. In some embodiments, n is an integer of 0-8. In some embodiments, L¹ is

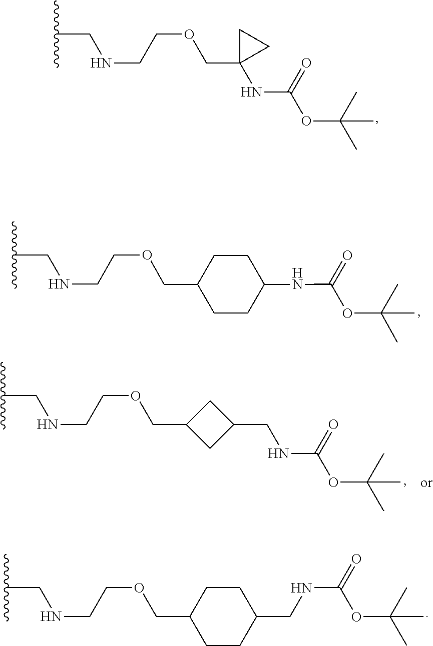 [SMILES: *CNCCOCC1(NC(=O)OC(C)(C)C)CC1 ; *CNCCOCC1CCC(NC(=O)OC(C)(C)C)CC1 ; *CNCCOCC1CC(CNC(=O)OC(C)(C)C)C1 ; *CNCCOCC1CCC(CNC(=O)OC(C)(C)C)CC1]

In embodiments of the compound of formula (II), (II-A), (II-B), (II-C), (II-D), (II-A-1), (II-A-2), (II-A-3), (II-A-4), (II-B3-1), (II-B3-2), (II-B-3), (II-B-4), (II-C-1), (II-C-2), (II-C-3), (II-C-4), (II-D-1), (II-D-2), (II-D-3), or (II-D-4), or a pharmaceutically acceptable salt, stereoisomer, or deuterated form thereof, L¹ is —(C₁₋₆ alkylene)-NH(CH₂CH₂O)ₘ—(CH₂)ₙ-arylene-NH—C(O)O—(C₁₋₆ alkyl). In some embodiments, the alkylene, arylene, or alkyl is optionally and independently substituted with 1, 2, or 3 R^{Z}, wherein each R^{Z} is independently —OH, C₁₋₆ alkyl, or halogen. In some embodiments, the arylene is phenylene. In embodiments, the phenylene is p-phenylene, m-phenylene o-phenylene. In some embodiments, m is an integer of 1-6. In some embodiments, m is an integer of 1-4. In some embodiments, n is an integer of 0-12. In some embodiments, n is an integer of 0-6. In some embodiments, L¹ is

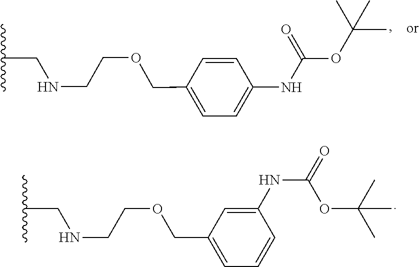 [SMILES: *CNCCOCc1ccc(NC(=O)OC(C)(C)C)cc1 ; *CNCCOCc1cccc(NC(=O)OC(C)(C)C)c1]

In embodiments of the compound of formula (II), (II-A), (II-B), (II-C), (II-D), (II-A-1), (II-A-2), (II-A-3), (II-A-4), (II-B3-1), (II-B3-2), (II-B-3), (II-B-4), (II-C-1), (II-C-2), (II-C-3), (II-C-4), (II-D-1), (II-D-2), (II-D-3), or (II-D-4), or a pharmaceutically acceptable salt, stereoisomer, or deuterated form thereof, L¹ is —(C₁₋₆ alkylene)-NH(CH₂CH₂O)ₘ—(CH₂)ₙ—N(C₁. 6 alkyl)-C(O)O—(C₁₋₆ alkyl). In some embodiments, the alkylene or alkyl is optionally and independently substituted with 1, 2, or 3 R^{Z}, wherein each R^{Z} is independently —OH, C₁₋₆ alkyl, or halogen. In some embodiments, L¹ is —CH₂NH—(CH₂CH₂O)₃—(CH₂)₃—NCH₃C(O)OC(CH₃)₃, or —CH₂NH—(CH₂CH₂O)₄—(CH₂)₃—NCH₃C(O)OC(CH₃)₃.

In embodiments of the compound of formula (II), (II-A), (II-B), (II-C), (II-D), (II-A-1), (II-A-2), (II-A-3), (II-A-4), (II-B3-1), (II-B3-2), (II-B-3), (II-B-4), (II-C-1), (II-C-2), (II-C-3), (II-C-4), (II-D-1), (II-D-2), (II-D-3), or (II-D-4), or a pharmaceutically acceptable salt, stereoisomer, or deuterated form thereof, L¹ is —(C₁₋₆ alkylene)-NH(CH₂CH₂O)ₘ—(CH₂)—HET^{A}, or —(C₁₋₆ alkylene)-NH(CH₂CH₂O)ₘ—(CH₂)ₙ—C(O)—HET^{A}, wherein HET^{A} is a 4-10 membered heterocycle containing 1-3 heteroatoms selected from N or O, and is optionally substituted with —C(O)—(C₁₋₆ alkyl), —C(O)O—(C₁₋₆ alkyl), —(C₁₋₆ alkyl)-O—C₆₋₁₀ aryl, C₁₋₆ alkyl, —(C₁₋₆ alkylene)-C≡CH, —NH—C(O)O—(C₁₋₆ alkyl), —(C₁₋₆ alkylene)-NH—C(O)O—(C₁₋₆ alkyl), —C₁₋₆ alkoxy, —(C₁₋₆ alkylene)-C₁₋₆ alkoxy, or -arylene-C(O)O—(C₁₋₆ alkyl). In some embodiments, the alkylene is optionally substituted with 1, 2, or 3 R^{Z}, wherein each R^{Z} is independently —OH, C₁₋₆ alkyl, or halogen. In some embodiments, m is an integer of 1-6. In some embodiments, m is an integer of 1-4. In some embodiments, n is an integer of 0-12. In some embodiments, n is an integer of 0-6. In some embodiments, HET^{A} is azetidinyl, pyrrolidinyl, piperidinyl, piperazinyl, or oxetanyl optionally substituted with —C(O)—(C₁₋₆ alkyl), —C(O)O—(C₁₋₆ alkyl), —(C₁₋₆ alkyl)-O—C₆₋₁₀ aryl, —(C₁₋₆ alkylene)-C≡CH, —NH—C(O)O—(C₁₋₆ alkyl), —(C₁₋₆ alkylene)-NH—C(O)O—(C₁₋₆ alkyl), —O—C₁₋₆ alkyl, or —(C₁₋₆ alkylene)-O—(C₁₋₆ alkyl). In some embodiments, HET^{A} is azetidinyl, pyrrolidinyl, piperidinyl, piperazinyl, or oxetanyl optionally substituted with —C(O)—(C₁₋₆ alkyl), —C(O)O—(C₁₋₆ alkyl), or —(C₁₋₆ alkyl)-O—C₆₋₁₀ aryl. In some embodiments, HET^{A} is

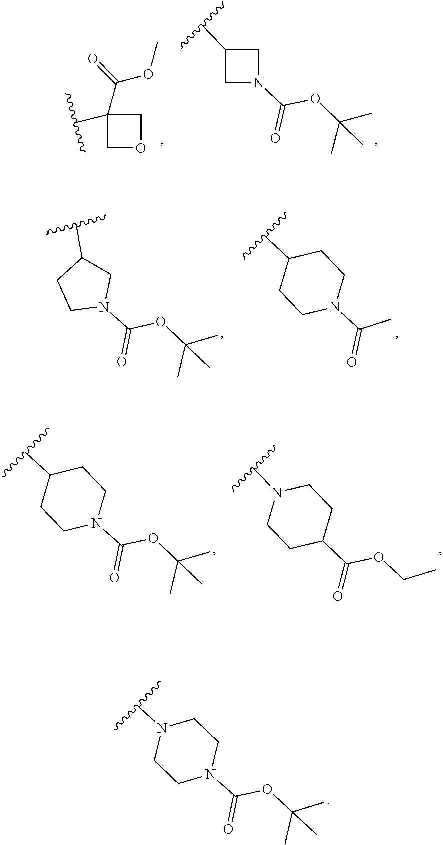

In some embodiments L¹ is

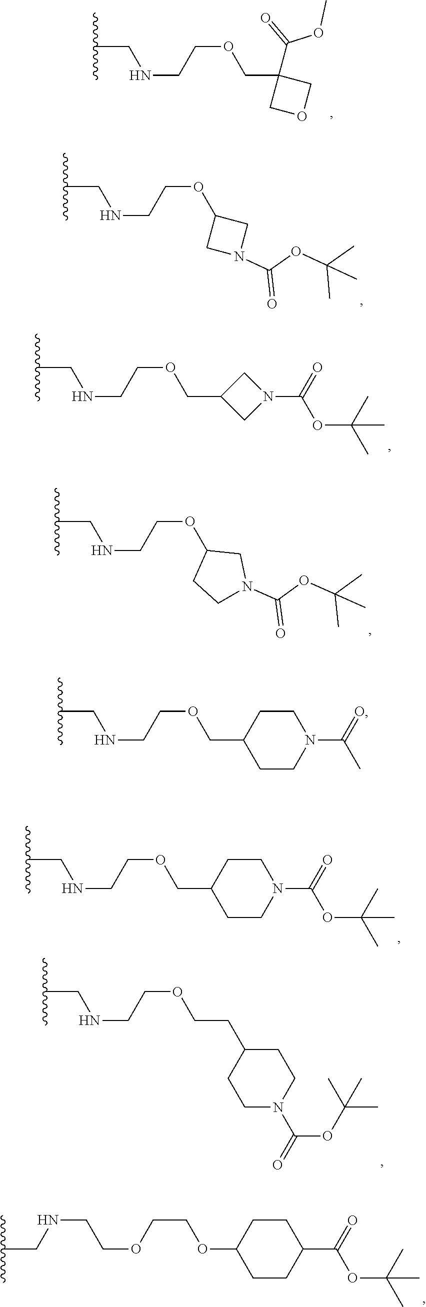

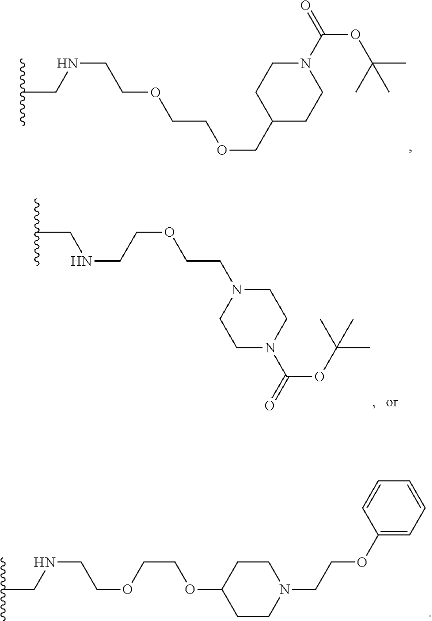 [SMILES: *CNCCOCCOCC1CCN(C(=O)OC(C)(C)C)CC1 ; *CNCCOCCN1CCN(C(=O)OC(C)(C)C)CC1 ; *CNCCOCCOC1CCN(CCOc2ccccc2)CC1]

In some embodiments, L¹ is

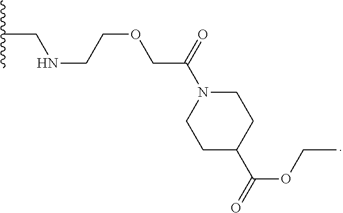 [SMILES: *CNCCOCC(=O)N1CCC(C(=O)OCC)CC1]

In some embodiments, HET^{A} is azetidinyl, pyrrolidinyl, piperidinyl, piperazinyl, or oxetanyl optionally substituted with —O—C₁₋₆ alkyl or —(C₁₋₆ alkylene)-O—(C₁₋₆ alkyl). In some embodiments, HET^{A} is

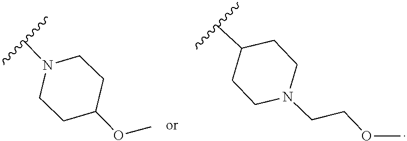

In some embodiments, L¹ is

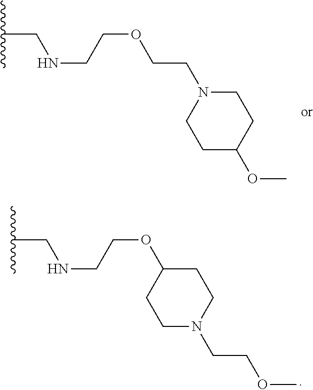 [SMILES: *CNCCOCCN1CCC(OC)CC1 ; *CNCCOC1CCN(CCOC)CC1]

In some embodiments, HET^{A} is azetidinyl, pyrrolidinyl, piperidinyl, piperazinyl, or oxetanyl optionally substituted with —(C₁₋₆ alkylene)-C≡CH. In some embodiments, HET^{A} is

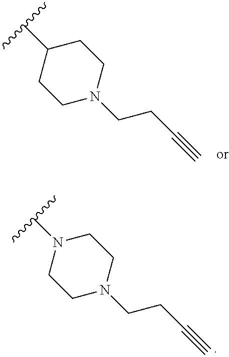 [SMILES: *C1CCN(CCC#C)CC1 ; *N1CCN(CCC#C)CC1]

In some embodiments, L¹ is

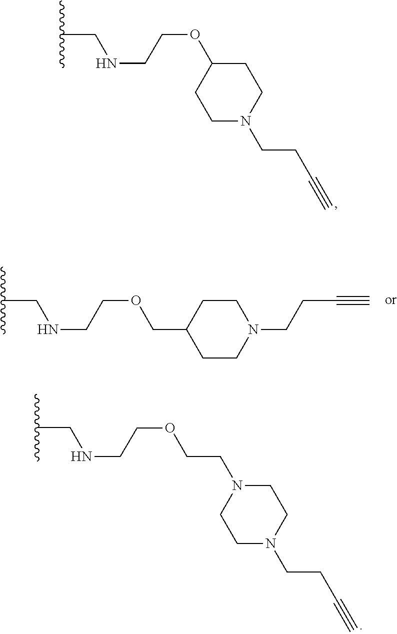 [SMILES: *CNCCOC1CCN(CCC#C)CC1 ; *CNCCOCC1CCN(CCC#C)CC1 ; *CNCCOCCN1CCN(CCC#C)CC1]

In some embodiments, HET^{A} is azetidinyl, pyrrolidinyl, piperidinyl, piperazinyl, or oxetanyl optionally substituted with —NH—C(O)O—(C₁₋₆ alkyl) or —(C₁₋₆ alkylene)-NH—C(O)O—(C₁₋₆ alkyl). In some embodiments, HET^{A} is

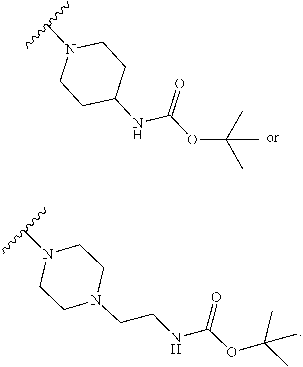 [SMILES: *N1CCC(NC(=O)OC(C)(C)C)CC1 ; *N1CCN(CCNC(=O)OC(C)(C)C)CC1]

In some embodiments, L¹ is

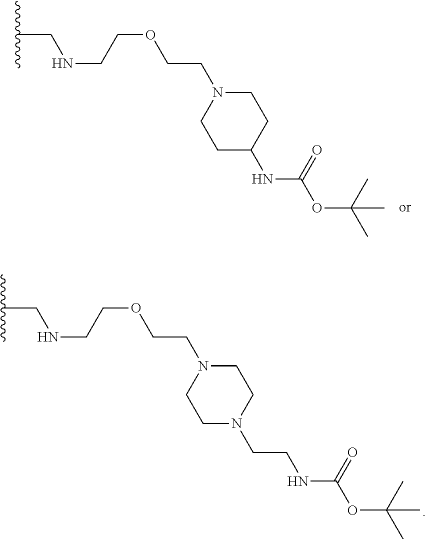 [SMILES: *CNCCOCCN1CCC(NC(=O)OC(C)(C)C)CC1 ; *CNCCOCCN1CCN(CCNC(=O)OC(C)(C)C)CC1]

In some embodiments, HET^{A} is azetidinyl, pyrrolidinyl, piperidinyl, piperazinyl, or oxetanyl optionally substituted with -arylene-C(O)O—(C₁₋₆ alkyl). In some embodiments, the arylene is optionally substituted phenylene. In some embodiments, HET^{A} is

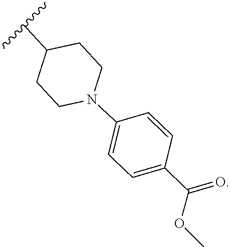 [SMILES: *C1CCN(c2ccc(C(=O)OC)cc2)CC1]

In some embodiments, L¹ is

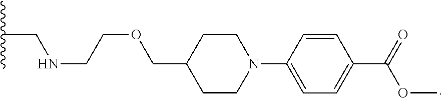 [SMILES: *CNCCOCC1CCN(c2ccc(C(=O)OC)cc2)CC1]

In some embodiments, HET^{A} is piperidinyl optionally substituted with —C(O)—(C₁₋₆ alkyl), —C(O)O—(C₁₋₆ alkyl), or —(C₁₋₆ alkyl)-O—C₆₋₁₀ aryl. In some embodiments, HET^{A} is

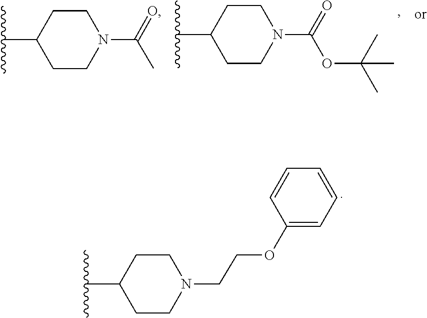

In some embodiments, L¹ is

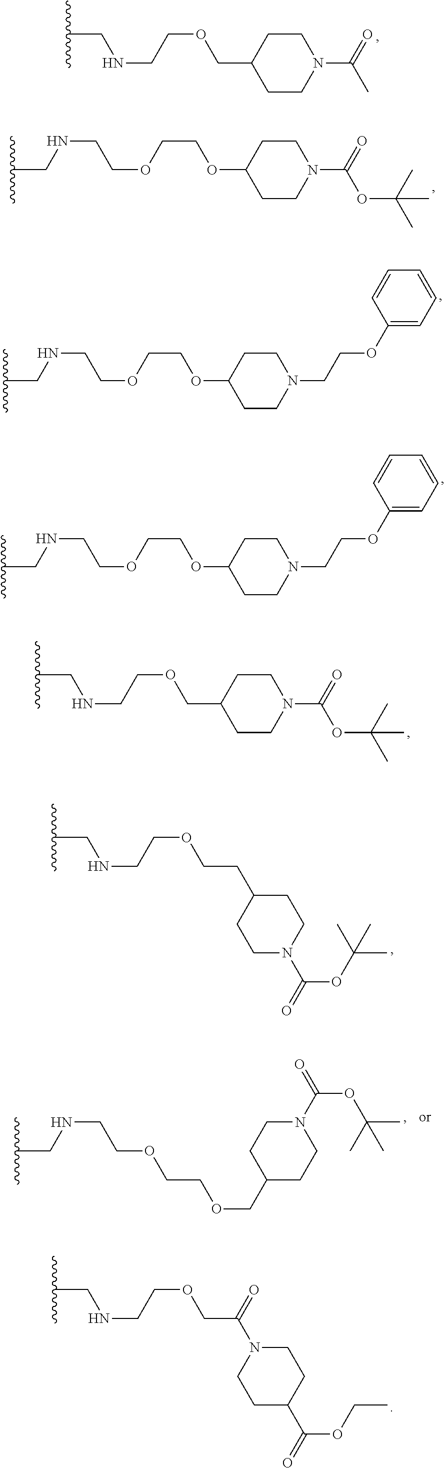

In embodiments of the compound of formula (II), (II-A), (II-B), (II-C), (II-D), (II-A-1), (II-A-2), (II-A-3), (II-A-4), (II-B3-1), (II-B3-2), (II-B-3), (II-B-4), (II-C-1), (II-C-2), (II-C-3), (II-C-4), (II-D-1), (II-D-2), (II-D-3), or (II-D-4), or a pharmaceutically acceptable salt, stereoisomer, or deuterated form thereof, L¹ is —(C₁₋₆ alkylene)-NH(CH₂CH₂O)ₘ—(CH₂)—HET^{A}, wherein HET^{A} is a 4-10 membered heterocycle containing 1-3 N, and is optionally substituted with —C(O)—(C₁₋₆ alkyl), —C(O)O—(C₁₋₆ alkyl), —(C₁₋₆ alkyl)-O—C₆₋₁₀ aryl, or C₁₋₆ alkyl. In some embodiments, the alkylene is optionally substituted with 1, 2, or 3 R^{Z}, wherein each R^{Z} is independently —OH, C₁₋₆ alkyl, or halogen. In some embodiments, HET^{A} is piperidinyl optionally substituted with —C(O)—(C₁₋₆ alkyl), —C(O)O—(C₁₋₆ alkyl), or —(C₁₋₆ alkyl)-O—C₆₋₁₀ aryl. In some embodiments, HET^{A} is

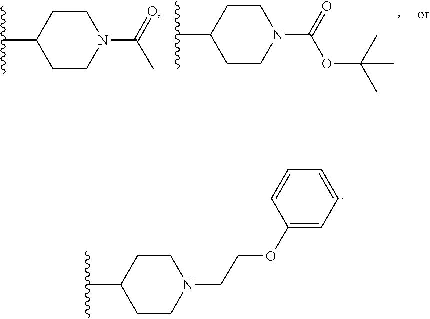

In some embodiments, L¹ is

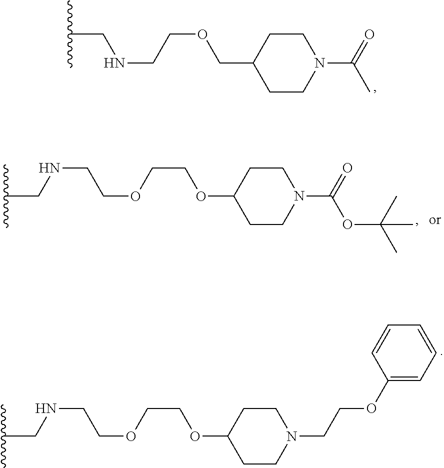 [SMILES: *CNCCOCC1CCN(C(C)=O)CC1 ; *CNCCOCCOC1CCN(C(=O)OC(C)(C)C)CC1 ; *CNCCOCCOC1CCN(CCOc2ccccc2)CC1]

In embodiments of the compound of formula (II), (II-A), (II-B), (II-C), (II-D), (II-A-1), (II-A-2), (II-A-3), (II-A-4), (II-B3-1), (II-B3-2), (II-B-3), (II-B-4), (II-C-1), (II-C-2), (II-C-3), (II-C-4), (II-D-1), (II-D-2), (II-D-3), or (II-D-4), or a pharmaceutically acceptable salt, stereoisomer, or deuterated form thereof, L¹ is —(C₁₋₆ alkylene)-NH(CH₂CH₂O)ₘ—(CH₂)ₙ—NH—(C₁₋₆ alkyl). In some embodiments, the alkylene or alkyl is optionally and independently substituted with 1, 2, or 3 R^{Z}, wherein each R^{Z} is independently —OH, C₁₋₆ alkyl, or halogen. In some embodiments, m is an integer of 1-6. In some embodiments, m is an integer of 1-4. In some embodiments, n is an integer of 0-12. In some embodiments, n is an integer of 0-6. In some embodiments, L¹ is —(C₁₋₆ alkylene)-NH(CH₂CH₂O)ₘ—(CH₂)ₙ—NHCH₃. In some embodiments, L¹ is —CH₂NH—[CH₂CH₂O]₂—[CH₂]₂—NHCH₃, —CH₂NH—(CH₂CH₂O)₂—(CH₂)₃—NHCH₃, or —CH₂NH—[CH₂CH₂O]₃—[CH₂]₂—NHCH₃.

In embodiments of the compound of formula (II), (II-A), (II-B), (II-C), (II-D), (II-A-1), (II-A-2), (II-A-3), (II-A-4), (II-B3-1), (II-B3-2), (II-B-3), (II-B-4), (II-C-1), (II-C-2), (II-C-3), (II-C-4), (II-D-1), (II-D-2), (II-D-3), or (II-D-4), or a pharmaceutically acceptable salt, stereoisomer, or deuterated form thereof, L¹ is —(C₁₋₆ alkylene)-NH(CH₂CH₂O)ₘ—(CH₂)ₙ—NH—(C₁₋₆ alkyl). In some embodiments, the alkylene or alkyl is optionally and independently substituted with 1, 2, or 3 R^{Z}, wherein each R^{Z} is independently —OH, C₁₋₆ alkyl, or halogen. In some embodiments, L¹ is —(C₁₋₆ alkylene)-NH(CH₂CH₂O)ₘ—(CH₂)ₙ—NHCH₃. In some embodiments, L¹ is —CH₂NH—[CH₂CH₂O]₂—[CH₂]₂—NHCH₃, or —CH₂NH—[CH₂CH₂O]₃—[CH₂]₂—NHCH₃.

In embodiments of the compound of formula (II), (II-A), (II-B), (II-C), (II-D), (II-A-1), (II-A-2), (II-A-3), (II-A-4), (II-B3-1), (II-B3-2), (II-B-3), (II-B-4), (II-C-1), (II-C-2), (II-C-3), (II-C-4), (II-D-1), (II-D-2), (II-D-3), or (II-D-4), or a pharmaceutically acceptable salt, stereoisomer, or deuterated form thereof, L¹ is —(C₁₋₆ alkylene)-NH(CH₂CH₂O)ₘ—(CH₂)ₙ—C(O)NH—(C₁₋₆ alkyl). In some embodiments, the alkylene or alkyl is optionally and independently substituted with 1, 2, or 3 R^{Z}, wherein each R^{Z} is independently —OH, C₁₋₆ alkyl, or halogen. In some embodiments, m is an integer of 1-6. In some embodiments, m is an integer of 1-4. In some embodiments, n is an integer of 0-12. In some embodiments, n is an integer of 0-6. In embodiments, L¹ is —(C₁₋₃ alkylene)-NH(CH₂CH₂O)ₘ—(CH₂)ₙ—C(O)NH—CH₃. In some embodiments, L¹ is —CH₂NH—(CH₂CH₂O)₂—(CH₂)₂—C(O)NH—CH₃.

In embodiments of the compound of formula (II), (II-A), (II-B), (II-C), (II-D), (II-A-1), (II-A-2), (II-A-3), (II-A-4), (II-B3-1), (II-B3-2), (II-B-3), (II-B-4), (II-C-1), (II-C-2), (II-C-3), (II-C-4), (II-D-1), (II-D-2), (II-D-3), or (II-D-4), or a pharmaceutically acceptable salt, stereoisomer, or deuterated form thereof, L¹ is —(C₁₋₆ alkylene)-NH(CH₂CH₂O)ₘ—(CH₂)ₙ—C(O)NH—(CH₂)ₙ—C(O)O—(C₁₋₆ alkyl). In some embodiments, the alkylene or alkyl is optionally and independently substituted with 1, 2, or 3 R^{Z}, wherein each R^{Z} is independently —OH, C₁₋₆ alkyl, or halogen. In some embodiments, m is an integer of 1-6. In some embodiments, m is an integer of 1-4. In some embodiments, n is an integer of 0-12. In embodiments, L¹ is —(C₁₋₆ alkylene)-NH(CH₂CH₂O)ₘ—(CH₂)ₙ—C(O)NH—(CH₂)ₙ—C(O)OCH₂CH₃. In embodiments, L¹ is —CH₂NH—(CH₂CH₂O)—(CH₂)—C(O)NH—(CH₂)—C(O)OCH₂CH₃.

In embodiments of the compound of formula (II), (II-A), (II-B), (II-C), (II-D), (II-A-1), (II-A-2), (II-A-3), (II-A-4), (II-B3-1), (II-B3-2), (II-B-3), (II-B-4), (II-C-1), (II-C-2), (II-C-3), (II-C-4), (II-D-1), (II-D-2), (II-D-3), or (II-D-4), or a pharmaceutically acceptable salt, stereoisomer, or deuterated form thereof, L¹ is —(C₁₋₆ alkylene)-NH(CH₂CH₂O)ₘ—(CH₂)ₙ—C(O)O—(C₁₋₆ alkyl). In some embodiments, L¹ is —(C₁₋₆ alkylene)-NH(CH₂CH₂O)ₘ—(CH₂)ₙ—C(O)O—(C₁₋₄ alkyl). In some embodiments, the alkylene or alkyl is optionally and independently substituted with 1, 2, or 3 R^{Z}, wherein each R^{Z} is independently —OH, C₁₋₆ alkyl, or halogen. In some embodiments, L¹ is —(C₁₋₆ alkylene)-NH(CH₂CH₂O)ₘ—(CH₂)ₙ—C(O)OCH₃ or —(C₁₋₆ alkylene)-NH(CH₂CH₂O)ₘ—(CH₂)ₙ—C(O)OC(CH₃)₃. In some embodiments, m is an integer of 1-6. In some embodiments, m is an integer of 1-4. In some embodiments, n is an integer of 0-12. In some embodiments, n is an integer of 0-8. In some embodiments, L¹ is —CH₂NH—[CH₂CH₂O]₂—[CH₂]₂—C(O)OCH₃, —CH₂NH—(CH₂CH₂O)₃—(CH₂)₂—C(O)OCH₃, —CH₂NH(CH₂CH₂O)—(CH₂)—C(O)OC(CH₃)₃, —CH₂NH(CH₂CH₂O)—(CH₂)₂—C(O)OC(CH₃)₃, —CH₂NH(CH₂CH₂O)—(CH₂)₃—C(O)OC(CH₃)₃, —CH₂NH(CH₂CH₂O)—(CH₂)₄—C(O)OC(CH₃)₃, —CH₂NH(CH₂CH₂O)—(CH₂)₅—C(O)OC(CH₃)₃, —CH₂NH(CH₂CH₂O)—(CH₂)₈—C(O)OC(CH₃)₃, or —CH₂NH(CH₂CH₂O)₂—(CH₂)₂—C(O)OC(CH₃)₃.

In embodiments of the compound of formula (II), (II-A), (II-B), (II-C), (II-D), (II-A-1), (II-A-2), (II-A-3), (II-A-4), (II-B3-1), (II-B3-2), (II-B-3), (II-B-4), (II-C-1), (II-C-2), (II-C-3), (II-C-4), (II-D-1), (II-D-2), (II-D-3), or (II-D-4), or a pharmaceutically acceptable salt, stereoisomer, or deuterated form thereof, L¹ is —(C₁₋₆ alkylene)-NH(CH₂CH₂O)ₘ—(CH₂)ₙ—C(O)O—(C₁₋₆ alkyl). In some embodiments, L¹ is —(C₁₋₆ alkylene)-NH(CH₂CH₂O)ₘ—(CH₂)ₙ—C(O)O—(C₁₋₃ alkyl). In some embodiments, the alkylene or alkyl is optionally and independently substituted with 1, 2, or 3 R^{Z}, wherein each R^{Z} is independently —OH, C₁₋₆ alkyl, or halogen. In some embodiments, L¹ is —(C₁₋₆ alkylene)-NH(CH₂CH₂O)ₘ—(CH₂)ₙ—C(O)OCH₃. In some embodiments, L¹ is —CH₂NH—[CH₂CH₂O]₂—[CH₂]₂—C(O)OCH₃.

In embodiments of the compound of formula (II), (II-A), (II-B), (II-C), (II-D), (II-A-1), (II-A-2), (II-A-3), (II-A-4), (II-B3-1), (II-B3-2), (II-B-3), (II-B-4), (II-C-1), (II-C-2), (II-C-3), (II-C-4), (II-D-1), (II-D-2), (II-D-3), or (II-D-4), or a pharmaceutically acceptable salt, stereoisomer, or deuterated form thereof, L¹ is —O—(C₁₋₆ alkylene)-CH(OH)—(C₁₋₆ alkylene)-NH(CH₂CH₂O)ₘ—(CH₂)ₙ—C(O)O—(C₁₋₆ alkyl). In some embodiments, L¹ is —O—(C₁₋₃ alkylene)-CH(OH)—(C₁₋₃ alkylene)-NH(CH₂CH₂O)ₘ—(CH₂)ₙ—C(O)O—(C₁₋₃ alkyl). In some embodiments, the alkylene is optionally substituted with 1 or 2 R^{Z}, wherein each R^{Z} is independently —OH, C₁₋₆ alkyl, or halogen. In some embodiments, m is an integer of 1-6. In some embodiments, m is an integer of 1-4. In some embodiments, n is an integer of 0-4. In some embodiments, n is an integer of 1-3. In some embodiments, L¹ is —O—CH₂—CH(OH)—CH₂NH—(CH₂CH₂O)—(CH₂)₂—C(O)OCH₃.

In embodiments of the compound of formula (II), (II-A), (II-B), (II-C), (II-D), (II-A-1), (II-A-2), (II-A-3), (II-A-4), (II-B3-1), (II-B3-2), (II-B-3), (II-B-4), (II-C-1), (II-C-2), (II-C-3), (II-C-4), (II-D-1), (II-D-2), (II-D-3), or (II-D-4), or a pharmaceutically acceptable salt, stereoisomer, or deuterated form thereof, L¹ is —(C₁₋₆ alkylene)-NH(CH₂CH₂O)ₘ—(CH₂)ₙ—O—C₆₋₁₀ aryl. In some embodiments, m is an integer of 1-6. In some embodiments, m is an integer of 1-4. In some embodiments, n is an integer of 0-12. In some embodiments, n is an integer of 0-6. In some embodiments, the alkylene or aryl is optionally and independently substituted with 1, 2, or 3 R^{Z}, wherein each R^{Z} is independently —OH, C₁₋₆ alkyl, or halogen. In some embodiments, L¹ is

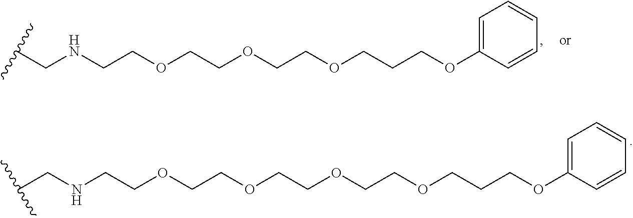 [SMILES: *CNCCOCCOCCOCCCOc1ccccc1 ; *CNCCOCCOCCOCCOCCCOc1ccccc1]

In embodiments of the compound of formula (II), (II-A), (II-B), (II-C), (II-D), (II-A-1), (II-A-2), (II-A-3), (II-A-4), (II-B3-1), (II-B3-2), (II-B-3), (II-B-4), (II-C-1), (II-C-2), (II-C-3), (II-C-4), (II-D-1), (II-D-2), (II-D-3), or (II-D-4), or a pharmaceutically acceptable salt, stereoisomer, or deuterated form thereof, L¹ is —(C₁₋₆ alkylene)-NH(CH₂CH₂O)ₘ—(CH₂)ₙ-arylene-C(O)O—(C₁₋₆ alkyl). In some embodiments, the alkylene, arylene or alkyl is optionally and independently substituted with 1, 2, or 3 R^{Z}, wherein each R^{Z} is independently —OH, C₁₋₆ alkyl, or halogen. In some embodiments, the arylene is phenylene. In embodiments, the phenylene is p-phenylene, m-phenylene o-phenylene. In some embodiments, m is an integer of 1-6. In some embodiments, m is an integer of 1-4. In some embodiments, n is an integer of 0-12. In some embodiments, n is an integer of 0-6. In some embodiments, L¹ is

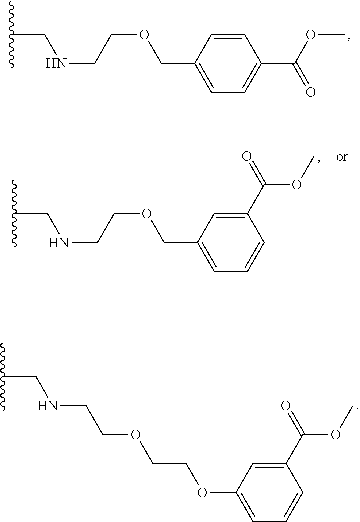 [SMILES: *CNCCOCc1ccc(C(=O)OC)cc1 ; *CNCCOCc1cccc(C(=O)OC)c1 ; *CNCCOCCOc1cccc(C(=O)OC)c1]

In embodiments of the compound of formula (II), (II-A), (II-B), (II-C), (II-D), (II-A-1), (II-A-2), (II-A-3), (II-A-4), (II-B3-1), (II-B3-2), (II-B-3), (II-B-4), (II-C-1), (II-C-2), (II-C-3), (II-C-4), (II-D-1), (II-D-2), (II-D-3), or (II-D-4), or a pharmaceutically acceptable salt, stereoisomer, or deuterated form thereof, L¹ is —(C₁₋₆ alkylene)-NH(CH₂CH₂O)ₘ—(CH₂)ₙ-heteroarylene-C(O)O—(C₁₋₆ alkyl). In some embodiments, the alkylene, heteroarylene or alkyl is optionally and independently substituted with 1, 2, or 3 R^{Z}, wherein each R^{Z} is independently —OH, C₁₋₆ alkyl, or halogen. In some embodiments, the heteroarylene is pyridinylene. In some embodiments, m is an integer of 1-6. In some embodiments, m is an integer of 1-4. In some embodiments, n is an integer of 0-12. In some embodiments, n is an integer of 0-6. In some embodiments, L¹ is

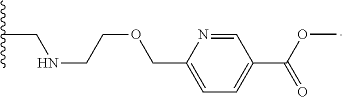 [SMILES: *CNCCOCc1ccc(C(=O)OC)cn1]

In embodiments of the compound of formula (II), (II-A), (II-B), (II-C), (II-D), (II-A-1), (II-A-2), (II-A-3), (II-A-4), (II-B3-1), (II-B3-2), (II-B-3), (II-B-4), (II-C-1), (II-C-2), (II-C-3), (II-C-4), (II-D-1), (II-D-2), (II-D-3), or (II-D-4), or a pharmaceutically acceptable salt, stereoisomer, or deuterated form thereof, L¹ is —(C₁₋₆ alkylene)-NH(CH₂CH₂O)ₘ—(CH₂)ₙ—X^{H}, wherein X^{H} is halogen. In some embodiments, X^{H} is chloro or bromo. In some embodiments, m is an integer of 1-6. In some embodiments, m is an integer of 1-4. In some embodiments, n is an integer of 0-12. In some embodiments, n is an integer of 0-6. In some embodiments, L¹ is —CH₂NH—(CH₂CH₂O)₂—(CH₂)₃—C₁.

In embodiments of the compound of formula (II), (II-A), (II-B), (II-C), (II-D), (II-A-1), (II-A-2), (II-A-3), (II-A-4), (II-B3-1), (II-B3-2), (II-B-3), (II-B-4), (II-C-1), (II-C-2), (II-C-3), (II-C-4), (II-D-1), (II-D-2), (II-D-3), or (II-D-4), or a pharmaceutically acceptable salt, stereoisomer, or deuterated form thereof, L¹ is —O—CH₂CH(OH)—(C₁₋₆ alkylene)-NH(CH₂CH₂O)ₘ—(CH₂)ₙ—C≡CH. In some embodiments, m is an integer of 1-6. In some embodiments, m is an integer of 1-4. In some embodiments, n is an integer of 0-12. In some embodiments, n is an integer of 0-6. In some embodiments, L¹ is —O—CH₂CH(OH)—CH₂—NH(CH₂CH₂O)₂—(CH₂)₂—C≡CH.

In embodiments of the compound of formula (II), (II-A), (II-B), (II-C), (II-D), (II-A-1), (II-A-2), (II-A-3), (II-A-4), (II-B3-1), (II-B3-2), (II-B-3), (II-B-4), (II-C-1), (II-C-2), (II-C-3), (II-C-4), (II-D-1), (II-D-2), (II-D-3), or (II-D-4), or a pharmaceutically acceptable salt, stereoisomer, or deuterated form thereof, L¹ is —O—CH₂CH(OH)—(C₁₋₆ alkylene)-NH(CH₂CH₂O)ₘ—(CH₂)ₙ—C(O)O—(C₁₋₆ alkyl). In some embodiments, m is an integer of 1-6. In some embodiments, m is an integer of 1-4. In some embodiments, n is an integer of 0-12. In some embodiments, n is an integer of 0-6. In some embodiments, L¹ is —O—CH₂CH(OH)—CH₂—NH(CH₂CH₂O)—(CH₂)₂—C(O)OCH₃.

In embodiments of the compound of formula (II), (II-A), (II-B), (II-C), (II-D), (II-A-1), (II-A-2), (II-A-3), (II-A-4), (II-B3-1), (II-B3-2), (II-B-3), (II-B-4), (II-C-1), (II-C-2), (II-C-3), (II-C-4), (II-D-1), (II-D-2), (II-D-3), or (II-D-4), or a pharmaceutically acceptable salt, stereoisomer, or deuterated form thereof, L¹ is —(C₁₋₆ alkylene)-NH(CH₂CH₂O)ₘ—(CH₂)ₙ—(CH₂CH₂O)ₘ—(CH₂)ₙ—C(O)O—(C₁₋₆ alkyl). In some embodiments, each m is independently an integer of 1-12; and each n is independently an integer of 0-12. In some embodiments, each m is an integer of 1-6, 1-4, or 2-3. In some embodiments, each n is an integer of 0-6, 1-5, or 2-3. In some embodiments, L¹ is —CH₂NH(CH₂CH₂O)—(CH₂)—(CH₂CH₂O)—(CH₂)—C(O)OC(CH₃)₃.

In embodiments of the compound of formula (II), (II-A), (II-B), (II-C), (II-D), (II-A-1), (II-A-2), (II-A-3), (II-A-4), (II-B3-1), (II-B3-2), (II-B-3), (II-B-4), (II-C-1), (II-C-2), (II-C-3), (II-C-4), (II-D-1), (II-D-2), (II-D-3), or (II-D-4), or a pharmaceutically acceptable salt, stereoisomer, or deuterated form thereof, wherein L¹ is:
—(C₁₋₆ alkylene)-NH(CH₂CH₂O)ₘ—(CH₂)ₙ—C≡CH,
—O—(C₁₋₆ alkylene)-CH(OH)—(C₁₋₆ alkylene)-NH(CH₂CH₂O)ₘ—(CH₂)ₙ—C≡CH,
—(C₁₋₆ alkylene)-NH(CH₂CH₂O)ₘ—(CH₂)ₙ—NH—C(O)O—(C₁₋₆ alkyl),
—(C₁₋₆ alkylene)-NH(CH₂CH₂O)ₘ—(CH₂)ₙ—N(C₁₋₆ alkyl)-C(O)O—(C₁₋₆ alkyl),
—(C₁₋₆ alkylene)-NH(CH₂CH₂O)ₘ—(CH₂)ₙ—HET^{A}
—(C₁₋₆ alkylene)-NH(CH₂CH₂O)ₘ—(CH₂)ₙ—NH—(C₁₋₆ alkyl),
—(C₁₋₆ alkylene)-NH(CH₂CH₂O)ₘ—(CH₂)ₙ—C(O)O—(C₁₋₆ alkyl),
—O—(C₁₋₆ alkylene)-CH(OH)—(C₁₋₆ alkylene)-NH(CH₂CH₂O)ₘ—(CH₂)ₙ—C(O)O—(C₁₋₆ alkyl), or
—(C₁₋₆ alkylene)-NH(CH₂CH₂O)ₘ—(CH₂)ₙ—O—C₆₋₁₀ aryl;
wherein the alkylene, alkyl, or aryl is optionally and independently substituted with 1, 2, or 3 R^{Z}, wherein each R^{Z} is independently —OH, C₁₋₆ alkyl, or halogen;
HET^{A} is a 4-10 membered heterocycle containing 1-3 N, and is optionally substituted with —C(O)—(C₁₋₆ alkyl), —C(O)O—(C₁₋₆ alkyl), —(C₁₋₆ alkyl)-O—C₆₋₁₀ aryl, or C₁₋₆ alkyl;
m is an integer of 1-12, 2-11, 3-10, 4-9, 5-9, 6-8, or 7; and
n is an integer of 0-6, 1-5, 2-4, or 3.

In embodiments of the compound of formula (II), (II-A), (II-B), (II-C), (II-D), (II-A-1), (II-A-2), (II-A-3), (II-A-4), (II-B3-1), (II-B3-2), (II-B-3), (II-B-4), (II-C-1), (II-C-2), (II-C-3), (II-C-4), (II-D-1), (II-D-2), (II-D-3), or (II-D-4), or a pharmaceutically acceptable salt, stereoisomer, or deuterated form thereof, wherein L¹ is:
—CH₂NH—(CH₂CH₂O)—(CH₂)₂—C≡CH,
—CH₂NH—(CH₂CH₂O)—(CH₂)₅—C≡CH,
—CH₂NH—(CH₂CH₂O)—(CH₂)₆—C≡CH,
—CH₂NH—(CH₂CH₂O)—(CH₂)₈—C≡CH,
—CH₂NH—(CH₂CH₂O)₂—(CH₂)—C≡CH,
—CH₂NH—(CH₂CH₂O)₂—(CH₂)₂—C≡CH,
—CH₂NH—(CH₂CH₂O)₃—(CH₂)—C≡CH,
—CH₂NH—(CH₂CH₂O)₃—(CH₂)₂—C≡CH,

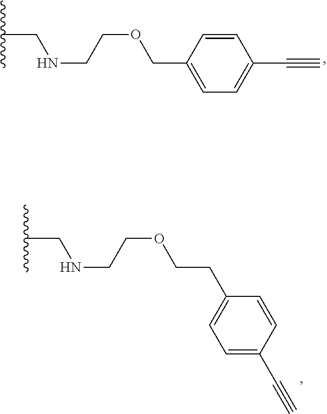 [SMILES: *CNCCOCc1ccc(C#C)cc1 ; *CNCCOCCc1ccc(C#C)cc1]

—O—CH₂—CH(OH)—CH₂—NH—(CH₂CH₂O)₂—(CH₂)₂—C≡CH,
—CH₂NH—(CH₂CH₂O)—(CH₂)₂—NHC(O)OC(CH₃)₃,
—CH₂NH—(CH₂CH₂O)—(CH₂)₃—NHC(O)OC(CH₃)₃,
—CH₂NH—(CH₂CH₂O)—(CH₂)₅—NHC(O)OC(CH₃)₃,
—CH₂NH—(CH₂CH₂O)—(CH₂)₆—NHC(O)OC(CH₃)₃,
—CH₂NH—(CH₂CH₂O)—(CH₂)₈—NHC(O)OC(CH₃)₃,
—CH₂NH—(CH₂CH₂O)₂—(CH₂)₂—NHC(O)OC(CH₃)₃,
—CH₂NH—(CH₂CH₂O)₃—(CH₂)₂—NHC(O)OC(CH₃)₃,

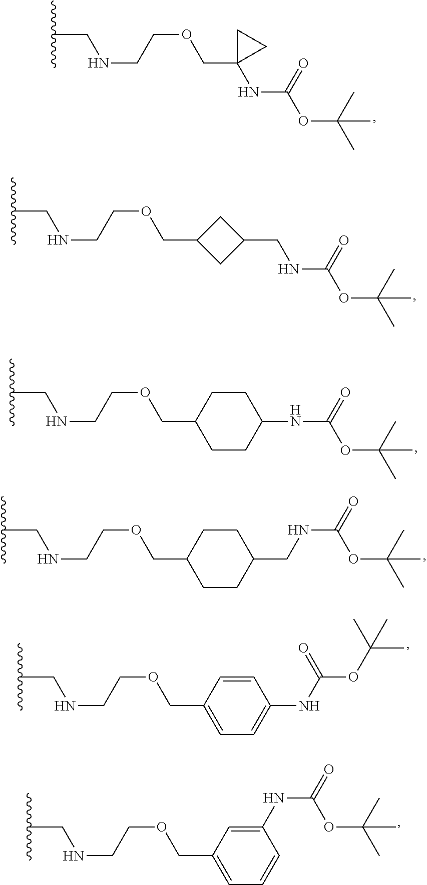

—CH₂NH—(CH₂CH₂O)₃—(CH₂)₃—NCH₃C(O)OC(CH₃)₃,
—CH₂NH—(CH₂CH₂O)₄—(CH₂)₃—NCH₃C(O)OC(CH₃)₃,

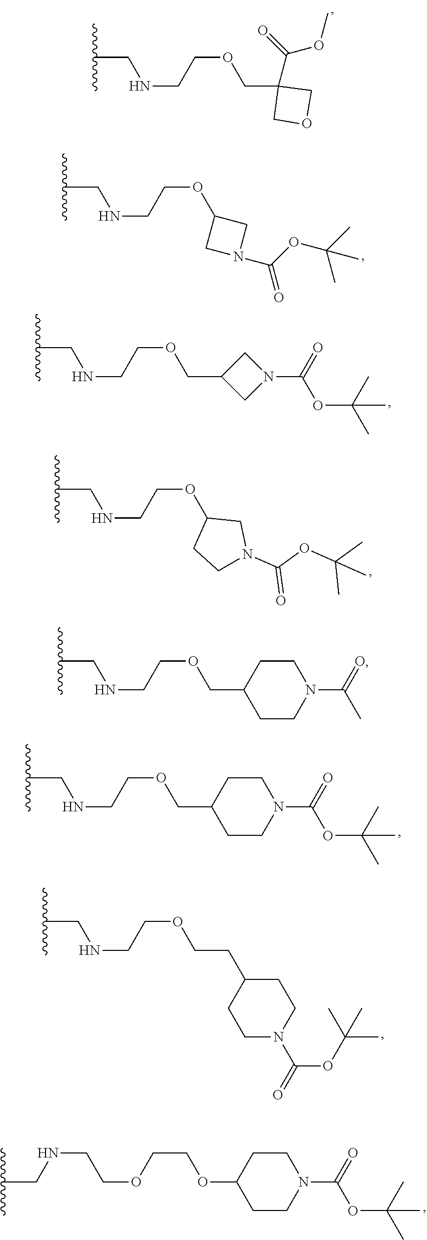

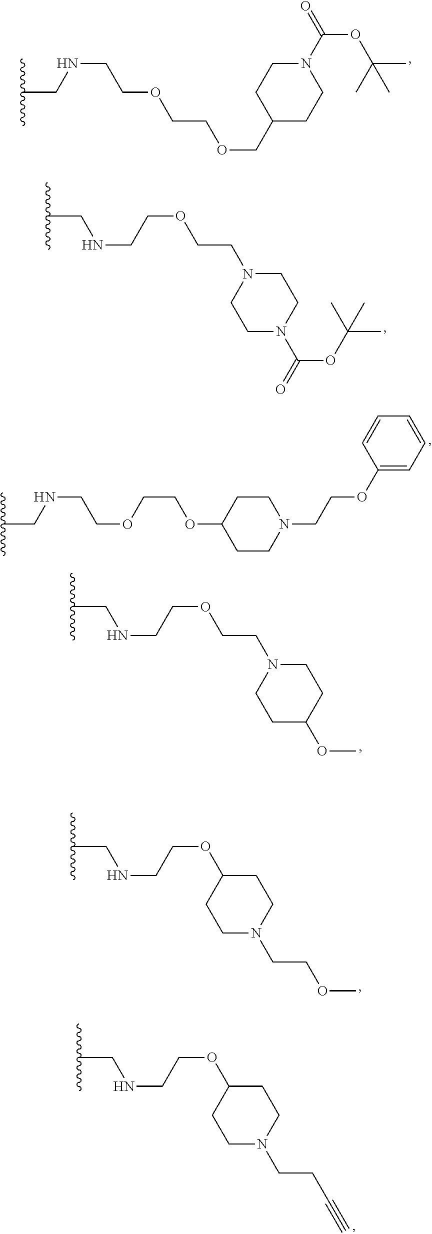 [SMILES: *CNCCOCCOCC1CCN(C(=O)OC(C)(C)C)CC1 ; *CNCCOCCN1CCN(C(=O)OC(C)(C)C)CC1 ; *CNCCOCCOC1CCN(CCOc2ccccc2)CC1 ; *CNCCOCCN1CCC(OC)CC1 ; *CNCCOC1CCN(CCOC)CC1 ; *CNCCOC1CCN(CCC#C)CC1]

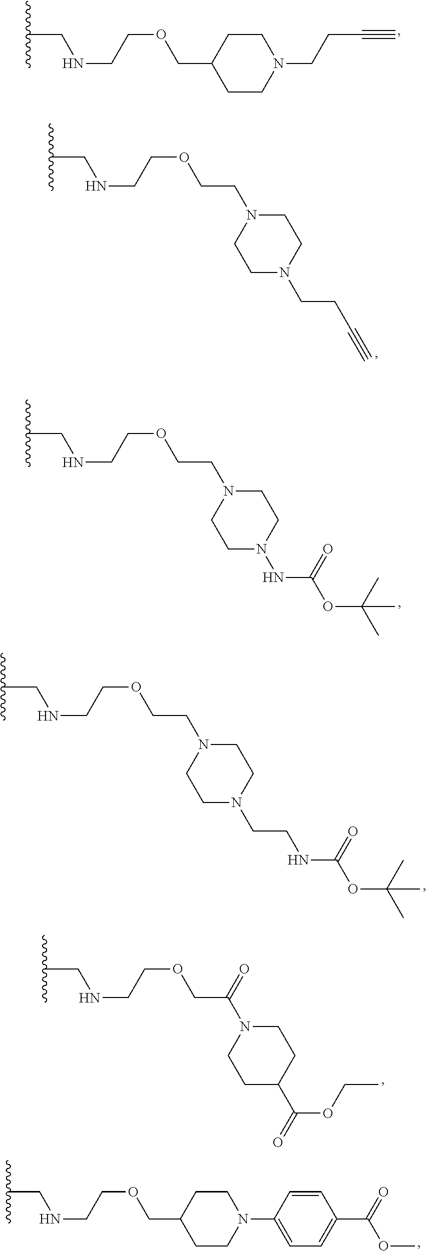 [SMILES: *CNCCOCC1CCN(CCC#C)CC1 ; *CNCCOCCN1CCN(CCC#C)CC1 ; *CNCCOCCN1CCN(NC(=O)OC(C)(C)C)CC1 ; *CNCCOCCN1CCN(CCNC(=O)OC(C)(C)C)CC1 ; *CNCCOCC(=O)N1CCC(C(=O)OCC)CC1 ; *CNCCOCC1CCN(c2ccc(C(=O)OC)cc2)CC1]

—CH₂NH—(CH₂CH₂O)₂—(CH₂)₂—NHCH₃,
—CH₂NH—(CH₂CH₂O)₂—(CH₂)₃—NHCH₃,
—CH₂NH—(CH₂CH₂O)₃—(CH₂)₂—NHCH₃,
—CH₂NH—(CH₂CH₂O)₂—(CH₂)₂—C(O)NH—CH₃,
—CH₂NH—(CH₂CH₂O)—(CH₂)—C(O)NH—(CH₂)—C(O)OCH₂CH₃,
—CH₂NH—(CH₂CH₂O)₂—(CH₂)₂—C(O)OCH₃,
—CH₂NH—(CH₂CH₂O)₃—(CH₂)₂—C(O)OCH₃,

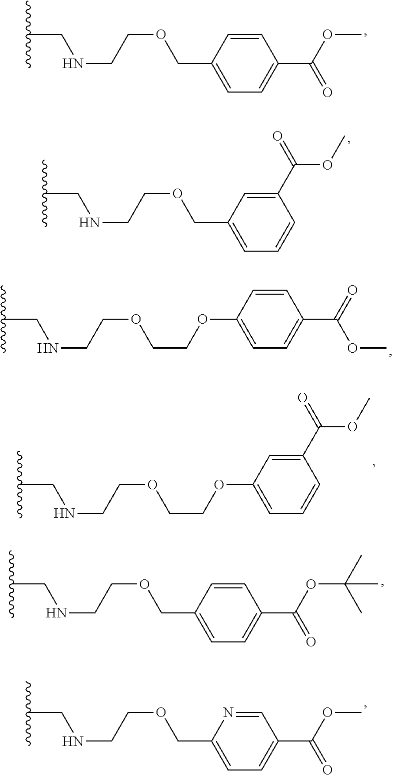

—CH₂NH(CH₂CH₂O)—(CH₂)—C(O)OC(CH₃)₃,
—CH₂NH(CH₂CH₂O)—(CH₂)₂—C(O)OC(CH₃)₃,
—CH₂NH(CH₂CH₂O)—(CH₂)₃—C(O)OC(CH₃)₃,
—CH₂NH(CH₂CH₂O)—(CH₂)₄—C(O)OC(CH₃)₃,
—CH₂NH(CH₂CH₂O)—(CH₂)₅—C(O)OC(CH₃)₃,
—CH₂NH(CH₂CH₂O)—(CH₂)₈—C(O)OC(CH₃)₃,
—CH₂NH(CH₂CH₂O)₂—(CH₂)₂—C(O)OC(CH₃)₃,
—CH₂NH(CH₂CH₂O)—(CH₂)—(CH₂CH₂O)—(CH₂)—C(O)OC(CH₃)₃,
—O—CH₂—CH(OH)—CH₂NH—(CH₂CH₂O)—(CH₂)₂—C(O)OCH₃,

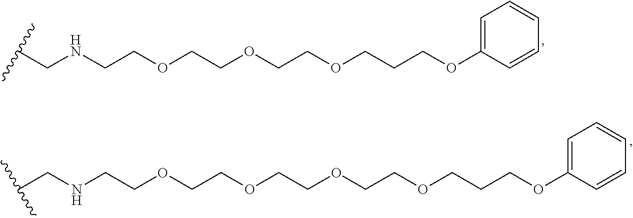 [SMILES: *CNCCOCCOCCOCCCOc1ccccc1 ; *CNCCOCCOCCOCCOCCCOc1ccccc1]

—CH₂NH—(CH₂CH₂O)—(CH₂)₃—C₁, or
—O—CH₂CH(OH)—CH₂—NH(CH₂CH₂O)—(CH₂)₂—C(O)OCH₃.

In embodiments of the compound of formula (II), (II-A), (II-B), (II-C), (II-D), (II-A-1), (II-A-2), (II-A-3), (II-A-4), (II-B3-1), (II-B3-2), (II-B-3), (II-B-4), (II-C-1), (II-C-2), (II-C-3), (II-C-4), (II-D-1), (II-D-2), (II-D-3), or (II-D-4), or a pharmaceutically acceptable salt, stereoisomer, or deuterated form thereof, wherein L¹ is
—CH₂NH—(CH₂CH₂O)₂—(CH₂)₂—C≡CH,
—CH₂NH—(CH₂CH₂O)₃—(CH₂)₂—C≡CH,
—O—CH₂—CH(OH)—CH₂—NH—(CH₂CH₂O)₂—(CH₂)₂—C≡CH,
—CH₂NH—(CH₂CH₂O)₃—(CH₂)₂—NHC(O)OC(CH₃)₃,
—CH₂NH—(CH₂CH₂O)₃—(CH₂)₃—NCH₃C(O)OC(CH₃)₃,
—CH₂NH—(CH₂CH₂O)₄—(CH₂)₃—NCH₃C(O)OC(CH₃)₃,

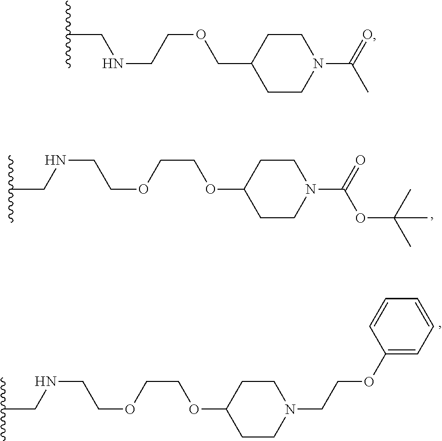 [SMILES: *CNCCOCC1CCN(C(C)=O)CC1 ; *CNCCOCCOC1CCN(C(=O)OC(C)(C)C)CC1 ; *CNCCOCCOC1CCN(CCOc2ccccc2)CC1]

—CH₂NH—(CH₂CH₂O)₂—(CH₂)₂—NHCH₃,
—CH₂NH—(CH₂CH₂O)₃—(CH₂)₂—NHCH₃,
—CH₂NH—(CH₂CH₂O)₂—(CH₂)₂—C(O)OCH₃,
—O—CH₂—CH(OH)—CH₂NH—(CH₂CH₂O)—(CH₂)₂—C(O)OCH₃,

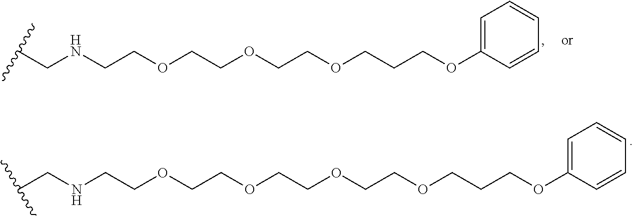 [SMILES: *CNCCOCCOCCOCCCOc1ccccc1 ; *CNCCOCCOCCOCCOCCCOc1ccccc1]

In embodiments of the compound of formula (II), (II-A), (II-B), (II-C), (II-D), (II-A-1), (II-A-2), (II-A-3), (II-A-4), (II-B3-1), (II-B3-2), (II-B-3), (II-B-4), (II-C-1), (II-C-2), (II-C-3), (II-C-4), (II-D-1), (II-D-2), (II-D-3), or (II-D-4), or a pharmaceutically acceptable salt, stereoisomer, or deuterated form thereof, wherein L¹ comprises a bivalent moiety selected from:

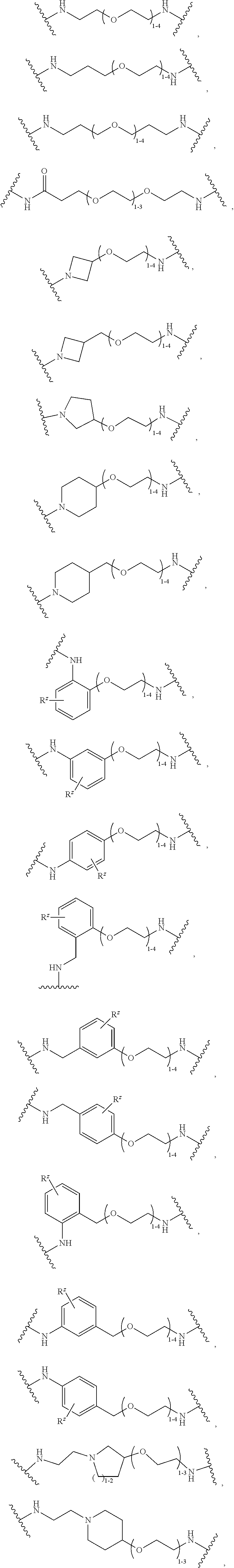

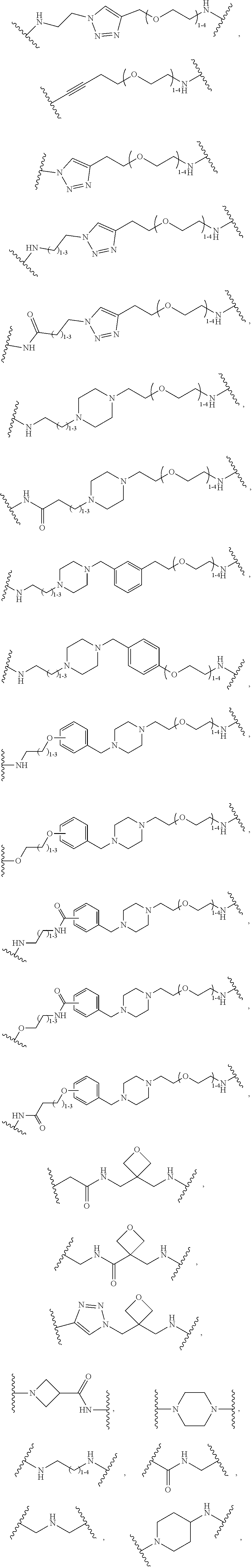

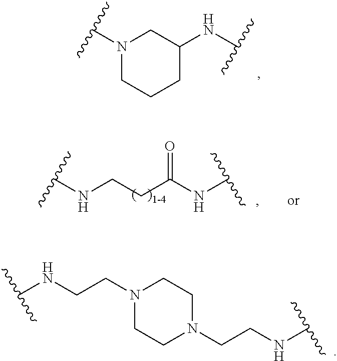

In embodiments of the compound of formula (II), (II-A), (II-B), (II-C), (II-D), (II-A-1), (II-A-2), (II-A-3), (II-A-4), (II-B3-1), (II-B3-2), (II-B-3), (II-B-4), (II-C-1), (II-C-2), (II-C-3), (II-C-4), (II-D-1), (II-D-2), (II-D-3), or (II-D-4), or a pharmaceutically acceptable salt, stereoisomer, or deuterated form thereof, wherein L¹ comprises a bivalent moiety selected from:

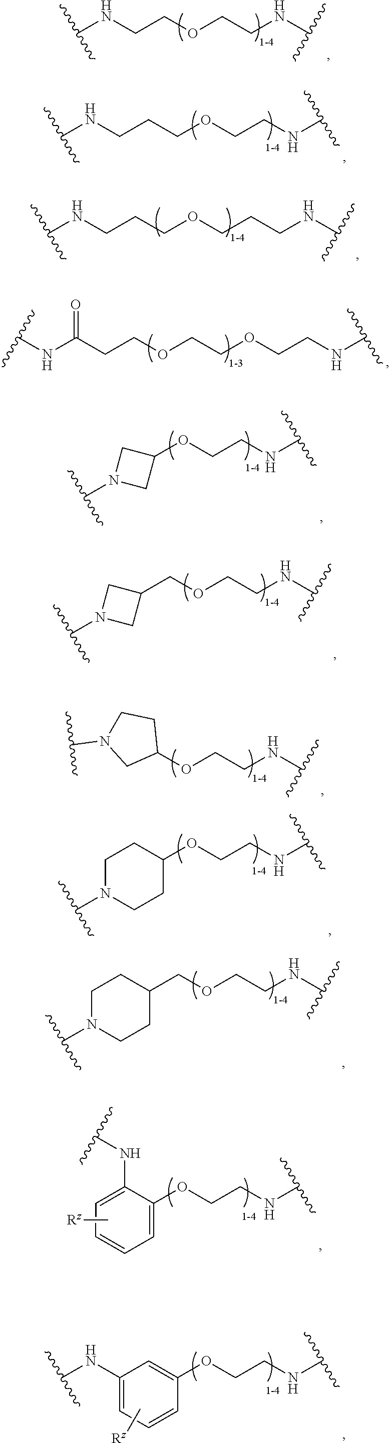

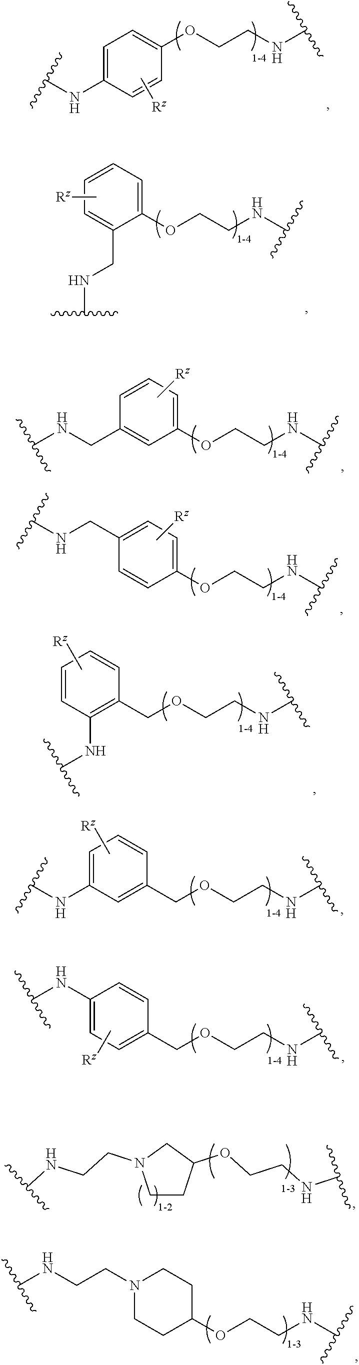

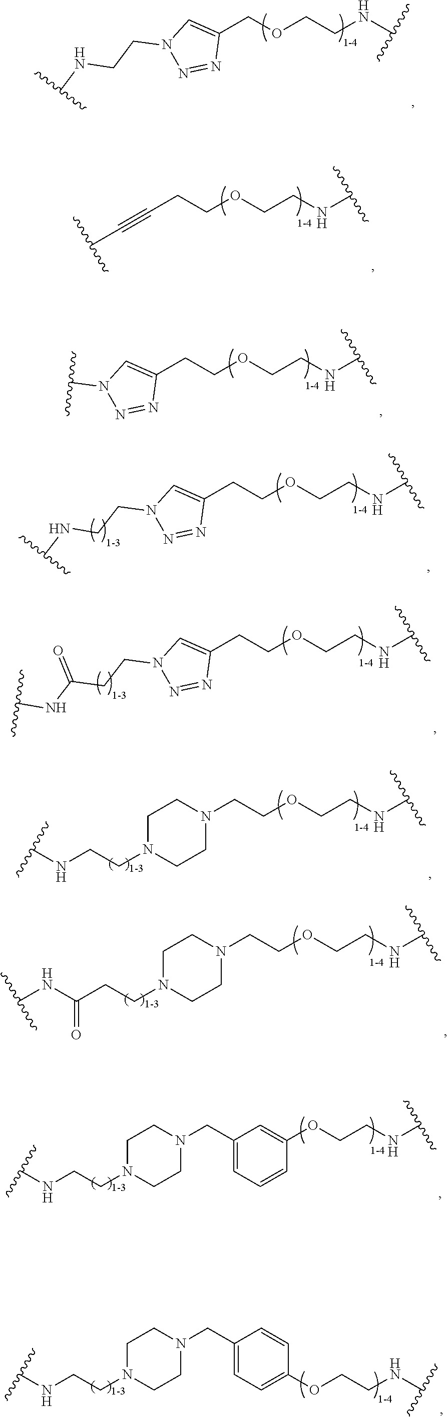

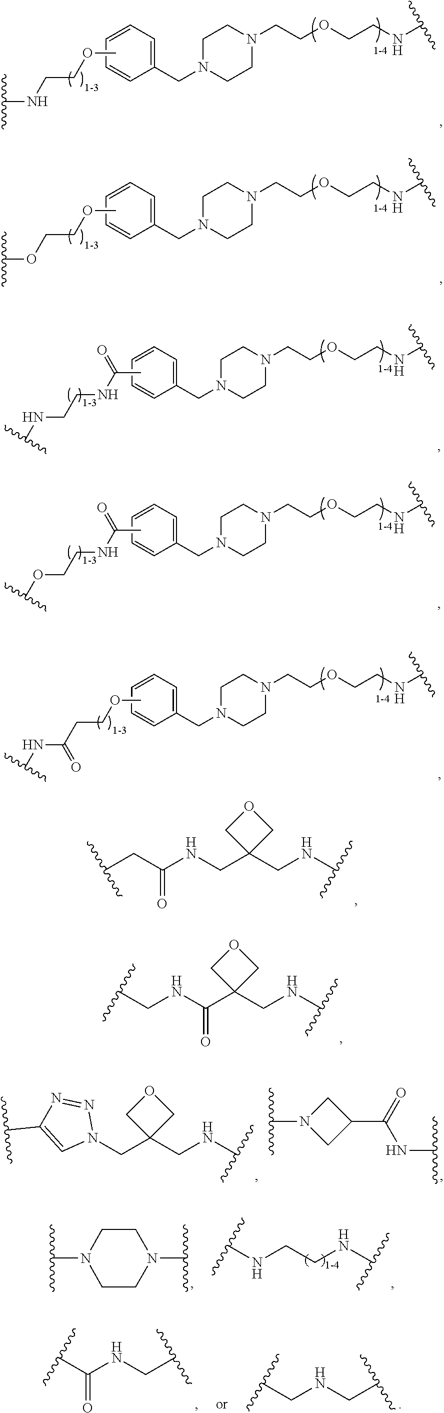

In embodiments, a ligand (“A”) that binds to a protein, a protein aggregate, a protein complex, or a lipid that is targeted for degradation is covalently attached to the compounds of formula (X-II), (II), (II-A), (II-B3), (II-C), (II-D), (II-A-1), (II-A-2), (II-A-3), (II-A-4), (II-B3-1), (II-B3-2), (II-B3-3), (II-B3-4), (II-C-1), (II-C-2), (II-C-3), (II-C-4), (II-D-1), (II-D-2), (II-D-3), or (II-D-4). In embodiments, the present disclosure provides a compound of formula (X-III):

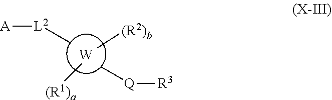

or a pharmaceutically acceptable salt, stereoisomer, or deuterated form thereof,
wherein:
A is a ligand (e.g., a PBC) that binds to a protein, a protein aggregate, a protein complex, or a lipid;
ring W is 6,5-fused heteroaryl or 6,5-fused heterocycle ring, wherein each ring W contains 1, 2, or 3 heteroatoms selected from N, O, or S, and at least 1 of the heteroatoms is N or O;
each R¹ is independently C₁₋₆ alkyl, C₃-C₈ cycloalkyl, or halogen;
each R² is independently C₁₋₆ alkyl, C₃-C₈ cycloalkyl, or halogen, or two R² form an oxo;
Q is absent, C₁₋₆ alkylene, —C₁₋₆ alkylene-C(O)—, C₃₋₈ cycloalkylene, —C₃₋₈ cycloalkylene-C(O)—, —C(O)—, or —S(O)₂—;
R³ is carbocycle, heterocycle, aryl, heteroaryl, or —NR^{X}R^{Y}, wherein the carbocycle, heterocycle, aryl, and heteroaryl are each optionally substituted with 1, 2, or 3 R⁴;
each R⁴ is independently halogen, —OH, C₁₋₆ alkyl, C₁₋₆ alkoxy, —C(O)—NR^{X}R^{Y}, or —C(O)O—(C₁₋₆ alkyl)
R^{X} is H, C₁₋₆ alkyl, C₁₋₆ haloalkyl, or C₁₋₆ hydroxyalkyl;
R^{Y} is C₁₋₆ alkyl, C₃₋₈ cycloalkyl, C₃₋₈ hydroxycycloalkyl, aryl, or heterocycle
a is an integer of 0-3;
b is an integer of 0-2; and
L² is a linker moiety that covalently binds ligand A to ring W.

In some embodiments, L² is C₂-C₅₀ alkylene, C₂-C₂₅ alkenylene, C₂-C₂₅ alkynylene, or —(C₂-C₅₀ alkylene)-arylene, wherein 1-25 methylene groups of L² are optionally and independently replaced by —N(H)—, —N(C₁-C₆ alkyl)-, —N(C₃-C₈ cycloalkyl)-, —O—, —C(O)—, —C(O)O—, —S—, —S(O)—, —S(O)₂—, —S(O)₂N(C₁-C₆ alkyl)-, —S(O)₂N(C₃-C₈ cycloalkyl)-, —N(H)C(O)—, —N(C₁-C₆ alkyl)C(O)—, —N(C₃-C₈ cycloalkyl)C(O)—, —N(H)C(O)N(H)—, —N(C₁-C₆ alkyl)C(O)N(H)—, —N(H)C(O)N(C₁-C₆ alkyl)-, —N(C₁-C₆ alkyl)C(O)N(C₁-C₆ alkyl)-, —C(O)N(H)—, —C(O)N(C₁-C₆ alkyl)-, —C(O)N(C₃-C₈ cycloalkyl)-, arylene, heteroarylene, heterocyclylene, C₃-C₈ cycloalkylene, or C₃-C₈ cycloalkenylene, wherein the alkylene, alkenylene, alkynylene, alkyl, arylene, heteroarylene, and heterocyclylene are each optionally and independently substituted with 1, 2, or 3 R^{Z}, wherein each R^{Z} is independently OH, C₁₋₆ alkyl, or halogen.

In embodiments, the present disclosure provides a compound of formula (III)

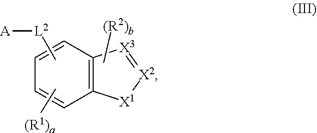

or a pharmaceutically acceptable salt, stereoisomer, or deuterated form thereof,
wherein:
A is a ligand that binds to a protein, a protein aggregate, a protein complex, or a lipid;
L² is C₂-C₅₀ alkylene, C₂-C₂₅ alkenylene, C₂-C₂₅ alkynylene, or —(C₂-C₅₀ alkylene)-arylene, wherein 1-25 methylene groups of L² are optionally and independently replaced by —N(H)—, —N(C₁-C₆ alkyl)-, —N(C₃-C₈ cycloalkyl)-, —O—, —C(O)—, —C(O)O—, —S—, —S(O)—, —S(O)₂—, —S(O)₂N(C₁-C₆ alkyl)-, —S(O)₂N(C₃-C₈ cycloalkyl)-, —N(H)C(O)—, —N(C₁-C₆ alkyl)C(O)—, —N(C₃-C₈ cycloalkyl)C(O)—, —N(H)C(O)N(H)—, —N(C₁-C₆ alkyl)C(O)N(H)—, —N(H)C(O)N(C₁-C₆ alkyl)-, —N(C₁-C₆ alkyl)C(O)N(C₁-C₆ alkyl)-, —C(O)N(H)—, —C(O)N(C₁-C₆ alkyl)-, —C(O)N(C₃-C₈ cycloalkyl)-, arylene, heteroarylene, heterocyclylene, C₃-C₈ cycloalkylene, or C₃-C₈ cycloalkenylene, wherein the alkylene, alkenylene, alkynylene, alkyl, arylene, heteroarylene, and heterocyclylene are each optionally and independently substituted with 1, 2, or 3 R^{Z}, wherein each R^{Z} is independently OH, C₁₋₆ alkyl, or halogen;
each R¹ is independently C₁₋₆ alkyl, C₃-C₈ cycloalkyl, or halogen;
each R² is independently C₁₋₆ alkyl, C₃-C₈ cycloalkyl, or halogen;
X¹ is O or S;
one of X² or X³ is C-Q-R³ and the other is CH, C, or N as permitted by valency;
Q is absent, C₁₋₆ alkylene, —C₁₋₆ alkylene-C(O)—, C₃₋₈ cycloalkylene, —C₃₋₈ cycloalkylene-C(O)—, —C(O)—, or —S(O)₂—;
R³ is carbocycle, heterocycle, aryl, heteroaryl, or —NR^{X}R^{Y}, wherein the carbocycle, heterocycle, aryl, and heteroaryl are each optionally substituted with 1, 2, or 3 R⁴;
each R⁴ is independently halogen, —OH, C₁₋₆ alkyl, C₁₋₆ alkoxy, —C(O)—NR^{X}R^{Y}, or —C(O)O—(C₁₋₆ alkyl);
R^{X} is H, C₁₋₆ alkyl, C₁₋₆ haloalkyl, or C₁₋₆ hydroxyalkyl;
R^{Y} is C₁₋₆ alkyl, C₃₋₈ cycloalkyl, C₃₋₈ hydroxycycloalkyl, aryl, or heterocycle;
a is an integer of 0-3; and
b is an integer of 0 or 1.

In embodiments, the present disclosure provides a compound of formula (III-A): (III-A):

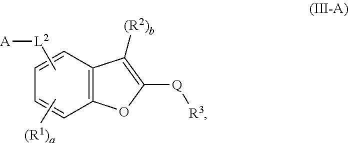

or a pharmaceutically acceptable salt, a stereoisomer, or a deuterated form thereof,
wherein:
A is a ligand that binds to a protein, a protein aggregate, a protein complex, or a lipid;
L² is C₂-C₅₀ alkylene, C₂-C₂₅ alkenylene, C₂-C₂₅ alkynylene, or —(C₂-C₅₀ alkylene)-arylene, wherein 1-25 methylene groups of L² are optionally and independently replaced by —N(H)—, —N(C₁-C₆ alkyl)-, —N(C₃-C₈ cycloalkyl)-, —O—, —C(O)—, —C(O)O—, —S—, —S(O)—, —S(O)₂—, —S(O)₂N(C₁-C₆ alkyl)-, —S(O)₂N(C₃-C₈ cycloalkyl)-, —N(H)C(O)—, —N(C₁-C₆ alkyl)C(O)—, —N(C₃-C₈ cycloalkyl)C(O)—, —N(H)C(O)N(H)—, —N(C₁-C₆ alkyl)C(O)N(H)—, —N(H)C(O)N(C₁-C₆ alkyl)-, —N(C₁-C₆ alkyl)C(O)N(C₁-C₆ alkyl)-, —C(O)N(H)—, —C(O)N(C₁-C₆ alkyl)-, —C(O)N(C₃-C₈ cycloalkyl)-, arylene, heteroarylene, heterocyclylene, C₃-C₈ cycloalkylene, or C₃-C₈ cycloalkenylene, wherein the alkylene, alkenylene, alkynylene, alkyl, arylene, heteroarylene, and heterocyclylene are each optionally and independently substituted with 1, 2, or 3 R^{Z}, wherein each R^{Z} is independently OH, C₁₋₆ alkyl, or halogen;
each R¹ is independently C₁₋₆ alkyl, C₃-C₈ cycloalkyl, or halogen;
each R² is independently C₁₋₆ alkyl, C₃-C₈ cycloalkyl, or halogen;
Q is absent, C₁₋₆ alkylene, —C₁₋₆ alkylene-C(O)—, C₃₋₈ cycloalkylene, —C₃₋₈ cycloalkylene-C(O)—, —C(O)—, or —S(O)₂—;
R³ is carbocycle, heterocycle, aryl, heteroaryl, or —NR^{X}R^{Y}, wherein the carbocycle, heterocycle, aryl, and heteroaryl are each optionally substituted with 1, 2, or 3 R⁴;
each R⁴ is independently halogen, —OH, C₁₋₆ alkyl, C₁₋₆ alkoxy, —C(O)—NR^{X}R^{Y}, or —C(O)O—(C₁₋₆ alkyl);
R^{X} is H, C₁₋₆ alkyl, C₁₋₆ haloalkyl, or C₁₋₆ hydroxyalkyl;
R^{Y} is C₁₋₆ alkyl, C₃₋₈ cycloalkyl, C₃₋₈ hydroxycycloalkyl, aryl, or heterocycle;
a is an integer of 0-3; and
b is an integer of 0 or 1.

In embodiments, the present disclosure provides a compound of formula (III-A-1), (III-A-2), (III-A-3), or (III-A-4):

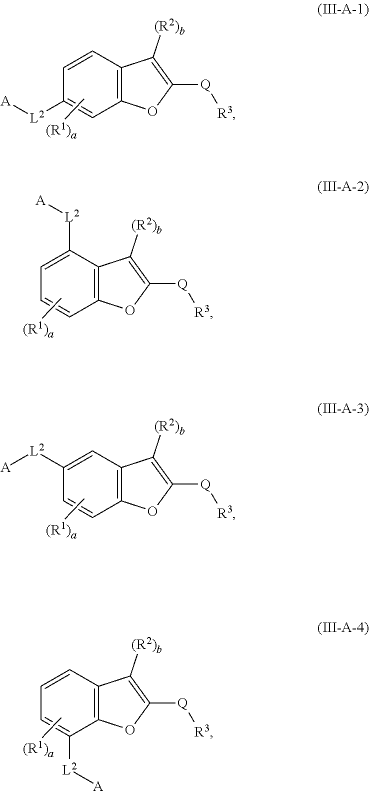

or a pharmaceutically acceptable salt, a stereoisomer, or a deuterated form thereof,
wherein:
A is a ligand that binds to a protein, a protein aggregate, a protein complex, or a lipid;
L² is C₂-C₅₀ alkylene, C₂-C₂₅ alkenylene, C₂-C₂₅ alkynylene, or —(C₂-C₅₀ alkylene)-arylene, wherein 1-25 methylene groups of L² are optionally and independently replaced by —N(H)—, —N(C₁-C₆ alkyl)-, —N(C₃-C₈ cycloalkyl)-, —O—, —C(O)—, —C(O)O—, —S—, —S(O)—, —S(O)₂—, —S(O)₂N(C₁-C₆ alkyl)-, —S(O)₂N(C₃-C₈ cycloalkyl)-, —N(H)C(O)—, —N(C₁-C₆ alkyl)C(O)—, —N(C₃-C₈ cycloalkyl)C(O)—, —N(H)C(O)N(H)—, —N(C₁-C₆ alkyl)C(O)N(H)—, —N(H)C(O)N(C₁-C₆ alkyl)-, —N(C₁-C₆ alkyl)C(O)N(C₁-C₆ alkyl)-, —C(O)N(H)—, —C(O)N(C₁-C₆ alkyl)-, —C(O)N(C₃-C₈ cycloalkyl)-, arylene, heteroarylene, heterocyclylene, C₃-C₈ cycloalkylene, or C₃-C₈ cycloalkenylene, wherein the alkylene, alkenylene, alkynylene, alkyl, arylene, heteroarylene, and heterocyclylene are each optionally and independently substituted with 1, 2, or 3 R^{Z}, wherein each R^{Z} is independently OH, C₁₋₆ alkyl, or halogen;
each R¹ is independently C₁₋₆ alkyl, C₃-C₈ cycloalkyl, or halogen;
each R² is independently C₁₋₆ alkyl, C₃-C₈ cycloalkyl, or halogen;
Q is absent, C₁₋₆ alkylene, —C₁₋₆ alkylene-C(O)—, C₃₋₈ cycloalkylene, —C₃₋₈ cycloalkylene-C(O)—, —C(O)—, or —S(O)₂—;
R³ is carbocycle, heterocycle, aryl, heteroaryl, or —NR^{X}R^{Y}, wherein the carbocycle, heterocycle, aryl, and heteroaryl are each optionally substituted with 1, 2, or 3 R⁴;
each R⁴ is independently halogen, —OH, C₁₋₆ alkyl, C₁₋₆ alkoxy, —C(O)—NR^{X}R^{Y}, or —C(O)O—(C₁₋₆ alkyl);
R^{X} is H, C₁₋₆ alkyl, C₁₋₆ haloalkyl, or C₁₋₆ hydroxyalkyl;
R^{Y} is C₁₋₆ alkyl, C₃₋₈ cycloalkyl, C₃₋₈ hydroxycycloalkyl, aryl, or heterocycle;
a is an integer of 0-3; and
b is an integer of 0 or 1.

In embodiments, the present disclosure provides a compound of formula (III-B):

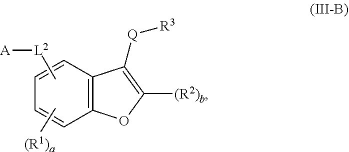

or a pharmaceutically acceptable salt, a stereoisomer, or a deuterated form thereof,
wherein:
A is a ligand that binds to a protein, a protein aggregate, a protein complex, or a lipid;
L² is C₂-C₅₀ alkylene, C₂-C₂₅ alkenylene, C₂-C₂₅ alkynylene, or —(C₂-C₅₀ alkylene)-arylene, wherein 1-25 methylene groups of L² are optionally and independently replaced by —N(H)—, —N(C₁-C₆ alkyl)-, —N(C₃-C₈ cycloalkyl)-, —O—, —C(O)—, —C(O)O—, —S—, —S(O)—, —S(O)₂—, —S(O)₂N(C₁-C₆ alkyl)-, —S(O)₂N(C₃-C₈ cycloalkyl)-, —N(H)C(O)—, —N(C₁-C₆ alkyl)C(O)—, —N(C₃-C₈ cycloalkyl)C(O)—, —N(H)C(O)N(H)—, —N(C₁-C₆ alkyl)C(O)N(H)—, —N(H)C(O)N(C₁-C₆ alkyl)-, —N(C₁-C₆ alkyl)C(O)N(C₁-C₆ alkyl)-, —C(O)N(H)—, —C(O)N(C₁-C₆ alkyl)-, —C(O)N(C₃-C₈ cycloalkyl)-, arylene, heteroarylene, heterocyclylene, C₃-C₈ cycloalkylene, or C₃-C₈ cycloalkenylene, wherein the alkylene, alkenylene, alkynylene, alkyl, arylene, heteroarylene, and heterocyclylene are each optionally and independently substituted with 1, 2, or 3 R^{Z}, wherein each R^{Z} is independently OH, C₁₋₆ alkyl, or halogen;
each R¹ is independently C₁₋₆ alkyl, C₃-C₈ cycloalkyl, or halogen;
each R² is independently C₁₋₆ alkyl, C₃-C₈ cycloalkyl, or halogen;
Q is absent, C₁₋₆ alkylene, —C₁₋₆ alkylene-C(O)—, C₃₋₈ cycloalkylene, —C₃₋₈ cycloalkylene-C(O)—, —C(O)—, or —S(O)₂—;
R³ is carbocycle, heterocycle, aryl, heteroaryl, or —NR^{X}R^{Y}, wherein the carbocycle, heterocycle, aryl, and heteroaryl are each optionally substituted with 1, 2, or 3 R⁴;
each R⁴ is independently halogen, —OH, C₁₋₆ alkyl, C₁₋₆ alkoxy, —C(O)—NR^{X}R^{Y}, or —C(O)O—(C₁₋₆ alkyl);
R^{X} is H, C₁₋₆ alkyl, C₁₋₆ haloalkyl, or C₁₋₆ hydroxyalkyl;
R^{Y} is C₁₋₆ alkyl, C₃₋₈ cycloalkyl, C₃₋₈ hydroxycycloalkyl, aryl, or heterocycle;
a is an integer of 0-3; and
b is an integer of 0 or 1.

In embodiments, the present disclosure provides a compound of formula (III-B-1), (III-B-2), (III-B-3), or (III-B-4):

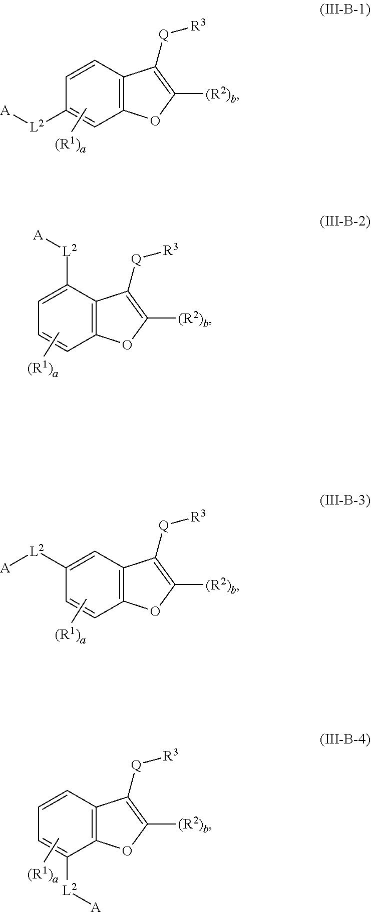

or a pharmaceutically acceptable salt, a stereoisomer, or a deuterated form thereof,
wherein:
A is a ligand that binds to a protein, a protein aggregate, a protein complex, or a lipid;
L² is C₂-C₅₀ alkylene, C₂-C₂₅ alkenylene, C₂-C₂₅ alkynylene, or —(C₂-C₅₀ alkylene)-arylene, wherein 1-25 methylene groups of L² are optionally and independently replaced by —N(H)—, —N(C₁-C₆ alkyl)-, —N(C₃-C₈ cycloalkyl)-, —O—, —C(O)—, —C(O)O—, —S—, —S(O)—, —S(O)₂—, —S(O)₂N(C₁-C₆ alkyl)-, —S(O)₂N(C₃-C₈ cycloalkyl)-, —N(H)C(O)—, —N(C₁-C₆ alkyl)C(O)—, —N(C₃-C₈ cycloalkyl)C(O)—, —N(H)C(O)N(H)—, —N(C₁-C₆ alkyl)C(O)N(H)—, —N(H)C(O)N(C₁-C₆ alkyl)-, —N(C₁-C₆ alkyl)C(O)N(C₁-C₆ alkyl)-, —C(O)N(H)—, —C(O)N(C₁-C₆ alkyl)-, —C(O)N(C₃-C₈ cycloalkyl)-, arylene, heteroarylene, heterocyclylene, C₃-C₈ cycloalkylene, or C₃-C₈ cycloalkenylene, wherein the alkylene, alkenylene, alkynylene, alkyl, arylene, heteroarylene, and heterocyclylene are each optionally and independently substituted with 1, 2, or 3 R^{Z}, wherein each R^{Z} is independently OH, C₁₋₆ alkyl, or halogen;
each R¹ is independently C₁₋₆ alkyl, C₃-C₈ cycloalkyl, or halogen;
each R² is independently C₁₋₆ alkyl, C₃-C₈ cycloalkyl, or halogen;
Q is absent, C₁₋₆ alkylene, —C₁₋₆ alkylene-C(O)—, C₃₋₈ cycloalkylene, —C₃₋₈ cycloalkylene-C(O)—, —C(O)—, or —S(O)₂—;
R³ is carbocycle, heterocycle, aryl, heteroaryl, or —NR^{X}R^{Y}, wherein the carbocycle, heterocycle, aryl, and heteroaryl are each optionally substituted with 1, 2, or 3 R⁴;
each R⁴ is independently halogen, —OH, C₁₋₆ alkyl, C₁₋₆ alkoxy, —C(O)—NR^{X}R^{Y}, or —C(O)O—(C₁₋₆ alkyl);
R^{X} is H, C₁₋₆ alkyl, C₁₋₆ haloalkyl, or C₁₋₆ hydroxyalkyl;
R^{Y} is C₁₋₆ alkyl, C₃₋₈ cycloalkyl, C₃₋₈ hydroxycycloalkyl, aryl, or heterocycle;
a is an integer of 0-3; and
b is an integer of 0 or 1.

In embodiments, the present disclosure provides a compound of formula (III-C) or (III-D):

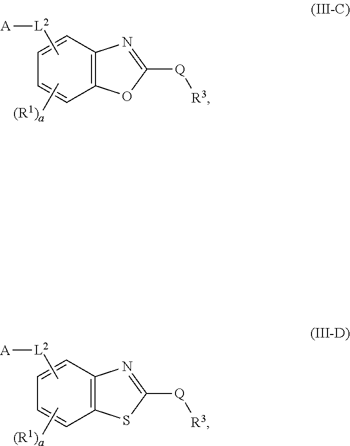

or a pharmaceutically acceptable salt, a stereoisomer, or a deuterated form thereof,
wherein:
A is a ligand that binds to a protein, a protein aggregate, a protein complex, or a lipid;
L² is C₂-C₅₀ alkylene, C₂-C₂₅ alkenylene, C₂-C₂₅ alkynylene, or —(C₂-C₅₀ alkylene)-arylene, wherein 1-25 methylene groups of L² are optionally and independently replaced by —N(H)—, —N(C₁-C₆ alkyl)-, —N(C₃-C₈ cycloalkyl)-, —O—, —C(O)—, —C(O)O—, —S—, —S(O)—, —S(O)₂—, —S(O)₂N(C₁-C₆ alkyl)-, —S(O)₂N(C₃-C₈ cycloalkyl)-, —N(H)C(O)—, —N(C₁-C₆ alkyl)C(O)—, —N(C₃-C₈ cycloalkyl)C(O)—, —N(H)C(O)N(H)—, —N(C₁-C₆ alkyl)C(O)N(H)—, —N(H)C(O)N(C₁-C₆ alkyl)-, —N(C₁-C₆ alkyl)C(O)N(C₁-C₆ alkyl)-, —C(O)N(H)—, —C(O)N(C₁-C₆ alkyl)-, —C(O)N(C₃-C₈ cycloalkyl)-, arylene, heteroarylene, heterocyclylene, C₃-C₈ cycloalkylene, or C₃-C₈ cycloalkenylene, wherein the alkylene, alkenylene, alkynylene, alkyl, arylene, heteroarylene, and heterocyclylene are each optionally and independently substituted with 1, 2, or 3 R^{Z}, wherein each R^{Z} is independently OH, C₁₋₆ alkyl, or halogen;
each R¹ is independently C₁₋₆ alkyl, C₃-C₈ cycloalkyl, or halogen;
Q is absent, C₁₋₆ alkylene, —C₁₋₆ alkylene-C(O)—, C₃₋₈ cycloalkylene, —C₃₋₈ cycloalkylene-C(O)—, —C(O)—, or —S(O)₂—;
R³ is carbocycle, heterocycle, aryl, heteroaryl, or —NR^{X}R^{Y}, wherein the carbocycle, heterocycle, aryl, and heteroaryl are each optionally substituted with 1, 2, or 3 R⁴;
each R⁴ is independently halogen, —OH, C₁₋₆ alkyl, C₁₋₆ alkoxy, —C(O)—NR^{X}R^{Y}, or —C(O)O—(C₁₋₆ alkyl);
R^{X} is H, C₁₋₆ alkyl, C₁₋₆ haloalkyl, or C₁₋₆ hydroxyalkyl;
R^{Y} is C₁₋₆ alkyl, C₃₋₈ cycloalkyl, C₃₋₈ hydroxycycloalkyl, aryl, or heterocycle; and
a is an integer of 0-3.

In embodiments, the present disclosure provides a compound of formula (III-A) formula (III-C-1), (III-C-2), (III-C-3), (III-C-4), (III-D-1), (III-D-2), (III-D-3), or (III-D-4):

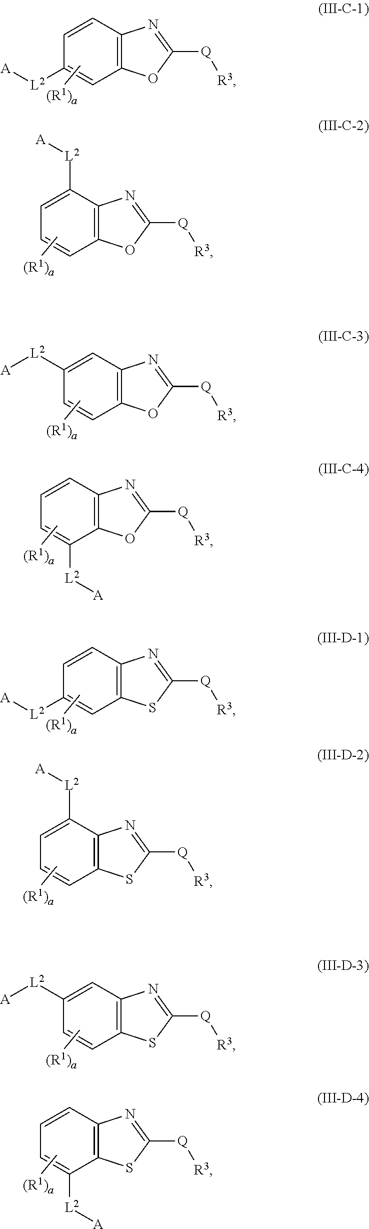

or a pharmaceutically acceptable salt, a stereoisomer, or a deuterated form thereof,
wherein:
A is a ligand that binds to a protein, a protein aggregate, a protein complex, or a lipid;
L² is C₂-C₅₀ alkylene, C₂-C₂₅ alkenylene, C₂-C₂₅ alkynylene, or —(C₂-C₅₀ alkylene)-arylene, wherein 1-25 methylene groups of L² are optionally and independently replaced by —N(H)—, —N(C₁-C₆ alkyl)-, —N(C₃-C₈ cycloalkyl)-, —O—, —C(O)—, —C(O)O—, —S—, —S(O)—, —S(O)₂—, —S(O)₂N(C₁-C₆ alkyl)-, —S(O)₂N(C₃-C₈ cycloalkyl)-, —N(H)C(O)—, —N(C₁-C₆ alkyl)C(O)—, —N(C₃-C₈ cycloalkyl)C(O)—, —N(H)C(O)N(H)—, —N(C₁-C₆ alkyl)C(O)N(H)—, —N(H)C(O)N(C₁-C₆ alkyl)-, —N(C₁-C₆ alkyl)C(O)N(C₁-C₆ alkyl)-, —C(O)N(H)—, —C(O)N(C₁-C₆ alkyl)-, —C(O)N(C₃-C₈ cycloalkyl)-, arylene, heteroarylene, heterocyclylene, C₃-C₈ cycloalkylene, or C₃-C₈ cycloalkenylene, wherein the alkylene, alkenylene, alkynylene, alkyl, arylene, heteroarylene, and heterocyclylene are each optionally and independently substituted with 1, 2, or 3 R^{Z}, wherein each R^{Z} is independently OH, C₁₋₆ alkyl, or halogen;
each R¹ is independently C₁₋₆ alkyl, C₃-C₈ cycloalkyl, or halogen;
Q is absent, C₁₋₆ alkylene, —C₁₋₆ alkylene-C(O)—, C₃₋₈ cycloalkylene, —C₃₋₈ cycloalkylene-C(O)—, —C(O)—, or —S(O)₂—;
R³ is carbocycle, heterocycle, aryl, heteroaryl, or —NR^{X}R^{Y}, wherein the carbocycle, heterocycle, aryl, and heteroaryl are each optionally substituted with 1, 2, or 3 R⁴;
each R⁴ is independently halogen, —OH, C₁₋₆ alkyl, C₁₋₆ alkoxy, —C(O)—NR^{X}R^{Y}, or —C(O)O—(C₁₋₆ alkyl);
R^{X} is H, C₁₋₆ alkyl, C₁₋₆ haloalkyl, or C₁₋₆ hydroxyalkyl;
R^{Y} is C₁₋₆ alkyl, C₃₋₈ cycloalkyl, C₃₋₈ hydroxycycloalkyl, aryl, or heterocycle; and
a is an integer of 0-3.

### L²

In embodiments of the compound of formula (X-III), (III), (III-A), (III-B), (III-C), (III-D), (III-A-1), (III-A-2), (III-A-3), (III-A-4), (III-B3-1), (III-B-2), (III-B-3), (III-B-4), (III-C-1), (III-C-2), (III-C-3), (III-C-4), (III-D-1), (III-D-2), (III-D-3), or (III-D-4), or a pharmaceutically acceptable salt, stereoisomer, or deuterated form thereof, L² is C₂-C₅₀ alkylene, C₂-C₂₅ alkenylene, C₂-C₂₅ alkynylene, or —(C₂-C₅₀ alkylene)-arylene, wherein 1-25 methylene groups of L² are optionally and independently replaced by —N(H)—, —N(C₁-C₆ alkyl)-, —N(C₃-C₈ cycloalkyl)-, —O—, —C(O)—, —C(O)O—, —S—, —S(O)—, —S(O)₂—, —S(O)₂N(C₁-C₆ alkyl)-, —S(O)₂N(C₃-C₈ cycloalkyl)-, —N(H)C(O)—, —N(C₁-C₆ alkyl)C(O)—, —N(C₃-C₈ cycloalkyl)C(O)—, —N(H)C(O)N(H)—, —N(C₁-C₆ alkyl)C(O)N(H)—, —N(H)C(O)N(C₁-C₆ alkyl)-, —N(C₁-C₆ alkyl)C(O)N(C₁-C₆ alkyl)-, —C(O)N(H)—, —C(O)N(C₁-C₆ alkyl)-, —C(O)N(C₃-C₈ cycloalkyl)-, arylene, heteroarylene, heterocyclylene, C₃-C₈ cycloalkylene, or C₃-C₈ cycloalkenylene, wherein the alkylene, alkenylene, alkynylene, alkyl, arylene, heteroarylene, and heterocyclylene are each optionally and independently substituted. In embodiments, the alkylene, alkenylene, alkynylene, alkyl, arylene, heteroarylene, and heterocyclylene are each optionally and independently substituted with 1, 2, or 3 R^{Z}, wherein each R^{Z} is independently OH, C₁₋₆ alkyl, or halogen.

In embodiments of the compound of formula (X-III), (III), (III-A), (III-B), (III-C), (III-D), (III-A-1), (III-A-2), (III-A-3), (III-A-4), (III-B3-1), (III-B-2), (III-B-3), (III-B-4), (III-C-1), (III-C-2), (III-C-3), (III-C-4), (III-D-1), (III-D-2), (III-D-3), or (III-D-4), or a pharmaceutically acceptable salt, stereoisomer, or deuterated form thereof, 1-25 methylene groups of L² are optionally and independently replaced by —N(H)—, —N(C₁-C₆ alkyl)-, —N(C₃-C₈ cycloalkyl)-, —O—, —C(O)—, —C(O)O—, —N(H)C(O)—, —N(C₁-C₆ alkyl)C(O)—, —N(C₃-C₈ cycloalkyl)C(O)—, —C(O)N(H)—, —C(O)N(C₁-C₆ alkyl)-, —C(O)N(C₃-C₈ cycloalkyl)-, arylene, heteroarylene, heterocyclylene, or C₃-C₈ cycloalkylene, wherein each arylene or heteroarylene is optionally and independently substituted. In some embodiments, each arylene or heteroarylene is optionally and independently substituted with 1 or 2 R^{Z}, wherein each R^{Z} is independently OH, C₁₋₆ alkyl, or halogen.

In embodiments of the compound of formula (X-III), (III), (III-A), (III-B), (III-C), (III-D), (III-A-1), (III-A-2), (III-A-3), (III-A-4), (III-B3-1), (III-B-2), (III-B-3), (III-B-4), (III-C-1), (III-C-2), (III-C-3), (III-C-4), (III-D-1), (III-D-2), (III-D-3), or (III-D-4), or a pharmaceutically acceptable salt, a stereoisomer, or a deuterated form thereof, wherein L² is:
—(C₁₋₆ alkylene)-NH(CH₂CH₂O)ₘ—(CH₂)ₙ—NH—,
—(C₁₋₆ alkylene)-NH(CH₂CH₂O)ₘ—(CH₂)ₙ—N(C₁₋₆ alkyl)-,
—(C₁₋₆ alkylene)-NH(CH₂CH₂O)ₘ—(CH₂)ₙ—,
—(C₁₋₆ alkylene)-NH(CH₂CH₂O)ₘ—(CH₂)ₙ—NH—C(O)—(C₁₋₆ alkylene)-O—, —(C₁₋₆ alkylene)-NH(CH₂CH₂O)ₘ—(CH₂)—HET^{B}-, or
—(C₁₋₆ alkylene)-N(H)(CH₂CH₂O)ₘ—(CH₂)ₙ—HET^{B}-(C₁₋₆ alkylene)-,
wherein each alkylene or alkyl is optionally and independently substituted with 1, 2, or 3 R^{Z}, wherein each R^{Z} is independently OH, C₁₋₆ alkyl, or halogen;
HET^{B} is a 4-10 membered heterocyclylene containing 1-3 N, and is optionally substituted with —C(O)—(C₁₋₆ alkyl), —C(O)O—(C₁₋₆ alkyl), —(C₁₋₆ alkyl)-O—C₆₋₁₀ aryl, or C₁₋₆ alkyl;
m is an integer of 1-12; and
n is an integer of 0-6.

In some embodiments of the compound of formula (X-III), (III), (III-A), (III-B), (III-C), (III-D), (III-A-1), (III-A-2), (III-A-3), (III-A-4), (III-B3-1), (III-B-2), (III-B-3), (III-B-4), (III-C-1), (III-C-2), (III-C-3), (III-C-4), (III-D-1), (III-D-2), (III-D-3), or (III-D-4), or a pharmaceutically acceptable salt, a stereoisomer, or a deuterated form thereof, L² is:
—(CH₂)—NH(CH₂CH₂O)—(CH₂)₂—NH—,
—(CH₂)—NH(CH₂CH₂O)₂—(CH₂)₂—NH—,
—(CH₂)—NH(CH₂CH₂O)₃—(CH₂)₂—NH—,
—(CH₂)—NH(CH₂CH₂O)₃—(CH₂)₂—NCH₃—,
—(CH₂)—NH(CH₂CH₂O)₃—(CH₂)₃—NCH₃—,
—(CH₂)—NH(CH₂CH₂O)₄—(CH₂)₃—NCH₃—,
—(CH₂)—NH(CH₂CH₂O)₃—,
—(CH₂)—NH(CH₂CH₂O)₄—,
—(CH₂)—NH(CH₂CH₂O)₃—(CH₂)₂—NH—C(O)CH₂—O—,
—(CH₂)—NH(CH₂CH₂O)₃—(CH₂)₂—,
—(CH₂)—NH(CH₂CH₂O)₃—(CH₂)₃—,
—(CH₂)—NH(CH₂CH₂O)₄—(CH₂)₃—,

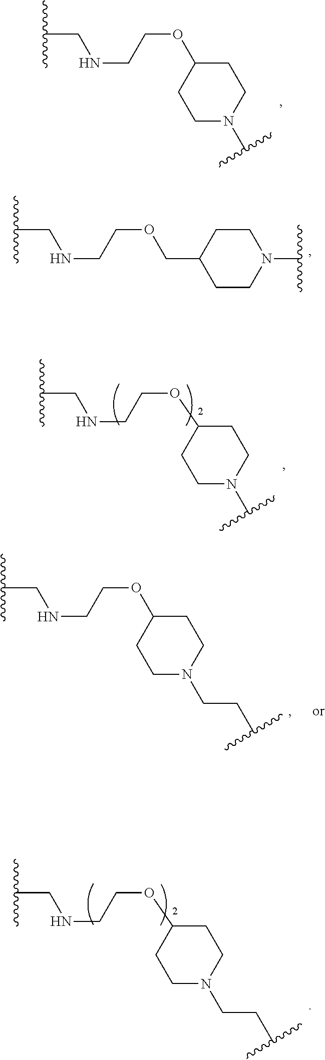

In some embodiments of the compound of formula (X-III), (III), (III-A), (III-B), (III-C), (III-D), (III-A-1), (III-A-2), (III-A-3), (III-A-4), (III-B3-1), (III-B-2), (III-B-3), (III-B-4), (III-C-1), (III-C-2), (III-C-3), (III-C-4), (III-D-1), (III-D-2), (III-D-3), or (III-D-4), or a pharmaceutically acceptable salt, a stereoisomer, or a deuterated form thereof, L² is:
—(CH₂)—NH(CH₂CH₂O)—(CH₂)₂—NH—,
—(CH₂)—NH(CH₂CH₂O)₂—(CH₂)₂—NH—,
—(CH₂)—NH(CH₂CH₂O)₃—(CH₂)₂—NH—, or
—(CH₂)—NH(CH₂CH₂O)₃—(CH₂)₂—NH—C(O)CH₂—O—.

In some embodiments of the compound of formula (X-III), (III), (III-A), (III-B), (III-C), (III-D), (III-A-1), (III-A-2), (III-A-3), (III-A-4), (III-B3-1), (III-B-2), (III-B-3), (III-B-4), (III-C-1), (III-C-2), (III-C-3), (III-C-4), (III-D-1), (III-D-2), (III-D-3), or (III-D-4), or a pharmaceutically acceptable salt, a stereoisomer, or a deuterated form thereof, L² is:

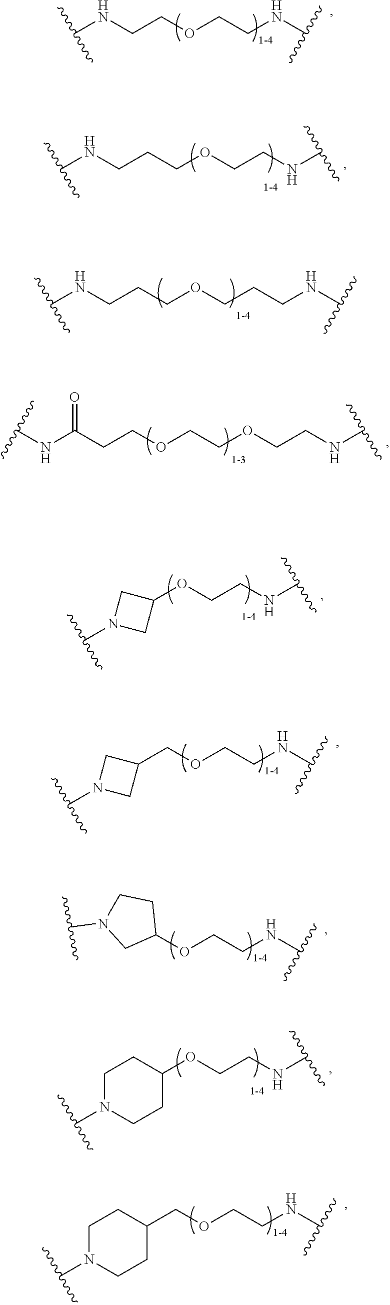

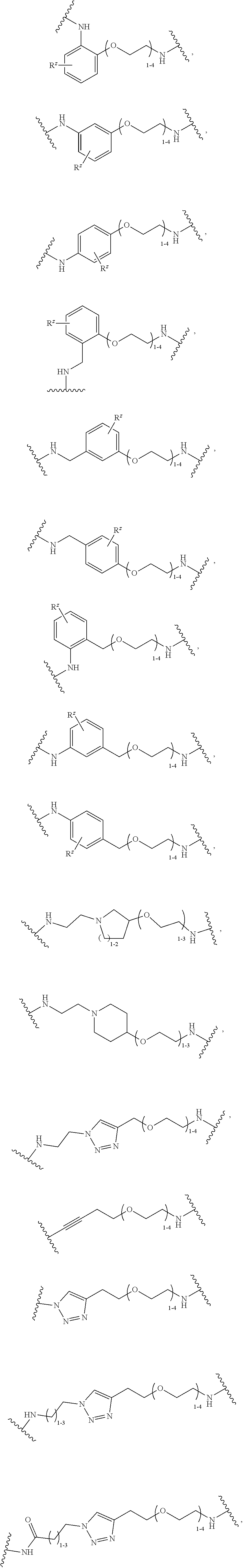

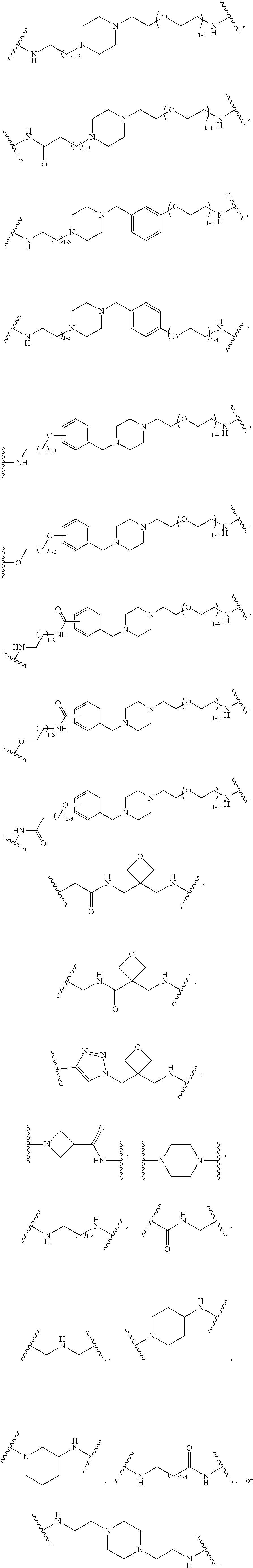

In some embodiments of the compound of formula (X-III), (III), (III-A), (III-B), (III-pharmaceutically acceptable salt, a stereoisomer, or a deuterated form thereof, L² is:

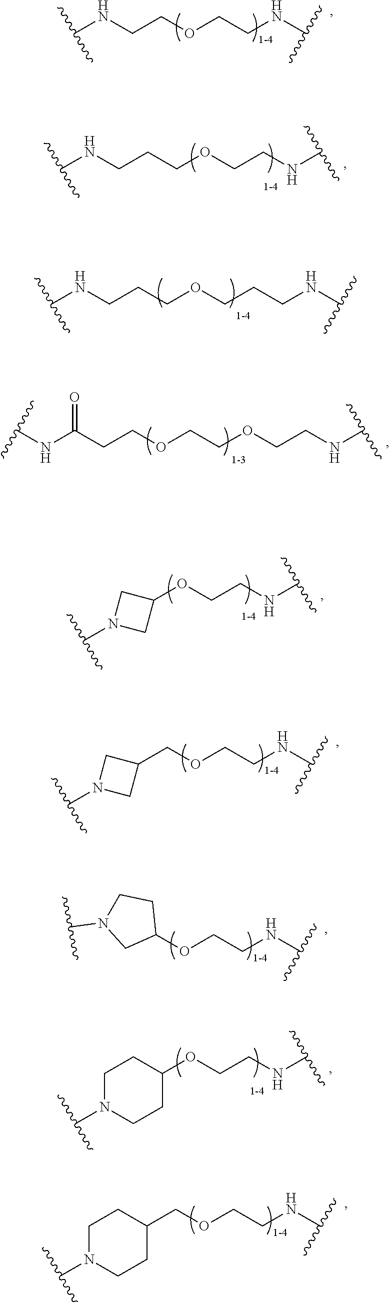

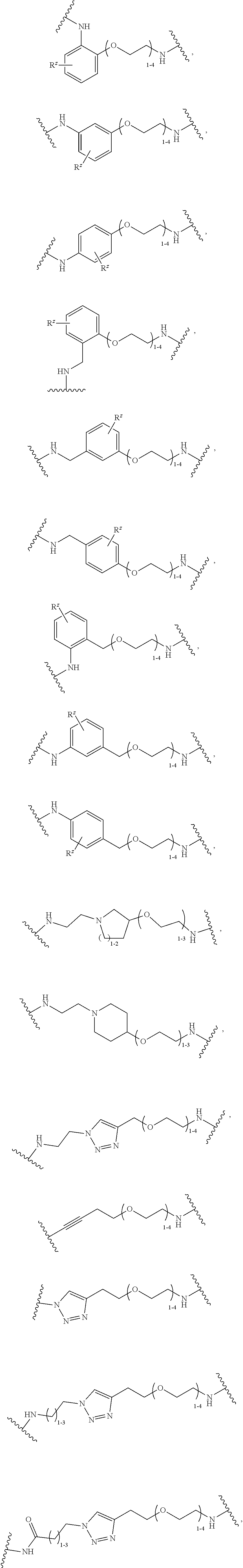

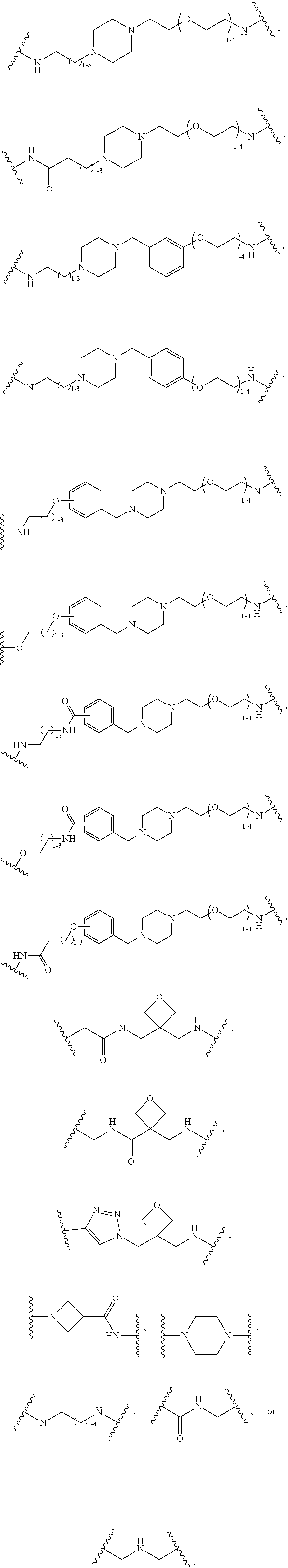

In some embodiments of the compound of formula (III), (III-A), (III-B), (III-C), (III-D), (III-A-1), (III-A-2), (III-A-3), (III-A-4), (III-B-1), (III-B-2), (III-B-3), (III-B-4), (III-C-1) (III-C-2), (III-C-3), (III-C-4), (III-D-1), (III-D-2), (III-D-3), or (III-D-4), or a pharmaceutically acceptable salt, a stereoisomer, or a deuterated form thereof, other linker moieties can be used as L² to covalently link A with ring W. Non-limiting examples of additional linker moiety as L² include the following:
(i) -Q1-(CH₂CH₂O)ₘ₁—CH₂)ₙ₁P1, -Q1CH₂CH₂CH₂O)ₘ₁—CH₂)ₙ₁P1, Q1-(CH₂CH₂NH)ₘ₁—CH₂)ₙ₁—P1-, and -Q1-(CH₂CH₂CON)ₘ₁—(CH₂)ₙ₁—P1-, wherein Q1 is —N—, —O—, ═N—, or —N(CH₃)—, which is a portion modified by binding to the moiety A; P1 is —NH—, —O—, —CH₂—, —C(═O)—; m₁ is an integer of 0 to 4; and n₁ is an integer of 0 to 3;
(ii) —(CH₂)ₙ₂—NH—C(═O)—, —(CH₂)ₙ₃—NH—C(═O)—(CH₂)ₙ₄—O—, —CH₂—CH₂—O—, —CH₂—C(═O)—, wherein n₂, n₃, and n₄ are independently an integer of 1 to 7;

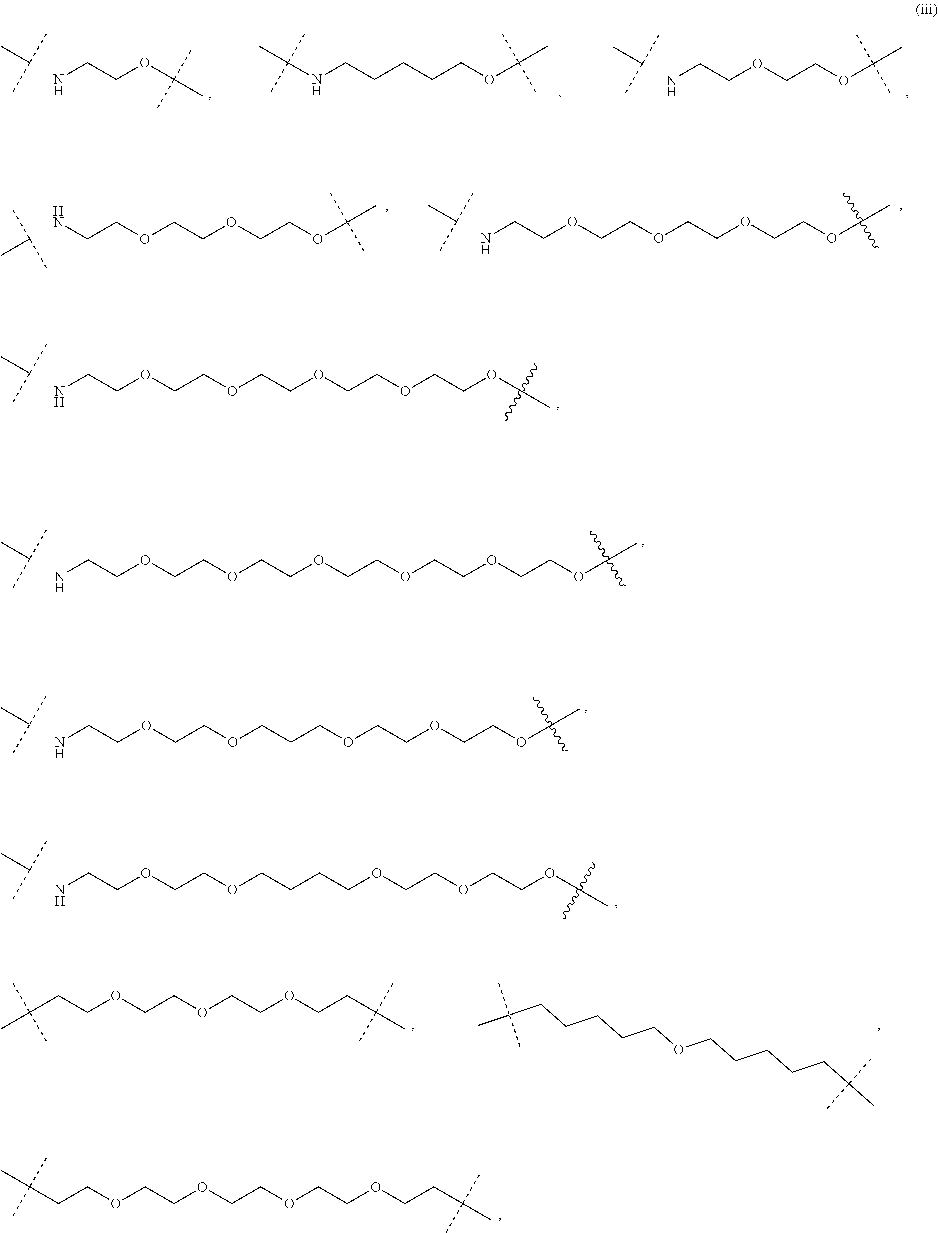

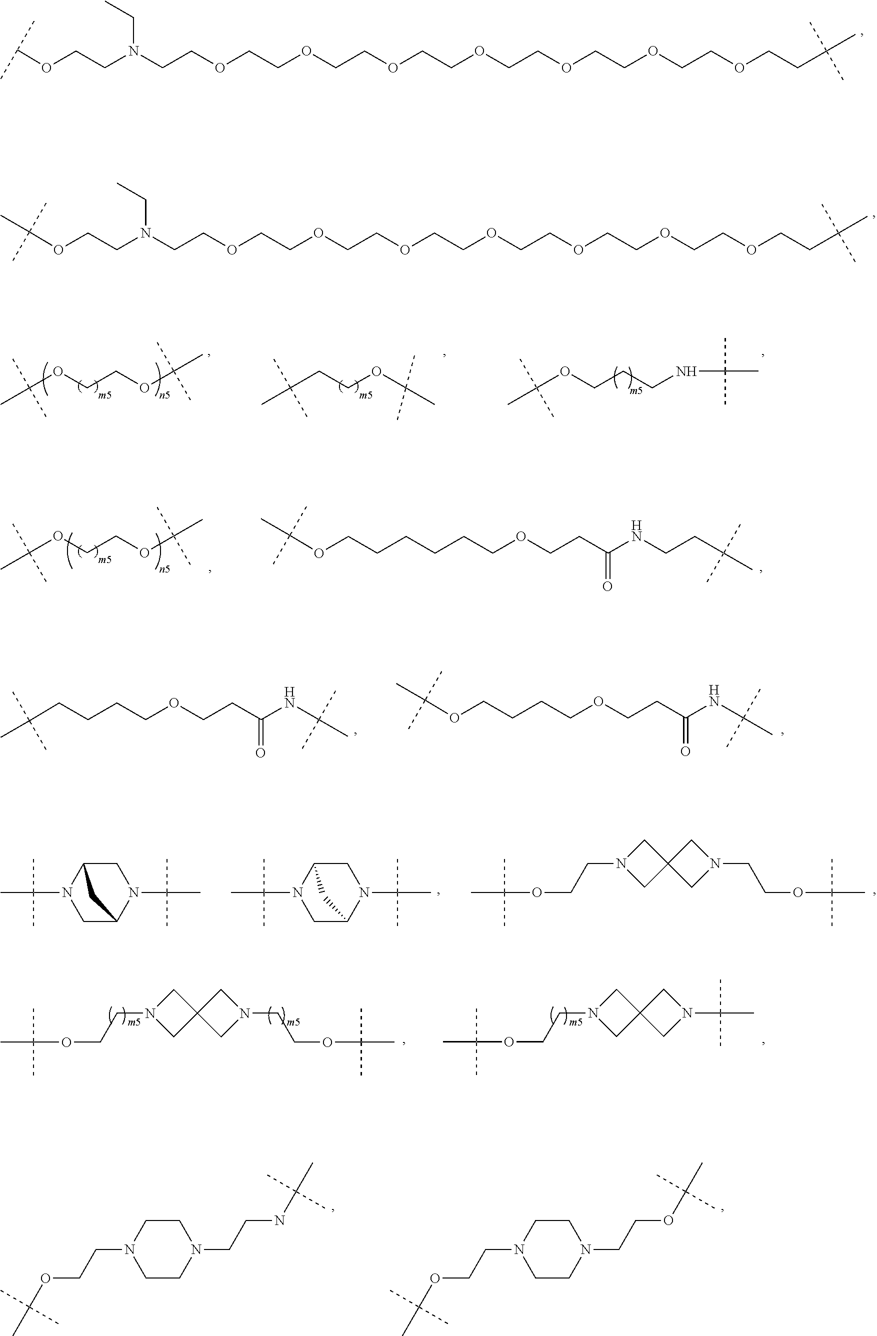

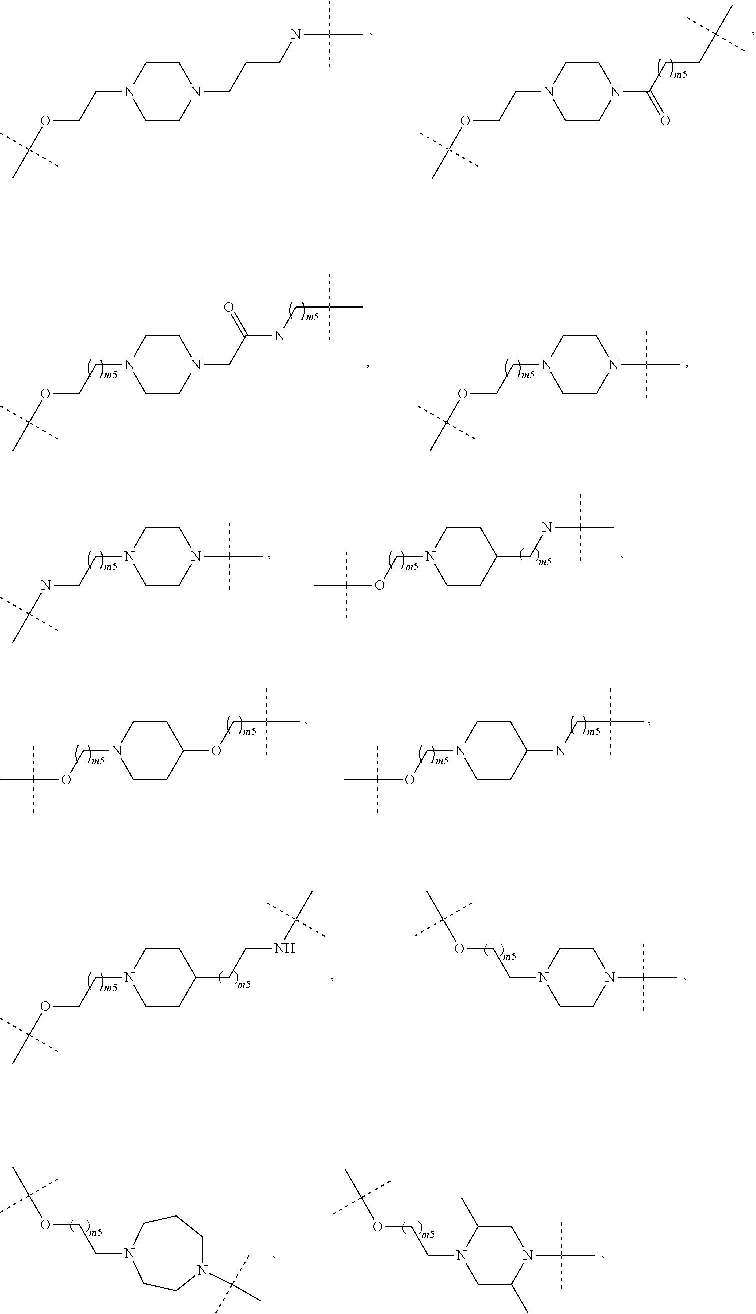

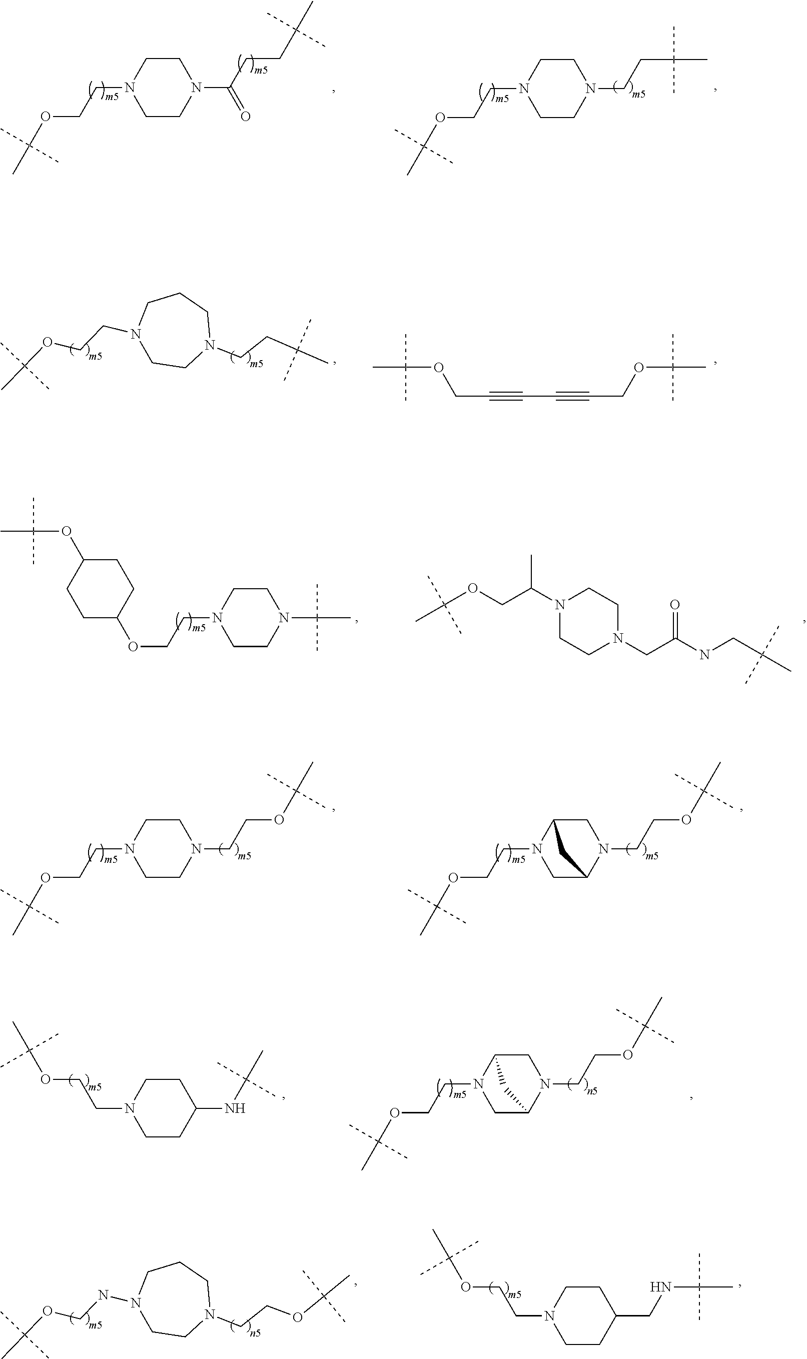

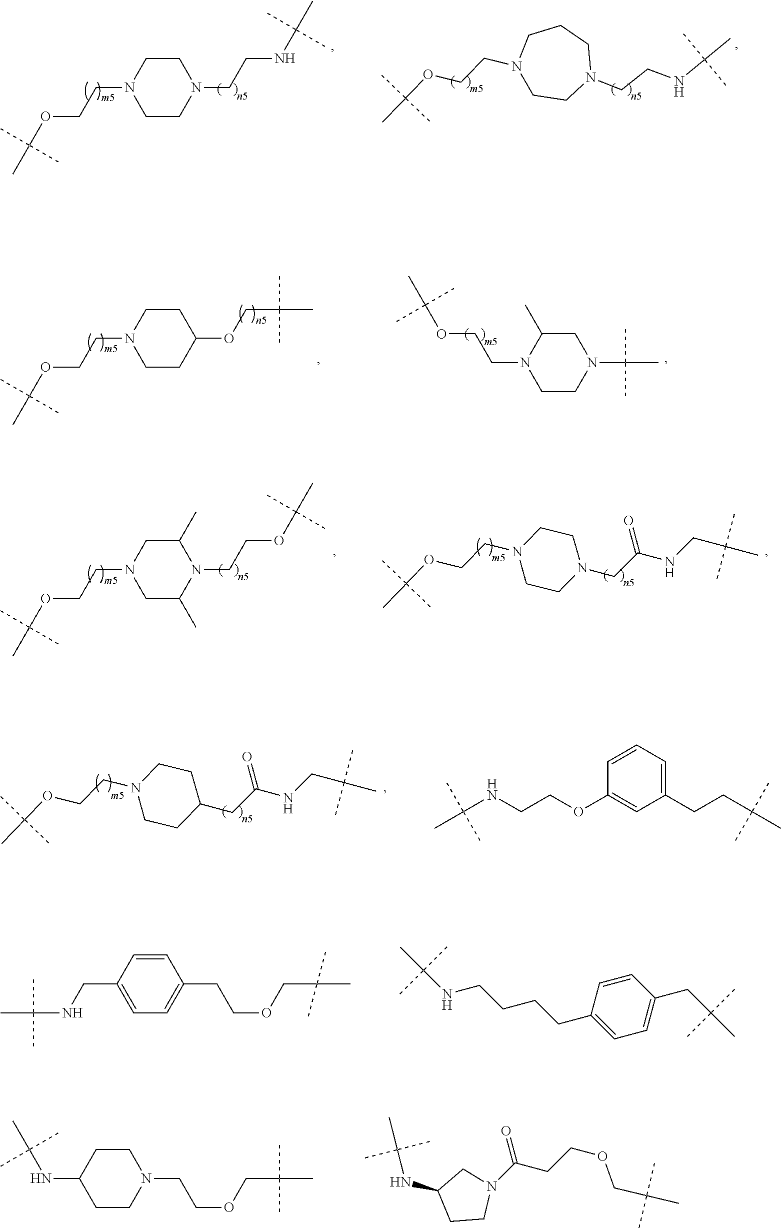

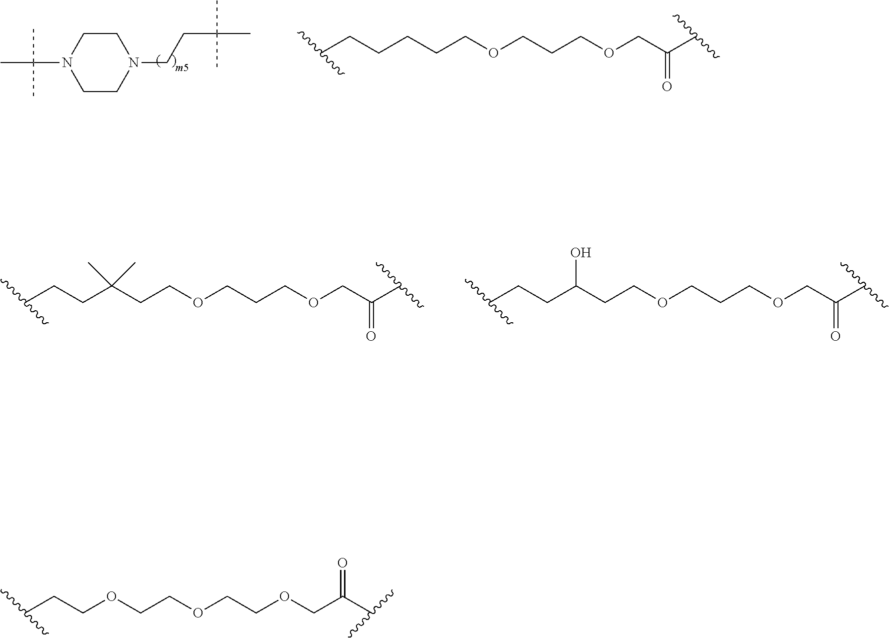

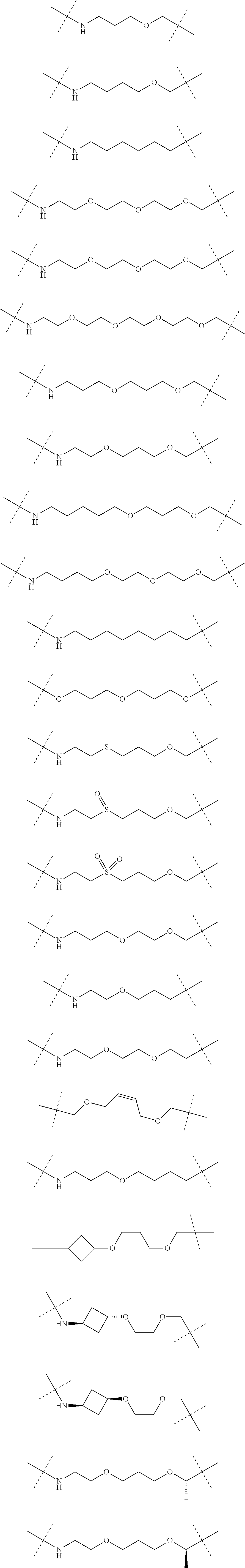

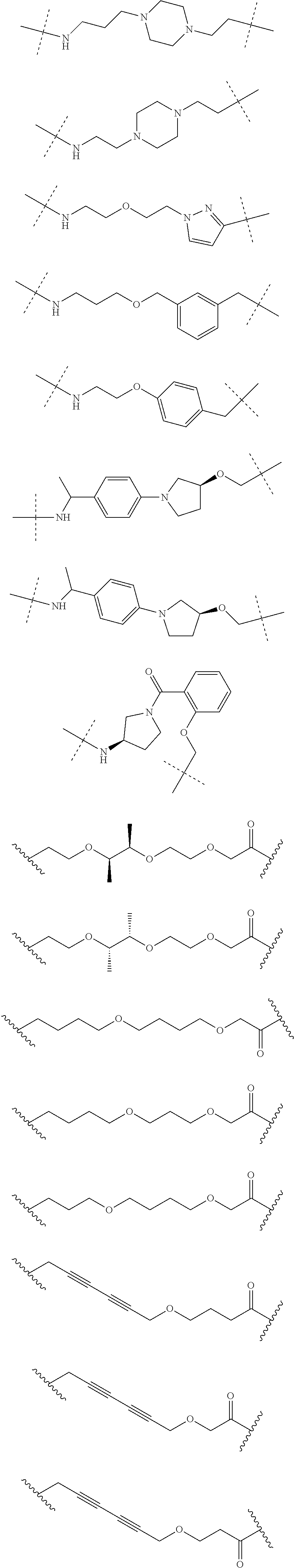

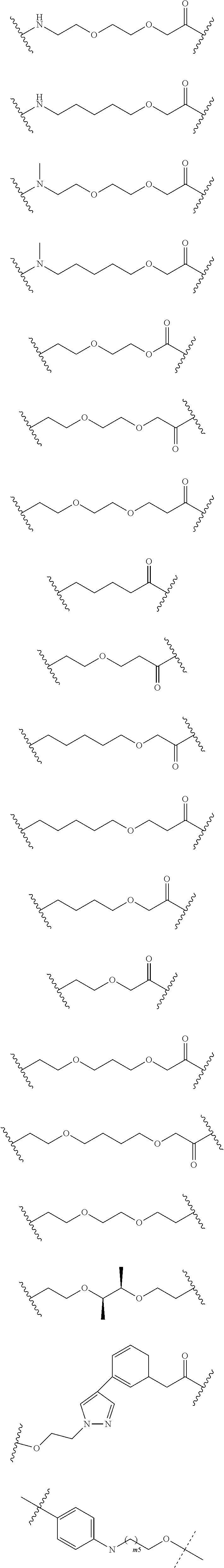

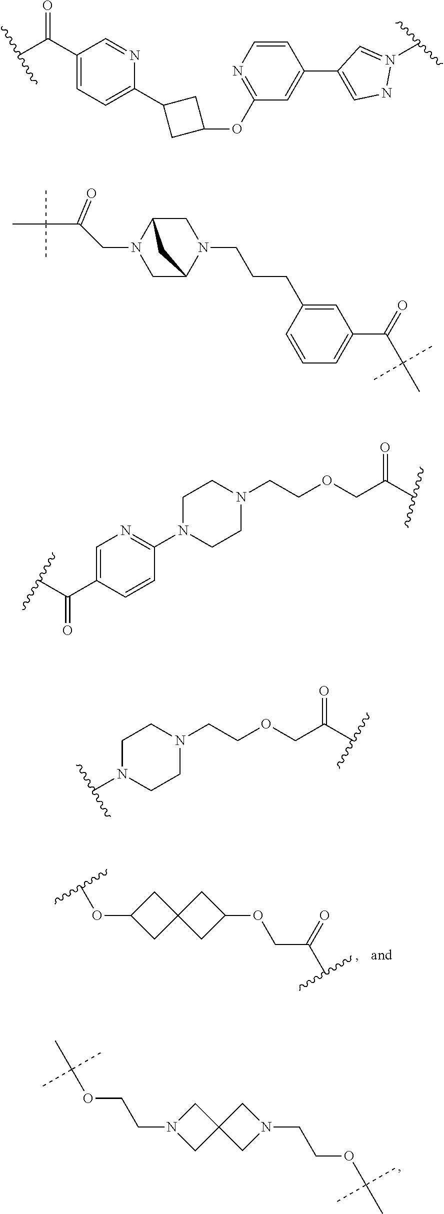

wherein m5 and n5 are each independently an integer of 0-6;
(iv) —(CH₂)ₘ₆—(OCH₂CH₂)ₙ₆O(CH₂)ₘ₆—, —O—(CH₂)ₘ₆—(OCH₂CH₂)ₙ₆O—(CH₂)ₘ₆O—, —NH—(CH₂)ₘ₆—(OCH₂CH₂)ₙ₆O—(CH₂)ₘ₆O—, —O—(CH₂)ₘ₆—(OCH₂CH₂)ₙ₆O—(CH₂)ₘ₆—NH—, —O—(CH₂)ₘ₆—NH—(CH₂)ₘ₆—(OCH₂CH₂)ₙ₆O—(CH₂)ₘ₆—, —O—(CH₂)ₘ₆—NCH₃—(CH₂)ₘ₆—(OCH₂CH₂)ₙ₆O—(CH₂)ₘ₆—, —O—(CH₂)ₘ₆—NCH₂CH₃—(CH₂)ₘ₆—(OCH₂CH₂)ₙ₆O—(CH₂)ₚ₆—, —(CH₂)ₘ₆—NH—(CH₂)ₘ₆—(OCH₂CH₂)ₙ₆O—(CH₂)ₚ₆—, —(CH₂)ₘ₆—NCH₃—(CH₂)ₘ₆—(OCH₂CH₂)ₙ₆O—(CH₂)ₚ₆—, and —(CH₂)ₘ₆—NCH₂CH₃—(CH₂)ₘ₆—(OCH₂CH₂)ₙ₆O—(CH₂)ₚ₆—,
wherein each m6 is independently an integer of 0-5, each n6 is independently an integer of 0-7, and each p6 is independently an integer of 0-2.

### Ligand A (Protein Binding Component “PBC”)

In some embodiments of the compound of formula (X-III), (III), (III-A), (III-B3), (III-C), (III-D), (III-A-1), (III-A-2), (III-A-3), (III-A-4), (III-B3-1), (III-B3-2), (III-B3-3), (III-B-4), (III-C-1), (III-C-2), (III-C-3), (III-C-4), (III-D-1), (III-D-2), (III-D-3), or (III-D-4), or a pharmaceutically acceptable salt, a stereoisomer, or a deuterated form thereof, A is a ligand (e.g., a PBC) that binds to a protein. Once a protein targeted for degradation is identified, any ligand that binds to said target protein may be used in the compounds of (X-III), (III), (III-A), (III-B), (III-C), (III-D), (III-A-1), (III-A-2), (III-A-3), (III-A-4), (III-B-1), (III-B-2), (III-B-3), (III-B-4), (III-C-1), (III-C-2), (III-C-3), (III-C-4), (III-D-1), (III-D-2), (III-D-3), or (III-D-4) described herein. Such ligands may be covalently bound to the linker using a functional group found on the ligand, or the ligand may be modified to include an appropriate functional group to facilitate conjugation to the linker.

In some embodiments of the compound of (X-III), (III), (III-A), (III-B), (III-C), (III-D), (III-A-1), (III-A-2), (III-A-3), (III-A-4), (III-B3-1), (III-B-2), (III-B-3), (III-B-4), (III-C-1), (III-C-2), (III-C-3), (III-C-4), (III-D-1), (III-D-2), (III-D-3), or (III-D-4), or a pharmaceutically acceptable salt, a stereoisomer, or a deuterated form thereof, the protein is a protein that is associated with cancer. In some embodiments, the protein associated with cancer comprises a mutation or a fusion. In some embodiments, the protein associated with cancer is BRD4. In some embodiments, the protein associated with cancer is BRD4, and A is

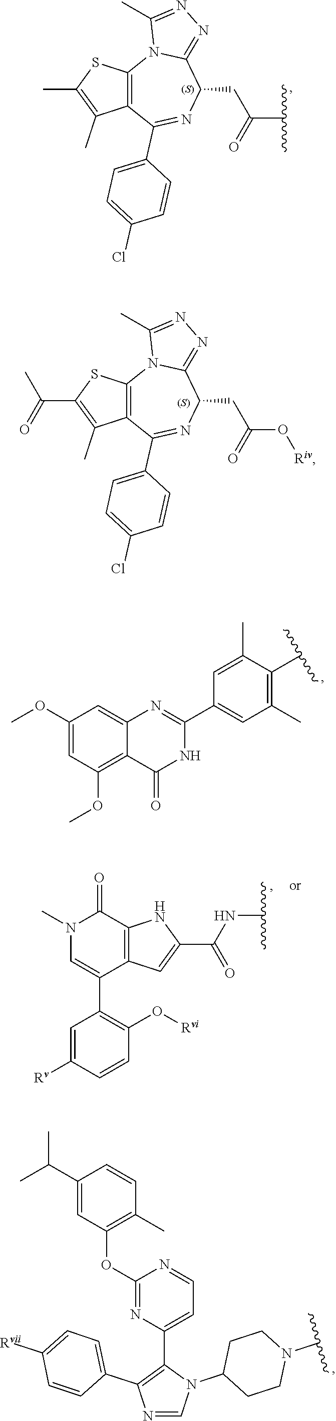

wherein:
R^{iv} is C₁₋₆ alkyl; R^{v} is H,

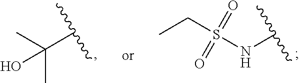

R^{vi} is

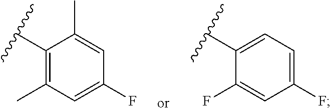

and
R^{vii} is H, F, CF₃, or C₁₋₆ alkyl.

In some embodiments, R^{iv} is methyl.

Assays for assessing the binding of BRD4 with compounds having a corresponding ligand (e.g., the compound of formula (III), (III-A), (III-B), (III-C), (III-D), (III-A-1), (III-A-2), (III-A-3), (III-A-4), (III-B3-1), (III-B-2), (III-B-3), (III-B-4), (III-C-1), (III-C-2), (III-C-3), (III-C-4), (III-D-1), (III-D-2), (III-D-3), or (III-D-4), or a pharmaceutically acceptable salt, a stereoisomer, or a deuterated form thereof having the aforementioned A group) are generally known to a person of ordinary skill in the art. Exemplary BRD4 binding assays include, but are not limited to, surface plasmon resonance (SPR) assay, fluorescence polarization (FP), and thermal shift assay (TSA). See e.g., ACS Chem. Biol. 2019, 14, 3, 361-368; J Chem Inf Model. 2023, 63(17), 5408-5432; Structure 2023, 31(8), 912-923; SLAS Discovery 2015, 20(2), 180-189; Current Protocols in Pharmacology 2018, 80, 3.16.1-3.16.14, which are incorporated by reference herein in their entirety.

In some embodiments of the compound of formula (III), (III-A), (III-B), (III-C), (III-D), (III-A-1), (III-A-2), (III-A-3), (III-A-4), (III-B3-1), (III-B-2), (III-B-3), (III-B-4), (III-C-1), (III-C-2), (III-C-3), (III-C-4), (III-D-1), (III-D-2), (III-D-3), or (III-D-4), or a pharmaceutically acceptable salt, a stereoisomer, or a deuterated form thereof, the protein is a protein that is associated with a metabolic disease.

In some embodiments of the compound of formula (III), (III-A), (III-B), (III-C), (III-D), (III-A-1), (III-A-2), (III-A-3), (III-A-4), (III-B3-1), (III-B-2), (III-B-3), (III-B-4), (III-C-1), (III-C-2), (III-C-3), (III-C-4), (III-D-1), (III-D-2), (III-D-3), or (III-D-4), or a pharmaceutically acceptable salt, a stereoisomer, or a deuterated form thereof, the protein is a protein that is associated with inflammation.

In some embodiments of the compound of formula (III), (III-A), (III-B), (III-C), (III-D), (III-A-1), (III-A-2), (III-A-3), (III-A-4), (III-B3-1), (III-B-2), (III-B-3), (III-B-4), (III-C-1), (III-C-2), (III-C-3), (III-C-4), (III-D-1), (III-D-2), (III-D-3), or (III-D-4), or a pharmaceutically acceptable salt, a stereoisomer, or a deuterated form thereof, the protein is present in bacteria.

In some embodiments of the compound of formula (III), (III-A), (III-B), (III-C), (III-D), (III-A-1), (III-A-2), (III-A-3), (III-A-4), (III-B3-1), (III-B-2), (III-B-3), (III-B-4), (III-C-1), (III-C-2), (III-C-3), (III-C-4), (III-D-1), (III-D-2), (III-D-3), or (III-D-4), or a pharmaceutically acceptable salt, a stereoisomer, or a deuterated form thereof, the protein is present in a virus particle.

In some embodiments of the compound of formula (III), (III-A), (III-B), (III-C), (III-D), (III-A-1), (III-A-2), (III-A-3), (III-A-4), (III-B3-1), (III-B-2), (III-B-3), (III-B-4), (III-C-1), (III-C-2), (III-C-3), (III-C-4), (III-D-1), (III-D-2), (III-D-3), or (III-D-4), or a pharmaceutically acceptable salt, a stereoisomer, or a deuterated form thereof, A is a ligand that binds to a protein aggregate. In some embodiments of the compound of formula (III), (III-A), (III-B), (III-C), (III-D), (III-A-1), (III-A-2), (III-A-3), (III-A-4), (III-B-1), (III-B-2), (III-B-3), (III-B-4), (III-C-1), (III-C-2), (III-C-3), (III-C-4), (III-D-1), (III-D-2), (III-D-3), or (III-D-4), or a pharmaceutically acceptable salt, a stereoisomer, or a deuterated form thereof, the protein aggregate is a Tau protein aggregate, an alpha-synuclein protein aggregate, a mutant Huntingtin protein aggregate, a β-sheet aggregate, a mitochondrial protein aggregate, an amyloid protein aggregate, or a TDP-43 protein aggregate. In some embodiments of the compound of formula (III), (III-A), (III-B), (III-C), (III-D), (III-A-1), (III-A-2), (III-A-3), (III-A-4), (III-B3-1), (III-B-2), (III-B-3), (III-B-4), (III-C-1), (III-C-2), (III-C-3), (III-C-4), (III-D-1), (III-D-2), (III-D-3), or (III-D-4), or a pharmaceutically acceptable salt, a stereoisomer, or a deuterated form thereof, the protein aggregate is an alpha-synuclein protein aggregate, a mutant Huntingtin protein aggregate, a β-sheet aggregate, an amyloid protein aggregate, or a TDP-43 protein aggregate.

In some embodiments, the protein aggregate is a Tau protein aggregate, and A is

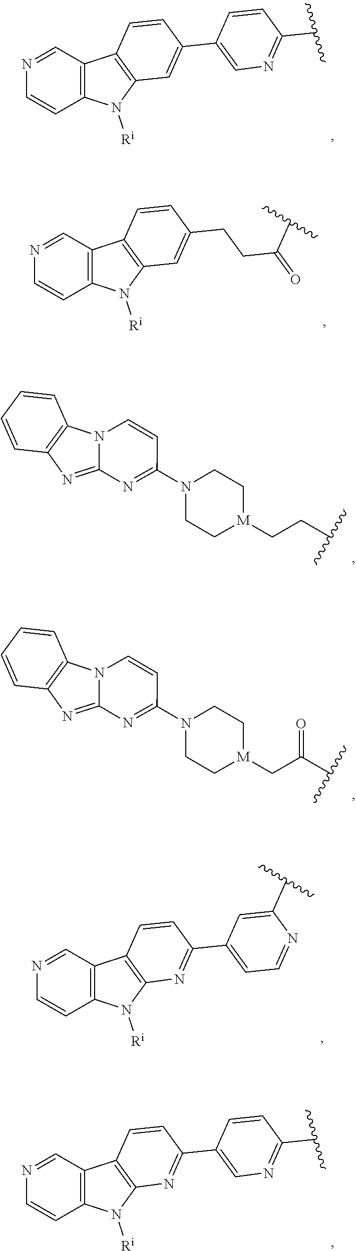

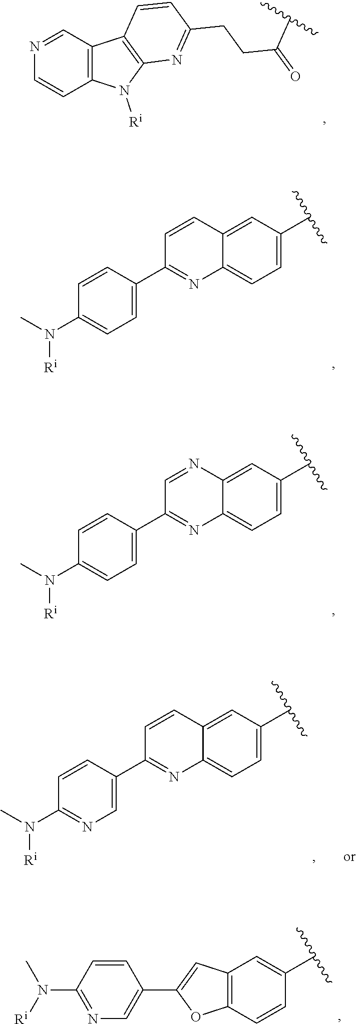

wherein: each Rⁱ is independently H or C₁₋₆ alkyl; and M is CH or N. In some embodiments, each Rⁱ is independently H or methyl.

Assays for assessing the binding of Tau with compounds having a corresponding ligand (e.g., the compound of formula (III), (III-A), (III-B), (III-C), (III-D), (III-A-1), (III-A-2), (III-A-3), (III-A-4), (III-B3-1), (III-B-2), (III-B-3), (III-B-4), (III-C-1), (III-C-2), (III-C-3), (III-C-4), (III-D-1), (III-D-2), (III-D-3), or (III-D-4), or a pharmaceutically acceptable salt, a stereoisomer, or a deuterated form thereof having the aforementioned A group) are generally known to a person of ordinary skill in the art. Exemplary Tau binding assays include, but are not limited to, radioligand binding assay, surface plasmon resonance (SPR) assay, differential scanning fluorimetry (DSF) and nuclear magnetic resonance (NMR) titration. See e.g., Chembiochem. 2023 May 16; 24(10):e202300163; J Chem Inf Model. 2023, 63(17), 5408-5432; Alzheimer's Res Therapy 2017, 9, 96; Eur J Nucl Med Mol Imaging 2024, 51, 3960-3977; bioRxiv 2024.03.15.585148, which are incorporated by reference herein in their entirety.

In some embodiments, the protein aggregate is an alpha-synuclein protein aggregate, and A is

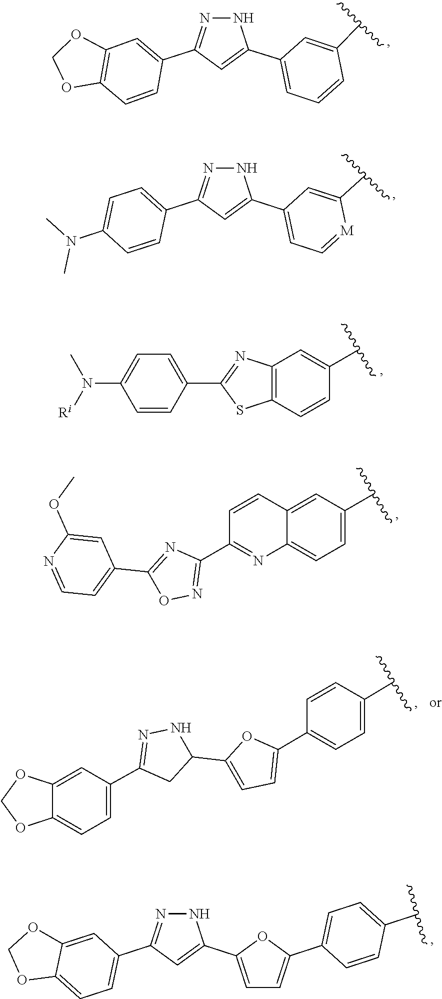

wherein: Rⁱ is H or C₁₋₆ alkyl; and M is CH or N. In some embodiments, Rⁱ is H or methyl.

Assays for assessing the binding of alpha-synuclein with compounds having a corresponding ligand (e.g., the compound of formula (III), (III-A), (III-B), (III-C), (III-D), (III-A-1), (III-A-2), (III-A-3), (III-A-4), (III-B3-1), (III-B-2), (III-B-3), (III-B-4), (III-C-1), (III-C-2), (III-C-3), (III-C-4), (III-D-1), (III-D-2), (III-D-3), or (III-D-4), or a pharmaceutically acceptable salt, a stereoisomer, or a deuterated form thereof having the aforementioned A group) are generally known to a person of ordinary skill in the art. Exemplary alpha-synuclein binding assays include, but are not limited to, nuclear magnetic resonance (NMR) spectroscopy, surface plasmon resonance (SPR) assay, and radioligand binding assay. See e.g., Chembiochem. 2023 May 16; 24(10):e202300163; J Chem Inf Model. 2023, 63(17), 5408-5432; Commun Biol 2018, 1, 44; J Neurochem. 2008, 105(4), 1428-37; Eur J Nucl Med Mol Imaging 2024, 51, 3960-3977, which are incorporated by reference herein in their entirety.

In some embodiments, the protein aggregate is a mutant Huntingtin protein aggregate (mHTT), and A is

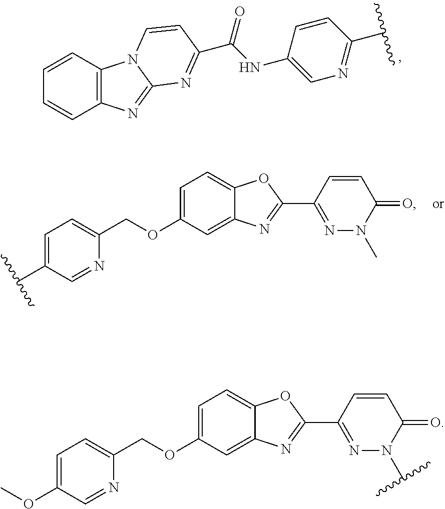 [SMILES: *c1ccc(NC(=O)c2ccn3c(n2)nc2ccccc23)cn1 ; *c1ccc(COc2ccc3oc(-c4ccc(=O)n(C)n4)nc3c2)nc1 ; *n1nc(-c2nc3cc(OCc4ccc(OC)cn4)ccc3o2)ccc1=O]

Assays for assessing the binding of mutant Huntingtin protein with compounds having a corresponding ligand (e.g., the compound of formula (III), (III-A), (III-B), (III-C), (III-D), (III-E), (III-F), (III-G), (III-A-1), (III-A-2), (III-A-3), (III-B-1), (III-C-1), (III-D-1), (III-E-1), (III-F-1), or (III-G-1), or a pharmaceutically acceptable salt, a stereoisomer, or a deuterated form thereof having the aforementioned A group) are generally known to a person of ordinary skill in the art. Exemplary mutant Huntingtin protein binding assays include, but are not limited to, surface plasmon resonance (SPR) assay, differential scanning fluorimetry (DSF), and radioligand binding assay. See e.g., Chembiochem. 2023 May 16; 24(10):e202300163; J Chem Inf Model. 2023, 63(17), 5408-5432; Structure 2023, 31(9), 1121-1131.e6; Sci Rep 2021, 11, 17977, which are incorporated by reference herein in their entirety.

In some embodiments, the protein aggregate is a p-sheet aggregate, and A is

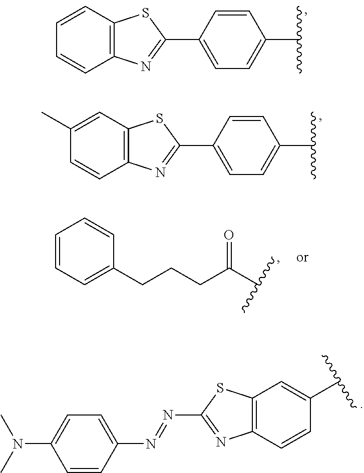

Assays for assessing the binding of p-sheet aggregate with compounds having a corresponding ligand (e.g., the compound of formula (III), (III-A), (III-B), (III-C), (III-D), (III-E), (III-F), (III-G), (III-A-1), (III-A-2), (III-A-3), (III-B3-1), (III-C-1), (III-D-1), (III-E-1), (III-F-1), or (III-G-1), or a pharmaceutically acceptable salt, a stereoisomer, or a deuterated form thereof having the aforementioned A group) are generally known to a person of ordinary skill in the art. Exemplary β-sheet aggregate binding assays include, but are not limited to, fluorescence spectroscopy, surface plasmon resonance (SPR) assay, differential scanning fluorimetry (DSF), nanoscale differential scanning fluorimetry (nanoDSF), and thermal shift assay (TSA). See e.g., Chembiochem. 2023 May 16; 24(10):e202300163; J Chem Inf Model. 2023, 63(17), 5408-5432; Anal Biochem. 2022, 654, 114828; Eur J Nucl Med Mol Imaging 2024, 51, 3960-3977, which are incorporated by reference herein in their entirety.

In some embodiments, the protein is a mitochondrial protein, and A is

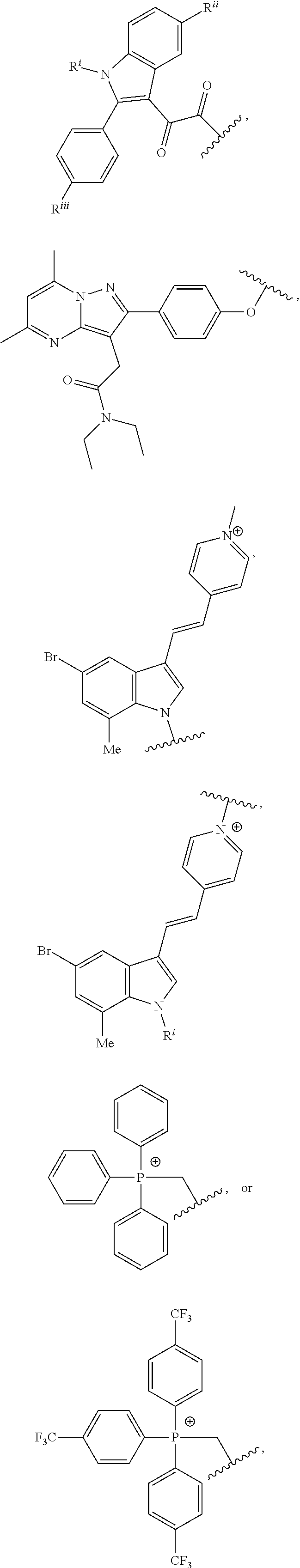

wherein: Rⁱ is H or C₁₋₆ alkyl, and Rⁱⁱ and Rⁱⁱⁱ are each independently a halogen or an alkyl. In some embodiments, Rⁱ is H or methyl, and Rⁱⁱ and Rⁱⁱⁱ are each independently F, Cl, or C₁₋₆ alkyl.

Assays for assessing the binding of mitochondrial protein with compounds having a corresponding ligand (e.g., the compound of formula (III), (III-A), (III-B), (III-C), (III-D), (III-E), (III-F), (III-G), (III-A-1), (III-A-2), (III-A-3), (III-B3-1), (III-C-1), (III-D-1), (III-E-1), (III-F-1), or (III-G-1), or a pharmaceutically acceptable salt, a stereoisomer, or a deuterated form thereof having the aforementioned A group) are generally known to a person of ordinary skill in the art. Exemplary mitochondrial protein binding assays include, but are not limited to, radioligand binding assay, surface plasmon resonance (SPR) assay, thermal shift assay (TSA), and X-ray crystallography. See e.g., Chembiochem. 2023 May 16; 24(10):e202300163; J Chem Inf Model. 2023, 63(17), 5408-5432; Journal of Med. Chem. 2004, 47(7), 1852-1855; Biochemistry 2023, 62, 7, 1262-1273, which are incorporated by reference herein in their entirety.

In some embodiments of the compound of formula (III), (III-A), (III-B), (III-C), (III-D), (III-A-1), (III-A-2), (III-A-3), (III-A-4), (III-B3-1), (III-B3-2), (III-B3-3), (III-B-4), (III-C-1), (III-C-2), (III-C-3), (III-C-4), (III-D-1), (III-D-2), (III-D-3), or (III-D-4), or a pharmaceutically acceptable salt, a stereoisomer, or a deuterated form thereof, the protein is an intracellular protein.

In some embodiments of the compound of formula (III), (III-A), (III-B), (III-C), (III-D), (III-A-1), (III-A-2), (III-A-3), (III-A-4), (III-B3-1), (III-B3-2), (III-B3-3), (III-B-4), (III-C-1), (III-C-2), (III-C-3), (III-C-4), (III-D-1), (III-D-2), (III-D-3), or (III-D-4), or a pharmaceutically acceptable salt, a stereoisomer, or a deuterated form thereof, A is aryl, arylene-heteroaryl, arylene, heteroarylene, aryl, heteroarylene-aryl, heteroarylene-heteroaryl, heteroarylene-heteroarylene-heteroaryl, heterocyclylene-aryl, heterocyclylene-heteroaryl, heteroarylene-NC(O)-heteroaryl, heteroarylene-N═N-aryl, or arylene-(arylalkyl)-OC(O)-hetercyclyl-C(O)C₁₋₆alkylene-aryl, each of which is optionally substituted with 1, 2, 3, or 4 groups independently selected from halogen, C₁₋₆ alkyl, O—C₁₋₆ alkyl, NH₂, NH(C₁₋₆ alkyl), N(C₁₋₆ alkyl)₂, —(CH₂)₁₋₄C(O)NH₂, —(CH₂)₁₋₄C(O)NH(C₁₋₆ alkyl), or —(CH₂)₁₋₄C(O)N(C₁₋₆ alkyl)₂.

In some embodiments of the compound of formula (III), (III-A), (III-B), (III-C), (III-D), (III-A-1), (III-A-2), (III-A-3), (III-A-4), (III-B3-1), (III-B3-2), (III-B3-3), (III-B-4), (III-C-1), (III-C-2), (III-C-3), (III-C-4), (III-D-1), (III-D-2), (III-D-3), or (III-D-4), or a pharmaceutically acceptable salt, a stereoisomer, or a deuterated form thereof, A is

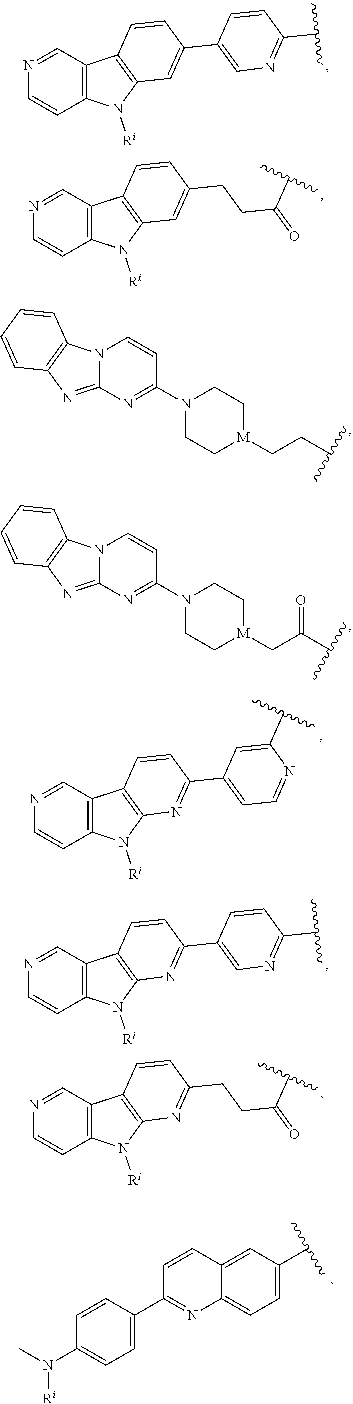

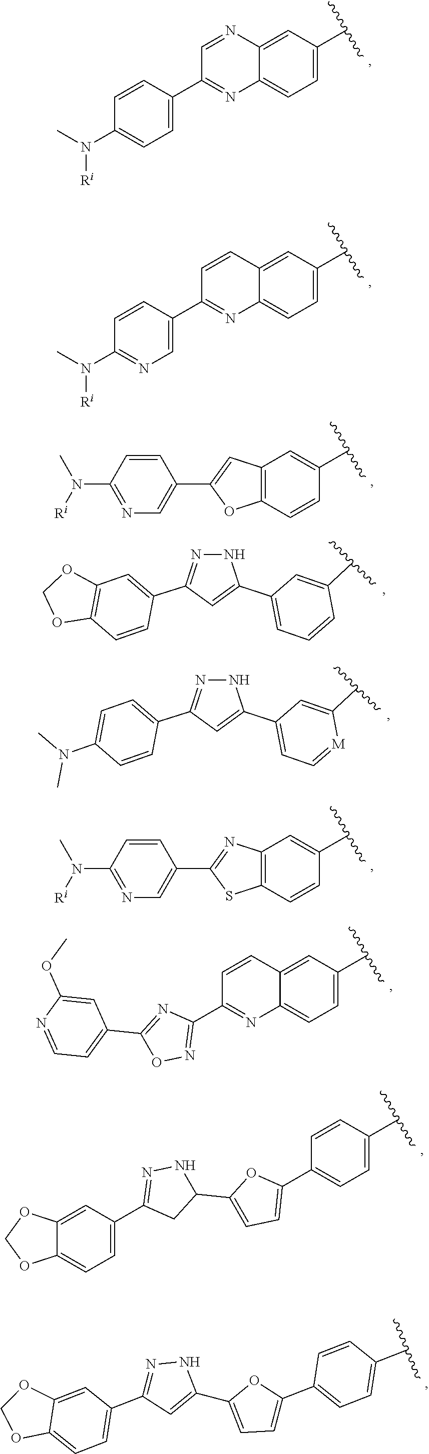

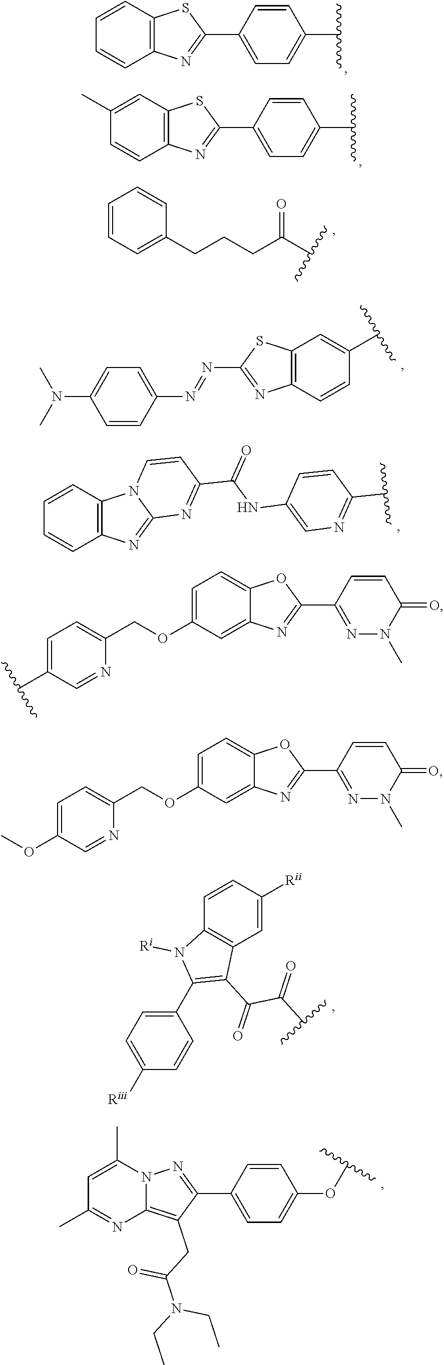

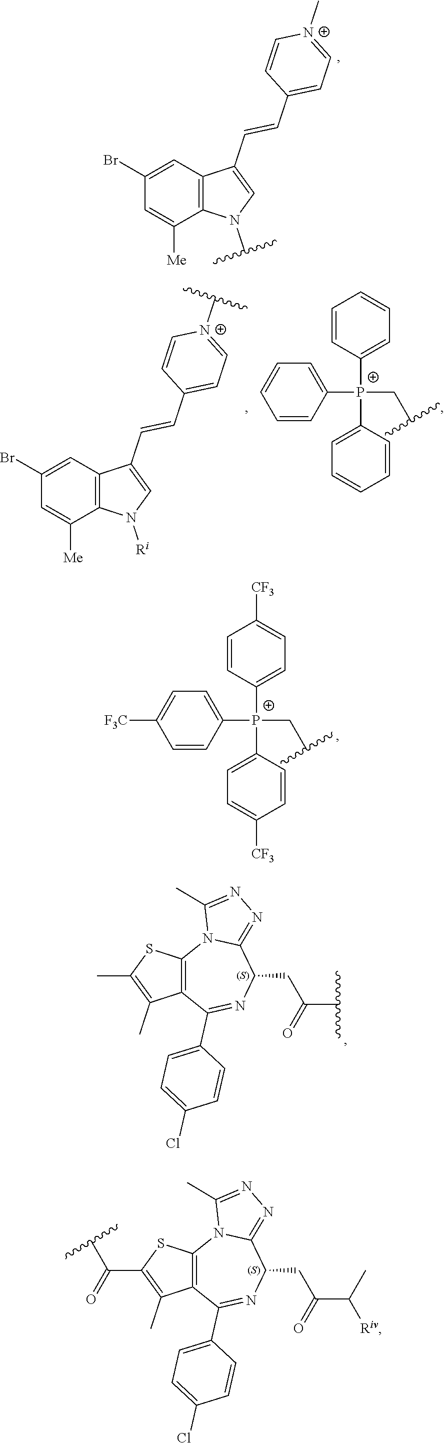

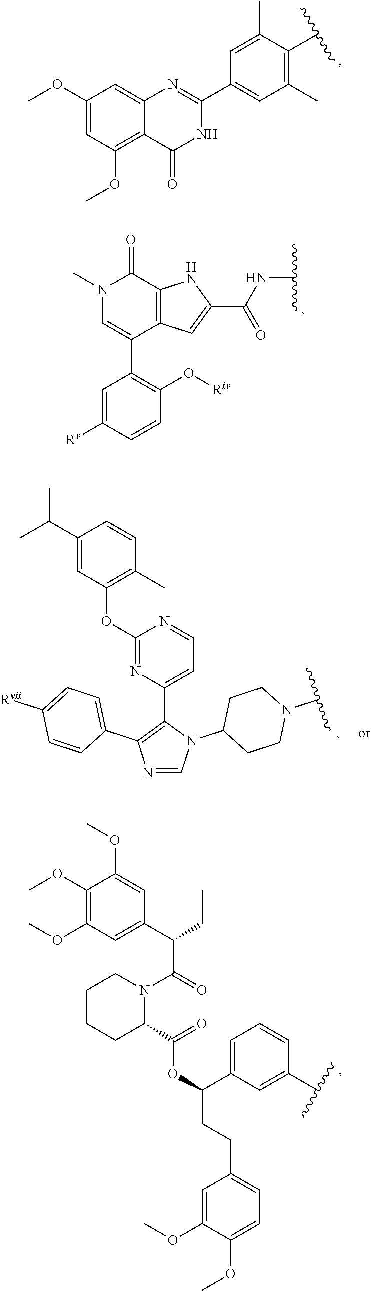

wherein:
each Rⁱ is independently H or C₁₋₆ alkyl;
Rⁱⁱ and Rⁱⁱⁱ are each independently F, Cl, or C₁₋₆ alkyl;
R^{iv} is C₁₋₆ alkyl;
R^{v} is H,

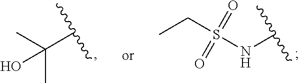

R^{vi} is

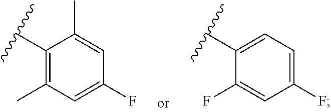

R^{vii} is H, F, CF₃, or C₁₋₆ alkyl; and
M is CH or N.

In some embodiments of the compound of formula ((III), (III-A), (III-B), (III-C), (III-D), (III-A-1), (III-A-2), (III-A-3), (III-A-4), (III-B3-1), (III-B-2), (III-B-3), (III-B-4), (III-C-1), (III-C-2), (III-C-3), (III-C-4), (III-D-1), (III-D-2), (III-D-3), or (III-D-4), or a pharmaceutically acceptable salt, a stereoisomer, or a deuterated form thereof, A is

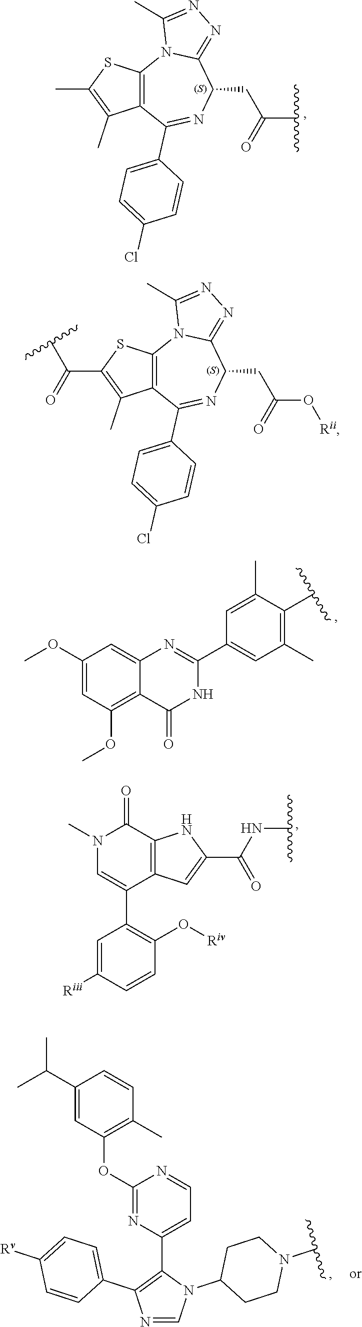

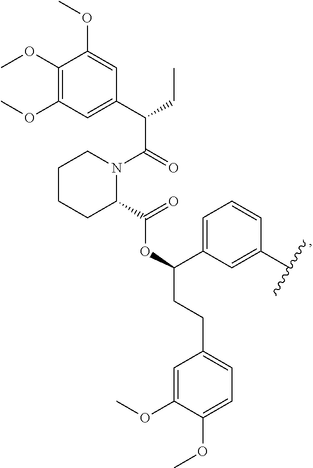

wherein:
Rⁱⁱ is C₁₋₆ alkyl;
Rⁱⁱⁱ is H,

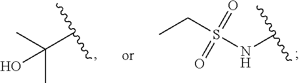

R^{iv} is

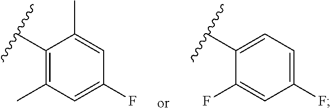

and
R^{v} is H, F, CF₃, or C₁₋₆ alkyl.

In some embodiments of the compound of formula (III), (III-A), (III-B), (III-C), (III-D), (III-A-1), (III-A-2), (III-A-3), (III-A-4), (III-B3-1), (III-B-2), (III-B-3), (III-B-4), (III-C-1), (III-C-2), (III-C-3), (III-C-4), (III-D-1), (III-D-2), (III-D-3), or (III-D-4), or a pharmaceutically acceptable salt, a stereoisomer, or a deuterated form thereof, other protein binding components (PBCs) that binds to a specific target protein, protein aggregates, protein complexes, lipids, or lipids can be used as ligand A. Non-limiting examples of the protein binding component include the following:

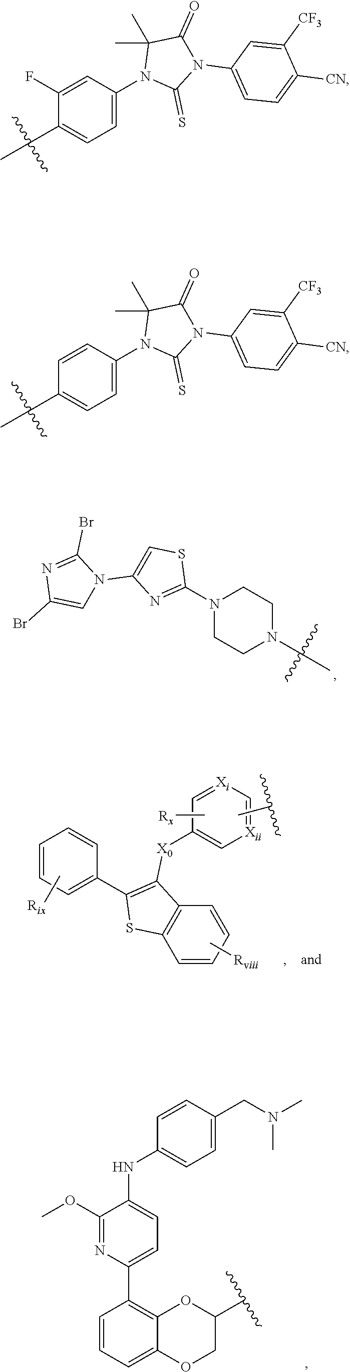

wherein:
X₀ is O or C═O;
Xᵢ and Xᵢᵢ are each independently N or CH;
Rᵥᵢᵢᵢ is OH, O(CO)Rᵢₓ, O—C₁₋₆ alkyl, wherein Rix is an alkyl or aryl group;
Rᵢₓ is H, OH, halogen, CN, CF₃, SO₂—C₁₋₆ alkyl, O—C₁₋₆ alkyl; and
Rₓ is H or halogen.

In some embodiments, the protein binding component is a derivative (e.g., a monovalent derivative that covalently bonded to linker L²) of one of the following compounds:

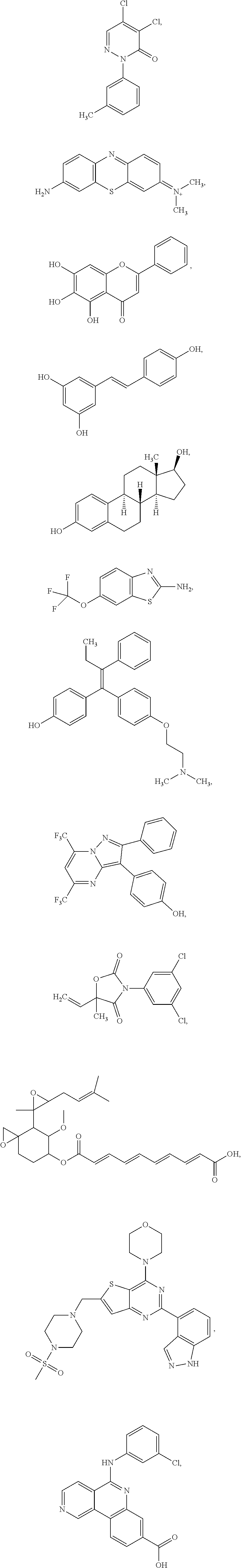

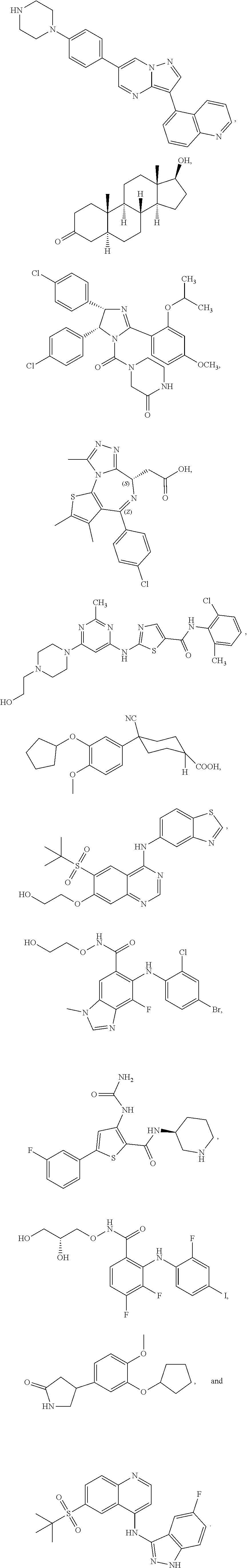

As a non-limiting example, a monovalent derivative of compound

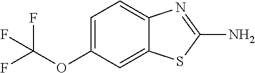 [SMILES: Nc1nc2ccc(OC(F)(F)F)cc2s1]

may be:

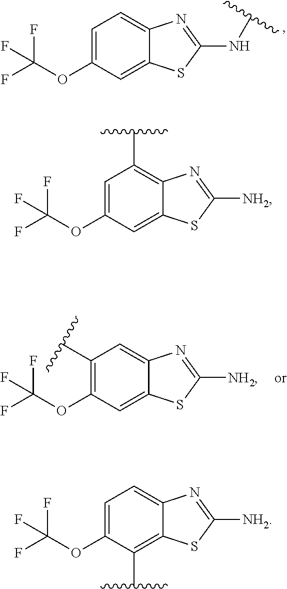 [SMILES: *Nc1nc2ccc(OC(F)(F)F)cc2s1 ; *c1cc(OC(F)(F)F)cc2sc(N)nc12 ; *c1cc2nc(N)sc2cc1OC(F)(F)F ; *c1c(OC(F)(F)F)ccc2nc(N)sc12]

In some embodiments, the protein binding component is an androgen receptor (AR) binding ligand. Non-limiting examples of androgen receptor binding ligand as protein binding component include:

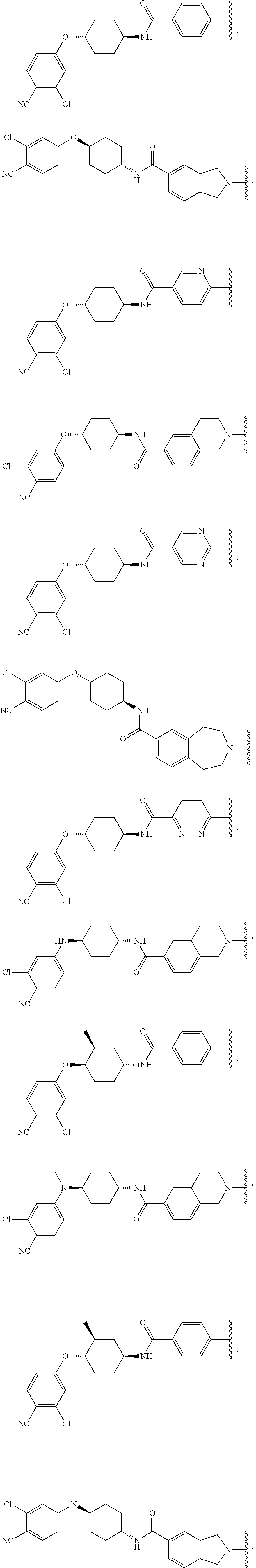

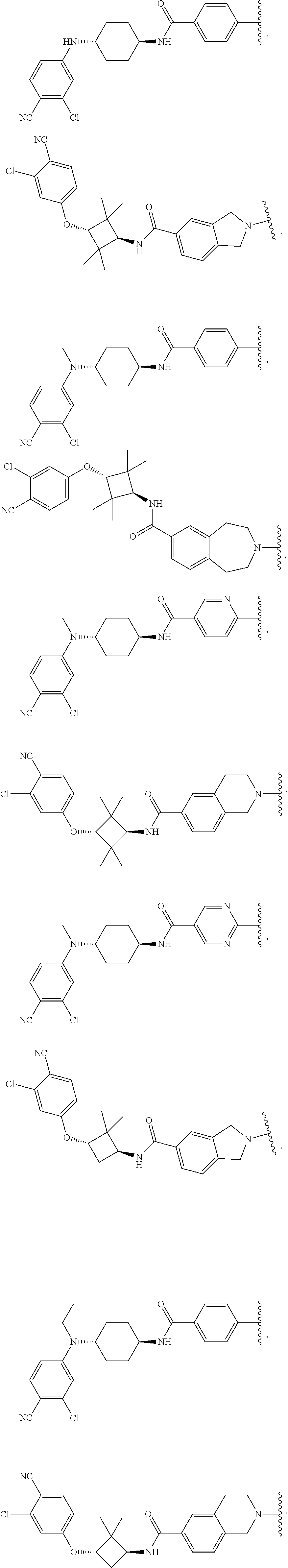

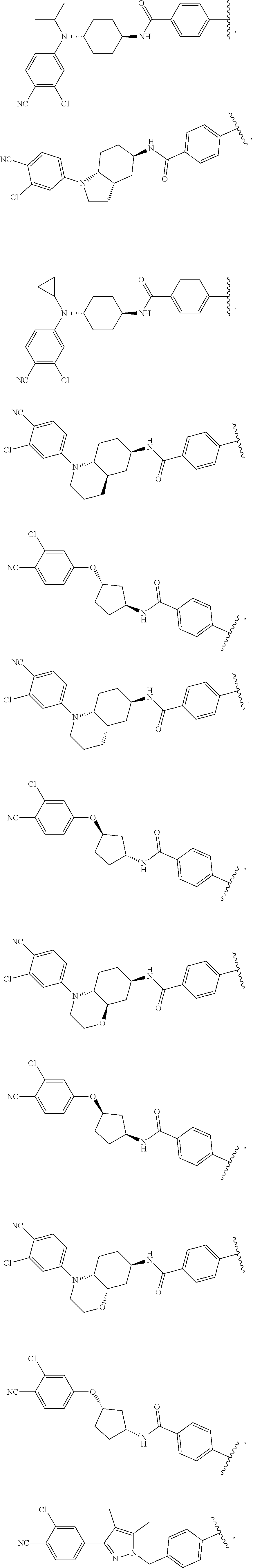

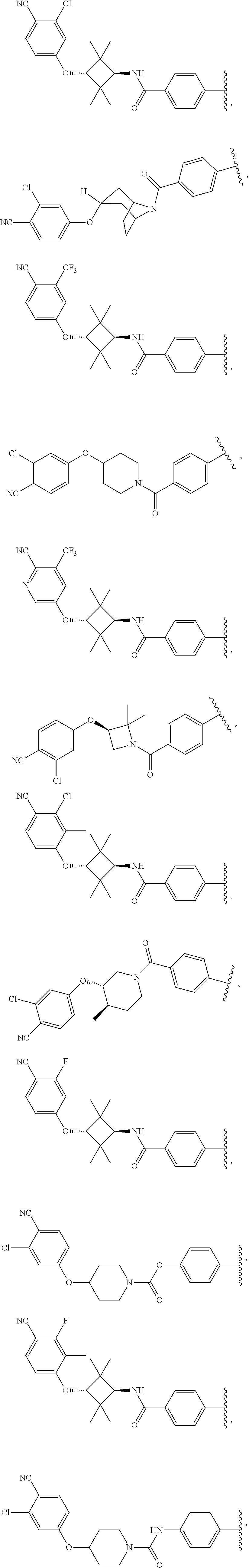

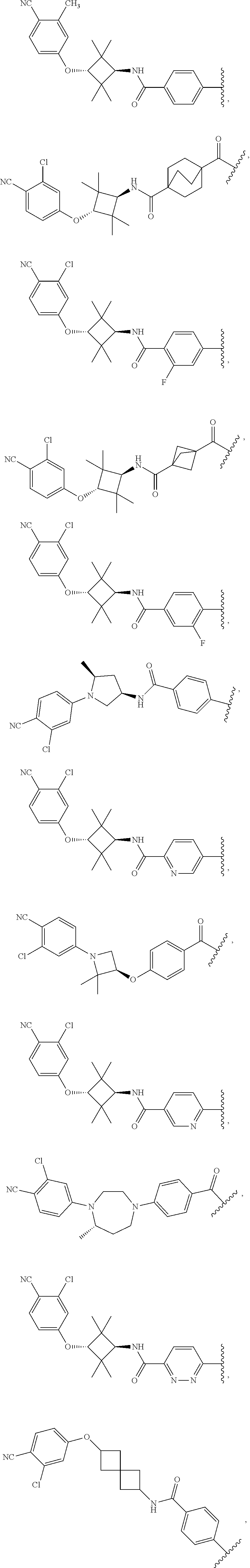

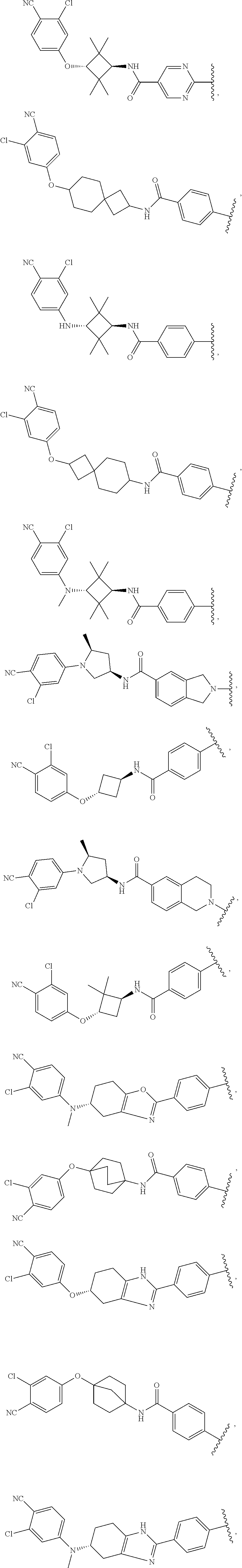

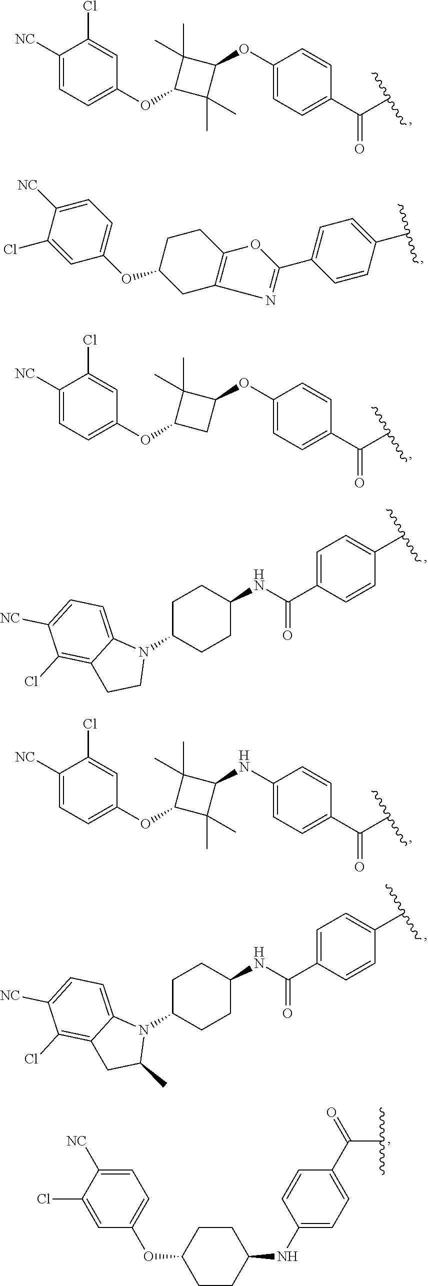

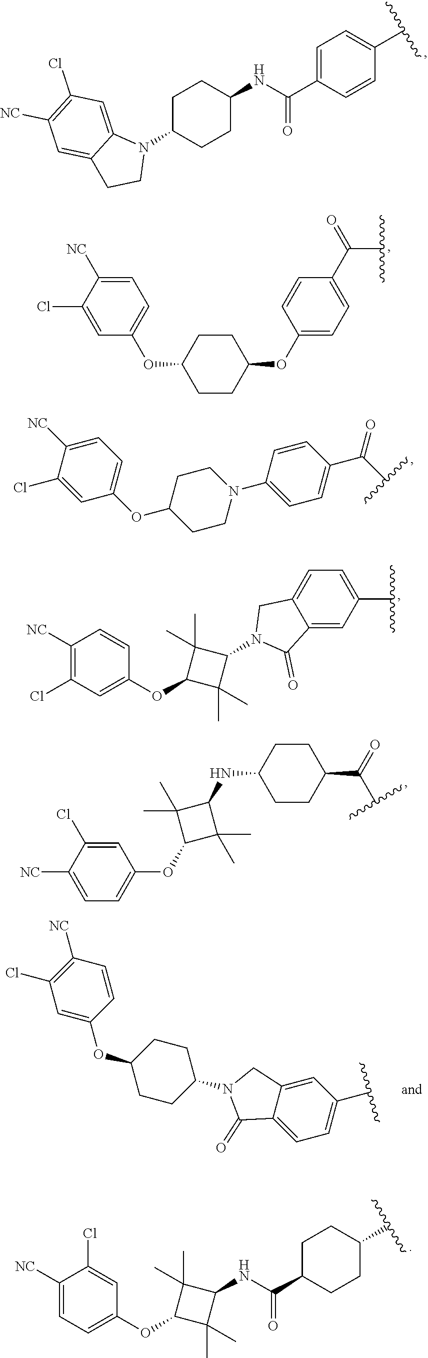

### R¹

In some embodiments of the compound of formula (I), (I-A), (I-B), (I-C), (I-D), (I-A-1), (I-A-2), (I-A-3), (I-A-4), (I-B-1), (I-B-2), (I-B-3), (I-B-4), (I-C-1), (I-C-2), (I-C-3), (I-C-4), (I-D-1), (I-D-2), (I-D-3), (I-D-4), (I-A-1-a), (I-A-2-a), (I-A-3-a), (I-B-1-a), (I-B-2-a), (I-B-3-a), (I-C-1-a), (I-D-1-a), (II), (II-A), (II-B), (II-C), (II-D), (II-A-1), (II-A-2), (II-A-3), (II-A-4), (II-B3-1), (II-B3-2), (II-B3-3), (II-B3-4), (II-C-1), (II-C-2), (II-C-3), (II-C-4), (II-D-1), (II-D-2), (II-D-3), (II-D-4), (III), (III-A), (III-B), (III-C), (III-D), (III-A-1), (III-A-2), (III-A-3), (III-A-4), (III-B-1), (III-B-2), (III-B-3), (III-B-4), (III-C-1), (III-C-2), (III-C-3), (III-C-4), (III-D-1), (III-D-2), (III-D-3), or (III-D-4), or a pharmaceutically acceptable salt, stereoisomer, or deuterated form thereof, each R¹ is independently optionally substituted C₁₋₆ alkyl, optionally substituted C₃-C₈ cycloalkyl, or halogen. In some embodiments, each R¹ is independently optionally substituted C₁₋₆ alkyl, C₂₋₅ alkyl, or C₃₋₄ alkyl. In some embodiments, each R¹ is independently optionally substituted C₃-C₈ cycloalkyl, C₄-C₇ cycloalkyl, or C₅-C₆ cycloalkyl. In some embodiments, each R¹ is independent halogen.

In some embodiments of the compound of formula (I), (I-A), (I-B), (I-C), (I-D), (I-A-1), (I-A-2), (I-A-3), (I-A-4), (I-B-1), (I-B-2), (I-B-3), (I-B-4), (I-C-1), (I-C-2), (I-C-3), (I-C-4), (I-D-1), (I-D-2), (I-D-3), (I-D-4), (I-A-1-a), (I-A-2-a), (I-A-3-a), (I-B-1-a), (I-B-2-a), (I-B-3-a), (I-C-1-a), (I-D-1-a), (II), (II-A), (II-B), (II-C), (II-D), (II-A-1), (II-A-2), (II-A-3), (II-A-4), (II-B-1), (II-B-2), (II-B-3), (II-B-4), (II-C-1), (II-C-2), (II-C-3), (II-C-4), (II-D-1), (II-D-2), (II-D-3), (II-D-4), (III), (III-A), (III-C), (III-D), (III-A-1), (III-A-2), (III-A-3), (III-A-4), (III-B-1), (III-B-2), (III-B-3), (III-B-4), (III-C-1), (III-C-2), (III-C-3), (III-C-4), (III-D-1), (III-D-2), (III-D-3), or (III-D-4), or a pharmaceutically acceptable salt, stereoisomer, or deuterated form thereof, each R¹ is —F, —C₁, —Br, or —I. In some embodiments, each R¹ is —F or —Cl. In some embodiments, each R¹ is —F. In some embodiments, each R¹ is —Cl.

In some embodiments of the compound of formula (I), (I-A), (I-B), (I-C), (I-D), (I-A-1), (I-A-2), (I-A-3), (I-A-4), (I-B-1), (I-B-2), (I-B-3), (I-B-4), (I-C-1), (I-C-2), (I-C-3), (I-C-4), (I-D-1), (I-D-2), (I-D-3), (I-D-4), (I-A-1-a), (I-A-2-a), (I-A-3-a), (I-B-1-a), (I-B-2-a), (I-B-3-a), (I-C-1-a), (I-D-1-a), (II), (II-A), (II-B), (II-C), (II-D), (II-A-1), (II-A-2), (II-A-3), (II-A-4), (II-B3-1), (II-B3-2), (II-B3-3), (II-B3-4), (II-C-1), (II-C-2), (II-C-3), (II-C-4), (II-D-1), (II-D-2), (II-D-3), (II-D-4), (III), (III-A), (III-B), (III-C), (III-D), (III-A-1), (III-A-2), (III-A-3), (III-A-4), (III-B-1), (III-B-2), (III-B-3), (III-B-4), (III-C-1), (III-C-2), (III-C-3), (III-C-4), (III-D-1), (III-D-2), (III-D-3), or (III-D-4), or a pharmaceutically acceptable salt, stereoisomer, or deuterated form thereof, each R¹ is C₁₋₆ alkyl. In some embodiments, each R¹ is C₁₋₃ alkyl. In some embodiments, each R¹ is methyl.

a

In some embodiments of the compound of formula (I), (I-A), (I-B), (I-C), (I-D), (I-A-1), (I-A-2), (I-A-3), (I-A-4), (I-B-1), (I-B-2), (I-B-3), (I-B-4), (I-C-1), (I-C-2), (I-C-3), (I-C-4), (I-D-1), (I-D-2), (I-D-3), (I-D-4), (I-A-1-a), (I-A-2-a), (I-A-3-a), (I-B-1-a), (I-B-2-a), (I-B-3-a), (I-C-1-a), (I-D-1-a), (II), (II-A), (II-B), (II-C), (II-D), (II-A-1), (II-A-2), (II-A-3), (II-A-4), (II-B-1), (II-B-2), (II-B1-3), (II-B-4), (II-C-1), (II-C-2), (II-C-3), (II-C-4), (II-D-1), (II-D-2), (II-D-3), (II-D-4), (III), (III-A), (III-B), (III-C), (III-D), (III-A-1), (III-A-2), (III-A-3), (III-A-4), (III-B-1), (III-B-2), (III-B-3), (III-B-4), (III-C-1), (III-C-2), (III-C-3), (III-C-4), (III-D-1), (III-D-2), (III-D-3), or (III-D-4), or a pharmaceutically acceptable salt, stereoisomer, or deuterated form thereof, a is an integer of 0-3. In some embodiments, a is 0, 1, 2, or 3. In some embodiments, a is 1 or 2. In some embodiments, a is 0 or 1. In some embodiments, a is 0. In some embodiments, a is 1.

### R²

In some embodiments of the compound of formula (I), (I-A), (I-B), (I-A-1), (I-A-2), (I-A-3), (I-A-4), (I-B-1), (I-B-2), (I-B-3), (I-B-4), (I-A-1-a), (I-A-2-a), (I-A-3-a), (I-B-1-a), (I-B-2-a), (I-B-3-a), (I-C-1-a), (I-D-1-a), (II), (II-A), (II-B), (II-A-1), (II-A-2), (II-A-3), (II-A-4), (II-B3-1), (II-B3-2), (II-B3-3), (II-B3-4), (III), (III-A), (III-B), (III-A-1), (III-A-2), (III-A-3), (III-A-4), (III-B3-1), (III-B-2), (III-B-3), or (III-B-4), or a pharmaceutically acceptable salt, stereoisomer, or deuterated form thereof, each R² is independently optionally substituted C₁₋₆ alkyl, optionally substituted C₃-C₈ cycloalkyl, or halogen. In some embodiments, each R² is independently optionally substituted C₁₋₆ alkyl, C₂₋₅ alkyl, or C₃₋₄ alkyl. In some embodiments, each R² is independently optionally substituted C₃-C₈ cycloalkyl, C₄-C₇ cycloalkyl, or C₅-C₆ cycloalkyl. In some embodiments, each R² is independently halogen. In some embodiments, each R² is independently C₁₋₆ alkyl, C₂₋₅ alkyl, or C₃₋₄ alkyl. In embodiments, each R² is independently optionally substituted C₃-C₈ cycloalkyl or halogen. In embodiments, R² is not optionally substituted C₁₋₆ alkyl. In embodiments, R² is not methyl. In embodiments, R² is absent.

In some embodiments of the compound of formula (I), (I-A), (I-B), (I-A-1), (I-A-2), (I-A-3), (I-A-4), (I-B-1), (I-B-2), (I-B-3), (I-B-4), (I-A-1-a), (I-A-2-a), (I-A-3-a), (I-B-1-a), (I-B-2-a), (I-B-3-a), (I-C-1-a), (I-D-1-a), (II), (II-A), (II-B), (II-A-1), (II-A-2), (II-A-3), (II-A-4), (II-B3-1), (II-B3-2), (II-B3-3), (II-B3-4), (III), (III-A), (III-B), (III-A-1), (III-A-2), (III-A-3), (III-A-4), (III-B-1), (III-B-2), (III-B-3), or (III-B-4), or a pharmaceutically acceptable salt, stereoisomer, or deuterated form thereof, each R² is independently C₁₋₃ alkyl (e.g., methyl, ethyl, n-propyl, isopropyl).

In some embodiments of the compound of formula (I), (I-A), (I-B), (I-A-1), (I-A-2), (I-A-3), (I-A-4), (I-B-1), (I-B-2), (I-B-3), (I-B-4), (I-A-1-a), (I-A-2-a), (I-A-3-a), (I-B-1-a), (I-B-2-a), (I-B-3-a), (I-C-1-a), (I-D-1-a), (II), (II-A), (II-B), (II-A-1), (II-A-2), (II-A-3), (II-A-4), (II-B3-1), (II-B3-2), (II-B3-3), (II-B3-4), (III), (III-A), (III-B), (III-A-1), (III-A-2), (III-A-3), (III-A-4), (III-B-1), (III-B-2), (III-B-3), or (III-B-4), or a pharmaceutically acceptable salt, stereoisomer, or deuterated form thereof, each R² is methyl.

b

In some embodiments of the compound of formula (I), (I-A), (I-B), (I-C), (I-D), (I-A-1), (I-A-2), (I-A-3), (I-A-4), (I-B-1), (I-B-2), (I-B-3), (I-B-4), (I-C-1), (I-C-2), (I-C-3), (I-C-4), (I-D-1), (I-D-2), (I-D-3), (I-D-4), (I-A-1-a), (I-A-2-a), (I-A-3-a), (I-B-1-a), (I-B-2-a), (I-B-3-a), (I-C-1-a), (I-D-1-a), (II), (II-A), (II-B), (II-C), (II-D), (II-A-1), (II-A-2), (II-A-3), (II-A-4), (II-B3-1), (II-B3-2), (II-B3-3), (II-B3-4), (II-C-1), (II-C-2), (II-C-3), (II-C-4), (II-D-1), (II-D-2), (II-D-3), (II-D-4), (III), (III-A), (III-B), (III-C), (III-D), (III-A-1), (III-A-2), (III-A-3), (III-A-4), (III-B-1), (III-B-2), (III-B-3), (III-B-4), (III-C-1), (III-C-2), (III-C-3), (III-C-4), (III-D-1), (III-D-2), (III-D-3), or (III-D-4), or a pharmaceutically acceptable salt, stereoisomer, or deuterated form thereof, b is an integer of 0-2. In some embodiments, b is 0, 1, or 2. In some embodiments,
b is 1 or 2. In some embodiments, b is 0 or 1. In some embodiments, b is 0. In some embodiments, b is 1.

Q

In some embodiments of the compound of formula (I), (I-A), (I-B), (I-C), (I-D), (I-A-1), (I-A-2), (I-A-3), (I-A-4), (I-B-1), (I-B-2), (I-B-3), (I-B-4), (I-C-1), (I-C-2), (I-C-3), (I-C-4), (I-D-1), (I-D-2), (I-D-3), (I-D-4), (I-A-1-a), (I-A-2-a), (I-A-3-a), (I-B-1-a), (I-B-2-a), (I-B-3-a), (I-C-1-a), (I-D-1-a), (II), (II-A), (II-B), (II-C), (II-D), (II-A-1), (II-A-2), (II-A-3), (II-A-4), (II-B3-1), (II-B3-2), (II-B3-3), (II-B3-4), (II-C-1), (II-C-2), (II-C-3), (II-C-4), (II-D-1), (II-D-2), (II-D-3), (II-D-4), (III), (III-A), (III-B), (III-C), (III-D), (III-A-1), (III-A-2), (III-A-3), (III-A-4), (III-B-1), (III-B-2), (III-B-3), (III-B-4), (III-C-1), (III-C-2), (III-C-3), (III-C-4), (III-D-1), (III-D-2), (III-D-3), or (III-D-4), or a pharmaceutically acceptable salt, stereoisomer, or deuterated form thereof, Q is absent, C₁₋₆ alkylene, —C₁₋₆ alkylene-C(O)—, C₃₋₈ cycloalkylene, —C₃₋₈ cycloalkylene-C(O)—, —C(O)—, or —S(O)₂—, wherein the alkylene is optionally and independently substituted. In some embodiments, Q is a absent, C₁₋₄ alkylene, —(C₁₋₃ alkylene)C(O)—, C₃₋₆ cycloalkylene, —(C₃₋₆ cycloalkylene)C(O)—, —C(O)—, or —S(O)₂—, wherein the alkylene is optionally substituted. In some embodiments, Q is a absent. In embodiments, Q is absent, C₁₋₆ alkylene, —C₁₋₆ alkylene-C(O)—, C₃₋₈ cycloalkylene, —C₃₋₈ cycloalkylene-C(O)—, or —S(O)₂—, wherein the alkylene is optionally and independently substituted. In embodiments, Q is not —C(O)—.

In some embodiments, Q is C₁₋₁₂ alkylene, C₁₋₆ alkylene, C₁₋₅ alkylene, C₁₋₄ alkylene, C₁₋₃ alkylene, or C₁₋₂ alkylene. In some embodiments, Q is —(C₁₋₆ alkylene)C(O)—, —(C₁₋₃ alkylene)C(O)—, —(C₁₋₂ alkylene)C(O)—, or —CH₂—C(O)—. In some embodiments, Q is C₃₋₉ cycloalkylene, C₃₋₆ cycloalkylene, C₃₋₅ cycloalkylene, or C₃₋₄ cycloalkylene. In some embodiments, Q is —(C₃₋₉ cycloalkylene)C(O)—, —(C₃₋₆ cycloalkylene)C(O)—, —(C₃₋₅ cycloalkylene)C(O)—, or —(C₃₋₄ cycloalkylene)C(O)—. In some embodiments, Q is —C(O)—. In some embodiments, Q is —S(O)₂—.

In some embodiments of the compound of formula (I), (I-A), (I-B), (I-C), (I-D), (I-A-1), (I-A-2), (I-A-3), (I-A-4), (I-B-1), (I-B-2), (I-B-3), (I-B-4), (I-C-1), (I-C-2), (I-C-3), (I-C-4), (I-D-1), (I-D-2), (I-D-3), (I-D-4), (I-A-1-a), (I-A-2-a), (I-A-3-a), (I-B-1-a), (I-B-2-a), (I-B-3-a), (I-C-1-a), (I-D-1-a), (II), (II-A), (II-B), (II-C), (II-D), (II-A-1), (II-A-2), (II-A-3), (II-A-4), (II-B3-1), (II-B3-2), (II-B3-3), (II-B3-4), (II-C-1), (II-C-2), (II-C-3), (II-C-4), (II-D-1), (II-D-2), (II-D-3), (II-D-4), (III), (III-A), (III-B), (III-C), (III-D), (III-A-1), (III-A-2), (III-A-3), (III-A-4), (III-B-1), (III-B-2), (III-B-3), (III-B-4), (III-C-1), (III-C-2), (III-C-3), (III-C-4), (III-D-1), (III-D-2), (III-D-3), or (III-D-4), or a pharmaceutically acceptable salt, stereoisomer, or deuterated form thereof, Q is a absent.

In some embodiments of the compound of formula (I), (I-A), (I-B), (I-C), (I-D), (I-A-1), (I-A-2), (I-A-3), (I-A-4), (I-B-1), (I-B-2), (I-B-3), (I-B-4), (I-C-1), (I-C-2), (I-C-3), (I-C-4), (I-D-1), (I-D-2), (I-D-3), (I-D-4), (I-A-1-a), (I-A-2-a), (I-A-3-a), (I-B-1-a), (I-B-2-a), (I-B-3-a), (I-C-1-a), (I-D-1-a), (II), (II-A), (II-B), (II-C), (II-D), (II-A-1), (II-A-2), (II-A-3), (II-A-4), (II-B3-1), (II-B3-2), (II-B3-3), (II-B3-4), (II-C-1), (II-C-2), (II-C-3), (II-C-4), (II-D-1), (II-D-2), (II-D-3), (II-D-4), (III), (III-A), (III-B), (III-C), (III-D), (III-A-1), (III-A-2), (III-A-3), (III-A-4), (III-B-1), (III-B-2), (III-B-3), (III-B-4), (III-C-1), (III-C-2), (III-C-3), (III-C-4), (III-D-1), (III-D-2), (III-D-3), or (III-D-4), or a pharmaceutically acceptable salt, stereoisomer, or deuterated form thereof, Q is —CH₂—, —CH₂CH₂—, —CH(CH₃)—, or —C(CH₃)₂—. In some embodiments, Q is —CH₂—. In some embodiments, Q is —CH₂CH₂—.

In some embodiments of the compound of formula (I), (I-A), (I-B), (I-C), (I-D), (I-A-1), (I-A-2), (I-A-3), (I-A-4), (I-B-1), (I-B-2), (I-B-3), (I-B-4), (I-C-1), (I-C-2), (I-C-3), (I-C-4), (I-D-1), (I-D-2), (I-D-3), (I-D-4), (I-A-1-a), (I-A-2-a), (I-A-3-a), (I-B-1-a), (I-B-2-a), (I-B-3-a), (I-C-1-a), (I-D-1-a), (II), (II-A), (II-B), (II-C), (II-D), (II-A-1), (II-A-2), (II-A-3), (II-A-4), (II-B3-1), (II-B3-2), (II-B3-3), (II-B3-4), (II-C-1), (II-C-2), (II-C-3), (II-C-4), (II-D-1), (II-D-2), (II-D-3), (II-D-4), (III), (III-A), (III-B), (III-C), (III-D), (III-A-1), (III-A-2), (III-A-3), (III-A-4), (III-B-1), (III-B-2), (III-B-3), (III-B-4), (III-C-1), (III-C-2), (III-C-3), (III-C-4), (III-D-1), (III-D-2), (III-D-3), or (III-D-4), or a pharmaceutically acceptable salt, stereoisomer, or deuterated form thereof, Q is —C(O)—.

In some embodiments of the compound of formula (I), (I-A), (I-B), (I-C), (I-D), (I-A-1), (I-A-2), (I-A-3), (I-A-4), (I-B-1), (I-B-2), (I-B-3), (I-B-4), (I-C-1), (I-C-2), (I-C-3), (I-C-4), (I-D-1), (I-D-2), (I-D-3), (I-D-4), (I-A-1-a), (I-A-2-a), (I-A-3-a), (I-B-1-a), (I-B-2-a), (I-B-3-a), (I-C-1-a), (I-D-1-a), (II), (II-A), (II-B), (II-C), (II-D), (II-A-1), (II-A-2), (II-A-3), (II-A-4), (II-B3-1), (II-B3-2), (II-B3-3), (II-B3-4), (II-C-1), (II-C-2), (II-C-3), (II-C-4), (II-D-1), (II-D-2), (II-D-3), (II-D-4), (III), (III-A), (III-B), (III-C), (III-D), (III-A-1), (III-A-2), (III-A-3), (I-A-4), (III-B-1), (III-B-2), (III-B-3), (III-B-4), (III-C-1), (III-C-2), (III-C-3), (III-C-4), (III-D-1), (III-D-2), (III-D-3), or (III-D-4), or a pharmaceutically acceptable salt, stereoisomer, or deuterated form thereof, Q is —S(O)₂—.

In some embodiments of the compound of formula (I), (I-A), (I-B), (I-C), (I-D), (I-A-1), (I-A-2), (I-A-3), (I-A-4), (I-B-1), (I-B-2), (I-B-3), (I-B-4), (I-C-1), (I-C-2), (I-C-3), (I-C-4), (I-D-1), (I-D-2), (I-D-3), (I-D-4), (I-A-1-a), (I-A-2-a), (I-A-3-a), (I-B-1-a), (I-B-2-a), (I-B-3-a), (I-C-1-a), (I-D-1-a), (II), (II-A), (II-B), (II-C), (II-D), (II-A-1), (II-A-2), (II-A-3), (II-A-4), (II-B3-1), (II-B3-2), (II-B3-3), (II-B3-4), (II-C-1), (II-C-2), (II-C-3), (II-C-4), (II-D-1), (II-D-2), (II-D-3), (II-D-4), (III), (III-A), (III-B), (III-C), (III-D), (III-A-1), (III-A-2), (III-A-3), (III-A-4), (III-B-1), (III-B-2), (III-B-3), (III-B-4), (III-C-1), (III-C-2), (III-C-3), (III-C-4), (III-D-1), (III-D-2), (III-D-3), or (III-D-4), or a pharmaceutically acceptable salt, stereoisomer, or deuterated form thereof, Q is

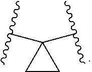 [SMILES: *C1(*)CC1]

In some embodiments of the compound of formula (I), (I-A), (I-B), (I-C), (I-D), (I-A-1), (I-A-2), (I-A-3), (I-A-4), (I-B-1), (I-B-2), (I-B-3), (I-B-4), (I-C-1), (I-C-2), (I-C-3), (I-C-4), (I-D-1), (I-D-2), (I-D-3), (I-D-4), (I-A-1-a), (I-A-2-a), (I-A-3-a), (I-B-1-a), (I-B-2-a), (I-B-3-a), (I-C-1-a), (I-D-1-a), (II), (II-A), (II-B), (II-C), (II-D), (II-A-1), (II-A-2), (II-A-3), (II-A-4), (II-B3-1), (II-B3-2), (II-B3-3), (II-B3-4), (II-C-1), (II-C-2), (II-C-3), (II-C-4), (II-D-1), (II-D-2), (II-D-3), (II-D-4), (III), (III-A), (III-B), (III-C), (III-D), (III-A-1), (III-A-2), (III-A-3), (III-A-4), (III-B-1), (III-B-2), (III-B-3), (III-B-4), (III-C-1), (III-C-2), (III-C-3), (III-C-4), (III-D-1), (III-D-2), (III-D-3), or (III-D-4), or a pharmaceutically acceptable salt, stereoisomer, or deuterated form thereof, Q is —CH₂C(O)—.

In some embodiments of the compound of formula (I), (I-A), (I-B), (I-C), (I-D), (I-A-1), (I-A-2), (I-A-3), (I-A-4), (I-B-1), (I-B-2), (I-B-3), (I-B-4), (I-C-1), (I-C-2), (I-C-3), (I-C-4), (I-D-1), (I-D-2), (I-D-3), (I-D-4), (I-A-1-a), (I-A-2-a), (I-A-3-a), (I-B-1-a), (I-B-2-a), (I-B-3-a), (I-C-1-a), (I-D-1-a), (II), (II-A), (II-B), (II-C), (II-D), (II-A-1), (II-A-2), (II-A-3), (II-A-4), (II-B3-1), (II-B3-2), (II-B3-3), (II-B3-4), (II-C-1), (II-C-2), (II-C-3), (II-C-4), (II-D-1), (II-D-2), (II-D-3), (II-D-4), (III), (III-A), (III-B), (III-C), (III-D), (III-A-1), (III-A-2), (III-A-3), (III-A-4), (III-B3-1), (III-B-2), (III-B-3), (III-B-4), (III-C-1), (III-C-2), (III-C-3), (III-C-4), (III-D-1), (III-D-2), (III-D-3), or (III-D-4), or a pharmaceutically acceptable salt, stereoisomer, or deuterated form thereof, Q is

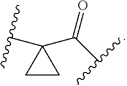 [SMILES: *C(=O)C1(*)CC1]

R³ and R⁴

In some embodiments of the compound of formula (I), (I-A), (I-B), (I-C), (I-D), (I-A-1), (I-A-2), (I-A-3), (I-A-4), (I-B-1), (I-B-2), (I-B-3), (I-B-4), (I-C-1), (I-C-2), (I-C-3), (I-C-4), (I-D-1), (I-D-2), (I-D-3), (I-D-4), (I-A-1-a), (I-A-2-a), (I-A-3-a), (I-B-1-a), (I-B-2-a), (I-B-3-a), (I-C-1-a), (I-D-1-a), (II), (II-A), (II-B), (II-C), (II-D), (II-A-1), (II-A-2), (II-A-3), (II-A-4), (II-B3-1), (II-B3-2), (II-B3-3), (II-B3-4), (II-C-1), (II-C-2), (II-C-3), (II-C-4), (II-D-1), (II-D-2), (II-D-3), (II-D-4), (III), (III-A), (III-B), (III-C), (III-D), (III-A-1), (III-A-2), (III-A-3), (III-A-4), (III-B-1), (III-B-2), (III-B-3), (III-B-4), (III-C-1), (III-C-2), (III-C-3), (III-C-4), (III-D-1), (III-D-2), (III-D-3), or (III-D-4), or a pharmaceutically acceptable salt, stereoisomer, or deuterated form thereof, R³ is carbocycle, heterocycle, aryl, heteroaryl, or —NR^{Y}, wherein the carbocycle, heterocycle, aryl, and heteroaryl are each optionally substituted, R^{X} is H, C₁₋₆ alkyl, C₁₋₆ haloalkyl, or C₁₋₆ hydroxyalkyl, and R^{Y} is C₁₋₆ alkyl, C₃₋₈ cycloalkyl, C₃₋₈ hydroxycycloalkyl, aryl, or heterocycle. In some embodiments, the carbocycle, heterocycle, aryl, and heteroaryl are each optionally substituted with 1, 2, or 3 R⁴, wherein each R⁴ is independently halogen, —OH, C₁₋₆ alkyl, C₁₋₆ alkoxy, —C(O)—NR^{X}R^{Y}, or —C(O)O—(C₁₋₆ alkyl). In embodiments, R³ is not a tetrazolyl.

In some embodiments of the compound of formula (I), (I-A), (I-B), (I-C), (I-D), (I-A-1), (I-A-2), (I-A-3), (I-A-4), (I-B-1), (I-B-2), (I-B-3), (I-B-4), (I-C-1), (I-C-2), (I-C-3), (I-C-4), (I-D-1), (I-D-2), (I-D-3), (I-D-4), (I-A-1-a), (I-A-2-a), (I-A-3-a), (I-B-1-a), (I-B-2-a), (I-B-3-a), (I-C-1-a), (I-D-1-a), (II), (II-A), (II-B), (II-C), (II-D), (II-A-1), (II-A-2), (II-A-3), (II-A-4), (II-B3-1), (II-B3-2), (II-B3-3), (II-B3-4), (II-C-1), (II-C-2), (II-C-3), (II-C-4), (II-D-1), (II-D-2), (II-D-3), (II-D-4), (III), (III-A), (III-B), (III-C), (III-D), (III-A-1), (III-A-2), (III-A-3), (III-A-4), (III-B-1), (III-B-2), (III-B-3), (III-B-4), (III-C-1), (III-C-2), (III-C-3), (III-C-4), (III-D-1), (III-D-2), (III-D-3), or (III-D-4), or a pharmaceutically acceptable salt, stereoisomer, or deuterated form thereof, R³ is C₃₋₈ carbocycle, 3-10 membered heterocycle, C₆₋₁₀ aryl, 5-10 membered heteroaryl, or —NR^{X}R^{Y}, wherein the C₃₋₈ carbocycle, 3-8 membered heterocycle, C₆₋₁₀ aryl, and 5-10 membered heteroaryl are each optionally substituted, and wherein R^{X} is H, C₁₋₆ alkyl, C₁₋₆ haloalkyl, or C₁₋₆ hydroxyalkyl, and R^{Y} is C₁₋₆ alkyl, C₃₋₈ cycloalkyl, C₃₋₈ hydroxycycloalkyl, aryl, or heterocycle. In some embodiments, the C₃₋₈ carbocycle, 3-10 membered heterocycle, C₆₋₁₀ aryl, and 5-10 membered heteroaryl are each optionally substituted with 1 or 2 R⁴, and each R⁴ is independently halogen, —OH, C₁₋₆ alkyl, C₁₋₆ alkoxy, —C(O)—NR^{X}R^{Y}, or —C(O)O—C₁₋₆ alkyl.

In some embodiments of the compound of formula (I), (I-A), (I-B), (I-C), (I-D), (I-A-1), (I-A-2), (I-A-3), (I-A-4), (I-B-1), (I-B-2), (I-B-3), (I-B-4), (I-C-1), (I-C-2), (I-C-3), (I-C-4), (I-D-1), (I-D-2), (I-D-3), (I-D-4), (I-A-1-a), (I-A-2-a), (I-A-3-a), (I-B-1-a), (I-B-2-a), (I-B-3-a), (I-C-1-a), (I-D-1-a), (II), (II-A), (II-B), (II-C), (II-D), (II-A-1), (II-A-2), (II-A-3), (II-A-4), (II-B-1), (II-B-2), (II-B-3), (II-B-4), (II-C-1), (II-C-2), (II-C-3), (II-C-4), (II-D-1), (II-D-2), (II-D-3), (II-D-4), (III), (III-A), (III-B), (III-C), (III-D), (II-A-1), (II-A-2), (II-A-3), (III-A-4), (III-B-1), (III-B-2), (III-B-3), (III-B-4), (III-C-1), (III-C-2), (III-C-3), (III-C-4), (III-D-1), (III-D-2), (III-D-3), or (III-D-4), or a pharmaceutically acceptable salt, stereoisomer, or deuterated form thereof, R³ is C₆₋₁₀ aryl optionally substituted with 1 or 2 R⁴, and wherein each R⁴ is independently halogen, C₁₋₆ alkyl, C₁₋₆ alkoxy, or —C(O)—NR^{X}R^{Y}.

In some embodiments of the compound of formula (I), (I-A), (I-B), (I-C), (I-D), (I-A-1), (I-A-2), (I-A-3), (I-A-4), (I-B-1), (I-B-2), (I-B-3), (I-B-4), (I-C-1), (I-C-2), (I-C-3), (I-C-4), (I-D-1), (I-D-2), (I-D-3), (I-D-4), (I-A-1-a), (I-A-2-a), (I-A-3-a), (I-B-1-a), (I-B-2-a), (I-B-3-a), (I-C-1-a), (I-D-1-a), (II), (II-A), (II-B), (II-C), (II-D), (II-A-1), (II-A-2), (II-A-3), (II-A-4), (II-B3-1), (II-B3-2), (II-B3-3), (II-B3-4), (II-C-1), (II-C-2), (II-C-3), (II-C-4), (II-D-1), (II-D-2), (II-D-3), (II-D-4), (III), (III-A), (III-B), (III-C), (III-D), (III-A-1), (III-A-2), (III-A-3), (III-A-4), (III-B-1), (III-B-2), (III-B-3), (III-B-4), (III-C-1), (III-C-2), (III-C-3), (III-C-4), (III-D-1), (III-D-2), (III-D-3), or (III-D-4), or a pharmaceutically acceptable salt, stereoisomer, or deuterated form thereof, R³ is C₆₋₁₀ aryl optionally substituted with —F, —C₁, C₁₋₃ alkoxy, or —C(O)—NR^{X}R^{Y}, wherein R^{X} is H or C₁₋₆ alkyl, and R^{Y} is C₁₋₆ alkyl. In some embodiments, R³ is

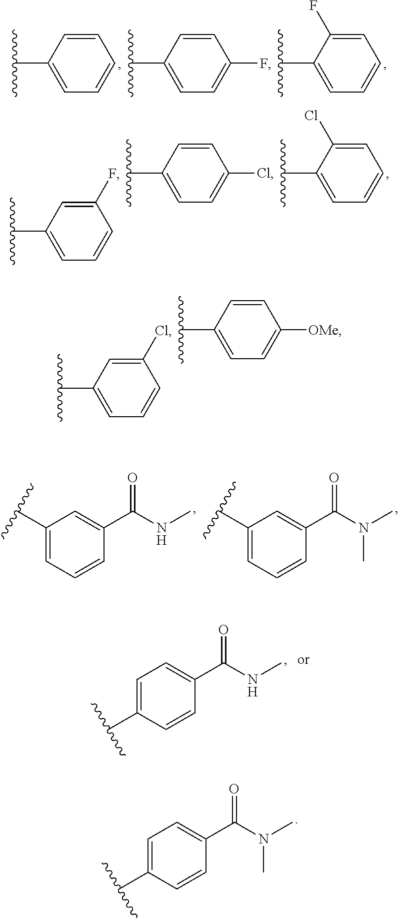

In embodiments, R³ is

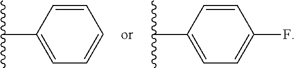 [SMILES: *c1ccccc1 ; *c1ccc(F)cc1]

In some embodiments of the compound of formula (I), (I-A), (I-B), (I-C), (I-D), (I-A-1), (I-A-2), (I-A-3), (I-A-4), (I-B-1), (I-B-2), (I-B-3), (I-B-4), (I-C-1), (I-C-2), (I-C-3), (I-C-4), (I-D-1), (I-D-2), (I-D-3), (I-D-4), (I-A-1-a), (I-A-2-a), (I-A-3-a), (I-B-1-a), (I-B-2-a), (I-B-3-a), (I-C-1-a), (I-D-1-a), (II), (II-A), (II-B), (II-C), (II-D), (II-A-1), (II-A-2), (II-A-3), (II-A-4), (II-B3-1), (II-B3-2), (II-B3-3), (II-B3-4), (II-C-1), (II-C-2), (II-C-3), (II-C-4), (II-D-1), (II-D-2), (II-D-3), (II-D-4), (III), (III-A), (III-B), (III-C), (III-D), (III-A-1), (III-A-2), (III-A-3), (III-A-4), (III-B-1), (III-B-2), (III-B-3), (III-B-4), (III-C-1), (III-C-2), (III-C-3), (III-C-4), (III-D-1), (III-D-2), (III-D-3), or (III-D-4), or a pharmaceutically acceptable salt, stereoisomer, or deuterated form thereof, R³ is 5-10 membered heteroaryl optionally substituted with 1 or 2 R⁴; and wherein the heteroaryl contains 1 or 2 heteroatoms selected from N, S, or O. In some embodiments, R³

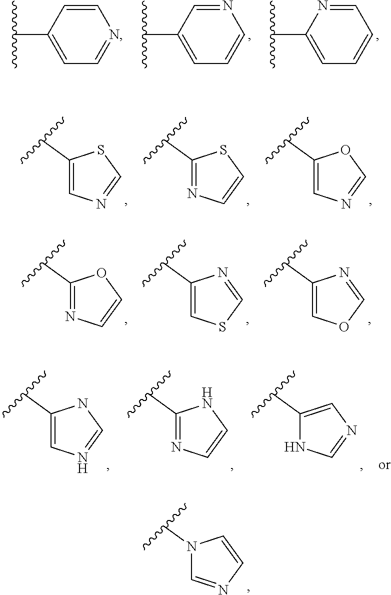

each optionally substituted with one R⁴. In some embodiments R³ is

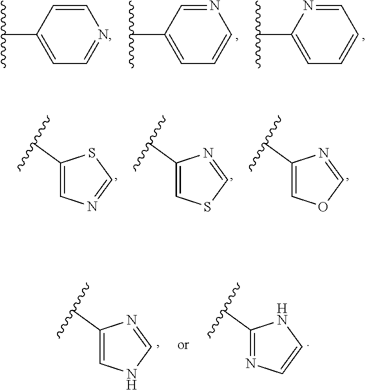

In some embodiments R³ is

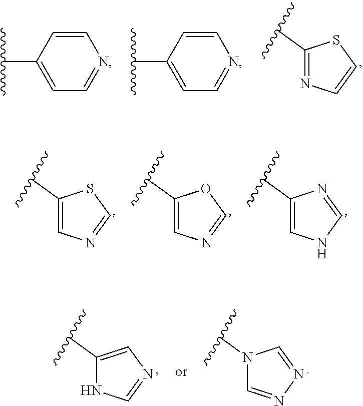

In some embodiments of the compound of formula (I), (I-A), (I-B), (I-C), (I-D), (I-A-1), (I-A-2), (I-A-3), (I-A-4), (I-B-1), (I-B-2), (I-B-3), (I-B-4), (I-C-1), (I-C-2), (I-C-3), (I-C-4), (I-D-1), (I-D-2), (I-D-3), (I-D-4), (I-A-1-a), (I-A-2-a), (I-A-3-a), (I-B-1-a), (I-B-2-a), (I-B-3-a), (I-C-1-a), (I-D-1-a), (II), (II-A), (II-B), (II-C), (II-D), (II-A-1), (II-A-2), (II-A-3), (II-A-4), (II-B3-1), (II-B3-2), (II-B3-3), (II-B3-4), (II-C-1), (II-C-2), (II-C-3), (II-C-4), (II-D-1), (II-D-2), (II-D-3), (II-D-4), (III), (III-A), (III-B), (III-C), (III-D), (III-A-1), (III-A-2), (III-A-3), (III-A-4), (III-B3-1), (III-B-2), (III-B-3), (III-B-4), (III-C-1), (III-C-2), (III-C-3), (III-C-4), (III-D-1), (III-D-2), (III-D-3), or (III-D-4), or a pharmaceutically acceptable salt, stereoisomer, or deuterated form thereof, R³ is C₃₋₈ carbocycle optionally substituted with 1 or 2 R⁴, and wherein each R⁴ is independently halogen, —OH, C₁₋₆ alkyl, or C₁₋₆ alkoxy. In some embodiments, R³ is

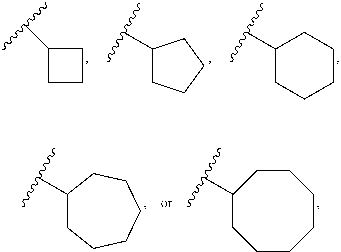

each optionally substituted with one or two R⁴. In some embodiments, R⁴ is halogen. In some embodiments, each R⁴ is fluoro. In some embodiments, R³ is

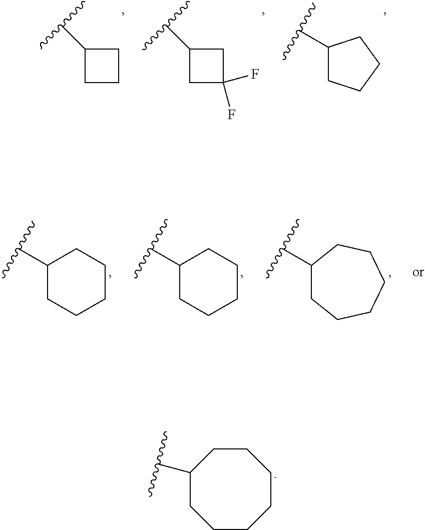

In some embodiments, R³ is

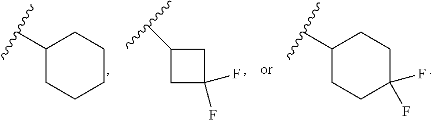 [SMILES: *C1CCCCC1 ; *C1CC(F)(F)C1 ; *C1CCC(F)(F)CC1]

In some embodiments, R³ is

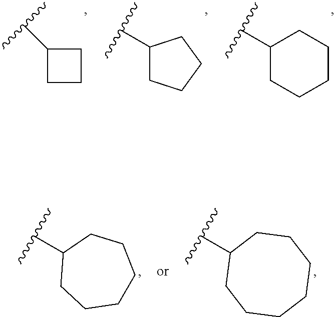

each optionally substituted with one R⁴. In some embodiments, R³ is

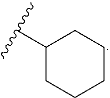 [SMILES: *C1CCCCC1]

In some embodiments of the compound of formula (I), (I-A), (I-B), (I-C), (I-D), (I-A-1), (I-A-2), (I-A-3), (I-A-4), (I-B-1), (I-B-2), (I-B-3), (I-B-4), (I-C-1), (I-C-2), (I-C-3), (I-C-4), (I-D-1), (I-D-2), (I-D-3), (I-D-4), (I-A-1-a), (I-A-2-a), (I-A-3-a), (I-B-1-a), (I-B-2-a), (I-B-3-a), (I-C-1-a), (I-D-1-a), (II), (II-A), (II-B), (II-C), (II-D), (II-A-1), (II-A-2), (II-A-3), (II-A-4), (II-B3-1), (II-B3-2), (II-B3-3), (II-B3-4), (II-C-1), (II-C-2), (II-C-3), (II-C-4), (II-D-1), (II-D-2), (II-D-3), (II-D-4), (III), (III-A), (III-B), (III-C), (III-D), (III-A-1), (III-A-2), (III-A-3), (III-A-4), (III-B-1), (III-B-2), (III-B-3), (III-B-4), (III-C-1), (III-C-2), (III-C-3), (III-C-4), (III-D-1), (III-D-2), (III-D-3), or (III-D-4), or a pharmaceutically acceptable salt, stereoisomer, or deuterated form thereof, R³ is 3-10 membered heterocycle containing 1 or 2 heteroatoms selected from N, O, or S, and the heterocycle is optionally substituted with 1 or 2 R⁴, and wherein each R⁴ is independently —OH, C₁₋₆ alkyl, or —C(O)O—(C₁₋₆ alkyl). In some embodiments, each R⁴ is independently —OH, C₁₋₃ alkyl, or —C(O)O—(C₁₋₅ alkyl). In some embodiments, R³ is

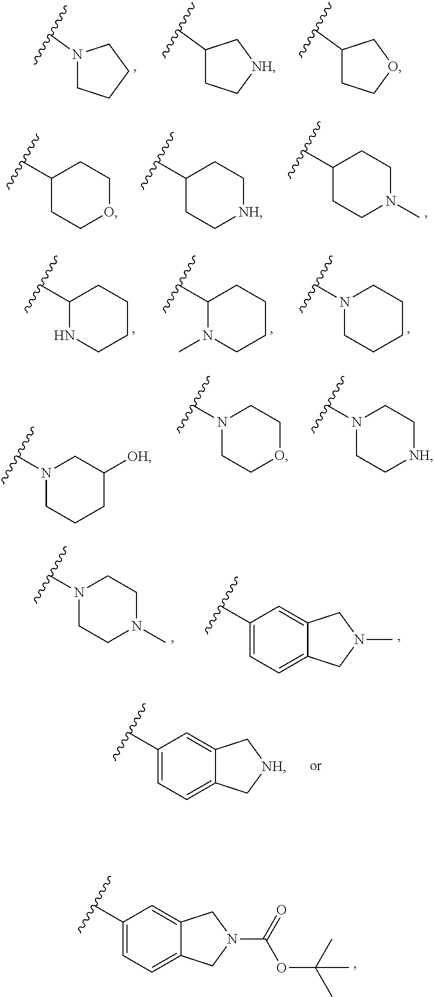

each optionally substituted with 1 or 2 R⁴. In some embodiments, R³ is

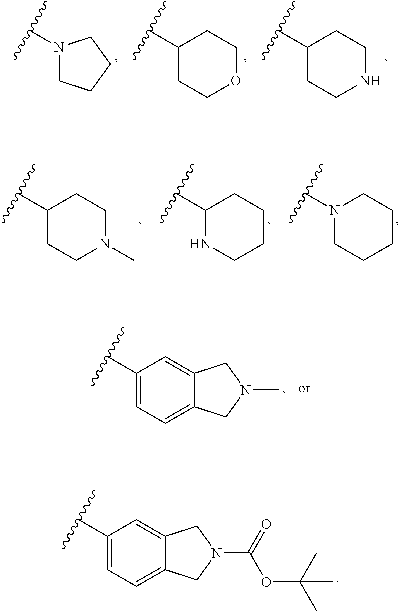

In some embodiments, R³ is

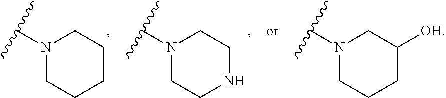

In some embodiments of the compound of formula (I), (I-A), (I-B), (I-C), (I-D), (I-A-1), (I-A-2), (I-A-3), (I-A-4), (I-B-1), (I-B-2), (I-B-3), (I-B-4), (I-C-1), (I-C-2), (I-C-3), (I-C-4), (I-D-1), (I-D-2), (I-D-3), (I-D-4), (I-A-1-a), (I-A-2-a), (I-A-3-a), (I-B-1-a), (I-B-2-a), (I-B-3-a), (I-C-1-a), (I-D-1-a), (II), (II-A), (II-B), (II-C), (II-D), (II-A-1), (II-A-2), (II-A-3), (II-A-4), (II-B3-1), (II-B3-2), (II-B3-3), (II-B3-4), (II-C-1), (II-C-2), (II-C-3), (II-C-4), (II-D-1), (II-D-2), (II-D-3), (II-D-4), (III), (III-A), (III-B), (III-C), (III-D), (III-A-1), (III-A-2), (III-A-3), (III-A-4), (III-B-1), (III-B-2), (III-B-3), (III-B-4), (III-C-1), (III-C-2), (III-C-3), (III-C-4), (III-D-1), (III-D-2), (III-D-3), or (III-D-4), or a pharmaceutically acceptable salt, stereoisomer, or deuterated form thereof, R³ is —NR^{X}R^{Y}, and wherein R^{X} is H, C₁₋₆ alkyl, C₁₋₆ haloalkyl, or C₁₋₆ hydroxyalkyl, and R is C₁₋₆ alkyl, C₃₋₈ cycloalkyl, C₃₋₈ hydroxycycloalkyl, aryl, or heterocycle. In some embodiments, R^{X} is H, or methyl, and R^{Y} is methyl, —CH(CH₃)₂,

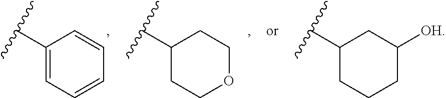

In some embodiments, R³ is

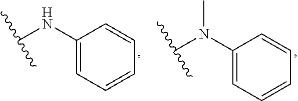

—NHCH(CH₃)₂, —N(CH₃)₂, —NH(CH₃),

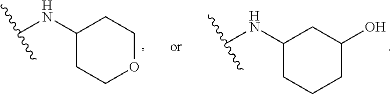 [SMILES: *NC1CCOCC1 ; *NC1CCCC(O)C1]

### -Q-R³

In some embodiments of the compound of formula (I), (I-A), (I-B), (I-C), (I-D), (I-A-1), (I-A-2), (I-A-3), (I-A-4), (I-B-1), (I-B-2), (I-B-3), (I-B-4), (I-C-1), (I-C-2), (I-C-3), (I-C-4), (I-D-1), (I-D-2), (I-D-3), (I-D-4), (I-A-1-a), (I-A-2-a), (I-A-3-a), (I-B-1-a), (I-B-2-a), (I-B-3-a), (I-C-1-a), (I-D-1-a), (II), (II-A), (II-B), (II-C), (II-D), (II-A-1), (II-A-2), (II-A-3), (II-A-4), (II-B3-1), (II-B3-2), (II-B3-3), (II-B3-4), (II-C-1), (II-C-2), (II-C-3), (II-C-4), (II-D-1), (II-D-2), (II-D-3), (II-D-4), (III), (III-A), (III-B), (III-C), (III-D), (III-A-1), (III-A-2), (III-A-3), (III-A-4), (III-B-1), (III-B-2), (III-B-3), (III-B-4), (III-C-1), (III-C-2), (III-C-3), (III-C-4), (III-D-1), (III-D-2), (III-D-3), or (III-D-4), or a pharmaceutically acceptable salt, stereoisomer, or deuterated form thereof, Q is absent, and R³ is

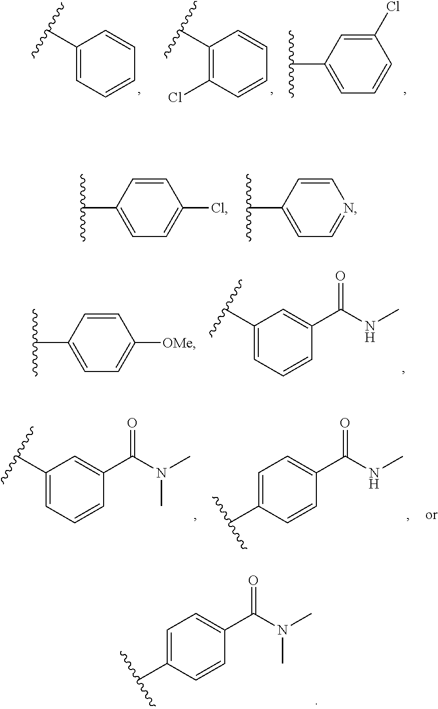

In some embodiments of the compound of formula (I), (I-A), (I-B), (I-C), (I-D), (I-A-1), (I-A-2), (I-A-3), (I-A-4), (I-B-1), (I-B-2), (I-B-3), (I-B-4), (I-C-1), (I-C-2), (I-C-3), (I-C-4), (I-D-1), (I-D-2), (I-D-3), (I-D-4), (I-A-1-a), (I-A-2-a), (I-A-3-a), (I-B-1-a), (I-B-2-a), (I-B-3-a), (I-C-1-a), (I-D-1-a), (II), (II-A), (II-B), (II-C), (II-D), (II-A-1), (II-A-2), (II-A-3), (II-A-4), (II-B3-1), (II-B3-2), (II-B3-3), (II-B3-4), (II-C-1), (II-C-2), (II-C-3), (II-C-4), (II-D-1), (II-D-2), (II-D-3), (II-D-4), (III), (III-A), (III-B), (III-C), (III-D), (III-A-1), (III-A-2), (III-A-3), (III-A-4), (III-B-1), (III-B-2), (III-B-3), (III-B-4), (III-C-1), (III-C-2), (III-C-3), (III-C-4), (III-D-1), (III-D-2), (III-D-3), or (III-D-4), or a pharmaceutically acceptable salt, stereoisomer, or deuterated form thereof, -Q-R³ is

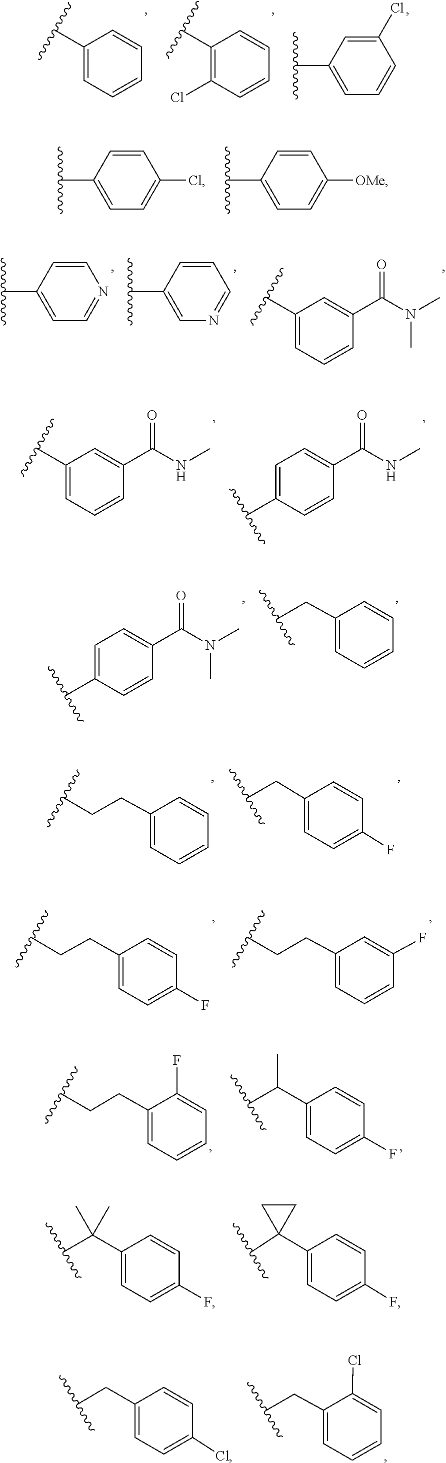

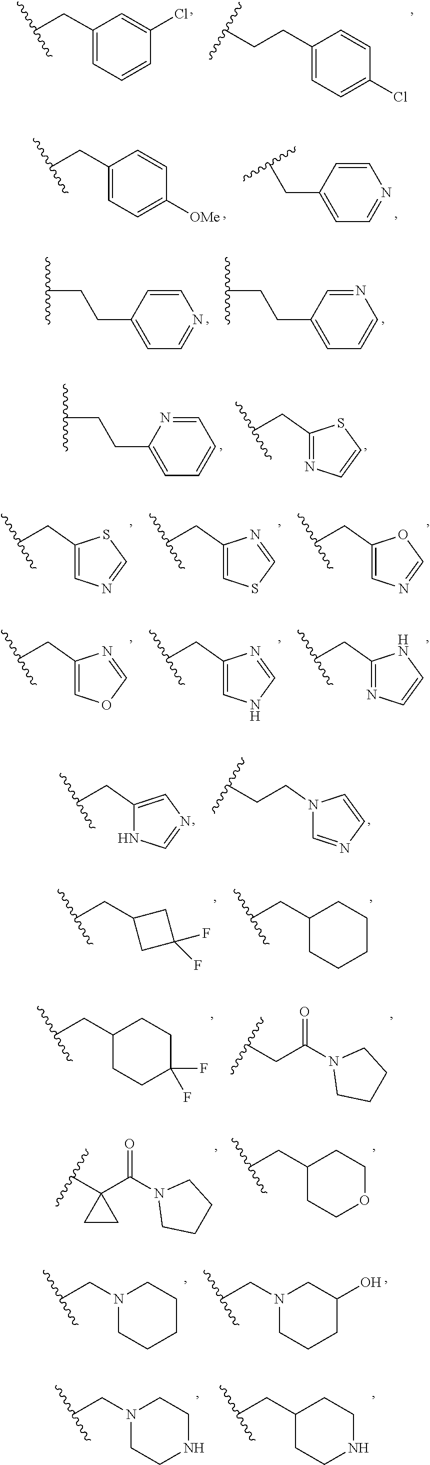

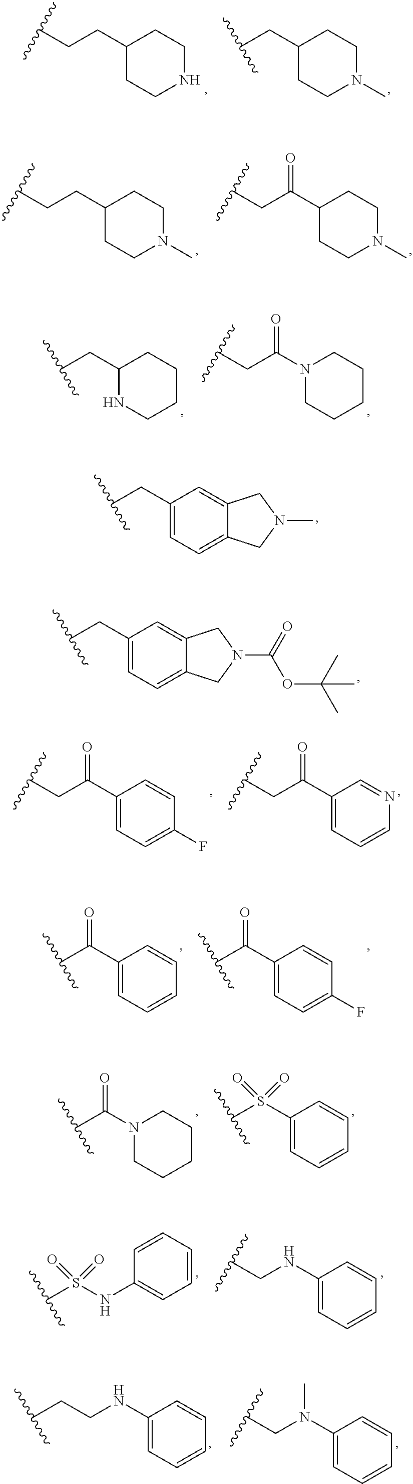

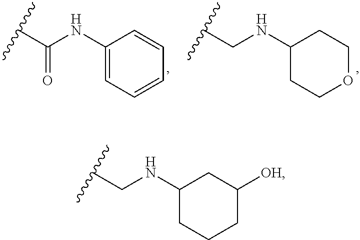

—CH₂C(O)—N(CH₃)₂, —CH₂C(O)—NH(CH₃),

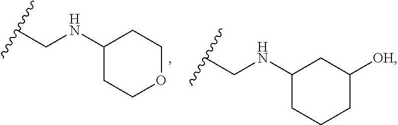

or —CH₂CH₂—NH—CH(CH₃)₂.

In some embodiments of the compound of formula (I), (I-A), (I-B), (I-C), (I-D), (I-A-1), (I-A-2), (I-A-3), (I-A-4), (I-B-1), (I-B-2), (I-B-3), (I-B-4), (I-C-1), (I-C-2), (I-C-3), (I-C-4), (I-D-1), (I-D-2), (I-D-3), (I-D-4), (I-A-1-a), (I-A-2-a), (I-A-3-a), (I-B-1-a), (I-B-2-a), (I-B-3-a), (I-C-1-a), (I-D-1-a), (II), (II-A), (II-B), (II-C), (II-D), (II-A-1), (II-A-2), (II-A-3), (II-A-4), (II-B3-1), (II-B3-2), (II-B3-3), (II-B3-4), (II-C-1), (II-C-2), (II-C-3), (II-C-4), (II-D-1), (II-D-2), (II-D-3), (II-D-4), (III), (III-A), (III-B), (III-C), (III-D), (III-A-1), (III-A-2), (III-A-3), (III-A-4), (III-B-1), (III-B-2), (III-B-3), (III-B-4), (III-C-1), (III-C-2), (III-C-3), (III-C-4), (III-D-1), (III-D-2), (III-D-3), or (III-D-4), or a pharmaceutically acceptable salt, stereoisomer, or deuterated form thereof, -Q-R³ is

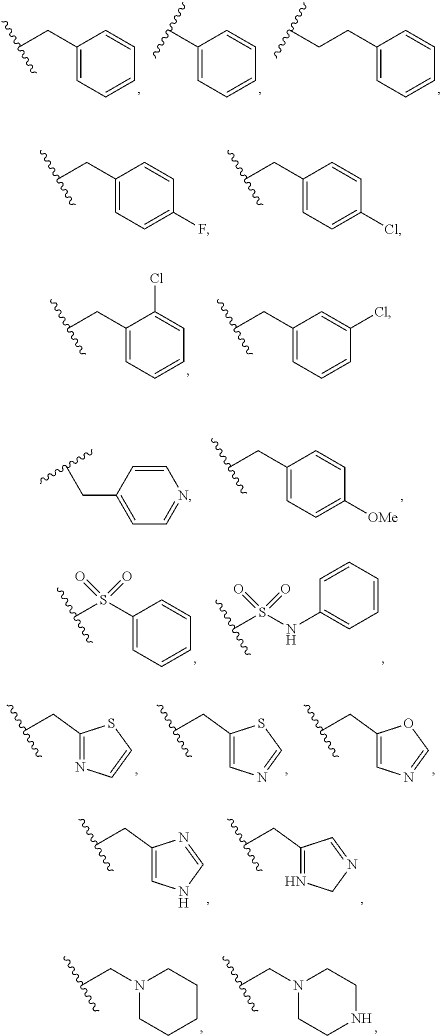

CH₂CH₂—NH—CH(CH₃)₂,

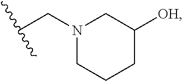 [SMILES: *CN1CCCC(O)C1]

—CH₂C(O)—N(CH₃)₂—CH₂C(O)—NH(CH₃),

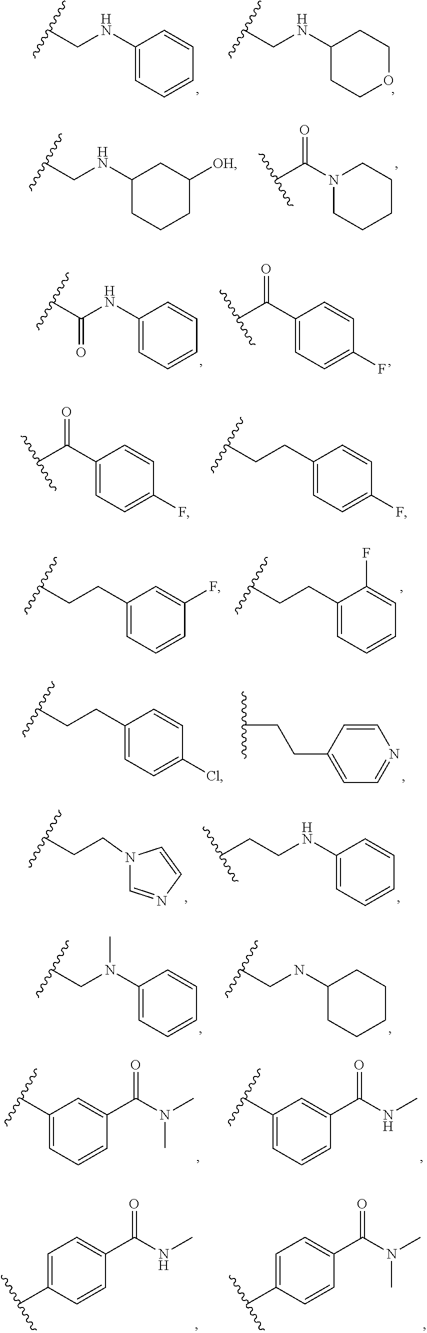

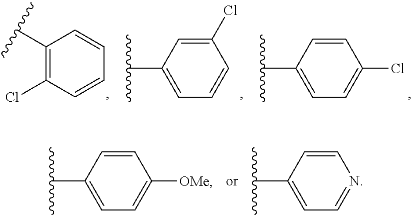

In some embodiments, -Q-R³ is

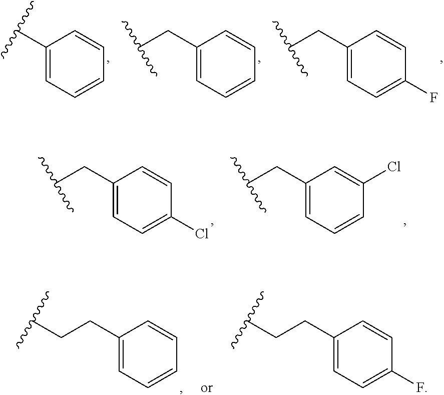

In embodiments, the compounds disclosed herein (e.g., compounds of formula (I), (I-A), (I-B), (I-C), (I-D), (I-A-1), (I-A-2), (I-A-3), (I-A-4), (I-B-1), (I-B-2), (I-B-3), (I-B-4), (I-C-1), (I-C-2), (I-C-3), (I-C-4), (I-D-1), (I-D-2), (I-D-3), (I-D-4), (I-A-1-a), (I-A-2-a), (I-A-3-a), (I-B-1-a), (I-B-2-a), (I-B-3-a), (I-C-1-a), (I-D-1-a), (II), (II-A), (II-B), (II-C), (II-D), (II-A-1), (II-A-2), (II-A-3), (II-A-4), (II-B3-1), (II-B3-2), (II-B3-3), (II-B3-4), (II-C-1), (II-C-2), (II-C-3), (II-C-4), (II-D-1), (II-D-2), (II-D-3), (II-D-4), (III), (III-A), (III-B), (III-C), (III-D), (III-A-1), (III-A-2), (III-A-3), (III-A-4), (III-B-1), (III-B-2), (III-B-3), (III-B-4), (III-C-1), (III-C-2), (III-C-3), (III-C-4), (III-D-1), (III-D-2), (III-D-3), or (III-D-4)), or a pharmaceutically acceptable salt, stereoisomer, or deuterated form thereof) have a molecular weight of up to 1,500 Daltons (Da), up to 1,200 Da, up to 1,000 Da, up to 900 Da, up to 800 Da, up to 700 Da, up to 600 Da, or up to 500 Da. In some embodiments, the compounds disclosed herein have a molecular weight ranging from about 100 Da to about 2,000 Da, from about 200 Da to about 1,800 Da, from about 400 Da to about 1,600 Da, from about 600 Da to about 1,200 Da, from about 700 Da to about 1,000 Da, or from about 800 Da to about 900 Da.

Another embodiment is a product obtainable by any of the processes or examples disclosed herein.

In embodiments, provided herein is a compound of formula (I), (I-A), (I-B), (I-C), (I-D), (I-A-1), (I-A-2), (I-A-3), (I-A-4), (I-B-1), (I-B-2), (I-B-3), (I-B-4), (I-C-1), (I-C-2), (I-C-3), (I-C-4), (I-D-1), (I-D-2), (I-D-3), (I-D-4), (I-A-1-a), (I-A-2-a), (I-A-3-a), (I-B-1-a), (I-B-2-a), (I-B-3-a), (I-C-1-a), (I-D-1-a), (II), (II-A), (II-B), (II-C), (II-D), (II-A-1), (II-A-2), (II-A-3), (II-A-4), (II-B3-1), (II-B3-2), (II-B3-3), (II-B3-4), (II-C-1), (II-C-2), (II-C-3), (II-C-4), (II-D-1), (II-D-2), (II-D-3), (II-D-4), (III), (III-A), (III-B), (III-C), (III-D), (III-A-1), (III-A-2), (III-A-3), (III-A-4), (III-B3-1), (III-B-2), (III-B-3), (III-B-4), (III-C-1), (III-C-2), (III-C-3), (III-C-4), (III-D-1), (III-D-2), (III-D-3), or (III-D-4), or a stereoisomer thereof, or a pharmaceutically acceptable salt thereof.

In embodiments, provided herein is a stereoisomer of a compound of formula (I), (I-A), (I-B), (I-C), (I-D), (I-A-1), (I-A-2), (I-A-3), (I-A-4), (I-B-1), (I-B-2), (I-B-3), (I-B-4), (I-C-1), (I-C-2), (I-C-3), (I-C-4), (I-D-1), (I-D-2), (I-D-3), (I-D-4), (I-A-1-a), (I-A-2-a), (I-A-3-a), (I-B-1-a), (I-B-2-a), (I-B-3-a), (I-C-1-a), (I-D-1-a), (II), (II-A), (II-B), (II-C), (II-D), (II-A-1), (II-A-2), (II-A-3), (II-A-4), (II-B3-1), (II-B3-2), (II-B3-3), (II-B3-4), (II-C-1), (II-C-2), (II-C-3), (II-C-4), (II-D-1), (II-D-2), (II-D-3), (II-D-4), (III), (III-A), (III-B), (III-C), (III-D), (III-A-1), (III-A-2), (III-A-3), (III-A-4), (III-B3-1), (III-B-2), (III-B-3), (III-B-4), (III-C-1), (III-C-2), (III-C-3), (III-C-4), (III-D-1), (III-D-2), (III-D-3), or (III-D-4). In embodiments, provided herein is a pharmaceutically acceptable salt of a compound of formula (I), (I-A), (I-B), (I-C), (I-D), (I-A-1), (I-A-2), (I-A-3), (I-A-4), (I-B-1), (I-B-2), (I-B-3), (I-B-4), (I-C-1), (I-C-2), (I-C-3), (I-C-4), (I-D-1), (I-D-2), (I-D-3), (I-D-4), (I-A-1-a), (I-A-2-a), (I-A-3-a), (I-B-1-a), (I-B-2-a), (I-B-3-a), (I-C-1-a), (I-D-1-a), (II), (II-A), (II-B), (II-C), (II-D), (II-A-1), (II-A-2), (II-A-3), (II-A-4), (II-B3-1), (II-B3-2), (II-B3-3), (II-B3-4), (II-C-1), (II-C-2), (II-C-3), (II-C-4), (II-D-1), (II-D-2), (II-D-3), (II-D-4), (III), (III-A), (III-B), (III-C), (III-D), (III-A-1), (III-A-2), (III-A-3), (III-A-4), (III-B-1), (III-B-2), (III-B-3), (III-B-4), (III-C-1), (III-C-2), (III-C-3), (III-C-4), (III-D-1), (III-D-2), (III-D-3), or (III-D-4). Further embodiments of the disclosure relate to a deuterated compound of formula (I), (I-A), (I-B), (I-C), (I-D), (I-A-1), (I-A-2), (I-A-3), (I-A-4), (I-B-1), (I-B-2), (I-B-3), (I-B-4), (I-C-1), (I-C-2), (I-C-3), (I-C-4), (I-D-1), (I-D-2), (I-D-3), (I-D-4), (I-A-1-a), (I-A-2-a), (I-A-3-a), (I-B-1-a), (I-B-2-a), (I-B-3-a), (I-C-1-a), (I-D-1-a), (II), (II-A), (II-B), (II-C), (II-D), (II-A-1), (II-A-2), (II-A-3), (II-A-4), (II-B3-1), (II-B3-2), (II-B3-3), (II-B3-4), (II-C-1), (II-C-2), (II-C-3), (II-C-4), (II-D-1), (II-D-2), (II-D-3), (II-D-4), (III), (III-A), (III-B), (III-C), (III-D), (III-A-1), (III-A-2), (III-A-3), (III-A-4), (III-B3-1), (III-B-2), (III-B-3), (III-B-4), (III-C-1), (III-C-2), (III-C-3), (III-C-4), (III-D-1), (III-D-2), (III-D-3), or (III-D-4), or a pharmaceutically acceptable salt thereof.

The compound disclosed herein is not

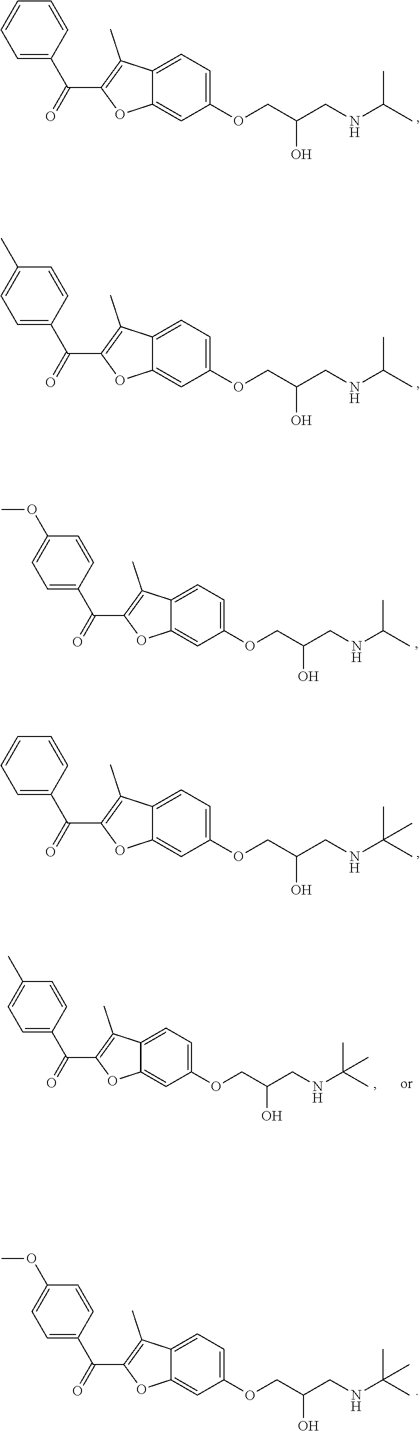

In embodiments, provided herein is a compound in Table 1, 2, or 3, or a pharmaceutically acceptable salt thereof, racemic form thereof, or stereoisomer thereof.

In embodiments, provided herein is a compound in Table 1, 2, or 3, or a pharmaceutically acceptable salt thereof, or stereoisomer thereof.

In embodiments, provided herein is a compound in Table 1, 2, or 3, or a pharmaceutically acceptable salt thereof.

In one embodiment, provided herein is a compound set forth in Table 1, 2, or 3.

In some embodiments, provided herein is a pharmaceutically acceptable salt of a compound in Table 1, 2, or 3.

TABLE 1
Various compounds of the disclosure (series I)

| Comp. | |
|---|---|
| ID | Structure |
| 1 | 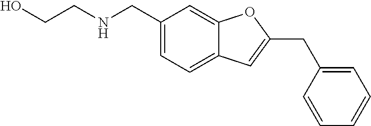 [SMILES: OCCNCc1ccc2cc(Cc3ccccc3)oc2c1] |
| 2 | 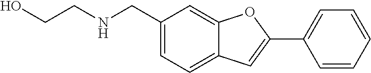 [SMILES: OCCNCc1ccc2cc(-c3ccccc3)oc2c1] |
| 3 | 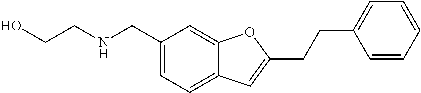 [SMILES: OCCNCc1ccc2cc(CCc3ccccc3)oc2c1] |
| 4 | 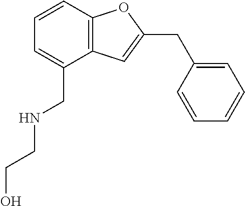 [SMILES: OCCNCc1cccc2oc(Cc3ccccc3)cc12] |
| 5 | 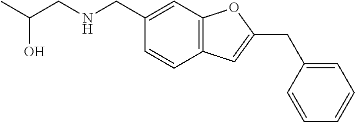 [SMILES: CC(O)CNCc1ccc2cc(Cc3ccccc3)oc2c1] |
| 6 | 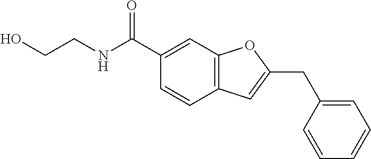 [SMILES: O=C(NCCO)c1ccc2cc(Cc3ccccc3)oc2c1] |
| 7 | 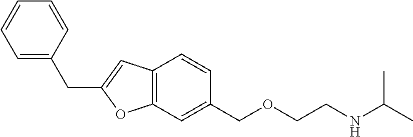 [SMILES: CC(C)NCCOCc1ccc2cc(Cc3ccccc3)oc2c1] |
| 8 | 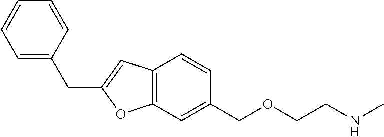 [SMILES: CNCCOCc1ccc2cc(Cc3ccccc3)oc2c1] |
| 9 | 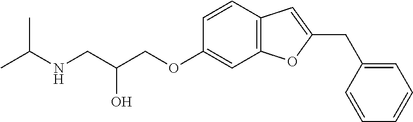 [SMILES: CC(C)NCC(O)COc1ccc2cc(Cc3ccccc3)oc2c1] |
| 10 | 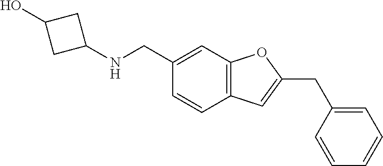 [SMILES: OC1CC(NCc2ccc3cc(Cc4ccccc4)oc3c2)C1] |
| 11 | 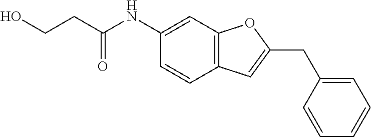 [SMILES: O=C(CCO)Nc1ccc2cc(Cc3ccccc3)oc2c1] |
| 12 | 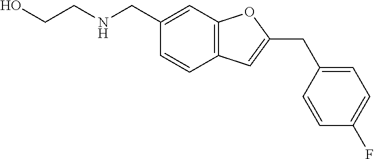 [SMILES: OCCNCc1ccc2cc(Cc3ccc(F)cc3)oc2c1] |
| 13 | 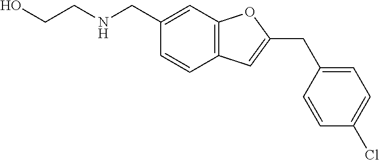 [SMILES: OCCNCc1ccc2cc(Cc3ccc(Cl)cc3)oc2c1] |
| 14 | 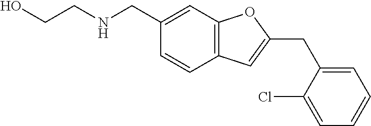 [SMILES: OCCNCc1ccc2cc(Cc3ccccc3Cl)oc2c1] |
| 15 | 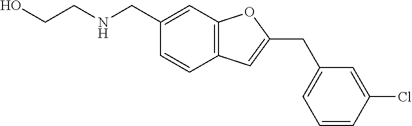 [SMILES: OCCNCc1ccc2cc(Cc3cccc(Cl)c3)oc2c1] |
| 16 | 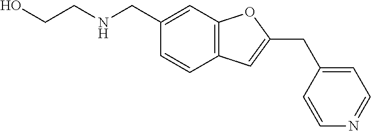 [SMILES: OCCNCc1ccc2cc(Cc3ccncc3)oc2c1] |
| 17 | 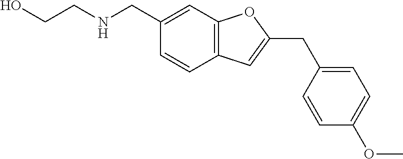 [SMILES: COc1ccc(Cc2cc3ccc(CNCCO)cc3o2)cc1] |
| 18 | 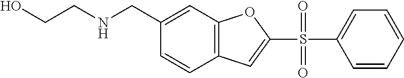 [SMILES: O=S(=O)(c1ccccc1)c1cc2ccc(CNCCO)cc2o1] |
| 19 | 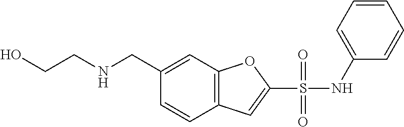 [SMILES: O=S(=O)(Nc1ccccc1)c1cc2ccc(CNCCO)cc2o1] |
| 20 | 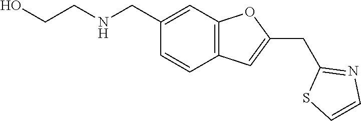 [SMILES: OCCNCc1ccc2cc(Cc3nccs3)oc2c1] |
| 21 | 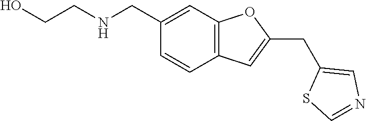 [SMILES: OCCNCc1ccc2cc(Cc3cncs3)oc2c1] |
| 22 | 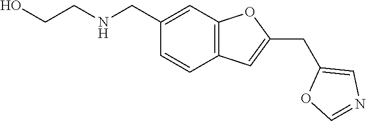 [SMILES: OCCNCc1ccc2cc(Cc3cnco3)oc2c1] |
| 23 | 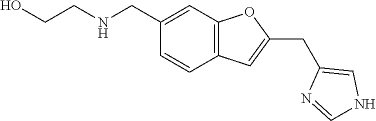 [SMILES: OCCNCc1ccc2cc(Cc3c[nH]cn3)oc2c1] |
| 24 | 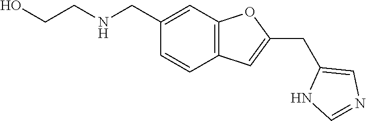 [SMILES: OCCNCc1ccc2cc(Cc3cnc[nH]3)oc2c1] |
| 25 | 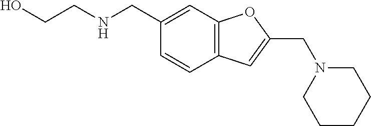 [SMILES: OCCNCc1ccc2cc(CN3CCCCC3)oc2c1] |
| 26 | 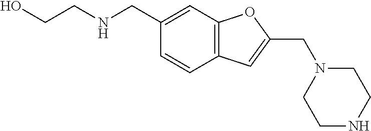 [SMILES: OCCNCc1ccc2cc(CN3CCNCC3)oc2c1] |
| 27 | 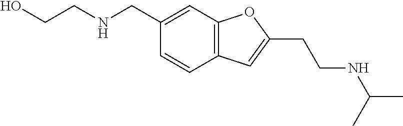 [SMILES: CC(C)NCCc1cc2ccc(CNCCO)cc2o1] |
| 28 | 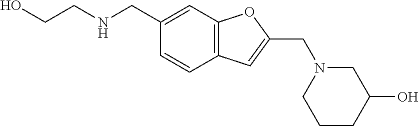 [SMILES: OCCNCc1ccc2cc(CN3CCCC(O)C3)oc2c1] |
| 29 | 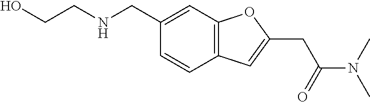 [SMILES: CN(C)C(=O)Cc1cc2ccc(CNCCO)cc2o1] |
| 30 | 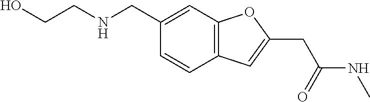 [SMILES: CNC(=O)Cc1cc2ccc(CNCCO)cc2o1] |
| 31 | 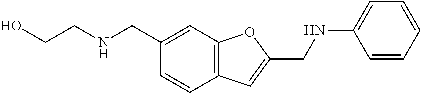 [SMILES: OCCNCc1ccc2cc(CNc3ccccc3)oc2c1] |
| 32 | 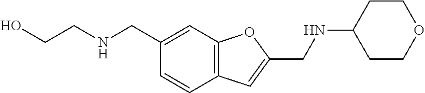 [SMILES: OCCNCc1ccc2cc(CNC3CCOCC3)oc2c1] |
| 33 | 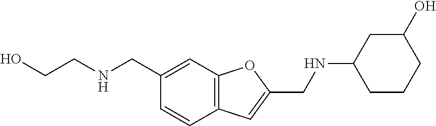 [SMILES: OCCNCc1ccc2cc(CNC3CCCC(O)C3)oc2c1] |
| 34 | 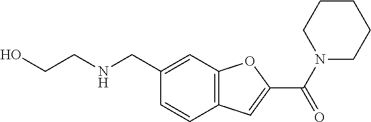 [SMILES: O=C(c1cc2ccc(CNCCO)cc2o1)N1CCCCC1] |
| 35 | 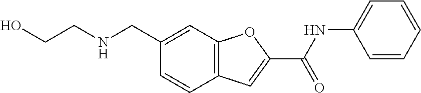 [SMILES: O=C(Nc1ccccc1)c1cc2ccc(CNCCO)cc2o1] |
| 36 | 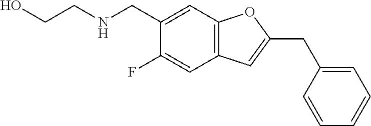 [SMILES: OCCNCc1cc2oc(Cc3ccccc3)cc2cc1F] |
| 37 | 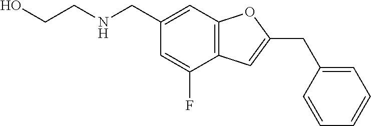 [SMILES: OCCNCc1cc(F)c2cc(Cc3ccccc3)oc2c1] |
| 38 | 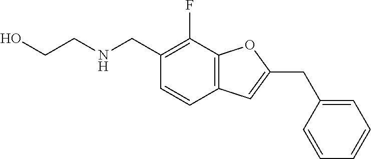 [SMILES: OCCNCc1ccc2cc(Cc3ccccc3)oc2c1F] |
| 39 | 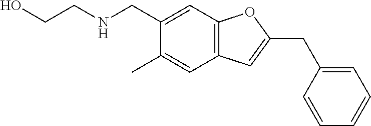 [SMILES: Cc1cc2cc(Cc3ccccc3)oc2cc1CNCCO] |
| 40 | 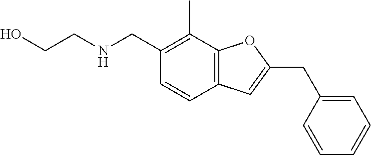 [SMILES: Cc1c(CNCCO)ccc2cc(Cc3ccccc3)oc12] |
| 41 | 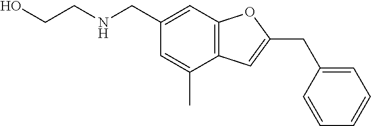 [SMILES: Cc1cc(CNCCO)cc2oc(Cc3ccccc3)cc12] |
| 42 | 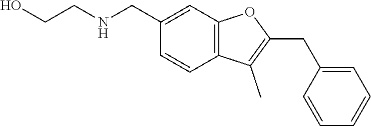 [SMILES: Cc1c(Cc2ccccc2)oc2cc(CNCCO)ccc12] |
| 43 | 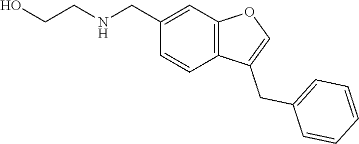 [SMILES: OCCNCc1ccc2c(Cc3ccccc3)coc2c1] |
| 44 | 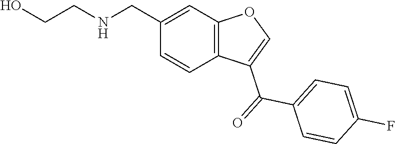 [SMILES: O=C(c1ccc(F)cc1)c1coc2cc(CNCCO)ccc12] |
| 45 | 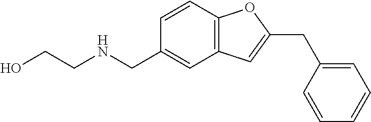 [SMILES: OCCNCc1ccc2oc(Cc3ccccc3)cc2c1] |
| 46 | 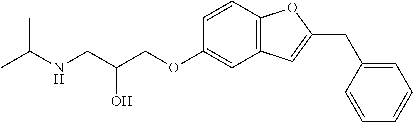 [SMILES: CC(C)NCC(O)COc1ccc2oc(Cc3ccccc3)cc2c1] |
| 47 | 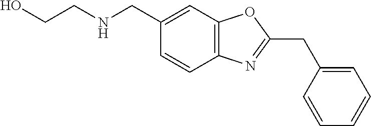 [SMILES: OCCNCc1ccc2nc(Cc3ccccc3)oc2c1] |
| 48 | 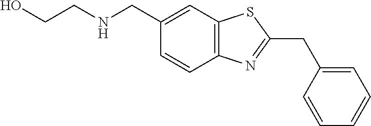 [SMILES: OCCNCc1ccc2nc(Cc3ccccc3)sc2c1] |
| 49 | 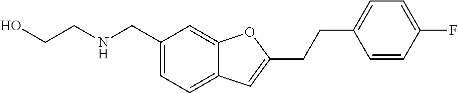 [SMILES: OCCNCc1ccc2cc(CCc3ccc(F)cc3)oc2c1] |
| 50 | 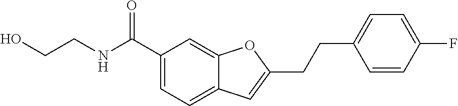 [SMILES: O=C(NCCO)c1ccc2cc(CCc3ccc(F)cc3)oc2c1] |
| 51 | 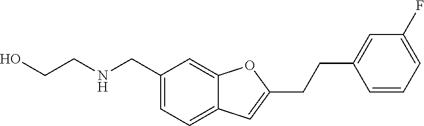 [SMILES: OCCNCc1ccc2cc(CCc3cccc(F)c3)oc2c1] |
| 52 | 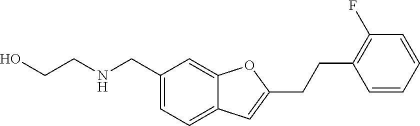 [SMILES: OCCNCc1ccc2cc(CCc3ccccc3F)oc2c1] |
| 53 | 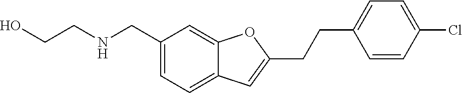 [SMILES: OCCNCc1ccc2cc(CCc3ccc(Cl)cc3)oc2c1] |
| 54 | 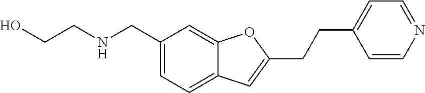 [SMILES: OCCNCc1ccc2cc(CCc3ccncc3)oc2c1] |
| 55 | 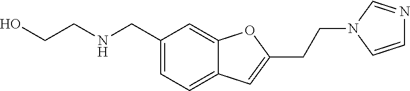 [SMILES: OCCNCc1ccc2cc(CCn3ccnc3)oc2c1] |
| 56 | 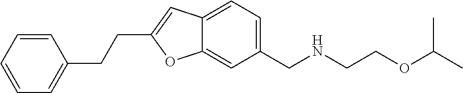 [SMILES: CC(C)OCCNCc1ccc2cc(CCc3ccccc3)oc2c1] |
| 57 | 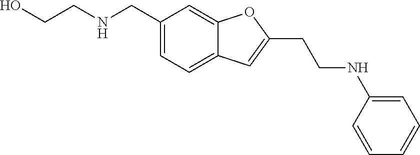 [SMILES: OCCNCc1ccc2cc(CCNc3ccccc3)oc2c1] |
| 58 | 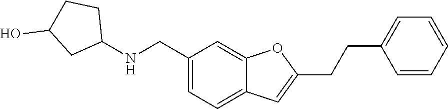 [SMILES: OC1CCC(NCc2ccc3cc(CCc4ccccc4)oc3c2)C1] |
| 59 | 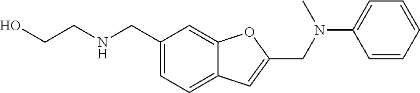 [SMILES: CN(Cc1cc2ccc(CNCCO)cc2o1)c1ccccc1] |
| 60 | 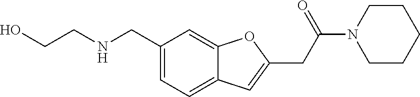 [SMILES: O=C(Cc1cc2ccc(CNCCO)cc2o1)N1CCCCC1] |
| 61 | 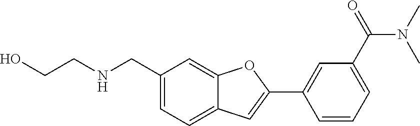 [SMILES: CN(C)C(=O)c1cccc(-c2cc3ccc(CNCCO)cc3o2)c1] |
| 62 | 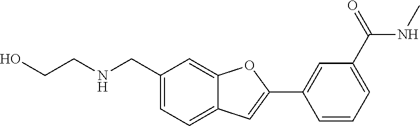 [SMILES: CNC(=O)c1cccc(-c2cc3ccc(CNCCO)cc3o2)c1] |
| 63 | 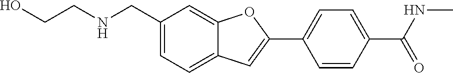 [SMILES: CNC(=O)c1ccc(-c2cc3ccc(CNCCO)cc3o2)cc1] |
| 64 | 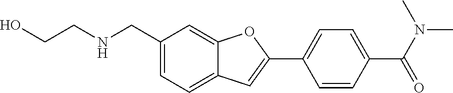 [SMILES: CN(C)C(=O)c1ccc(-c2cc3ccc(CNCCO)cc3o2)cc1] |
| 65 | 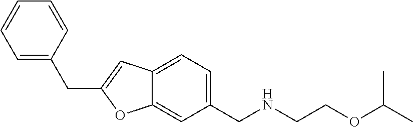 [SMILES: CC(C)OCCNCc1ccc2cc(Cc3ccccc3)oc2c1] |
| 66 | 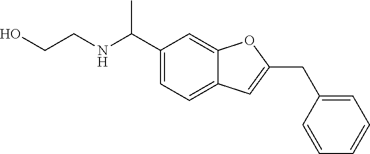 [SMILES: CC(NCCO)c1ccc2cc(Cc3ccccc3)oc2c1] |
| 67 | 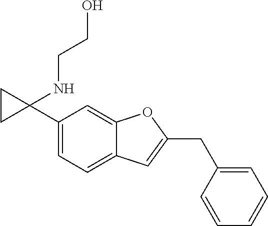 [SMILES: OCCNC1(c2ccc3cc(Cc4ccccc4)oc3c2)CC1] |
| 68 | 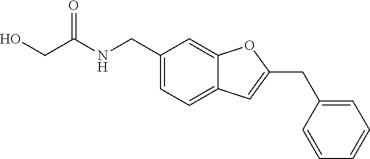 [SMILES: O=C(CO)NCc1ccc2cc(Cc3ccccc3)oc2c1] |
| 69 | 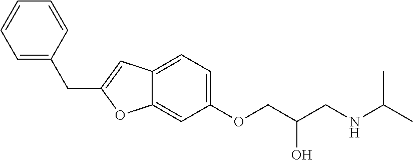 [SMILES: CC(C)NCC(O)COc1ccc2cc(Cc3ccccc3)oc2c1] |
| 70 | 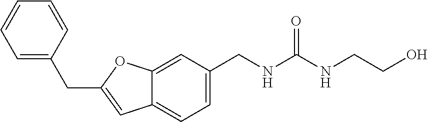 [SMILES: O=C(NCCO)NCc1ccc2cc(Cc3ccccc3)oc2c1] |
| 71 | 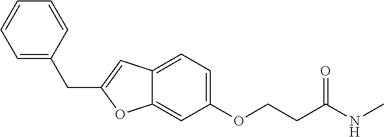 [SMILES: CNC(=O)CCOc1ccc2cc(Cc3ccccc3)oc2c1] |
| 72 | 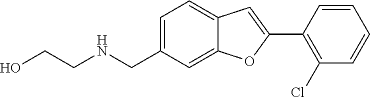 [SMILES: OCCNCc1ccc2cc(-c3ccccc3Cl)oc2c1] |
| 73 | 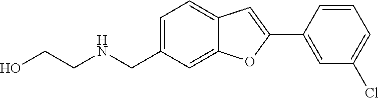 [SMILES: OCCNCc1ccc2cc(-c3cccc(Cl)c3)oc2c1] |
| 74 | 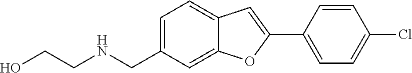 [SMILES: OCCNCc1ccc2cc(-c3ccc(Cl)cc3)oc2c1] |
| 75 | 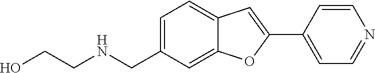 [SMILES: OCCNCc1ccc2cc(-c3ccncc3)oc2c1] |
| 76 | 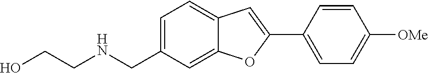 [SMILES: COc1ccc(-c2cc3ccc(CNCCO)cc3o2)cc1] |
| 77 | 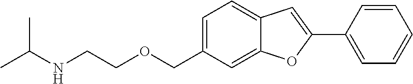 [SMILES: CC(C)NCCOCc1ccc2cc(-c3ccccc3)oc2c1] |
| 78 | 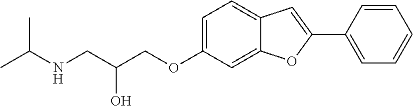 [SMILES: CC(C)NCC(O)COc1ccc2cc(-c3ccccc3)oc2c1] |
| 79 | 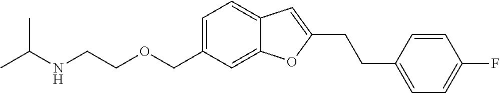 [SMILES: CC(C)NCCOCc1ccc2cc(CCc3ccc(F)cc3)oc2c1] |
| 80 | 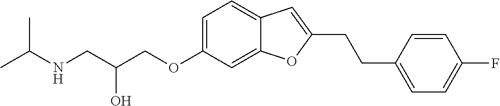 [SMILES: CC(C)NCC(O)COc1ccc2cc(CCc3ccc(F)cc3)oc2c1] |
| 105 | 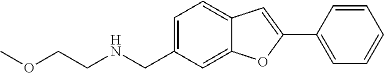 [SMILES: COCCNCc1ccc2cc(-c3ccccc3)oc2c1] |
| 106 | 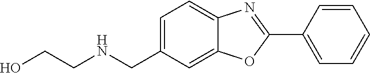 [SMILES: OCCNCc1ccc2nc(-c3ccccc3)oc2c1] |
| 107 | 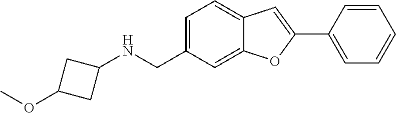 [SMILES: COC1CC(NCc2ccc3cc(-c4ccccc4)oc3c2)C1] |
| 108 | 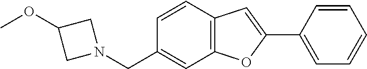 [SMILES: COC1CN(Cc2ccc3cc(-c4ccccc4)oc3c2)C1] |
| 109 | 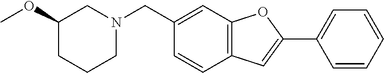 [SMILES: CO[C@@H]1CCCN(Cc2ccc3cc(-c4ccccc4)oc3c2)C1] |
| 110 | 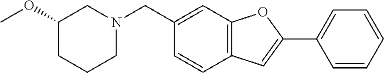 [SMILES: CO[C@H]1CCCN(Cc2ccc3cc(-c4ccccc4)oc3c2)C1] |
| 111 | 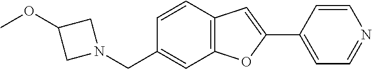 [SMILES: COC1CN(Cc2ccc3cc(-c4ccncc4)oc3c2)C1] |
| 112 | 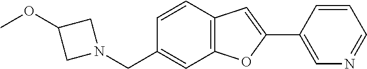 [SMILES: COC1CN(Cc2ccc3cc(-c4cccnc4)oc3c2)C1] |
| 113 | 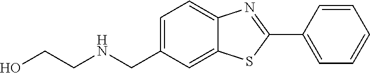 [SMILES: OCCNCc1ccc2nc(-c3ccccc3)sc2c1] |
| 114 | 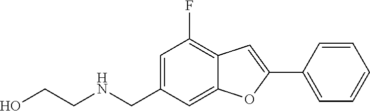 [SMILES: OCCNCc1cc(F)c2cc(-c3ccccc3)oc2c1] |
| 115 | 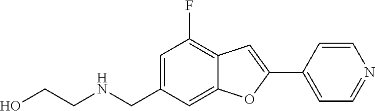 [SMILES: OCCNCc1cc(F)c2cc(-c3ccncc3)oc2c1] |
| 116 | 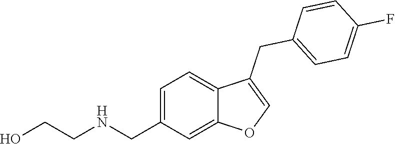 [SMILES: OCCNCc1ccc2c(Cc3ccc(F)cc3)coc2c1] |
| 117 | 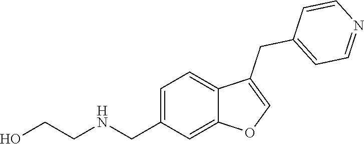 [SMILES: OCCNCc1ccc2c(Cc3ccncc3)coc2c1] |
| 118 | 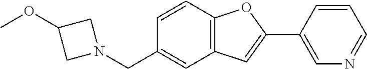 [SMILES: COC1CN(Cc2ccc3oc(-c4cccnc4)cc3c2)C1] |
| 119 | 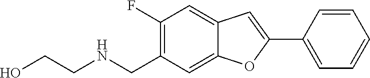 [SMILES: OCCNCc1cc2oc(-c3ccccc3)cc2cc1F] |

TABLE 2
Various compounds of the disclosure (series II)

| Comp. | |
|---|---|
| ID | Structure |
| 81 | 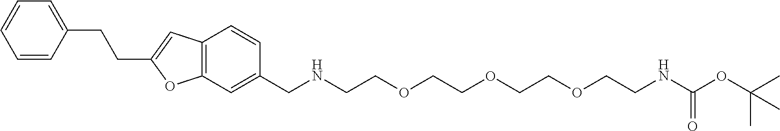 [SMILES: CC(C)(C)OC(=O)NCCOCCOCCOCCNCc1ccc2cc(CCc3ccccc3)oc2c1] |
| 82 | 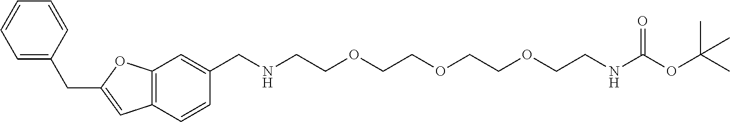 [SMILES: CC(C)(C)OC(=O)NCCOCCOCCOCCNCc1ccc2cc(Cc3ccccc3)oc2c1] |
| 83 | 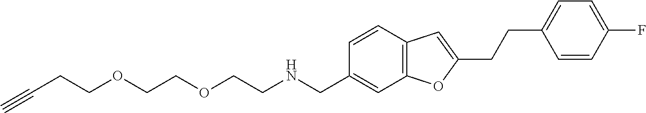 [SMILES: C#CCCOCCOCCNCc1ccc2cc(CCc3ccc(F)cc3)oc2c1] |
| 84 | 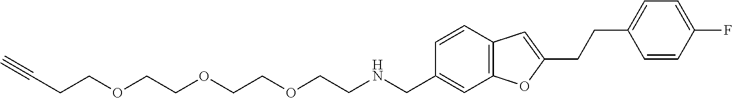 [SMILES: C#CCCOCCOCCOCCNCc1ccc2cc(CCc3ccc(F)cc3)oc2c1] |
| 85 | 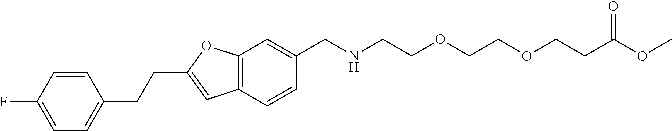 [SMILES: COC(=O)CCOCCOCCNCc1ccc2cc(CCc3ccc(F)cc3)oc2c1] |
| 86 | 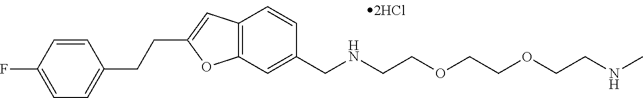 [SMILES: CNCCOCCOCCNCc1ccc2cc(CCc3ccc(F)cc3)oc2c1] |
| 87 | 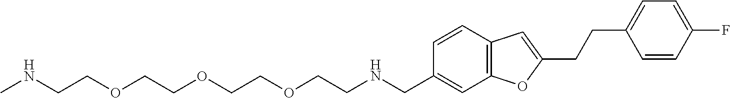 [SMILES: CNCCOCCOCCOCCNCc1ccc2cc(CCc3ccc(F)cc3)oc2c1] |
| 88 | 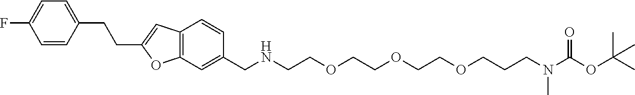 [SMILES: CN(CCCOCCOCCOCCNCc1ccc2cc(CCc3ccc(F)cc3)oc2c1)C(=O)OC(C)(C)C] |
| 89 | 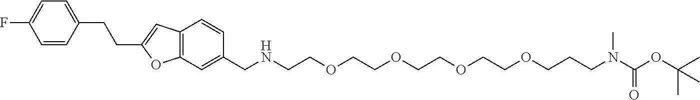 [SMILES: CN(CCCOCCOCCOCCOCCNCc1ccc2cc(CCc3ccc(F)cc3)oc2c1)C(=O)OC(C)(C)C] |
| 90 | 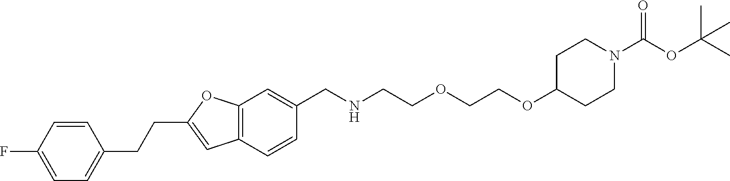 [SMILES: CC(C)(C)OC(=O)N1CCC(OCCOCCNCc2ccc3cc(CCc4ccc(F)cc4)oc3c2)CC1] |
| 91 | 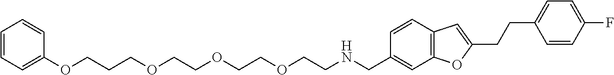 [SMILES: Fc1ccc(CCc2cc3ccc(CNCCOCCOCCOCCCOc4ccccc4)cc3o2)cc1] |
| 92 | 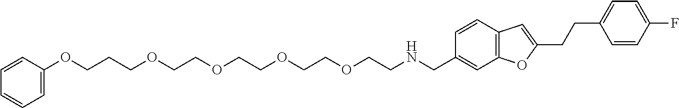 [SMILES: Fc1ccc(CCc2cc3ccc(CNCCOCCOCCOCCOCCCOc4ccccc4)cc3o2)cc1] |
| 93 | 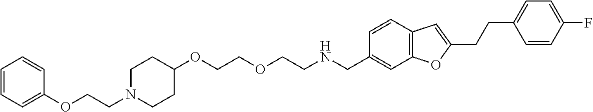 [SMILES: Fc1ccc(CCc2cc3ccc(CNCCOCCOC4CCN(CCOc5ccccc5)CC4)cc3o2)cc1] |
| 94 | 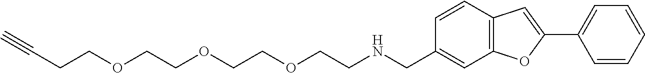 [SMILES: C#CCCOCCOCCOCCNCc1ccc2cc(-c3ccccc3)oc2c1] |
| 95 | 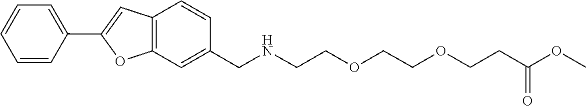 [SMILES: COC(=O)CCOCCOCCNCc1ccc2cc(-c3ccccc3)oc2c1] |
| 96 | 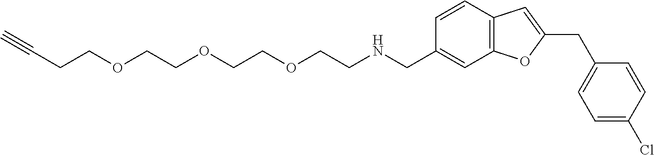 [SMILES: C#CCCOCCOCCOCCNCc1ccc2cc(Cc3ccc(Cl)cc3)oc2c1] |
| 97 | 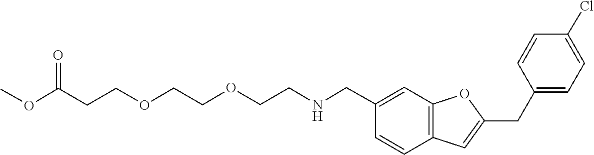 [SMILES: COC(=O)CCOCCOCCNCc1ccc2cc(Cc3ccc(Cl)cc3)oc2c1] |
| 98 | 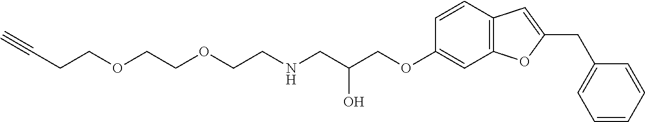 [SMILES: C#CCCOCCOCCNCC(O)COc1ccc2cc(Cc3ccccc3)oc2c1] |
| 99 | 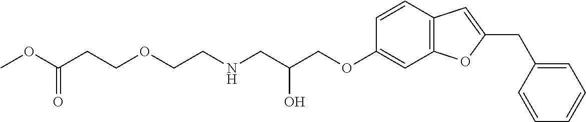 [SMILES: COC(=O)CCOCCNCC(O)COc1ccc2cc(Cc3ccccc3)oc2c1] |
| 120 | 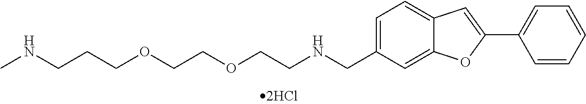 [SMILES: CNCCCOCCOCCNCc1ccc2cc(-c3ccccc3)oc2c1] |
| 121 | 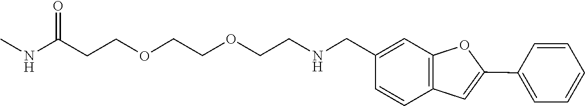 [SMILES: CNC(=O)CCOCCOCCNCc1ccc2cc(-c3ccccc3)oc2c1] |
| 122 | 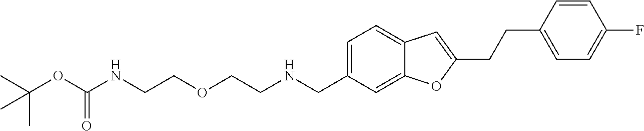 [SMILES: CC(C)(C)OC(=O)NCCOCCNCc1ccc2cc(CCc3ccc(F)cc3)oc2c1] |
| 123 | 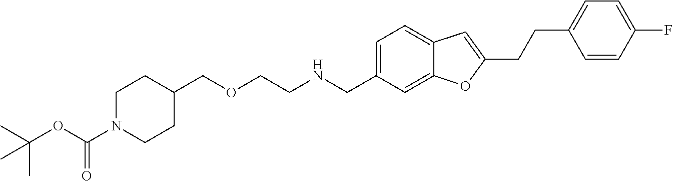 [SMILES: CC(C)(C)OC(=O)N1CCC(COCCNCc2ccc3cc(CCc4ccc(F)cc4)oc3c2)CC1] |
| 124 | 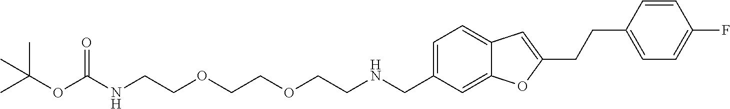 [SMILES: CC(C)(C)OC(=O)NCCOCCOCCNCc1ccc2cc(CCc3ccc(F)cc3)oc2c1] |
| 125 | 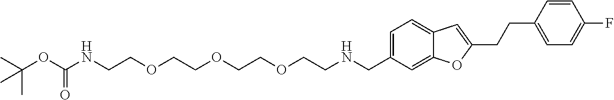 [SMILES: CC(C)(C)OC(=O)NCCOCCOCCOCCNCc1ccc2cc(CCc3ccc(F)cc3)oc2c1] |
| 126 | 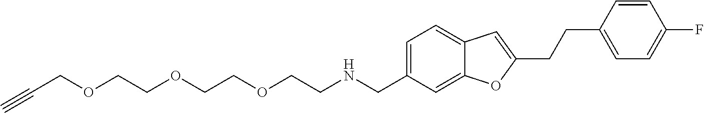 [SMILES: C#CCOCCOCCOCCNCc1ccc2cc(CCc3ccc(F)cc3)oc2c1] |
| 127 | 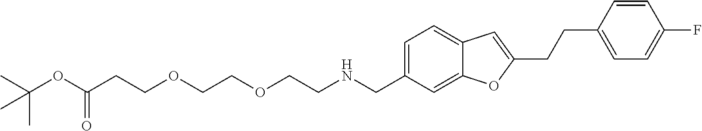 [SMILES: CC(C)(C)OC(=O)CCOCCOCCNCc1ccc2cc(CCc3ccc(F)cc3)oc2c1] |
| 128 | 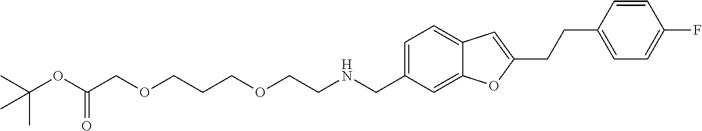 [SMILES: CC(C)(C)OC(=O)COCCCOCCNCc1ccc2cc(CCc3ccc(F)cc3)oc2c1] |
| 129 | 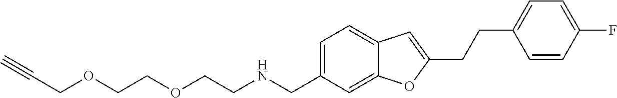 [SMILES: C#CCOCCOCCNCc1ccc2cc(CCc3ccc(F)cc3)oc2c1] |
| 130 | 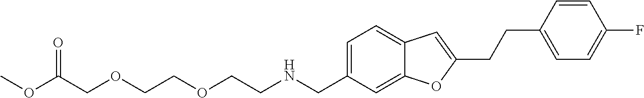 [SMILES: COC(=O)COCCOCCNCc1ccc2cc(CCc3ccc(F)cc3)oc2c1] |
| 131 | 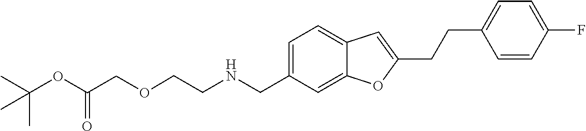 [SMILES: CC(C)(C)OC(=O)COCCNCc1ccc2cc(CCc3ccc(F)cc3)oc2c1] |
| 132 | 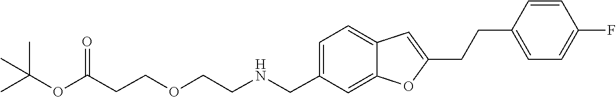 [SMILES: CC(C)(C)OC(=O)CCOCCNCc1ccc2cc(CCc3ccc(F)cc3)oc2c1] |
| 133 | 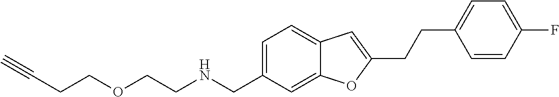 [SMILES: C#CCCOCCNCc1ccc2cc(CCc3ccc(F)cc3)oc2c1] |
| 134 | 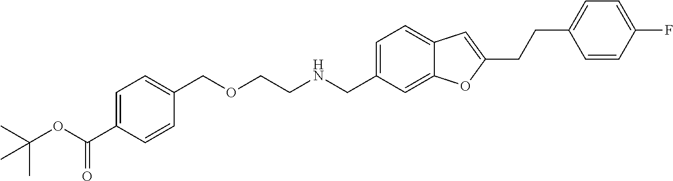 [SMILES: CC(C)(C)OC(=O)c1ccc(COCCNCc2ccc3cc(CCc4ccc(F)cc4)oc3c2)cc1] |
| 135 | 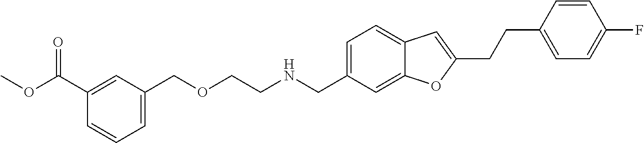 [SMILES: COC(=O)c1cccc(COCCNCc2ccc3cc(CCc4ccc(F)cc4)oc3c2)c1] |
| 136 | 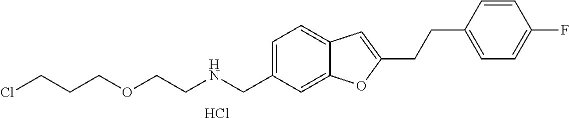 [SMILES: Fc1ccc(CCc2cc3ccc(CNCCOCCCCl)cc3o2)cc1 ; Cl] |
| 137 | 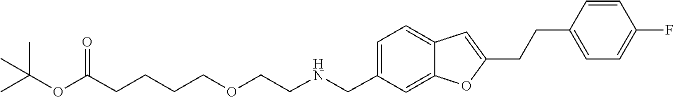 [SMILES: CC(C)(C)OC(=O)CCCCOCCNCc1ccc2cc(CCc3ccc(F)cc3)oc2c1] |
| 138 | 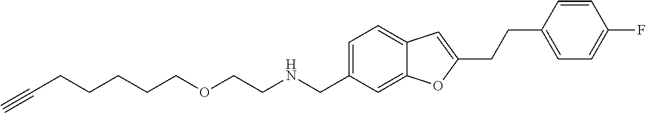 [SMILES: C#CCCCCCOCCNCc1ccc2cc(CCc3ccc(F)cc3)oc2c1] |
| 139 | 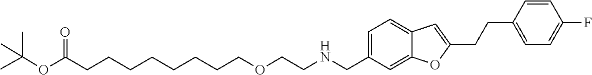 [SMILES: CC(C)(C)OC(=O)CCCCCCCCOCCNCc1ccc2cc(CCc3ccc(F)cc3)oc2c1] |
| 140 | 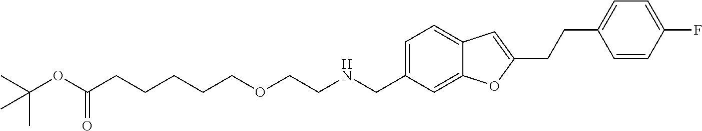 [SMILES: CC(C)(C)OC(=O)CCCCCOCCNCc1ccc2cc(CCc3ccc(F)cc3)oc2c1] |
| 141 | 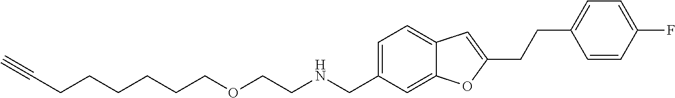 [SMILES: C#CCCCCCCOCCNCc1ccc2cc(CCc3ccc(F)cc3)oc2c1] |
| 142 | 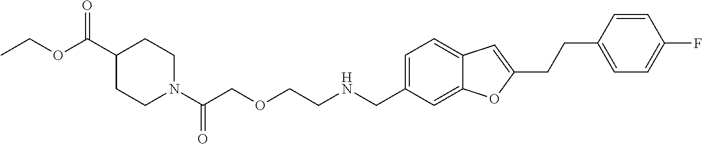 [SMILES: CCOC(=O)C1CCN(C(=O)COCCNCc2ccc3cc(CCc4ccc(F)cc4)oc3c2)CC1] |
| 143 | 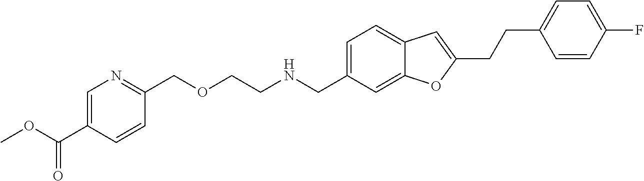 |
| 144 | 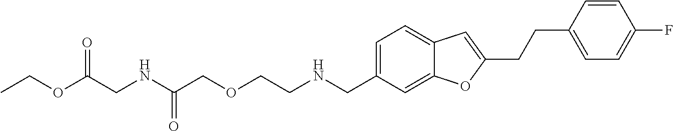 [SMILES: CCOC(=O)CNC(=O)COCCNCc1ccc2cc(CCc3ccc(F)cc3)oc2c1] |
| 145 | 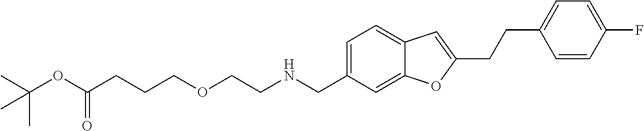 [SMILES: CC(C)(C)OC(=O)CCCOCCNCc1ccc2cc(CCc3ccc(F)cc3)oc2c1] |
| 146 | 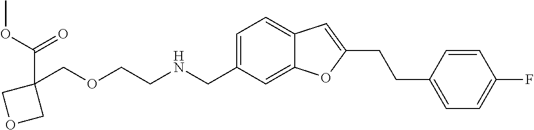 [SMILES: COC(=O)C1(COCCNCc2ccc3cc(CCc4ccc(F)cc4)oc3c2)COC1] |
| 147 | 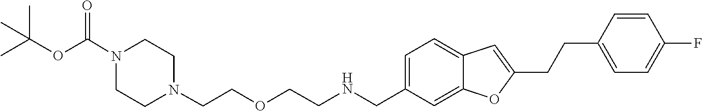 [SMILES: CC(C)(C)OC(=O)N1CCN(CCOCCNCc2ccc3cc(CCc4ccc(F)cc4)oc3c2)CC1] |
| 148 | 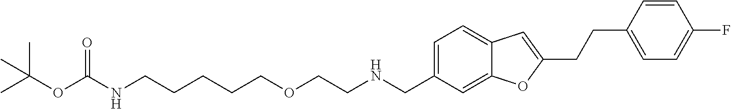 [SMILES: CC(C)(C)OC(=O)NCCCCCOCCNCc1ccc2cc(CCc3ccc(F)cc3)oc2c1] |
| 149 | 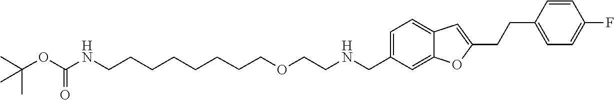 [SMILES: CC(C)(C)OC(=O)NCCCCCCCCOCCNCc1ccc2cc(CCc3ccc(F)cc3)oc2c1] |
| 150 | 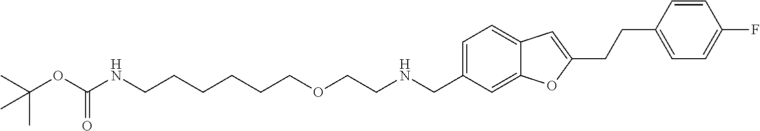 [SMILES: CC(C)(C)OC(=O)NCCCCCCOCCNCc1ccc2cc(CCc3ccc(F)cc3)oc2c1] |
| 151 | 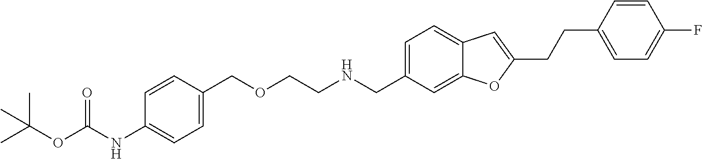 [SMILES: CC(C)(C)OC(=O)Nc1ccc(COCCNCc2ccc3cc(CCc4ccc(F)cc4)oc3c2)cc1] |
| 152 | 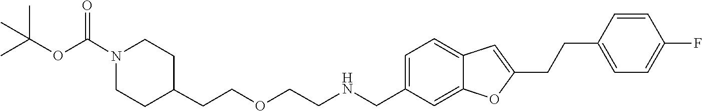 [SMILES: CC(C)(C)OC(=O)N1CCC(CCOCCNCc2ccc3cc(CCc4ccc(F)cc4)oc3c2)CC1] |
| 153 | 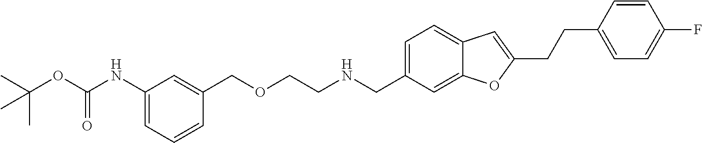 [SMILES: CC(C)(C)OC(=O)Nc1cccc(COCCNCc2ccc3cc(CCc4ccc(F)cc4)oc3c2)c1] |
| 154 | 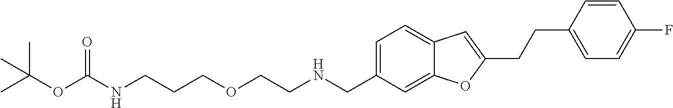 [SMILES: CC(C)(C)OC(=O)NCCCOCCNCc1ccc2cc(CCc3ccc(F)cc3)oc2c1] |
| 155 | 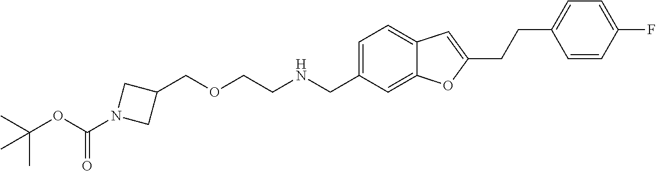 [SMILES: CC(C)(C)OC(=O)N1CC(COCCNCc2ccc3cc(CCc4ccc(F)cc4)oc3c2)C1] |
| 156 | 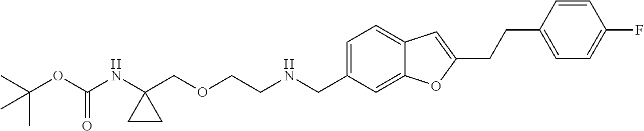 [SMILES: CC(C)(C)OC(=O)NC1(COCCNCc2ccc3cc(CCc4ccc(F)cc4)oc3c2)CC1] |
| 157 | 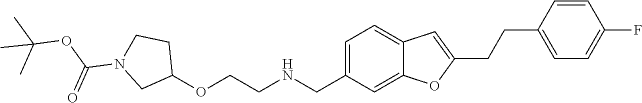 [SMILES: CC(C)(C)OC(=O)N1CCC(OCCNCc2ccc3cc(CCc4ccc(F)cc4)oc3c2)C1] |
| 158 | 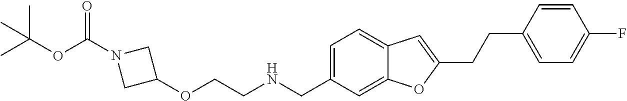 [SMILES: CC(C)(C)OC(=O)N1CC(OCCNCc2ccc3cc(CCc4ccc(F)cc4)oc3c2)C1] |
| 159 | 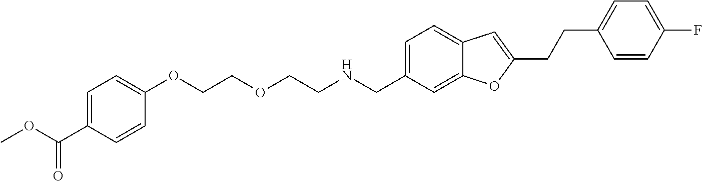 [SMILES: COC(=O)c1ccc(OCCOCCNCc2ccc3cc(CCc4ccc(F)cc4)oc3c2)cc1] |
| 160 | 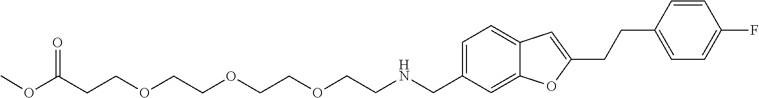 [SMILES: COC(=O)CCOCCOCCOCCNCc1ccc2cc(CCc3ccc(F)cc3)oc2c1] |
| 161 | 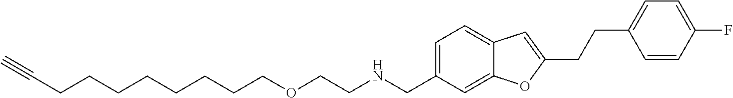 [SMILES: C#CCCCCCCCCOCCNCc1ccc2cc(CCc3ccc(F)cc3)oc2c1] |
| 162 | 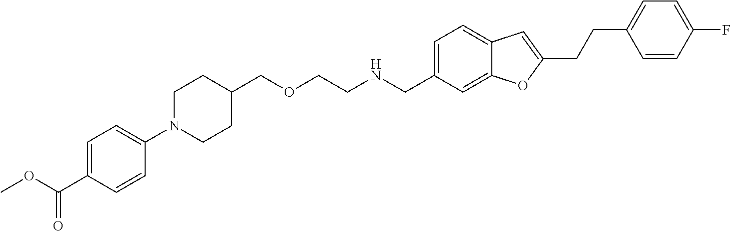 [SMILES: COC(=O)c1ccc(N2CCC(COCCNCc3ccc4cc(CCc5ccc(F)cc5)oc4c3)CC2)cc1] |
| 163 | 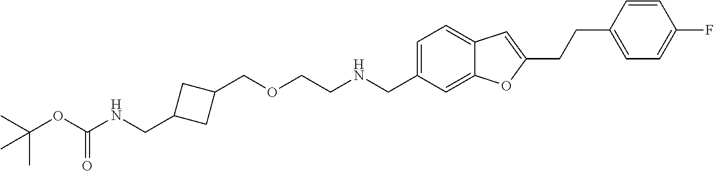 [SMILES: CC(C)(C)OC(=O)NCC1CC(COCCNCc2ccc3cc(CCc4ccc(F)cc4)oc3c2)C1] |
| 164 | 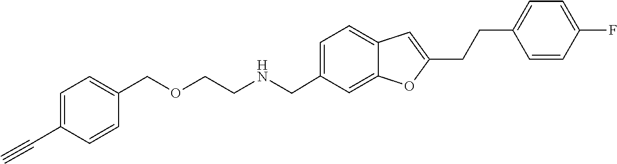 [SMILES: C#Cc1ccc(COCCNCc2ccc3cc(CCc4ccc(F)cc4)oc3c2)cc1] |
| 165 | 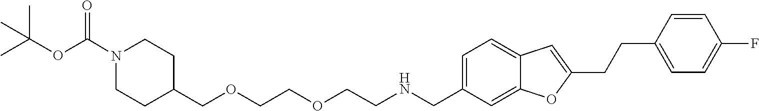 [SMILES: CC(C)(C)OC(=O)N1CCC(COCCOCCNCc2ccc3cc(CCc4ccc(F)cc4)oc3c2)CC1] |
| 166 | 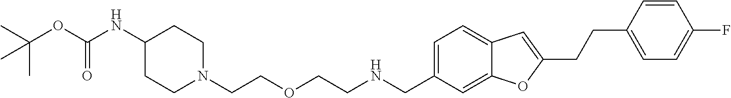 [SMILES: CC(C)(C)OC(=O)NC1CCN(CCOCCNCc2ccc3cc(CCc4ccc(F)cc4)oc3c2)CC1] |
| 167 | 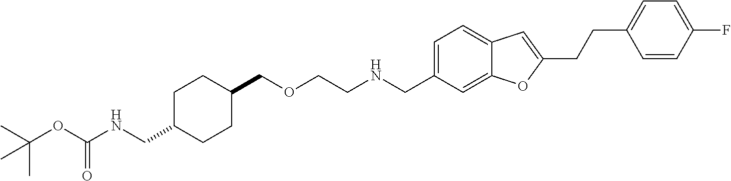 [SMILES: CC(C)(C)OC(=O)NC[C@H]1CC[C@H](COCCNCc2ccc3cc(CCc4ccc(F)cc4)oc3c2)CC1] |
| 168 | 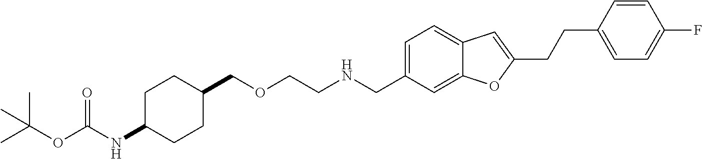 |

TABLE 3
Various compounds of the disclosure (series III)

| Comp. | |
|---|---|
| ID | Structure |
| 100 | 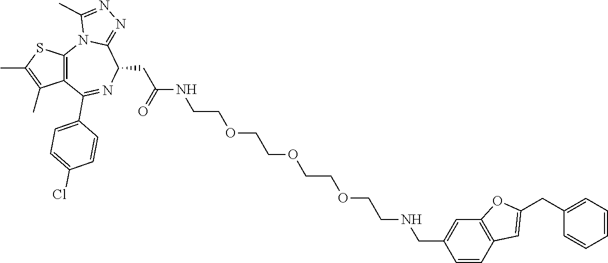 [SMILES: Cc1sc2c(c1C)C(c1ccc(Cl)cc1)=N[C@@H](CC(=O)NCCOCCOCCOCCNCc1ccc3cc(Cc4ccccc4)oc3c1)c1nnc(C)n1-2] |
| 101 | 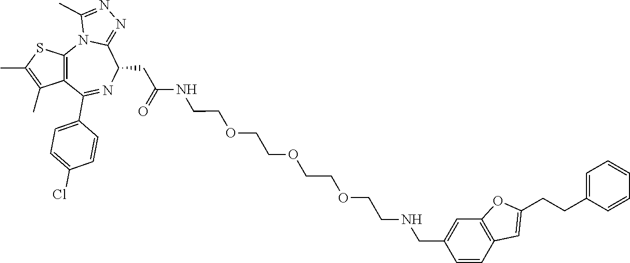 [SMILES: Cc1sc2c(c1C)C(c1ccc(Cl)cc1)=N[C@@H](CC(=O)NCCOCCOCCOCCNCc1ccc3cc(CCc4ccccc4)oc3c1)c1nnc(C)n1-2] |
| 102 | 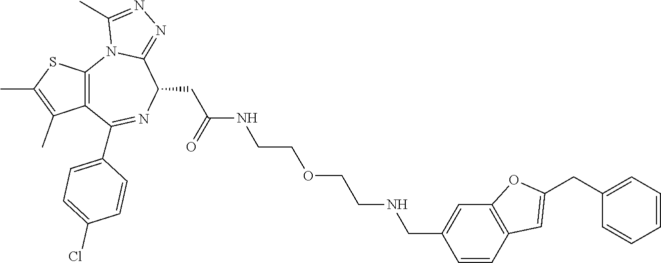 [SMILES: Cc1sc2c(c1C)C(c1ccc(Cl)cc1)=N[C@@H](CC(=O)NCCOCCNCc1ccc3cc(Cc4ccccc4)oc3c1)c1nnc(C)n1-2] |
| 103 | 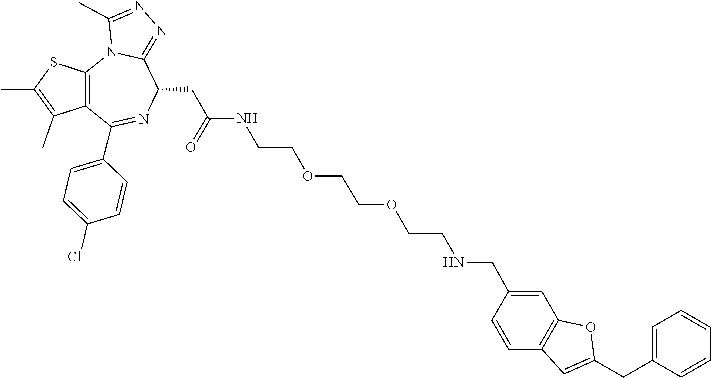 [SMILES: Cc1sc2c(c1C)C(c1ccc(Cl)cc1)=N[C@@H](CC(=O)NCCOCCOCCNCc1ccc3cc(Cc4ccccc4)oc3c1)c1nnc(C)n1-2] |
| 104 | 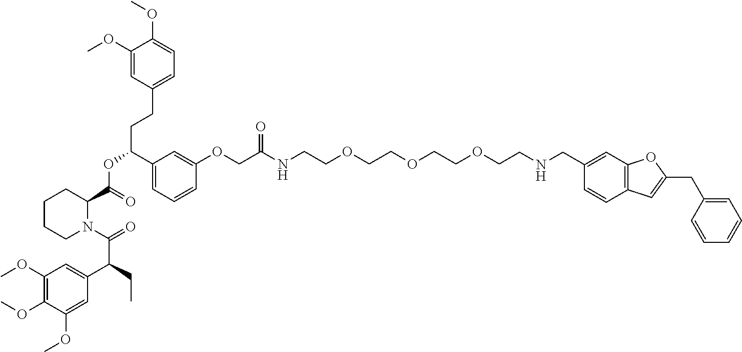 [SMILES: CC[C@H](C(=O)N1CCCC[C@H]1C(=O)O[C@H](CCc1ccc(OC)c(OC)c1)c1cccc(OCC(=O)NCCOCCOCCOCCNCc2ccc3cc(Cc4ccccc4)oc3c2)c1)c1cc(OC)c(OC)c(OC)c1] |
| 169 | 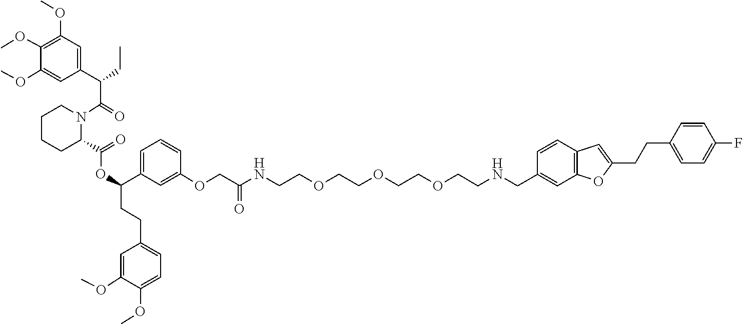 [SMILES: CC[C@H](C(=O)N1CCCC[C@H]1C(=O)O[C@H](CCc1ccc(OC)c(OC)c1)c1cccc(OCC(=O)NCCOCCOCCOCCNCc2ccc3cc(CCc4ccc(F)cc4)oc3c2)c1)c1cc(OC)c(OC)c(OC)c1] |
| 170 | 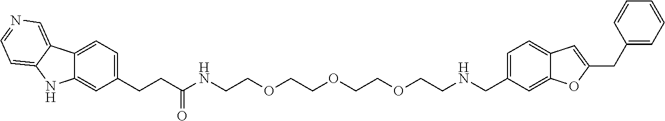 [SMILES: O=C(CCc1ccc2c(c1)[nH]c1ccncc12)NCCOCCOCCOCCNCc1ccc2cc(Cc3ccccc3)oc2c1] |
| 171 | 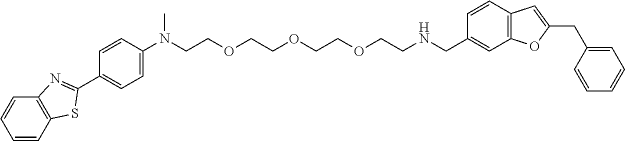 [SMILES: CN(CCOCCOCCOCCNCc1ccc2cc(Cc3ccccc3)oc2c1)c1ccc(-c2nc3ccccc3s2)cc1] |
| 172 | 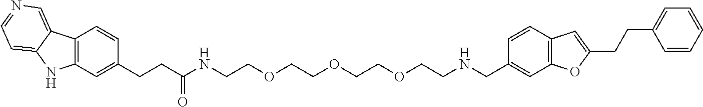 [SMILES: O=C(CCc1ccc2c(c1)[nH]c1ccncc12)NCCOCCOCCOCCNCc1ccc2cc(CCc3ccccc3)oc2c1] |
| 173 | 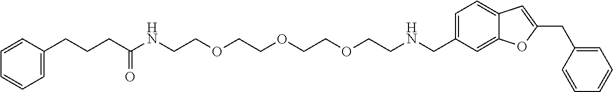 [SMILES: O=C(CCCc1ccccc1)NCCOCCOCCOCCNCc1ccc2cc(Cc3ccccc3)oc2c1] |
| 174 | 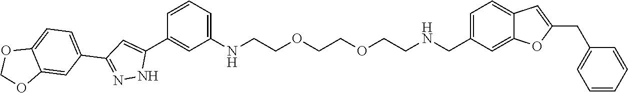 [SMILES: c1ccc(Cc2cc3ccc(CNCCOCCOCCNc4cccc(-c5cc(-c6ccc7c(c6)OCO7)n[nH]5)c4)cc3o2)cc1] |
| 175 | 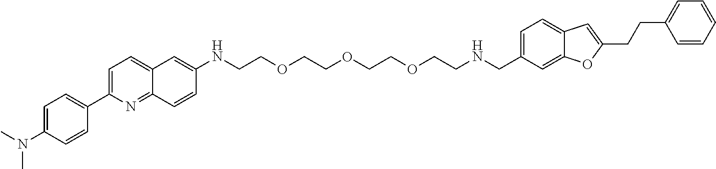 [SMILES: CN(C)c1ccc(-c2ccc3cc(NCCOCCOCCOCCNCc4ccc5cc(CCc6ccccc6)oc5c4)ccc3n2)cc1] |
| 176 | 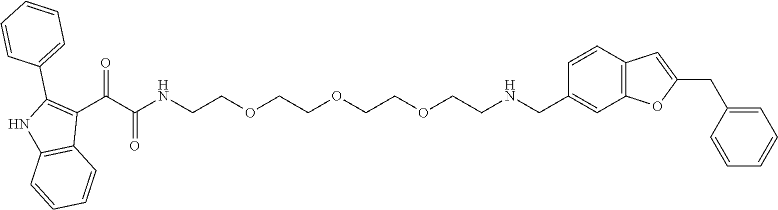 [SMILES: O=C(NCCOCCOCCOCCNCc1ccc2cc(Cc3ccccc3)oc2c1)C(=O)c1c(-c2ccccc2)[nH]c2ccccc12] |
| 177 | 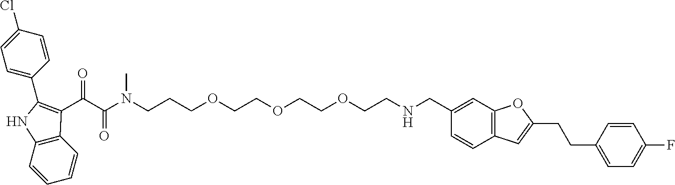 [SMILES: CN(CCCOCCOCCOCCNCc1ccc2cc(CCc3ccc(F)cc3)oc2c1)C(=O)C(=O)c1c(-c2ccc(Cl)cc2)[nH]c2ccccc12] |
| 178 | 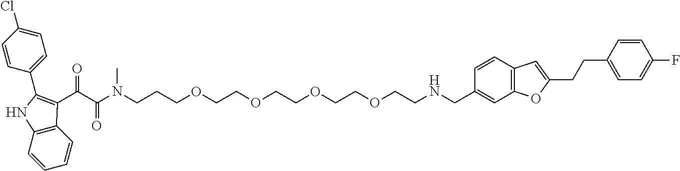 [SMILES: CN(CCCOCCOCCOCCOCCNCc1ccc2cc(CCc3ccc(F)cc3)oc2c1)C(=O)C(=O)c1c(-c2ccc(Cl)cc2)[nH]c2ccccc12] |
| 179 | 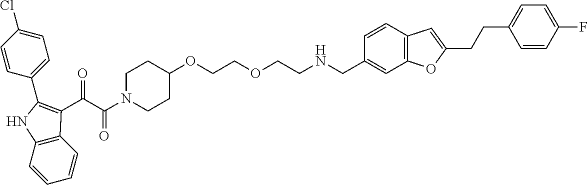 [SMILES: O=C(C(=O)N1CCC(OCCOCCNCc2ccc3cc(CCc4ccc(F)cc4)oc3c2)CC1)c1c(-c2ccc(Cl)cc2)[nH]c2ccccc12] |
| 180 | 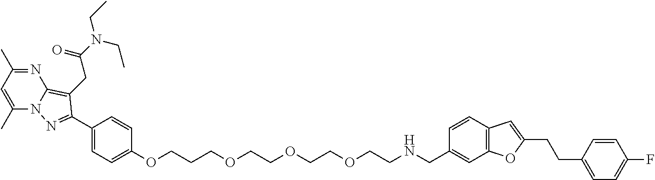 [SMILES: CCN(CC)C(=O)Cc1c(-c2ccc(OCCCOCCOCCOCCNCc3ccc4cc(CCc5ccc(F)cc5)oc4c3)cc2)nn2c(C)cc(C)nc12] |
| 181 | 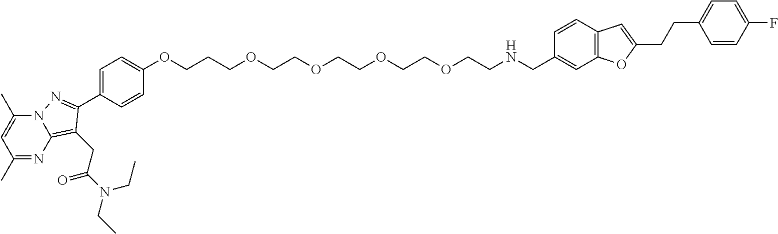 [SMILES: CCN(CC)C(=O)Cc1c(-c2ccc(OCCCOCCOCCOCCOCCNCc3ccc4cc(CCc5ccc(F)cc5)oc4c3)cc2)nn2c(C)cc(C)nc12] |
| 182 | 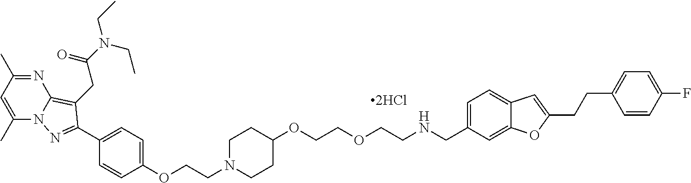 [SMILES: CCN(CC)C(=O)Cc1c(-c2ccc(OCCN3CCC(OCCOCCNCc4ccc5cc(CCc6ccc(F)cc6)oc5c4)CC3)cc2)nn2c(C)cc(C)nc12] |
| 183 | 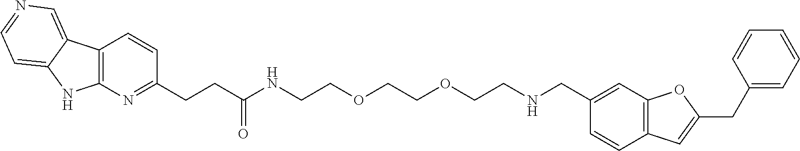 [SMILES: O=C(CCc1ccc2c(n1)[nH]c1ccncc12)NCCOCCOCCNCc1ccc2cc(Cc3ccccc3)oc2c1] |
| 184 | 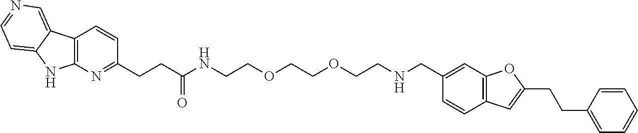 [SMILES: O=C(CCc1ccc2c(n1)[nH]c1ccncc12)NCCOCCOCCNCc1ccc2cc(CCc3ccccc3)oc2c1] |
| 185 | 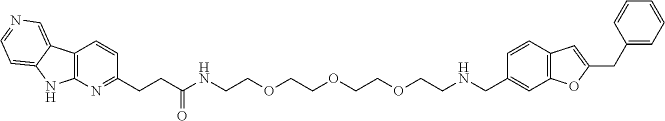 [SMILES: O=C(CCc1ccc2c(n1)[nH]c1ccncc12)NCCOCCOCCOCCNCc1ccc2cc(Cc3ccccc3)oc2c1] |
| 186 | 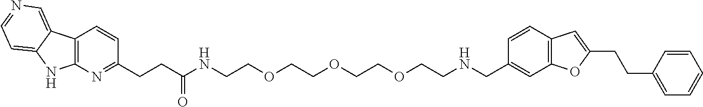 [SMILES: O=C(CCc1ccc2c(n1)[nH]c1ccncc12)NCCOCCOCCOCCNCc1ccc2cc(CCc3ccccc3)oc2c1] |
| 187 | 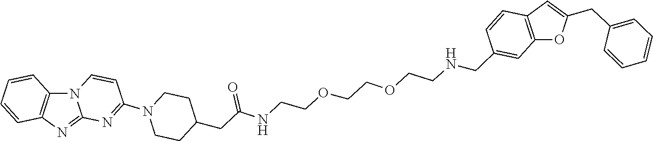 [SMILES: O=C(CC1CCN(c2ccn3c(n2)nc2ccccc23)CC1)NCCOCCOCCNCc1ccc2cc(Cc3ccccc3)oc2c1] |
| 188 | 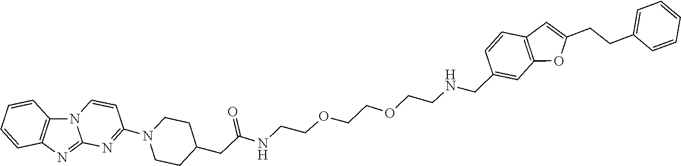 [SMILES: O=C(CC1CCN(c2ccn3c(n2)nc2ccccc23)CC1)NCCOCCOCCNCc1ccc2cc(CCc3ccccc3)oc2c1] |
| 189 | 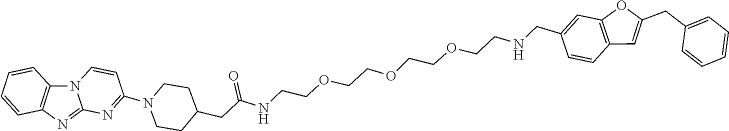 [SMILES: O=C(CC1CCN(c2ccn3c(n2)nc2ccccc23)CC1)NCCOCCOCCOCCNCc1ccc2cc(Cc3ccccc3)oc2c1] |
| 190 | 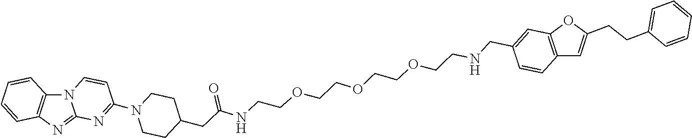 [SMILES: O=C(CC1CCN(c2ccn3c(n2)nc2ccccc23)CC1)NCCOCCOCCOCCNCc1ccc2cc(CCc3ccccc3)oc2c1] |
| 191 | 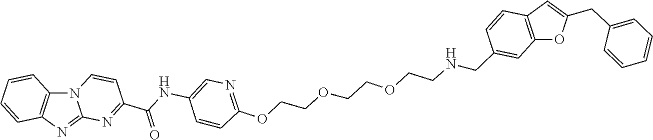 [SMILES: O=C(Nc1ccc(OCCOCCOCCNCc2ccc3cc(Cc4ccccc4)oc3c2)nc1)c1ccn2c(n1)nc1ccccc12] |
| 192 | 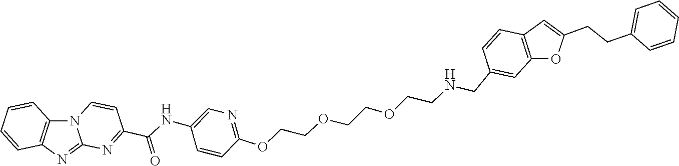 [SMILES: O=C(Nc1ccc(OCCOCCOCCNCc2ccc3cc(CCc4ccccc4)oc3c2)nc1)c1ccn2c(n1)nc1ccccc12] |
| 193 | 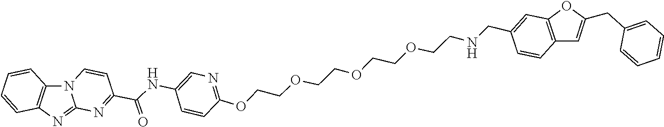 [SMILES: O=C(Nc1ccc(OCCOCCOCCOCCNCc2ccc3cc(Cc4ccccc4)oc3c2)nc1)c1ccn2c(n1)nc1ccccc12] |
| 194 | 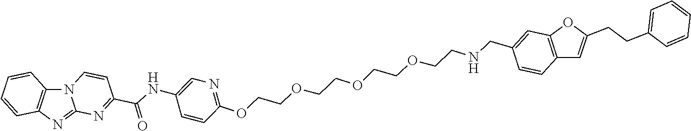 [SMILES: O=C(Nc1ccc(OCCOCCOCCOCCNCc2ccc3cc(CCc4ccccc4)oc3c2)nc1)c1ccn2c(n1)nc1ccccc12] |
| 195 | 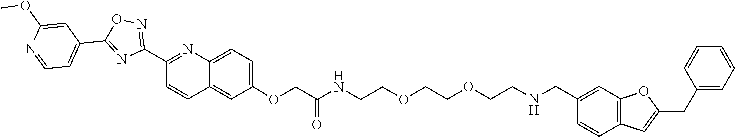 [SMILES: COc1cc(-c2nc(-c3ccc4cc(OCC(=O)NCCOCCOCCNCc5ccc6cc(Cc7ccccc7)oc6c5)ccc4n3)no2)ccn1] |
| 196 | 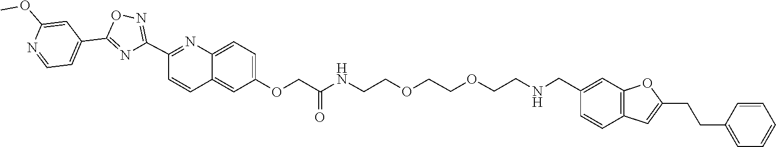 [SMILES: COc1cc(-c2nc(-c3ccc4cc(OCC(=O)NCCOCCOCCNCc5ccc6cc(CCc7ccccc7)oc6c5)ccc4n3)no2)ccn1] |
| 197 | 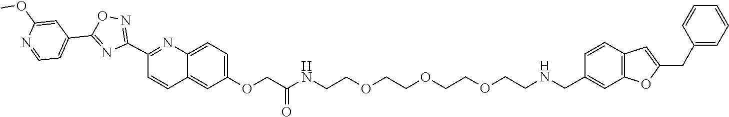 [SMILES: COc1cc(-c2nc(-c3ccc4cc(OCC(=O)NCCOCCOCCOCCNCc5ccc6cc(Cc7ccccc7)oc6c5)ccc4n3)no2)ccn1] |
| 198 | 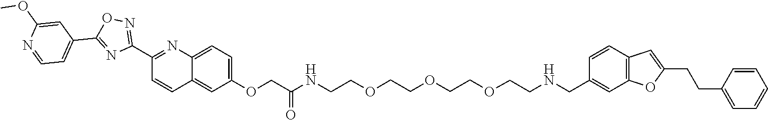 [SMILES: COc1cc(-c2nc(-c3ccc4cc(OCC(=O)NCCOCCOCCOCCNCc5ccc6cc(CCc7ccccc7)oc6c5)ccc4n3)no2)ccn1] |
| 199 | 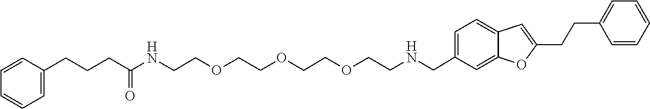 [SMILES: O=C(CCCc1ccccc1)NCCOCCOCCOCCNCc1ccc2cc(CCc3ccccc3)oc2c1] |
| 200 | 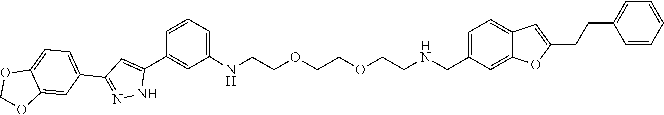 [SMILES: c1ccc(CCc2cc3ccc(CNCCOCCOCCNc4cccc(-c5cc(-c6ccc7c(c6)OCO7)n[nH]5)c4)cc3o2)cc1] |

### Compositions

The compounds of the present disclosure (e.g., a compound of formula (I), (I-A), (I-B), (I-C), (I-D), (I-A-1), (I-A-2), (I-A-3), (I-A-4), (I-B-1), (I-B-2), (I-B-3), (I-B-4), (I-C-1), (I-C-2), (I-C-3), (I-C-4), (I-D-1), (I-D-2), (I-D-3), (I-D-4), (I-A-1-a), (I-A-2-a), (I-A-3-a), (I-B-1-a), (I-B-2-a), (I-B-3-a), (I-C-1-a), (I-D-1-a), (II), (II-A), (II-B), (II-C), (II-D), (II-A-1), (II-A-2), (II-A-3), (II-A-4), (II-B-1), (II-B-2), (II-B-3), (II-B-4), (II-C-1), (II-C-2), (II-C-3), (II-C-4), (II-D-1), (II-D-2), (II-D-3), (II-D-4), (III), (III-A), (III-B), (III-C), (III-D), (III-A-1), (III-A-2), (III-A-3), (III-A-4), (III-B-1), (III-B-2), (III-B-3), (III-B-4), (III-C-1), (III-C-2), (III-C-3), (III-C-4), (III-D-1), (III-D-2), (III-D-3), or (III-D-4), or Table 1, 2, or 3, or a pharmaceutically acceptable salt, stereoisomer, or deuterated form thereof) may be used on their own but will generally be administered in the form of a pharmaceutical composition in which the compound (active ingredient) is in association with pharmaceutically acceptable adjuvant(s), diluents(s), or carrier(s). Conventional procedures for the selection and preparation of suitable pharmaceutical formulations are described in, for example, “Pharmaceuticals—The Science of Dosage Form Designs”, M. E. Aulton, Churchill Livingstone, 2nd Ed. 2002.

In some embodiments, the present disclosure provides a pharmaceutical composition comprising one or more compounds of the present disclosure (e.g., a compound of formula (I), (I-A), (I-B), (I-C), (I-D), (I-A-1), (I-A-2), (I-A-3), (I-A-4), (I-B-1), (I-B-2), (I-B-3), (I-B-4), (I-C-1) (I-C-2), (I-C-3), (I-C-4), (I-D-1), (I-D-2), (I-D-3), (I-D-4), (I-A-1-a), (I-A-2-a), (I-A-3-a), (I-B-1-a), (I-B-2-a), (I-B-3-a), (I-C-1-a), (I-D-1-a), (II), (II-A), (II-B), (II-C), (II-D), (II-A-1), (II-A-2), (II-A-3), (II-A-4), (II-B-1), (II-B-2), (II-B-3), (II-B-4), (II-C-1), (II-C-2), (II-C-3), (II-C-4), (II-D-1), (II-D-2), (II-D-3), (II-D-4), (III), (III-A), (III-B), (III-C), (III-D), (III-A-1), (III-A-2), (III-A-3), (III-A-4), (III-B-1), (III-B-2), (III-B-3), (III-B-4), (III-C-1), (III-C-2), (III-C-3), (III-C-4), (III-D-1), (III-D-2), (III-D-3), or (III-D-4), or Table 1, 2, or 3) or a pharmaceutically acceptable salt, stereoisomer, or deuterated form thereof.

Depending on the mode of administration, the pharmaceutical composition can comprise from 0.01 to 99% wt (percent by weight), from 0.05 to 95% wt, from 0.1 to 90% wt, from 0.2 to 80% wt, from 0.3 to 70% wt, from 0.4 to 60% wt, or from 0.5 to 50% wt, of active ingredient (e.g., a compound of formula (I), (I-A), (I-B), (I-C), (I-D), (I-A-1), (I-A-2), (I-A-3), (I-A-4), (I-B-1), (I-B-2), (I-B-3), (I-B-4), (I-C-1), (I-C-2), (I-C-3), (I-C-4), (I-D-1), (I-D-2), (I-D-3), (I-D-4), (I-A-1-a), (I-A-2-a), (I-A-3-a), (I-B-1-a), (I-B-2-a), (I-B-3-a), (I-C-1-a), (I-D-1-a), (II), (II-A), (II-B), (II-C), (II-D), (II-A-1), (II-A-2), (II-A-3), (II-A-4), (II-B-1), (II-B-2), (II-B-3), (II-B-4), (II-C-1), (II-C-2), (II-C-3), (II-C-4), (II-D-1), (II-D-2), (II-D-3), (II-D-4), (III), (III-A), (III-B), (III-C), (III-D), (III-A-1), (III-A-2), (III-A-3), (III-A-4), (III-B-1), (III-B-2), (III-B-3), (III-B-4), (III-C-1), (III-C-2), (III-C-3), (III-C-4), (III-D-1), (III-D-2), (III-D-3), or (III-D-4), or Table 1, 2, or 3, or a pharmaceutically acceptable salt, stereoisomer, or deuterated form thereof), all percentages by weight being based on total composition.

In embodiments, the present disclosure provides pharmaceutical composition(s) comprising one or more compounds of formula (I), (I-A), (I-B), (I-C), (I-D), (I-A-1), (I-A-2), (I-A-3), (I-A-4), (I-B-1), (I-B-2), (I-B-3), (I-B-4), (I-C-1), (I-C-2), (I-C-3), (I-C-4), (I-D-1), (I-D-2), (I-D-3), (I-D-4), (I-A-1-a), (I-A-2-a), (I-A-3-a), (I-B-1-a), (I-B-2-a), (I-B-3-a), (I-C-1-a), (I-D-1-a), (II), (II-A), (II-B), (II-C), (II-D), (II-A-1), (II-A-2), (II-A-3), (II-A-4), (II-B3-1), (II-B3-2), (II-B3-3), (II-B3-4), (II-C-1), (II-C-2), (II-C-3), (II-C-4), (II-D-1), (II-D-2), (II-D-3), (II-D-4), (III), (III-A), (III-B), (III-C), (III-D), (III-A-1), (III-A-2), (III-A-3), (III-A-4), (III-B3-1), (III-B-2), (III-B-3), (III-B-4), (III-C-1), (III-C-2), (III-C-3), (III-C-4), (III-D-1), (III-D-2), (III-D-3), or (III-D-4), or Table 1, 2, or 3, or a pharmaceutically acceptable salt, stereoisomer, or deuterated form thereof, and pharmaceutically acceptable adjuvant(s), diluent(s) or carrier(s).

In some embodiments, the present disclosure provides pharmaceutical composition(s) comprising one or more compounds of Table 1, 2, or 3, or a pharmaceutically acceptable salt, stereoisomer, or deuterated form thereof, and pharmaceutically acceptable adjuvant(s), diluent(s) or carrier(s).

The pharmaceutically acceptable excipients and adjuvants are added to the composition or formulation for a variety of purposes. In some embodiments, a pharmaceutical composition comprising one or more compounds disclosed herein, or a pharmaceutically acceptable salt, stereoisomer, or deuterated form thereof, further comprise a pharmaceutically acceptable carrier. In some embodiments, a pharmaceutically acceptable carrier includes a pharmaceutically acceptable excipient, binder, and/or diluent. In some embodiments, suitable pharmaceutically acceptable carriers include, but are not limited to, inert solid fillers or diluents and sterile aqueous or organic solutions. In some embodiments, suitable pharmaceutically acceptable excipients include, but are not limited to, water, salt solutions, alcohol, polyethylene glycols, gelatin, lactose, amylase, magnesium stearate, talc, silicic acid, viscous paraffin, and the like.

For the purposes of this disclosure, the compounds of the present disclosure can be formulated for administration by a variety of means including orally, parenterally, by inhalation spray, topically, transdermally, buccally, sublingually, or rectally in formulations containing pharmaceutically acceptable carriers, adjuvants, and vehicles. The term parenteral as used here includes subcutaneous, intravenous, intramuscular, and intraarterial injections with a variety of infusion techniques. Intraarterial and intravenous injection as used herein includes administration through catheters.

In some embodiments, the pharmaceutical composition can be formulated for oral administration. The oral formulations can be presented in discrete units, such as capsules, pills, cachets, lozenges, or tablets, each containing a predetermined amount of the active compound; as a powder or granules; as a solution or a suspension in an aqueous or non-aqueous liquid; or as an oil-in-water or water-in-oil emulsion.

In some embodiments, the pharmaceutical composition is formulated for parenteral administration (such as intravenous injection or infusion, subcutaneous or intramuscular injection). The parenteral formulations can be, for example, an aqueous solution, a suspension, or an emulsion.

In some embodiments, the pharmaceutical composition is formulated for inhalation. The inhalable formulations can be, for example, formulated as a nasal spray, dry powder, or an aerosol administrable through a metered-dose inhaler.

### Therapeutic Use

In embodiments, the compounds of the present disclosure are p62 modulators, and thus may be used in any disease area where p62 plays a role. In embodiments, the compounds of the present disclosure are NBR1 modulators, and thus may be used in any disease area where NBR1 plays a role. As such, in one aspect of the disclosure, a method of treatment is provided. The method of treatment, in one embodiment, comprises, administering to a subject in need thereof, a composition comprising an effective amount of a compound of formula (I), (I-A), (I-B), (I-C), (I-D), (I-A-1), (I-A-2), (I-A-3), (I-A-4), (I-B-1), (I-B-2), (I-B-3), (I-B-4), (I-C-1), (I-C-2), (I-C-3), (I-C-4), (I-D-1), (I-D-2), (I-D-3), (I-D-4), (I-A-1-a), (I-A-2-a), (I-A-3-a), (I-B-1-a), (I-B-2-a), (I-B-3-a), (I-C-1-a), (I-D-1-a), (II), (II-A), (II-B), (II-C), (II-D), (II-A-1), (II-A-2), (II-A-3), (II-A-4), (II-B3-1), (II-B3-2), (II-B3-3), (II-B3-4), (II-C-1), (II-C-2), (II-C-3), (II-C-4), (II-D-1), (II-D-2), (II-D-3), (II-D-4), (III), (III-A), (III-B), (III-C), (III-D), (III-A-1), (III-A-2), (III-A-3), (III-A-4), (III-B-1), (III-B-2), (III-B-3), (III-B-4), (III-C-1), (III-C-2), (III-C-3), (III-C-4), (III-D-1), (III-D-2), (III-D-3), or (III-D-4), or Table 1, 2, or 3, or a pharmaceutically acceptable salt, stereoisomer, or deuterated form thereof. In embodiments, the composition is administered to the patient for an administration period.

In embodiments, a compound or composition of the present disclosure is used in a method for modulating autophagy. In some embodiments, the present disclosure provides methods of modulating autophagy. In some embodiments, the method comprises contacting p62 (e.g., one or more amino acids present in p62 protein) with compounds or compositions of the present disclosure (e.g., compounds of formula (I), (I-A), (I-B), (I-C), (I-D), (I-A-1), (I-A-2), (I-A-3), (I-A-4), (I-B-1), (I-B-2), (I-B-3), (I-B-4), (I-C-1), (I-C-2), (I-C-3), (I-C-4), (I-D-1), (I-D-2), (I-D-3), (I-D-4), (I-A-1-a), (I-A-2-a), (I-A-3-a), (I-B-1-a), (I-B-2-a), (I-B-3-a), (I-C-1-a), (I-D-1-a), (II), (II-A), (II-B), (II-C), (II-D), (II-A-1), (II-A-2), (II-A-3), (II-A-4), (II-B3-1), (II-B3-2), (II-B3-3), (II-B3-4), (II-C-1), (II-C-2), (II-C-3), (II-C-4), (II-D-1), (II-D-2), (II-D-3), (II-D-4), (III), (III-A), (III-B), (III-C), (III-D), (III-A-1), (III-A-2), (III-A-3), (III-A-4), (III-B3-1), (III-B-2), (III-B-3), (III-B-4), (III-C-1), (III-C-2), (III-C-3), (III-C-4), (III-D-1), (III-D-2), (III-D-3), or (III-D-4), or Table 1, 2, or 3, or a pharmaceutically acceptable salt, stereoisomer, or deuterated form thereof). In some embodiments, the compound increases activity of p62, thereby causing autophagy. In some embodiments, the compound decreases activity of p62, thereby reducing autophagy.

In some embodiments, the method comprises contacting NBR1 (e.g., one or more amino acids present in NBR1 protein) with compounds or compositions of the present disclosure (e.g., compounds of formula (I), (I-A), (I-B), (I-C), (I-D), (I-A-1), (I-A-2), (I-A-3), (I-A-4), (I-B-1), (I-B-2), (I-B-3), (I-B-4), (I-C-1), (I-C-2), (I-C-3), (I-C-4), (I-D-1), (I-D-2), (I-D-3), (I-D-4), (I-A-1-a), (I-A-2-a), (I-A-3-a), (I-B-1-a), (I-B-2-a), (I-B-3-a), (I-C-1-a), (I-D-1-a), (II), (II-A), (II-B), (II-C), (II-D), (II-A-1), (II-A-2), (II-A-3), (II-A-4), (II-B3-1), (II-B3-2), (II-B-3), (II-B3-4), (II-C-1), (II-C-2), (II-C-3), (II-C-4), (II-D-1), (II-D-2), (II-D-3), (II-D-4), (III), (III-A), (III-B), (III-C), (III-D), (III-A-1), (III-A-2), (III-A-3), (III-A-4), (III-B-1), (III-B-2), (III-B-3), (III-B-4), (III-C-1), (III-C-2), (III-C-3), (III-C-4), (III-D-1), (III-D-2), (III-D-3), or (III-D-4), or Table 1, 2, or 3, or a pharmaceutically acceptable salt, stereoisomer, or deuterated form thereof). In some embodiments, the compound increases activity of NBR1, thereby causing autophagy. In some embodiments, the compound decreases activity of NBR1, thereby reducing autophagy.

In embodiments, a compound or composition of the present disclosure is administered to a subject in need thereof in a method for inducing autophagy. In some embodiments, the compound contacts p62, thereby inducing autophagy.

In embodiments, a compound or composition of the present disclosure is administered to a subject in need thereof in a method for inducing autophagy. In some embodiments, the compound contacts NBR1, thereby inducing autophagy.

In embodiments, a compound or composition of the present disclosure is administered to a subject in need thereof in a method for degrading target proteins, protein aggregates, protein complexes, lipids (e.g., lipid droplets), bacteria, or viruses. In some embodiments, a compound or composition of the present disclosure is administered to a subject in need thereof in a method for reducing the quantity of target proteins, protein aggregates, protein complexes, lipids, bacteria, or viruses.

In embodiments, a compound or composition of the present disclosure is administered to a subject in need thereof in a method for degrading a target protein. In some embodiments, a compound or composition of the present disclosure is administered to a subject in need thereof in a method for reducing the quantity of target proteins.

In embodiments, a compound or composition of the present disclosure is administered to a patient in a method for treating cancer (e.g., cancer metastasis), metabolic diseases, inflammation, neurodegenerative disorders, and infectious diseases.

In some embodiments, the cancer is a breast cancer, colorectal cancer, kidney cancer, ovarian cancer, gastric cancer, thyroid cancer, urothelial cancer, testicular cancer, cervical cancer, nasopharyngeal cancer, esophageal cancer, bile duct cancer, lung cancer, pancreatic cancer, prostate cancer, bone cancer, blood cancer, brain cancer, liver cancer, mesothelioma, melanoma, hematologic cancer, sarcoma, gastrointestinal stromal tumor, peripheral nerve sheath tumor, myeloma, mesothelioma, endometrial cancer, and/or leukemia.

In some embodiments, the cancer is breast cancer (e.g., ER negative breast cancer, triple negative breast cancer, basal-like breast cancers, HER2-positive breast cancers), kidney cancer (e.g., renal cell carcinoma (RCC)), prostate cancer, glioblastoma, or leukemia (e.g., chronic myelogenous leukemia, acute myelogenous leukemia, acute lymphoblastic leukemia).

In some embodiments, the neurodegenerative disorder is Alzheimer's disease, Parkinson's disease, and/or Huntington's Disease.

In some embodiments, the infectious disease is a bacterial infection and/or a virus infection.

### NUMBERED EMBODIMENTS OF THE DISCLOSURE

In addition to the disclosure above, the Examples below, and the appended claims, the disclosure sets forth the following numbered embodiments.

1. A compound of formula (I)

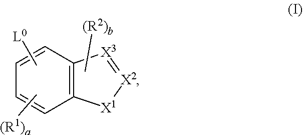

or a pharmaceutically acceptable salt, stereoisomer, or deuterated form thereof, wherein:
L⁰ is —C₁₋₆ alkylene-NR^{A}R^{B}, —C₁₋₆ alkylene-NR^{A}—C₁₋₆ alkylene-R^{B}, —C₁₋₆ alkylene-NR^{A}C(O)NR^{A}R^{B}, —C₁₋₆ alkylene-NR^{A}C(O)—R^{B}, —C₁₋₆ alkylene-O—C₁₋₆ alkylene-NR^{A}R^{B}, —C₃₋₈ cycloalkylene-NR^{A}R^{B}, —C(O)NR^{A}R^{B}, —O—C₁₋₆ alkylene-C(O)NR^{A}R^{B}, —O—C₁₋₆ alkylene-NR^{A}R^{B}, —NR^{A}C(O)R^{B}, —NR^{A}C(O)NR^{A}R^{B}, or —NR^{A}C(O)NR^{A}—C₁₋₆ alkylene-R^{B}, wherein the alkylene is optionally substituted with —OH or halogen;
each R¹ is independently C₁₋₆ alkyl, C₃-C₈ cycloalkyl, or halogen;
each R² is independently C₁₋₆ alkyl, C₃-C₈ cycloalkyl, or halogen;
X¹ is O or S;
one of X² or X³ is C-Q-R³ and the other is CH, C, or N as permitted by valency;
Q is absent, C₁₋₆ alkylene, —C₁₋₆ alkylene-C(O)—, C₃₋₈ cycloalkylene, —C₃₋₈ cycloalkylene-C(O)—, —C(O)—, or —S(O)₂—;
R³ is carbocycle, heterocycle, aryl, heteroaryl, or —NR^{X}R^{Y}, wherein the carbocycle, heterocycle, aryl, and heteroaryl are each optionally substituted with 1, 2, or 3 R⁴;
each R⁴ is independently halogen, —OH, C₁₋₆ alkyl, C₁₋₆ alkoxy, —C(O)—NR^{X}R^{Y}, or —C(O)O—(C₁₋₆ alkyl);
each R^{A} is independently H, C₁₋₆ alkyl, C₁₋₆ haloalkyl, C₃₋₈ cycloalkyl, C₃₋₈ hydroxycycloalkyl, aryl, or heterocycle;
each R^{B} is independently C₁₋₆ hydroxyalkyl, C₁₋₆ alkoxy, C₁₋₆ alkylene-NH₂, or C₁₋₆ alkylene-SH;
R^{X} is H, C₁₋₆ alkyl, C₁₋₆ haloalkyl, or C₁₋₆ hydroxyalkyl;
R^{Y} is C₁₋₆ alkyl, C₃₋₈ cycloalkyl, C₃₋₈ hydroxycycloalkyl, aryl, or heterocycle;
a is an integer of 0-3; and
b is an integer of 0 or 1.
   2. The compound of embodiment 1, having a structure of formula (I-A):

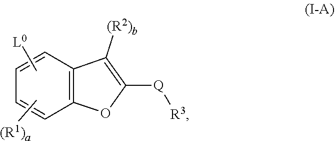

or a pharmaceutically acceptable salt, a stereoisomer, or a deuterated form thereof.
3. The compound of embodiment 1, having a structure of formula (I-B):

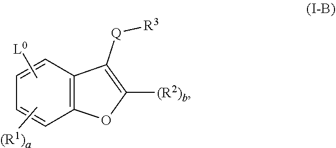

or a pharmaceutically acceptable salt, a stereoisomer, or a deuterated form thereof.
4. The compound of embodiment 1, having a structure of formula (I-C) or (I-D):

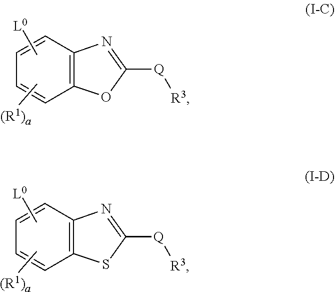

or a pharmaceutically acceptable salt, a stereoisomer, or a deuterated form thereof.
5. The compound of embodiment 1 or 2, having a structure of formula (I-A-1), (I-A-2), (I-A-3), or (I-A-4):

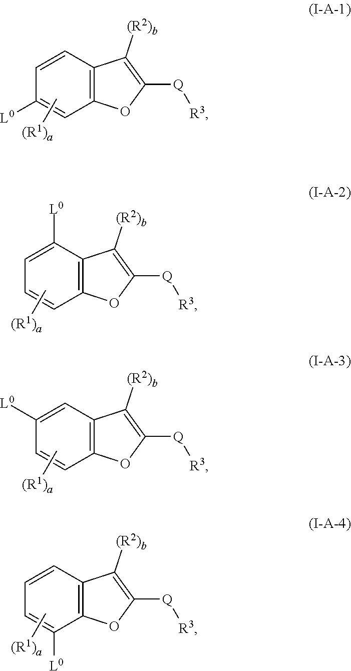

or a pharmaceutically acceptable salt, a stereoisomer, or a deuterated form thereof.
6. The compound of embodiment 1 or 3, having a structure of formula (I-B-1), (I-B-2), (I-B-3), or (I-B-4):

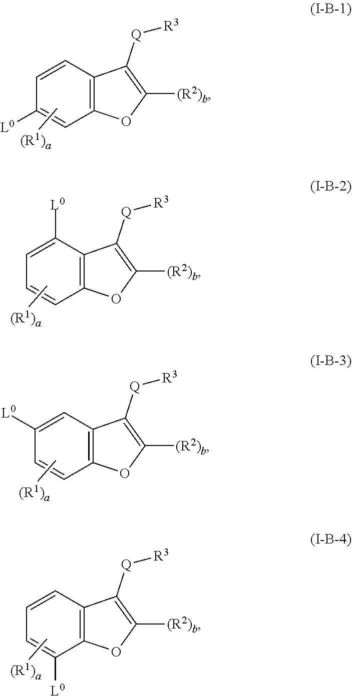

or a pharmaceutically acceptable salt, a stereoisomer, or a deuterated form thereof.
7. The compound of embodiment 1 or 4, having a structure of formula (I-C-1), (I-C-2), (I-C-3), (I-C-4), (I-D-1), (I-D-2), (I-D-3), or (I-D-4):

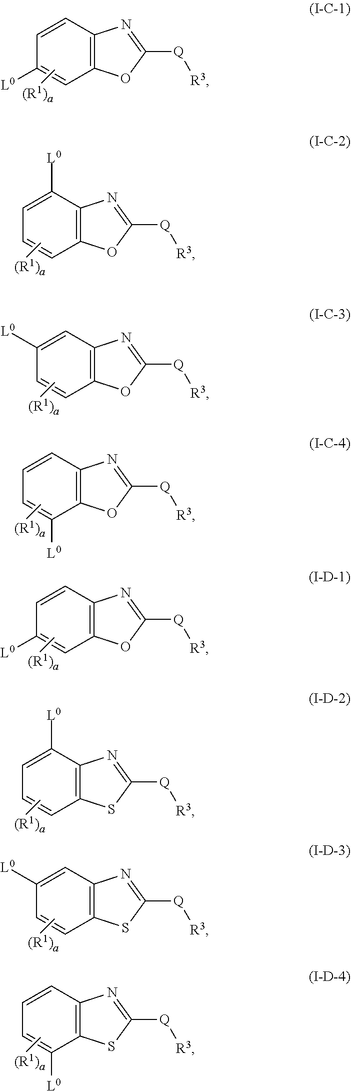

or a pharmaceutically acceptable salt, a stereoisomer, or a deuterated form thereof.
8. The compound of any one of embodiments 1-7, or a pharmaceutically acceptable salt, stereoisomer, or deuterated form thereof, wherein L⁰ is —(C₁₋₆ alkylene)-N(H)(C₁₋₆ hydroxyalkyl), —(C₁₋₆ alkylene)-N(C₁₋₆ alkyl)(C₁₋₆ hydroxyalkyl), —(C₁₋₆ alkylene)-N(H)(C₁₋₆ alkoxy), —(C₁₋₆ alkylene)-N(C₁₋₆ alkyl)(C₁₋₆ alkoxy), —(C₁₋₆ alkylene)-N(H)C(O)(C₁₋₆ alkoxy), —(C₁₋₆ alkylene)-N(C₁₋₆ alkyl)C(O)(C₁₋₆ alkoxy), —(C₁₋₆ alkylene)-N(H)C(O)N(H)(C₁₋₆ alkoxy), —(C₁₋₆ alkylene)-N(C₁₋₆ alkyl)C(O)N(C₁₋₆ alkyl)(C₁₋₆ alkoxy), —(C₃₋₈ cycloalkylene)N(H)(C₁₋₆ hydroxyalkyl), —(C₃₋₈ cycloalkylene)N(C₁₋₆ alkyl)(C₁₋₆ hydroxyalkyl), —O—(C₁₋₆ alkylene)-N(H)(C₁₋₆ hydroxyalkyl), —O—(C₁₋₆ alkylene)-N(C₁₋₆ alkyl)(C₁₋₆ hydroxyalkyl), —N(H)(C₁₋₆ hydroxyalkyl), —N(C₁₋₆ alkyl)(C₁₋₆ hydroxyalkyl), or —N(H)C(O)(C₁₋₆ hydroxyalkyl), —N(C₁₋₆ alkyl)C(O)(C₁₋₆ hydroxyalkyl),
wherein the alkylene or alkyl is optionally substituted with —OH or halogen.
   9. The compound of any one of claims 1-8, or a pharmaceutically acceptable salt, stereoisomer, or deuterated form thereof, wherein L⁰ is
—CH₂NHCH₂CH₂OH,
—CH₂NHCH₂CH(CH₃)OH,
—CH(CH₃)NHCH₂CH₂OH,

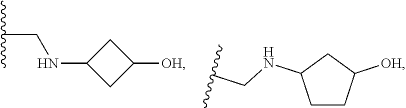 [SMILES: *CNC1CC(O)C1 ; *CNC1CCC(O)C1]

—CH₂NHCH₂CH₂OCH(CH₃)₂,
—CH₂OCH₂CH₂NHCH₃,
—CH₂OCH₂CH₂NHCH(CH₃)₂,
—C(O)NHCH₂CH₂OH,
—NHC(O)CH₂CH₂OH,
—NHC(O)CH₂OH,
—CH₂NHC(O)NHCH₂CH₂OH,

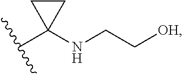 [SMILES: *C1(NCCO)CC1]

—NHC(O)NHCH₂CH₂OH,
—OCH₂CH(OH)CH₂NHCH(CH₃)₂,
—OCH₂CH₂C(O)NHCH₃,
—CH₂NHC(O)CH₂OH,
—CH₂NHC(O)CH₂CH₂OH, or
—NHC(O)NHCH₂CH₂OCH₃.
   10. The compound of any one of embodiments 1-9, or a pharmaceutically acceptable salt, stereoisomer, or deuterated form thereof, wherein L⁰ is —CH₂NHCH₂CH₂OH, —CH₂NHCH₂CH(CH₃)OH, —CH₂OCH₂CH₂NHCH₃, —OCH₂CH(OH)CH₂NHCH(CH₃)₂, —CH₂OCH₂CH₂NHCH(CH₃)₂, or

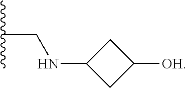 [SMILES: *CNC1CC(O)C1]

11. The compound of any one of embodiments 1, 2, 5, and 10, having a structure of formula (I-A-1-a), (I-A-2-a), or (I-A-3-a):

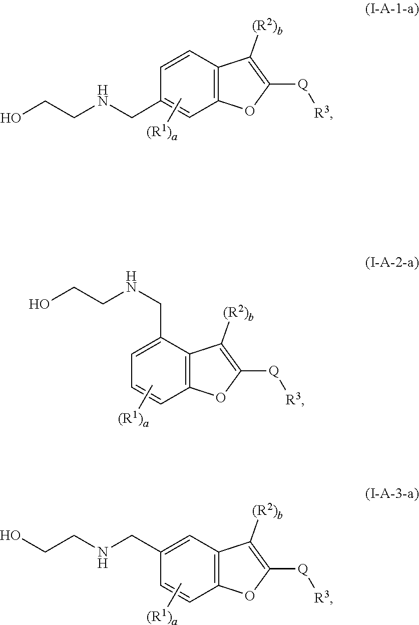

or a pharmaceutically acceptable salt, a stereoisomer, or a deuterated form thereof.
12. The compound of any one of embodiments 1, 3, 6, and 10, having a structure of formula (I-B-1-a), (I-B-2-a), or (I-B-3-a):

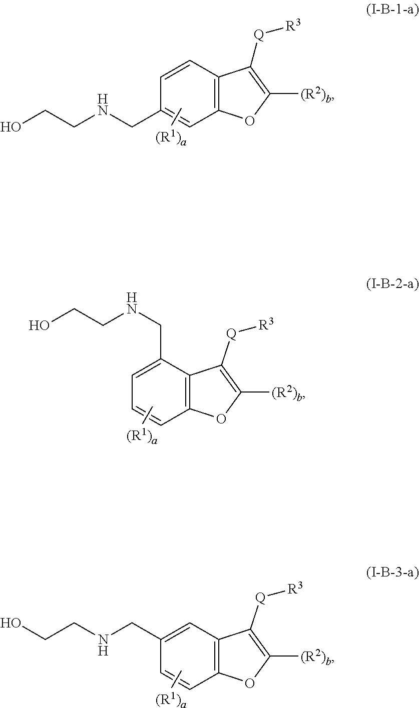

or a pharmaceutically acceptable salt, a stereoisomer, or a deuterated form thereof.
13. The compound of any one of embodiments 1, 4, 7, and 10, having a structure of formula (I-C-1-a) or (I-D-1-a):

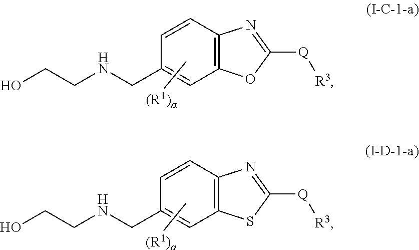

or a pharmaceutically acceptable salt, a stereoisomer, or a deuterated form thereof.
14. The compound of any one of the preceding embodiments, wherein at least one H in L⁰ is replaced by conjugate comprising a ligand that binds to a protein, a protein aggregate, a protein complex, or a lipid.
15. A compound of formula (II)

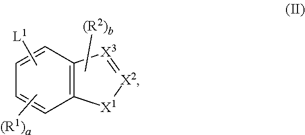

or a pharmaceutically acceptable salt, stereoisomer, or deuterated form thereof,
wherein:
L¹ is C₂-C₅₀ alkylene-R⁵, C₂-C₂₅ alkenylene-R⁵, C₂-C₂₅ alkynylene-R⁵, wherein 1-25 methylene groups of L¹ are optionally and independently replaced by —N(H)—, —N(C₁-C₆ alkyl)-, —N(C₃-C₈ cycloalkyl)-, —O—, —C(O)—, —C(O)O—, —S—, —S(O)—, —S(O)₂—, —S(O)₂N(C₁-C₆ alkyl)-, —S(O)₂N(C₃-C₈ cycloalkyl)-, —N(H)C(O)—, —N(C₁-C₆ alkyl)C(O)—, —N(C₃-C₈ cycloalkyl)C(O)—, —N(H)C(O)N(H)—, —N(C₁-C₆ alkyl)C(O)N(H)—, —N(H)C(O)N(C₁-C₆ alkyl)-, —N(C₁-C₆ alkyl)C(O)N(C₁-C₆ alkyl)-, —C(O)N(H)—, —C(O)N(C₁-C₆ alkyl)-, —C(O)N(C₃-C₈ cycloalkyl)-, arylene, heteroarylene, heterocyclylene, C₃-C₈ cycloalkylene, or C₃-C₈ cycloalkenylene, wherein the alkylene, alkenylene, alkynylene, alkyl, arylene, heteroarylene, and heterocyclylene are each optionally and independently substituted with 1, 2, or 3 R^{Z}, wherein each R^{Z} is independently —OH, C₁₋₆ alkyl, or halogen;
each R¹ is independently C₁₋₆ alkyl, C₃-C₈ cycloalkyl, or halogen;
each R² is independently C₁₋₆ alkyl, C₃-C₈ cycloalkyl, or halogen;
X¹ is O or S;
one of X² or X³ is C-Q-R³ and the other is CH, C, or N as permitted by valency;
Q is absent, C₁₋₆ alkylene, —C₁₋₆ alkylene-C(O)—, C₃₋₈ cycloalkylene, —C₃₋₈ cycloalkylene-C(O)—, —C(O)—, or —S(O)₂—;
R³ is carbocycle, heterocycle, aryl, heteroaryl, or —NR^{X}R^{Y}, wherein the carbocycle, heterocycle, aryl, and heteroaryl are each optionally substituted with 1, 2, or 3 R⁴;
each R⁴ is independently halogen, —OH, C₁₋₆ alkyl, C₁₋₆ alkoxy, —C(O)—NR^{X}R^{Y}, or —C(O)O—(C₁₋₆ alkyl);
each R⁵ is independently a bond, a leaving group, a protecting group, H, alkynyl, aryl, or heteroaryl;
R^{X} is H, C₁₋₆ alkyl, C₁₋₆ haloalkyl, or C₁₋₆ hydroxyalkyl;
R^{Y} is C₁₋₆ alkyl, C₃₋₈ cycloalkyl, C₃₋₈ hydroxycycloalkyl, aryl, or heterocycle;
a is an integer of 0-3; and
b is an integer of 0 or 1.
   16. The compound of embodiment 15, or a pharmaceutically acceptable salt, a stereoisomer, or a deuterated form thereof, wherein the compound has a structure of formula (II-A):

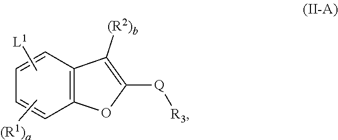

or a pharmaceutically acceptable salt, a stereoisomer, or a deuterated form thereof.
17. The compound of embodiment 15, or a pharmaceutically acceptable salt, a stereoisomer, or a deuterated form thereof, wherein the compound has a structure of formula (II-B):

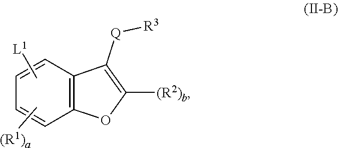

or a pharmaceutically acceptable salt, a stereoisomer, or a deuterated form thereof.
18. The compound of embodiment 15, or a pharmaceutically acceptable salt, a stereoisomer, or a deuterated form thereof, wherein the compound has a structure of formula (II-C) or (II-D):

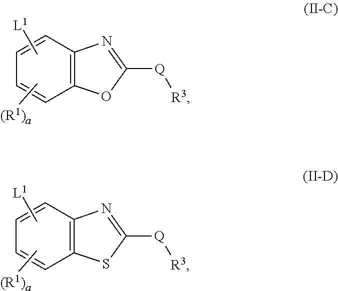

or a pharmaceutically acceptable salt, a stereoisomer, or a deuterated form thereof.
19. The compound of embodiment 16, or a pharmaceutically acceptable salt, a stereoisomer, or a deuterated form thereof, wherein the compound has a structure of formula (II-A-1), (II-A-2), (II-A-3), or (II-A-4):

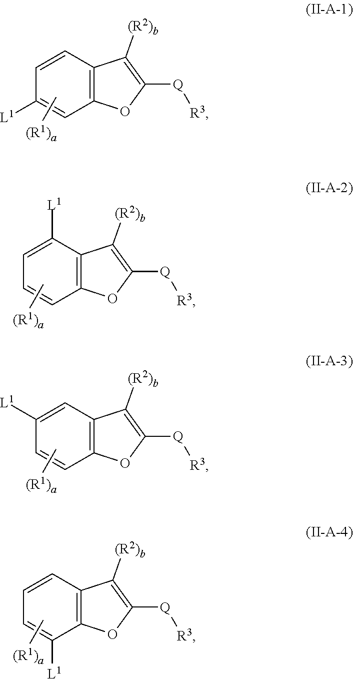

or a pharmaceutically acceptable salt, a stereoisomer, or a deuterated form thereof.
20. The compound of embodiment 17, or a pharmaceutically acceptable salt, a stereoisomer, or a deuterated form thereof, wherein the compound has a structure of formula (II-B-1), (II-B-2), (II-B-3), or (II-B-4):

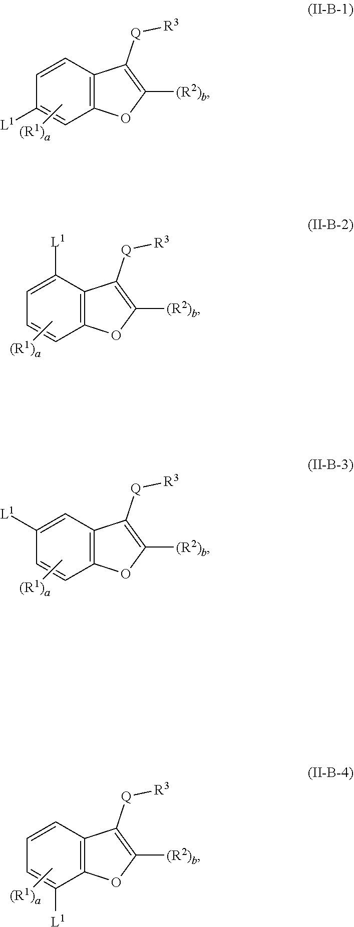

or a pharmaceutically acceptable salt, a stereoisomer, or a deuterated form thereof.
21. The compound of embodiment 18, or a pharmaceutically acceptable salt, a stereoisomer, or a deuterated form thereof, wherein the compound has a structure of formula (II-C-1), (II-C-2), (II-C-3), (II-C-4), (II-D-1), (II-D-2), (II-D-3), or (II-D-4):

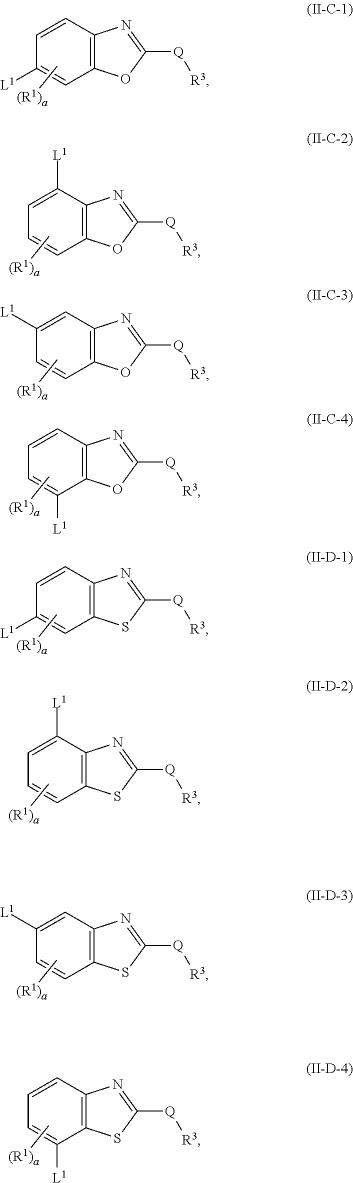

or a pharmaceutically acceptable salt, a stereoisomer, or a deuterated form thereof.
22. The compound of any one of embodiments 15-21, or a pharmaceutically acceptable salt, a stereoisomer, or a deuterated form thereof, wherein 1-25 methylene groups of L¹ are optionally and independently replaced by —N(H)—, —N(C₁-C₆ alkyl)-, —N(C₃-C₈ cycloalkyl)-, —O—, —C(O)—, —C(O)O—, —N(H)C(O)—, —N(C₁-C₆ alkyl)C(O)—, —N(C₃-C₈ cycloalkyl)C(O)—, —C(O)N(H)—, —C(O)N(C₁-C₆ alkyl)-, —C(O)N(C₃-C₈ cycloalkyl)-, arylene, heteroarylene, heterocyclylene, or C₃-C₈ cycloalkylene, wherein each arylene or heteroarylene is optionally and independently substituted with 1 or 2 R^{Z}.
23. The compound of any one of embodiments 15-22, or a pharmaceutically acceptable salt, a stereoisomer, or a deuterated form thereof, wherein L¹ is:
—(C₁₋₆ alkylene)-NH(CH₂CH₂O)ₘ—(CH₂)ₙ—C≡CH,
—O—(C₁₋₆ alkylene)-CH(OH)—(C₁₋₆ alkylene)-NH(CH₂CH₂O)ₘ—(CH₂)ₙ—C≡CH,
—(C₁₋₆ alkylene)-NH(CH₂CH₂O)ₘ—(CH₂)ₙ—NH—C(O)O—(C₁₋₆ alkyl),
—(C₁₋₆ alkylene)-NH(CH₂CH₂O)ₘ—(CH₂)ₙ—N(C₁₋₆ alkyl)-C(O)O—(C₁₋₆ alkyl),
—(C₁₋₆ alkylene)-NH(CH₂CH₂O)ₘ—(CH₂)ₙ-HET^{A}
—(C₁₋₆ alkylene)-NH(CH₂CH₂O)ₘ—(CH₂)ₙ—NH—(C₁₋₆ alkyl),
—(C₁₋₆ alkylene)-NH(CH₂CH₂O)ₘ—(CH₂)ₙ—C(O)O—(C₁₋₆ alkyl),
—O—(C₁₋₆ alkylene)-CH(OH)—(C₁₋₆ alkylene)-NH(CH₂CH₂O)ₘ—(CH₂)ₙ—C(O)O—(C₁₋₆ alkyl), or
—(C₁₋₆ alkylene)-NH(CH₂CH₂O)ₘ—(CH₂)ₙ—O—C₆₋₁₀ aryl;
wherein HET^{A} is a 4-10 membered heterocycle containing 1-3 N, and is optionally substituted with —C(O)—(C₁₋₆ alkyl), —C(O)O—(C₁₋₆ alkyl), —(C₁₋₆ alkyl)-O—C₆₋₁₀ aryl, or C₁₋₆ alkyl;
m is an integer of 1-12; and
n is an integer of 0-6.
   24. The compound of embodiment 23, or a pharmaceutically acceptable salt, a stereoisomer, or a deuterated form thereof, wherein HET^{A} is piperidinyl or piperazinyl optionally substituted with —C(O)—(C₁₋₆ alkyl), —C(O)O—(C₁₋₆ alkyl), or —(C₁₋₆ alkyl)-O—C₆₋₁₀ aryl.
   25. The compound of embodiment 23 or 24, or a pharmaceutically acceptable salt, a stereoisomer, or a deuterated form thereof, wherein m is an integer of 1-6.
   26. The compound of embodiment 25, or a pharmaceutically acceptable salt, a stereoisomer, or a deuterated form thereof, wherein m is an integer of 1-4.
   27. The compound of any one of embodiments 23-26, or a pharmaceutically acceptable salt, a stereoisomer, or a deuterated form thereof, wherein n is an integer of 0-3.
   28. The compound of embodiment 23, or a pharmaceutically acceptable salt, a stereoisomer, or a deuterated form thereof, wherein L¹ is
—CH₂NH—(CH₂CH₂O)₂—(CH₂)₂—C≡CH,
—CH₂NH—(CH₂CH₂O)₃—(CH₂)₂—C≡CH,
—O—CH₂—CH(OH)—CH₂—NH—(CH₂CH₂O)₂—(CH₂)₂—C≡CH,
—CH₂NH—(CH₂CH₂O)₃—(CH₂)₂—NHC(O)OC(CH₃)₃,
—CH₂NH—(CH₂CH₂O)₃—(CH₂)₃—NCH₃C(O)OC(CH₃)₃,
—CH₂NH—(CH₂CH₂O)₄—(CH₂)₃—NCH₃C(O)OC(CH₃)₃,

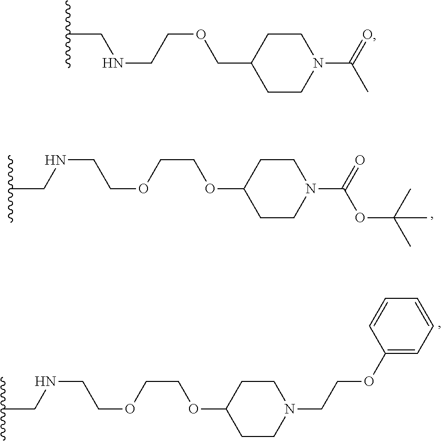 [SMILES: *CNCCOCC1CCN(C(C)=O)CC1 ; *CNCCOCCOC1CCN(C(=O)OC(C)(C)C)CC1 ; *CNCCOCCOC1CCN(CCOc2ccccc2)CC1]

—CH₂NH—(CH₂CH₂O)₂—(CH₂)₂—NHCH₃,
—CH₂NH—(CH₂CH₂O)₃—(CH₂)₂—NHCH₃,
—CH₂NH—(CH₂CH₂O)₂—(CH₂)₂—C(O)OCH₃,
—O—CH₂—CH(OH)—CH₂NH—(CH₂CH₂O)—(CH₂)₂—C(O)OCH₃,

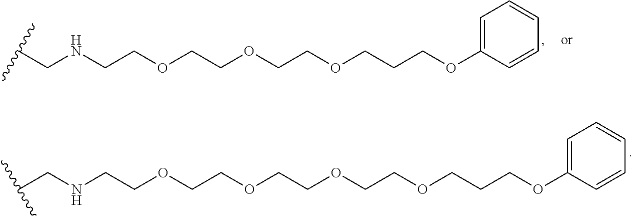 [SMILES: *CNCCOCCOCCOCCCOc1ccccc1 ; *CNCCOCCOCCOCCOCCCOc1ccccc1]

29. The compound of any one of embodiments 15-22, or a pharmaceutically acceptable salt, a stereoisomer, or a deuterated form thereof, wherein L¹ comprises a bivalent moiety selected from:

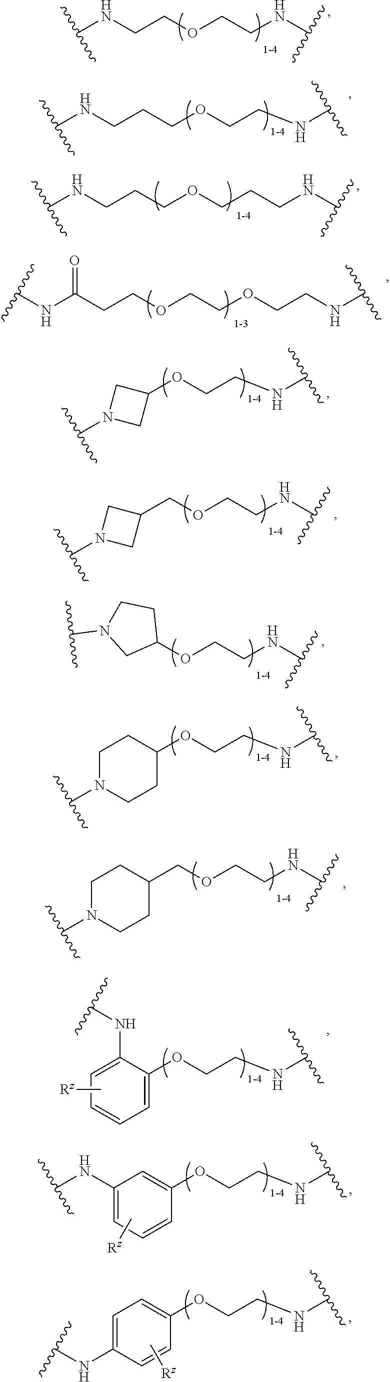

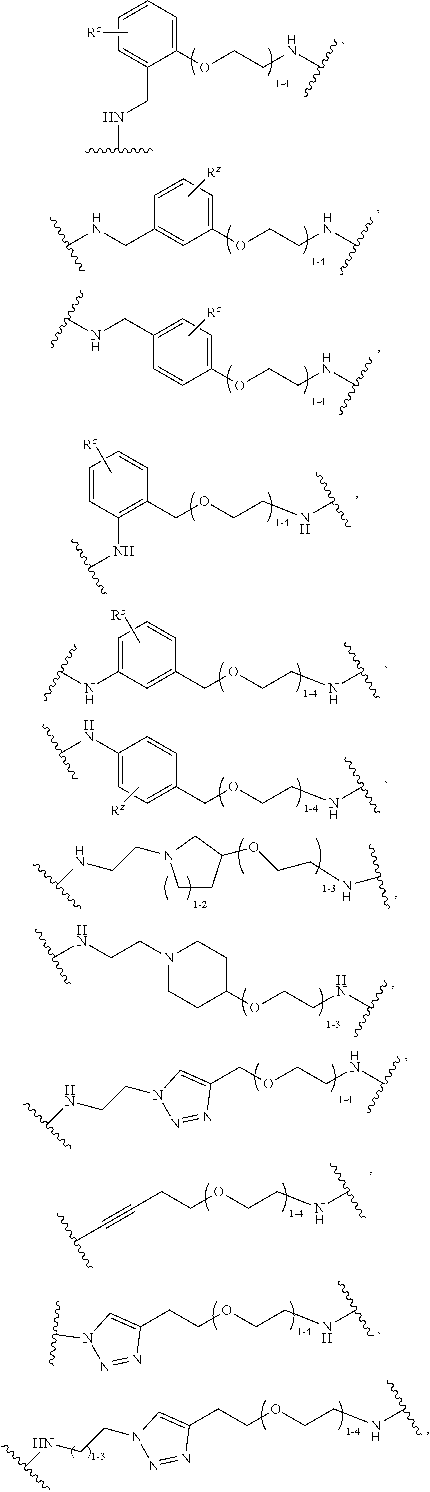

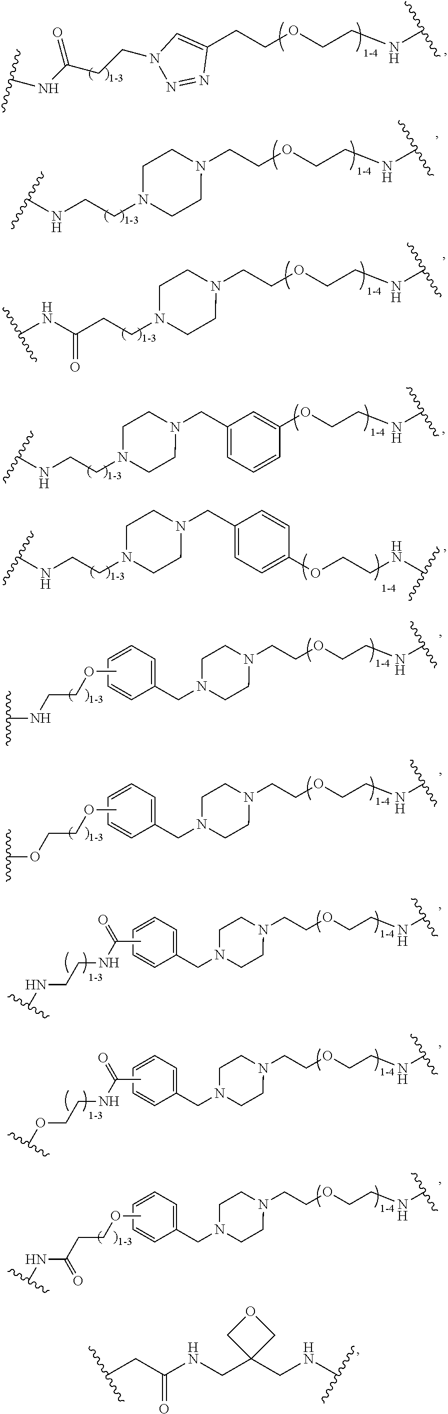

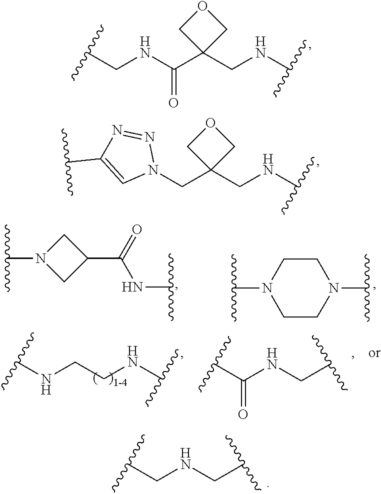

30. A compound of formula (III)

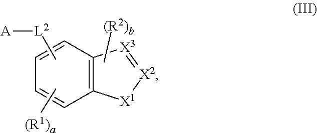

or a pharmaceutically acceptable salt, stereoisomer, or deuterated form thereof,
wherein:
A is a ligand that binds to a protein, a protein aggregate, a protein complex, or a lipid;
L² is C₂-C₅₀ alkylene, C₂-C₂₅ alkenylene, C₂-C₂₅ alkynylene, or —(C₂-C₅₀ alkylene)-arylene, wherein 1-25 methylene groups of L² are optionally and independently replaced by —N(H)—, —N(C₁-C₆ alkyl)-, —N(C₃-C₈ cycloalkyl)-, —O—, —C(O)—, —C(O)O—, —S—, —S(O)—, —S(O)₂—, —S(O)₂N(C₁-C₆ alkyl)-, —S(O)₂N(C₃-C₈ cycloalkyl)-, —N(H)C(O)—, —N(C₁-C₆ alkyl)C(O)—, —N(C₃-C₈ cycloalkyl)C(O)—, —N(H)C(O)N(H)—, —N(C₁-C₆ alkyl)C(O)N(H)—, —N(H)C(O)N(C₁-C₆ alkyl)-, —N(C₁-C₆ alkyl)C(O)N(C₁-C₆ alkyl)-, —C(O)N(H)—, —C(O)N(C₁-C₆ alkyl)-, —C(O)N(C₃-C₈ cycloalkyl)-, arylene, heteroarylene, heterocyclylene, C₃-C₈ cycloalkylene, or C₃-C₈ cycloalkenylene, wherein the alkylene, alkenylene, alkynylene, alkyl, arylene, heteroarylene, and heterocyclylene are each optionally and independently substituted with 1, 2, or 3 R^{Z}, wherein each R^{Z} is independently OH, C₁₋₆ alkyl, or halogen;
each R¹ is independently C₁₋₆ alkyl, C₃-C₈ cycloalkyl, or halogen;
each R² is independently C₁₋₆ alkyl, C₃-C₈ cycloalkyl, or halogen;
X¹ is O or S;
one of X² or X³ is C-Q-R³ and the other is CH, C, or N as permitted by valency;
Q is absent, C₁₋₆ alkylene, —C₁₋₆ alkylene-C(O)—, C₃₋₈ cycloalkylene, —C₃₋₈ cycloalkylene-C(O)—, —C(O)—, or —S(O)₂—;
R³ is carbocycle, heterocycle, aryl, heteroaryl, or —NR^{X}R^{Y}, wherein the carbocycle, heterocycle, aryl, and heteroaryl are each optionally substituted with 1, 2, or 3 R⁴;
each R⁴ is independently halogen, —OH, C₁₋₆ alkyl, C₁₋₆ alkoxy, —C(O)—NR^{X}R^{Y}, or —C(O)O—(C₁₋₆ alkyl);
R^{X} is H, C₁₋₆ alkyl, C₁₋₆ haloalkyl, or C₁₋₆ hydroxyalkyl;
R^{Y} is C₁₋₆ alkyl, C₃₋₈ cycloalkyl, C₃₋₈ hydroxycycloalkyl, aryl, or heterocycle;
a is an integer of 0-3; and
b is an integer of 0 or 1.
   31. The compound of embodiment 30, or a pharmaceutically acceptable salt, a stereoisomer, or a deuterated form thereof, wherein the compound has a structure of formula (III-A):

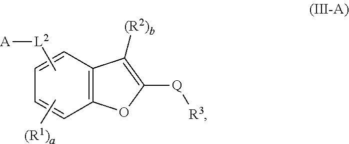

or a pharmaceutically acceptable salt, a stereoisomer, or a deuterated form thereof.
32. The compound of embodiment 30, or a pharmaceutically acceptable salt, a stereoisomer, or a deuterated form thereof, wherein the compound has a structure of formula (III-B):

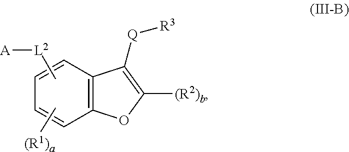

or a pharmaceutically acceptable salt, a stereoisomer, or a deuterated form thereof.
33. The compound of embodiment 30, or a pharmaceutically acceptable salt, a stereoisomer, or a deuterated form thereof, wherein the compound has a structure of formula (III-C) or (III-D3):

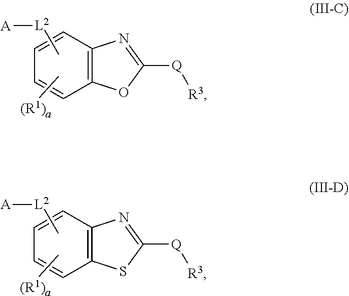

or a pharmaceutically acceptable salt, a stereoisomer, or a deuterated form thereof.
34. The compound of embodiment 31, or a pharmaceutically acceptable salt, a stereoisomer, or a deuterated form thereof, wherein the compound has a structure of formula (III-A-1), (III-A-2), (III-A-3), or (III-A-4):

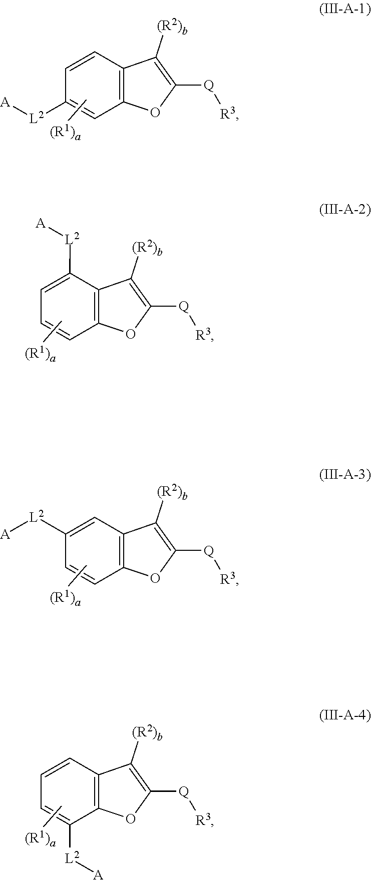

or a pharmaceutically acceptable salt, a stereoisomer, or a deuterated form thereof.
35. The compound of embodiment 32, or a pharmaceutically acceptable salt, a stereoisomer, or a deuterated form thereof, wherein the compound has a structure of formula (III-B-1), (III-B-2), (III-B-3), or (III-B-4):

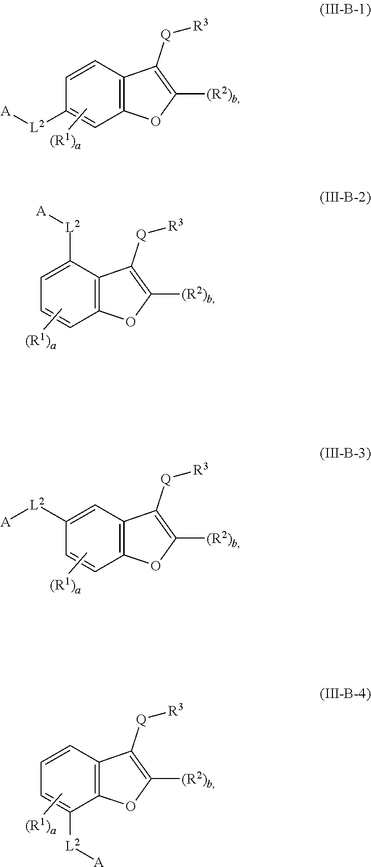

or a pharmaceutically acceptable salt, a stereoisomer, or a deuterated form thereof.
36. The compound of embodiment 33, or a pharmaceutically acceptable salt, a stereoisomer, or a deuterated form thereof, wherein the compound has a structure of formula (III-C-1), (III-C-2), (III-C-3), (III-C-4), (III-D-1), (III-D-2), (III-D-3), or (III-D-4):

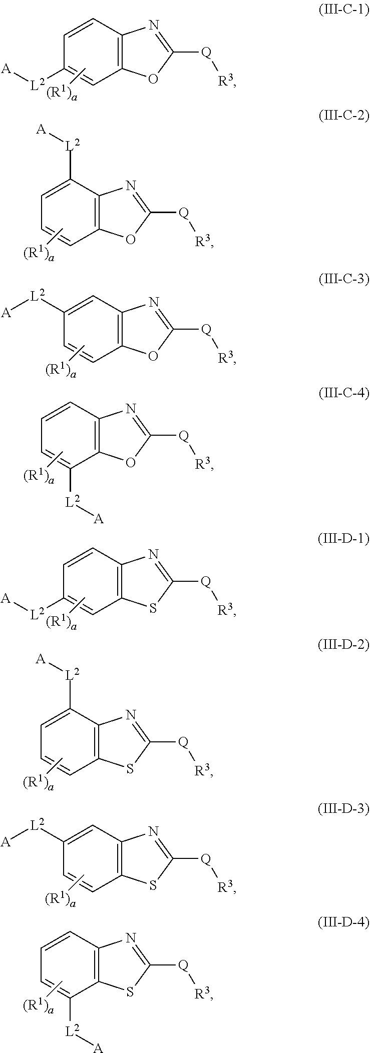

or a pharmaceutically acceptable salt, a stereoisomer, or a deuterated form thereof.
37. The compound of any one of embodiments 31-36, or a pharmaceutically acceptable salt, a stereoisomer, or a deuterated form thereof, wherein 1-25 methylene groups of L² are optionally and independently replaced by —N(H)—, —N(C₁-C₆ alkyl)-, —N(C₃-C₈ cycloalkyl)-, —O—, —C(O)—, —C(O)O—, —N(H)C(O)—, —N(C₁-C₆ alkyl)C(O)—, —N(C₃-C₈ cycloalkyl)C(O)—, —C(O)N(H)—, —C(O)N(C₁-C₆ alkyl)-, —C(O)N(C₃-C₈ cycloalkyl)-, arylene, heteroarylene, heterocyclylene, or C₃-C₈ cycloalkylene, wherein each arylene or heteroarylene is optionally and independently substituted with 1 or 2 R^{Z}.
38. The compound of any one of embodiments 30-37, or a pharmaceutically acceptable salt, a stereoisomer, or a deuterated form thereof, wherein L² is:
—(C₁₋₆ alkylene)-NH(CH₂CH₂O)ₘ—(CH₂)ₙ—NH—,
—(C₁₋₆ alkylene)-NH(CH₂CH₂O)ₘ—(CH₂)ₙ—N(C₁₋₆ alkyl)-,
—(C₁₋₆ alkylene)-NH(CH₂CH₂O)ₘ—(CH₂)ₙ—,
—(C₁₋₆ alkylene)-NH(CH₂CH₂O)ₘ—(CH₂)ₙ—NH—C(O)—(C₁₋₆ alkylene)-O—, or
—(C₁₋₆ alkylene)-NH(CH₂CH₂O)ₘ—(CH₂)—HET^{B}_
wherein HET^{B} is a 4-10 membered heterocyclylene containing 1-3 N, and is optionally substituted with —C(O)—(C₁₋₆ alkyl), —C(O)O—(C₁₋₆ alkyl), —(C₁₋₆ alkyl)-O—C₆₋₁₀ aryl, or C₁₋₆ alkyl;
m is an integer of 1-12; and
n is an integer of 0-6.
   39. The compound of any one of embodiments 30-38, or a pharmaceutically acceptable salt, a stereoisomer, or a deuterated form thereof, wherein L² is:
—(CH₂)—NH(CH₂CH₂O)—(CH₂)₂—NH—,
—(CH₂)—NH(CH₂CH₂O)₂—(CH₂)₂—NH—,
—(CH₂)—NH(CH₂CH₂O)₃—(CH₂)₂—NH—,
—(CH₂)—NH(CH₂CH₂O)₃—(CH₂)₃—NCH₃—,
—(CH₂)—NH(CH₂CH₂O)₄—(CH₂)₃—NCH₃—,
—(CH₂)—NH(CH₂CH₂O)₃—,
—(CH₂)—NH(CH₂CH₂O)₄—,
—(CH₂)—NH(CH₂CH₂O)₃—(CH₂)₂—NH—C(O)CH₂—O—,
—(CH₂)—NH(CH₂CH₂O)₃—(CH₂)₂—,
—(CH₂)—NH(CH₂CH₂O)₃—(CH₂)₃—,

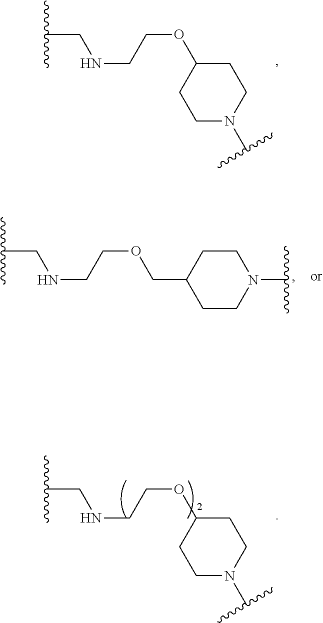

40. The compound of any one of embodiments 30-37, or a pharmaceutically acceptable salt, a stereoisomer, or a deuterated form thereof, wherein L² is:

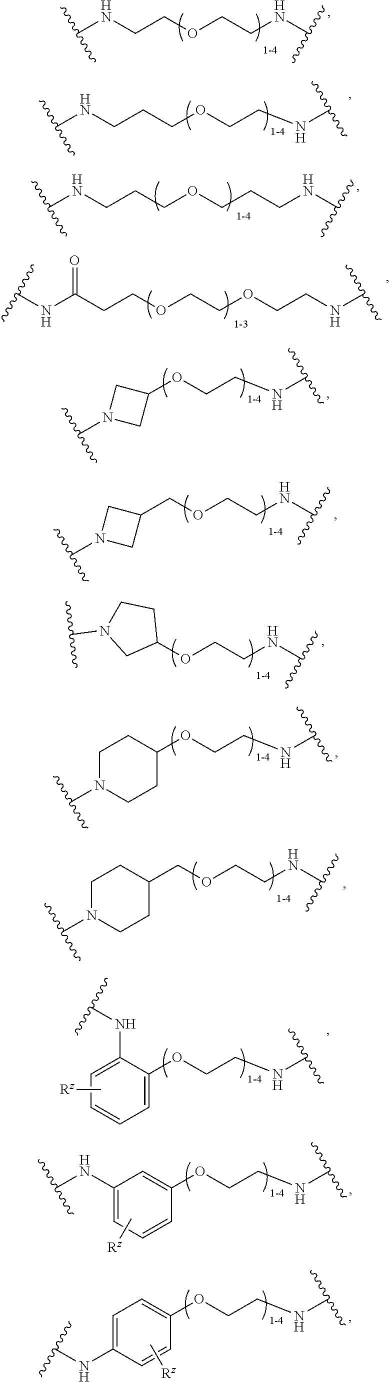

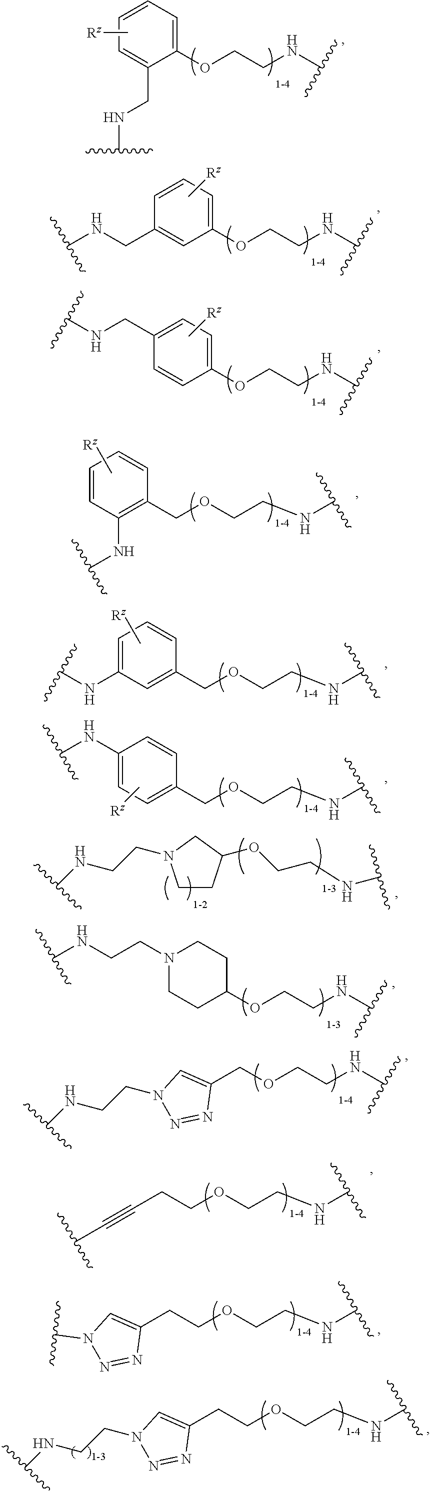

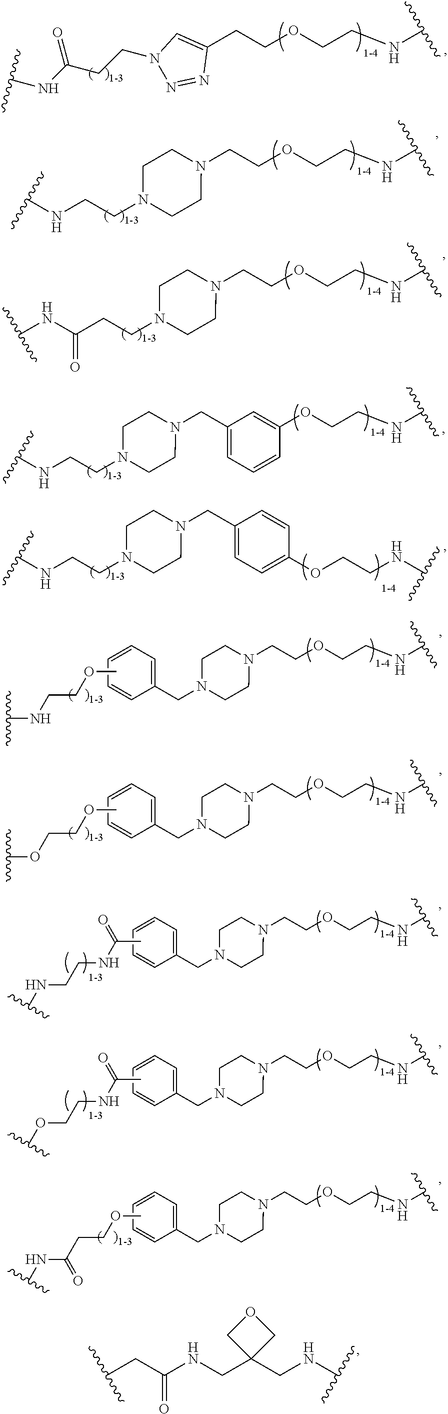

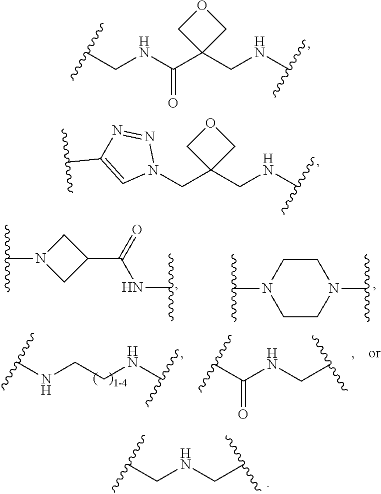

41. The compound of any one of embodiment 30-40, or a pharmaceutically acceptable salt, a stereoisomer, or a deuterated form thereof, wherein the protein is a protein that is associated with cancer.
42. The compound of embodiment 41, or a pharmaceutically acceptable salt, a stereoisomer, or a deuterated form thereof, wherein the protein associated with cancer comprises a mutation or a fusion.
43. The compound of embodiment 41 or 42, or a pharmaceutically acceptable salt, a stereoisomer, or a deuterated form thereof, wherein the protein associated with cancer is BRD4.
44. The compound of any one of embodiments 30-40, or a pharmaceutically acceptable salt, a stereoisomer, or a deuterated form thereof, wherein the protein is a protein associated with a metabolic disease.
45. The compound of any one of embodiments 30-40, or a pharmaceutically acceptable salt, a stereoisomer, or a deuterated form thereof, wherein the protein is a protein associated with inflammation.
46. The compound of any one of embodiments 30-40, or a pharmaceutically acceptable salt, a stereoisomer, or a deuterated form thereof, wherein the protein is present in bacteria.
47. The compound of any one of embodiments 30-40, or a pharmaceutically acceptable salt, a stereoisomer, or a deuterated form thereof, wherein the protein is present in a virus particle.
48. The compound of any one of embodiments 30-40, or a pharmaceutically acceptable salt, a stereoisomer, or a deuterated form thereof, wherein the protein aggregate is an alpha-synuclein protein aggregate, a mutant Huntingtin protein aggregate, an amyloid protein aggregate, or a TDP-43 protein aggregate.
49. The compound of any one of embodiments 30-40, or a pharmaceutically acceptable salt, a stereoisomer, or a deuterated form thereof, wherein the protein is an intracellular protein.
50. The compound of any one of embodiments 30-40, wherein A is aryl, arylene-heteroaryl, arylene, heteroarylene, aryl, heteroarylene-aryl, heteroarylene-heteroaryl, heteroarylene-heteroarylene-heteroaryl, heterocyclylene-aryl, heterocyclylene-heteroaryl, heteroarylene-NC(O)-heteroaryl, heteroarylene-N═N-aryl, or arylene-(arylalkyl)-OC(O)-hetercyclyl-C(O)C₁₋₆alkylene-aryl, each of which is optionally substituted with 1, 2, 3, or 4 groups independently selected from halogen, C₁₋₆ alkyl, O—C₁₋₆ alkyl, NH₂, NH(C₁₋₆ alkyl), N(C₁₋₆ alkyl)₂, —(CH₂)₁₋₄C(O)NH₂, —(CH₂)₁₋₄C(O)NH(C₁₋₆ alkyl), or —(CH₂)₁₋₄C(O)N(C₁₋₆ alkyl)₂.
51. The compound of any one of embodiments 30-40 and 50, wherein A is

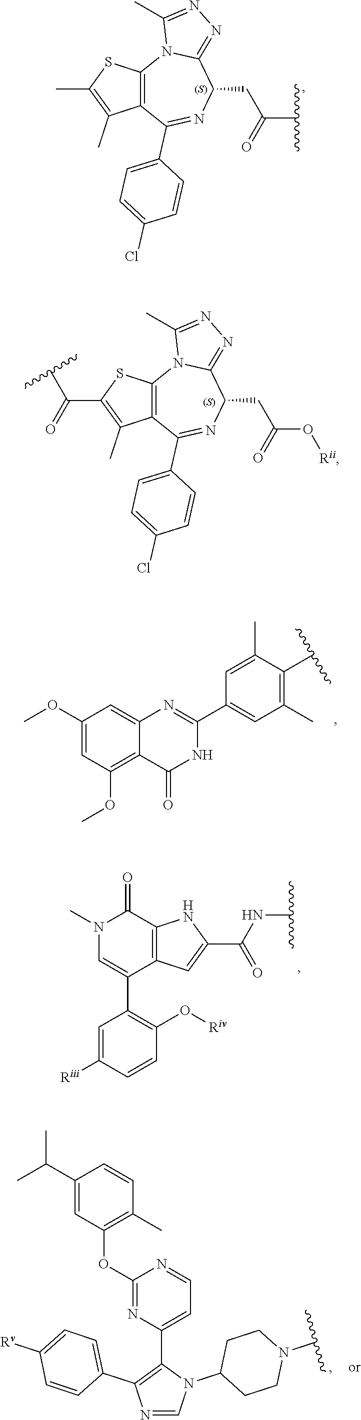

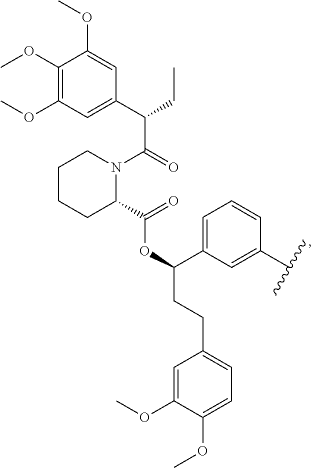

wherein:
Rⁱⁱ is C₁₋₆ alkyl;
Rⁱⁱⁱ is H

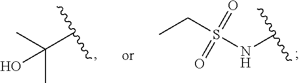

R^{iv} is

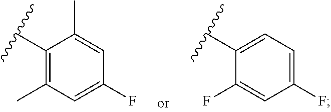

and
   R^{v} is H, F, CF₃, or C₁₋₆ alkyl.
   52. The compound of any one of embodiments 1-51, or a pharmaceutically acceptable salt, a stereoisomer, or a deuterated form thereof, wherein each R¹ is independent halogen.
   53. The compound of embodiment 52, or a pharmaceutically acceptable salt, a stereoisomer, or a deuterated form thereof, wherein each R¹ is —F or —Cl.
   54. The compound of any one of embodiments 1-51, or a pharmaceutically acceptable salt, a stereoisomer, or a deuterated form thereof, wherein each R¹ is independently C₁₋₆ alkyl.
   55. The compound of embodiment 54, or a pharmaceutically acceptable salt, a stereoisomer, or a deuterated form thereof, wherein each R¹ is independently C₁₋₃ alkyl.
   56. The compound of embodiment 54 or 55, or a pharmaceutically acceptable salt, a stereoisomer, or a deuterated form thereof, wherein each R¹ is methyl.
   57. The compound of any one of embodiments 1-56, or a pharmaceutically acceptable salt, a stereoisomer, or a deuterated form thereof, wherein a is 0 or 1.
   58. The compound of any one of embodiments 1-57, or a pharmaceutically acceptable salt, a stereoisomer, or a deuterated form thereof, wherein each R² is independently C₁₋₆ alkyl.
   59. The compound of embodiment 58, or a pharmaceutically acceptable salt, a stereoisomer, or a deuterated form thereof, wherein each R² is independently C₁₋₃ alkyl.
   60. The compound of embodiment 58 or 59, or a pharmaceutically acceptable salt, a stereoisomer, or a deuterated form thereof, wherein each R² is methyl.
   61. The compound of any one of embodiments 1-60, or a pharmaceutically acceptable salt, a stereoisomer, or a deuterated form thereof, wherein b is 0 or 1.
   62. The compound of any one of embodiments 1-61, or a pharmaceutically acceptable salt, a stereoisomer, or a deuterated form thereof, wherein Q is a absent, C₁₋₄ alkylene, —(C₁₋₃ alkylene)C(O)—, C₃₋₆ cycloalkylene, —(C₃₋₆ cycloalkylene)C(O)—, —C(O)—, or —S(O)₂—.
   63. The compound of embodiment 62, or a pharmaceutically acceptable salt, a stereoisomer, or a deuterated form thereof, wherein Q is a absent.
   64. The compound of embodiment 62, or a pharmaceutically acceptable salt, a stereoisomer, or a deuterated form thereof, wherein Q is —CH₂—, —CH₂CH₂—, —CH(CH₃)—, or —C(CH₃)₂—.
   65. The compound of embodiment 62, or a pharmaceutically acceptable salt, a stereoisomer, or a deuterated form thereof, wherein Q is —CH₂—.
   66. The compound of embodiment 62, or a pharmaceutically acceptable salt, a stereoisomer, or a deuterated form thereof, wherein Q is —CH₂CH₂—.
   67. The compound of embodiment 62, or a pharmaceutically acceptable salt, a stereoisomer, or a deuterated form thereof, wherein Q is —C(O)—.
   68. The compound of embodiment 62, or a pharmaceutically acceptable salt, a stereoisomer, or a deuterated form thereof, wherein Q is —S(O)₂—.
   69. The compound of embodiment 62, or a pharmaceutically acceptable salt, a stereoisomer, or a deuterated form thereof, wherein Q is

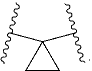 [SMILES: *C1(*)CC1]

70. The compound of embodiment 62, or a pharmaceutically acceptable salt, a stereoisomer, or a deuterated form thereof, wherein Q is —CH₂C(O)—.
71. The compound of embodiment 62, or a pharmaceutically acceptable salt, a stereoisomer, or a deuterated form thereof, wherein Q is

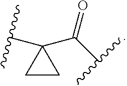 [SMILES: *C(=O)C1(*)CC1]

72. The compound of any one of embodiments 1-71, or a pharmaceutically acceptable salt, a stereoisomer, or a deuterated form thereof, wherein R³ is C₃₋₈ carbocycle, 3-10 membered heterocycle, C₆₋₁₀ aryl, 5-10 membered heteroaryl, or —NR^{X}R^{Y}, wherein the C₃₋₈ carbocycle, 3-8 membered heterocycle, C₆₋₁₀ aryl, and 5-10 membered heteroaryl are each optionally substituted with 1 or 2 R⁴, and each R⁴ is independently halogen, —OH, C₁₋₆ alkyl, C₁₋₆ alkoxy, —C(O)—NR^{X}R^{Y}, or —C(O)O—C₁₋₆ alkyl, and wherein R^{X} is H, C₁₋₆ alkyl, C₁₋₆ haloalkyl, or C₁₋₆ hydroxyalkyl, and R^{Y} is C₁₋₆ alkyl, C₃₋₈ cycloalkyl, C₃₋₈ hydroxycycloalkyl, aryl, or heterocycle.
73. The compound of embodiment 72, or a pharmaceutically acceptable salt, a stereoisomer, or a deuterated form thereof, wherein R³ is C₆₋₁₀ aryl optionally substituted with 1 or 2 R⁴, and wherein each R⁴ is independently halogen, C₁₋₆ alkyl, C₁₋₆ alkoxy, or —C(O)—NR^{X}R^{Y}.
74. The compound of embodiment 73, or a pharmaceutically acceptable salt, a stereoisomer, or a deuterated form thereof, wherein R³ is C₆₋₁₀ aryl optionally substituted with —F, —C₁, C₁₋₃ alkoxy, or —C(O)—NR^{X}R^{Y}, wherein R^{X} is H or C₁₋₆ alkyl, and R^{Y} is C₁₋₆ alkyl.
75. The compound of embodiment 73 or 74, or a pharmaceutically acceptable salt, a stereoisomer, or a deuterated form thereof, wherein R³ is

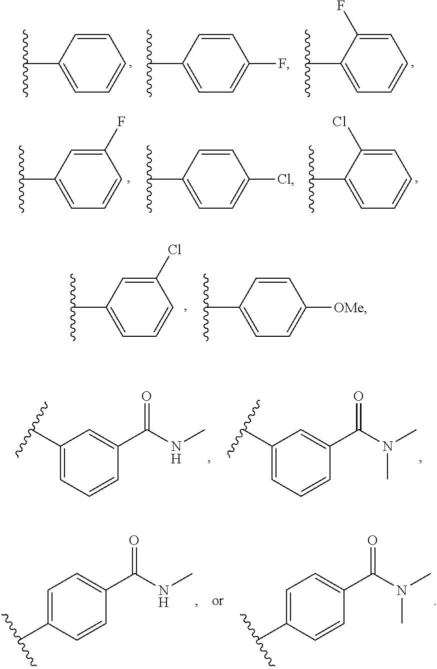

76. The compound of embodiment 72, or a pharmaceutically acceptable salt, a stereoisomer, or a deuterated form thereof, wherein R³ is 5-10 membered heteroaryl optionally substituted with 1 or 2 R⁴; and wherein the heteroaryl contains 1 or 2 heteroatoms selected from N, S, or O.
77. The compound of embodiment 76, or a pharmaceutically acceptable salt, a stereoisomer, or a deuterated form thereof, wherein R³ is

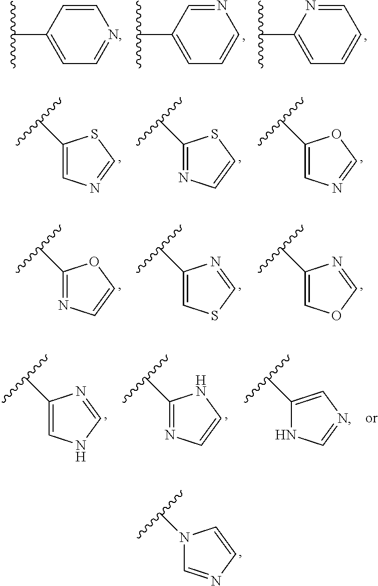

each optionally substituted with one R⁴.
78. The compound of embodiment 76 or 77, or a pharmaceutically acceptable salt, a stereoisomer, or a deuterated form thereof, wherein R³ is

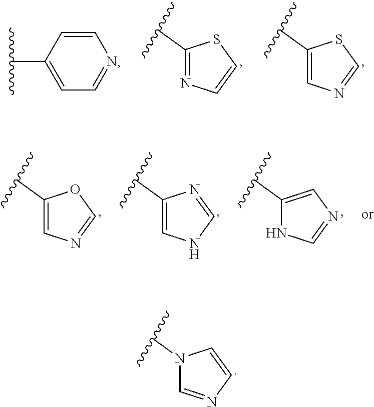

79. The compound of embodiment 72, or a pharmaceutically acceptable salt, a stereoisomer, or a deuterated form thereof, wherein R³ is C₃₋₈ carbocycle optionally substituted with 1 or 2 R⁴, and wherein each R⁴ is independently halogen, —OH, C₁₋₆ alkyl, or C₁₋₆ alkoxy.
80. The compound of embodiment 79, or a pharmaceutically acceptable salt, a stereoisomer, or a deuterated form thereof, wherein R³ is

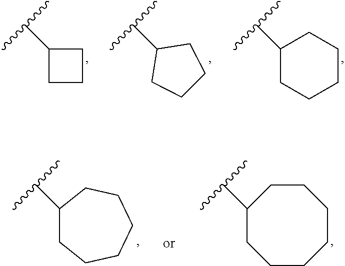 [SMILES: *C1CCC1 ; *C1CCCC1 ; *C1CCCCC1 ; *C1CCCCCC1 ; *C1CCCCCCC1]

or each optionally substituted with one R⁴.
81. The compound of embodiment 72, or a pharmaceutically acceptable salt, a stereoisomer, or a deuterated form thereof, wherein R³ is 3-10 membered heterocycle containing 1 or 2 heteroatoms selected from N, O, or S, and the heterocycle is optionally substituted with 1 or 2 R⁴, and wherein each R⁴ is independently —OH, C₁₋₆ alkyl, or —C(O)O—(C₁₋₆ alkyl).
82. The compound of embodiment 81, or a pharmaceutically acceptable salt, a stereoisomer, or a deuterated form thereof, wherein each R⁴ is independently —OH, C₁₋₃ alkyl, or —C(O)O—(C₁₋₅ alkyl).
83. The compound of embodiment 81 or 82, or a pharmaceutically acceptable salt, a stereoisomer, or a deuterated form thereof, wherein R³ is

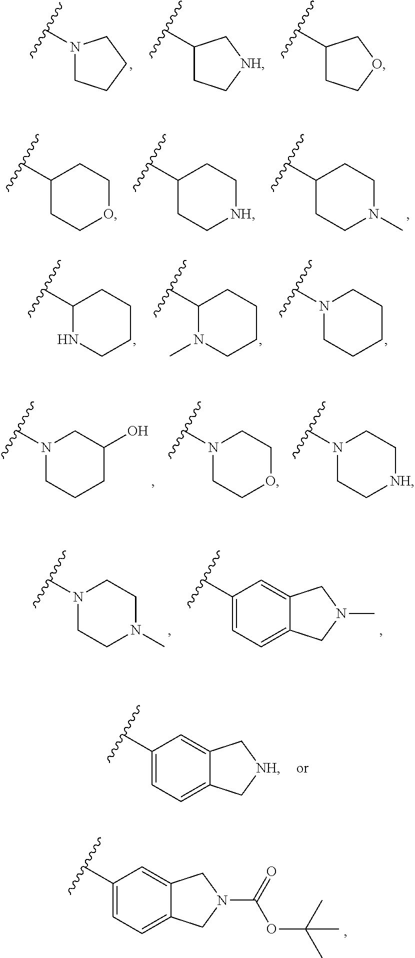

each optionally substituted with 1 or 2 R⁴.
84. The compound of any one of embodiments 81-83, or a pharmaceutically acceptable salt, a stereoisomer, or a deuterated form thereof, wherein R³ is

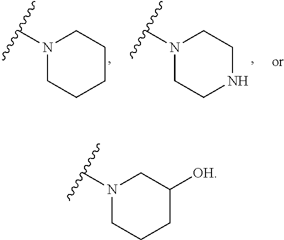

85. The compound of embodiment 72, or a pharmaceutically acceptable salt, a stereoisomer, or a deuterated form thereof, wherein R³ is —NR^{X}R^{Y}, and wherein R^{X} is H, C₁₋₆ alkyl, C₁₋₆ haloalkyl, or C₁₋₆ hydroxyalkyl, and R^{Y} is C₁₋₆ alkyl, C₃₋₈ cycloalkyl, C₃₋₈ hydroxycycloalkyl, aryl, or heterocycle.
86. The compound of embodiment 85, or a pharmaceutically acceptable salt, a stereoisomer, or a deuterated form thereof, wherein R^{X} is H, or methyl, and R, is methyl, —CH(CH₃)₂

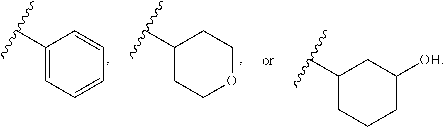

87. The compound of embodiment 85 or 86, or a pharmaceutically acceptable salt, a stereoisomer, or a deuterated form thereof, wherein R³ is

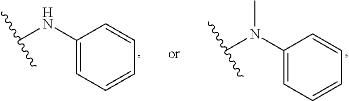

NHCH(CH₃)₂, —N(CH₃)₂, —NH(CH₃),

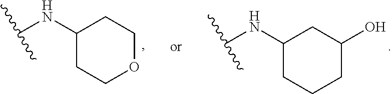 [SMILES: *NC1CCOCC1 ; *NC1CCCC(O)C1]

88. The compound of any one of embodiments 1-63 and 72-87, or a pharmaceutically acceptable salt, a stereoisomer, or a deuterated form thereof, wherein Q is absent, and R³ is

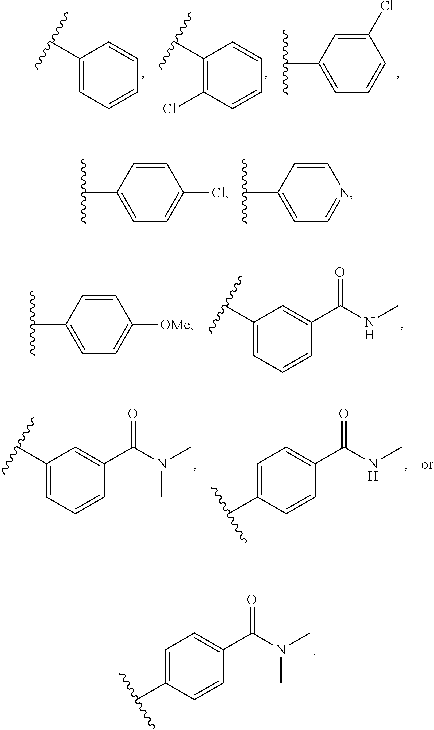

89. The compound of any one of embodiments 1-72, or a pharmaceutically acceptable salt, a stereoisomer, or a deuterated form thereof, wherein -Q-R³ is

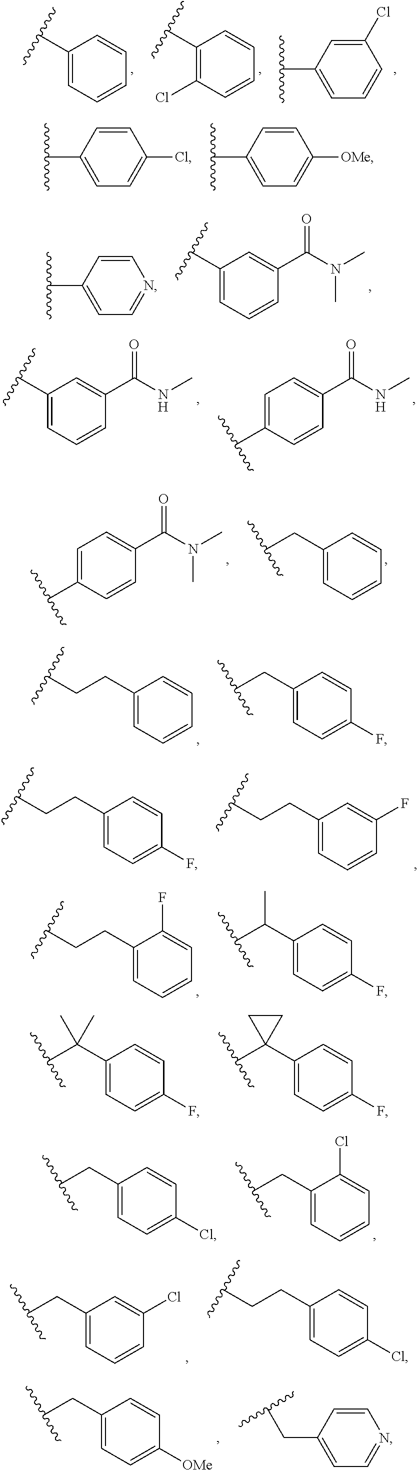

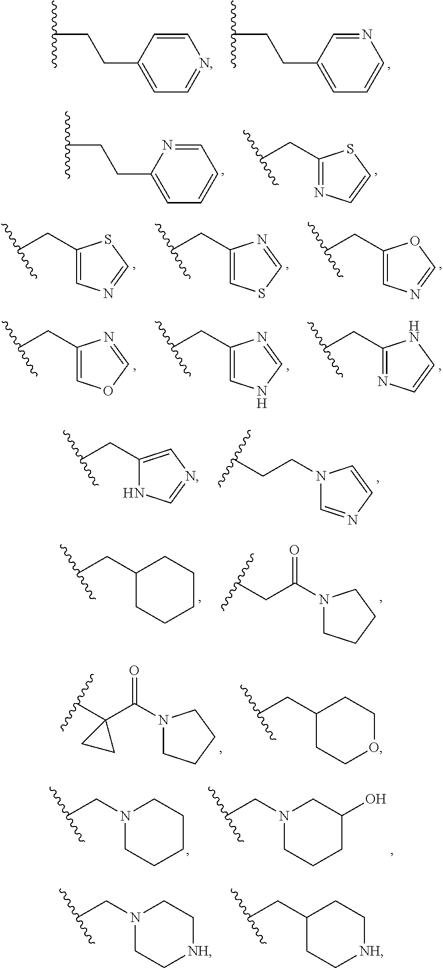

—CH₂C(O)—N(CH₃)₂, —CH₂C(O)—NH(CH₃)

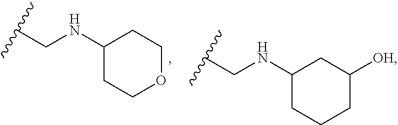

or —CH₂CH₂—NH—CH(CH₃)₂.
90. The compound of any one of embodiments 1-72, or a pharmaceutically acceptable salt, a stereoisomer, or a deuterated form thereof, wherein -Q-R³ is

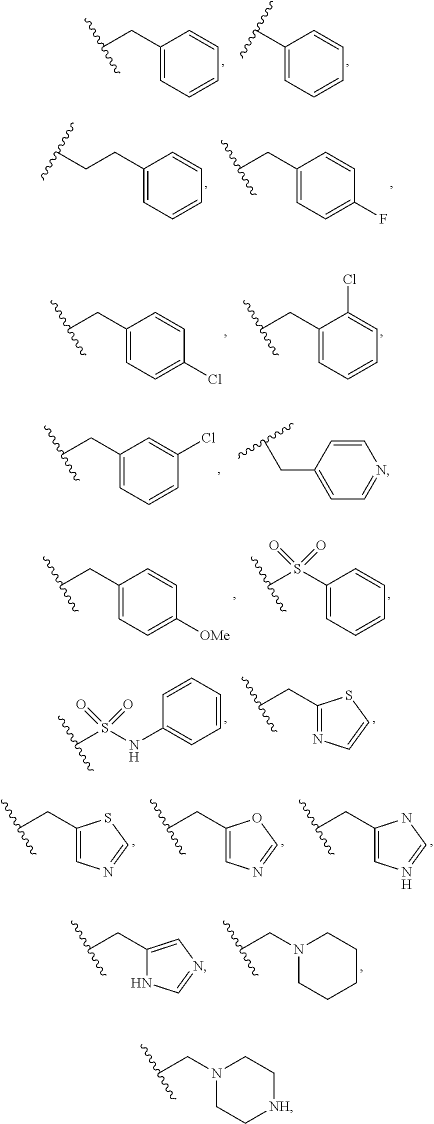

—CH₂CH₂—NH—CH(CH₃)₂,

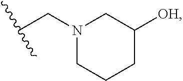 [SMILES: *CN1CCCC(O)C1]

—CH₂C(O)—N(CH₃)₂, —CH₂C(O)—NH(CH₃),

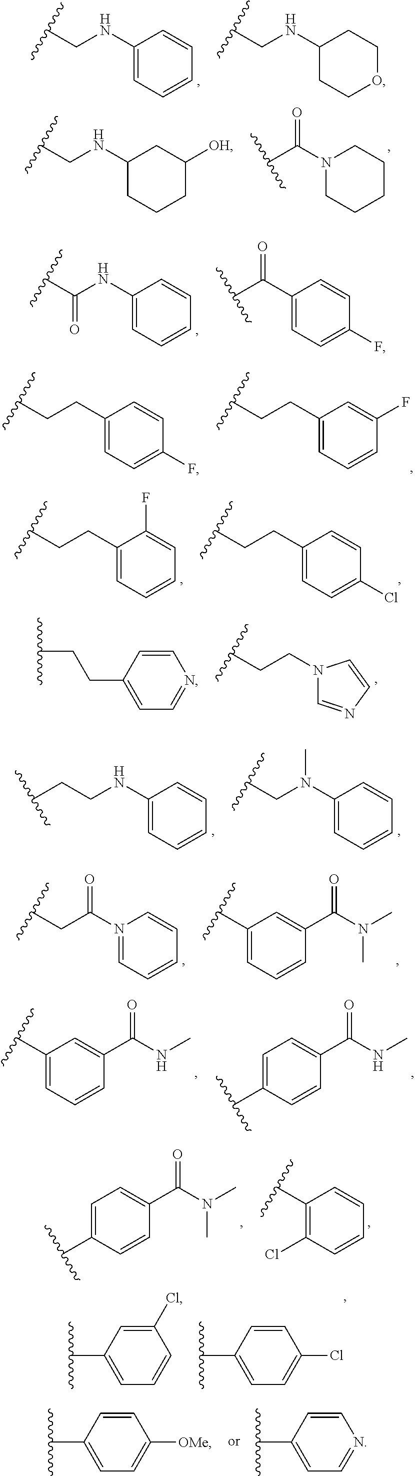

91. The compound of any one of embodiments 1-72 and 89-90, or a pharmaceutically acceptable salt, a stereoisomer, or a deuterated form thereof, wherein -Q-R³ is

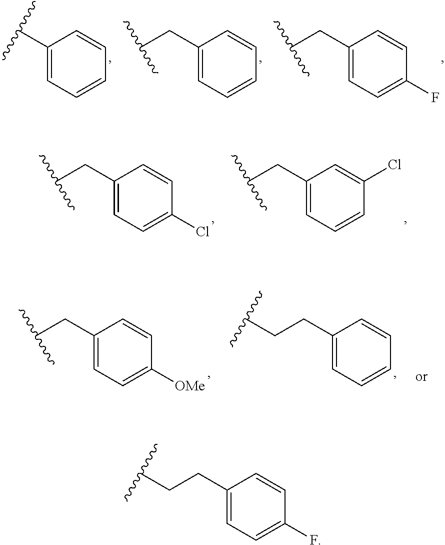

92. The compound of any one of embodiments 1, 2, and 5, wherein the compound of formula (I) is:

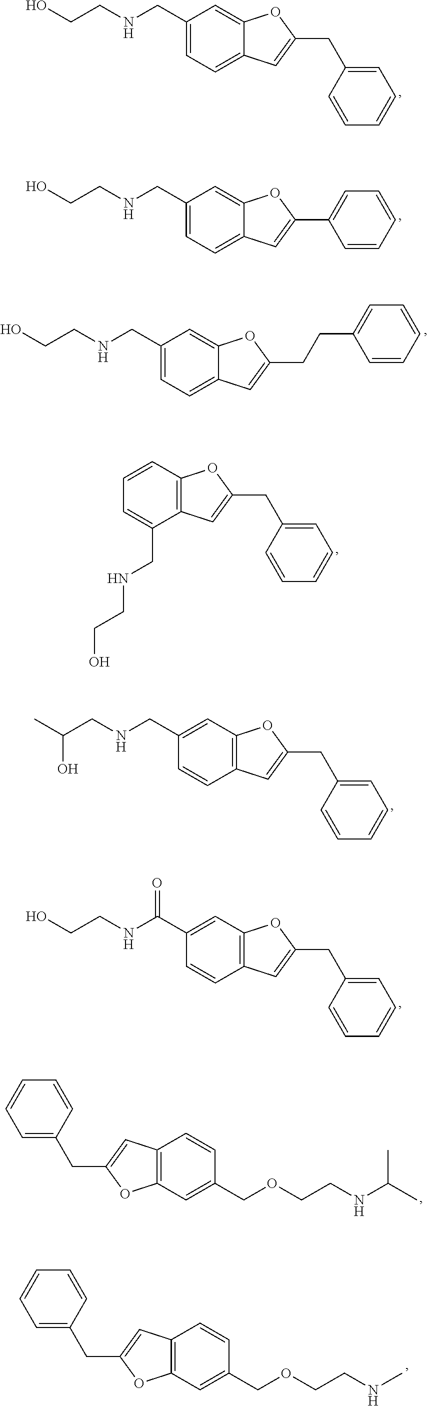

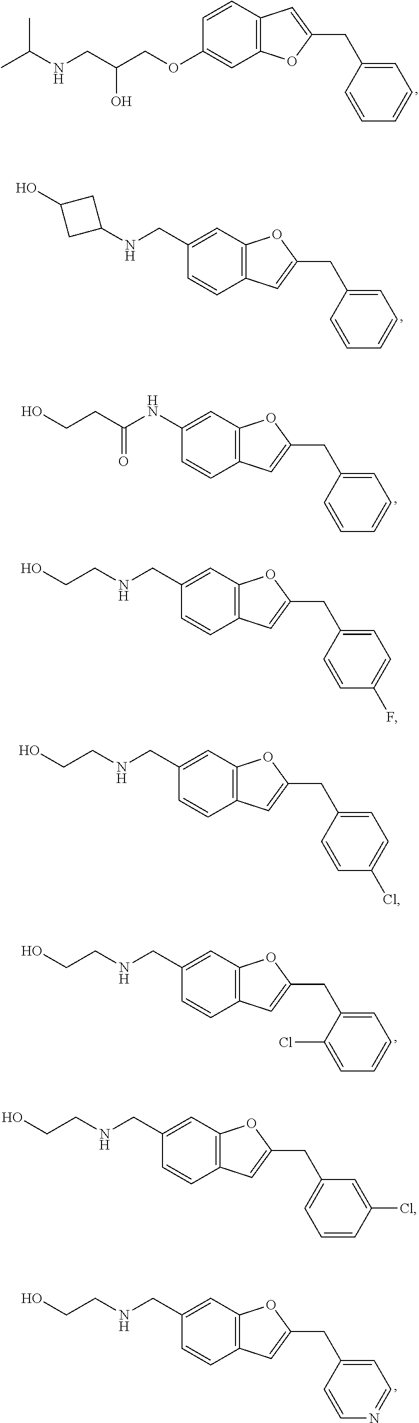

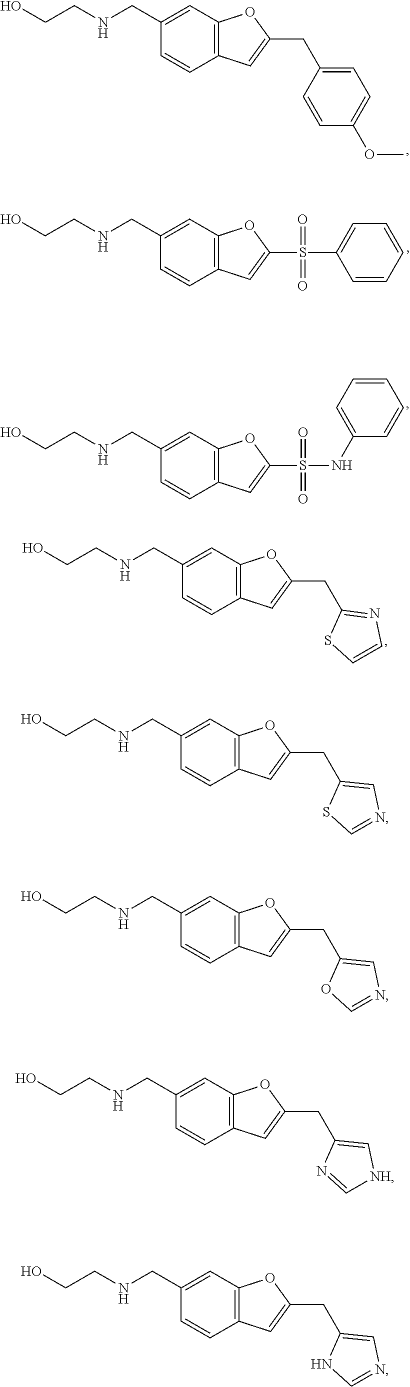

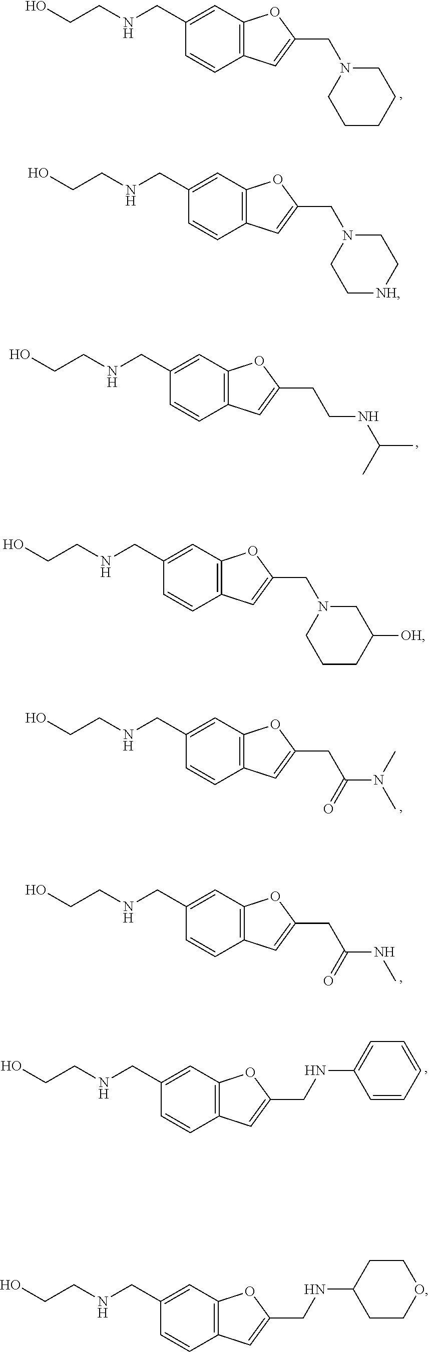

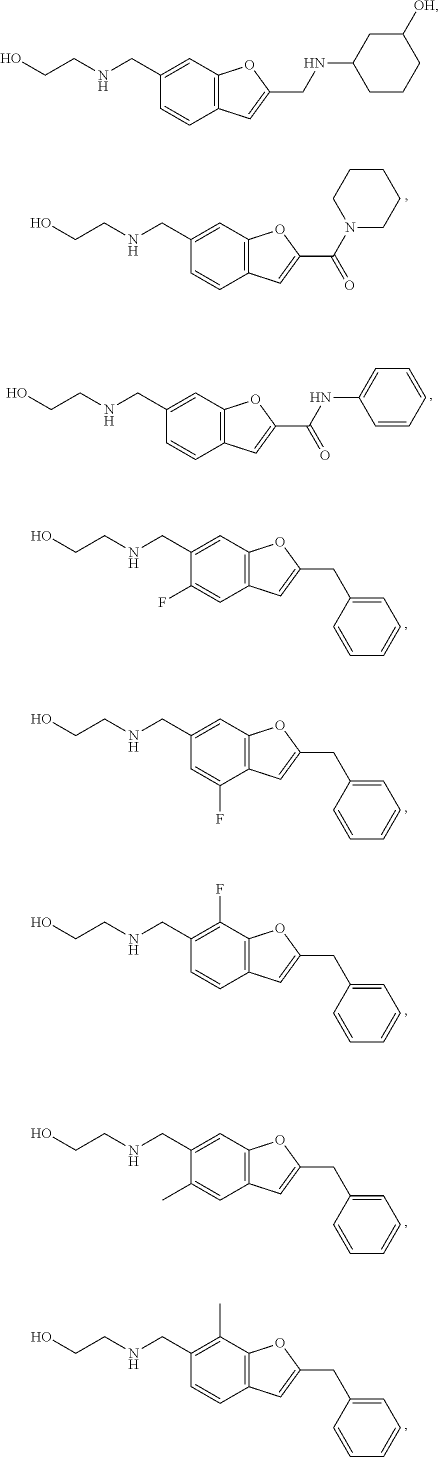

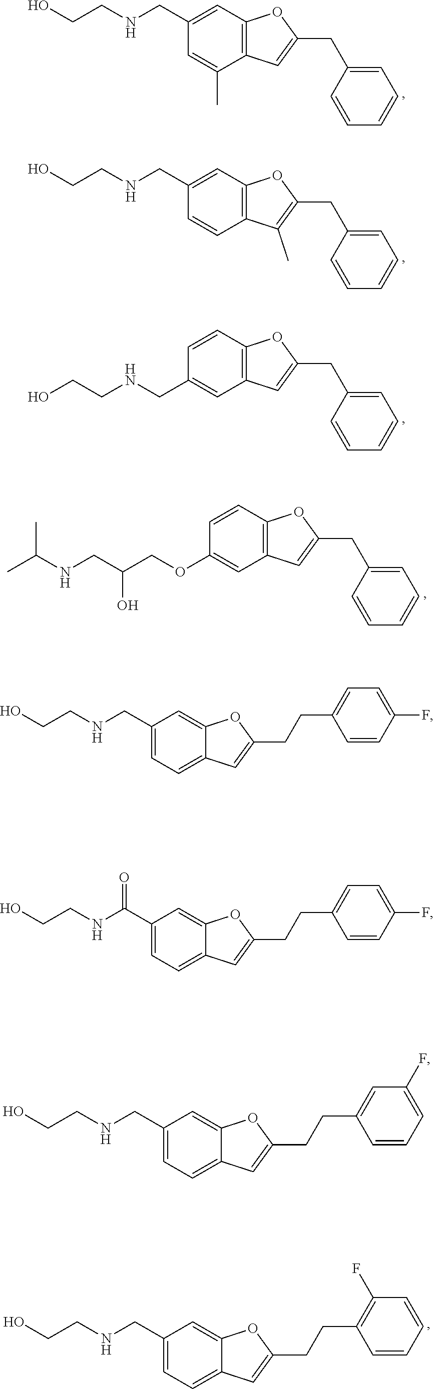

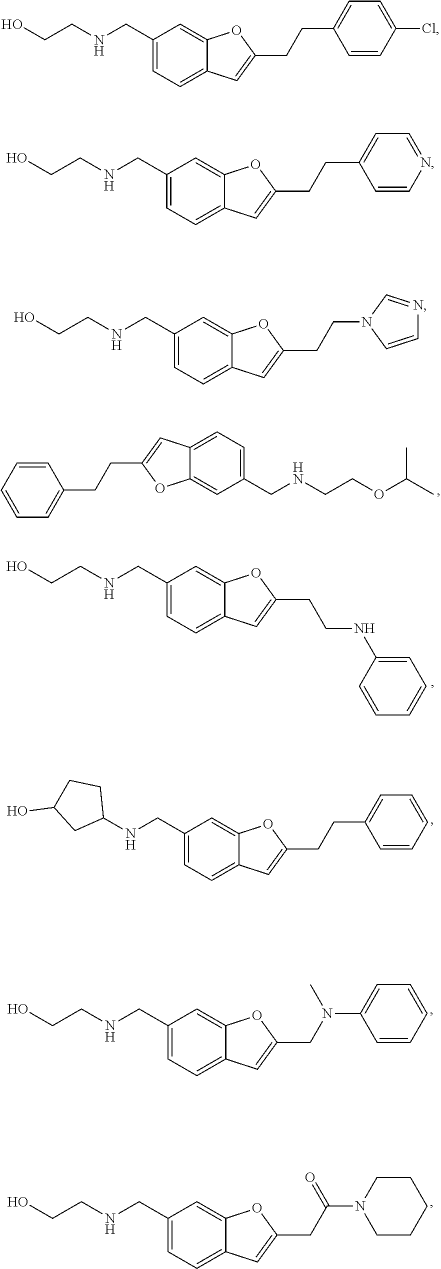

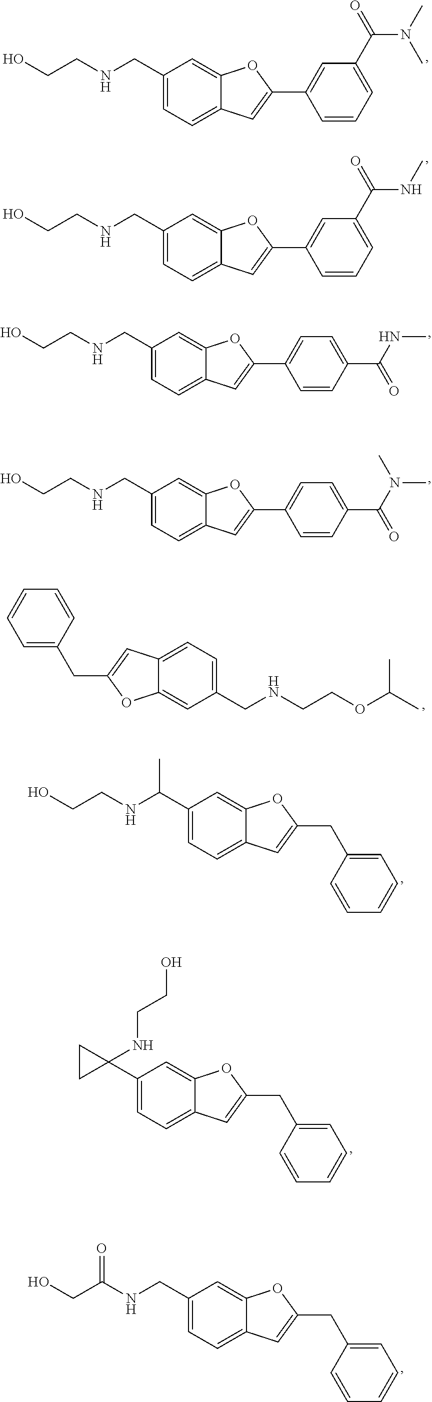

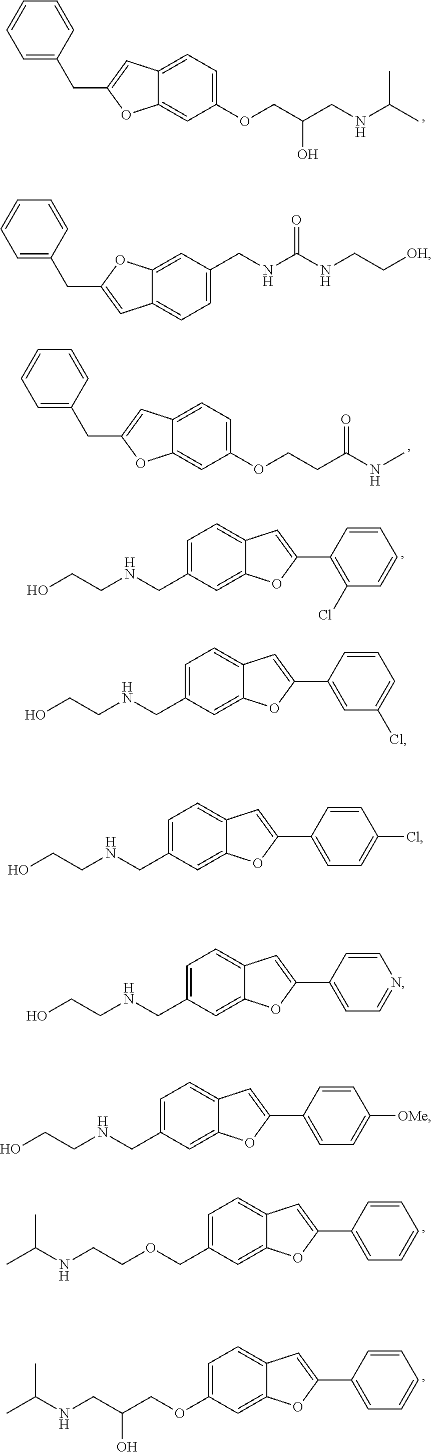

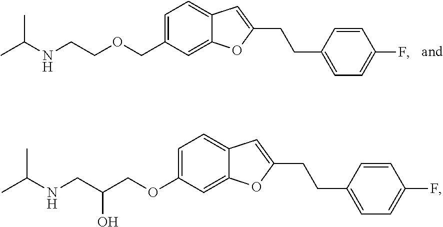 [SMILES: CC(C)NCCOCc1ccc2cc(CCc3ccc(F)cc3)oc2c1 ; CC(C)NCC(O)COc1ccc2cc(CCc3ccc(F)cc3)oc2c1]

or a Pharmaceutically acceptable salt, a stereoisomer, or deuterated form thereof.
93. The compound of any one of embodiments 1, 3, and 6, wherein the compound of formula (I) is:

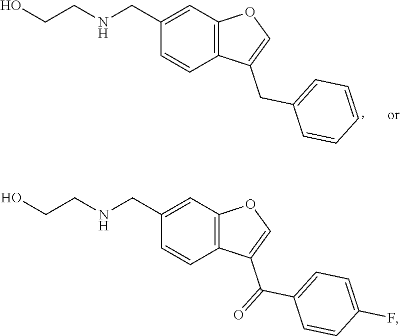 [SMILES: OCCNCc1ccc2c(Cc3ccccc3)coc2c1 ; O=C(c1ccc(F)cc1)c1coc2cc(CNCCO)ccc12]

or a pharmaceutically acceptable salt or deuterated form thereof.
94. The compound of any one of embodiments 1, 4, and 7, wherein the compound of formula (I) is:

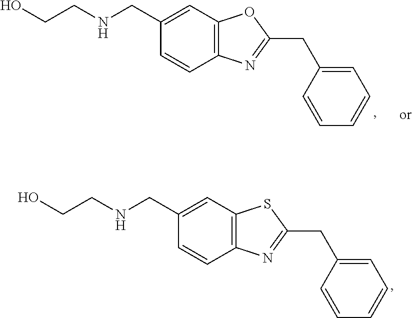 [SMILES: OCCNCc1ccc2nc(Cc3ccccc3)oc2c1 ; OCCNCc1ccc2nc(Cc3ccccc3)sc2c1]

or a pharmaceutically acceptable salt or deuterated form thereof.
95. The compound of embodiment 15, wherein the compound of formula (II) is

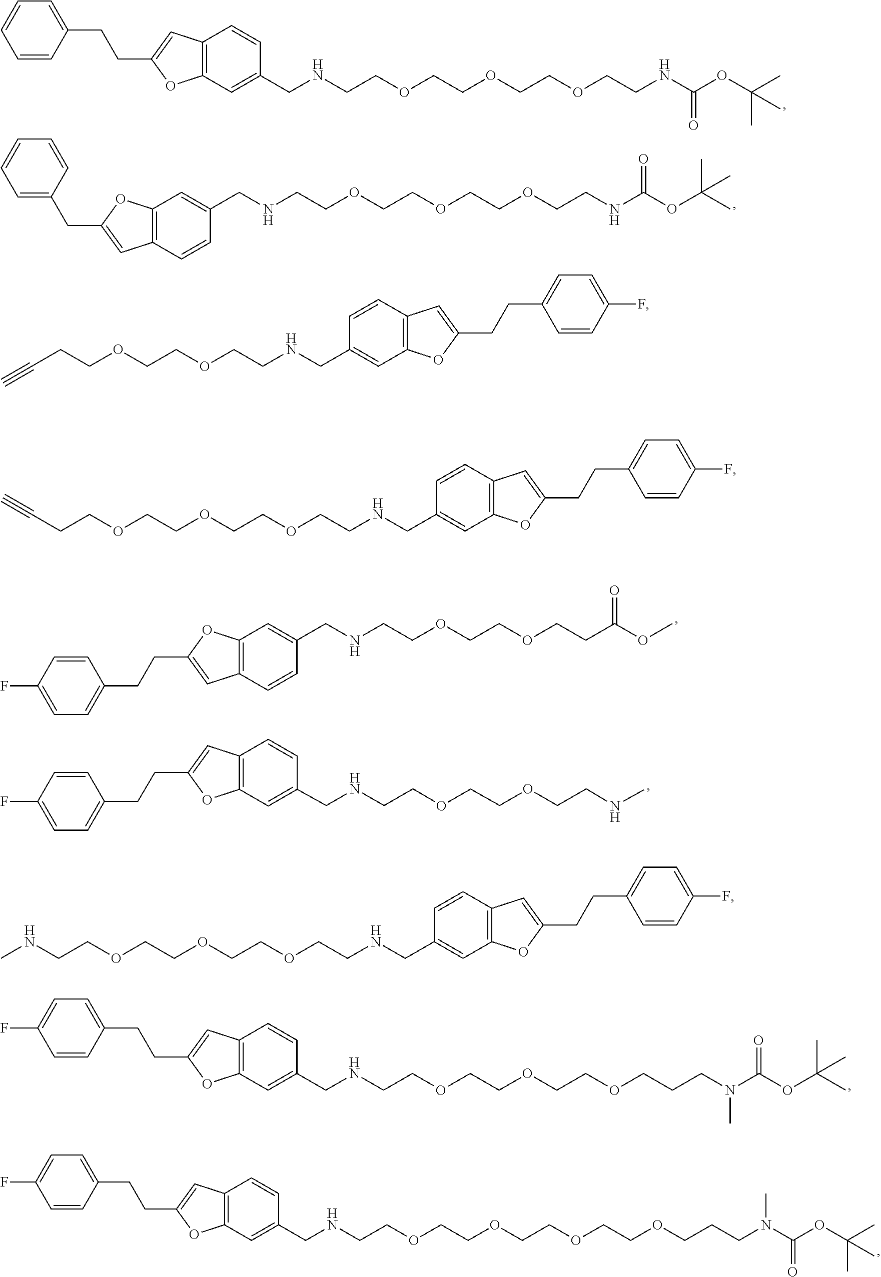

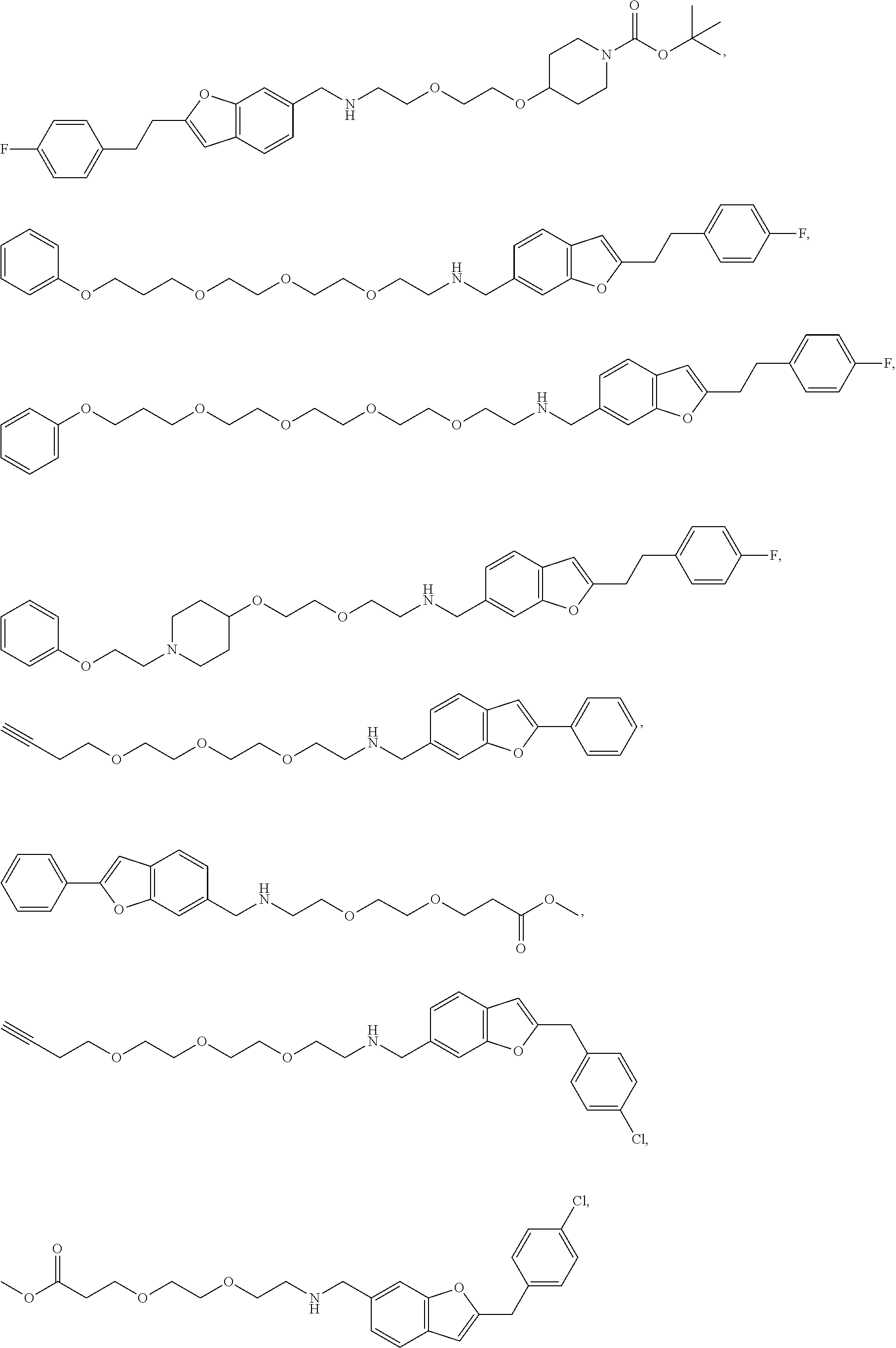

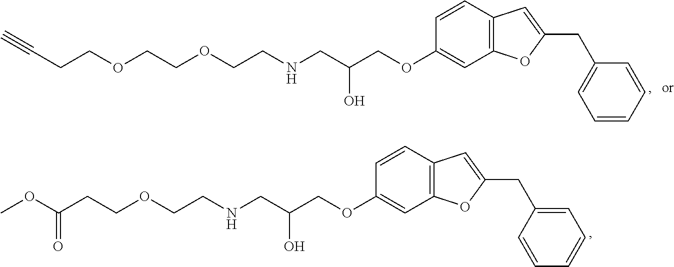 [SMILES: C#CCCOCCOCCNCC(O)COc1ccc2cc(Cc3ccccc3)oc2c1 ; COC(=O)CCOCCNCC(O)COc1ccc2cc(Cc3ccccc3)oc2c1]

or a pharmaceutically acceptable salt, a stereoisomer, or deuterated form thereof.
96. The compound of embodiment 30, wherein the compound of formula (III) is

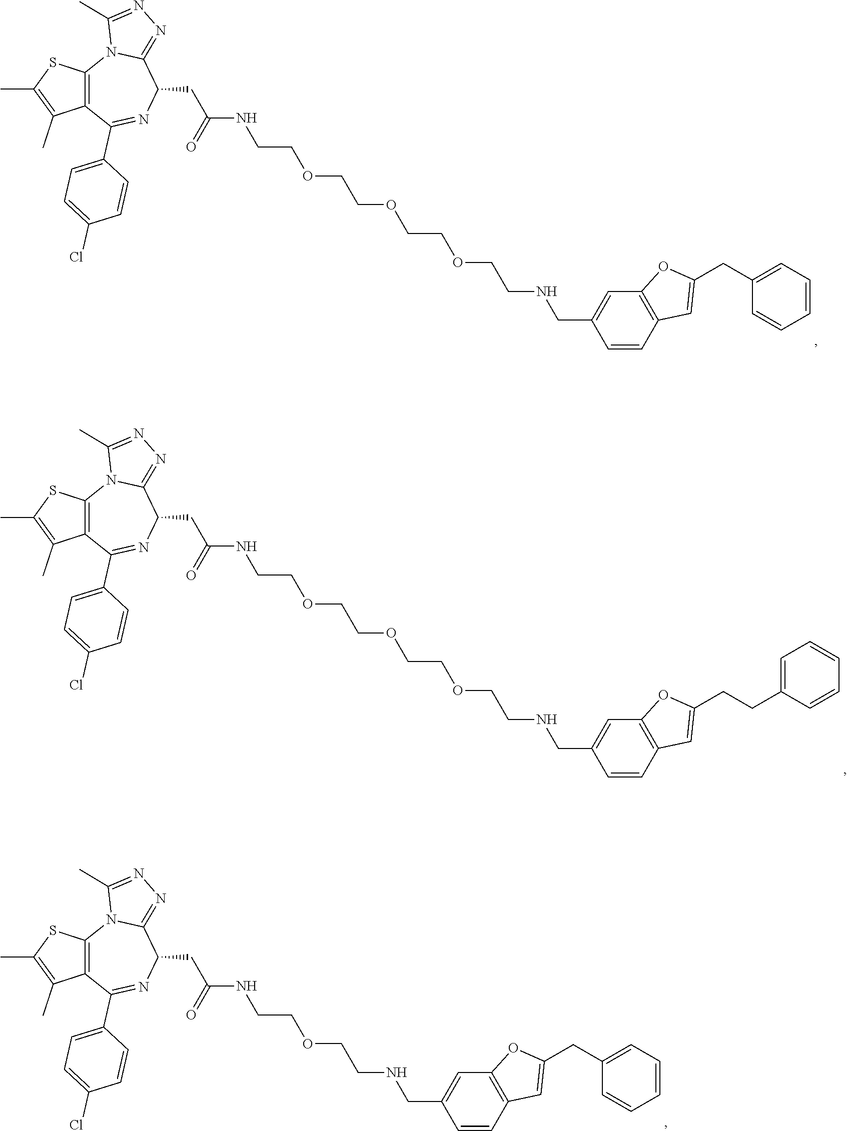 [SMILES: Cc1sc2c(c1C)C(c1ccc(Cl)cc1)=N[C@@H](CC(=O)NCCOCCOCCOCCNCc1ccc3cc(Cc4ccccc4)oc3c1)c1nnc(C)n1-2 ; Cc1sc2c(c1C)C(c1ccc(Cl)cc1)=N[C@@H](CC(=O)NCCOCCOCCOCCNCc1ccc3cc(CCc4ccccc4)oc3c1)c1nnc(C)n1-2 ; Cc1sc2c(c1C)C(c1ccc(Cl)cc1)=N[C@@H](CC(=O)NCCOCCNCc1ccc3cc(Cc4ccccc4)oc3c1)c1nnc(C)n1-2]

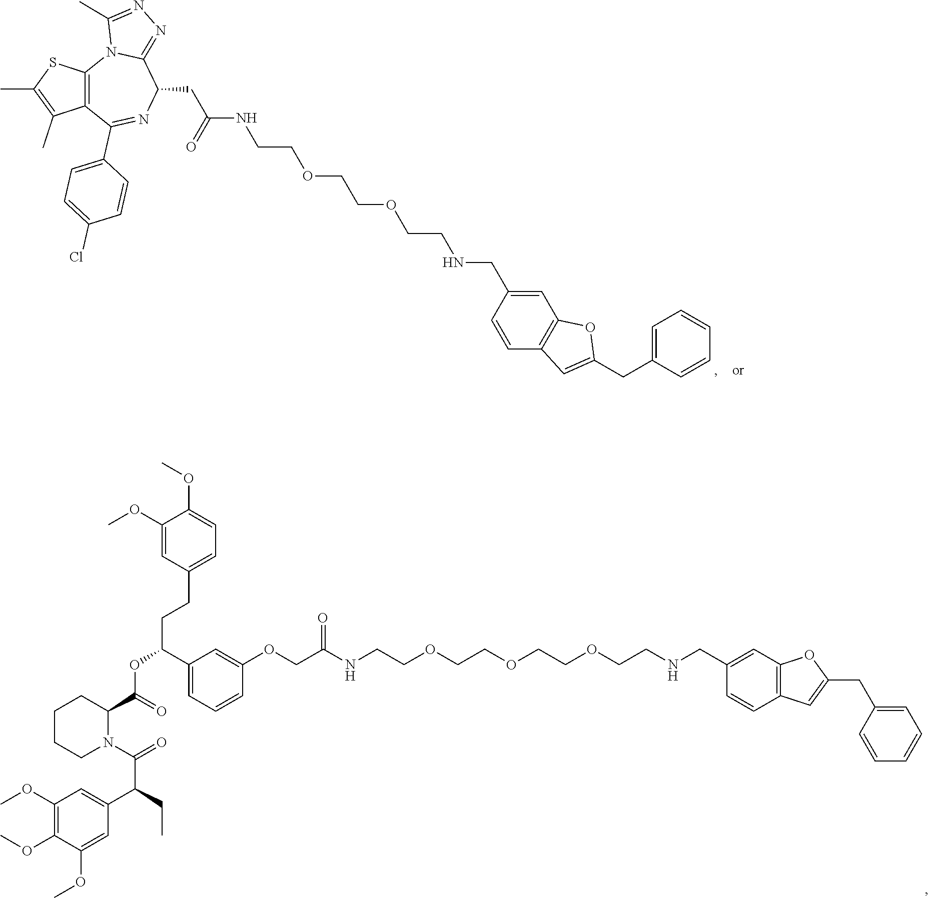 [SMILES: Cc1sc2c(c1C)C(c1ccc(Cl)cc1)=N[C@@H](CC(=O)NCCOCCOCCNCc1ccc3cc(Cc4ccccc4)oc3c1)c1nnc(C)n1-2 ; CC[C@H](C(=O)N1CCCC[C@H]1C(=O)O[C@H](CCc1ccc(OC)c(OC)c1)c1cccc(OCC(=O)NCCOCCOCCOCCNCc2ccc3cc(Cc4ccccc4)oc3c2)c1)c1cc(OC)c(OC)c(OC)c1]

or a pharmaceutically acceptable salt, or deuterated form thereof.
97. A method of modulating autophagy in a subject, comprising administering to the subject a compound of any one of the preceding claims.
98. The method of embodiment 97, wherein the compound contacts p62.
99. The method of embodiment 98, wherein the compound increases activity of p62, thereby causing autophagy.
100. The method of embodiment 98, wherein the compound decreases activity of p62, thereby reducing autophagy.
101. A method of degrading a target protein in a subject in need thereof, comprising administering a compound of any one of the preceding embodiments.
102. A method of inducing autophagy in a subject in need thereof, comprising administering a compound of any one of the preceding embodiments.
103. The method of embodiment 102, wherein the compound contacts p62, thereby inducing autophagy.

### EXAMPLES

The present disclosure is further illustrated by reference to the following Examples. However, it should be noted that these Examples, like the embodiments described above, are illustrative and are not to be construed as restricting the scope of the disclosure in any way.

In embodiments, compounds of the present disclosure can be synthesized using the following methods. General reaction conditions are given, and reaction products can be purified by generally known methods including silica gel chromatography using various organic solvents such as hexane, dichloromethane, ethyl acetate, methanol and the like or preparative reverse phase high pressure liquid chromatography.

Abbreviations

| | |
|---|---|
| equiv. | equivalence |
| h | hour(s) |
| min | minutes |
| THF | tetrahydrofuran |
| RT | room temperature |
| TLC | thin layer chromatography |
| EtOAc | ethyl acetate |
| Et₂O | diethylether |
| ACN | acetonitrile |
| EDC•HCl | (1-ethyl-3-(3-dimethylaminopropyl)carbodiimide |
| | hydrochloride) |
| HOBt | 1-hydroxybenzotriazole |
| FA | formic acid |
| TFA | trifluoroacetic acid |
| aq. | aqueous |
| br. | broad |
| sat. | saturated |
| DIBAL-H | diisobutylaluminum hydride |
| DMP | Dess-Martin periodinane |
| DIPEA | N,N-Diisopropylethylamine |
| HATU | Hexafluorophosphate Azabenzotriazole Tetramethyl Uronium |
| NBS | N-bromosuccinimide |
| (BzO)₂ | Benzoyl peroxide |
| NIS | N-Iodosuccinimide |
| (Boc)₂O | Di-tert-butyl decarbonate |
| Bn | Benzyl |
| DCDMH | 2,4-Dichloro-5,5-dimethylhydantoin |
| IBCF | Isobutyl chloroformate |
| Ts | Tosyl |
| dppp | 1,3-Bis(diphenylphosphino)propane |
| TBAB | tetrabutylammonium bromide |
| DMA | dimethylacetamide |
| Fmoc-OSu | 9-Fluorenylmethyl N-succinimidyl carbonate |

### Example 1: Synthesis of Synthesis of Compound 2 Via Synthesis Route A

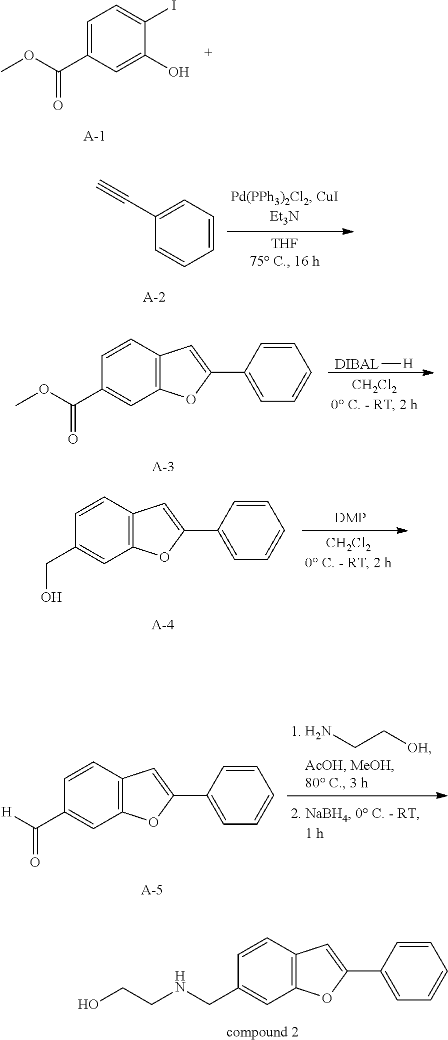

### Synthesis of methyl 2-phenylbenzofuran-6-carboxylate (A-3)

In a sealed tube, to a degassed solution of methyl 3-hydroxy-4-iodobenzoate A-1 (1.00 g, 3.60 mmol, 1.0 equiv.) in THE (10 mL) at RT, was added Et₃N (2.51 mL, 18.0 mmol, 5.0 equiv.), ethynylbenzene A-2 (0.40 mL, 3.60 mmol, 1.0 equiv.), CuI (68.6 mg, 0.36 mmol, 0.1 equiv.) and PdCl₂(PPh₃)₂(126 mg, 0.18 mmol, 0.05 equiv.). The resultant mixture was stirred at 75° C. for 16 h. Upon the completion of reaction by TLC, the reaction mixture was diluted with water (80 mL) and extracted with EtOAc (2×100 mL). The combined organic layers were dried over anhydrous Na₂SO₄, filtered, evaporated, and purified by silica gel column (60-120 mesh) chromatography (elution with 10% EtOAc/hexane) to afford A-3 as a brown solid (485 mg, yield: 53%). TLC system: EtOAc/hexane (30:70), R_{f} value=0.4; ¹H NMR (400 MHz, CDCl₃) δ ppm: 8.22-8.21 (br. s, 1H), 7.95 (dd, J=8.0 Hz, 1.2 Hz, 1H), 7.90-7.88 (m, 2H), 7.60 (d, J=8.0 Hz, 1H), 7.50-7.45 (m, 2H), 7.42-7.39 (m, 1H), 7.06 (s, 1H), 3.96 (s, 3H).

### Synthesis of (2-phenylbenzofuran-6-yl)methanol (A-4)

To a stirred solution of A-3 (300 mg, 1.19 mmol, 1.0 equiv.) in CH₂Cl₂ (6 mL) at 0° C., was added 1M DIBAL-H in toluene (4.76 mL, 4.76 mmol, 4.0 equiv.). The resultant mixture was allowed to warm to RT and stirred at RT for 2 h. Upon the completion of reaction by TLC, the reaction mixture was quenched with NH₄Cl solution (30 mL) and extracted with CH₂Cl₂(2×30 mL). The combined organic layers were dried over anhydrous Na₂SO₄, filtered, and concentrated under reduced pressure. The residue obtained was triturated with n-pentane (20 mL), Et₂O (20 mL), and dried under vacuum to afford A-4 as off-white solid (250 mg, yield: 94%). TLC system: EtOAc/hexane (30:70), R_{f} value=0.4; ¹H NMR (400 MHz, CDCl₃) δ ppm: 7.88-7.86 (m, 2H), 7.57-7.55 (m, 2H), 7.48-7.44 (m, 2H), 7.37-7.34 (m, 1H), 7.25-7.23 (m, 1H), 7.01 (s, 1H), 4.81 (d, J=6.0 Hz, 2H), 1.70 (t, J=6.0 Hz, 1H).

### Synthesis of 2-phenylbenzofuran-6-carbaldehyde (A-5)

To a stirred solution of A-4 (250 mg, 1.11 mmol, 1.0 equiv.) in CH₂Cl₂ (10 mL) at 0° C., was added Dess-Martin periodinane (708 mg, 1.67 mmol, 1.5 equiv.). The resultant mixture was stirred at RT for 2 h. Upon the completion of reaction by TLC, the reaction mixture was diluted with water (30 mL) and extracted with CH₂Cl₂ (2×30 mL). The combined organic layers were dried over anhydrous Na₂SO₄, filtered, and concentrated under reduced pressure. The crude product obtained was purified by silica gel column (60-120 mesh) chromatography to afford A-5 as an off-white solid (225 mg, yield: 91%). TLC system: EtOAc/hexane (20:80), R_{f} value=0.5; ¹H NMR (400 MHz, CDCl₃) δ ppm: 10.07 (s, 1H), 8.03 (br. s, 1H), 7.93-7.90 (m, 2H), 7.79 (dd, J=8.0 Hz, 1.2 Hz, 1H), 7.70 (d, J=8.0 Hz, 1H), 7.52-7.47 (m, 2H), 7.45-7.41 (m, 1H), 7.10 (d, J=1.2 Hz, 1H).

### Synthesis of 2-(((2-phenylbenzofuran-6-yl)methyl)amino)ethan-1-ol as Formic Acid Salt (Compound 2)

To a stirred solution of A-5 (225 mg, 1.01 mmol, 1.0 equiv.) in MeOH (7 mL) at RT, was added 2-aminoethan-1-ol (124 mg, 2.02 mmol, 2.0 equiv.) and AcOH (6 μL, 0.1 mmol, 0.1 equiv.). The reaction mixture was stirred at 80° C. for 3 h before the mixture was cooled to 0° C. and NaBH₄ (76.4 mg, 2.02 mmol, 2.0 equiv.) was added portion-wise. The resultant mixture was stirred at RT for 1 h and upon the completion of reaction by TLC, reaction mixture was concentrated under reduced pressure and purified by reverse phase column chromatography (Grace column, elution with 30% MeCN/0.02% FA in H₂O) to afford compound 2 (formic acid salt) as a white solid (30 mg, yield: 9%). TLC system: MeOH/CH₂Cl₂(10:90), R_{f} value=0.1; ¹H NMR (400 MHz, DMSO-d₆) δ ppm: 8.31 (br. s, 1H; HCOOH), 7.93-7.90 (m, 2H), 7.62 (s, 1H), 7.59 (d, J=8.0 Hz, 1H), 7.53-7.49 (m, 2H), 7.43-7.38 (m, 2H), 7.26 (dd, J=8.0 Hz, 1.2 Hz, 1H), 3.89 (s, 2H), 3.50 (t, J=6.0 Hz, 2H), 2.63 (t, J=6.0 Hz, 2H); LCMS (ESI) m/z 268.1 [C₁₇H₁₇NO₂+H]⁺.

### Example 2: Synthesis of Compound 6 Via Synthesis Route A

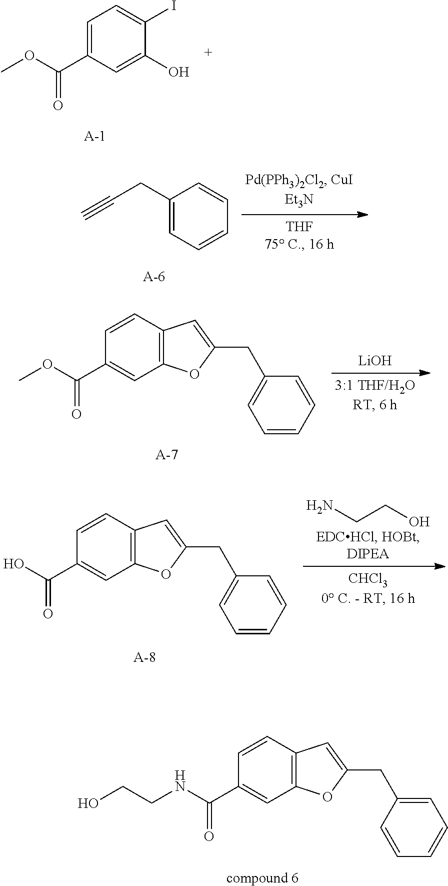

### Synthesis of methyl 2-benzylbenzofuran-6-carboxylate (A-7)

Compound A-7 was obtained following the Pd-catalyzed benzofuran ring formation procedure similar to that for the synthesis of compound A-3, utilizing prop-2-yn-1-ylbenzene A-6 (1.0 equiv.) and reaction conditions of 75° C. for 16 h. Compound A-7 was obtained in 50% yield as yellow liquid following silica gel (60-120 mesh) column chromatography (elution with 10% EtOAc/hexane). TLC system: EtOAc/hexane (30:70), R_{f} value=0.5; ¹H NMR (400 MHz, CDCl₃) δ ppm: 8.10 (s, 1H), 7.90 (dd, J=8.0 Hz, 1.2 Hz, 1H), 7.48 (d, J=8.0 Hz, 1H), 7.36-7.27 (m, 5H), 6.40 (s, 1H), 4.13 (s, 2H), 3.92 (s, 3H).

### Synthesis of 2-benzylbenzofuran-6-carboxylic acid (A-8)

To a stirred solution of A-7 (300 mg, 1.13 mmol, 1.0 equiv.) in THE (4.5 mL) at RT, was added LiOH·H₂O (299 mg, 7.12 mmol, 6.3 equiv., dissolved in 1.5 mL of H₂O). The resultant mixture was stirred at RT for 6 h and upon the completion of reaction by TLC, the mixture was diluted with H₂O (30 mL) and extracted with Et₂O (2×30 mL) to remove unreacted starting material and non-polar impurities. The aqueous layer was then acidified with 2N HCl (5 mL) and extracted with 10% MeOH in CH₂Cl₂ (2×50 mL). The combined organic layers were dried over anhydrous Na₂SO₄, filtered, and concentrated under reduced pressure to afford A-8 as yellow solid (230 mg, yield: 81%). TLC system: MeOH/CH₂Cl₂ (5:95), R_{f} value=0.4; ¹H NMR (400 MHz, CDCl₃) δ ppm: 8.18 (s, 1H), 7.97 (dd, J=8.0 Hz, 1.6 Hz, 1H), 7.51 (d, J=8.0 Hz, 1H), 7.37-7.25 (m, 5H), 6.43 (s, 1H), 4.15 (s, 2H).

### Synthesis of 2-benzyl-N-(2-hydroxyethyl)benzofuran-6-carboxamide (compound 6)

To a stirred solution of A-8 (200 mg, 0.79 mmol, 1.0 equiv.) in CHCl₃ (2 mL) at 0° C., was added 2-aminoethan-1-ol (121 mg, 1.98 mmol, 2.5 equiv.), EDC·HCl (228 mg, 1.19 mmol, 1.5 equiv.), HOBt (161 mg, 1.19 mmol, 1.5 equiv.) and DIPEA (0.41 mL, 2.38 mmol, 3.0 equiv.). The resultant mixture was allowed to warm to RT and stirred at RT for 16 h. Upon the completion of reaction by TLC, H₂O (10 mL) was added to the reaction mixture and the mixture was extracted with CH₂Cl₂ (2×50 mL). The combined organic layers were dried over anhydrous Na₂SO₄, filtered, concentrated under reduced pressure, and purified by reverse phase column chromatography (Grace column, elution with 30% MeCN/0.01% FA in H₂O) to afford compound 6 as an off-white solid (25 mg, yield: 11%). TLC system: MeOH/CH₂Cl₂ (10:90), R_{f} value=0.3; melting point range: 84-86° C.; ¹H NMR (400 MHz, DMSO-d₆) δ ppm: 8.40 (t, J=5.6 Hz, 1H), 7.97 (s, 1H), 7.73 (dd, J=8.0 Hz, 1.2 Hz, 1H), 7.59 (d, J=8.4 Hz, 1H), 7.37-7.32 (m, 4H), 7.28-7.25 (m, 1H), 6.66 (s, 1H), 4.71 (t, J=5.6 Hz, 1H), 4.19 (s, 2H), 3.51 (q, J=6.0 Hz, 2H), 3.34 (t, J=5.6 Hz, 2H); LCMS (ESI) m/z 296.1 [C₁₈H₁₇NO₃+H]⁺.

### Example 3: Synthesis of Compound 7 Via Synthesis Route A

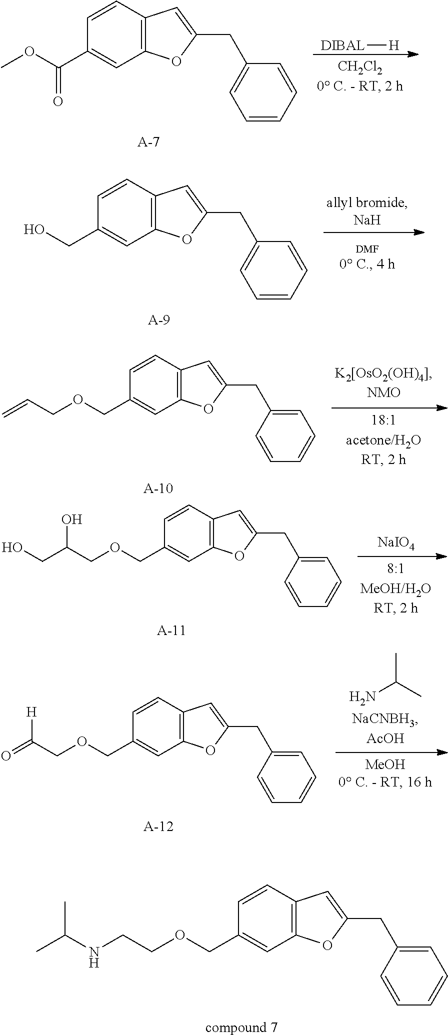

### Synthesis of (2-benzylbenzofuran-6-yl)methanol (A-9)

To a stirred solution of A-7 (450 mg, 1.68 mmol, 1.0 equiv.) in CH₂Cl₂ (5 mL) at 0° C., was added DIBAL-H (25% in toluene) (3 mL, 5.04 mmol, 3.0 equiv.). The resultant mixture was warmed to RT and stirred at RT for 2 h. Upon the completion of reaction by TLC, the reaction mixture was quenched with aq. NH₄Cl solution (30 mL) and extracted with CH₂Cl₂ (2×30 mL). The combined organic layers were dried over anhydrous Na₂SO₄, filtered, concentrated under reduced pressure, and purified by silica gel (60-120 mesh) column chromatography (gradient elution with 15-20% EtOAc/hexane) to afford A-9 as a yellow liquid (350 mg, yield: 87%). TLC system: EtOAc/hexane (20:80), R_{f} value=0.2; ¹H NMR (400 MHz, CDCl₃) δ ppm: 7.45-7.43 (m, 2H), 7.35-7.28 (m, 5H), 7.18 (dd, J=8.0 Hz, 1.2 Hz, 1H), 6.35 (s, 1H), 4.76 (s, 2H), 4.11 (s, 2H), 1.65 (br. s, 1H).

### Synthesis of 6-((allyloxy)methyl)-2-benzylbenzofuran (A-10)

To a stirred solution of A-9 (360 mg, 1.51 mmol, 1.0 equiv.) in DMF (3.6 mL) at 0° C., was added NaH (60% dispersion in mineral oil) (121 mg, 3.02 mmol, 2.0 equiv.) slowly. The reaction mixture was stirred at 0° C. for 20 min before allyl bromide (261 μL, 3.02 mmol, 2.0 equiv.) was added. The resultant mixture was stirred at 0° C. for 4 h. Upon the completion of reaction by TLC, the reaction mixture was quenched with ice-cold water and extracted with CH₂Cl₂ (3×50 mL). The combined organic layers were washed with brine solution, dried over Na₂SO₄, concentrated under reduced pressure, and purified by reverse phase column chromatography (Grace column, elution with 43% MeCN/0.1% FA in H₂O) to afford A-10 (185 mg, yield: 44%) as a colourless oily liquid. TLC system: EtOAc/hexane (20:80), R_{f} value=0.6; ¹H NMR (400 MHz, CDCl₃) δ ppm: 7.44-7.41 (m, 2H), 7.35-7.27 (m, 5H), 7.16 (dd, J=8.0 Hz, 1.6 Hz, 1H), 6.34 (s, 1H), 6.00-5.90 (m, 1H), 5.33-5.27 (m, 1H), 5.22-5.18 (m, 1H), 4.59 (s, 2H), 4.11 (s, 2H), 4.03-4.00 (m, 2H).

### Synthesis of 3-((2-benzylbenzofuran-6-yl)methoxy)propane-1,2-diol (A-11)

To a stirred solution of A-10 (180 mg, 0.65 mmol, 1.0 equiv.) in acetone (1.8 mL) and water (0.1 mL) at RT, was added K₂[OsO₂(OH)₄](47.9 mg, 0.13 mmol, 0.2 equiv.) and NMO (50% in water) (0.2 mL, 0.85 mmol, 1.3 equiv.). The resultant mixture was stirred at RT for 2 h. Upon the completion of reaction by TLC, water (20 mL) was added and the reaction was extracted with CH₂Cl₂ (3×50 mL). The combined organic layers were washed with brine solution, dried over Na₂SO₄, concentrated under reduced pressure, and purified by silica gel (100-200 mesh) column chromatography (elution with 35% EtOAc/hexane) to afford A-11 (125 mg, crude) as a brown liquid. TLC system: EtOAc/hexane (50:50), R_{f} value=0.3; ¹H NMR (400 MHz, CDCl₃) δ ppm: 7.43 (d, J=6.0 Hz, 1H), 7.38 (br. s, 1H), 7.35-7.27 (m, 4H→5H), 7.14 (dd, J=7.6 Hz, 1.2 Hz, 1H), 6.36 (s, 1H), 4.62 (s, 2H), 4.10 (s, 2H), 3.88 (br. s, 1H), 3.72-3.70 (m, 1H), 3.65-3.49 (m, 3H), 2.48 (br. s, 1H), 2.04 (br. s, 1H). Crude material was used in next step without further purification.

### Synthesis of 2-((2-benzylbenzofuran-6-yl)methoxy)acetaldehyde (A-12)

To a stirred solution of A-11 (crude from previous step) (120 mg, 0.38 mmol, 1.0 equiv.) in MeOH (2.4 mL) and H₂O (0.3 mL) at RT, was added NaIO₄ (130 mg, 0.61 mmol, 1.6 equiv.). The resultant mixture was stirred at RT for 2 h and upon the completion of reaction by TLC, the reaction mixture was diluted with H₂O (20 mL) and extracted with CH₂Cl₂ (3×50 mL). The combined organic layers were washed with brine solution, dried over Na₂SO₄, concentrated under reduced pressure, and purified by silica gel (100-200 mesh) column chromatography to afford A-12 (90 mg, yield: 49% over 2 steps) as a light green liquid. TLC system: EtOAc/hexane (50:50), R_{f} value=0.5; ¹H NMR (400 MHz, CDCl₃) δ ppm: 9.72 (s, 1H), 7.45 (d, J=8.0 Hz, 1H), 7.42 (s, 1H), 7.34-7.27 (m, 5H), 7.17 (dd, J=8.0 Hz, 1.2 Hz, 1H), 6.37 (s, 1H), 4.70 (s, 2H), 4.11 (s, 2H), 3.49 (s, 2H).

### Synthesis of N-(2-((2-benzylbenzofuran-6-yl)methoxy)ethyl)propan-2-amine (Compound 7)

To a stirred solution of A-12 (90 mg, 0.32 mmol, 1.0 equiv.) in MeOH (1 mL) at RT, was added isopropylamine (68.1 μL, 0.80 mmol, 2.5 equiv.) and AcOH (4 μL, 0.06 mmol, 0.2 equiv.). The reaction mixture was cooled to 0° C. and NaCNBH₃ (50.3 mg, 0.80 mmol, 2.5 equiv.) was added. The resultant mixture was warmed to RT and stirred at RT for 16 h. Upon the completion of reaction by TLC, the reaction mixture was quenched with ice-cold water and extracted with CH₂Cl₂. The organic layer was dried over Na₂SO₄, concentrated under reduced pressure, and the crude obtained was purified by reverse phase column chromatography (Grace column, elution with 35% MeCN/0.1% FA in H₂O) to afford compound 7 (30 mg, yield: 29%) as an off-white semi solid. TLC system: MeOH/CH₂Cl₂ (10:90), R_{f} value=0.2; ¹H NMR (400 MHz, DMSO-d₆) δ ppm: 7.52 (d, J=8.0 Hz, 1H), 7.47 (s, 1H), 7.34-7.30 (m, 4H), 7.27-7.25 (m, 1H), 7.17 (dd, J=7.6 Hz, 1.2 Hz, 1H), 6.61 (s, 1H), 4.57 (s, 2H), 4.14 (s, 2H), 3.54 (t, J=5.6 Hz, 2H), 2.98-2.92 (m, 1H), 2.87 (t, J=5.6 Hz, 2H), 1.06 (d, J=6.4 Hz, 6H); LCMS (ESI) m/z 324.3 [C₂₁H₂₅NO₂+H]⁺.

### Example 4: Synthesis of Compound 36 Via Synthesis Route A

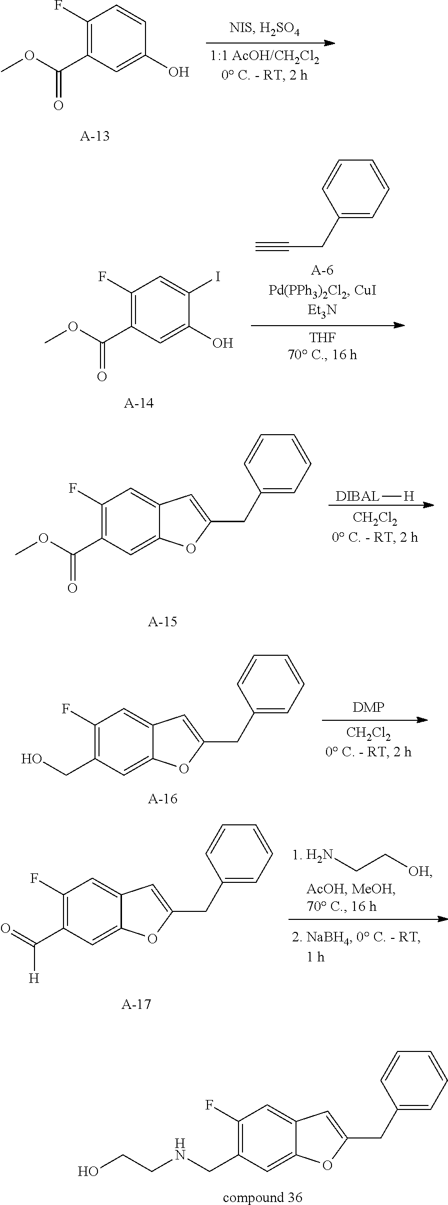

### Synthesis of methyl 2-fluoro-5-hydroxy-4-iodobenzoate (A-14)

To a stirred solution of A-13 (1.75 g, 10.3 mmol, 1.0 equiv.) in 1:1 AcOH/CH₂Cl₂ (35 mL, 20 vol) at 0° C., was added H₂SO₄ (0.7 mL, 0.4 vol) and NIS (2.79 g, 12.4 mmol, 1.2 equiv.). The reaction mixture was stirred at RT for 2 h and upon the completion of reaction by TLC, the reaction mixture was quenched with ice-cold water (100 mL) and extracted with CH₂Cl₂ (2×100 mL). The combined organic layers were dried over anhydrous Na₂SO₄, filtered, and concentrated under reduced pressure. The crude obtained was purified by reverse phase column chromatography (Grace column, elution with 28% MeCN/0.1% FA in H₂O) to afford A-14 (852 mg, yield: 28%) as a brown oil. TLC system: EtOAc/hexane (20:80), R_{f} value=0.5; ¹H NMR (400 MHz, DMSO-d₆) δ ppm: 10.65 (s, 1H), 7.72 (d, J=10 Hz, 1H), 7.31 (d, J=6.4 Hz, 1H), 3.83 (s, 3H); LCMS (ESI) m/z 294.8 [C₈H₆FIO₃−H]⁻.

### Synthesis of methyl 2-benzyl-5-fluorobenzofuran-6-carboxylate (A-15)

Compound A-15 was obtained following the Pd-catalyzed benzofuran ring formation procedure similar to that for the synthesis of compound A-3, utilizing prop-2-yn-1-ylbenzene A-6 (1.2 equiv.) and reaction conditions of 70° C. for 16 h. Compound A-15 was obtained in 65% yield as a brown oil following silica gel (60-120 mesh) column chromatography (elution with 5% EtOAc/hexane). TLC system: EtOAc/hexane (5:95), R_{f} value=0.7; ¹H NMR (400 MHz, CDCl₃) δ ppm: 7.98 (d, J=5.6 Hz, 1H), 7.37-7.30 (m, 5H), 7.17 (d, J=10.8 Hz, 1H), 6.36 (s, 1H), 4.12 (s, 2H), 3.94 (s, 3H); LCMS (ESI) m/z 285.1 [C₁₇H₁₃FO₃+H]⁺.

### Synthesis of (2-benzyl-5-fluorobenzofuran-6-yl)methanol (A-16)

Compound A-16 was obtained following the ester reduction procedure similar to that for the synthesis of compound A-4, utilizing DIBAL-H (2.0 equiv.) and reaction conditions of 0° C. -RT for 2 h. Compound A-16 was obtained as a crude and was used directly in the next step without further purification. TLC system: EtOAc/hexane (30:70), R_{f} value=0.7; ¹H NMR (400 MHz, CDCl₃) δ ppm: 7.43 (d, J=5.6 Hz, 1H), 7.35-7.28 (m, 5H), 7.12 (d, J=9.6 Hz, 1H) 6.33 (s, 1H), 4.80 (s, 2H), 4.09 (s, 2H), 1.80 (br. s, 1H); LCMS (ESI)ₘ z 239.1 [C₁₆H₁₃FO₂
OH]⁺.

### Synthesis of 2-benzyl-5-fluorobenzofuran-6-carbaldehyde (A-17)

Compound A-17 was obtained following the alcohol oxidation procedure similar to that for the synthesis of compound A-5, utilizing DMP (2.0 equiv.) and reaction conditions of 0° C.
RT for 2 h. Compound A-17 was obtained in 89% yield over 2 steps as a brown oil following silica gel (60-120 mesh) column chromatography (elution with 10% EtOAc/hexane). TLC system: EtOAc/hexane (20:80), R_{f} value=0.4; ¹H NMR (400 MHz, CDCl₃) δ ppm: 10.36 (s, 1H), 7.88 (d, J=5.2 Hz, 1H), 7.36-7.29 (m, 5H), 7.19 (d, J=10.0 Hz, 1H) 6.40 (s, 1H), 4.13 (s, 2H); LCMS (ESI) m/z 255.1 [C₁₆H₁₁FO₂+H]⁺.

### Synthesis of 2-(((2-benzyl-5-fluorobenzofuran-6-yl)methyl)amino)ethan-1-ol as Formic Acid Salt (Compound 36)

Compound 36 was obtained following the reductive amination procedure similar to that for the synthesis of compound 2, utilizing 2-aminoethan-1-ol (2.0 equiv.), AcOH (0.1 equiv.), NaBH₄ (2.0 equiv.) and reaction conditions of 70° C. for 16 h for imine formation, and 0° C. to RT for 1 h for imine reduction. Compound 36 (formic acid salt) was obtained in 28% yield as a light brown semi-solid following reverse phase column chromatography (Grace column, elution with 17% MeCN/0.1% FA in H₂O). TLC system: MeOH/CH₂Cl₂ (10:90), R_{f} value=0.2; ¹H NMR (400 MHz, DMSO-d₆) δ ppm: 8.22 (s, 1H, HCOOH), 7.56 (d, J=6.0 Hz, 1H), 7.36-7.30 (m, 5H), 7.27-7.25 (m, 1H), 6.58 (s, 1H), 4.14 (s, 2H), 3.84 (s, 2H), 3.48 (t, J=5.6 Hz, 2H), 2.62 (t, J=5.6 Hz, 2H); LCMS (ESI) m/z 300.1 [C₁₈H₁₈FNO₂+H]⁺.

### Example 5: Synthesis of Compound 9 Via Synthesis Route B

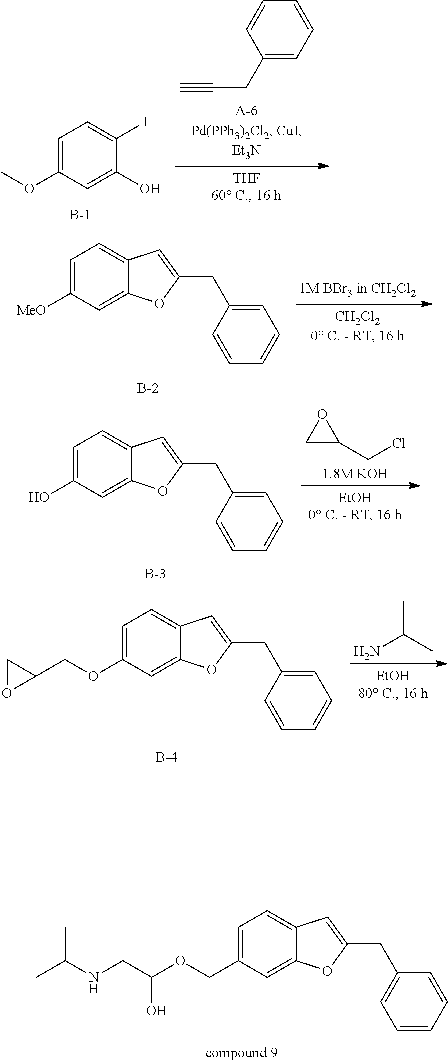

### Synthesis of 2-benzyl-6-methoxybenzofuran (B-2)

Compound B-2 was obtained following the Pd-catalyzed benzofuran ring formation procedure similar to that for the synthesis of compound A-3, utilizing prop-2-yn-1-ylbenzene A-6 (1.2 equiv.) and reaction conditions of 60° C. for 16 h. Compound B-2 was obtained semi-pure as a brown gum following silica gel (60-120 mesh) column chromatography (elution with 50% EtOAc/hexane). TLC system: EtOAc/hexane (20:80), R_{f} value=0.3; ¹H NMR (400 MHz, CDCl₃) δ ppm: 7.35-7.29 (m, 6H), 6.96 (s, 1H), 6.81 (dd, J=8.8 Hz, 2.4 Hz, 1H), 6.30 (s, 1H), 4.07 (s, 2H), 3.82 (s, 3H).

### Synthesis of 2-benzylbenzofuran-6-ol (B-3)

To a stirred solution of B-2 (500 mg, 2.10 mmol, 1.0 equiv.) in CH₂Cl₂ (5 mL) at 0° C., was added 1 M BBr₃ in CH₂Cl₂ (4.20 mL, 4.20 mmol, 2.0 equiv.). The resultant mixture was warmed to RT and stirred at RT for 16 h. Upon the completion of reaction by TLC, the reaction was quenched with aq. NaHCO₃ solution (30 mL) and extracted with CH₂Cl₂ (2×50 mL). The combined organic layers were dried over anhydrous Na₂SO₄, filtered, concentrated under reduced pressure, and purified by silica gel (60-120 mesh) column chromatography (elution with 20% EtOAc/hexane) to afford B-3 as pale-yellow gum (380 mg, yield: 42% over 2 steps). TLC system: EtOAc/hexane (50:50), R_{f} value=0.3; ¹H NMR (400 MHz, CDCl₃) δ ppm: 7.39-7.28 (m, 6H), 6.94 (d, J=2.4 Hz, 1H), 6.75 (dd, J=8.4 Hz, 2.4 Hz, 1H), 6.32 (s, 1H), 4.89 (br. s, 1H), 4.10 (s, 2H).

### Synthesis of 2-benzyl-6-(oxiran-2-ylmethoxy)benzofuran (B-4)

To a stirred solution of B-3 (370 mg, 1.65 mmol, 1.0 equiv.) in EtOH (4 mL) at 0° C., was added 1.8 M aq. KOH solution (3.8 mL). The reaction mixture was stirred at 0° C. for 15 min before 2-(chloromethyl)oxirane (0.26 mL, 3.30 mmol, 2.0 equiv.) was added. The resultant mixture was warmed to RT and stirred at RT for 16 h. Upon the completion of reaction by TLC, the volatiles were removed, the residue was diluted with H₂O (30 mL) and extracted with CH₂Cl₂ (2×50 mL). The combined organic layers were dried over anhydrous Na₂SO₄, filtered, and concentrated. The crude obtained was purified by silica gel (100-200 mesh) column chromatography (elution with 10% EtOAc/hexane) to afford B-4 as a brown gummy mass (160 mg, yield: 35%). TLC system: EtOAc/hexane (50:50), R_{f} value=0.7; ¹H NMR (400 MHz, CDCl₃) δ ppm: 7.34-7.29 (m, 5H), 7.25-7.23 (m, 1H), 6.97 (d, J=2.4 Hz, 1H), 6.84 (dd, J=8.4 Hz, 2.4 Hz, 1H), 6.29 (s, 1H), 4.24-4.21 (m, 1H), 4.07 (s, 2H), 3.99-3.95 (m, 1H), 3.39-3.35 (m, 1H), 2.92-2.90 (m, 1H), 2.78-2.76 (m, 1H); LCMS (ESI) m/z 281.1 [C₁₈H₁₆O₃+H]⁺.

### Synthesis of 1-((2-benzylbenzofuran-6-yl)oxy)-3-(isopropylamino)propan-2-ol as Formic Acid Salt (Compound 9)

To a stirred solution of B-4 (160 mg, 0.57 mmol, 1.0 equiv.) in EtOH (3.2 mL) at RT, was added isopropylamine (0.24 mL, 2.85 mmol, 5.0 equiv.). The resultant mixture was stirred at 80° C. for 16 h. Upon the completion of reaction by TLC, the volatiles were removed, and the crude obtained was purified by reverse phase column chromatography (Grace column, elution with 17% MeCN/0.01% FA in H₂O) to afford compound 9 (formic acid salt) as a light brown gum (51 mg, yield: 23%). TLC system: MeOH/CH₂Cl₂ (05:95), R_{f} value=0.1; ¹H NMR (400 MHz, DMSO-d₆) δ ppm: 8.34 (br. s, 1H; HCOOH), 7.41 (d, J=8.4 Hz, 1H), 7.35-7.29 (m, 4H), 7.26-7.23 (m, 1H), 7.11 (s, 1H), 6.83 (dd, J=8.4 Hz, 2.4 Hz, 1H), 6.52 (s, 1H), 4.10 (s, 2H), 4.05-4.01 (m, 1H), 3.96-3.93 (m, 2H), 3.06-3.01 (m, 1H), 2.94-2.90 (m, 1H), 2.80-2.75 (m, 1H), 1.13-1.11 (m, 6H); LCMS (ESI) m/z 340.3 [C₂₁H₂₅NO₃+H]⁺.

### Example 6: Synthesis of Compound 11 Via Synthesis Route C

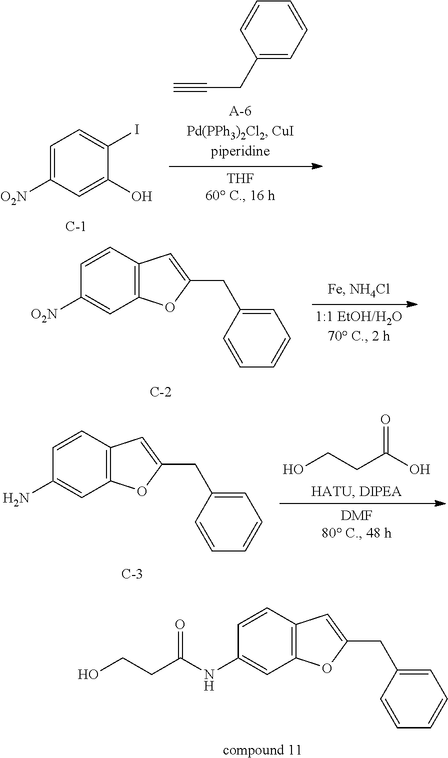

### Synthesis of 2-benzyl-6-nitrobenzofuran (C-2)

Compound C-2 was obtained following the Pd-catalyzed benzofuran ring formation procedure similar to that for the synthesis of compound A-3, utilizing prop-2-yn-1-ylbenzene A-6 (1.0 equiv.), piperidine (1.0 equiv.) and reaction conditions of 60° C. for 16 h. Compound C-2 was obtained in 57% yield as a brown gum following silica gel (100-200 mesh) column chromatography (elution with 5% EtOAc/hexane). TLC system: EtOAc/hexane (30:70), R_{f} value=0.7; ¹H NMR (400 MHz, CDCl₃) δ ppm: 8.30 (s, 1H), 8.12 (dd, J=8.8 Hz, 2.0 Hz, 1H), 7.52 (d, J=8.4 Hz, 1H), 7.38-7.27 (m, 5H), 6.47 (s, 1H), 4.16 (s, 2H).

### Synthesis of 2-benzylbenzofuran-6-amine (C-3)

To a stirred solution of C-2 (470 mg, 1.86 mmol, 1.0 equiv.) in 1:1 EtOH/H₂O (4.60 mL) at RT, was added Fe powder (312 mg, 5.58 mmol, 3.0 equiv.) and NH₄Cl (298 mg, 5.58 mmol, 3.0 equiv.). The resultant mixture was stirred at 70° C. for 2 h. Upon the completion of reaction by TLC, the reaction mixture was filtered through a Celite bed, washing with EtOAc. The filtrate was diluted with H₂O and extracted with EtOAc (2×50 mL). The combined organic layers were dried over anhydrous Na₂SO₄, concentrated under reduced pressure, and triturated with n-pentane to afford C-3 as a brown solid (230 mg, yield: 55%). TLC system: EtOAc/hexane (30:70), R_{f} value=0.1; ¹H NMR (400 MHz, CDCl₃) δ ppm: 7.33-7.29 (m, 5H), 7.21 (d, J=8.0 Hz, 1H), 6.74 (s, 1H), 6.57 (dd, J=8.0 Hz, 1.6 Hz, 1H), 6.23 (s, 1H), 4.05 (s, 2H); LCMS (ESI) m/z 224.2 [C₁₅H₁₃NO+H]⁺.

### Synthesis of N-(2-benzylbenzofuran-6-yl)-3-hydroxypropanamide (Compound 11)

To a stirred solution of C-3 (185 mg, 0.83 mmol, 1.0 equiv.) in DMF (1.85 mL) at 0° C., was added HATU (475 mg, 1.25 mmol, 1.5 equiv.) and DIPEA (0.29 mL, 1.66 mmol, 2.0 equiv.). The reaction mixture was warmed to RT and stirred at RT for 15 min before 3-hydroxypropanoic acid (30% in H₂O) (0.41 mL, 4.98 mmol, 6.0 equiv.) was added. The resultant mixture was stirred at 80° C. for 48 h. Upon the completion of reaction by TLC, the reaction was quenched with ice-cold water (15 mL) and extracted with EtOAc (2×20 mL). The combined organic layers were dried over anhydrous Na₂SO₄ and concentrated under reduced pressure. The crude obtained was purified by reverse phase column chromatography (Grace column, gradient elution with 30-40% MeCN/0.01% FA in H₂O) to afford compound 11 as a white solid (28 mg, yield: 11%). TLC system: EtOAc/hexane (50:50), R_{f} value=0.2; melting point range: 128-131° C.; ¹H NMR (400 MHz, DMSO-d₆) δ ppm: 9.98 (s, 1H), 7.96 (s, 1H), 7.42 (d, J=8.4 Hz, 1H), 7.35-7.30 (m, 4H), 7.27-7.23 (m, 2H), 6.51 (s, 1H), 4.67 (t, J=5.2 Hz, 1H), 4.11 (s, 2H), 3.71 (q, J=5.6 Hz, 2H), 2.46 (t, J=6.4 Hz, 2H); LCMS (ESI) m/z 296.1 [C₁₈H₁₇NO₃+H]⁺.

### Example 7: Synthesis of Compound 13 Via Synthesis Route D

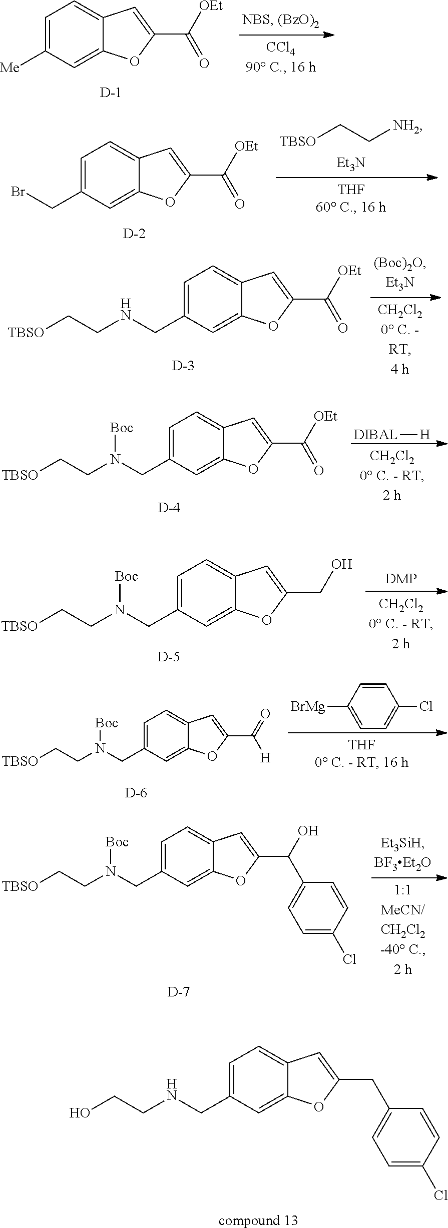

### Synthesis of ethyl 6-(bromomethyl)benzofuran-2-carboxylate (D-2)

To a solution of carboxylate D-1 (6.70 g, 32.8 mmol, 1.0 equiv.) in CCl₄ (140 mL) at RT, was added NBS (7.58 g, 42.6 mmol, 1.3 equiv.) and benzoic peroxyanhydride (795 mg, 3.28 mmol, 0.1 equiv.). The resultant mixture was stirred at 90° C. for 16 h and upon the completion of reaction by TLC, the mixture was diluted with H₂O and extracted with CH₂Cl₂ (2×100 mL). The combined organic layers were dried over Na₂SO₄ and evaporated to afford D-2 as a brown solid (9.20 g, crude). TLC system: EtOAc/hexane (20:80), R_{f} value=0.4; LCMS (ESI) m/z 283.0 [Cl₂H₁₁BrO₃+H]⁺. The crude material was used in the next step without purification.

### Synthesis of ethyl 6-(((2-((tert-butyldimethylsilyl)oxy)ethyl)amino)methyl)benzofuran-2-carboxylate (D-3)

To a stirred solution of D-2 (9.20 g, 32.5 mmol, 1.0 equiv.) in THE (100 mL) at RT, was added 2-((tert-butyldimethylsilyl)oxy)ethan-1-amine (7.40 mL, 35.8 mmol, 1.1 equiv.) and NEt₃ (6.80 mL, 48.8 mmol, 1.5 equiv.). The resultant mixture was stirred at 60° C. for 16 h and upon the completion of reaction by TLC, the mixture was diluted with H₂O and extracted with CH₂Cl₂ (2×100 mL). The combined organic layers were washed with brine (100 mL), dried over Na₂SO₄, filtered and concentrated. The crude compound was purified by silica gel (60-120 mesh) column chromatography (elution with 20% EtOAc/hexane) to afford D-3 as a brown colour gum (4.70 g, yield: 38%). TLC system: EtOAc/hexane (20:80), R_{f} value=0.3; ¹H NMR (400 MHz, CDCl₃) δ ppm: 7.62 (d, J=8.0 Hz, 1H), 7.57 (s, 1H), 7.50 (d, J=0.8 Hz, 1H), 7.30 (dd, J=8.0 Hz, 1.2 Hz, 1H), 4.46 (q, J=7.2 Hz, 2H), 3.96 (s, 2H), 3.75-3.68 (m, 2H), 2.76 (t, J=5.2 Hz, 2H), 1.43 (t, J=7.2 Hz, 3H), 0.89 (s, 9H), 0.06 (s, 6H); LCMS (ESI) m/z 378.2 [C₂₀H₃₁NO₄Si+H]⁺.

### Synthesis of ethyl 6-(((tert-butoxycarbonyl)(2-((tert-butyldimethylsilyl)oxy)ethyl)amino)methyl)benzofuran-2-carboxylate (D-4)

To a solution of D-3 (4.40 g, 11.7 mmol, 1.0 equiv.) in CH₂Cl₂ (50 mL) at 0° C., was added NEt₃ (2.45 mL, 17.6 mmol, 1.5 equiv.) and (Boc)₂O (3.49 mL, 15.2 mmol, 1.3 equiv.). The resultant mixture was warmed to RT and stirred at RT for 4 h. Upon the completion of reaction by TLC, the reaction mixture was diluted with H₂O and extracted with CH₂Cl₂ (2×70 mL). The combined organic layers were washed with brine, dried over Na₂SO₄, filtered, and concentrated. The crude compound was purified by silica gel (100-200 mesh) column chromatography (elution with 10% EtOAc/hexane) to afford D-4 as a pale-yellow oil (4.80 g, yield: 86%). TLC system: EtOAc/hexane (20:80), R_{f} value=0.8; ¹H NMR (400 MHz, CDCl₃) δ ppm: 7.61 (d, J=8.4 Hz, 1H), 7.50 (s, 1H), 7.46 (br. s, 1H), 7.21-7.17 (m, 1H), 4.66 (d, J=6.8 Hz, 2H), 4.44 (q, J=7.2 Hz, 2H), 3.78-3.69 (m, 2H), 3.36-3.26 (m, 2H), 1.52-1.50 (d, 9H), 1.43 (t, J=7.2 Hz, 3H), 0.89 (s, 9H), 0.04 (s, 6H); LCMS (ESI) m/z 478.2 [C₂₅H₃₉NO₆Si+H]⁺.

### Synthesis of tert-butyl (2-((tert-butyldimethylsilyl)oxy)ethyl)((2-(hydroxymethyl)benzofuran-6-yl)methyl)carbamate (D-5)

To a stirred solution of D-4 (4.50 g, 9.42 mmol, 1.0 equiv.) in CH₂Cl₂ (50 mL) at 0° C., was added DIBAL-H (25% in toluene) (15.9 mL, 23.6 mmol, 2.5 equiv.) drop-wise. The resultant mixture was warmed to RT and stirred at RT for 2 h. Upon the completion of reaction by TLC, the reaction mixture was quenched with sat. NH₄Cl solution, filtered through a Celite bed and extracted with CH₂Cl₂ (3×50 mL). The combined organic layers were dried over anhydrous Na₂SO₄, filtered, and concentrated. The crude compound was purified by silica gel (60-120 mesh) column chromatography (elution with 15% EtOAc/hexane) to afford D-5 as a pale-yellow oil (2.90 g, yield: 66%). TLC system: EtOAc/hexane (30:70), R_{f} value=0.3; ¹H NMR (400 MHz, CDCl₃) δ ppm: 7.47 (d, J=8.0 Hz, 1H), 7.33 (d, J=8.0 Hz, 1H), 7.13-7.08 (m, 1H), 6.63 (s, 1H), 4.76 (s, 2H), 4.62 (s, 2H), 3.76-3.67 (m, 2H), 3.33-3.24 (m, 2H), 1.99 (br. s, 1H), 1.50-1.45 (d, 9H), 0.89 (s, 9H), 0.04 s, 6H); LCMS (ESI) m/z 436.2 [C₂₃H₃₇NO₅Si+H]⁺.

### Synthesis of tert-butyl (2-((tert-butyldimethylsilyl)oxy)ethyl)((2-formylbenzofuran-6-yl)methyl)carbamate (D-6)

To a stirred solution of D-5 (1.50 g, 3.44 mmol, 1.0 equiv.) in CH₂Cl₂ (30 mL) at 0° C., was added Dess-Martin periodinane (2.92 g, 6.88 mmol, 2.0 equiv.). The resultant mixture was warmed to RT and stirred at RT for 2 h. Upon the completion of reaction by TLC, the reaction mixture was quenched with saturated aq. NaHCO₃ solution (50 mL) and extracted with CH₂Cl₂ (3×50 mL). The combined organic layers were dried over anhydrous Na₂SO₄, filtered, and concentrated under reduced pressure to afford the crude product. The crude product was purified by column chromatography using silica gel (100-200 mesh) column (elution with 20% EtOAc/hexane) to afford D-6 as a colourless liquid (1.10 g, yield: 74%). TLC system: EtOAc/hexane (20:80), R_{f} value=0.6; ¹H NMR (400 MHz, CDCl₃) δ ppm: 9.88 (s, 1H), 7.71 (d, J=8.4 Hz, 1H), 7.56 (s, 1H), 7.49 (s, 1H), 7.28-7.26 (m, 1H), 4.71-4.69 (d, J=6.8 Hz, 2H), 3.81-3.73 (m, 2H), 3.39-3.30 (m, 2H), 1.53-1.44 (d, 9H), 0.91 (s, 9H), 0.07 (s, 6H); LCMS (ESI) m/z 434.3 [C₂₃H₃₅NO₅Si+H]⁺.

### Synthesis of tert-butyl (2-((tert-butyldimethylsilyl)oxy)ethyl)((2-((4-chlorophenyl)(hydroxy)methyl)benzofuran-6-yl)methyl)carbamate (D-7)

To a stirred solution of D-6 (500 mg, 1.15 mmol, 1.0 equiv.) in THE (5 mL) at 0° C., was added (4-chlorophenyl)magnesium bromide (1 M in THF, 3.45 mL, 3.45 mmol, 3 equiv.). The resultant mixture was warmed to RT and stirred at RT for 16 h. Upon the completion of reaction by TLC, the reaction mixture was diluted with H₂O and extracted with CH₂Cl₂ (2×50 mL). The combined organic layers were washed with brine (50 mL), dried with Na₂SO₄, filtered, and concentrated. The crude obtained was purified by silica gel (100-200 mesh) column chromatography (elution with 10% EtOAc/hexane) to afford D-7 as a gummy liquid (0.35 g, yield: 56%). TLC system: EtOAc/hexane (30:70), R_{f} value=0.2; ¹H NMR (400 MHz, CDCl₃) δ ppm: 7.45-7.42 (m, 3H), 7.36 (d, J=8.8 Hz, 2H), 7.32-7.30 (m, 1H), 7.12-7.07 (m, 1H), 6.50 (s, 1H), 5.92 (d, J=4.0 Hz, 1H), 4.61 (s, 2H), 3.75-3.67 (m, 2H), 3.31-3.23 (m, 2H), 2.57 (d, J=4.4 Hz, 1H), 1.49-1.44 (d, 9H), 0.89 (s, 9H), 0.03 (s, 6H); LCMS (ESI) m/z 546.2 [C₂₉H₄₀ClNO₅Si+H]⁺.

### Synthesis of 2-(((2-(4-chlorobenzyl)benzofuran-6-yl)methyl)amino)ethan-1-ol as Formic Acid Salt (Compound 13)

To a stirred solution of D-7 (350 mg, 0.64 mmol, 1.0 equiv.) in 1:1 MeCN/CH₂Cl₂ (7.0 mL) at −40° C., was added BF₃·Et₂O (0.13 mL, 1.15 mmol, 1.8 equiv.), followed by Et₃SiH (0.3 mL, 1.92 mmol, 3 equiv.). The resultant mixture was allowed to stir at −40° C. for 2 h. Upon the completion of reaction by TLC, the reaction mixture was concentrated under reduced pressure and purified by reverse phase column chromatography (Grace column, elution with 17% MeCN/0.1% FA in H₂O) to afford the compound as a BF₃ salt. This material was treated with isopropyl amine (5 equiv.) in THF (12 mL) at 40° C. and upon the absence of fluorine signal by 19F NMR, the mixture was concentrated and passed through a reverse phase column to afford compound 13 (formic acid salt) as an off-white solid (24 mg, yield: 10%). TLC system: MeOH/CH₂Cl₂ (10:90), R_{f} value=0.1; melting point range: 58-62° C.; ¹H NMR (400 MHz, DMSO-d₆) δ ppm: 8.20 (s, 1H; HCOOH), 7.50-7.48 (m, 2H), 7.39 (d, J=8.4 Hz, 2H), 7.33 (d, J=8.4 Hz, 2H), 7.20 (d, J=8.0 Hz, 1H), 6.60 (s, 1H), 4.15 (s, 2H), 3.89 (s, 2H), 3.49 (t, J=5.6 Hz, 2H), 2.63 (t, J=5.6 Hz, 2H); LCMS (ESI) m/z 316.2 [C₁₈H₁₈ClNO₂+H]⁺.

### Example 8: Synthesis of Compound 33 Via Synthesis Route D

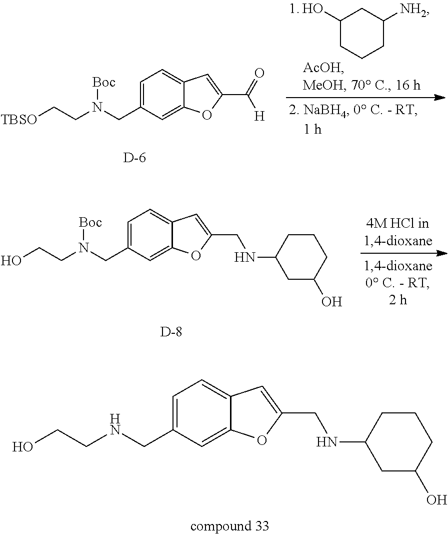

### Synthesis of tert-butyl ((2-(((3-hydroxycyclohexyl)amino)methyl)benzofuran-6-yl)methyl)(2-hydroxyethyl)carbamate (D-8)

Compound D-8 was obtained following the reductive amination procedure similar to that for the synthesis of compound 2, utilizing 3-aminocyclohexan-1-ol (1.5 equiv.) with NaBH₄ (2.0 equiv.) and reaction conditions of 70° C. for 16 h for imine formation, and 0° C. to RT for 1 h for imine reduction. Compound D-8 was obtained in 45% yield as a brown liquid following reverse phase column chromatography (Grace column, gradient elution with 20-30% MeCN/0.1% FA in H₂O). TLC system: MeOH/CH₂Cl₂ (10:90), R_{f} value=0.2; LCMS (ESI) m/z 419.1 [C₂₃H₃₄N₂O₅+H]⁺.

### Synthesis of 3-(((6-(((2-hydroxyethyl)amino)methyl)benzofuran-2-yl)methyl)amino)cyclohexan-1-ol as bis-formic acid salt (Compound 33)

To a stirred solution of D-8 (130 mg, 0.31 mmol, 1.0 equiv.) in 1,4-Dioxane (0.33 mL) at 0° C., was added 4 M HCl in 1,4-Dioxane (0.33 mL). The resultant mixture was warmed to RT and and stirred at RT for 2 h. Upon the completion of reaction by TLC, the reaction mixture was concentrated under reduced pressure and purified by reverse phase column chromatography (Grace column, gradient elution with 0-6% MeCN/0.1% FA in H₂O) to afford compound 33 (bis-formic acid salt) as an off-white gummy solid (52 mg, yield: 18% for 2 steps). TLC system: MeOH/CH₂Cl₂ (10:90), R_{f} value=0.1; ¹H NMR (400 MHz, DMSO-d₆) δ ppm: 8.32 (br. s, 2H; HCOOH), 7.50-7.48 (m, 2H), 7.19 (d, J=8.0 Hz, 1H), 6.67 (s, 1H), 3.87-3.84 (m, 4H), 3.50 (t, J=5.6 Hz, 2H), 3.36-3.31 (m, 1H), 2.86-2.84 (m, 1H), 2.67-2.63 (m, 2H), 2.44-2.40 (m, 1H), 1.82-1.73 (m, 1H), 1.65-1.62 (m, 1H), 1.49-1.37 (m, 2H), 1.18-0.87 (m, 3H); LCMS (ESI) m/z 319.2 [C₁₈H₂₆N₂O₃+H]⁺.

### Example 9: Synthesis of Compound 49 Via Synthesis Route D

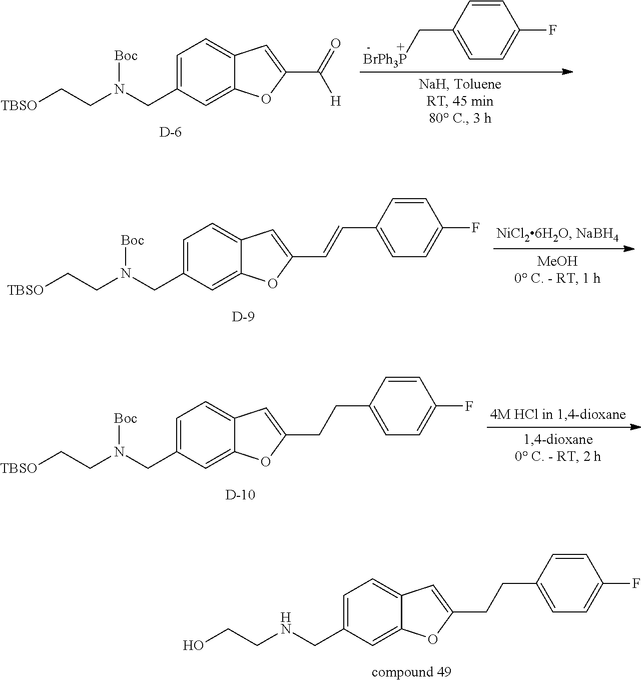

### Synthesis of tert-butyl (E/Z)-(2-((tert-butyldimethylsilyl)oxy)ethyl)((2-(4-fluorostyryl)benzofuran-6-yl)methyl)carbamate (D-9)

To a stirred solution of (4-fluorobenzyl)triphenylphosphonium bromide (1.17 g, 2.60 mmol, 2.5 equiv.) in dry toluene (4.5 mL) at 0° C., was added NaH (60% in mineral oil) (104 mg, 2.60 mmol, 2.5 equiv.). The reaction mixture was warmed to RT and stirred at RT for 45 min before the reaction mixture was cooled back to 0° C., and D-6 (450 mg, 1.04 mmol, 1.0 equiv.) was added. The resultant mixture was heated to 80° C. and stirred at 80° C. for 3 h. Upon the completion of reaction by TLC, the reaction mixture was diluted with H₂O and extracted with EtOAc (2×50 mL). The combined organic layers were washed with brine, dried over Na₂SO₄, filtered, and concentrated. The crude obtained was purified by silica gel (60-120 mesh) column chromatography (elution with 10% EtOAc/hexane) to afford D-9 (mixture of E and Z isomers in 1:1 ratio) as a colourless liquid (380 mg, yield: 69%). TLC system: EtOAc/hexane (10:90), R_{f} value=0.3; LCMS (ESI) m/z 526.4 [C₃₀H₄₀FNO₄Si+H]⁺.

### Synthesis of tert-butyl (2-((tert-butyldimethylsilyl)oxy)ethyl)((2-(4-fluorophenethyl)benzofuran-6-yl)methyl)carbamate (D-10)

To a stirred solution of D-9 (mixture of E and Z isomers) (300 mg, 0.57 mmol, 1.0 equiv.) in MeOH (3 mL) at 0° C., was added NiCl₂·6H₂O (13.6 mg, 0.06 mmol, 0.1 equiv.) and NaBH₄ (64.7 mg, 1.71 mmol, 3.0 equiv.). The resultant mixture was warmed to RT and stirred at RT for 1 h. Upon the completion of reaction by TLC, the reaction mixture was quenched with ice-cold water (30 mL) and extracted with EtOAc (2×40 mL). The combined organic layers were dried over anhydrous Na₂SO₄, filtered, and concentrated under reduced pressure to afford D-10 as a colourless liquid (280 mg, crude). TLC system: EtOAc/hexane (10:90), R_{f} value=0.3; LCMS (ESI) m/z 528.2 [C₃₀H₄₂FNO₄Si+H]⁺. The crude material was taken forward to the next step without further purification.

### Synthesis of 2-(((2-(4-fluorophenethyl)benzofuran-6-yl)methyl)amino)ethan-1-ol as Formic Acid Salt (Compound 49)

To a stirred solution of D-10 (270 mg, 0.51 mmol, 1.0 equiv.) in 1,4-Dioxane (3.0 mL) at 0° C., was added 4 M HCl in 1,4-Dioxane (1.4 mL). The resultant mixture was warmed to RT and stirred at RT for 2 h. Upon the completion of reaction by TLC, the volatiles were removed and the crude obtained was purified by reverse phase column chromatography (Grace column, gradient elution with 20-25% MeCN/0.1% FA in H₂O) to afford compound 49 (formic acid salt) as an off-white solid (60 mg, yield: 33%). TLC system: MeOH/CH₂Cl₂ (10:90), R_{f} value=0.2; melting point range: 49-51° C.; ¹H NMR (400 MHz, DMSO-d₆) δ ppm: 8.24 (s, 1H; HCOOH), 7.51 (s, 1H), 7.46 (d, J=8.0 Hz, 1H), 7.31-7.27 (m, 2H), 7.19 (dd, J=8.0 Hz, 0.8 Hz, 1H), 7.11-7.07 (m, 2H), 6.54 (s, 1H), 3.91 (s, 2H), 3.52 (t, J=5.6 Hz, 2H), 3.09-3.06 (m, 2H), 3.00-2.98 (m, 2H), 2.67 (t, J=5.6 Hz, 2H); LCMS (ESI) m/z 314.3 [C₁₉H₂₀FNO₂+H]⁺.

### Example 10: Synthesis of Compound 34 Via Synthesis Route D

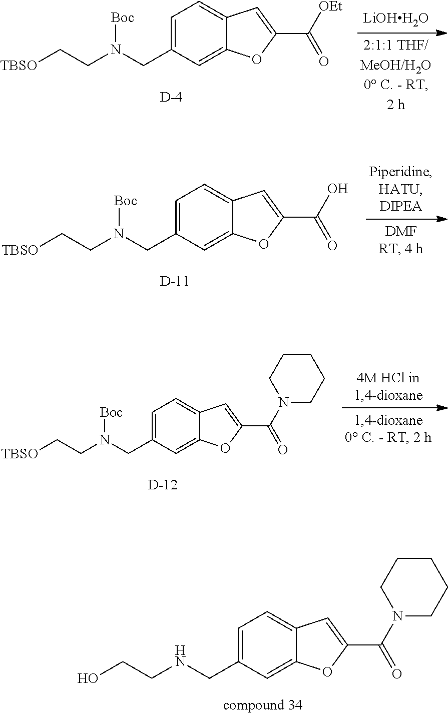

### Synthesis of 6-(((tert-butoxycarbonyl)(2-((tert-butyldimethylsilyl)oxy)ethyl)amino)methyl)benzofuran-2-carboxylic acid (D-11)

To a stirred solution of D-4 (300 mg, 0.63 mmol, 1.0 equiv.) in a mixture of 2:1:1 THF/MeOH/H₂O (2 mL) at 0° C., was added LiOH·H₂O (31.9 mg, 0.76 mmol, 1.2 equiv.). The resultant mixture was allowed to warm to RT and stirred at RT for 2 h. Upon the completion of reaction by TLC, the reaction mixture was neutralized with aq. citric acid solution and extracted with CH₂Cl₂ (3×30 mL). The combined organic layers were dried over Na₂SO₄ and concentrated under reduced pressure to afford D-11 as a pale-yellow solid (267 mg) in its crude. TLC system: MeOH/CH₂Cl₂ (10:90), R_{f} value=0.4; LCMS (ESI) m/z 450.2 [C₂₃H₃₅NO₆Si+H]+. The crude compound was used in the next step without further purification.

### Synthesis of tert-butyl (2-((tert-butyldimethylsilyl)oxy)ethyl)((2-(piperidine-1-carbonyl)benzofuran-6-yl)methyl)carbamate (D-12)

To a stirred solution of D-11 (267 mg, 0.59 mmol, 1.0 equiv.) in DMF (2 mL) at RT, was added piperidine (87.9 μL, 0.89 mmol, 1.5 equiv.), HATU (338 mg, 0.89 mmol, 1.5 equiv.) and DIPEA (0.31 mL, 1.77 mmol, 3.0 equiv.). The resultant mixture was stirred at RT for 4 h. Upon the completion of reaction by TLC, the reaction mixture was quenched with H₂O (20 mL) and extracted with EtOAc (2×50 mL). The combined organic layers were dried over anhydrous Na₂SO₄, filtered, and concentrated under reduced pressure. The crude material obtained was purified by silica gel (60-120 mesh) column chromatography (elution with 30% EtOAc/hexane) to afford D-12 as a pale-yellow gummy (120 mg, yield: 37% over 2 steps). TLC system: MeOH/CH₂Cl₂ (10:90), R_{f} value=0.6; ¹H NMR (400 MHz, CDCl₃) δ ppm: 7.56 (d, J=8.0 Hz, 1H), 7.41-7.37 (m, 1H), 7.21-7.14 (m, 2H), 4.65 (s, 2H), 3.75-3.69 (m, 6H), 3.36-3.26 (m, 2H), 1.72-1.68 (m, 6H), 1.51-1.43 (d, 9H), 0.89 (s, 9H), 0.05 (s, 6H); LCMS (ESI) m/z 517.3 [C₂₈H₄₄N₂O₅Si+H]⁺.

### Synthesis of (6-(((2-hydroxyethyl)amino)methyl)benzofuran-2-yl)(piperidin-1-yl)methanone as formic acid salt (Compound 34)

To a stirred solution of D-12 (240 mg, 0.46 mmol, 1.0 equiv.) in 1,4-Dioxane (2.0 mL) at 0° C., was added 4 M HCl in 1,4-Dioxane (0.4 mL). The resultant mixture was warmed to RT and stirred at RT for 2 h. Upon the completion of reaction by TLC, the reaction mixture was neutralized with aq. NaHCO₃ solution and extracted with CH₂Cl₂ (2×40 mL). The combined organic layers were dried over anhydrous Na₂SO₄, filtered, and concentrated under reduced pressure. The crude product was purified by reverse phase column chromatography (Grace column, elution with 17% MeCN/0.1% FA in H₂O) to afford compound 34 (formic acid salt) as an off-white semi solid (85 mg, yield: 53%). TLC system: MeOH/CH₂Cl₂ (10:90), R_{f} value=0.1; ¹H NMR (400 MHz, DMSO-d₆) δ ppm: 8.23 (s, 1H; HCOOH), 7.67 (d, J=8.4 Hz, 1H), 7.64 (s, 1H), 7.34-7.31 (m, 2H), 3.92 (s, 2H), 3.64 (br. s, 4H), 3.50 (t, J=5.6 Hz, 2H), 2.64 (t, J=5.6 Hz, 2H), 1.66-1.64 (m, 2H), 1.60-1.54 (m, 4H); LCMS (ESI) m/z 303.1 [C₁₇H₂₂N₂O₃+H]⁺.

### Example 11: Synthesis of Compound 16 Via Synthesis Route E

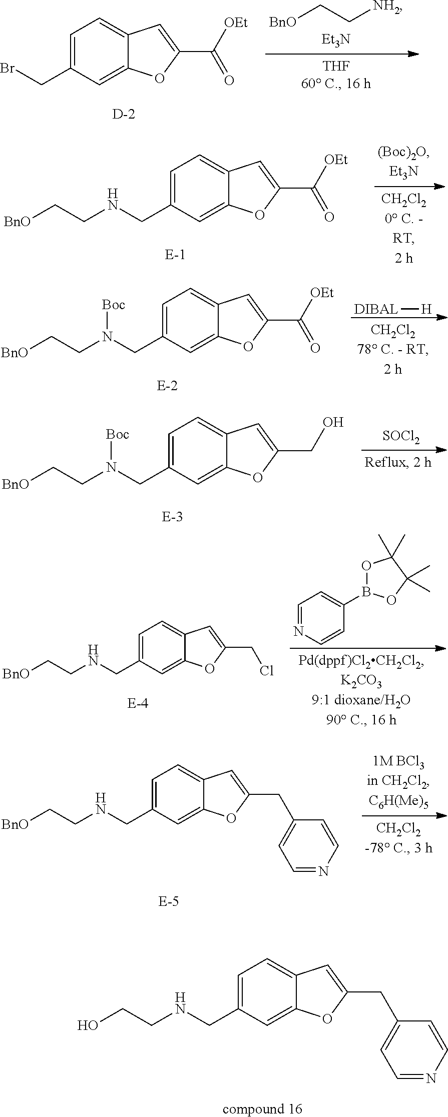

### Synthesis of ethyl 6-(((2-(benzyloxy)ethyl)amino)methyl)benzofuran-2-carboxylate (E-1)

Compound E-1 was obtained following the alkylation procedure similar to that for the synthesis of compound D-3, utilizing 2-(benzyloxy)ethan-1-amine (1.2 equiv.) with Et₃N (2.0 equiv.) and reaction conditions of 60° C. for 16 h. Compound E-1 was obtained in 60% yield as a brown gum following silica gel (60-120 mesh) column chromatography (elution with 20% EtOAc/hexane). TLC system: EtOAc/hexane (20:80), R_{f} value=0.2; ¹H NMR (400 MHz, CDCl₃) δ ppm: 7.65 (d, J=8.0 Hz, 1H), 7.60 (s, 1H), 7.54 (s, 1H), 7.41-7.36 (m, 4H), 7.34-7.30 (m, 2H), 4.57 (s, 2H), 4.48 (q, J=7.2 Hz, 2H), 3.97 (s, 2H), 3.66 (t, J=5.2 Hz, 2H), 2.89 (t, J=5.2 Hz, 2H), 1.47 (t, J=7.2 Hz, 3H).

### Synthesis of ethyl 6-(((2-(benzyloxy)ethyl)(tert-butoxycarbonyl)amino)methyl)benzofuran-2-carboxylate (E-2)

Compound E-2 was obtained following the Boc protection procedure similar to that for the synthesis of compound D-4, utilizing (Boc)₂O (1.2 equiv.) with Et₃N (1.5 equiv.) and reaction conditions of 0° C. to RT for 2 h. Compound E-2 was obtained in 78% yield as a brown oil following silica gel (60-120 mesh) column chromatography (elution with 10% EtOAc/hexane). TLC system: EtOAc/hexane (20:80), R_{f} value=0.5; ¹H NMR (400 MHz, CDCl₃) δ ppm: 7.59 (d, J=8.0 Hz, 1H), 7.50 (s, 1H), 7.45 (br. s, 1H), 7.35-7.28 (m, 5H), 7.20-7.17 (m, 1H), 4.67-4.65 (m, 2H), 4.47-4.42 (m, 4H), 3.64-3.37 (m, 4H), 1.47-1.41 (m, 12H); LCMS (ESI) m/z 454.2 [C₂₆H₃₁NO₆+H]⁺.

### Synthesis of tert-butyl (2-(benzyloxy)ethyl)((2-(hydroxymethyl)benzofuran-6-yl)methyl)carbamate (E-3)

Compound E-3 was obtained following the ester reduction procedure similar to that for the synthesis of compound D-5, utilizing DIBAL-H (2.0 equiv.) and reaction conditions of −78° C. to RT for 2 h. Compound E-3 was obtained in 77% yield as a yellow oil following silica gel (60-120 mesh) column chromatography (elution with 20% EtOAc/hexane). TLC system: EtOAc/hexane (30:70), R_{f} value=0.4; ¹H NMR (400 MHz, CDCl₃) δ ppm: 7.46 (d, J=8.0 Hz, 1H), 7.35-7.27 (m, 6H), 7.11-7.09 (m, 1H), 6.63 (s, 1H), 4.76 (d, J=5.6 Hz, 2H), 4.63 (s, 2H), 4.48 (s, 2H), 3.63-3.53 (m, 2H), 3.47-3.45 (m, 2H), 1.94 (t, J=6.0 Hz, 1H), 1.46 (s, 9H); LCMS (ESI) m/z 412.2 [C₂₄H₂₉NO₅+H]⁺.

### Synthesis of 2-(benzyloxy)-N-((2-(chloromethyl)benzofuran-6-yl)methyl)ethan-1-amine (E-4)

A solution of E-3 (900 mg, 2.19 mmol, 1.0 equiv.) in SOCl₂ (0.9 mL) was stirred at 80° C. for 2 h. Upon the completion of reaction by TLC, the volatiles were removed under reduced pressure and co-distilled with toluene to afford E-4 as a brown oil (980 mg) in its crude. TLC system: EtOAc/hexane (20:80), R_{f} value=0.6; ¹H NMR (400 MHz, CDCl₃) δ ppm: 7.67 (s, 1H), 7.54 (d, J=7.6 Hz, 1H), 7.44 (d, J=7.6 Hz, 1H), 7.35-7.28 (m, 5H), 6.69 (s, 1H), 4.64 (s, 2H), 4.55 (s, 2H), 4.24 (br. s, 2H), 3.85 (br. s, 2H), 3.06 (br. s, 2H). The crude material was used in the next step without purification.

### Synthesis of 2-(benzyloxy)-N-((2-(pyridin-4-ylmethyl)benzofuran-6-yl)methyl)ethan-1-amine (E-5)

To a stirred solution of E-4 (722 mg, 2.19 mmol, 1.0 equiv.) in 1:9 H₂O/1,4-dioxane (10 mL) at RT, was added 4-(4,4,5,5-tetramethyl-1,3,2-dioxaborolan-2-yl)pyridine (539 mg, 2.63 mmol, 1.2 equiv.) and K₂CO₃ (605 mg, 4.38 mmol, 2.0 equiv.). The reaction mixture was degassed for 10 min before Pd(dppf)Cl₂·DCM (180 mg, 0.22 mmol, 0.1 equiv.) was added. The resultant mixture was allowed to stir at 90° C. for 16 h, and upon the completion of reaction by TLC, the reaction mixture was diluted with H₂O (50 mL) and extracted with EtOAc (3×50 mL). The combined organic layers were dried over anhydrous Na₂SO₄, concentrated under reduced pressure, and purified by reverse phase column chromatography (Grace column, elution with 11% MeCN/0.01% FA in H₂O) to afford E-5 as an off-white solid (330 mg, yield: 40% over 2 steps). TLC system: MeOH/CH₂Cl₂ (05:95), R_{f} value=0.4; ¹H NMR (400 MHz, CDCl₃) δ ppm: 8.54 (d, J=4.8 Hz, 2H), 7.43 (d, J=8.0 Hz, 1H), 7.39 (br. s, 1H), 7.34-7.31 (m, 4H), 7.29-7.28 (m, 1H), 7.22 (d, J=6.0 Hz, 2H), 7.16 (dd, J=8.0 Hz, 1.2 Hz, 1H), 6.43 (s, 1H), 4.52 (s, 2H), 4.10 (s, 2H), 3.89 (s, 2H), 3.62 (t, J=5.2 Hz, 2H), 2.84 (t, J=5.2 Hz, 2H); LCMS (ESI) m/z 373.2 [C₂₄H₂₄N₂O₂+H]⁺.

### Synthesis of 2-(((2-(pyridin-4-ylmethyl)benzofuran-6-yl)methyl)amino)ethan-1-ol (Compound 16)

To a stirred solution of E-5 (320 mg, 0.86 mmol, 1.0 equiv.) in CH₂Cl₂ (3.2 mL) at −78° C., was added Me₅Ph (191 mg, 1.29 mmol, 1.5 equiv.) and BCl₃ (1 M in CH₂Cl₂) (2.58 mL, 2.58 mmol, 3.0 equiv.). The resultant mixture was stirred at −78° C. for 3 h and upon the completion of reaction by TLC, the reaction mixture was quenched with aq. NaHCO₃ solution and extracted with 5% MeOH in CH₂Cl₂ (2×30 mL). The combined organic layers were dried over anhydrous Na₂SO₄, concentrated under reduced pressure, and purified by Prep-HPLC (ammonium bicarbonate buffer) to afford compound 16 as a brown gummy solid (35 mg, yield: 14%). TLC system: MeOH/CH₂Cl₂ (5:95), R_{f} value=0.2; ¹H NMR (400 MHz, DMSO-d₆) δ ppm: 8.51 (d, J=6.0 Hz, 2H), 7.47 (d, J=8.0 Hz, 1H), 7.44 (s, 1H), 7.32 (d, J=5.6 Hz, 2H), 7.17 (d, J=8.0 Hz, 1H), 6.65 (s, 1H), 4.43 (br. s, 1H), 4.19 (s, 2H), 3.77 (s, 2H), 3.47-3.43 (m, 2H), 2.54 (t, J=5.6 Hz, 2H); LCMS (ESI) m/z 283.1 [C₁₇H₁₈N₂O₂+H]⁺.

### Example 12: Synthesis of Compound 18 Via Synthesis Route F

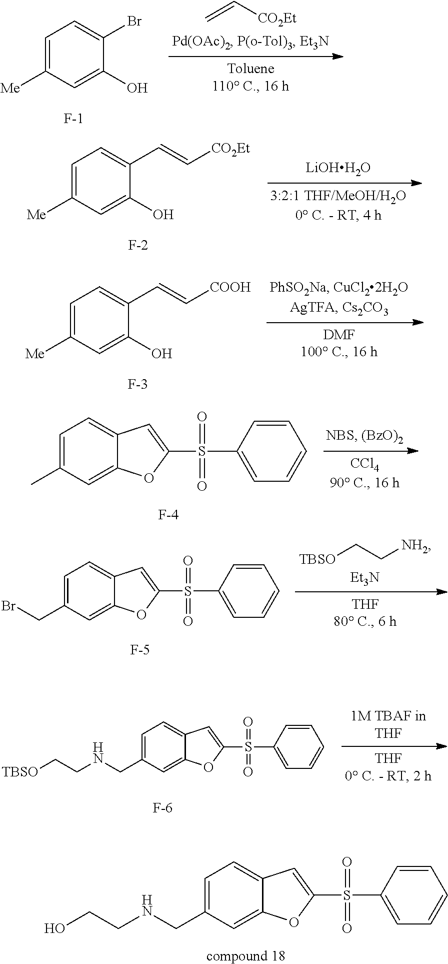

### Synthesis of ethyl (E)-3-(2-hydroxy-4-methylphenyl)acrylate (F-2)

In a sealed tube, to a degassed solution of 2-bromo-5-methylphenol F-1 (5.00 g, 26.7 mmol, 1.0 equiv.) in toluene (100 mL) at RT, was added Et₃N (11.2 mL, 80.1 mmol, 3.0 equiv.), ethyl acrylate (4.27 mL, 40.1 mmol, 1.5 equiv.), Pd(OAc)₂ (301 mg, 1.34 mmol, 0.05 equiv.) and P(o-tol)₃ (813 mg, 2.67 mmol, 0.1 equiv.). The resultant mixture was stirred at 110° C. for 16 h. Upon the completion of reaction by TLC, the reaction mixture was diluted with H₂O (100 mL) and extracted with EtOAc (2×200 mL). The combined organic layers were dried over anhydrous Na₂SO₄, filtered, and concentrated under reduced pressure. The crude product obtained was purified by silica gel (60-120 mesh) column chromatography (elution with 20% EtOAc/hexane) to afford F-2 as a brown solid (3.60 g, yield: 65%). TLC system: EtOAc/hexane (20:80), R_{f} value=0.3; ¹H NMR (400 MHz, CDCl₃) δ ppm: 7.94 (d, J=16.0 Hz, 1H), 7.36 (d, J=8.0 Hz, 1H), 6.75 (d, J=8.0 Hz, 1H), 6.63 (s, 1H), 6.54 (d, J=16.0 Hz, 1H), 5.66 (s, 1H), 4.27 (q, J=7.2 Hz, 2H), 2.31 (s, 3H), 1.34 (t, J=7.2 Hz, 3H); LCMS (ESI) m/z 207.1 [C₁₂H₁₄O₃+H]⁺.

### Synthesis of (E)-3-(2-hydroxy-4-methylphenyl)acrylic acid (F-3)

To a solution of F-2 (3.60 g, 17.5 mmol, 1.0 equiv.) in 3:2:1 THF/MeOH/H₂O (21.6 mL) at 0° C., was added LiOH·H₂O (2.94 g, 70.0 mmol, 4.0 equiv.). The resultant mixture was warmed to RT and stirred at RT for 4 h. Upon the completion of reaction by TLC, the reaction mixture was evaporated under reduced pressure, acidified with 1N HCl solution and extracted with 10% MeOH/CH₂Cl₂ (3×50 mL). The combined organic layers were dried over anhydrous Na₂SO₄, filtered, and concentrated under reduced pressure to afford F-3 as an off-white solid (2.51 g, yield: 80%). TLC system: EtOAc/hexane (100:0), R_{f} value=0.1; ¹H NMR (400 MHz, CDCl₃) δ ppm: 7.99 (d, J=16.0 Hz, 1H), 7.38 (d, J=8.0 Hz, 1H), 6.78 (d, J=7.6 Hz, 1H), 6.62 (s, 1H), 6.53 (d, J=16.0 Hz, 1H), 2.32 (s, 3H); LCMS (ESI) m/z 179.2 [C₁₀H₁₀O₃+H]⁺.

### Synthesis of 6-methyl-2-(phenylsulfonyl)benzofuran (F-4)

To a stirred solution of F-3 (2.50 g, 14.0 mmol, 1.0 equiv.) in DMF (100 mL) at RT, was added sodium benzenesulfinate (4.60 g, 28.0 mmol, 2.0 equiv.), Cs₂CO₃ (10.0 g, 30.8 mmol, 2.2 equiv.), AgTFA (7.73 g, 35.0 mmol, 2.5 equiv.) and CuCl₂·2H₂O (1.43 g, 8.40 mmol, 0.6 equiv.). The resultant mixture was heated to 100° C. and stirred at 100° C. for 16 h. Upon the completion of reaction by TLC, the reaction mixture was concentrated and the crude obtained was purified by silica gel (100-200 mesh) column chromatography (elution with 20% EtOAc/hexane) to afford F-4 as a brown solid (1.00 g, yield: 26%). TLC system: EtOAc/hexane (20:80), R_{f} value=0.8; LCMS (ESI) m/z 273.0 [C₁₅H₁₂O₃S+H]⁺.

### Synthesis of 6-(bromomethyl)-2-(phenylsulfonyl)benzofuran (F-5)

Compound F-5 was obtained following the bromination procedure similar to that for the synthesis of compound D-2, utilizing NBS (1.7 equiv.) with benzoic peroxyanhydride (0.1 equiv.) and reaction conditions of 90° C. for 16 h. Compound F-5 was obtained as a brown solid following silica gel (60-120 mesh) column chromatography (elution with 20% EtOAc/hexane) as a semi-pure material. TLC system: EtOAc/hexane (20:80), R_{f} value=0.4. The semi-pure material was used in the next step without further purification.

### Synthesis of 2-((tert-butyldimethylsilyl)oxy)-N-((2-(phenylsulfonyl)benzofuran-6-yl)methyl)ethan-1-amine (F-6)

Compound F-6 was obtained following the alkylation procedure similar to that for the synthesis of compound D-3, utilizing 2-((tert-butyldimethylsilyl)oxy)ethan-1-amine (1.3 equiv.) with Et₃N (2.0 equiv.) and reaction conditions of 80° C. for 6 h. Compound F-6 was obtained in 20% yield (over 2 steps) as a brown gummy liquid following silica gel (60-120 mesh) column chromatography (elution with 50% EtOAc/hexane). TLC system: EtOAc/hexane (50:50), R_{f} value=0.3; ¹H NMR (400 MHz, CDCl₃) δ ppm: 8.08 (d, J=7.6 Hz, 2H), 7.65-7.62 (m, 2H), 7.60-7.51 (m, 4H), 7.31-7.29 (m, 1H), 3.93 (s, 2H), 3.72 (t, J=5.2 Hz, 2H), 2.71 (t, J=5.2 Hz, 2H), 0.87 (s, 9H), 0.04 (s, 6H); LCMS (ESI) m/z 446.2 [C₂₃H₃₁NO₄SSi+H]⁺.

### Synthesis of 2-(((2-(phenylsulfonyl)benzofuran-6-yl)methyl)amino)ethan-1-ol as Formic Acid Salt (Compound 18)

To a stirred solution of F-6 (420 mg, 0.94 mmol, 1.0 equiv.) in THE (1.0 mL) at 0° C., was added 1 M TBAF in THE (1.89 mL, 1.88 mmol, 2.0 equiv.). The resultant mixture was warmed to RT and stirred at RT for 2 h. Upon the completion of reaction by TLC, the reaction mixture was quenched with saturated aq. NaHCO₃ solution (20 mL) and extracted with CH₂Cl₂ (2×30 mL). The combined organic layers were washed with water (20 mL), followed by brine (20 mL), dried over Na₂SO₄, filtered, concentrated, and purified by reverse phase column chromatography (Grace column, elution with 26% MeCN/0.1% FA in H₂O) to afford compound 18 (formic acid salt) as a white solid (35 mg, yield: 10%). TLC system: MeOH/CH₂Cl₂ (20:80), R_{f} value=0.2; melting point range: 108-112° C.; ¹H NMR (400 MHz, DMSO-d₆) δ ppm: 8.16 (s, 1H; HCOOH), 8.05-8.03 (m, 2H), 7.93 (s, 1H), 7.81-7.78 (m, 2H), 7.75 (s, 1H), 7.72-7.69 (m, 2H), 7.46-7.44 (m, 1H), 4.01 (s, 2H), 3.52 (t, J=5.6 Hz, 2H), 2.69 (t, J=5.6 Hz, 2H); LCMS (ESI) m/z 332.2 [C₁₇H₁₇NO₄S+H]⁺.

### Example 13: Synthesis of Compound 19 Via Synthesis Route G

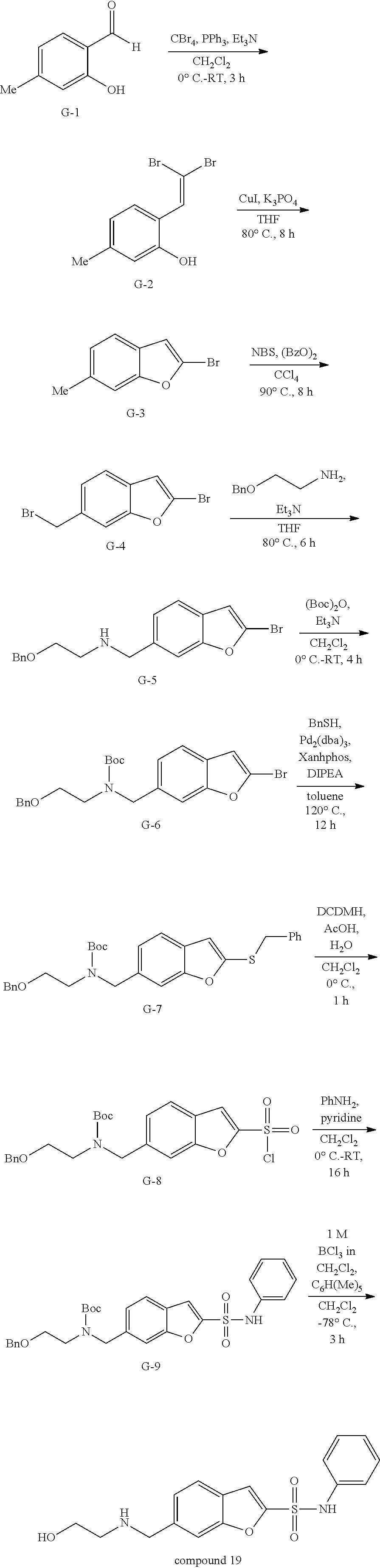

### Synthesis of 2-(2,2-dibromovinyl)-5-methylphenol (G-2)

To a stirred solution of CBr₄ (73.0 g, 220 mmol, 3.0 equiv.) in CH₂Cl₂ (700 mL) at 0° C., was added triphenylphosphine (57.7 g, 220 mmol, 3.0 equiv.). The mixture was stirred at 0° C. for 10 min before Et₃N (61.3 mL, 440 mmol, 6.0 equiv.) was added dropwise. The mixture was stirred for 10 min before a solution of benzaldehyde G-1 (10.0 g, 73.4 mmol, 1.0 equiv.) in CH₂Cl₂ (100 mL) was added dropwise. The resultant mixture was stirred at 0° C. for 1 h and warmed to RT and stirred at RT for 2 h. Upon the completion of reaction by TLC, the reaction mixture was diluted with H₂O (200 mL) and extracted with CH₂Cl₂ (2×300 mL). The combined organic layers were dried over anhydrous Na₂SO₄ and concentrated under reduced pressure. The crude compound obtained was purified by silica gel (60-120 mesh) column chromatography (elution with 10% EtOAc/hexane) to afford G-2 as a brown solid (11.5 g, yield: 54%). TLC system: EtOAc/hexane (10:90), R_{f} value=0.8; ¹H NMR (400 MHz, CDCl₃) δ ppm: 7.53 (s, 1H), 7.45 (d, J=7.6 Hz, 1H), 6.76 (d, J=8.0 Hz, 1H), 6.64 (s, 1H), 5.05 (s, 1H), 2.29 (s, 3H).

### Synthesis of 2-bromo-6-methylbenzofuran (G-3)

To a stirred solution of G-2 (11.5 g, 39.4 mmol, 1.0 equiv.) in THE (115 mL) at RT, was added CuI (750 mg, 3.94 mmol, 0.1 equiv.) and K₃PO₄ (16.7 g, 78.8 mmol, 2.0 equiv.). The resultant mixture was stirred at 80° C. for 8 h and upon the completion of reaction by TLC, the reaction mixture was diluted with H₂O (200 mL) and extracted with CH₂Cl₂ (2×150 mL). The combined organic layers were dried over anhydrous Na₂SO₄, filtered, and concentrated under reduced pressure. The crude compound obtained was purified by silica gel (60-120 mesh) column chromatography (elution with 10% EtOAc/hexane) to afford G-3 as a brown oil (8.00 g, yield: 96%). TLC system: EtOAc/hexane (10:90), R_{f} value=0.8; ¹H NMR (400 MHz, CDCl₃) δ ppm: 7.36 (d, J=7.6 Hz, 1H), 7.25 (m, 1H), 7.04 (d, J=8.0 Hz, 1H), 6.66 (s, 1H), 2.45 (s, 3H).

### Synthesis of 2-bromo-6-(bromomethyl)benzofuran (G-4)

Compound G-4 was obtained following the bromination procedure similar to that for the synthesis of compound D-2, utilizing NBS (1.5 equiv.) with benzoic peroxyanhydride (0.1 equiv.) and reaction conditions of 90° C. for 8 h. Compound G-4 was obtained as a brown solid as a crude material. TLC system: MeOH/CH₂Cl₂ (10:90), R_{f} value=0.7; ¹H NMR (400 MHz, CDCl₃) δ ppm: 7.50-7.43 (m, 2H), 7.29-7.27 (m, 1H), 6.72 (s, 1H), 4.61 (s, 2H). The crude material was used in the next step without further purification.

### Synthesis of 2-(benzyloxy)-N-((2-bromobenzofuran-6-yl)methyl)ethan-1-amine (G-5)

Compound G-5 was obtained following the alkylation procedure similar to that for the synthesis of compound D-3, utilizing 2-(benzyloxy)ethan-1-amine (1.5 equiv.) with Et₃N (2.0 equiv.) and reaction conditions of 80° C. for 6 h. Compound G-5 was obtained in 46% yield (over 2 steps) as a brown oil following silica gel (60-120 mesh) column chromatography (elution with 50% EtOAc/hexane). TLC system: EtOAc/hexane (50:50), R_{f} value=0.2; ¹H NMR (400 MHz, CDCl₃) δ ppm: 7.44-7.39 (m, 2H), 7.35-7.28 (m, 5H), 7.19 (d, J=8.8 Hz, 1H), 6.69 (s, 1H), 4.52 (s, 2H), 3.89 (s, 2H), 3.62 (t, J=5.2 Hz, 2H), 2.85 (t, J=5.2 Hz, 2H); LCMS (ESI) m/z 362.1 [C₁₈H₁₈BrNO₂+H]⁺.

### Synthesis of tert-butyl (2-(benzyloxy)ethyl)((2-bromobenzofuran-6-yl)methyl)carbamate (G-6)

Compound G-6 was obtained following the Boc protection procedure similar to that for the synthesis of compound D-4, utilizing (Boc)₂O (1.5 equiv.) with Et₃N (2.0 equiv.) and reaction conditions of 0° C. to RT for 4 h. Compound G-6 was obtained in 79% yield as a brown oil following silica gel (60-120 mesh) column chromatography (elution with 20% EtOAc/hexane). TLC system: EtOAc/hexane (20:80), R_{f} value=0.5; ¹H NMR (400 MHz, CDCl₃) δ ppm: 7.41 (d, J=8.0 Hz, 1H), 7.39-7.35 (m, 1H), 7.33-7.29 (m, 5H), 7.13-7.10 (m, 1H), 6.69 (s, 1H), 4.62 (s, 2H), 4.48 (s, 2H), 3.63-3.53 (m, 2H), 3.47-3.35 (m, 2H), 1.46 (s, 9H); LCMS (ESI) m/z 462.1 [C₂₃H₂₆BrNO₄+H]⁺.

### Synthesis of tert-butyl (2-(benzyloxy)ethyl)((2-(benzylthio)benzofuran-6-yl)methyl)carbamate (G-7)

To a stirred solution of G-6 (2.50 g, 5.43 mmol, 1.0 equiv.) in toluene (25 mL) at RT, was added DIPEA (1.90 mL, 10.9 mmol, 2.0 equiv.) and phenylmethanethiol (0.83 mL, 7.06 mmol, 1.3 equiv.). The reaction mixture was purged with argon gas for 10 min before Pd₂(dba)₃ (247 mg, 0.27 mmol, 0.05 equiv.) and Xanthphos (312 mg, 0.54 mmol, 0.1 equiv.) were added. The resultant reaction mixture was stirred at 120° C. for 12 h. Upon the completion of reaction by TLC, the reaction mixture was diluted with H₂O (80 mL) and extracted with EtOAc (2×80 mL). The combined organic layers were dried over anhydrous Na₂SO₄, filtered, and concentrated under reduced pressure. The crude compound obtained was purified by silica gel (60-120 mesh) column chromatography (elution with 30% EtOAc/hexane) to afford G-7 as a brown oil (1.40 g, yield: 51%). TLC system: EtOAc/hexane (20:80), R_{f} value=0.5; ¹H NMR (400 MHz, CDCl₃) δ ppm: 7.38-7.29 (m, 8H), 7.23-7.16 (m, 5H), 6.63 (s, 1H), 4.62 (br. s, 2H), 4.49 (br. s, 2H), 4.14 (s, 2H), 3.64-3.55 (m, 2H), 3.48-3.38 (m, 2H), 1.44 (s, 9H); LCMS (ESI) m/z 504.3 [C₃₀H₃₃NO₄S+H]⁺.

### Synthesis of tert-butyl (2-(benzyloxy)ethyl)((2-(chlorosulfonyl)benzofuran-6-yl)methyl)carbamate (G-8)

To a stirred solution of G-7 (1.00 g, 1.99 mmol, 1.0 equiv.) in CH₂Cl₂ (10 mL) at 0° C., was added AcOH (2.5 mL), H₂O (7.5 mL) and DCDMH (1.96 g, 9.95 mmol, 5 equiv.). The resultant reaction mixture was stirred at 0° C. for 1 h. Upon the completion of reaction by TLC, the reaction mixture was diluted with H₂O (20 mL) and extracted with CH₂Cl₂ (2×40 mL). The combined organic layers were dried over anhydrous Na₂SO₄, filtered, and concentrated under reduced pressure to afford G-8 as a brown gum (2 g, crude). TLC system: EtOAc/hexane (10:90), R_{f} value=0.6. The crude material was used in the next step immediately.

### Synthesis of tert-butyl (2-(benzyloxy)ethyl)((2-(N-phenylsulfamoyl)benzofuran-6-yl)methyl)carbamate (G-9)

To a stirred solution of G-8 (2 g crude, 4.17 mmol, 1.0 equiv.) in CH₂Cl₂ (20 mL) at 0° C., was added pyridine (0.67 mL, 8.34 mmol, 2 equiv.) and aniline (0.76 mL, 8.34 mmol, 2 equiv.). The resultant mixture was warmed to RT and stirred at RT for 16 h. Upon the completion of reaction by TLC, the reaction mixture was diluted with H₂O (20 mL) and extracted with CH₂Cl₂ (2×40 mL). The combined organic layers were dried over anhydrous Na₂SO₄, filtered, and concentrated under reduced pressure. The crude compound obtained was purified by silica gel (60-120 mesh) column chromatography (elution with 20% EtOAc/hexane) to afford G-9 as a pale-brown gum (260 mg, 64% pure by LCMS) as a semi-pure material. TLC system: EtOAc/hexane (30:70), R_{f} value=0.4; LCMS (ESI) m/z 535.5 [C₂₉H₃₂N₂O₆S−H]⁻. The semi-pure material was used in the next step without further purification.

Synthesis of 6-(((2-hydroxyethyl)amino)methyl)-N-phenylbenzofuran-2-sulfonamide as formic acid salt (compound 19): To a stirred solution of G-9 (260 mg, 0.48 mmol, 1.0 equiv.) in CH₂Cl₂ (2.60 mL) at −78° C., was added Me₅Ph (107 mg, 0.72 mmol, 1.5 equiv.) and BCl₃ (1 M in CH₂Cl₂) (1.0 mL, 0.96 mmol, 2.0 equiv.). The reaction mixture was stirred at −78° C. for 3 h. Upon the completion of reaction by TLC, the reaction mixture was quenched with MeOH (3 mL) and concentrated under reduced pressure. The crude compound obtained was purified by reverse phase column chromatography (Grace column, elution with 18% MeCN/0.1% FA in H₂O) to afford compound 19 (formic acid salt) as a light brown semi solid (50 mg, yield: 6% in three steps). TLC system: MeOH/CH₂Cl₂ (20:80), R_{f} value=0.3; H NMR (400 MHz, DMSO-d₆) δ ppm: 8.20 (s, 1H; HCOOH), 7.70-7.68 (m, 2H), 7.49 (s, 1H), 7.36 (d, J=9.2 Hz, 1H), 7.22-7.18 (m, 2H), 7.12-7.10 (m, 2H), 6.96 (t, J=7.2 Hz, 1H), 3.98 (s, 2H), 3.52 (t, J=5.6 Hz, 2H), 2.69 (t, J=5.6 Hz, 2H); LCMS (ESI) m/z 347.1 [C₁₇H₁₈N₂O₄S+H]⁺.

### Example 14: Synthesis of Compound 29 Via Synthesis Route H

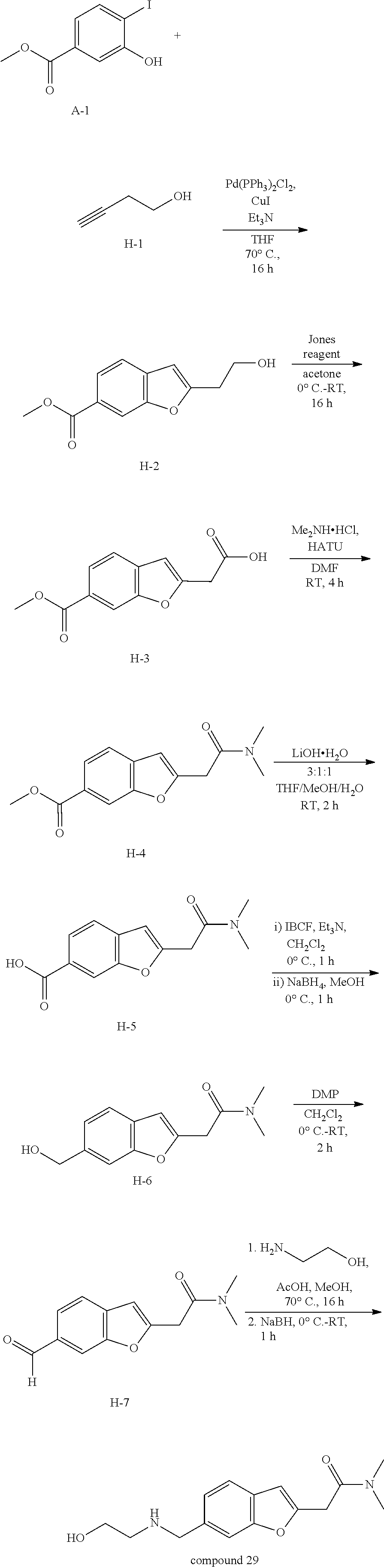

### Synthesis of methyl 2-(2-hydroxyethyl)benzofuran-6-carboxylate (H-2)

Compound H-2 was obtained following the Pd-catalyzed benzofuran ring formation procedure similar to that for the synthesis of compound A-3, utilizing but-3-yn-1-ol H-1 (1.2 equiv.) and reaction conditions of 70° C. for 16 h. Compound H-2 was obtained in 76% yield as a brick coloured solid following silica gel (100-200 mesh) column chromatography (elution with 60% EtOAc/hexane). TLC system: EtOAc/hexane (60:40), R_{f} value=0.3; ¹H NMR (400 MHz, CDCl₃) δ ppm: 8.11 (s, 1H), 7.92 (dd, J=8.0 Hz, 1.2 Hz, 1H), 7.52 (d, J=8.0 Hz, 1H), 6.57 (s, 1H), 4.03 (q, J=5.6 Hz, 2H), 3.94 (s, 3H), 3.08 (t, J=5.6 Hz, 2H), 1.66 (t, 1H); LCMS (ESI) m/z 221.1 [C₁₂H₁₂O₄+H]⁺.

### Synthesis of 2-(6-(methoxycarbonyl)benzofuran-2-yl)acetic acid (H-3)

To a stirred solution of H-2 (1.50 g 6.81 mmol, 1.0 equiv.) in dry acetone (15 mL) at 0° C., was added Jones reagent (1.5 mL). The resultant mixture was warmed to RT and stirred at RT for 16 h. Upon the completion of reaction by TLC, acid-base treatment was given to the reaction mixture and the mixture was extracted with 10% MeOH in CH₂Cl₂. The combined organic layers were dried over anhydrous Na₂SO₄ and concentrated under reduced pressure to afford H-3 as an orange coloured solid (750 mg, yield: 47%). TLC system: MeOH/CH₂Cl₂ (10:90), R_{f} value=0.1; ¹H NMR (400 MHz, CDCl₃) δ ppm: 8.14 (s, 1H), 7.94 (dd, J=8.0 Hz, 1.2 Hz, 1H), 7.56 (d, J=8.0 Hz, 1H), 6.74 (s, 1H), 3.94 (s, 3H), 3.93 (s, 2H); LCMS (ESI) m/z 235.1 [C₁₂H₁₀O₅+H]⁺.

### Synthesis of methyl 2-(2-(dimethylamino)-2-oxoethyl)benzofuran-6-carboxylate (H-4)

To a stirred solution of H-3 (750 mg, 3.20 mmol, 1.0 equiv.) in DMF (7.50 mL) at RT was added dimethylamine hydrochloride (391 mg, 4.80 mmol, 1.5 equiv.), HATU (1.83 g, 4.80 mmol, 1.5 equiv.), and DIPEA (2.79 mL, 16 mmol, 5.0 equiv.). The resultant mixture was stirred at RT for 4 h. Upon the completion of reaction by TLC, the reaction mixture was quenched with H₂O (50 mL) and extracted with EtOAc (2×100 mL). The combined organic layers were dried over anhydrous Na₂SO₄, filtered, and concentrated under reduced pressure. The crude compound obtained was purified by silica gel (100-200 mesh) column chromatography (elution with 70% EtOAc/hexane) to afford H-4 as a colourless liquid (750 mg) as semi-pure material. TLC system: EtOAc/hexane (60:40), R_{f} value=0.2; ¹H NMR (400 MHz, CDCl₃) δ ppm: 8.12 (s, 1H), 7.92 (dd, J=8.4 Hz, 1.2 Hz, 1H), 7.54 (d, J=8.4 Hz, 1H), 6.67 (s, 1H), 3.94 (s, 3H), 3.92 (s, 2H), 3.14 (s, 3H), 3.02 (s, 3H); LCMS (ESI) m/z 262.2 [C₁₄H₁₅NO₄+H]⁺.

### Synthesis of 2-(2-(dimethylamino)-2-oxoethyl)benzofuran-6-carboxylic acid (H-5)

To a stirred solution of H-4 (750 mg, 2.87 mmol, 1.0 equiv.) in a mixture of 3:1:1 THF/MeOH/H₂O (7.5 mL) at RT, was added LiOH·H₂O (241 mg, 5.74 mmol, 2 equiv.). The resultant mixture was stirred at RT for 2 h and upon the completion of reaction by TLC, the reaction mixture was neutralized with 2N HCl solution and extracted with EtOAc (3×100 mL). The combined organic layers were washed with brine, dried over Na₂SO₄, and concentrated under reduced pressure to afford H-5 (500 mg) as a pale yellow solid in its crude. TLC system: MeOH/CH₂Cl₂ (10:90), R_{f} value=0.1; ¹H NMR (400 MHz, CDCl₃) δ ppm: 8.15 (s, 1H), 7.94 (dd, J=8.0 Hz, 1.2 Hz, 1H), 7.54 (d, J=8.0 Hz, 1H), 6.69 (d, J=0.8 Hz, 1H), 3.95 (s, 2H), 3.17 (s, 3H), 3.03 (s, 3H); LCMS (ESI) m/z 248.1 [C₁₃H₁₃NO₄+H]⁺. The crude material was used in the next step without further purification.

### Synthesis of 2-(6-(hydroxymethyl)benzofuran-2-yl)-N,N-dimethylacetamide (H-6)

To a stirred solution of H-5 (500 mg, 2.02 mmol, 1.0 equiv.) in CH₂Cl₂ (10 mL) at 0° C., was added Et₃N (0.37 mL, 2.63 mmol, 1.3 equiv.) and IBCF (0.39 mL, 3.03 mmol, 1.5 equiv.). The reaction mixture was warmed to RT and stirred at RT for 1 h. Upon the completion of reaction by TLC, H₂O (20 mL) was added and the mixture was extracted with CH₂Cl₂ (2×50 mL). The combined organic layers were dried over anhydrous Na₂SO₄, filtered, and concentrated under reduced pressure. The crude compound obtained was dissolved in MeOH (10 mL), cooled to 0° C., and NaBH₄ (191 mg, 5.05 mmol, 2.5 equiv.) was added. The resultant mixture was stirred at 0° C. for 1 h. Upon the completion of reaction by TLC, the reaction mixture was quenched with ice-cold water (30 mL) and extracted with CH₂Cl₂ (2×50 mL). The combined organic layers were dried over anhydrous Na₂SO₄, filtered, and concentrated under reduced pressure. The crude compound obtained was purified by silica gel (100-200 mesh) column chromatography (elution with 80% EtOAc/hexane) to afford H-6 as a yellow liquid (350 mg, yield: 47% over 3 steps). TLC system: EtOAc/hexane (60:40), R_{f} value=0.2; H NMR (400 MHz, CDCl₃) δ ppm: 7.48 (d, J=8.0 Hz, 1H), 7.46 (s, 1H), 7.20 (d, J=8.0 Hz, 1H), 6.58 (s, 1H), 4.78 (s, 2H), 3.89 (s, 2H), 3.12 (s, 3H), 3.01 (s, 3H), 1.68 (br. s, 1H); LCMS (ESI) m/z 234.2 [C₁₃H₁₅NO₃+H]⁺.

### Synthesis of 2-(6-formylbenzofuran-2-yl)-N,N-dimethylacetamide (H-7)

Compound H-7 was obtained following the alcohol oxidation procedure similar to that for the synthesis of compound A-5, utilizing DMP (1.5 equiv.) and reaction conditions of 0° C.-RT for 2 h. Compound H-7 was obtained in 72% yield as a yellow liquid following silica gel (100-200 mesh) column chromatography (elution with 60% EtOAc/hexane). TLC system: EtOAc/hexane (60:40), R_{f} value=0.3; ¹H NMR (400 MHz, CDCl₃) δ ppm: 10.05 (s, 1H), 7.94 (s, 1H), 7.76 (dd, J=8.0 Hz, 1.2 Hz, 1H), 7.63 (d, J=8.0 Hz, 1H), 6.71 (s, 1H), 3.94 (s, 2H), 3.15 (s, 3H), 3.02 (s, 3H); LCMS (ESI) m/z 232.1 [C₁₃H₁₃NO₃+H]⁺.

Synthesis of 2-(6-(((2-hydroxyethyl)amino)methyl)benzofuran-2-yl)-N,N-dimethylacetamide as formic acid salt (compound 29): Compound 29 was obtained following the reductive amination procedure similar to that for the synthesis of compound 2, utilizing 2-aminoethan-1-ol (1.5 equiv.), AcOH (0.1 equiv.), NaBH₄ (2.5 equiv.) and reaction conditions of 70° C. for 16 h for imine formation, and 0° C. to RT for 1 h for imine reduction. Compound 29 (formic acid salt) was obtained in 22% yield as a pale-yellow gummy liquid following reverse phase column chromatography (Grace column, elution with 25% MeCN/0.1% FA in H₂O). TLC system: MeOH/CH₂Cl₂ (10:90), R_{f} value=0.1; ¹H NMR (400 MHz, DMSO-d₆) δ ppm: 8.27 (s, 1H; HCOOH), 7.52-7.50 (m, 2H), 7.22 (d, J=8.4 Hz, 1H), 6.67 (s, 1H), 3.94 (s, 2H), 3.92 (s, 2H), 3.51 (t, J=5.6 Hz, 2H), 3.07 (s, 3H), 2.85 (s, 3H), 2.66 (t, J=5.6 Hz, 2H); LCMS (ESI) m/z 277.1 [C₁₅H₂₀N₂O₃+H]⁺.

### Example 15: Synthesis of Compound 38 Via Synthesis Route I

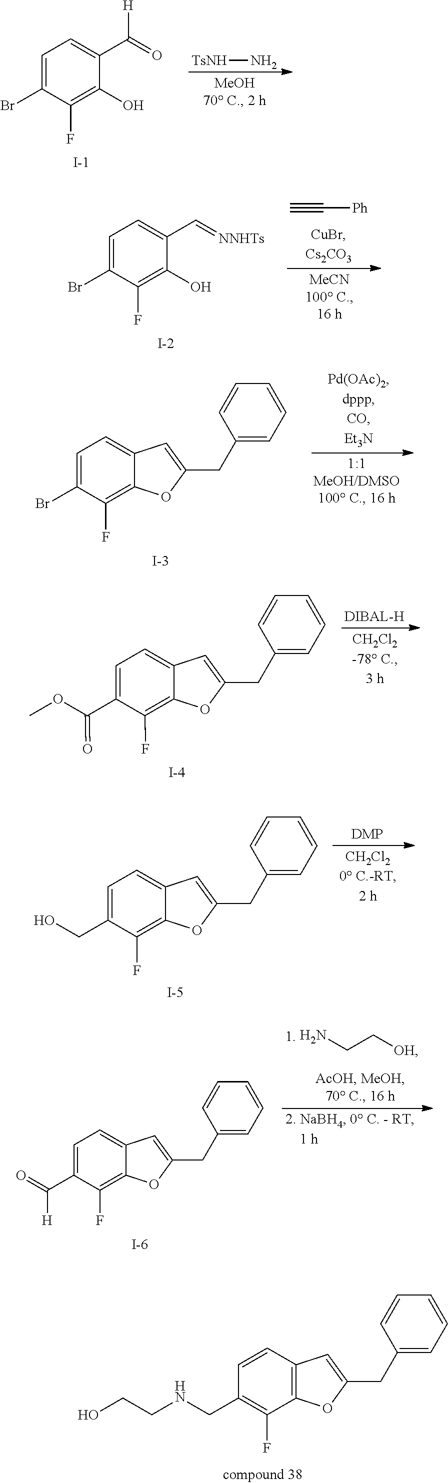

### Synthesis of N′-(4-bromo-3-fluoro-2-hydroxybenzylidene)-4-methylbenzenesulfonohydrazide (I-2)

To a stirred solution of benzaldehyde I-1 (1.00 g, 4.57 mmol, 1.0 equiv.) in MeOH (20 mL) at RT, was added tosyl hydrazine (765 mg, 4.11 mmol, 0.9 equiv.). The resultant mixture was stirred at 70° C. for 2 h. Upon the completion of reaction by TLC, the volatiles were removed under reduced pressure to afford I-2 (2.30 g) as a white solid in its crude. TLC system: EtOAc/hexane (50:50), R_{f} value=0.2; ¹H NMR (400 MHz, DMSO-d₆) δ ppm: 11.67 (br. s, 1H), 10.82 (s, 1H), 8.14 (s, 1H), 7.74 (d, J=8.4 Hz, 2H), 7.42 (d, J=8.0 Hz, 2H), 7.26 (dd, J=8.8 Hz, 1.6 Hz, 1H), 7.13 (dd, J=8.8 Hz, 6.4 Hz, 1H), 2.37 (s, 3H). The crude material was used in the next step without further purification.

### Synthesis of 2-benzyl-6-bromo-7-fluorobenzofuran (I-3)

To a stirred solution of I-2 (2.30 g, 5.94 mmol, 1.0 equiv.) and phenylacetylene (978 μL, 8.91 mmol, 1.5 equiv.) in MeCN (35 mL) at RT, was added Cs₂CO₃ (5.80 g, 17.8 mmol, 3.0 equiv.). The mixture was degassed for 10 min before CuBr (84.6 mg, 0.59 mmol, 0.1 equiv.) was added. The resultant mixture was stirred at 100° C. for 16 h. Upon the completion of reaction by TLC, the reaction mixture was diluted with H₂O (50 mL) and extracted with EtOAc (2×70 mL). The combined organic layers were dried over anhydrous Na₂SO₄, filtered, and concentrated under reduced pressure. The crude compound obtained was purified by silica gel (100-200 mesh) column chromatography (elution with 15% EtOAc/hexane) to afford I-3 (1.00 g, yield: 72% over 2 steps) as a pale-yellow solid. TLC system: EtOAc/hexane (30:80), R_{f} value=0.6; ¹H NMR (400 MHz, CDCl₃) δ ppm: 7.36-7.27 (m, 6H), 7.09 (d, J=8.4 Hz, 1H), 6.36-6.35 (m, 1H), 4.11 (s, 2H).

### Synthesis of methyl 2-benzyl-7-fluorobenzofuran-6-carboxylate (I-4)

To a stirred solution of I-3 (800 mg, 2.62 mmol, 1.0 equiv.) in 1:1 MeOH/DMSO (16 mL) at RT, was added dppp (214 mg, 0.52 mmol, 0.2 equiv.) and Et₃N (3.65 mL, 26.2 mmol, 10 equiv.). The mixture was degassed for 10 min before Pd(OAc)₂ (117 mg, 0.52 mmol, 0.2 equiv.) was added. The resultant mixture was stirred at 100° C. for 16 h under CO (100 psi) atmosphere. Upon the completion of reaction by TLC, the reaction mixture was diluted with H₂O (50 mL) and extracted with EtOAc (2×50 mL). The combined organic layers were dried over anhydrous Na₂SO₄, filtered, concentrated under reduced pressure, and purified by silica gel (100-200 mesh) column chromatography (elution with 10% EtOAc/hexane) to afford I-4 (570 mg, yield: 76%) as a pale-yellow solid. TLC system: EtOAc/hexane (20:80), R_{f} value=0.3; ¹H NMR (400 MHz, CDCl₃) δ ppm: 7.74 (dd, J=8.4 Hz, 6.0 Hz, 1H), 7.37-7.29 (m, 5H), 7.22 (d, J=8.0 Hz, 1H), 6.39 (d, J=3.2 Hz, 1H), 4.15 (s, 2H), 3.94 (s, 3H); LCMS (ESI) m/z 285.1 [C₁₇H₁₃FO₃+H]⁺.

### Synthesis of (2-benzyl-7-fluorobenzofuran-6-yl)methanol (I-5)

Compound I-5 was obtained following the ester reduction procedure similar to that for the synthesis of compound A-4, utilizing DIBAL-H (2.5 equiv.) and reaction conditions of −78° C. for 3 h. Compound I-5 was obtained as a crude and was used directly in the next step without further purification. TLC system: EtOAc/hexane (40:60), R_{f} value=0.3; ¹H NMR (400 MHz, CDCl₃) δ ppm: 7.36-7.27 (m, 5H), 7.22-7.17 (m, 2H), 6.36 (d, J=2.8 Hz, 1H), 4.83 (d, J=4.8 Hz, 2H), 4.13 (s, 2H), 1.73 (t, J=5.6 Hz, 1H); LCMS (ESI) m/z 239.1 [C₁₆H₁₃FO₂−OH]⁺.

### Synthesis of 2-benzyl-7-fluorobenzofuran-6-carbaldehyde (I-6)

Compound I-6 was obtained following the alcohol oxidation procedure similar to that for the synthesis of compound A-5, utilizing DMP (1.5 equiv.) and reaction conditions of 0° C.-RT for 2 h. Compound I-6 was obtained in 63% yield over 2 steps as a pale-yellow solid following silica gel (100-200 mesh) column chromatography (elution with 15% EtOAc/hexane). TLC system: EtOAc/hexane (20:80), R_{f} value=0.5; ¹H NMR (400 MHz, CDCl₃) δ ppm: 10.45 (s, 1H), 7.71 (dd, J=8.0 Hz, 5.6 Hz, 1H), 7.42-7.30 (m, 6H), 6.49 (d, J=2.8 Hz, 1H), 4.21 (s, 2H); LCMS (ESI) m/z 255.1 [C₁₆H₁₁FO₂+H]⁺.

Synthesis of 2-(((2-benzyl-7-fluorobenzofuran-6-yl)methyl)amino)ethan-1-ol as formic acid salt (compound 38): Compound 38 was obtained following the reductive amination procedure similar to that for the synthesis of compound 2, utilizing 2-aminoethan-1-ol (1.5 equiv.), AcOH (0.1 equiv.), NaBH₄ (2.5 equiv.) and reaction conditions of 70° C. for 16 h for imine formation, and 0° C. to RT for 1 h for imine reduction. Compound 38 (formic acid salt) was obtained in 74% yield as an off-white solid following reverse phase column chromatography (Grace column, elution with 17% MeCN/0.1% FA in H₂O). TLC system: MeOH/CH₂Cl₂ (10:90), R_{f} value=0.2; melting point range: 107-110° C.; ¹H NMR (400 MHz, DMSO-d₆) δ ppm: 8.26 (s, 1H; HCOOH), 7.37-7.30 (m, 5H), 7.28-7.26 (m, 2H), 6.70 (d, J=3.2 Hz, 1H), 4.18 (s, 2H), 3.96 (s, 2H), 3.51 (t, J=5.6 Hz, 2H), 2.68 (t, J=5.6 Hz, 2H); LCMS (ESI) m/z 300.2 [C₁₈H₁₈FNO₂+H]⁺.

### Example 16: Synthesis of Compound 42 Via Synthesis Route I

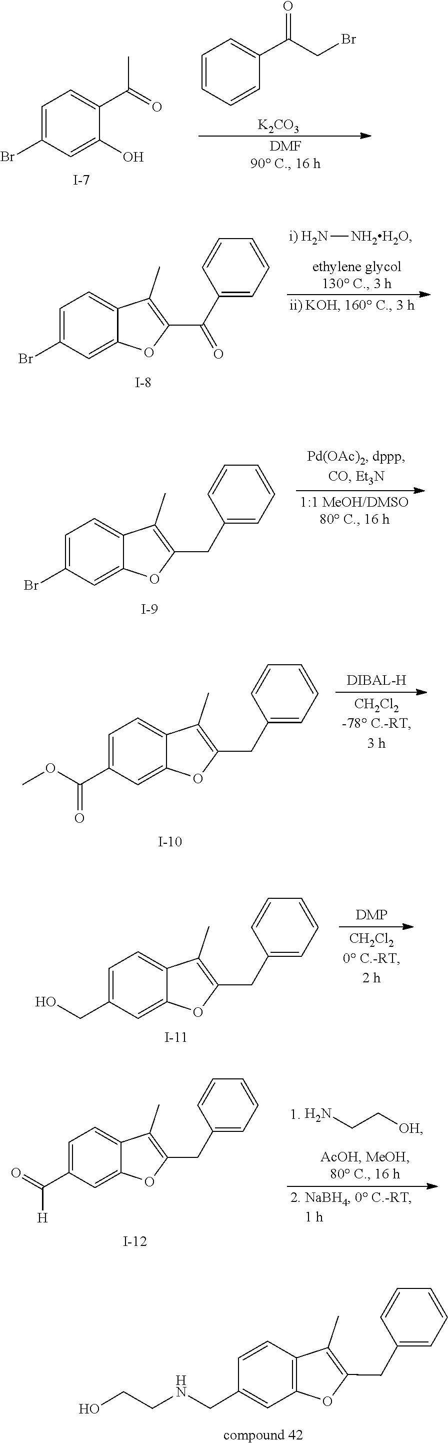

### Synthesis of (6-bromo-3-methylbenzofuran-2-yl)(phenyl)methanone (I-8)

To a stirred solution of I-7 (2.00 g, 9.30 mmol, 1.0 equiv.) in DMF (20 mL) at RT, was added K₂CO₃ (2.57 g, 18.6 mmol, 2.0 equiv.) and 2-bromo-1-phenylethan-1-one (2.23 g, 11.2 mmol, 1.2 equiv.). The resultant mixture was stirred at 90° C. for 16 h and upon the completion of reaction by TLC, the reaction mixture was diluted with H₂O and extracted with EtOAc (2×100 mL). The combined organic layers were dried over anhydrous Na₂SO₄, filtered, and concentrated under reduced pressure. The crude compound obtained was purified by silica gel (60-120 mesh) column chromatography (elution with 5% EtOAc/hexane) to afford I-8 (2.40 g, yield: 82%) as a brown solid. TLC system: EtOAc/hexane (5:95), R_{f} value=0.5; ¹H NMR (400 MHz, CDCl₃) δ ppm: 8.07-8.05 (m, 2H), 7.74 (d, J=1.6 Hz, 1H), 7.62-7.51 (m, 4H), 7.47 (dd, J=8.4 Hz, 1.6 Hz, 1H), 2.63 (s, 3H); LCMS (ESI) m/z 315.0, 317.0 [C₁₆H₁₁BrO₂+H]⁺.

### Synthesis of 2-benzyl-6-bromo-3-methylbenzofuran (I-9)

To a stirred solution of I-8 (2.00 g, 6.35 mmol, 1.0 equiv.) in ethylene glycol (25 mL) at RT, was added hydrazine monohydrate (1.27 g, 25.4 mmol, 4.0 equiv.). The reaction mixture was stirred at 130° C. for 3 h and upon the completion of reaction by TLC, the reaction mixture was cooled to RT before KOH (1.07 g, 19.1 mmol, 3.0 equiv.) was added. The resultant mixture was stirred at 160° C. for 3 h. Upon the completion of reaction by TLC, the reaction mixture was diluted with ice-cold water (100 mL) and extracted with EtOAc (2×100 mL). The combined organic layers were dried over anhydrous Na₂SO₄, filtered, and concentrated under reduced pressure. The crude compound obtained was purified by silica gel (60-120 mesh) column chromatography (elution with 5% EtOAc/hexane) to afford I-9 (1.50 g, yield: 78%) as a yellow solid. TLC system: EtOAc/hexane (5:95), R_{f} value=0.6; ¹H NMR (400 MHz, CDCl₃) δ ppm: 7.51 (s, 1H), 7.30-7.27 (m, 3H), 7.24-7.21 (m, 4H), 4.06 (s, 2H), 2.19 (s, 3H).

### Synthesis of methyl 2-benzyl-3-methylbenzofuran-6-carboxylate (I-10)

In a steel bomb, to a stirred solution of I-9 (1.00 g, 3.32 mmol, 1.0 equiv.) in 1:1 MeOH/DMSO (30 mL) at RT, was added Et₃N (4.63 mL, 33.2 mmol, 10 equiv.), Pd(OAc)₂ (148 mg, 0.66 mmol, 0.2 equiv.) and dppp (272 mg, 0.66 mmol, 0.2 equiv.). The resultant mixture was stirred at 80° C. for 16 h under CO atmosphere (100 psi). Upon the completion of reaction by TLC, the reaction mixture was diluted with H₂O (50 mL) and extracted with EtOAc (2×60 mL). The combined organic layers were dried over anhydrous Na₂SO₄, filtered, and concentrated under reduced pressure. The crude compound obtained was purified by silica gel (60-120 mesh) column chromatography (elution with 20% EtOAc/hexane) to afford I-10 (415 mg, yield: 45%) as a yellow gum. TLC system: EtOAc/hexane (5:95), R_{f} value=0.4; ¹H NMR (400 MHz, CDCl₃) δ ppm: 8.05 (s, 1H), 7.92 (dd, J=8.4 Hz, 1.2 Hz, 1H), 7.46 (d, J=8.0 Hz, 1H), 7.32-7.21 (m, 5H), 4.12 (s, 2H), 3.92 (s, 3H), 2.24 (s, 3H); LCMS (ESI) m/z 281.1 [C₁₈H₁₆O₃+H]⁺.

### Synthesis of (2-benzyl-3-methylbenzofuran-6-yl)methanol (I-11)

Compound I-11 was obtained following the ester reduction procedure similar to that for the synthesis of compound A-4, utilizing DIBAL-H (2.5 equiv.) and reaction conditions of −78° C. -RT for 2 h. Compound I-11 was obtained in 87% yield as a pale yellow solid following silica gel (60-120 mesh) column chromatography (elution with 20% EtOAc/hexane). TLC system: EtOAc/hexane (30:70), R_{f} value=0.5; ¹H NMR (400 MHz, CDCl₃) δ ppm: 7.43 (d, J=8.0 Hz, 1H), 7.39 (s, 1H), 7.31-7.27 (m, 2H), 7.25-7.19 (m, 4H), 4.76 (d, J=5.6 Hz, 2H), 4.09 (s, 2H), 2.22 (s, 3H), 1.60 (t, J=6.0 Hz, 1H); LCMS (ESI) m/z 235.1 [C₁₇H₁₆O₂−OH]⁺.

### Synthesis of 2-benzyl-3-methylbenzofuran-6-carbaldehyde (I-12)

Compound I-12 was obtained following the alcohol oxidation procedure similar to that for the synthesis of compound A-5, utilizing DMP (2.0 equiv.) and reaction conditions of 0° C.-RT for 2 h. Compound I-12 was obtained in 80% yield as a yellow gummy liquid following silica gel (60-120 mesh) column chromatography (elution with 15% EtOAc/hexane). TLC system: EtOAc/hexane (20:80), R_{f} value=0.7; ¹H NMR (400 MHz, CDCl₃) δ ppm: 10.03 (s, 1H), 7.86 (s, 1H), 7.76 (dd, J=8.0 Hz, 1.2 Hz, 1H), 7.55 (d, J=8.0 Hz, 1H), 7.33-7.22 (m, 5H), 4.14 (s, 2H), 2.26 (s, 3H); LCMS (ESI) m/z 251.1 [C₁₇H₁₄O₂+H]⁺.

### Synthesis of 2-(((2-benzyl-3-methylbenzofuran-6-yl)methyl)amino)ethan-1-ol as Formic Acid Salt (Compound 42)

Compound 42 was obtained following the reductive amination procedure similar to that for the synthesis of compound 2, utilizing 2-aminoethan-1-ol (2.0 equiv.), AcOH (0.1 equiv.), NaBH₄ (2.0 equiv.) and reaction conditions of 80° C. for 16 h for imine formation, and 0° C. to RT for 1 h for imine reduction. Compound 42 (formic acid salt) was obtained in 38% yield as an off-white gummy solid following reverse phase column chromatography (Grace column, elution with 28% MeCN/0.1% FA in H₂O). TLC system: MeOH/CH₂Cl₂ (10:90), R_{f} value=0.2; ¹H NMR (400 MHz, DMSO-d₆) δ ppm: 8.29 (s, 1H; HCOOH), 7.49-7.47 (m, 2H), 7.32-7.20 (m, 6H), 4.12 (s, 2H), 3.95 (s, 2H), 3.52 (t, J=5.6 Hz, 2H), 2.68 (t, J=5.6 Hz, 2H), 2.23 (s, 3H); LCMS (ESI) m/z 296.2 [C₁₉H₂₁NO₂+H]⁺.

### Example 17: Synthesis of Compound 43 Via Synthesis Route J

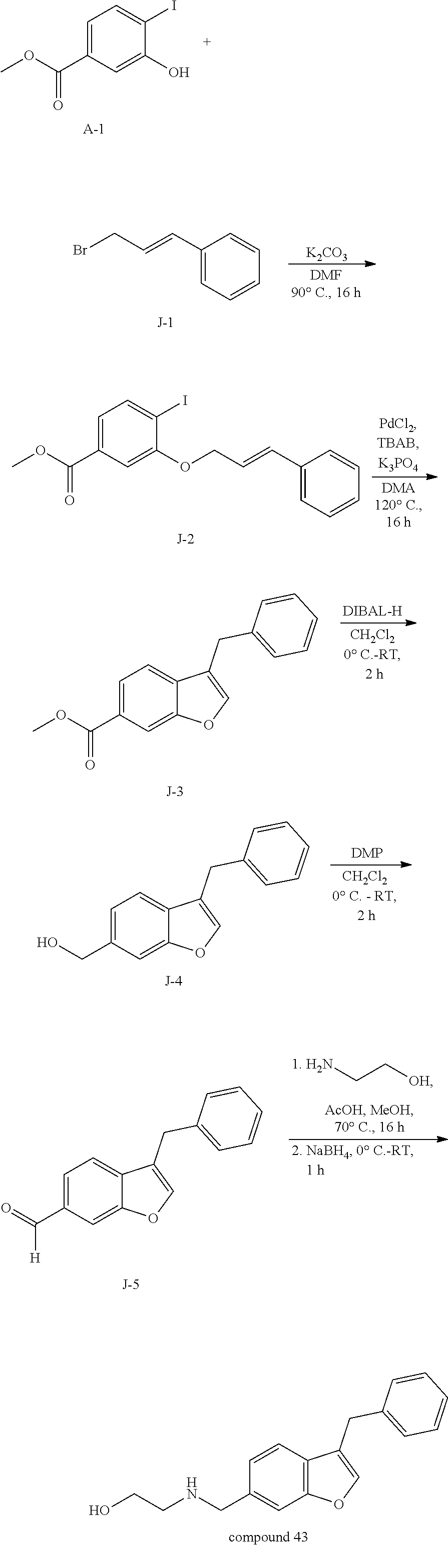

### Synthesis of methyl 3-(cinnamyloxy)-4-iodobenzoate (J-2)

To a stirred solution of methyl 3-hydroxy-4-iodobenzoate A-1 (1.00 g, 3.59 mmol, 1.0 equiv.) in DMF (10 mL) at RT, was added (E)-(3-bromoprop-1-en-1-yl)benzene J-1 (849 mg, 4.31 mmol, 1.2 equiv.) and K₂CO₃ (745 mg, 5.39 mmol, 1.5 equiv.). The resultant mixture was stirred at RT for 5 min before it was heated and stirred at 90° C. for 16 h. Upon the completion of reaction by TLC, the reaction mixture was quenched with H₂O (30 mL) and extracted with EtOAc (3×30 mL). The combined organic layers were washed with brine, dried over Na₂SO₄, filtered, and concentrated. The crude obtained was purified by silica gel (100-200 mesh) column chromatography (elution with 10% EtOAc/hexane) to afford J-2 (1.10 g, yield: 78%) as a brown oil. TLC system: EtOAc/hexane (20:80), R_{f} value=0.4; ¹H NMR (400 MHz, CDCl₃) δ ppm: 7.88 (d, J=8.4 Hz, 1H), 7.49 (d, J=1.6 Hz, 1H), 7.43 (d, J=7.2 Hz, 2H), 7.38 (dd, J=8.0 Hz, 2.0 Hz, 1H), 7.34 (t, J=7.2 Hz, 2H), 7.27 (d, J=7.2 Hz, 1H), 6.85 (d, J=16.0 Hz, 1H), 6.42 (dt, J=16.0 Hz, 5.2 Hz, 1H), 4.83 (dd, J=5.2 Hz, 1.2 Hz, 2H), 3.91 (s, 3H); LCMS (ESI) m/z 412.1 [C₁₇H₁₅IO₃+18]+.

### Synthesis of methyl 3-benzylbenzofuran-6-carboxylate (J-3)

To a solution of J-2 (850 mg, 2.16 mmol, 1.0 equiv.) in DMA (8.50 mL) at RT under nitrogen atmosphere, was added TBAB (696 mg, 2.16 mmol, 1.0 equiv.), K₃PO₄ (917 mg, 4.32 mmol, 2.0 equiv.) and PdCl₂ (7 mg, 0.04 mmol, 0.02 equiv.). The resultant mixture was stirred at 120° C. for 16 h. Upon the completion of reaction by TLC, the reaction mixture was quenched with H₂O (30 mL) and extracted with EtOAc (3×30 mL). The combined organic layers were washed with brine, dried over Na₂SO₄, filtered, and concentrated. The crude obtained was purified by silica gel (100-200 mesh) column chromatography (elution with 10% EtOAc/hexane) to afford J-3 (500 mg, yield: 87%) as a brown oil. TLC system: EtOAc/hexane (20:80), R_{f} value=0.7; ¹H NMR (400 MHz, CDCl₃) δ ppm: 8.15 (s, 1H), 7.89 (dd, J=8.4 Hz, 1.2 Hz, 1H), 7.51 (s, 1H), 7.43 (d, J=8.4 Hz, 1H), 7.31-7.28 (m, 4H), 7.24-7.23 (m, 1H), 4.04 (s, 2H), 3.93 (s, 3H).

### Synthesis of (3-benzylbenzofuran-6-yl)methanol (J-4)

Compound J-4 was obtained following the ester reduction procedure similar to that for the synthesis of compound A-4, utilizing DIBAL-H (2.0 equiv.) and reaction conditions of 0° C. -RT for 2 h. Compound J-4 was obtained in 80% yield as a yellow gummy liquid following silica gel (100-200 mesh) column chromatography (elution with 10% EtOAc/hexane). TLC system: EtOAc/hexane (20:80), R_{f} value=0.5; ¹H NMR (400 MHz, CDCl₃) δ ppm: 7.49 (s, 1H), 7.41-7.36 (m, 2H), 7.32-7.28 (m, 4H), 7.23-7.20 (m, 1H), 7.18 (d, J=8.0 Hz, 1H), 4.78 (d, J=4.8 Hz, 2H), 4.02 (s, 2H), 1.66 (t, 1H); LCMS (ESI) m/z 221.1 [C₁₆H₁₄O₂−OH]⁺.

### Synthesis of 3-benzylbenzofuran-6-carbaldehyde (J-5)

Compound J-5 was obtained following the alcohol oxidation procedure similar to that for the synthesis of compound A-5, utilizing DMP (2.0 equiv.) and reaction conditions of 0° C.-RT for 2 h. Compound J-5 was obtained in 87% yield as a yellow solid following silica gel (100-200 mesh) column chromatography (elution with 10% EtOAc/hexane). TLC system: EtOAc/hexane (20:80), R_{f} value=0.7; ¹H NMR (400 MHz, CDCl₃) δ ppm: 10.06 (s, 1H), 7.97 (s, 1H), 7.73 (dd, J=8.0 Hz, 0.8 Hz, 1H), 7.58 (s, 1H), 7.52 (d, J=8.0 Hz, 1H), 7.34-7.23 (m, 5H), 4.05 (s, 2H), LCMS (ESI) m/z 236.9 [C₁₆H₁₂O₂+H]⁺.

Synthesis of 2-(((3-benzylbenzofuran-6-yl)methyl)amino)ethan-1-ol as formic acid salt (compound 43): Compound 43 was obtained following the reductive amination procedure similar to that for the synthesis of compound 2, utilizing 2-aminoethan-1-ol (2.0 equiv.), AcOH (0.05 equiv.), NaBH₄ (2.0 equiv.) and reaction conditions of 70° C. for 16 h for imine formation, and 0° C. to RT for 1 h for imine reduction. Compound 43 (formic acid salt) was obtained in 37% yield as an off-white solid following reverse phase column chromatography (Grace column, elution with 17% MeCN/0.1% FA in H₂O). TLC system: MeOH/CH₂Cl₂ (10:90), R_{f} value=0.2; melting point range: 94-97° C.; ¹H NMR (400 MHz, DMSO-d₆) δ ppm: 8.27 (s, 1H; HCOOH), 7.83 (s, 1H), 7.57 (s, 1H), 7.43 (d, J=8.0 Hz, 1H), 7.33-7.27 (m, 4H), 7.21-7.17 (m, 2H), 4.01 (s, 2H), 3.93 (s, 2H), 3.51 (t, J=5.6 Hz, 2H), 2.66 (t, J=5.6 Hz, 2H); LCMS (ESI) m/z 282.2 [C₁₈H₁₉NO₂+H]⁺.

### Example 18: Synthesis of Compound 47 Via Synthesis Route K

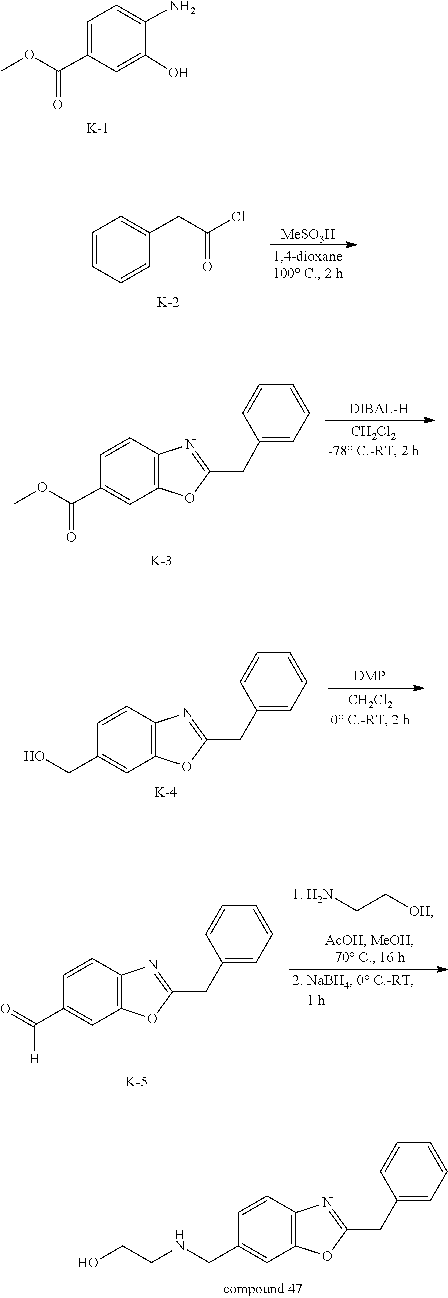

Synthesis of methyl 2-benzylbenzo[d]oxazole-6-carboxylate (K-3): To a stirred solution of methyl 4-amino-3-hydroxybenzoate K-1 (2.50 g, 15.0 mmol, 1.0 equiv.) in 1,4-dioxane (50 mL) at RT, was added 2-phenylacetyl chloride K-2 (2.32 g, 15.0 mmol, 1.0 equiv.) and MeSO₃H (2.92 mL, 45.0 mmol, 3.0 equiv.). The resultant mixture was stirred at 100° C. for 2 h and upon the completion of reaction by TLC, the volatiles were removed and the crude obtained was purified by silica gel (100-200 mesh) column chromatography (elution with 20% EtOAc/hexane) to afford K-3 (400 mg, yield: 10%) as a colourless liquid. TLC system: EtOAc/hexane (20:80), R_{f} value=0.2; ¹H NMR (400 MHz, CDCl₃) δ ppm: 8.16 (s, 1H), 8.04 (dd, J=8.4 Hz, 1.2 Hz, 1H), 7.71 (d, J=8.4 Hz, 1H), 7.40-7.28 (m, 5H), 4.30 (s, 2H), 3.94 (s, 3H); LCMS (ESI) m/z 268.1 [C₁₆H₁₃NO₃+H]⁺.

### Synthesis of (2-benzylbenzo[d]oxazol-6-yl)methanol (K-4)

Compound K-4 was obtained following the ester reduction procedure similar to that for the synthesis of compound A-4, utilizing DIBAL-H (2.5 equiv.) and reaction conditions of −78° C. -RT for 2 h. Compound K-4 was obtained in 84% yield as a light brown gum following silica gel (100-200 mesh) column chromatography (elution with 20% EtOAc/hexane). TLC system: EtOAc/hexane (30:70), R_{f} value=0.3; ¹H NMR (400 MHz, CDCl₃) δ ppm: 7.64 (d, J=8.0 Hz, 1H), 7.51 (s, 1H), 7.39-7.31 (m, 3H), 7.30-7.28 (m, 2H), 7.24-7.22 (m, 1H), 4.79 (s, 2H), 4.27 (s, 2H); LCMS (ESI) m/z 240.1 [C₁₅H₁₃NO₂+H].

### Synthesis of 2-benzylbenzo[d]oxazole-6-carbaldehyde (K-5)

Compound K-5 was obtained following the alcohol oxidation procedure similar to that for the synthesis of compound A-5, utilizing DMP (2.0 equiv.) and reaction conditions of 0° C.-RT for 2 h. Compound K-5 was obtained in 74% yield as a brown gummy liquid following silica gel (100-200 mesh) column chromatography (elution with 10% EtOAc/hexane). TLC system: EtOAc/hexane (30:70), R_{f} value=0.5; ¹H NMR (400 MHz, CDCl₃) δ ppm: 10.06 (s, 1H), 7.99 (s, 1H), 7.87 (dd, J=8.0 Hz, 1.2 Hz, 1H), 7.81 (d, J=8.0 Hz, 1H), 7.41-7.35 (m, 4H), 7.33-7.31 (m, 1H), 4.32 (s, 2H); LCMS (ESI) m/z 238.1 [C₁₅H₁₁NO₂+H]⁺.

### Synthesis of 2-(((2-benzylbenzo[d]oxazol-6-yl)methyl)amino)ethan-1-ol as Formic Acid Salt (Compound 47)

Compound 47 was obtained following the reductive amination procedure similar to that for the synthesis of compound 2, utilizing 2-aminoethan-1-ol (2.0 equiv.), AcOH (0.1 equiv.), NaBH₄ (2.0 equiv.) and reaction conditions of 70° C. for 16 h for imine formation, and 0° C. to RT for 1 h for imine reduction. Compound 47 (formic acid salt) was obtained in 15% yield as a brown gum following reverse phase column chromatography (Grace column, elution with 20% MeCN/0.1% FA in H₂O). TLC system: MeOH/CH₂Cl₂ (10:90), R_{f} value=0.15; ¹H NMR (400 MHz, DMSO-d₆) δ ppm: 8.28 (s, 1H; HCOOH), 7.65 (s, 1H), 7.62 (d, J=8.4 Hz, 1H), 7.37-7.33 (m, 5H), 7.30-7.27 (m, 1H), 4.32 (s, 2H), 3.89 (s, 2H), 3.49 (t, J=5.6 Hz, 2H), 2.61 (t, J=5.6 Hz, 2H); LCMS (ESI) m/z 283.2 [C₁₇H₁₈N₂O₂+H]⁺.

### Example 19: Synthesis of Compound 48 Via Synthesis Route K

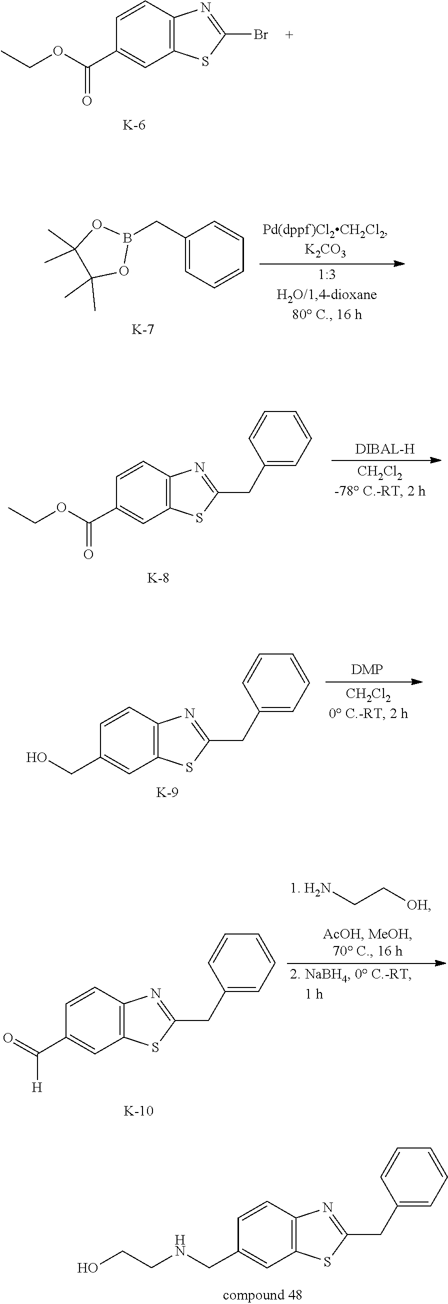

### Synthesis of ethyl 2-benzylbenzo[d]thiazole-6-carboxylate (K-8)

To a stirred solution of ethyl 2-bromobenzo[d]thiazole-6-carboxylate K-6 (1.00 g, 3.49 mmol, 1.0 equiv.) in 3:1 H₂O/1,4-dioxane (20 mL) at RT, was added 2-benzyl-4,4,5,5-tetramethyl-1,3,2-dioxaborolane K-7 (1.52 g, 6.98 mmol, 2.0 equiv.) and K₂CO₃ (2.89 g, 20.9 mmol, 6.0 equiv.). The reaction mixture was degassed under N₂ atmosphere for 10 min before Pd(dppf)Cl₂·CH₂Cl₂ (139 mg, 0.17 mmol, 0.05 equiv.) was added. The resultant mixture was heated and stirred at 80° C. for 16 h. Upon the completion of reaction by TLC, the reaction mixture was poured into H₂O (30 mL) and extracted with EtOAc (3×30 mL). The combined organic layers were washed with brine, dried over Na₂SO₄, concentrated, and purified by silica gel (100-200 mesh) column chromatography (elution with 10% EtOAc/hexane) to afford K-8 (305 mg, semi-pure material) as a colourless gummy liquid. TLC system: EtOAc/hexane (30:70), R_{f} value=0.4; LCMS (ESI) m/z 298.1 [C₁₇H₁₅NO₂S+H]⁺. The semi-pure material was used in the next step without further purification.

### Synthesis of (2-benzylbenzo[d]thiazol-6-yl)methanol (K-9)

Compound K-9 was obtained following the ester reduction procedure similar to that for the synthesis of compound A-4, utilizing DIBAL-H (2.5 equiv.) and reaction conditions of −78° C. -RT for 2 h. Compound K-9 was obtained as a light yellow gummy liquid in its crude. TLC system: EtOAc/hexane (40:60), R_{f} value=0.3; LCMS (ESI) m/z 256.1 [C₁₅H₁₃NOS+H]⁺. The crude material was used in the next step without further purification.

### Synthesis of 2-benzylbenzo[d]thiazole-6-carbaldehyde (K-10)

Compound K-10 was obtained following the alcohol oxidation procedure similar to that for the synthesis of compound A-5, utilizing DMP (1.5 equiv.) and reaction conditions of 0° C.-RT for 2 h. Compound K-10 was obtained in 12% yield over 3 steps as a light yellow gummy liquid following silica gel (100-200 mesh) column chromatography (elution with 15% EtOAc/hexane). TLC system: EtOAc/hexane (30:70), R_{f} value=0.6; ¹H NMR (400 MHz, CDCl₃) δ ppm: 10.08 (s, 1H), 8.33 (d, J=1.2 Hz, 1H), 8.11 (d, J=8.4 Hz, 1H), 7.97 (dd, J=8.4 Hz, 1.6 Hz, 1H), 7.38-7.31 (m, 5H), 4.48 (s, 2H); LCMS (ESI) m/z 254.1 [C₁₅H₁₁NOS+H]+.

### Synthesis of 2-(((2-benzylbenzo[d]thiazol-6-yl)methyl)amino)ethan-1-ol (Compound 48)

Compound 48 was obtained following the reductive amination procedure similar to that for the synthesis of compound 2, utilizing 2-aminoethan-1-ol (1.2 equiv.), AcOH (0.1 equiv.), NaBH₄ (2.5 equiv.) and reaction conditions of 70° C. for 16 h for imine formation, and 0° C. to RT for 1 h for imine reduction. Compound 48 was obtained in 13% yield as a light brown gummy liquid following reverse phase column chromatography (Grace column, elution with 18% MeCN/0.1% formic acid in H₂O). TLC system: MeOH/CH₂Cl₂ (15:85), R_{f} value=0.15; ¹H NMR (400 MHz, DMSO-d₆) δ ppm: 7.98 (s, 1H), 7.90 (d, J=8.4 Hz, 1H), 7.48 (d, J=8.8 Hz, 1H), 7.40-7.34 (m, 4H), 7.30-7.28 (m, 1H), 4.46 (s, 2H), 3.92 (s, 2H), 3.50 (t, J=5.6 Hz, 2H), 2.65 (t, J=5.6 Hz, 2H); LCMS (ESI) m/z 299.1 [C₁₇H₁₈N₂OS+H]⁺.

### Example 20: Synthesis of Compound 50 Via Synthesis Route L

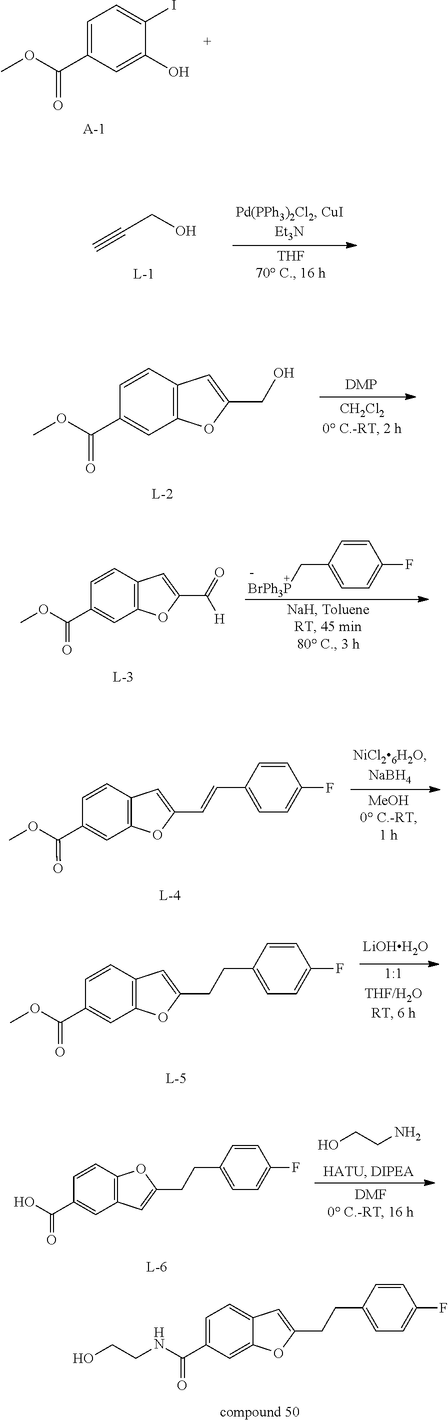

### Synthesis of methyl 2-(hydroxymethyl)benzofuran-6-carboxylate (L-2)

Compound L-2 was obtained following the Pd-catalyzed benzofuran ring formation procedure similar to that for the synthesis of compound A-3, utilizing propargyl alcohol L-1 (3.0 equiv.) and reaction conditions of 70° C. for 16 h. Compound L-2 was obtained in 90% yield as a light yellow gummy solid following silica gel (100-200 mesh) column chromatography (elution with 20% EtOAc/hexane). TLC system: EtOAc/hexane (30:70), R_{f} value=0.2; ¹H NMR (400 MHz, CDCl₃) δ ppm: 8.15 (s, 1H), 7.94 (dd, J=8.0 Hz, 1.6 Hz, 1H), 7.58 (d, J=8.0 Hz, 1H), 6.72 (s, 1H), 4.92 (d, J=6.4 Hz, 2H), 3.95 (s, 3H), 1.98 (t, J=6.4 Hz, 1H); LCMS (ESI) m/z 207.0 [C₁₁H₁₀O₄+H]⁺.

### Synthesis of methyl 2-formylbenzofuran-6-carboxylate (L-3)

Compound L-3 was obtained following the alcohol oxidation procedure similar to that for the synthesis of compound D-6, utilizing DMP (1.5 equiv.) and reaction conditions of 0° C.-RT for 2 h. Compound L-3 was obtained in 72% yield as a light brown solid following silica gel (100-200 mesh) column chromatography (elution with 15% EtOAc/hexane). TLC system: EtOAc/hexane (20:80), R_{f} value=0.4; ¹H NMR (400 MHz, CDCl₃) δ ppm: 9.94 (s, 1H), 8.29 (s, 1H), 8.04 (dd, J=8.0 Hz, 1.2 Hz, 1H), 7.81 (d, J=8.4 Hz, 1H), 7.59 (s, 1H), 3.98 (s, 3H); LCMS (ESI) m/z 205.0 [C₁₁H₈O₄+H]⁺.

### Synthesis of methyl (E/Z)-2-(4-fluorostyryl)benzofuran-6-carboxylate (L-4)

Compound L-4 was obtained following the Wittig reaction procedure similar to that for the synthesis of compound D-9, utilizing (4-fluorobenzyl)triphenylphosphonium bromide (2.5 equiv.) and reaction conditions of 80° C. for 3 h. Compound L-4 was obtained in 40% yield (7:1 ratio of E and Z isomers) as a colourless gum following silica gel (60-120 mesh) column chromatography (elution with 10% EtOAc/hexane). TLC system: EtOAc/hexane (15:85), R_{f} value=0.35; ¹H NMR (400 MHz, CDCl₃) δ ppm: for major trans isomer 8.15 (s, 1H), 7.93 (dd, J=8.0 Hz, 1.2 Hz, 1H), 7.56-7.45 (m, 3H), 7.36 (d, J=16.0 Hz, 1H), 7.11-7.05 (m, 2H), 6.93 (d, J=16.0 Hz, 1H), 6.71 (s, 1H), 3.96 (s, 3H); LCMS (ESI) m/z 297.1 [C₁₈H₁₃FO₃+H]⁺.

### Synthesis of methyl 2-(4-fluorophenethyl)benzofuran-6-carboxylate (L-5)

Compound L-5 was obtained following the alkene reduction procedure similar to that for the synthesis of compound D-10, utilizing NiCl₂·6H₂O (0.1 equiv.) with NaBH₄ (2.5 equiv.) and reaction conditions of 0° C. -RT for 1 h. Compound L-5 was obtained as a colourless gum in its crude. TLC system: EtOAc/hexane (15:85), R_{f} value=0.3; ¹H NMR (400 MHz, CDCl₃) δ ppm: 8.11 (s, 1H), 7.90 (dd, J=8.0 Hz, 2.4 Hz, 1H), 7.49 (d, J=8.0 Hz, 1H), 7.16-7.12 (m, 2H), 6.98-6.94 (m, 1H), 6.39 (s, 2H), 3.94 (s, 3H), 3.11-3.04 (m, 4H); LCMS (ESI) m/z 299.1 [C₁₈H₁₅FO₃+H]⁺. The crude compound was used in the next step without further purification.

### Synthesis of 2-(4-fluorophenethyl)benzofuran-6-carboxylic acid (L-6)

To a stirred solution of L-5 (230 mg, 0.77 mmol, 1.0 equiv.) in 1:1 THF/H₂O (1.0 mL) at RT, was added LiOH·H₂O (96.9 mg, 2.31 mmol, 3.0 equiv.) dissolved in water (0.5 mL). The resultant mixture was stirred at RT for 6 h and upon the completion of reaction by TLC, the reaction mixture was diluted with H₂O (30 mL) and washed with EtOAc (2×20 mL) to remove the non-polar impurities. The resultant aqueous layer was then acidified with 2N HCl (10 mL) and extracted with 10% OMeOH in CH₂Cl₂ (2×50 mL). The combined CH₂Cl₂ extracts were dried over anhydrous Na₂SO₄, filtered, and concentrated under reduced pressure to afford L-6 as a yellow solid (180 mg, yield: 70% over 2 steps). TLC system: MeOH/CH₂Cl₂ (05:95), R_{f} value=0.4; ¹H NMR (400 MHz, CDCl₃) δ ppm: 8.18 (s, 1H), 7.97 (dd, J=8.0 Hz, 1.2 Hz, 1H), 7.52 (d, J=8.0 Hz, 1H), 7.17-7.13 (m, 2H), 6.99-6.95 (m, 1H), 6.41 (s, 2H), 3.12-3.05 (m, 4H); LCMS (ESI) m/z 285.1 [C₁₇H₁₃FO₃+H]⁺.

### Synthesis of 2-(4-fluorophenethyl)-N-(2-hydroxyethyl)benzofuran-6-carboxamide (Compound 50)

To a stirred solution of L-6 (180 mg, 0.63 mmol, 1.0 equiv.) in DMF (3 mL) at 0° C., was added 2-aminoethan-1-ol (58.0 mg, 0.95 mmol, 1.5 equiv.), HATU (361 mg, 0.95 mmol, 1.5 equiv.) and DIPEA (0.3 mL, 1.58 mmol, 2.5 equiv.). The resultant mixture was warmed to RT and stirred at RT for 16 h. Upon the completion of reaction by TLC, the reaction mixture was quenched with ice-cold water (10 mL) and extracted with 10% MeOH in CH₂Cl₂ (2×20 mL). The combined organic layers were dried over anhydrous Na₂SO₄, filtered, and concentrated under reduced pressure. The crude product obtained was purified by reverse phase column chromatography (Grace column, elution with 30% MeCN/0.1% FA in H₂O) to afford compound 50 as a white solid (50 mg, yield: 24%). TLC system: MeOH/CH₂Cl₂ (10:90), R_{f} value=0.3; melting point range: 99-102° C.; ¹H NMR (400 MHz, DMSO-d₆) δ ppm: 8.42 (t, J=5.6 Hz, 1H), 7.99 (s, 1H), 7.73 (dd, J=8.0 Hz, 1.2 Hz, 1H), 7.57 (d, J=8.0 Hz, 1H), 7.31-7.27 (m, 2H), 7.11-7.07 (m, 2H), 6.64 (s, 1H), 4.73 (t, J=5.6 Hz, 1H), 3.52 (q, J=6.0 Hz, 2H), 3.37-3.34 (m, 2H), 3.14-3.10 (m, 2H), 3.05-3.01 (m, 2H); LCMS (ESI) m/z 328.2 [C₁₉H₁₈FNO₃+H]⁺.

### Example 21: Synthesis of Compound 81 Via Synthesis Route A

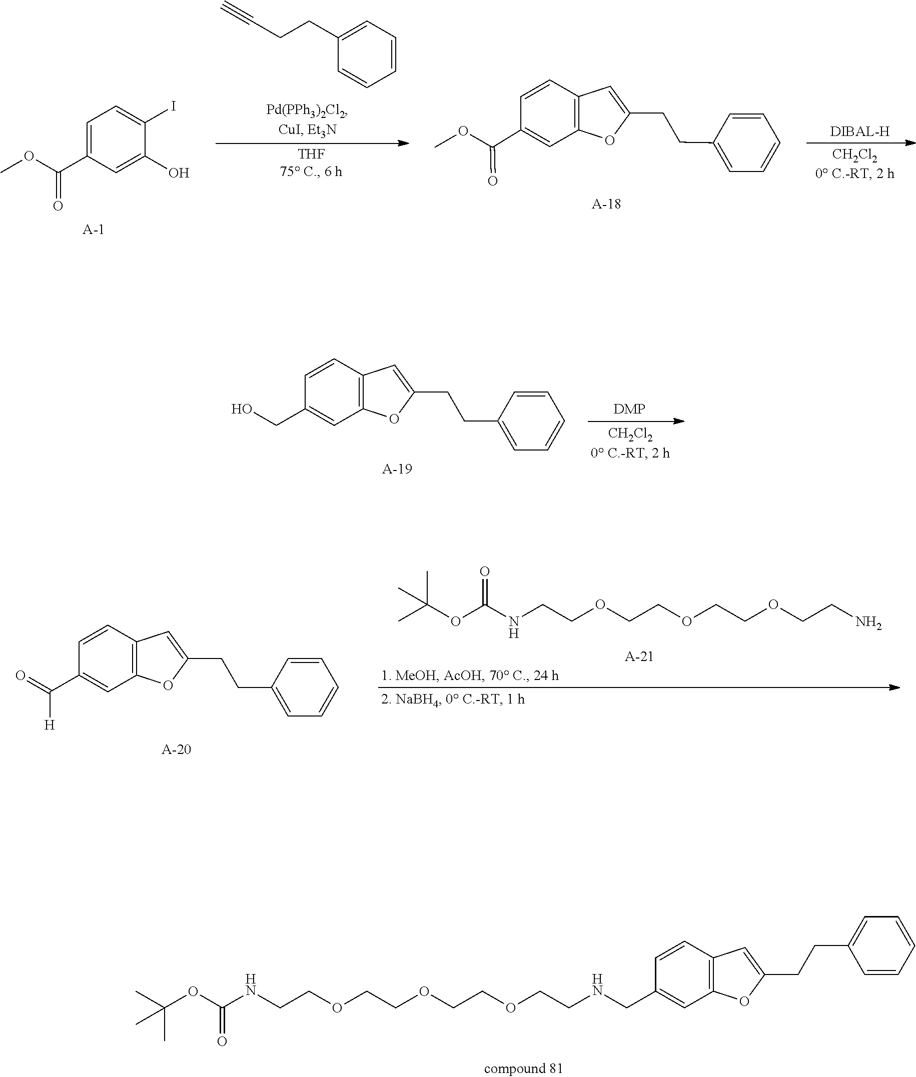

### Synthesis of methyl 2-phenethylbenzofuran-6-carboxylate (A-18)

Compound A-18 was obtained following the Pd-catalyzed benzofuran ring formation procedure similar to that for the synthesis of compound A-3, utilizing but-3-yn-1-ylbenzene (1.0 equiv.) and reaction conditions of 75° C. for 6 h. Compound A-18 was obtained in 51% yield as a yellow liquid following silica gel (60-120 mesh) column chromatography (gradient elution with 0-10% EtOAc/hexane). TLC system: EtOAc/hexane (30:70), R_{f} value=0.6; ¹H NMR (400 MHz, CDCl₃) δ ppm: 8.11 (s, 1H), 7.90 (dd, J=8.0 Hz, 1.6 Hz, 1H), 7.48 (d, J=8.0 Hz, 1H), 7.31-7.28 (m, 2H), 7.23-7.20 (m, 3H), 6.40 (s, 1H), 3.94 (s, 3H), 3.12-3.09 (m, 4H); LCMS (ESI) m/z 281.1 [C₁₈H₁₆O₃+H]⁺.

### Synthesis of (2-phenethylbenzofuran-6-yl)methanol (A-19)

Compound A-19 was obtained following the ester reduction procedure similar to that for the synthesis of compound A-4, utilizing DIBAL-H (3.0 equiv.) and reaction conditions of 0° C. -RT for 2 h. Compound A-19 was obtained in 89% yield as a white solid following silica gel (60-120 mesh) column chromatography (gradient elution with 15-20% EtOAc/hexane). TLC system: EtOAc/hexane (20:80), R_{f} value=0.2; ¹H NMR (400 MHz, CDCl₃) δ ppm: 7.45-7.43 (m, 2H), 7.31-7.27 (m, 2H), 7.22-7.17 (m, 4H), 6.34 (s, 1H), 4.78 (s, 2H), 3.07 (s, 4H), 1.66 (br. s, 1H).

### Synthesis of 2-phenethylbenzofuran-6-carbaldehyde (A-20)

Compound A-20 was obtained following the alcohol oxidation procedure similar to that for the synthesis of compound A-5, utilizing DMP (1.5 equiv.) and reaction conditions of 0° C.-RT for 1 h. Compound A-20 was obtained in 64% yield as a yellow solid following silica gel (60-120 mesh) column chromatography (gradient elution with 8-10% EtOAc/hexane). TLC system: EtOAc/hexane (20:80), R_{f} value=0.4; ¹H NMR (400 MHz, CDCl₃) δ ppm: 10.1 (s, 1H), 7.93 (s, 1H), 7.74 (dd, J=8.0, 1.2 Hz, 1H), 7.57 (d, J=8.0 Hz, 1H), 7.32-7.28 (m, 2H), 7.23-7.20 (m, 3H), 6.45 (s, 1H), 3.17-3.07 (m, 4H); LCMS (ESI) m/z 251.1 [C₁₇H₁₄O₂+H]⁺.

### Synthesis of tert-butyl (1-(2-phenethylbenzofuran-6-yl)-5,8,11-trioxa-2-azatridecan-13-yl)carbamate (Compound 81)

Compound 81 was obtained following the reductive amination procedure similar to that for the synthesis of compound 2, utilizing tert-butyl (2-(2-(2-(2-aminoethoxy)ethoxy)ethoxy)ethyl)carbamate A-21 (0.8 equiv.), AcOH (0.2 equiv.), NaBH₄ (2.5 equiv.) and reaction conditions of 70° C. for 24 h for imine formation, and 0° C. to RT for 1 h for imine reduction. Compound 81 was obtained in 9% yield as a colourless gummy liquid following Prep HPLC [Prep conditions: Sunfire C18 column, 5 μm, 19×250 mm; Buffer A: 0.1% formic acid in water; Buffer B: MeCN; Gradient (Time/% B): 0.1/5, 15/60, 25/95; Flow Rate: 13 mL/min] purification. TLC system: MeOH/CH₂Cl₂ (10:90), R_{f} value=0.3; ¹H NMR (400 MHz, DMSO-d₆) δ ppm: 7.46-7.42 (m, 2H), 7.28-7.25 (m, 4H), 7.20-7.14 (m, 2H), 6.78 (t, 1H), 6.52 (s, 1H), 3.82 (s, 2H), 3.50-3.45 (m, 10H), 3.35-3.34 (m, 2H), 3.10-2.99 (m, 6H), 2.68-2.67 (m, 2H), 1.36 (s, 9H); LCMS (ESI) m/z 527.4 [C₃₀H₄₂N₂O₆+H]⁺.

### Example 22: Synthesis of Compound 82 Via Synthesis Route A

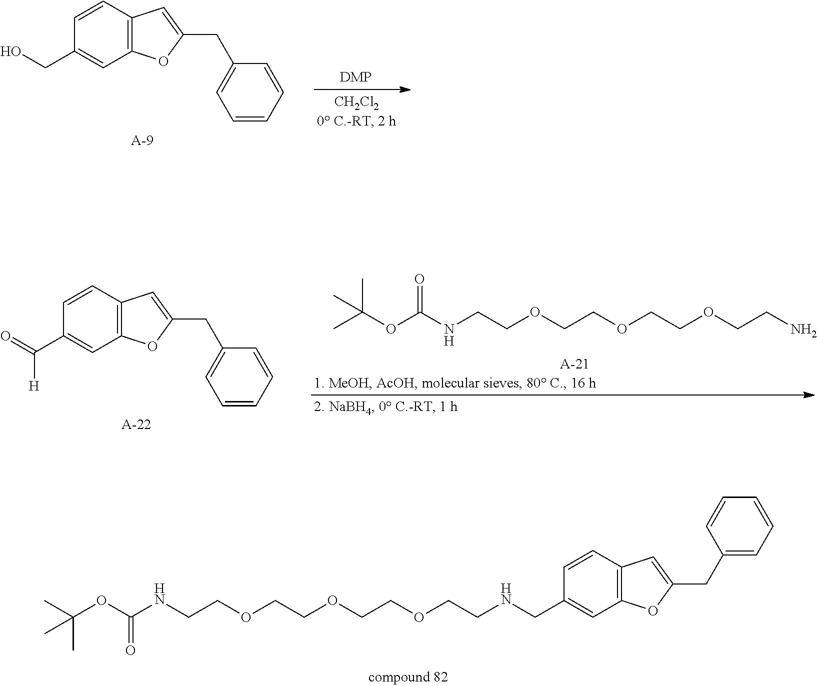

### Synthesis of 2-benzylbenzofuran-6-carbaldehyde (A-22)

Compound A-22 was obtained following the alcohol oxidation procedure similar to that for the synthesis of compound A-5, utilizing DMP (1.5 equiv.) and reaction conditions of 0° C.-RT for 2 h. Compound A-22 was obtained in 83% yield as a yellow liquid following silica gel (60-120 mesh) column chromatography (gradient elution with 3-5% EtOAc/hexane). TLC system: EtOAc/hexane (20:80), R_{f} value=0.6; ¹H NMR (400 MHz, CDCl₃) δ ppm: 10.0 (s, 1H), 7.91 (s, 1H), 7.73 (dd, J=8.0, 1.2 Hz, 1H), 7.57 (d, J=8.0 Hz, 1H), 7.37-7.27 (m, 5H), 6.44 (d, J=1.6 Hz, 1H), 4.15 (s, 2H).

### Synthesis of tert-butyl (1-(2-benzylbenzofuran-6-yl)-5,8,11-trioxa-2-azatridecan-13-yl)carbamate (Compound 82)

Compound 82 was obtained following the reductive amination procedure similar to that for the synthesis of compound 2, utilizing tert-butyl (2-(2-(2-(2-aminoethoxy)ethoxy)ethoxy)ethyl)carbamate A-21 (1.2 equiv.), 4 Å molecular sieves, AcOH (0.3 equiv.), NaBH₄ (2.0 equiv.) and reaction conditions of 80° C. for 16 h for imine formation, and 0° C. to RT for 2 h for imine reduction. Compound 82 was obtained in 25% yield as a colourless gummy liquid following Prep HPLC [Prep conditions: X-Terra C18 column, 5 μm, 21.2×150 mm; Buffer A: 0.1% liquid NH₃ in H₂O; Buffer B: MeCN; Gradient (Time/% B): 0/45, 16/65, 16.1/98; Flow Rate: 12 mL/min] purification. TLC system: MeOH/CH₂Cl₂ (10:90), R_{f} value=0.1; ¹H NMR (400 MHz, DMSO-d₆) δ ppm: 7.45 (d, J=8.0 Hz, 1H), 7.41 (s, 1H), 7.35-7.30 (m, 4H), 7.27-7.23 (m, 1H), 7.14 (dd, J=8.0 Hz, 1.2 Hz, 1H), 6.77 (t, J=5.2 Hz, 1H), 6.55 (d, J=0.8 Hz, 1H), 4.12 (s, 2H), 3.77 (s, 2H), 3.49-3.44 (m, 10H), 3.36-3.34 (m, 2H), 3.04 (q, J=5.6 Hz, 2H), 2.61 (t, J=5.6 Hz, 2H), 1.36 (s, 9H); LCMS (ESI) m/z 513.4 [C₂₉H₄₀N₂O₆+H]⁺.

### Example 23: Synthesis of Compound 83 Via Synthesis Route A

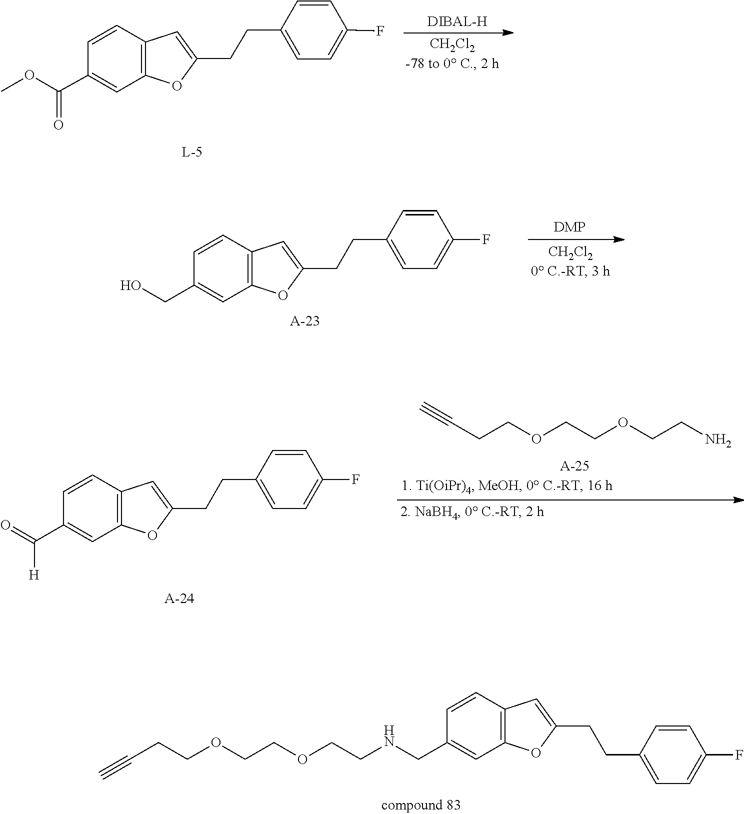

### Synthesis of (2-(4-fluorophenethyl)benzofuran-6-yl)methanol (A-23)

Compound A-23 was obtained following the ester reduction procedure similar to that for the synthesis of compound A-4, utilizing DIBAL-H (2.0 equiv.) and reaction conditions of −78° C. to 0° C. for 2 h. Compound A-23 was obtained in 66% yield as a pale-yellow solid following silica gel (100-200 mesh) column chromatography (gradient elution with 0-30% EtOAc/hexane). TLC system: EtOAc/hexane (20:80), R_{f} value=0.4; ¹H NMR (400 MHz, CDCl₃) δ ppm: 7.45-7.43 (m, 2H), 7.19 (dd, J=8.0 Hz, 1.6 Hz, 1H), 7.16-7.12 (m, 2H), 6.98-6.94 (m, 2H), 6.32 (s, 1H), 4.78 (s, 2H), 3.05 (s, 4H); LCMS (ESI) m/z 253.1 [C₁₇H₁₅FO₂−OH]⁺.

### Synthesis of 2-(4-fluorophenethyl)benzofuran-6-carbaldehyde (A-24)

Compound A-24 was obtained following the alcohol oxidation procedure similar to that for the synthesis of compound A-5, utilizing DMP (2.0 equiv.) and reaction conditions of 0° C.-RT for 3 h. Compound A-24 was obtained in 60% yield as a yellow solid following silica gel (100-200 mesh) column chromatography (gradient elution with 0-20% EtOAc/hexane). TLC system: EtOAc/hexanes (20:80), R_{f} value=0.6; ¹H NMR (400 MHz, CDCl₃) δ ppm: 10.05 (s, 1H), 7.93 (s, 1H), 7.74 (dd, J=8.0 Hz, 1.2 Hz, 1H), 7.58 (d, J=8.0 Hz, 1H), 7.17-7.13 (m, 2H), 6.99-6.94 (m, 2H), 6.42 (s, 1H), 3.14-3.04 (m, 4H); LCMS (ESI) m/z 269.1 [C₁₇H₁₃FO₂+H]+.

### Synthesis of 2-(2-(but-3-yn-1-yloxy)ethoxy)-N-((2-(4-fluorophenethyl)benzofuran-6-yl)methyl)ethan-1-amine (Compound 83)

To a stirred solution of A-24 (500 mg, 1.86 mmol, 1.0 equiv.) in MeOH (3 mL) at 0° C., was added 2-(2-(but-3-yn-1-yloxy)ethoxy)ethan-1-amine A-25 (527 mg, 3.35 mmol, 1.8 equiv.) followed by Ti(OiPr)₄ (0.5 mL). The reaction mixture was warmed to RT and stirred at RT for 16 h before it was cooled to 0° C. and NaBH₄ (141 mg, 3.72 mmol, 2.0 equiv.) was added. The resultant mixture was warmed to RT and stirred at RT for 2 h. Upon the completion of reaction by TLC, ice-cold water (30 mL) was added to the reaction mixture and the mixture was extracted with 10% MeOH in CH₂Cl₂ (3×30 mL). The combined organic layers were dried over anhydrous Na₂SO₄, filtered, and concentrated under reduced pressure. The crude compound obtained was purified by silica gel (60-120 mesh) column chromatography (elution with 10% MeOH/CH₂Cl₂) to afford compound 83 (650 mg, yield: 85%) as a colourless gum. TLC system: MeOH/CH₂Cl₂ (10:90), R_{f} value=0.4; ¹H NMR (400 MHz, DMSO-d₆) δ ppm: 7.43-7.41 (m, 2H), 7.31-7.27 (m, 2H), 7.15-7.07 (m, 3H), 6.51 (s, 1H), 3.78 (s, 2H), 3.53-3.46 (m, 8H), 3.08-2.99 (m, 4H), 2.78 (t, J=2.4 Hz, 1H), 2.63 (t, J=6.0 Hz, 2H), 2.39-2.37 (m, 2H), 2.12 (br. s, 1H); LCMS (ESI) m/z 410.2 [C₂₅H₂₈FNO₃+H]⁺.

### Example 24: Synthesis of Compound 95 Via Synthesis Route A

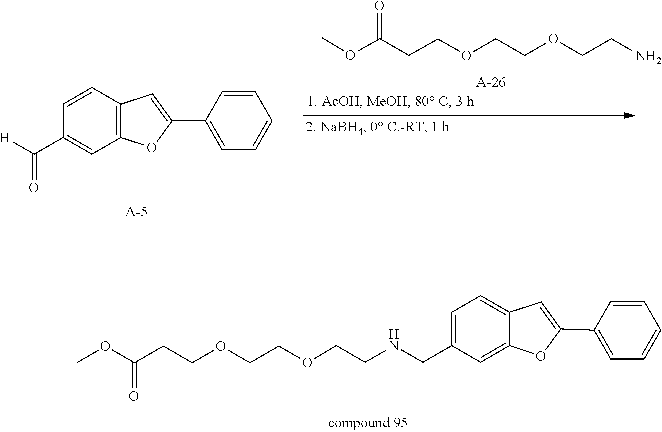

### Synthesis of methyl 3-(2-(2-(((2-phenylbenzofuran-6-yl)methyl)amino)ethoxy)ethoxy)propanoate (Compound 95)

To a stirred solution of 2-phenylbenzofuran-6-carbaldehyde A-5 (400 mg, 1.80 mmol, 1.0 equiv.) and methyl 3-(2-(2-aminoethoxy)ethoxy)propanoate A-26 (413 mg, 2.16 mmol, 1.2 equiv.) in MeOH (20 mL) at RT, was added AcOH (33 mg, 0.54 mmol, 0.3 equiv.). The reaction mixture was stirred at 80° C. for 3 h before it was cooled to 0° C., and NaBH₄ (136 mg, 3.60 mmol, 2.0 equiv.) was added. The resultant mixture was stirred at RT for 1 h. Upon the completion of reaction by TLC, the reaction mixture was quenched with ice-cold water (20 mL) and extracted with CH₂Cl₂ (2×40 mL). The combined organic layer was dried over anhydrous Na₂SO₄, filtered, and concentrated under reduced pressure. The crude material obtained was purified by Prep HPLC [Prep conditions: X-Terra C18 column, 5 μm, 19×150 mm; Buffer A: 0.1% aq. NH₃ in 10 mM ammonium bicarbonate in H₂O; Buffer B: MeCN; gradient (Time/% B): 0/30, 14/65, 15.1/98; Flow rate: 14 mL/min] to afford compound 95 (44 mg, yield: 6%) as a colourless liquid. TLC system: MeOH/CH₂Cl₂ (5:95), R_{f} value=0.2; ¹H NMR (400 MHz, DMSO-d₆) δ ppm: 7.92-7.89 (m, 2H), 7.58-7.56 (m, 2H), 7.51 (t, J=7.6 Hz, 2H), 7.42-7.38 (m, 2H), 7.23 (d, J=8.8 Hz, 1H), 3.83 (s, 2H), 3.62 (t, J=6.4 Hz, 2H), 3.57 (s, 3H), 3.50-3.46 (m, 6H), 2.65 (t, J=5.6 Hz, 2H), 2.54-2.49 (m, 2H), 2.22 (br. s, 1H); LCMS (ESI) m/z 398.1 [C₂₃H₂₇NO₅+H]⁺.

### Example 25: Synthesis of Compound 98 Via Synthesis Route B

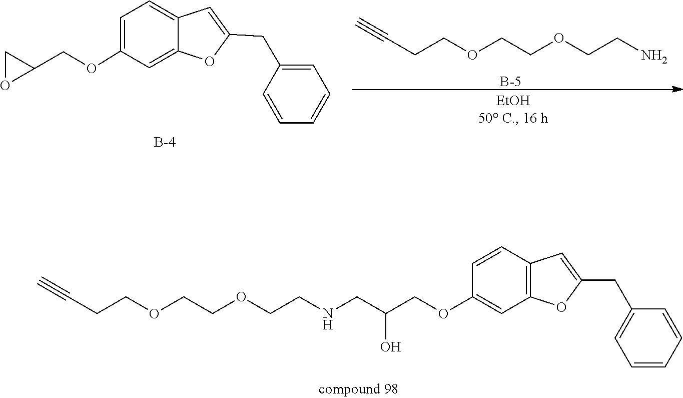

### Synthesis of 1-((2-benzylbenzofuran-6-yl)oxy)-3-((2-(2-(but-3-yn-1-yloxy)ethoxy)ethyl)amino)propan-2-ol (Compound 98)

To a stirred solution of 2-benzyl-6-(oxiran-2-ylmethoxy)benzofuran B-4 (250 mg, 0.89 mmol, 1.0 equiv.) in EtOH (4 mL) at RT, was added 2-(2-(but-3-yn-1-yloxy)ethoxy)ethan-1-amine B-5 (210 mg, 1.34 mmol, 1.5 equiv.). The reaction mixture was stirred at 50° C. for 16 h. Upon the completion of reaction by TLC, the reaction mixture was concentrated under reduced pressure and the crude material obtained was purified by Prep HPLC [Prep conditions: Shimpack C18 column, 5 μm, 21.5×250 mm; Buffer A: 0.1% aq. NH₃ in 10 mM ammonium bicarbonate in H₂O; Buffer B: MeCN/H₂O (80:20); Gradient (Time/% B): 0/60, 15/90, 18/95, 19/98; Flow Rate: 14 mL/min] to afford compound 98 (29 mg, yield: 7%) as a white solid. TLC system: MeOH/CH₂Cl₂ (10:90), R_{f} value=0.3; ¹H NMR (400 MHz, DMSO-d₆) δ ppm: 7.39 (d, J=8.4 Hz, 1H), 7.35-7.29 (m, 4H), 7.26-7.22 (m, 1H), 7.09 (d, J=2.0 Hz, 1H), 6.81 (dd, J=8.4 Hz, 2.0 Hz, 1H), 6.50 (s, 1H), 4.98 (d, J=4.0 Hz, 1H), 4.09 (s, 2H), 3.94-3.91 (m, 1H), 3.88-3.85 (m, 2H), 3.53-3.44 (m, 8H), 2.78 (t, J=2.8 Hz, 1H), 2.68-2.65 (m, 2H), 2.59-2.55 (m, 2H), 2.39-2.33 (m, 2H), 1.8 (br. s, 1H); LCMS (ESI) m/z 438.2 [C₂₆H₃₁NO₅+H]⁺.

### Example 26: Synthesis of Compound 100 Via Synthesis Route M

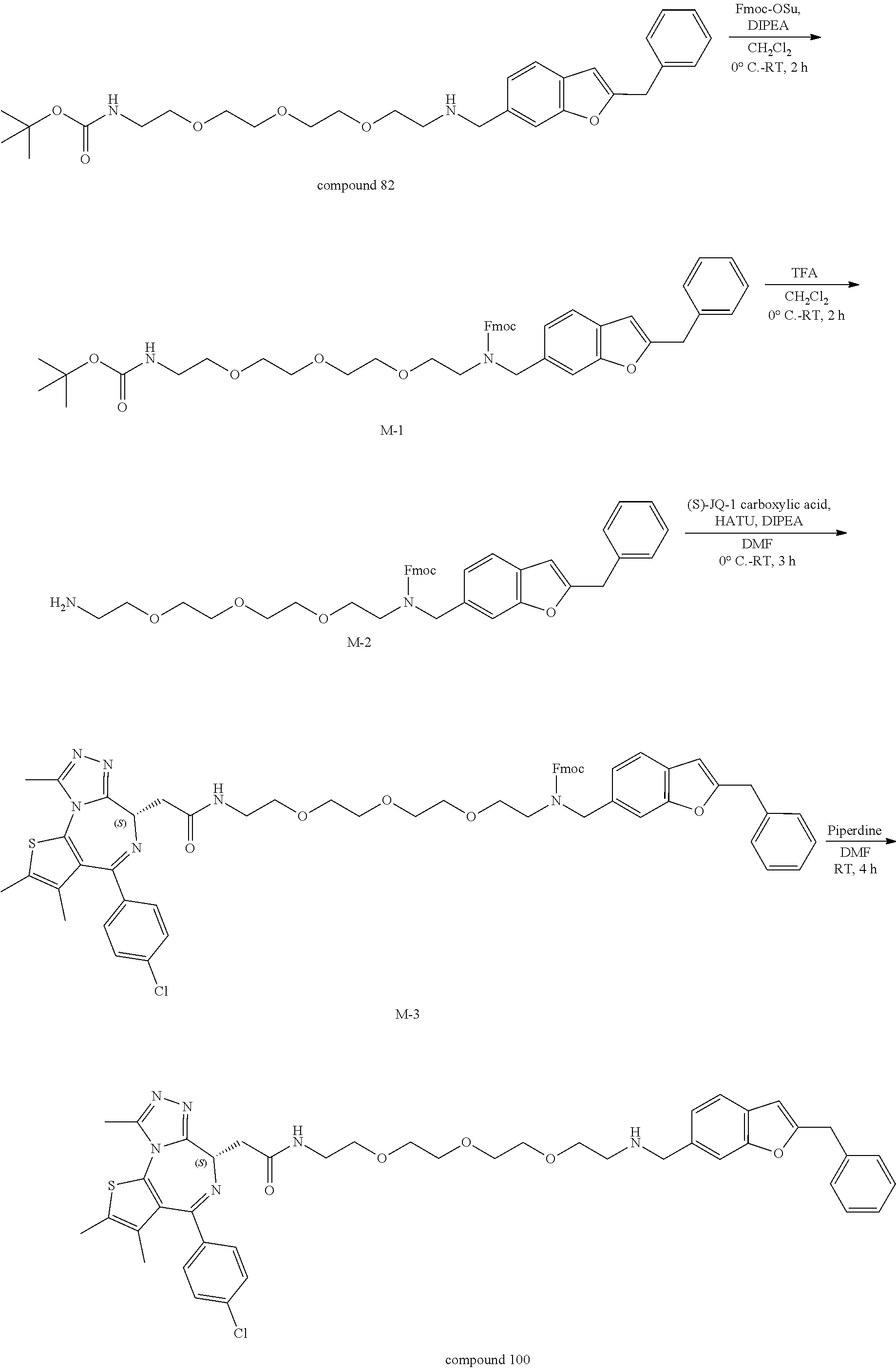

### Synthesis of (9H-fluoren-9-yl)methyl ((2-benzylbenzofuran-6-yl)methyl)(2,2-dimethyl-4-oxo-3,8,11,14-tetraoxa-5-azahexadecan-16-yl)carbamate (M-1)

To a stirred solution of compound 82 (500 mg, 0.98 mmol, 1 equiv.) in CH₂Cl₂ (5 mL) at 0° C., was added DIPEA (0.34 mL, 1.96 mmol, 2 equiv.) and Fmoc-OSu (398 mg, 1.18 mmol, 1.2 equiv.). The resultant mixture was warmed to RT and stirred at RT for 2 h. Upon the completion of reaction by TLC, the reaction mixture was quenched with ice-cold water (10 mL) and extracted with CH₂Cl₂ (2×30 mL). The combined organic layers were dried over anhydrous Na₂SO₄, filtered, concentrated under reduced pressure and purified by silica gel (60-120 mesh) column chromatography (gradient elution with 30-35% EtOAc/hexane) to afford M-1 as a light green gummy liquid (400 mg, yield: 56%). TLC system: EtOAc/hexane (30:70), R_{f} value=0.2; ¹H NMR (400 MHz, CDCl₃) δ ppm: 7.75 (d, J=7.6 Hz, 1H), 7.67 (d, J=7.2 Hz, 1H), 7.59 (d, J=6.8 Hz, 1H), 7.43-7.28 (m, 10H), 7.32-7.26 (m, 1H), 7.17-7.11 (m, 2H), 7.01-6.88 (m, 1H), 6.35 (d, J=12.0 Hz, 1H), 5.00 (br. s, 1H), 4.60-4.49 (m, 4H), 4.26-4.19 (m, 1H), 3.58-3.41 (m, 13H), 3.29-3.17 (m, 4H), 1.42 (s, 9H); LCMS (ESI) m/z 735.4 [C₄₄H₅₀N₂O₈+H]⁺.

### Synthesis of (9H-fluoren-9-yl)methyl (2-(2-(2-(2-aminoethoxy)ethoxy)ethoxy)ethyl)((2-benzylbenzofuran-6-yl)methyl)carbamate (M-2)

To a stirred solution of M-1 (400 mg, 0.54 mmol, 1 equiv.) in CH₂Cl₂ (4 mL) at 0° C., was added TFA (0.4 mL). The reaction mixture was warmed to RT and stirred at RT for 2 h. Upon the completion of reaction by TLC, the volatiles were removed under reduced pressure and the residue was triturated with n-pentane (2×10 mL) to afford M-2 (TFA salt) as a pink gummy liquid (450 mg, crude). TLC system: MeOH/CH₂Cl₂ (10:90), R_{f} value=0.1; LCMS (ESI) m/z 635.4 [C₃₉H₄₂N₂O₆+H]⁺. The crude compound was used in the next step without further purification.

### Synthesis of (9H-fluoren-9-yl)methyl (S)-((2-benzylbenzofuran-6-yl)methyl)(1-(4-(4-chlorophenyl)-2,3,9-trimethyl-6H-thieno[3,2-f][1,2,4]triazolo[4,3-a][1,4]diazepin-6-yl)-2-oxo-6,9,12-trioxa-3-azatetradecan-14-yl)carbamate (M-3)

To a stirred solution of M-2 (230 mg, 0.36 mmol, 1 equiv.) in DMF (5 mL) at 0° C., was added (S)-2-(4-(4-chlorophenyl)-2,3,9-trimethyl-6H-thieno[3,2-f][1,2,4]triazolo[4,3-a][1,4]diazepin-6-yl)acetic acid (130 mg, 0.32 mmol, 0.9 equiv.), HATU (205 mg, 0.54 mmol, 1.5 equiv.) and DIPEA (0.19 mL, 1.08 mmol, 3.0 equiv.). The resultant mixture was warmed to RT and stirred at RT for 3 h. Upon the completion of reaction by TLC, the reaction mixture was quenched with ice-cold water (10 mL) and extracted with CH₂Cl₂ (2×30 mL). The combined organic layers were dried over anhydrous Na₂SO₄, filtered, and concentrated under reduced pressure. The crude product obtained was purified by reverse phase column chromatography (Grace column, gradient elution with 30-35% MeCN/0.1% formic acid in H₂O) to afford M-3 as a pale yellow solid (190 mg, yield: 51%). TLC system: MeOH/CH₂Cl₂ (10:90), R_{f} value=0.3; ¹H NMR (400 MHz, CDCl₃) δ ppm: 7.74 (d, J=7.2 Hz, 1H), 7.67 (d, J=7.6 Hz, 1H), 7.58 (d, J=7.2 Hz, 1H), 7.42-7.29 (m, 14H), 7.15-7.10 (m, 2H), 7.10-6.85 (m, 2H), 6.35 (d, J=8.0 Hz, 1H), 4.64 (t, J=5.2 Hz, 1H), 4.59-4.48 (m, 3H), 4.26-4.18 (m, 1H), 4.09 (d, J=5.2 Hz, 2H), 3.56-3.18 (m, 18H), 2.64 (s, 3H), 2.39 (s, 3H), 1.66 (s, 3H); LCMS (ESI) m/z 1017.4 [C₅₈H₅₇C₁N₆O₇S+H]⁺.

### Synthesis of (S)-N-(1-(2-benzylbenzofuran-6-yl)-5,8,11-trioxa-2-azatridecan-13-yl)-2-(4-(4-chlorophenyl)-2,3,9-trimethyl-6H-thieno[3,2-f][1,2,4]triazolo[4,3-a][1,4]diazepin-6-yl)acetamide (Compound 100)

To a stirred solution of M-3 (190 mg, 0.19 mmol, 1 equiv.) in DMF (4 mL) at 0° C., was added piperidine (0.19 mL). The resultant mixture was warmed to RT and stirred at RT for 4 h. Upon the completion of reaction by TLC, the reaction mixture was quenched with ice-cold water (10 mL) and extracted with 10% MeOH/CH₂Cl₂ (2×30 mL). The combined organic layers were dried over anhydrous Na₂SO₄, filtered, and concentrated under reduced pressure. The crude product obtained was purified by reverse phase column chromatography (Grace column, gradient elution with 30-35% MeCN/0.1% formic acid in H₂O) to afford compound 100 as an off-white solid (50 mg, yield: 33%). TLC system: MeOH/CH₂Cl₂ (10:90), R_{f} value=0.1; ¹H NMR (400 MHz, DMSO-d₆) δ ppm: 8.27 (t, J=5.6 Hz, 1H), 7.49-7.45 (m, 4H), 7.43-7.40 (m, 2H), 7.35-7.29 (m, 4H), 7.26-7.22 (m, 1H), 7.17 (dd, J=8.0 Hz, 1.2 Hz, 1H), 6.57 (s, 1H), 4.52-4.49 (m, 1H), 4.13 (s, 2H), 3.86 (s, 2H), 3.52-3.42 (m, 12H), 3.30-3.19 (m, 4H), 2.70 (t, J=5.6 Hz, 2H), 2.59 (s, 3H), 2.40 (s, 3H), 1.61 (s, 3H); LCMS (ESI) m/z 795.4 [C₄₃H₄₇C₁N₆O₅S+H]⁺.

### Example 27: Synthesis of Compound 104 Via Synthesis Route M

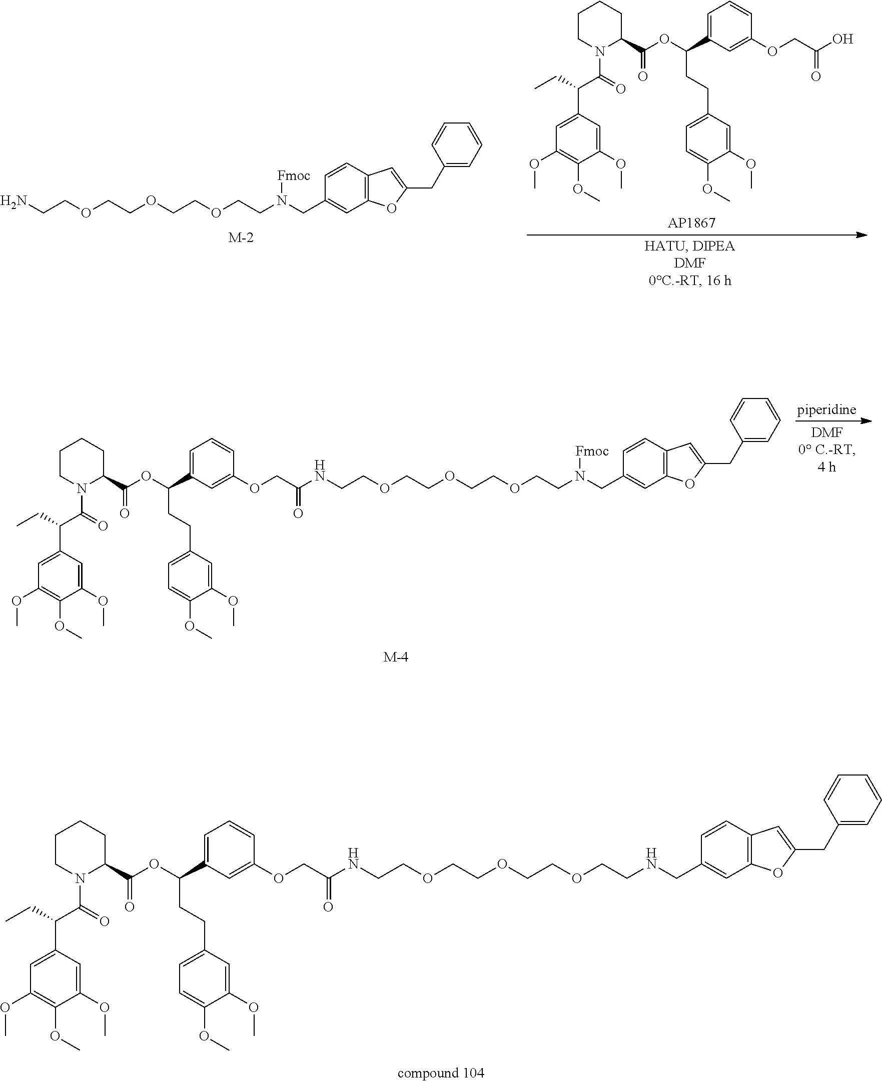

### Synthesis of (R)-1-(3-((4-((2-benzylbenzofuran-6-yl)methyl)-1-(9H-fluoren-9-yl)-3,17-dioxo-2,7,10,13-tetraoxa-4,16-diazaoctadecan-18-yl)oxy)phenyl)-3-(3,4-dimethoxyphenyl)propyl (S)-1-((S)-2-(3,4,5-trimethoxyphenyl)butanoyl)piperidine-2-carboxylate (M-4)

To a stirred solution of AP1867 (80 mg, 0.12 mmol, 1.0 equiv.) in DMF (2 mL) at 0° C., was added M-2 (114 mg, 0.18 mmol, 1.5 equiv.), HATU (60.8 mg, 0.16 mmol, 1.3 equiv.) and DIPEA (0.05 mL, 0.30 mmol, 2.5 equiv.). The resultant mixture was warmed to RT and stirred at RT for 16 h. Upon the completion of reaction by TLC, the reaction mixture was quenched with ice-cold water (10 mL) and extracted with EtOAc (2×25 mL). The combined organic layers were dried over anhydrous Na₂SO₄, filtered, and concentrated under reduced pressure. The crude compound obtained was purified by silica gel (100-200 mesh) column chromatography (eluted with EtOAc and 2% MeOH/CH₂Cl₂) to afford M-4 as a gummy liquid (110 mg, yield: 70%). TLC system: EtOAc, R_{f} value=0.6; LCMS (ESI) m/z 1310.9 [C₇₇H₈₇N₃O₁₆+H]⁺.

### Synthesis of (R)-1-(3-((1-(2-benzylbenzofuran-6-yl)-15-oxo-5,8,11-trioxa-2,14-diazahexadecan-16-yl)oxy)phenyl)-3-(3,4-dimethoxyphenyl)propyl (S)-1-((S)-2-(3,4,5-trimethoxyphenyl)butanoyl)piperidine-2-carboxylate (Compound 104)

To a stirred solution of M-4 (110 mg, 0.084 mmol, 1.0 equiv.) in DMF (2.0 mL) at 0° C., was added piperidine (17 μL, 0.17 mmol, 2.0 equiv.). The reaction mixture was warmed to RT and stirred at RT for 4 h. Upon the completion of reaction by TLC, the reaction mixture was concentrated under reduced pressure and purified by prep-HPLC [Prep conditions: Luna C18 column, 5 μm, 19.1×250 mm; Buffer A: 0.1% formic acid in water, Buffer B: MeCN; Gradient (Time/% B): 0/25, 10/50, 20/60, 25/95; Flow Rate: 13 mL/min] to afford compound 104 as an off-white semi-solid (17.5 mg, yield: 16%). TLC system: MeOH/CH₂Cl₂ (10:90), R_{f} value=0.2; ¹H NMR (400 MHz, DMSO-d₆) δ ppm: 8.05 (t, J=5.6 Hz, 1H), 7.50-7.48 (m, 2H), 7.34-7.15 (m, 7H), 6.84-6.81 (m, 3H), 6.75-6.72 (m, 1H), 6.64-6.58 (m, 3H), 6.52 (s, 2H), 5.54-5.51 (m, 1H), 5.28-5.26 (m, 1H), 4.48-4.46 (m, 2H), 4.13 (s, 2H), 4.02-3.82 (m, 4H), 3.74-3.70 (m, 8H), 3.58-3.56 (m, 8H), 3.49-3.47 (m, 10H), 3.41 (t, J=6.0 Hz, 2H), 3.28-3.26 (m, 2H), 2.75 (br. s, 2H), 2.64-2.58 (m, 1H), 2.38-2.34 (m, 1H), 2.19-2.15 (m, 1H), 1.95-1.87 (m, 3H), 1.64-1.54 (m, 4H), 1.39-1.36 (m, 1H), 0.80 (t, J=7.2 Hz, 3H); LCMS (ESI) m/z 1088.8 [C₆₂H₇₇N₃O₁₄+H]⁺.

Where absolute stereochemistry has not been indicated, compounds herein are racemic mixtures with relative stereochemistry as drawn.

TABLE 4
Summary of compounds (series I) prepared according to the Examples above
and characterizations thereof

| Cpd | Synthesis | | NMR and Mass |
|---|---|---|---|
| No. | Route | Structure | Spectrometry |
| 1 | A | 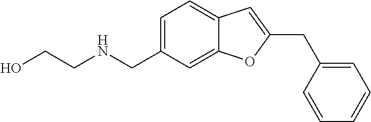 [SMILES: OCCNCc1ccc2cc(Cc3ccccc3)oc2c1] | ¹H NMR (400 MHz, DMSO-d₆) δ (ppm): 8.23 (s, 1H; HCOOH), 7.50-7.47 (m, 2H), 7.35-7.30 (m, 4H), 7.27-7.23 (m, 1H), 7.20- 7.18 (m, 1H), 6.58 (s, 1H), 4.14 (s, 2H), 3.88 (s, 2H), 3.49 (t, J = 5.6 Hz, 2H), 2.63 |
| | | | (t, J = 5.6 Hz, 2H). |
| | | | MS (ESI) m/z 282.1 |
| | | | [C₁₈H₁₉NO₂ + H]⁺. |
| 2 | A | 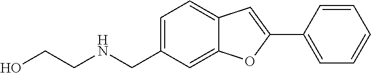 [SMILES: OCCNCc1ccc2cc(-c3ccccc3)oc2c1] | ¹H NMR (400 MHz, DMSO-d₆) δ (ppm): 8.31 (br. s, 1H; HCOOH), 7.93- 7.90 (m, 2H), 7.62 (s, 1H), 7.59 (d, J = 8.0 Hz, 1H), |
| | | | 7.53-7.49 (m, 2H), 7.43- |
| | | | 7.38 (m, 2H), 7.26 (dd, J = |
| | | | 8.0 Hz, 1.2 Hz, 1H), 3.89 (s, |
| | | | 2H), 3.50 (t, J = 6.0 Hz, 2H), |
| | | | 2.63 (t, J = 6.0 Hz, 2H). |
| | | | MS (ESI) m/z 268.1 |
| | | | [C₁₇H₁₇NO₂ + H]⁺. |
| 3 | A | 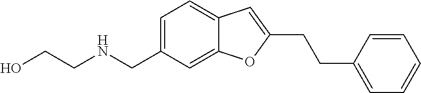 [SMILES: OCCNCc1ccc2cc(CCc3ccccc3)oc2c1] | ¹H NMR (400 MHz, DMSO-d₆) δ (ppm): 8.34 (br. s, 1H; HCOOH), 7.53 (br. s, 1H), 7.46 (d, J = 7.6 Hz, 1H), 7.30-7.25 (m, 4H), 7.21-7.17 (m, 2H), 6.55 (s, |
| | | | 1H), 3.92 (s, 2H), 3.52 (t, J = |
| | | | 5.6 Hz, 2H), 3.10-3.07 |
| | | | (m, 2H), 3.04-2.99 (m, 2H), |
| | | | 2.67 (t, J = 5.6 Hz, 2H). |
| | | | MS (ESI) m/z 296.1 |
| | | | [C₁₉H₂₁NO₂ + H]⁺. |
| 4 | A | 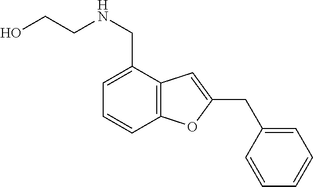 [SMILES: OCCNCc1cccc2oc(Cc3ccccc3)cc12] | ¹H NMR (400 MHz, DMSO-d₆) δ (ppm): 8.25 (s, 1H; HCOOH), 7.39 (d, J = 7.2 Hz, 1H), 7.36-7.31 (m, 4H), 7.27-7.24 (m, 1H), 7.23-7.17 (m, 2H), 6.78 (d, J = 0.8 Hz, 1H), 4.15 (s, 2H), 4.01 (s, 2H), 3.52 (t, J = 5.6 Hz, 2H), 2.69 (t, J = 5.6 Hz, 2H). MS (ESI) m/z 282.2 [C₁₈H₁₉NO₂ + H]⁺. |
| 5 | A | 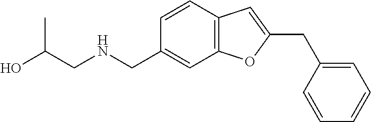 [SMILES: CC(O)CNCc1ccc2cc(Cc3ccccc3)oc2c1] | ¹H NMR (400 MHz, DMSO-d₆) δ (ppm): 8.25 (s, 1H; HCOOH), 7.50-7.48 (m, 2H), 7.36-7.30 (m, 4H), 7.27-7.23 (m, 1H), 7.19 (d, J = 8.0 Hz, 1H), 6.58 (s, 1H), 4.14 (s, 2H), 3.88 (s, 2H), 3.76-3.71 (m, 1H), |
| | | | 2.50-2.49 (m, 1H), 2.47- |
| | | | 2.42 (m, 1H), 1.02 (d, J = |
| | | | 6.4 Hz, 3H). |
| | | | MS (ESI) m/z 296.2 |
| | | | [C₁₉H₂₁NO₂ + H]⁺. |
| 6 | A | 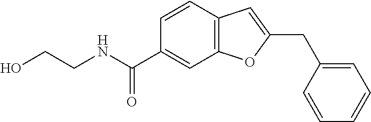 [SMILES: O=C(NCCO)c1ccc2cc(Cc3ccccc3)oc2c1] | ¹H NMR (400 MHz, DMSO-d₆) δ (ppm): 8.40 (t, J = 5.6 Hz, 1H), 7.97 (s, 1H), 7.73 (dd, J = 8.0 Hz, 1.2 Hz, 1H), 7.59 (d, J = 8.4 Hz, 1H), 7.37-7.32 (m, 4H), 7.28-7.25 (m, 1H), 6.66 (s, 1H), 4.71 (t, J = 5.6 Hz, 1H), |
| | | | 4.19 (s, 2H), 3.51 (q, J = 6.0 |
| | | | Hz, 2H), 3.34 (t, J = 5.6 Hz, |
| | | | 2H). |
| | | | MS (ESI) m/z 296.1 |
| | | | [C₁₈H₁₇NO₃ + H]⁺. |
| 7 | A | 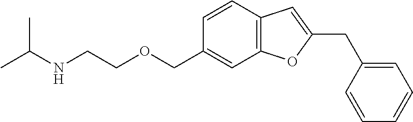 [SMILES: CC(C)NCCOCc1ccc2cc(Cc3ccccc3)oc2c1] | ¹H NMR (400 MHz, DMSO-d₆) δ (ppm): 7.52 (d, J = 8.0 Hz, 1H), 7.47 (s, 1H), 7.34-7.30 (m, 4H), 7.27-7.25 (m, 1H), 7.17 (dd, J = 7.6 Hz, 1.2 Hz, 1H), 6.61 (s, 1H), 4.57 (s, 2H), 4.14 (s, 2H), 3.54 (t, J = 5.6 Hz, 2H), |
| | | | 2.98-2.92 (m, 1H), 2.87 (t, J = |
| | | | 5.6 Hz, 2H), 1.06 (d, J = |
| | | | 6.4 Hz, 6H). |
| | | | MS (ESI) m/z 324.3 |
| | | | [C₂₁H₂₅NO₂ + H]⁺. |
| 8 | A | 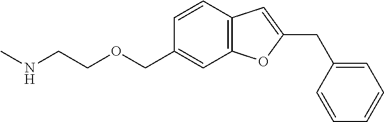 [SMILES: CNCCOCc1ccc2cc(Cc3ccccc3)oc2c1] | ¹H NMR (400 MHz, DMSO-d₆) δ (ppm): 8.32 (s, 1H; HCOOH), 7.51 (d, J = 8.0 Hz, 1H), 7.48 (s, 1H), 7.36-7.30 (m, 4H), 7.27- 7.23 (m, 1H), 7.17 (dd, J = 8.0 Hz, 0.8 Hz, 1H), 6.60 (s, 1H), 4.56 (s, 2H), 4.14 (s, |
| | | | 2H), 3.55 (t, J = 5.2 Hz, 2H), |
| | | | 2.86 (t, J = 5.2 Hz, 2H), 2.40 |
| | | | (s, 3H). |
| | | | MS (ESI) m/z 296.1 |
| | | | [C₁₉H₂₁NO₂ + H]⁺. |
| 9 | B | 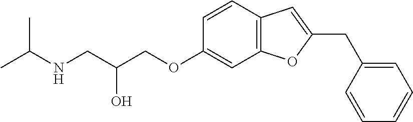 [SMILES: CC(C)NCC(O)COc1ccc2cc(Cc3ccccc3)oc2c1] | ¹H NMR (400 MHz, DMSO-d₆) δ (ppm): 8.34 (br. s, 1H; HCOOH), 7.41 (d, J = 8.4 Hz, 1H), 7.35- 7.29 (m, 4H), 7.26-7.23 (m, 1H), 7.11 (s, 1H), 6.83 (dd, J = 8.4 Hz, 2.4 Hz, 1H), 6.52 (s, 1H), 4.10 (s, 2H), 4.05- |
| | | | 4.01 (m, 1H), 3.96-3.93 (m, |
| | | | 2H), 3.06-3.01 (m, 1H), |
| | | | 2.94-2.90 (m, 1H), 2.80- |
| | | | 2.75 (m, 1H), 1.13-1.11 (m, |
| | | | 6H). |
| | | | MS (ESI) m/z 340.3 |
| | | | [C₂₁H₂₅NO₃ + H]⁺. |
| 10 | A | 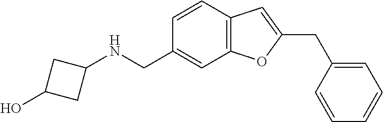 [SMILES: OC1CC(NCc2ccc3cc(Cc4ccccc4)oc3c2)C1] | ¹H NMR (400 MHz, DMSO-d₆) δ (ppm): 8.25 (s, 1H; HCOOH), 7.48-7.45 (m, 2H), 7.35-7.29 (m, 4H), 7.27-7.25 (m, 1H), 7.17 (dd, J = 8.0 Hz, 1.2 Hz, 1H), 6.57 (s, 1H), 4.13 (s, 2H), 3.77 (s, 2H), 3.75-3.69 (m, 1H), |
| | | | 2.74-2.70 (m, 1H), 2.42- |
| | | | 2.35 (m, 2H), 2.07-1.91 (m, |
| | | | 1H), 1.67-1.60 (m, 2H). |
| | | | Mixture of isomers |
| | | | observed in NMR. |
| | | | MS (ESI) m/z 308.2 |
| | | | [C₂₀H₂₁NO₂ + H]⁺. |
| 11 | C | 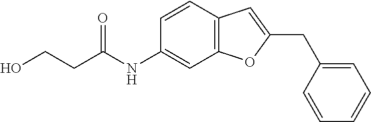 [SMILES: O=C(CCO)Nc1ccc2cc(Cc3ccccc3)oc2c1] | ¹H NMR (400 MHz, DMSO-d₆) δ (ppm): 9.98 (s, 1H), 7.96 (s, 1H), 7.42 (d, J = 8.4 Hz, 1H), 7.35-7.30 (m, 4H), 7.27-7.23 (m, 2H), 6.51 (s, 1H), 4.67 (t, J = 5.2 Hz, 1H), 4.11 (s, 2H), 3.71 (q, J = 5.6 Hz, 2H), 2.46 (t, |
| | | | J = 6.4 Hz, 2H). |
| | | | MS (ESI) m/z 296.1 |
| | | | [C₁₈H₁₇NO₃ + H]⁺. |
| 12 | D | 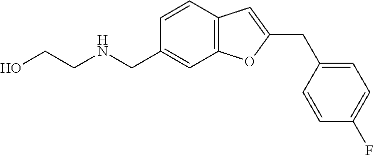 [SMILES: OCCNCc1ccc2cc(Cc3ccc(F)cc3)oc2c1] | ¹H NMR (400 MHz, DMSO-d₆) δ (ppm): 8.24 (s, 1H; HCOOH), 7.49-7.48 (m, 2H), 7.37-7.33 (m, 2H), 7.20-7.14 (m, 3H), 6.57 (s, 1H), 4.13 (s, 2H), 3.87 (s, 2H), 3.49 (t, J = 5.6 Hz, 2H), 2.63 (t, J = 5.6 Hz, 2H). MS (ESI) m/z 300.2 [C₁₈H₁₈FNO₂ + H]⁺. |
| 13 | D | 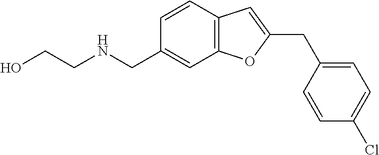 [SMILES: OCCNCc1ccc2cc(Cc3ccc(Cl)cc3)oc2c1] | ¹H NMR (400 MHz, DMSO-d₆) δ (ppm): 8.20 (s, 1H; HCOOH), 7.50-7.48 (m, 2H), 7.39 (d, J = 8.4 Hz, 2H), 7.33 (d, J = 8.4 Hz, 2H), 7.20 (d, J = 8.0 Hz, 1H), 6.60 (s, 1H), 4.15 (s, 2H), 3.89 (s, 2H), 3.49 (t, J = 5.6 Hz, 2H), 2.63 (t, J = 5.6 Hz, 2H). |
| | | | MS (ESI) m/z 316.2 |
| | | | [C₁₈H₁₈ClNO₂ + H]⁺. |
| 15 | D | 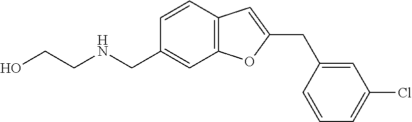 [SMILES: OCCNCc1ccc2cc(Cc3cccc(Cl)c3)oc2c1] | ¹H NMR (400 MHz, DMSO-d₆) δ (ppm): 8.24 (s, 1H; HCOOH), 7.54-7.52 (m, 2H), 7.42-7.41 (m, 1H), 7.38 (d, J = 7.6 Hz, 1H), 7.36-7.34 (m, 1H), 7.32- 7.30 (m, 1H), 7.24 (dd, J = 8.0 Hz, 0.8 Hz, 1H), 6.65 (s, |
| | | | 1H), 4.19 (s, 2H), 3.93 (s, |
| | | | 2H), 3.53 (t, J = 5.6 Hz, 2H), |
| | | | 2.68 (t, J = 5.6 Hz, 2H). |
| | | | MS (ESI) m/z 316.2 |
| | | | [C₁₈H₁₈ClNO₂ + H]⁺. |
| 16 | E | 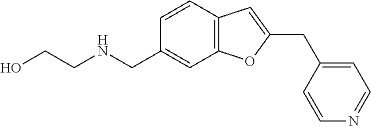 [SMILES: OCCNCc1ccc2cc(Cc3ccncc3)oc2c1] | ¹H NMR (400 MHz, DMSO-d₆) δ (ppm): 8.51 (d, J = 6.0 Hz, 2H), 7.47 (d, J = 8.0 Hz, 1H), 7.44 (s, 1H), 7.32 (d, J = 5.6 Hz, 2H), 7.17 (d, J = 8.0 Hz, 1H), 6.65 (s, 1H), 4.43 (br. s, 1H), 4.19 (s, 2H), 3.77 (s, 2H), |
| | | | 3.47-3.43 (m, 2H), 2.54 (t, J = |
| | | | 5.6 Hz, 2H). |
| | | | MS (ESI) m/z 283.1 |
| | | | [C₁₇H₁₈N₂O₂ + H]⁺. |
| 17 | D | 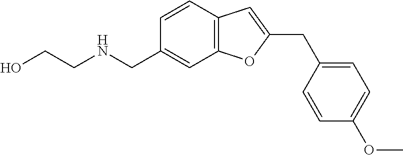 [SMILES: COc1ccc(Cc2cc3ccc(CNCCO)cc3o2)cc1] | ¹H NMR (400 MHz, DMSO-d₆) δ (ppm): 8.20 (s, 1H; HCOOH), 7.50-7.48 (m, 2H), 7.24-7.19 (m, 3H), 6.89 (d, J = 8.4 Hz, 2H), 6.54 (s, 1H), 4.06 (s, 2H), 3.91 (s, 2H), 3.73 (s, 3H), 3.50 (t, J = 5.6 Hz, 2H), 2.66 (t, J = 5.6 Hz, 2H). MS (ESI) m/z 312.2 |
| | | | [C₁₉H₂₁NO₃ + H]⁺. |
| 18 | F | 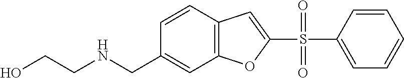 [SMILES: O=S(=O)(c1ccccc1)c1cc2ccc(CNCCO)cc2o1] | ¹H NMR (400 MHz, DMSO-d₆) δ (ppm): 8.16 (s, 1H; HCOOH), 8.05-8.03 (m, 2H), 7.93 (s, 1H), 7.81- 7.78 (m, 2H), 7.75 (s, 1H), |
| | | | 7.72-7.69 (m, 2H), 7.46- |
| | | | 7.44 (m, 1H), 4.01 (s, 2H), |
| | | | 3.52 (t, J = 5.6 Hz, 2H), 2.69 |
| | | | (t, J = 5.6 Hz, 2H). |
| | | | MS (ESI) m/z 332.2 |
| | | | [C₁₇H₁₇NO₄S + H]⁺. |
| 19 | G | 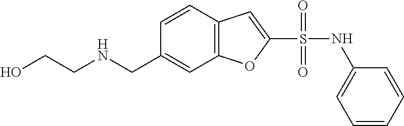 [SMILES: O=S(=O)(Nc1ccccc1)c1cc2ccc(CNCCO)cc2o1] | ¹H NMR (400 MHz, DMSO-d₆) δ (ppm): 8.20 (s, 1H; HCOOH), 7.70-7.68 (m, 2H), 7.49 (s, 1H), 7.36 (d, J = 9.2 Hz, 1H), 7.22- 7.18 (m, 2H), 7.12-7.10 (m, 2H), 6.96 (t, J = 7.2 Hz, 1H), 3.98 (s, 2H), 3.52 (t, J = 5.6 |
| | | | Hz, 2H), 2.69 (t, J = 5.6 Hz, |
| | | | 2H). |
| | | | MS (ESI) m/z: 347.1 |
| | | | [C₁₇H₁₈N₂O₄S + H]⁺. |
| 23 | D | 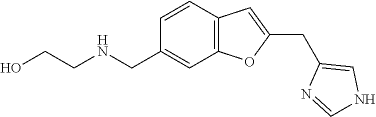 [SMILES: OCCNCc1ccc2cc(Cc3c[nH]cn3)oc2c1] | ¹H NMR (400 MHz, DMSO-d₆) δ (ppm): 11.88 (br. s, 1H), 7.56 (d, J = 1.2 Hz, 1H), 7.47-7.45 (m, 2H), 7.17 (d, J = 8.0 Hz, 1H), 6.91 (br. s, 1H), 6.51 (s, 1H), 4.60 (br. s, 1H), 4.02 (s, 2H), 3.86 (s, 2H), 3.49 (t, J = 5.6 |
| | | | Hz, 2H), 2.62 (t, J = 5.6 Hz, |
| | | | 2H). |
| | | | MS (ESI) m/z 270.0 |
| | | | [C₁₅H₁₇N₃O₂ − H]⁻. |
| 25 | D | 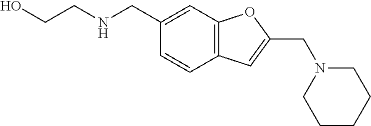 [SMILES: OCCNCc1ccc2cc(CN3CCCCC3)oc2c1] | ¹H NMR (400 MHz, DMSO-d₆) δ (ppm): 8.24 (s, 2H; 2HCOOH), 7.56 (s, 1H), 7.53 (d, J = 7.6 Hz, 1H), 7.24 (dd, J = 8.0 Hz, 1.2 Hz, 1H), 6.72 (s, 1H), 3.95 (s, 2H), 3.60 (s, 2H), 3.53 (t, J = 5.6 Hz, 2H), 2.70 |
| | | | (t, J = 5.6 Hz, 2H), 2.40 (m, |
| | | | 4H), 1.51-1.48 (m, 4H), |
| | | | 1.39-1.35 (m, 2H). |
| | | | MS (ESI) m/z 289.1 |
| | | | [C₁₇H₂₄N₂O₂ + H]⁺. |
| 28 | D | 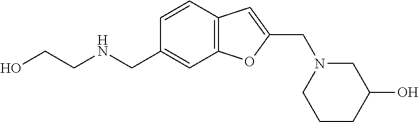 [SMILES: OCCNCc1ccc2cc(CN3CCCC(O)C3)oc2c1] | ¹H NMR (400 MHz, DMSO-d₆/D₂O exchange) δ (ppm): 8.20 (s, 1H; HCOH), 7.72 (s, 1H), 7.69 (d, J = 8.0 Hz, 1H), 7.37 (d, J = 8.0 Hz, 1H), 6.90 (s, 1H), 4.26 (s, 2H), 3.83-3.78 (m, 2H), 3.68 (t, J = 5.2 Hz, |
| | | | 2H), 3.59-3.54 (m, 1H), |
| | | | 3.00 (t, J = 5.2 Hz, 2H), |
| | | | 2.93-2.91 (m, 1H), 2.79- |
| | | | 2.76 (m, 1H), 2.16 (t, J = 5.6 |
| | | | Hz, 1H), 2.01 (t, J = 5.6 Hz, |
| | | | 1H), 1.81-1.77 (m, 1H), |
| | | | 1.71-1.67 (m, 1H), 1.51- |
| | | | 1.45 (m, 1H), 1.18-1.10 (m, |
| | | | 1H). |
| | | | MS (ESI) m/z 305.2 |
| | | | [C₁₇H₂₄N₂O₃ + H]⁺. |
| 29 | H | 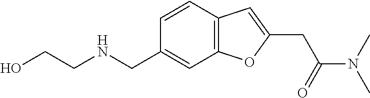 [SMILES: CN(C)C(=O)Cc1cc2ccc(CNCCO)cc2o1] | ¹H NMR (400 MHz, DMSO-d₆) δ (ppm): 8.27 (s, 1H; HCOOH), 7.52-7.50 (m, 2H), 7.22 (d, J = 8.4 Hz, 1H), 6.67 (s, 1H), 3.94 (s, 2H), 3.92 (s, 2H), 3.51 (t, J = |
| | | | 5.6 Hz, 2H), 3.07 (s, 3H), |
| | | | 2.85 (s, 3H), 2.66 (t, J = 5.6 |
| | | | Hz, 2H). |
| | | | MS (ESI) m/z 277.1 |
| | | | [C₁₅H₂₀N₂O₃ + H]⁺. |
| 30 | H | 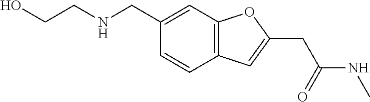 [SMILES: CNC(=O)Cc1cc2ccc(CNCCO)cc2o1] | ¹H NMR (400 MHz, DMSO-d₆) δ (ppm): 8.03 (s, 1H), 7.50-7.44 (m, 2H), 7.16 (d, J = 8.0 Hz, 1H), 6.63 (s, 1H), 4.44 (br. s, 1H), 3.78 (s, 2H), 3.64 (s, 2H), 3.45 (t, J = 5.6 Hz, 2H), 2.61 |
| | | | (d, J = 4.4 Hz, 3H), 2.55 (t, |
| | | | J = 5.6 Hz, 2H). |
| | | | MS (ESI) m/z 263.2 |
| | | | [C₁₄H₁₈N₂O₃ + H]⁺. |
| 31 | D | 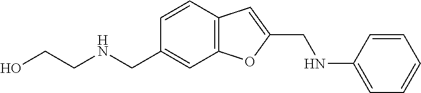 [SMILES: OCCNCc1ccc2cc(CNc3ccccc3)oc2c1] | ¹H NMR (400 MHz, DMSO-d₆) δ (ppm): 8.27 (s, 1H; HCOOH), 7.55 (s, 1H), 7.51 (d, J = 8.0 Hz, 1H), 7.22 (dd, J = 8.0 Hz, 1.2 Hz, 1H), 7.08-7.04 (m, 2H), |
| | | | 6.71 (d, J = 0.8, 1H), 6.67 |
| | | | (dd, J = 8.8 Hz, 1.2 Hz, 2H), |
| | | | 6.54 (t, J = 7.2 Hz, 1H), 6.25 |
| | | | (br. s, 1H), 4.41 (d, J = 4.4 |
| | | | Hz, 2H), 3.94 (s, 2H), 3.52 |
| | | | (t, J = 5.6 Hz, 2H), 2.68 (t, J = |
| | | | 5.6 Hz, 2H). |
| | | | MS (ESI) m/z 297.1 |
| | | | [C₁₈H₂₀N₂O₂ + H]⁺. |
| 32 | D | 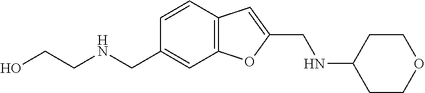 [SMILES: OCCNCc1ccc2cc(CNC3CCOCC3)oc2c1] | ¹H NMR (400 MHz, DMSO-d₆) δ (ppm): 8.25 (s, 2H: HCOOH), 7.58 (s, 1H), 7.54 (d, J = 8.0 Hz, 1H), 7.25 (d, J = 8.0 Hz, 1H), 6.72 (s, 1H), 4.00 (s, 2H), |
| | | | 3.89 (s, 2H), 3.83-3.80 (m, |
| | | | 2H), 3.55 (t, J = 5.6 Hz, |
| | | | 2H), 3.28-3.22 (m, 2H), |
| | | | 2.74 (t, J = 5.6 Hz, 2H), |
| | | | 2.68-2.63 (m, 1H), 1.80- |
| | | | 1.77 (m, 2H), 1.32-1.23 (m, |
| | | | 2H). |
| | | | MS (ESI) m/z 305.1 |
| | | | [C₁₇H₂₄N₂O₃ + H]⁺. |
| 33 | D | 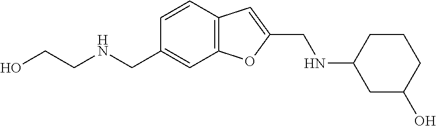 [SMILES: OCCNCc1ccc2cc(CNC3CCCC(O)C3)oc2c1] | ¹H NMR (400 MHz, DMSO-d₆) δ (ppm): 8.32 (br. s, 2H; HCOOH), 7.50- 7.48 (m, 2H), 7.19 (d, J = 8.0 Hz, 1H), 6.67 (s, 1H), 3.87-3.84 (m, 4H), 3.50 (t, J = 5.6 Hz, 2H), 3.36-3.31 (m, 1H), 2.86-2.84 (m, 1H), |
| | | | 2.67-2.63 (m, 2H), 2.44- |
| | | | 2.40 (m, 1H), 1.82-1.73 (m, |
| | | | 1H), 1.65-1.62 (m, 1H), |
| | | | 1.49-1.37 (m, 2H), 1.18- |
| | | | 0.87 (m, 3H). Mixture of |
| | | | isomers observed in NMR. |
| | | | MS (ESI) m/z 319.2 |
| | | | [C₁₈H₂₆N₂O₃ + H]⁺. |
| 34 | D | 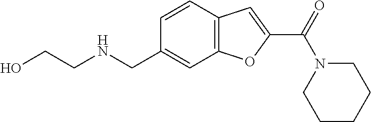 [SMILES: O=C(c1cc2ccc(CNCCO)cc2o1)N1CCCCC1] | ¹H NMR (400 MHz, DMSO-d₆) δ (ppm): 8.23 (s, 1H; HCOOH), 7.67 (d, J = 8.4 Hz, 1H), 7.64 (s, 1H), 7.34-7.31 (m, 2H), 3.92 (s, 2H), 3.64 (br. s, 4H), 3.50 (t, J = 5.6 Hz, 2H), 2.64 (t, J = 5.6 Hz, 2H), 1.66-1.64 (m, |
| | | | 2H), 1.60-1.54 (m, 4H). |
| | | | MS (ESI) m/z 303.1 |
| | | | [C₁₇H₂₂N₂O₃ + H]⁺. |
| 35 | D | 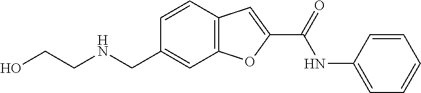 [SMILES: O=C(Nc1ccccc1)c1cc2ccc(CNCCO)cc2o1] | ¹H NMR (400 MHz, DMSO-d₆) δ (ppm): 10.51 (s, 1H), 8.25 (s, 1H; HCOOH), 7.81 (d, J = 7.6 Hz, 2H), 7.78 (d, J = 8.4 Hz, 1H), 7.75 (d, J = 7.6 Hz, |
| | | | 2H), 7.40-7.36 (m, 3H), |
| | | | 7.13 (t, J = 7.6 Hz, 1H), 4.00 |
| | | | (s, 2H), 3.54 (t, J = 5.6 Hz, |
| | | | 2H), 2.70 (t, J = 5.6 Hz, 2H). |
| | | | MS (ESI) m/z 311.2 |
| | | | [C₁₈H₁₈N₂O₃ + H]⁺. |
| 36 | A | 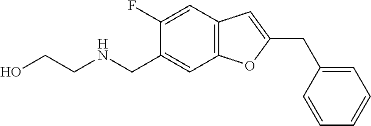 [SMILES: OCCNCc1cc2oc(Cc3ccccc3)cc2cc1F] | ¹H NMR (400 MHz, DMSO-d₆) δ (ppm): 8.22 (s, 1H, HCOOH), 7.56 (d, J = 6.0 Hz, 1H), 7.36-7.30 (m, 5H), 7.27-7.25 (m, 1H), 6.58 (s, 1H), 4.14 (s, 2H), 3.84 (s, 2H), 3.48 (t, J = 5.6 Hz, 2H), 2.62 (t, J = 5.6 Hz, |
| | | | 2H). |
| | | | MS (ESI) m/z 300.1 |
| | | | [C₁₈H₁₈FNO₂ + H]⁺. |
| 37 | A | 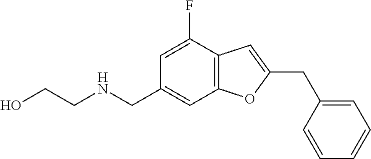 [SMILES: OCCNCc1cc(F)c2cc(Cc3ccccc3)oc2c1] | ¹H NMR (400 MHz, DMSO-d₆) δ (ppm): 8.24 (s, 1H; HCOOH), 7.39 (s, 1H), 7.36-7.30 (m, 4H), 7.28- 7.24 (m, 1H), 7.09 (d, J = 10.4 Hz, 1H), 6.72 (s, 1H), 4.16 (s, 2H), 3.89 (s, 2H), 3.50 (t, J = 5.6 Hz, 2H), 2.63 (t, J = 5.6 Hz, 2H). MS (ESI) m/z 300.2 |
| | | | [C₁₈H₁₈FNO₂ + H]⁺. |
| 38 | I | 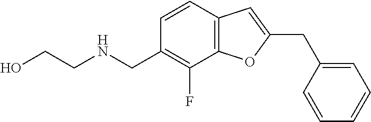 [SMILES: OCCNCc1ccc2cc(Cc3ccccc3)oc2c1F] | ¹H NMR (400 MHz, DMSO-d₆) δ (ppm): 8.26 (s, 1H; HCOOH), 7.37-7.30 (m, 5H), 7.28-7.26 (m, 2H), 6.70 (d, J = 3.2 Hz, 1H), 4.18 (s, 2H), 3.96 (s, 2H), 3.51 (t, J = 5.6 Hz, 2H), 2.68 (t, J = 5.6 Hz, 2H). |
| | | | MS (ESI) m/z 300.2 |
| | | | [C₁₈H₁₈FNO₂ + H]⁺. |
| 42 | I | 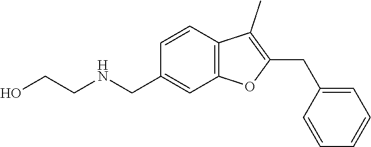 [SMILES: Cc1c(Cc2ccccc2)oc2cc(CNCCO)ccc12] | ¹H NMR (400 MHz, DMSO-d₆) δ (ppm): 8.29 (s, 1H; HCOOH), 7.49-7.47 (m, 2H), 7.32-7.20 (m, 6H), 4.12 (s, 2H), 3.95 (s, 2H), 3.52 (t, J = 5.6 Hz, 2H), 2.68 (t, J = 5.6 Hz, 2H), 2.23 (s, 3H). MS (ESI) m/z 296.2 |
| | | | [C₁₉H₂₁NO₂ + H]⁺. |
| 43 | J | 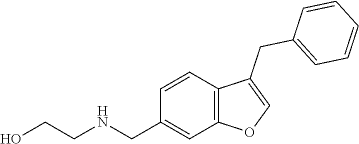 [SMILES: OCCNCc1ccc2c(Cc3ccccc3)coc2c1] | ¹H NMR (400 MHz, DMSO-d₆) δ (ppm): 8.27 (s, 1H; HCOOH), 7.83 (s, 1H), 7.57 (s, 1H), 7.43 (d, J = 8.0 Hz, 1H), 7.33-7.27 (m, 4H), 7.21-7.17 (m, 2H), 4.01 (s, 2H), 3.93 (s, 2H), 3.51 (t, J = 5.6 Hz, 2H), 2.66 (t, J = 5.6 Hz, 2H). |
| | | | MS (ESI) m/z 282.2 |
| | | | [C₁₈H₁₉NO₂ + H]⁺. |
| 45 | A | 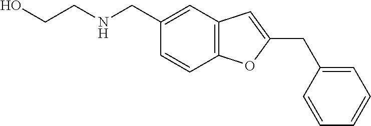 [SMILES: OCCNCc1ccc2oc(Cc3ccccc3)cc2c1] | ¹H NMR (400 MHz, DMSO-d₆) δ (ppm): 8.30 (s, 1H; HCOOH), 7.54 (s, 1H), 7.43 (d, J = 8.4 Hz, 1H), 7.36-7.31 (m, 4H), 7.28- 7.22 (m, 2H), 6.61 (s, 1H), 4.14 (s, 2H), 3.87 (s, 2H), 3.51 (t, J = 5.6 Hz, 2H), 2.65 |
| | | | (t, J = 5.6 Hz, 2H). |
| | | | MS (ESI) m/z 282.1 |
| | | | [C₁₈H₁₉NO₂ + H]⁺. |
| 47 | K | 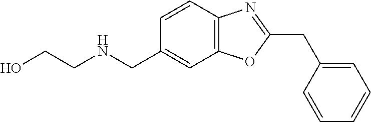 [SMILES: OCCNCc1ccc2nc(Cc3ccccc3)oc2c1] | ¹H NMR (400 MHz, DMSO-d₆) δ (ppm): 8.28 (s, 1H; HCOOH), 7.65 (s, 1H), 7.62 (d, J = 8.4 Hz, 1H), 7.37-7.33 (m, 5H), 7.30- 7.27 (m, 1H), 4.32 (s, 2H), 3.89 (s, 2H), 3.49 (t, J = 5.6 Hz, 2H), 2.61 (t, J = 5.6 Hz, |
| | | | 2H). |
| | | | MS (ESI) m/z 283.2 |
| | | | [C₁₇H₁₈N₂O₂ + H]⁺. |
| 48 | K | 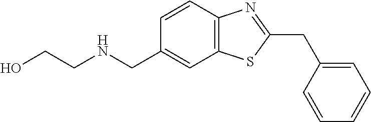 [SMILES: OCCNCc1ccc2nc(Cc3ccccc3)sc2c1] | ¹H NMR (400 MHz, DMSO-d₆) δ (ppm): 7.98 (s, 1H), 7.90 (d, J = 8.4 Hz, 1H), 7.48 (d, J = 8.8 Hz, 1H), 7.40-7.34 (m, 4H), 7.30-7.28 (m, 1H), 4.46 (s, 2H), 3.92 (s, 2H), 3.50 (t, J = 5.6 Hz, 2H), 2.65 (t, J = |
| | | | 5.6 Hz, 2H). |
| | | | MS (ESI) m/z 299.1 |
| | | | [C₁₇H₁₈N₂OS + H]⁺. |
| 49 | D | 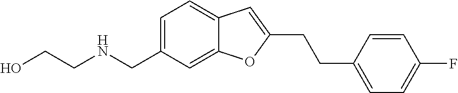 [SMILES: OCCNCc1ccc2cc(CCc3ccc(F)cc3)oc2c1] | ¹H NMR (400 MHz, DMSO-d₆) δ (ppm): 8.24 (s, 1H; HCOOH), 7.51 (s, 1H), 7.46 (d, J = 8.0 Hz, 1H), 73.1-7.27 (m, 2H), 7.19 (dd, J = 8.0 Hz, 0.8 Hz, 1H), |
| | | | 7.11-7.07 (m, 2H), 6.54 (s, |
| | | | 1H), 3.91 (s, 2H), 3.52 (t, J = |
| | | | 5.6 Hz, 2H), 3.09-3.06 |
| | | | (m, 2H), 3.00-2.98 (m, 2H), |
| | | | 2.67 (t, J = 5.6 Hz, 2H). |
| | | | MS (ESI) m/z 314.3 |
| | | | [C₁₉H₂₀FNO₂ + H]⁺. |
| 50 | L | 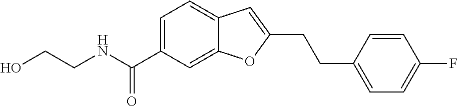 [SMILES: O=C(NCCO)c1ccc2cc(CCc3ccc(F)cc3)oc2c1] | ¹H NMR (400 MHz, DMSO-d₆) δ (ppm): 8.42 (t, J = 5.6 Hz, 1H), 7.99 (s, 1H), 7.73 (dd, J = 8.0 Hz, 1.2 Hz, 1H), 7.57 (d, J = 8.0 Hz, 1H), 7.31-7.27 (m, 2H), 7.11-7.07 (m, 2H), 6.64 (s, |
| | | | 1H), 4.73 (t, J = 5.6 Hz, 1H), |
| | | | 3.52 (q, J = 6.0 Hz, 2H), |
| | | | 3.37-3.34 (m, 2H), 3.14- |
| | | | 3.10 (m, 2H), 3.05-3.01 (m, |
| | | | 2H). |
| | | | MS (ESI) m/z 328.2 |
| | | | [C₁₉H₁₈FNO₃ + H]⁺. |
| 72 | A | 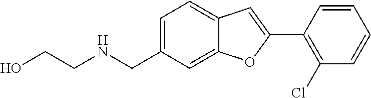 [SMILES: OCCNCc1ccc2cc(-c3ccccc3Cl)oc2c1] | ¹H NMR (400 MHz, DMSO-d₆) δ (ppm): 8.25 (s, 1H; HCOOH), 8.02 (dd, J = 8.0 Hz, 2.0 Hz, 1H), 7.70- 7.63 (m, 3H), 7.58 (s, 1H), 7.55-7.51 (m, 1H), 7.48- 7.43 (m, 1H), 7.33-7.31 (m, |
| | | | 1H), 3.97 (s, 2H), 3.53 (t, J = |
| | | | 6.0 Hz, 2H), 2.69 (t, J = |
| | | | 6.0 Hz, 2H). |
| | | | MS (ESI) m/z 302.0 |
| | | | [C₁₇H₁₆ClNO₂ + H]⁺. |
| 73 | A | 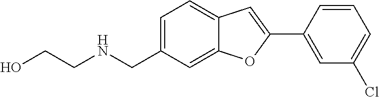 [SMILES: OCCNCc1ccc2cc(-c3cccc(Cl)c3)oc2c1] | ¹H NMR (400 MHz, DMSO-d₆) δ (ppm): 8.25 (s, 1H; HCOOH), 7.97 (t, J = 2.0 Hz, 1H), 7.88 (dt, J = 8.8 Hz, 1.2 Hz, 1H), 7.67 (s, 1H), 7.63 (d, J = 8.0 Hz, 1H), 7.56 (d, J = 0.8 Hz, |
| | | | 1H), 7.53 (d, J = 8.0 Hz, |
| | | | 1H), 7.48-7.45 (m, 1H), |
| | | | 7.30 (dd, J = 8.0 Hz, 1.2 Hz, |
| | | | 1H), 3.97 (s, 2H), 3.53 (t, J = |
| | | | 5.6 Hz, 2H), 2.69 (t, J = |
| | | | 5.6 Hz, 2H). |
| | | | MS (ESI) m/z 302.0 |
| | | | [C₁₇H₁₆ClNO₂ + H]⁺. |
| 74 | A | 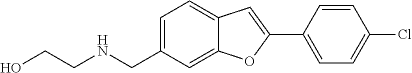 [SMILES: OCCNCc1ccc2cc(-c3ccc(Cl)cc3)oc2c1] | ¹H NMR (400 MHz, DMSO-d₆) δ (ppm): 8.22 (s, 1H; HCOOH), 7.95-7.93 (m, 2H), 7.67 (s, 1H), 7.63 (d, J = 8.0 Hz, 1H), 7.59- |
| | | | 7.57 (m, 2H), 7.49 (s, 1H), |
| | | | 7.30 (d, J = 8.0 Hz, 1H), |
| | | | 3.98 (s, 2H), 3.54 (t, J = 5.6 |
| | | | Hz, 2H), 2.71 (t, J = 5.6 Hz, |
| | | | 2H). |
| | | | MS (ESI) m/z 302.1 |
| | | | [C₁₇H₁₆ClNO₂ + H]⁺. |
| 75 | A | 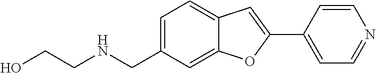 [SMILES: OCCNCc1ccc2cc(-c3ccncc3)oc2c1] | ¹H NMR (400 MHz, DMSO-d₆) δ (ppm): 8.68 (dd, J = 4.8 Hz, 1.6 Hz, 2H), 7.85 (dd, J = 4.4 Hz, 1.6 Hz, 2H), 7.73 (s, 1H), 7.66-7.64 |
| | | | (m, 2H), 7.28 (dd, J = 8.0 |
| | | | Hz, 1.6 Hz, 1H), 4.46 (t, J = |
| | | | 5.2 Hz, 1H), 3.85 (s, 2H), |
| | | | 3.48 (q, J = 5.6 Hz, 2H), |
| | | | 2.58 (t, J = 5.6 Hz, 2H), 2.22 |
| | | | (br. s, 1H). |
| | | | MS (ESI) m/z 269.0 |
| | | | [C₁₆H₁₆N₂O₂ + H]⁺. |
| 76 | A | 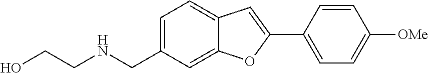 [SMILES: COc1ccc(-c2cc3ccc(CNCCO)cc3o2)cc1] | ¹H NMR (400 MHz, DMSO-d₆) δ (ppm): 8.27 (br. s, 1H; HCOOH), 7.85 (d, J = 8.4 Hz, 2H), 7.63 (s, 1H), 7.57 (d, J = 8.0 Hz, |
| | | | 1H), 7.28-7.25 (m, 2H), |
| | | | 7.07 (d, J = 8.4 Hz, 2H), |
| | | | 3.96 (s, 2H), 3.83 (s, 3H), |
| | | | 3.54 (t, J = 5.6 Hz, 2H), 2.71 |
| | | | (t, J = 5.2 Hz, 2H). |
| | | | MS (ESI) m/z 298.1 |
| | | | [C₁₈H₁₉NO₃ + H]⁺. |
| 77 | A | 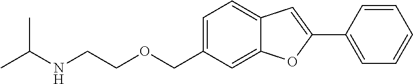 [SMILES: CC(C)NCCOCc1ccc2cc(-c3ccccc3)oc2c1] | ¹H NMR (400 MHz, DMSO-d₆) δ (ppm): 8.33 (s, 1H; HCOOH), 7.93-7.91 (m, 2H), 7.65-7.63 (m, 2H), 7.53-7.49 (m, 2H), 7.44- 7.39 (m, 2H), 7.27-7.24 (m, |
| | | | 1H), 4.63 (s, 2H), 3.60 (t, J = |
| | | | 5.6 Hz, 2H), 3.00-2.94 |
| | | | (m, 1H), 2.89 (t, J = 5.6 Hz, |
| | | | 2H), 1.08 (d, J = 6.4 Hz, |
| | | | 6H). |
| | | | MS (ESI) m/z 310.2 |
| | | | [C₂₀H₂₃NO₂ + H]⁺. |
| 78 | B | 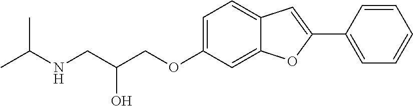 [SMILES: CC(C)NCC(O)COc1ccc2cc(-c3ccccc3)oc2c1] | ¹H NMR (400 MHz, DMSO-d₆) δ (ppm): 8.38 (br. s, 1H; HCOOH), 7.87- 7.85 (m, 2H), 7.55-7.47 (m, 3H), 7.39-7.35 (m, 2H), 7.28 (d, J = 2.0 Hz, 1H), 6.92 (dd, J = 8.4 Hz, 2.0 Hz, |
| | | | 1H), 4.14-4.09 (m, 1H), |
| | | | 4.04-4.02 (m, 2H), 3.14- |
| | | | 3.10 (m, 1H), 3.00 (dd, J = |
| | | | 12.4 Hz, 3.6 Hz, 1H), 2.88- |
| | | | 2.82 (m, 1H), 1.18-1.16 (m, |
| | | | 6H). |
| | | | MS (ESI) m/z 326.2 |
| | | | [C₂₀H₂₃NO₃ + H]⁺. |
| 79 | A | 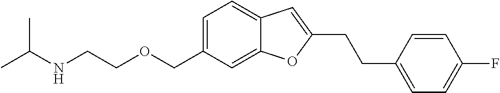 [SMILES: CC(C)NCCOCc1ccc2cc(CCc3ccc(F)cc3)oc2c1] | ¹H NMR (400 MHz, DMSO-d₆) δ (ppm): 7.49- 7.46 (m, 2H), 7.31-7.27 (m, 2H), 7.17-7.14 (m, 1H), 7.11-7.07 (m, 2H), 6.55 (s, 1H), 4.55 (s, 2H), 3.49 (t, J = |
| | | | 6.0 Hz, 2H), 3.08-3.01 |
| | | | (m, 4H), 2.75-2.71 (m, 3H), |
| | | | 0.98 (d, J = 6.4 Hz, 6H). |
| | | | MS (ESI) m/z 356.2 |
| | | | [C₂₂H₂₆FNO₂ + H]⁺. |
| 80 | B | 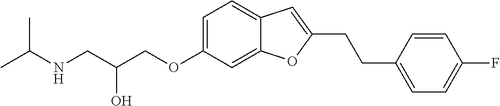 [SMILES: CC(C)NCC(O)COc1ccc2cc(CCc3ccc(F)cc3)oc2c1] | ¹H NMR (400 MHz, DMSO-d₆) δ (ppm): 7.36 (d, J = 8.4 Hz, 1H), 7.30-7.26 (m, 2H), 7.11-7.06 (m, 3H), 6.81 (dd, J = 8.4 Hz, 2.0 Hz, 1H), 6.45 (d, J = 0.8 Hz, 1H), 4.99 (br. s, 1H), 3.99- |
| | | | 3.95 (m, 1H), 3.90-3.85 (m, |
| | | | 2H), 3.06-2.97 (m, 4H), |
| | | | 2.73-2.67 (m, 2H), 2.58- |
| | | | 2.52 (m, 1H), 0.99-0.97 (m, |
| | | | 6H). |
| | | | MS (ESI) m/z 372.6 |
| | | | [C₂₂H₂₆FNO₃ + H]⁺. |
| 105 | A | 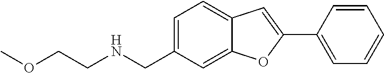 [SMILES: COCCNCc1ccc2cc(-c3ccccc3)oc2c1] | ¹H NMR (400 MHz, DMSO-d₆) δ (ppm): 7.92- 7.90 (m, 2H), 7.58-7.56 (m, 2H), 7.51 (t, J = 7.2 Hz, 2H), 7.42-7.38 (m, 2H), 7.23 (d, J = |
| | | | 8.0 Hz, 1H), 3.82 (s, 2H), |
| | | | 3.41 (t, J = 5.6 Hz, 2H), 3.24 |
| | | | (s, 3H), 2.65 (t, J = 6.0 Hz, |
| | | | 2H), 2.16 (br. s, 1H). |
| | | | MS (ESI) m/z 282.1 |
| | | | [C₁₈H₁₉NO₂ + H]⁺. |
| 106 | K | 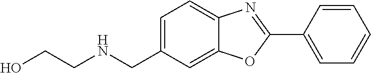 [SMILES: OCCNCc1ccc2nc(-c3ccccc3)oc2c1] | ¹H NMR (400 MHz, DMSO-d₆) δ (ppm): 8.27 (s, 1H; HCOOH), 8.23-8.20 (m, 2H), 7.83 (s, 1H), 7.78 (d, J = 8.4 Hz, 1H), 7.68- |
| | | | 7.60 (m, 3H), 7.45 (dd, J = |
| | | | 8.4 Hz, 2.0 Hz, 1H), 4.01 (s, |
| | | | 2H), 3.55 (t, J = 5.6 Hz, 2H), |
| | | | 2.71 (t, J = 5.6 Hz, 2H). |
| | | | MS (ESI) m/z 269.0 |
| | | | [C₁₆H₁₆N₂O₂ + H]⁺. |
| 107 | A | 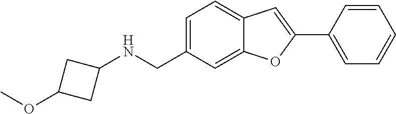 [SMILES: COC1CC(NCc2ccc3cc(-c4ccccc4)oc3c2)C1] | ¹H NMR (400 MHz, DMSO-d₆) δ (ppm): 8.20 (s, 1H; HCOOH), 7.91 (d, J = 8.4 Hz, 2H), 7.60-7.58 (m, 2H), 7.51 (t, J = 7.6 Hz, 2H), 7.42-7.39 (m, 2H), 7.25 (d, J = 8.0 Hz, 1H), 3.81-3.79 (m, 2H), 3.55-3.47 (m, 1H), |
| | | | 3.10 (s, 3H), 2.86-2.82 (m, |
| | | | 1H), 2.47-2.41 (m, 2H), |
| | | | 2.04 (t, J = 5.6 Hz, 1H), |
| | | | 1.68-1.63 (m, 2H). Mixture |
| | | | of isomers observed in |
| | | | NMR |
| | | | MS (ESI) m/z 308.2 |
| | | | [C₂₀H₂₁NO₂ + H]⁺. |
| 108 | A | 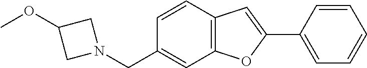 [SMILES: COC1CN(Cc2ccc3cc(-c4ccccc4)oc3c2)C1] | ¹H NMR (400 MHz, DMSO-d₆) δ (ppm): 7.92- 7.90 (m, 2H), 7.57 (d, J = 8.0 Hz, 1H), 7.53-7.49 (m, 3H), 7.42-7.39 (m, 2H), |
| | | | 7.17 (dd, J = 1.2, 8.0 Hz, |
| | | | 1H), 3.99-3.96 (m, 1H), |
| | | | 3.68 (s, 2H), 3.50-3.47 (m, |
| | | | 2H), 3.15 (s, 3H), 2.88-2.85 |
| | | | (m, 2H). |
| | | | MS (ESI) m/z 294.0 |
| | | | [C₁₉H₁₉NO₂ + H]⁺. |
| 109 | A | 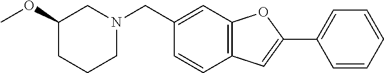 [SMILES: CO[C@@H]1CCCN(Cc2ccc3cc(-c4ccccc4)oc3c2)C1] | ¹H NMR (400 MHz, DMSO-d₆) δ (ppm): 7.93- 7.90 (m, 2H), 7.59 (d, J = 8.0 Hz, 1H), 7.53-7.49 (m, 3H), 7.42-7.39 (m, 2H), |
| | | | 7.21 (dd, J = 8.0 Hz, 0.8 Hz, |
| | | | 1H), 3.61 (s, 2H), 3.27-3.21 |
| | | | (m, 4H), 2.92-2.90 (m, 1H), |
| | | | 2.64-2.61 (m, 1H), 1.97- |
| | | | 1.85 (m, 3H), 1.66-1.62 (m, |
| | | | 1H), 1.47-1.37 (m, 1H), |
| | | | 1.14-1.07 (m, 1H). |
| | | | MS (ESI) m/z 322.2 |
| | | | [C₂₁H₂₃NO₂ + H]⁺. |
| 110 | A | 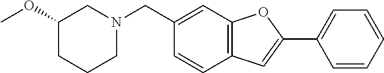 [SMILES: CO[C@H]1CCCN(Cc2ccc3cc(-c4ccccc4)oc3c2)C1] | ¹H NMR (400 MHz, DMSO-d₆) δ (ppm): 7.93- 7.91 (m, 2H), 7.59 (d, J = 8.0 Hz, 1H), 7.53-7.49 (m, 3H), 7.42-7.39 (m, 2H), |
| | | | 7.21 (dd, J = 8.0 Hz, 0.8 Hz, |
| | | | 1H), 3.60 (s, 2H), 3.26-3.21 |
| | | | (m, 4H), 2.92-2.90 (m, 1H), |
| | | | 2.64-2.61 (m, 1H), 1.99- |
| | | | 1.85 (m, 3H), 1.67-1.62 (m, |
| | | | 1H), 1.46-1.37 (m, 1H), |
| | | | 1.13-1.07 (m, 1H). |
| | | | MS (ESI) m/z 322.1 |
| | | | [C₂₁H₂₃NO₂ + H]⁺. |
| 111 | A | 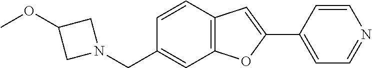 [SMILES: COC1CN(Cc2ccc3cc(-c4ccncc4)oc3c2)C1] | ¹H NMR (400 MHz, DMSO-d₆) δ (ppm): 8.68 (dd, J = 4.8 Hz, 1.2 Hz, 2H), 7.85 (dd, J = 4.4 Hz, 1.2 Hz, 2H), 7.74 (s, 1H), 7.65 (d, J = |
| | | | 8.0 Hz, 1H), 7.56 (s, 1H), |
| | | | 7.23 (d, J = 8.0 Hz, 1H), |
| | | | 3.98 (pent, J = 6.0 Hz, 1H), |
| | | | 3.70 (s, 2H), 3.51-3.47 (m, |
| | | | 2H), 3.15 (s, 3H), 2.89-2.85 |
| | | | (m, 2H). |
| | | | MS (ESI) m/z 295.2 |
| | | | [C₁₈H₁₈N₂O₂ + H]⁺. |
| 112 | A | 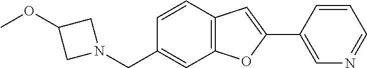 [SMILES: COC1CN(Cc2ccc3cc(-c4cccnc4)oc3c2)C1] | ¹H NMR (400 MHz, DMSO-d₆) δ (ppm): 9.13 (d, J = 1.6 Hz, 1H), 8.59 (dd, J = 4.8 Hz, 1.6 Hz, 1H), 8.26 (dt, J = 8.0 Hz, 1.6 Hz, 1H), |
| | | | 7.61 (d, J = 8.0 Hz, 1H), |
| | | | 7.57-7.52 (m, 3H), 7.20 (d, |
| | | | J = 8.0 Hz, 1H), 3.99-3.97 |
| | | | (m, 1H), 3.69 (s, 2H), 3.49 |
| | | | (dt, J = 6.0 Hz, 2.0 Hz, 2H), |
| | | | 3.15 (s, 3H), 2.87 (dt, J = 6.0 |
| | | | Hz, 2.0 Hz, 2H). |
| | | | MS (ESI) m/z 295.1 |
| | | | [C₁₈H₁₈N₂O₂ + H]⁺. |
| 113 | K | 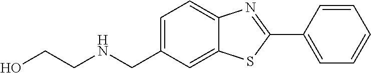 [SMILES: OCCNCc1ccc2nc(-c3ccccc3)sc2c1] | ¹H NMR (400 MHz, DMSO-d₆) δ (ppm): 8.22 (s, 1H; HCOOH), 8.12-8.08 (m, 3H), 8.02 (d, J = 8.4 Hz, 1H), 7.60-7.54 (m, 4H), |
| | | | 3.95 (s, 2H), 3.53 (t, J = 5.6 |
| | | | Hz, 2H), 2.68 (t, J = 5.6 Hz, |
| | | | 2H). |
| | | | MS (ESI) m/z 285.1 |
| | | | [C₁₆H₁₆N₂OS + H]⁺. |
| 116 | J | 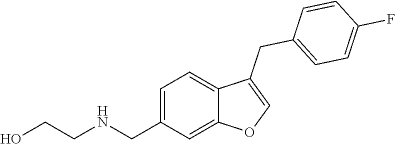 [SMILES: OCCNCc1ccc2c(Cc3ccc(F)cc3)coc2c1] | ¹H NMR (400 MHz, DMSO-d₆) δ (ppm): 8.24 (s, 1H; HCOOH), 7.82 (s, 1H), 7.55 (s, 1H), 7.41 (d, J = 7.6 Hz, 1H), 7.36-7.33 (m, 2H), 7.20 (dd, J = 8.0 Hz, 1.2 Hz, 1H), 7.13-7.08 (m, 2H), 4.00 (s, 2H), 3.89 (s, 2H), 3.50 (t, J = 5.6 Hz, 2H), 2.63 |
| | | | (t, J = 5.6 Hz, 2H). |
| | | | MS (ESI) m/z 300.2 |
| | | | [C₁₈H₁₈FNO₂ + H]⁺. |
| 118 | A | 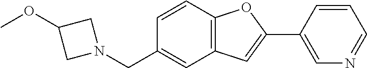 [SMILES: COC1CN(Cc2ccc3oc(-c4cccnc4)cc3c2)C1] | ¹H NMR (400 MHz, DMSO-d₆) δ (ppm): 9.13 (d, J = 1.6 Hz, 1H), 8.59 (dd, J = 4.8 Hz, 1.6 Hz, 1H), 8.27 (dt, J = 8.0 Hz, 1.6 Hz, 1H), |
| | | | 8.16 (s, 1H; HCOOH), |
| | | | 7.59-7.52 (m, 4H), 7.27- |
| | | | 7.25 (m, 1H), 3.99-3.96 (m, |
| | | | 1H), 3.67 (s, 2H), 3.48 (dt, J = |
| | | | 6.0 Hz, 2.0 Hz, 2H), 3.15 |
| | | | (s, 3H), 2.87 (dt, J = 6.0 Hz, |
| | | | 2.0 Hz, 2H). |
| | | | MS (ESI) m/z 295.3 |
| | | | [C₁₈H₁₈N₂O₂ + H]⁺. |

TABLE 5
Summary of compounds (series II) prepared according to the examples above and characterizations thereof

| Cpd | Synthesis | | NMR and Mass |
|---|---|---|---|
| No. | Route | Structure | Spectrometry |
| 81 | A | 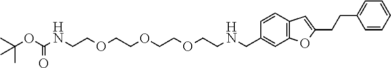 [SMILES: CC(C)(C)OC(=O)NCCOCCOCCOCCNCc1ccc2cc(CCc3ccccc3)oc2c1] | ¹H NMR (400 MHz, DMSO- d₆) δ (ppm): 7.46-7.42 (m, 2H), 7.28-7.25 (m, 4H), 7.20-7.14 (m, 2H), 6.78 (t, 1H), 6.52 (s, 1H), 3.82 (s, |
| | | | 2H), 3.50-3.45 (m, 10H), |
| | | | 3.35-3.34 (m, 2H), 3.10-2.99 |
| | | | (m, 6H), 2.68-2.67 (m, 2H), |
| | | | 1.36 (s, 9H). |
| | | | MS (ESI) m/z 527.4 |
| | | | [C₃₀H₄₂N₂O₆ + H]⁺. |
| 82 | A | 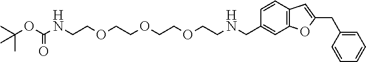 [SMILES: CC(C)(C)OC(=O)NCCOCCOCCOCCNCc1ccc2cc(Cc3ccccc3)oc2c1] | ¹H NMR (400 MHz, DMSO- d₆) δ (ppm): 7.45 (d, J = 8.0 Hz, 1H), 7.41 (s, 1H), 7.35- 7.30 (m, 4H), 7.27-7.23 (m, 1H), 7.14 (dd, J = 8.0 Hz, 1.2 |
| | | | Hz, 1H), 6.77 (t, J = 5.2 Hz, |
| | | | 1H), 6.55 (d, J = 0.8 Hz, 1H), |
| | | | 4.12 (s, 2H), 3.77 (s, 2H), |
| | | | 3.49-3.44 (m, 10H), 3.36- |
| | | | 3.34 (m, 2H), 3.04 (q, J = 5.6 |
| | | | Hz, 2H), 2.61 (t, J = 5.6 Hz, |
| | | | 2H), 1.36 (s, 9H). |
| | | | MS (ESI) m/z 513.4 |
| | | | [C₂₉H₄₀N₂O₆ + H]⁺. |
| 83 | A | 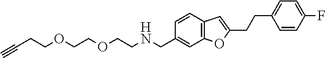 [SMILES: C#CCCOCCOCCNCc1ccc2cc(CCc3ccc(F)cc3)oc2c1] | ¹H NMR (400 MHz, DMSO- d₆) δ (ppm): 7.43-7.41 (m, 2H), 7.31-7.27 (m, 2H), 7.15-7.07 (m, 3H), 6.51 (s, 1H), 3.78 (s, 2H), 3.53-3.46 |
| | | | (m, 8H), 3.08-2.99 (m, 4H), |
| | | | 2.78 (t, J = 2.4 Hz, 1H), 2.63 |
| | | | (t, J = 6.0 Hz, 2H), 2.39-2.37 |
| | | | (m, 2H), 2.12 (br. s, 1H). |
| | | | MS (ESI) m/z 410.2 |
| | | | [C₂₅H₂₈FNO₃ + H]⁺. |
| 84 | A | 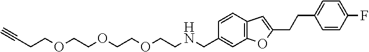 [SMILES: C#CCCOCCOCCOCCNCc1ccc2cc(CCc3ccc(F)cc3)oc2c1] | ¹H NMR (400 MHz, DMSO- d₆) δ (ppm): 7.43-7.41 (m, 2H), 7.31-7.27 (m, 2H), 7.15- 7.07 (m, 3H), 6.51 (s, 1H), |
| | | | 3.78 (s, 2H), 3.52-3.45 (m, |
| | | | 12H), 3.09-2.98 (m, 4H), 2.78 |
| | | | (t, J = 2.8 Hz, 1H), 2.63 (t, |
| | | | J = 5.6 Hz, 2H), 2.39-2.33 (m, |
| | | | 2H), 2.17 (br, 1H). |
| | | | MS (ESI) m/z 454.1 |
| | | | [C₂₇H₃₂FNO₄ + H]⁺. |
| 85 | A | 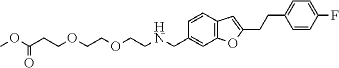 [SMILES: COC(=O)CCOCCOCCNCc1ccc2cc(CCc3ccc(F)cc3)oc2c1] | ¹H NMR (400 MHz, DMSO- d₆) δ (ppm): 7.43-7.41 (m, 2H), 7.31-7.27 (m, 2H), 7.15- 7.07 (m, 3H), 6.51 (s, 1H), 3.78 (s, 2H), 3.62 (t, J = 6.0 |
| | | | Hz, 2H), 3.57 (s, 3H), 3.49- |
| | | | 3.44 (m, 6H), 3.06-3.00 (m, |
| | | | 4H), 2.62 (t, J = 6.0 Hz, 2H), |
| | | | 2.54-2.52 (m, 2H). |
| | | | MS (ESI) m/z 444.2 |
| | | | [C₂₅H₃₀FNO₅ + H]⁺. |
| 86 | A | 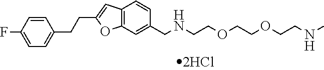 [SMILES: CNCCOCCOCCNCc1ccc2cc(CCc3ccc(F)cc3)oc2c1] | ¹H NMR (400 MHz, DMSO- d₆) δ ppm: 9.26 (br, 2H), 8.76 (br, 2H), 7.76 (s, 1H), 7.56 (d, J = 8.0 Hz, 1H), 7.36 (dd, J = 8.0 Hz, 1.2 Hz, 1H), 7.31- |
| | | | 7.28 (m, 2H), 7.10 (t, J = 8.8 |
| | | | Hz, 2H), 6.61 (s, 1H), 4.25 (s, |
| | | | 2H), 3.72 (t, J = 5.2 Hz, 2H), |
| | | | 3.68 (t, J = 5.2 Hz, 2H), 3.60 |
| | | | (s, 4H), 3.12-3.00 (m, 8H), |
| | | | 2.54 (s, 3H). |
| | | | MS (ESI) m/z 415.2 |
| | | | [C₂₄H₃₁FN₂O₃ + H]⁺. |
| 87 | A | 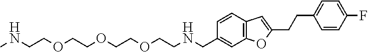 [SMILES: CNCCOCCOCCOCCNCc1ccc2cc(CCc3ccc(F)cc3)oc2c1] | ¹H NMR (400 MHz, DMSO- d₆) δ (ppm): 7.52 (s, 1H), 7.47 (d, J = 7.6 Hz, 1H), 7.31-7.27 (m, 2H), 7.20 (d, J = 8.0 Hz, |
| | | | 1H), 7.12-7.07 (m, 2H), 6.55 |
| | | | (s, 1H), 3.91 (s, 2H), 3.61 (t, |
| | | | J = 5.2 Hz, 2H), 3.55-3.51 (m, |
| | | | 10H), 3.09-3.06 (m, 2H), |
| | | | 3.03-2.98 (m, 4H), 2.75 (t, |
| | | | J = 5.2 Hz, 2H), 2.52 (s, 3H). |
| | | | MS (ESI) m/z 459.2 |
| | | | [C₂₆H₃₅FN₂O₄ + H]⁺. |
| 88 | A | 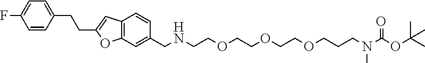 [SMILES: CN(CCCOCCOCCOCCNCc1ccc2cc(CCc3ccc(F)cc3)oc2c1)C(=O)OC(C)(C)C] | ¹H NMR (400 MHz, DMSO- d₆) δ (ppm): 7.43-7.41 (m, 2H), 7.31-7.27 (m, 2H), 7.15-7.07 (m, 3H), 6.51 (s, 1H), 3.78 (s, 2H), 3.50-3.44 |
| | | | (m, 10H), 3.36-3.34 (m, 2H), |
| | | | 3.16 (t, J = 7.2 Hz, 2H), 3.08- |
| | | | 3.04 (m, 2H), 3.02-2.98 (m, |
| | | | 2H), 2.74 (s, 3H), 2.63 (t, J = |
| | | | 5.6 Hz, 2H), 2.07 (br, 1H), |
| | | | 1.66-1.61 (m, 2H), 1.37 (s, |
| | | | 9H). |
| | | | MS (ESI) m/z 573.4 |
| | | | [C₃₂H₄₅FN₂O₆ + H]⁺. |
| 89 | A | 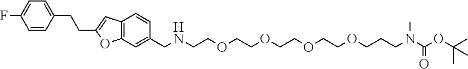 [SMILES: CN(CCCOCCOCCOCCOCCNCc1ccc2cc(CCc3ccc(F)cc3)oc2c1)C(=O)OC(C)(C)C] | ¹H NMR (400 MHz, DMSO- d₆) δ (ppm): 7.43-7.41 (m, 2H), 7.31-7.27 (m, 2H), 7.15-7.12 (m, 1H), 7.11-7.07 (m, 2H), 6.51 (s, 1H), 3.78 (s, |
| | | | 2H), 3.52-3.44 (m, 14H), |
| | | | 3.35-3.33 (m, 2H), 3.17 (t, J = |
| | | | 6.8 Hz, 2H), 3.08-2.99 (m, |
| | | | 4H), 2.74 (s, 3H), 2.63 (t, J = |
| | | | 6.0 Hz, 2H), 2.15 (br, 1H), |
| | | | 1.65 (t, J = 6.4 Hz, 2H), 1.37 |
| | | | (s, 9H). |
| | | | MS (ESI) m/z 617.4 |
| | | | [C₃₄H₄₉FN₂O₇ + H]⁺. |
| 90 | A | 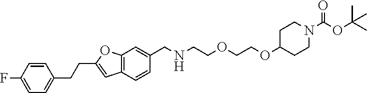 [SMILES: CC(C)(C)OC(=O)N1CCC(OCCOCCNCc2ccc3cc(CCc4ccc(F)cc4)oc3c2)CC1] | ¹H NMR (400 MHz, DMSO- d₆) δ (ppm): 7.43-7.41 (m, 2H), 7.31-7.27 (m, 2H), 7.14- 7.07 (m, 3H), 6.51 (s, 1H), 3.78 (s, 2H), 3.62-3.56 (m, 2H), 3.52-3.39 (m, 7H), 3.08- |
| | | | 2.96 (m, 6H), 2.63 (t, J = 5.6 |
| | | | Hz, 2H), 2.08 (br, 1H), 1.76- |
| | | | 1.72 (m, 2H), 1.37 (s, 9H), |
| | | | 1.30-1.26 (m, 2H). |
| | | | MS (ESI) m/z 541.4 |
| | | | [C₃₁H₄₁FN₂O₅ + H]⁺. |
| 91 | A | 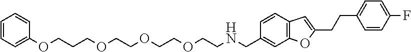 [SMILES: Fc1ccc(CCc2cc3ccc(CNCCOCCOCCOCCCOc4ccccc4)cc3o2)cc1] | ¹H NMR (400 MHz, DMSO- d₆) δ (ppm): 7.43-7.41 (m, 2H), 7.30-7.23 (m, 4H), 7.14-7.12 (m, 1H), 7.11-7.06 |
| | | | (m, 2H), 6.92-6.89 (m, 3H), |
| | | | 6.51 (s, 1H), 3.98 (t, J = 6.4 |
| | | | Hz, 2H), 3.77 (s, 2H), 3.53- |
| | | | 3.45 (m, 12H), 3.08-2.97 (m, |
| | | | 4H), 2.63 (t, J = 5.6 Hz, 2H), |
| | | | 1.94-1.88 (m, 2H). |
| | | | MS (ESI) m/z 536.3 |
| | | | [C₃₂H₃₈FNO₅ + H]⁺. |
| 92 | A | 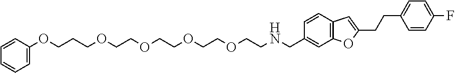 [SMILES: Fc1ccc(CCc2cc3ccc(CNCCOCCOCCOCCOCCCOc4ccccc4)cc3o2)cc1] | ¹H NMR (400 MHz, DMSO- d₆) δ (ppm): 7.46-7.43 (m, 2H), 7.30-7.24 (m, 4H), 7.15 (d, J = 7.6 Hz, 1H), 7.11-7.06 (m, 2H), 6.92-6.89 (m, 3H), |
| | | | 6.52 (s, 1H), 3.98 (t, J = 6.4 |
| | | | Hz, 2H), 3.83 (s, 2H), 3.53- |
| | | | 3.48 (m, 16H), 3.06-3.00 (m, |
| | | | 4H), 2.69-2.67 (m, 2H), |
| | | | 1.93-1.90 (m, 2H). |
| | | | MS (ESI) m/z 580.4 |
| | | | [C₃₄H₄₂FNO₆ + H]⁺. |
| 93 | A | 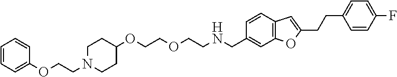 [SMILES: Fc1ccc(CCc2cc3ccc(CNCCOCCOC4CCN(CCOc5ccccc5)CC4)cc3o2)cc1] | ¹H NMR (400 MHz, DMSO- d₆) δ (ppm): 7.46 (s, 1H), 7.43 (d, J = 8.0 Hz, 1H), 7.30-7.25 (m, 4H), 7.15 (d, J = 7.6 Hz, 1H), 7.07 (t, J = 7.2 Hz, 2H), |
| | | | 6.93-6.90 (m, 3H), 6.51 (s, |
| | | | 1H), 4.01 (t, J = 6.0 Hz, 2H), |
| | | | 3.82 (s, 2H), 3.52-3.45 (m, |
| | | | 6H), 3.31-3.22 (m, 1H), 3.07- |
| | | | 2.98 (m, 4H), 2.75-2.72 (m, |
| | | | 2H), 2.67 (t, J = 5.6 Hz, 2H), |
| | | | 2.63 (t, J = 6.0 Hz, 2H), 2.11 |
| | | | (t, J = 9.6 Hz, 2H), 1.80-1.77 |
| | | | (m, 2H), 1.43-1.35 (m, 2H). |
| | | | MS (ESI) m/z 561.4 |
| | | | [C₃₄H₄₁FN₂O₄ + H]⁺. |
| 94 | A | 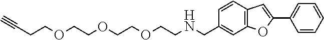 [SMILES: C#CCCOCCOCCOCCNCc1ccc2cc(-c3ccccc3)oc2c1] | ¹H NMR (400 MHz, DMSO- d₆) δ (ppm): 7.92 (d, J = 8.4 Hz, 2H), 7.59-7.56 (m, 2H), |
| | | | 7.52 (t, J = 8.0 Hz, 2H), 7.42- |
| | | | 7.38 (m, 2H), 7.23 (dd, J = 8.0 |
| | | | Hz, 0.8 Hz, 1H), 3.83 (s, 2H), |
| | | | 3.53-3.45 (m, 12H), 2.78 (t, J = |
| | | | 2.8 Hz, 1H), 2.66 (t, J = 5.6 |
| | | | Hz, 2H), 2.39-2.33 (m, 2H). |
| | | | MS (ESI) m/z 408.2 |
| | | | [C₂₅H₂₉NO₄ + H]⁺. |
| 95 | A | 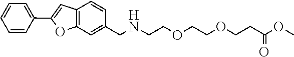 [SMILES: COC(=O)CCOCCOCCNCc1ccc2cc(-c3ccccc3)oc2c1] | ¹H NMR (400 MHz, DMSO- d₆) δ (ppm): 7.92-7.89 (m, 2H), 7.58-7.56 (m, 2H), 7.51 (t, J = 7.6 Hz, 2H), 7.42-7.38 |
| | | | (m, 2H), 7.23 (d, J = 8.8 Hz, |
| | | | 1H), 3.83 (s, 2H), 3.62 (t, J = |
| | | | 6.4 Hz, 2H), 3.57 (s, 3H), |
| | | | 3.50-3.46 (m, 6H), 2.65 (t, J = |
| | | | 5.6 Hz, 2H), 2.54-2.49 (m, |
| | | | 2H), 2.22 (br. s, 1H). |
| | | | MS (ESI) m/z 398.1 |
| | | | [C₂₃H₂₇NO₅ + H]⁺. |
| 96 | A | 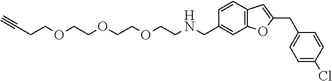 [SMILES: C#CCCOCCOCCOCCNCc1ccc2cc(Cc3ccc(Cl)cc3)oc2c1] | ¹H NMR (400 MHz, DMSO- d₆) δ (ppm): 7.45 (d, J = 8.0 Hz, 1H), 7.42-7.38 (m, 3H), 7.33 (d, J = 8.8 Hz, 2H), 7.15 (d, J = 8.4 Hz, 1H), 6.57 (s, 1H), 4.14 (s, 2H), 3.78 (s, |
| | | | 2H), 3.51-3.44 (m, 12H), 2.77 |
| | | | (t, J = 2.8 Hz, 1H), 2.62 (t, J = |
| | | | 5.6 Hz, 2H), 2.38-2.34 (m, |
| | | | 2H). |
| | | | MS (ESI) m/z 456.1 |
| | | | [C₂₆H₃₀ClNO₄ + H]⁺. |
| 97 | A | 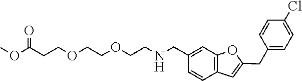 [SMILES: COC(=O)CCOCCOCCNCc1ccc2cc(Cc3ccc(Cl)cc3)oc2c1] | ¹H NMR (400 MHz, DMSO- d₆) δ (ppm): 7.47 (d, J = 8.0 Hz, 1H), 7.44 ( s, 1H), 7.40- 7.37 (m, 2H), 7.34-7.32 (m, 2H), 7.17 (d, J = 8.0, 1H), |
| | | | 6.58 (s, 1H), 4.14 (s, 2H), |
| | | | 3.82 (s, 2H), 3.61 (t, J = 6.4 |
| | | | Hz, 2H), 3.56 (s, 3H), 3.50- |
| | | | 3.45 (m, 6H), 2.66 (t, J = 5.6 |
| | | | Hz, 2H), 2.53-2.49 (m, 2H). |
| | | | MS (ESI) m/z 446.1 |
| | | | [C₂₄H₂₈ClNO₅ + H]⁺. |
| 98 | B | 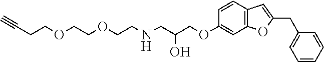 [SMILES: C#CCCOCCOCCNCC(O)COc1ccc2cc(Cc3ccccc3)oc2c1] | ¹H NMR (400 MHz, DMSO- d₆) δ (ppm): 7.39 (d, J = 8.4 Hz, 1H), 7.35-7.29 (m, 4H), 7.26-7.22 (m, 1H), 7.09 (d, J = |
| | | | 2.0 Hz, 1H), 6.81 (dd, J = |
| | | | 8.4 Hz, 2.0 Hz, 1H), 6.50 (s, |
| | | | 1H), 4.98 (d, J = 4.0 Hz, 1H), |
| | | | 4.09 (s, 2H), 3.94-3.91 (m, |
| | | | 1H), 3.88-3.85 (m, 2H), |
| | | | 3.53-3.44 (m, 8H), 2.78 (t, J = |
| | | | 2.8 Hz, 1H), 2.68-2.65 (m, |
| | | | 2H), 2.59-2.55 (m, 2H), |
| | | | 2.39-2.33 (m, 2H), 1.8 (br. s, |
| | | | 1H). |
| | | | MS (ESI) m/z 438.2 |
| | | | [C₂₆H₃₁NO₅ + H]⁺. |
| 99 | B | 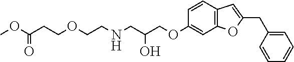 [SMILES: COC(=O)CCOCCNCC(O)COc1ccc2cc(Cc3ccccc3)oc2c1] | ¹H NMR (400 MHz, DMSO- d₆) δ (ppm): 7.39 (d, J = 8.4 Hz, 1H), 7.35-7.29 (m, 4H), 7.26-7.22 (m, 1H), 7.09 (d, |
| | | | J = 1.6 Hz, 1H), 6.81 (dd, J = |
| | | | 8.4 Hz, 2.0 Hz, 1H), 6.50 (s, |
| | | | 1H), 4.98 (br. s, 1H), 4.09 (s, |
| | | | 2H), 3.96-3.91 (m, 1H), |
| | | | 3.88-3.85 (m, 2H), 3.62-3.58 |
| | | | (m, 2H), 3.56 (s, 3H), 3.43 (t, |
| | | | J = 5.6 Hz, 2H) 2.68-2.63 (m, |
| | | | 3H), 2.56-2.50 (m, 3H), 1.89 |
| | | | (br. s, 1H). |
| | | | MS (ESI) m/z 428.2 |
| | | | [C₂₄H₂₉NO₆ + H]⁺. |
| 120 | A | 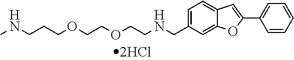 [SMILES: CNCCCOCCOCCNCc1ccc2cc(-c3ccccc3)oc2c1] | ¹H NMR (400 MHz, DMSO- d₆) δ (ppm): 9.35 (br. s, 2H), 8.75 (br. s, 2H), 7.96 (d, J = 8.4 Hz, 2H), 7.89 (s, 1H), 7.72 |
| | | | (d, J = 8.0 Hz, 1H), 7.55-7.49 |
| | | | (m, 3H), 7.47-7.42 (m, 2H), |
| | | | 4.30 (br. s, 2H), 3.73 (t, J = |
| | | | 5.2 Hz, 2H), 3.59-3.55 (m, |
| | | | 4H), 3.48 (t, J = 6.0 Hz, 2H), |
| | | | 3.08 (br. s, 2H), 2.90 (br. s, |
| | | | 2H), 2.49 (s, 3H), 1.87-1.81 |
| | | | (m, 2H). |
| | | | MS (ESI) m/z 383.2 |
| | | | [C₂₃H₃₀N₂O₃ + H]⁺. |
| 121 | A | 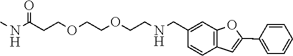 [SMILES: CNC(=O)CCOCCOCCNCc1ccc2cc(-c3ccccc3)oc2c1] | ¹H NMR (400 MHz, DMSO- d₆) δ (ppm): 7.92-7.90 (m, 2H), 7.75 (br. s, 1H), 7.58- 7.56 (m, 2H), 7.51 (t, J = 7.6 |
| | | | Hz, 2H), 7.42-7.38 (m, 2H), |
| | | | 7.23 (d, J = 8.0 Hz, 1H), 3.83 |
| | | | (s, 2H), 3.58 (t, J = 6.4 Hz, |
| | | | 2H), 3.49-3.46 (m, 6H), 2.65 |
| | | | (t, J = 5.6 Hz, 2H), 2.54 (d, J = |
| | | | 4.4 Hz, 3H), 2.27 (t, J = 6.4 |
| | | | Hz, 2H), 2.21 (br. s, 1H). |
| | | | MS (ESI) m/z 397.2 |
| | | | [C₂₃H₂₈N₂O₄ + H]⁺. |
| 122 | A | 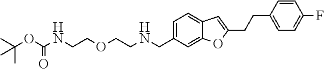 [SMILES: CC(C)(C)OC(=O)NCCOCCNCc1ccc2cc(CCc3ccc(F)cc3)oc2c1] | ¹H NMR (400 MHz, CDCl₃) δ (ppm): 7.43 − 7.37 (m, 2H), 7.19 − 7.10 (m, 3H), 7.01 − 6.91 (m, 2H), 6.31 (s, 1H), 5.02 (s, 1H), 3.91 (s, 2H), |
| | | | 3.58 (t, J = 5.2 Hz, 2H), 3.50 |
| | | | (t, J = 5.2 Hz, 2H), 3.31 (q, J = |
| | | | 5.2 Hz, 2H), 3.03 (s, 4H), |
| | | | 2.82 (t, J = 5.2 Hz, 2H), 1.44 |
| | | | (s, 9H). |
| | | | MS (ESI) m/z 457.7 |
| | | | [C₂₆H₃₃FN₂O₄ + H]⁺. |
| 123 | A | 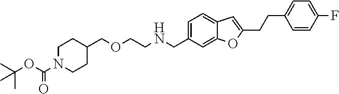 [SMILES: CC(C)(C)OC(=O)N1CCC(COCCNCc2ccc3cc(CCc4ccc(F)cc4)oc3c2)CC1] | ¹H NMR (400 MHz, CDCl₃) δ (ppm): 7.43 − 7.37 (m, 2H), 7.18 − 7.10 (m, 3H), 7.01 − 6.87 (m, 2H), 6.30 (d, J = 0.8 Hz, 1H), 4.09 (s, 2H), 3.90 (s, 2H), 3.55 (dd, J = 5.6 Hz, 4.8 |
| | | | Hz, 2H), 3.27 (d, J = 6.0 Hz, |
| | | | 2H), 3.03 (s, 4H), 2.81 (t, J = |
| | | | 5.2 Hz, 2H), 2.74 − 2.63 (m, |
| | | | 2H), 1.77 − 1.64 (m, 3H), 1.45 |
| | | | (s, 9H), 1.19 − 1.05 (m, 2H). |
| | | | MS (ESI) m/z 511.9 |
| | | | [C₃₀H₃₉FN₂O₄ + H]⁺. |
| 124 | A | 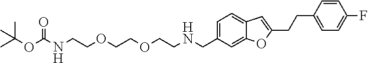 [SMILES: CC(C)(C)OC(=O)NCCOCCOCCNCc1ccc2cc(CCc3ccc(F)cc3)oc2c1] | ¹H NMR (400 MHz, CDCl₃) δ (ppm): 7.39 (d, J = 8.0 Hz, 2H), 7.18 − 7.10 (m, 3H), 7.00 − 6.88 (m, 2H), 6.30 (s, 1H), 5.18 (s, 1H), 3.90 (s, |
| | | | 2H), 3.62 (t, J = 5.2 Hz, 2H), |
| | | | 3.60 (s, 4H), 3.53 (t, J = 5.2 |
| | | | Hz, 2H), 3.30 (q, J = 5.2 Hz, |
| | | | 2H), 3.03 (s, 4H), 2.83 (t, J = |
| | | | 5.2 Hz, 2H), 1.43 (s, 9H). |
| | | | MS (ESI) m/z 501.6 |
| | | | [C₂₈H₃₇FN₂O₅ + H]⁺. |
| 125 | A | 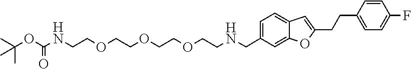 [SMILES: CC(C)(C)OC(=O)NCCOCCOCCOCCNCc1ccc2cc(CCc3ccc(F)cc3)oc2c1] | ¹H NMR (400 MHz, CDCl₃) δ (ppm): 7.43 − 7.36 (m, 2H), 7.18 − 7.10 (m, 3H), 7.00 − 6.89 (m, 2H), 6.30 (s, 1H), 5.29 (s, 1H), 3.90 (s, 2H), |
| | | | 3.68 − 3.55 (m, 10H), 3.50 (t, |
| | | | J = 5.2 Hz, 2H), 3.29 (q, J = |
| | | | 5.2 Hz, 2H), 3.03 (s, 4H), |
| | | | 2.83 (t, J = 5.2 Hz, 2H), 1.43 |
| | | | (s, 9H). |
| | | | MS (ESI) m/z 545.7 |
| | | | [C₃₀H₄₁FN₂O₆ + H]⁺. |
| 126 | A | 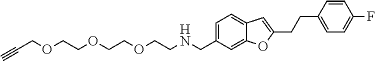 [SMILES: C#CCOCCOCCOCCNCc1ccc2cc(CCc3ccc(F)cc3)oc2c1] | ¹H NMR (400 MHz, CDCl₃) δ (ppm): 7.47 − 7.36 (m, 2H), 7.18 − 7.10 (m, 3H), 7.01 − 6.90 (m, 2H), 6.30 (s, 1H), |
| | | | 4.18 (d, J = 2.4 Hz, 2H), 3.90 |
| | | | (s, 2H), 3.73 − 3.59 (m, 10H), |
| | | | 3.03 (s, 4H), 2.83 (t, J = 5.2 |
| | | | Hz, 2H), 2.41 (t, J = 2.4 Hz, |
| | | | 1H). |
| | | | MS (ESI) m/z 440.5 |
| | | | [C₂₆H₃₀FNO₄ + H]⁺. |
| 127 | A | 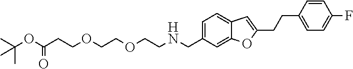 [SMILES: CC(C)(C)OC(=O)CCOCCOCCNCc1ccc2cc(CCc3ccc(F)cc3)oc2c1] | ¹H NMR (400 MHz, CDCl₃) δ (ppm): 7.43 − 7.36 (m, 2H), 7.18 − 7.09 (m, 3H), 7.01 − 6.90 (m, 2H), 6.30 (s, 1H), 3.90 (s, 2H), 3.71 (t, J = 6.4 |
| | | | Hz, 2H), 3.65 − 3.57 (m, 6H), |
| | | | 3.03 (s, 4H), 2.82 (t, J = 5.2 |
| | | | Hz, 2H), 2.50 (t, J = 6.4 Hz, |
| | | | 2H), 1.44 (s, 9H). |
| | | | MS (ESI) m/z 486.8 |
| | | | [C₂₈H₃₆FNO₅ + H]⁺. |
| 128 | A | 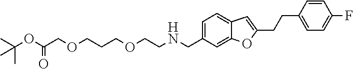 [SMILES: CC(C)(C)OC(=O)COCCCOCCNCc1ccc2cc(CCc3ccc(F)cc3)oc2c1] | ¹H NMR (400 MHz, CDCl₃) δ (ppm): 7.40 (d, J = 8.0 Hz, 2H), 7.18 − 7.10 (m, 3H), 7.01 − 6.90 (m, 2H), 6.30 (s, 1H), 3.94 (s, 2H), 3.91 (s, 2H), |
| | | | 3.63 − 3.51 (m, 6H), 3.03 (s, |
| | | | 4H), 2.81 (t, J = 5.2 Hz, 2H), |
| | | | 1.89 (p, J = 6.4 Hz, 2H), 1.47 |
| | | | (s, 9H). |
| | | | MS (ESI) m/z 486.8 |
| | | | [C₂₈H₃₆FNO₅ + H]⁺. |
| 129 | A | 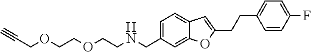 [SMILES: C#CCOCCOCCNCc1ccc2cc(CCc3ccc(F)cc3)oc2c1] | ¹H NMR (400 MHz, CDCl₃) δ (ppm): 7.43 − 7.36 (m, 2H), 7.19 − 7.09 (m, 3H), 7.01 − 6.90 (m, 2H), 6.30 (d, J = 0.8 |
| | | | Hz, 1H), 4.20 (d, J = 2.4 Hz, |
| | | | 2H), 3.90 (s, 2H), 3.75 − 3.58 |
| | | | (m, 6H), 3.03 (s, 4H), 2.84 (t, |
| | | | J = 5.2 Hz, 2H), 2.42 (t, J = |
| | | | 2.4 Hz, 1H). |
| | | | MS (ESI) m/z 396.5 |
| | | | [C₂₄H₂₆FNO₃ + H]⁺. |
| 130 | A | 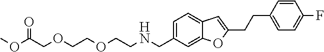 [SMILES: COC(=O)COCCOCCNCc1ccc2cc(CCc3ccc(F)cc3)oc2c1] | ¹H NMR (400 MHz, CDCl₃) δ (ppm): 7.67 (s, 1H), 7.45 (d, J = 8.0 Hz, 1H), 7.36 (dd, J = 8.0, 1.6 Hz, 1H), 7.16 − 7.07 |
| | | | (m, 2H), 7.00 − 6.90 (m, 2H), |
| | | | 6.30 (s, 1H), 4.29 (s, 2H), |
| | | | 4.12 (s, 2H), 3.86 (t, J = 4.8 |
| | | | Hz, 2H), 3.72 − 3.66 (m, 7H), |
| | | | 3.09 (t, J = 4.8 Hz, 2H), 3.00 |
| | | | (s, 4H). |
| | | | MS (ESI) m/z 430.5 |
| | | | [C₂₄H₂₈FNO₅ + H]⁺. |
| 131 | A | 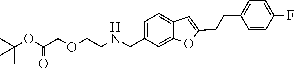 [SMILES: CC(C)(C)OC(=O)COCCNCc1ccc2cc(CCc3ccc(F)cc3)oc2c1] | ¹H NMR (400 MHz, DMSO- d₆) δ (ppm): 7.42 (d, J = 8.4 Hz, 2H), 7.32 − 7.24 (m, 2H), 7.17 − 7.03 (m, 3H), 6.51 (s, 1H), 3.96 (s, 2H), 3.78 (s, |
| | | | 2H), 3.52 (t, J = 5.6 Hz, 2H), |
| | | | 3.10 − 2.95 (m, 4H), 2.64 (t, |
| | | | J = 5.6 Hz, 2H), 1.41 (s, 9H). |
| | | | MS (ESI) m/z 428.5 |
| | | | [C₂₅H₃₀FNO₄ + H]⁺. |
| 132 | A | 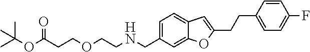 [SMILES: CC(C)(C)OC(=O)CCOCCNCc1ccc2cc(CCc3ccc(F)cc3)oc2c1] | ¹H NMR (400 MHz, CDCl₃) δ (ppm): 7.44 − 7.36 (m, 2H), 7.19 − 7.09 (m, 3H), 7.01 − 6.90 (m, 2H), 6.30 (d, J = 0.8 |
| | | | Hz, 1H), 3.91 (s, 2H), 3.68 (t, |
| | | | J = 6.4 Hz, 2H), 3.63 − 3.56 |
| | | | (m, 2H), 3.03 (s, 4H), 2.82 (t, |
| | | | J = 5.2 Hz, 2H), 2.48 (t, J = |
| | | | 6.4 Hz, 2H), 1.43 (s, 9H). |
| | | | MS (ESI) m/z 442.5 |
| | | | [C₂₆H₃₂FNO₄ + H]⁺. |
| 133 | A | 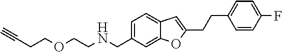 [SMILES: C#CCCOCCNCc1ccc2cc(CCc3ccc(F)cc3)oc2c1] | ¹H NMR (400 MHz, CDCl₃) δ (ppm): 7.44 − 7.37 (m, 2H), 7.19 − 7.09 (m, 3H), 7.01 − 6.91 (m, 2H), 6.30 (d, J = 0.8 |
| | | | Hz, 1H), 3.91 (s, 2H), 3.67 − |
| | | | 3.54 (m, 4H), 3.03 (s, 4H), |
| | | | 2.87 − 2.80 (m, 2H), 2.47 (td, |
| | | | J = 6.8 Hz, 2.8 Hz, 2H), 1.97 |
| | | | (t, J = 2.8 Hz, 1H). |
| | | | MS (ESI) m/z 366.5 |
| | | | [C₂₃H₂₄FNO₂ + H]⁺. |
| 134 | A | 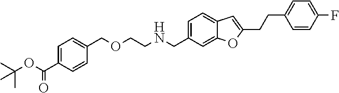 [SMILES: CC(C)(C)OC(=O)c1ccc(COCCNCc2ccc3cc(CCc4ccc(F)cc4)oc3c2)cc1] | ¹H NMR (400 MHz, CDCl₃) δ (ppm): 8.00 − 7.93 (m, 2H), 7.45 − 7.33 (m, 4H), 7.19 − 7.09 (m, 3H), 7.01 − 6.91 (m, 2H), 6.30 (d, J = 0.8 Hz, 1H), 4.57 (s, 2H), 3.94 (s, 2H), |
| | | | 3.65 (t, J = 5.2 Hz, 2H), 3.03 |
| | | | (s, 4H), 2.89 (t, J = 5.2 Hz, |
| | | | 2H), 1.59 (s, 9H). |
| | | | MS (ESI) m/z 504.7 |
| | | | [C₃₁H₃₄FNO₄ + H]⁺. |
| 135 | A | 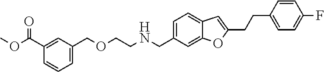 [SMILES: COC(=O)c1cccc(COCCNCc2ccc3cc(CCc4ccc(F)cc4)oc3c2)c1] | ¹H NMR (400 MHz, CDCl₃) δ (ppm): 8.00 (s, 1H), 7.96 (d, J = 7.6 Hz, 1H), 7.54 (d, J = 7.6 Hz, 1H), 7.46 − 7.37 (m, 3H), 7.20 − 7.10 (m, 3H), 6.96 |
| | | | (t, J = 8.4 Hz, 2H), 6.30 (s, |
| | | | 2H), 3.91 (s, 3H), 3.67 (t, J = |
| | | | 5.2 Hz, 2H), 3.03 (s, 4H), 2.90 |
| | | | (t, J = 5.2 Hz, 2H). |
| | | | MS (ESI) m/z 462.6 |
| | | | [C₂₈H₂₈FNO₄ + H]⁺. |
| 136 | A | 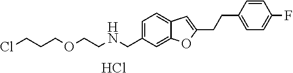 [SMILES: Fc1ccc(CCc2cc3ccc(CNCCOCCCCl)cc3o2)cc1 ; Cl] | ¹H NMR (400 MHz, DMSO- d₆) δ (ppm): 9.19 (s, 2H; HC1), 7.73 (s, 1H), 7.57 (d, J = 8.0 Hz, 1H), 7.35 (dd, J = 8.0 Hz, 1.6 Hz, 1H), 7.34 − |
| | | | 7.24 (m, 2H), 7.14 − 7.04 (m, |
| | | | 2H), 6.60 (d, J = 0.8 Hz, 1H), |
| | | | 4.24 (s, 2H), 3.73 (t, J = 6.4 |
| | | | Hz, 2H), 3.66 (t, J = 5.2 Hz, |
| | | | 2H), 3.53 (t, J = 6.0 Hz, 2H), |
| | | | 3.14 − 2.98 (m, 6H), 1.97 (p, |
| | | | J = 6.4 Hz, 2H). |
| | | | MS (ESI) m/z 390.6 |
| | | | [C₂₂H₂₅CIFNO₂ + H]⁺. |
| 137 | A | 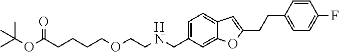 [SMILES: CC(C)(C)OC(=O)CCCCOCCNCc1ccc2cc(CCc3ccc(F)cc3)oc2c1] | ¹H NMR (400 MHz, CDCl₃) δ (ppm): 7.49 (s, 1H), 7.42 (d, J = 8.0 Hz, 1H), 7.22 (dd, J = 8.0 Hz, 1.6 Hz, 1H), 7.19 − |
| | | | 7.10 (m, 2H), 7.01 − 6.91 (m, |
| | | | 2H), 6.31 (s, 1H), 4.02 (s, |
| | | | 2H), 3.62 (t, J = 5.2 Hz, 2H), |
| | | | 3.45 (t, J = 6.0 Hz, 2H), 3.03 |
| | | | (s, 4H), 2.89 (t, J = 5.2 Hz, |
| | | | 2H), 2.25 (t, J = 7.2 Hz, 2H), |
| | | | 1.71 − 1.55 (m, 4H), 1.43 (s, 9H). |
| | | | MS (ESI) m/z 470.6 |
| | | | [C₂₈H₃₆FNO₄ + H]⁺. |
| 138 | A | 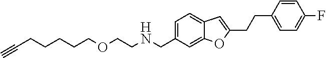 [SMILES: C#CCCCCCOCCNCc1ccc2cc(CCc3ccc(F)cc3)oc2c1] | ¹H NMR (400 MHz, DMSO- d₆) δ (ppm): 7.42 (d, J = 8.0 Hz, 2H), 7.34 − 7.24 (m, 2H), 7.17 − 7.03 (m, 3H), 6.51 (d, |
| | | | J = 0.8 Hz, 1H), 3.78 (s, 2H), |
| | | | 3.42 (t, J = 5.6 Hz, 2H), 3.34 |
| | | | (s, 4H), 3.10 − 2.95 (m, 4H), |
| | | | 2.71 (t, J = 2.8 Hz, 1H), 2.62 |
| | | | (t, J = 5.6 Hz, 2H), 2.13 (td, J = |
| | | | 6.8 Hz, 2.8 Hz, 2H), 1.54 − |
| | | | 1.30 (m, 4H). |
| | | | MS (ESI) m/z 408.5 |
| | | | [C₂₆H₃₀FNO₂ + H]⁺. |
| 139 | A | 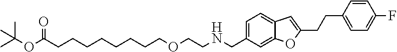 [SMILES: CC(C)(C)OC(=O)CCCCCCCCOCCNCc1ccc2cc(CCc3ccc(F)cc3)oc2c1] | ¹H NMR (400 MHz, CDCl₃) δ (ppm): 7.45 − 7.33 (m, 2H), 7.20 − 7.10 (m, 3H), 7.00 − 6.91 (m, 2H), 6.30 (d, J = 0.8 |
| | | | Hz, 1H), 3.94 (s, 2H), 3.57 (t, |
| | | | J = 5.2 Hz, 2H), 3.42 (t, J = |
| | | | 6.8 Hz, 2H), 3.03 (s, 4H), 2.85 |
| | | | (t, J = 5.2 Hz, 2H), 2.19 (t, J = |
| | | | 7.6 Hz, 2H), 1.61 − 1.52 (m, |
| | | | 1H), 1.43 (s, 9H), 1.29 (d, J = |
| | | | 5.2 Hz, 11H). |
| | | | MS (ESI) m/z 526.7 |
| | | | [C32H44FNO4 + H]⁺. |
| 140 | A | 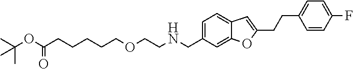 [SMILES: CC(C)(C)OC(=O)CCCCCOCCNCc1ccc2cc(CCc3ccc(F)cc3)oc2c1] | ¹H NMR (400 MHz, CDCl₃) δ (ppm): 7.46 (s, 1H), 7.42 (d, J = 8.0 Hz, 1H), 7.22 − 7.10 (m, 3H), 6.96 (t, J = 8.8 Hz, 2H), 6.31 (s, 1H), 4.00 (s, |
| | | | 2H), 3.60 (t, J = 5.2 Hz, 2H), |
| | | | 3.43 (t, J = 6.4 Hz, 2H), 3.03 |
| | | | (s, 4H), 2.88 (t, J = 5.2 Hz, |
| | | | 2H), 2.21 (t, J = 7.6 Hz, 2H), |
| | | | 1.65 − 1.52 (m, 4H), 1.43 (s, |
| | | | 9H), 1.39 − 1.29 (m, 2H). |
| | | | MS (ESI) m/z 484.7 |
| | | | [C₂₉H₃₈FNO₄ + H]⁺. |
| 141 | A | 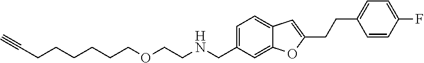 [SMILES: C#CCCCCCCOCCNCc1ccc2cc(CCc3ccc(F)cc3)oc2c1] | ¹H NMR (400 MHz, CDCl₃) δ (ppm): 7.44 − 7.37 (m, 2H), 7.20 − 7.10 (m, 3H), 7.01 − 6.90 (m, 2H), 6.30 (d, J = 0.8 Hz, 1H), 3.92 (s, 2H), 3.56 (t, |
| | | | J = 5.2 Hz, 2H), 3.43 (t, J = |
| | | | 6.8 Hz, 2H), 3.03 (s, 4H), 2.83 |
| | | | (t, J = 5.2 Hz, 2H), 2.18 (td, J = |
| | | | 6.8 Hz, 2.8 Hz, 2H), 1.93 (t, |
| | | | J = 2.8 Hz, 1H), 1.65 − 1.48 |
| | | | (m, 4H), 1.48 − 1.26 (m, 4H). |
| | | | MS (ESI) m/z 422.5 |
| | | | [C₂₇H₃₂FNO₂ + H]⁺. |
| 142 | A | 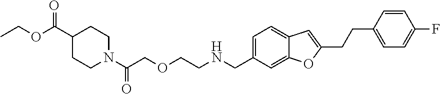 [SMILES: CCOC(=O)C1CCN(C(=O)COCCNCc2ccc3cc(CCc4ccc(F)cc4)oc3c2)CC1] | ¹H NMR (400 MHz, DMSO- d₆) δ (ppm): 7.45 − 7.38 (m, 2H), 7.33 − 7.23 (m, 2H), 7.13 (dd, J = 8.0 Hz, 1.6 Hz, 1H), 7.13 − 7.04 (m, 2H), 6.50 (s, 1H), 4.21 − 4.00 (m, 5H), 3.78 (s, 2H), 3.72 (d, J = 12.8 Hz, |
| | | | 1H), 3.50 (td, J = 6.0 Hz, 1.6 |
| | | | Hz, 2H), 3.10 − 2.95 (m, 5H), |
| | | | 2.72 (t, J = 12.4 Hz, 1H), 2.65 |
| | | | (t, J = 5.6 Hz, 2H), 2.62 − 2.51 |
| | | | (m, 1H), 1.86 − 1.75 (m, 2H), |
| | | | 1.57 − 1.43 (m, 1H), 1.43 − |
| | | | 1.30 (m, 1H), 1.16 (t, J = 7.2 |
| | | | Hz, 3H). |
| | | | MS (ESI) m/z 511.7 |
| | | | [C₂₉H₃₅FN₂O₅ + H]⁺. |
| 143 | A | 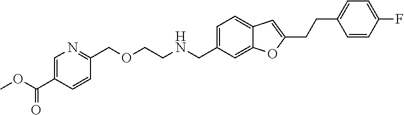 | ¹H NMR (400 MHz, DMSO- d₆) δ (ppm): 9.01 (d, J = 2.0 Hz, 1H), 8.29 (dd, J = 8.4 Hz, 2.0 Hz, 1H), 7.61 (d, J = 8.4 Hz, 1H), 7.47 − 7.38 (m, 2H), 7.28 (dd, J = 8.4 Hz, 5.6 Hz, 2H), 7.14 (d, J = 8.0 Hz, 1H), |
| | | | 7.08 (t, J = 8.8 Hz, 2H), 6.51 |
| | | | (s, 1H), 4.64 (s, 2H), 3.88 (s, |
| | | | 2H), 3.80 (s, 2H), 3.62 (t, J = |
| | | | 5.6 Hz, 2H), 3.03 (dq, J = 13.6 |
| | | | Hz, 6.8 Hz, 4H), 2.73 (t, J = |
| | | | 5.6 Hz, 2H). |
| | | | MS (ESI) m/z 463.7 |
| | | | [C₂₇H2₇FN₂O₄ + H]⁺. |
| 144 | A | 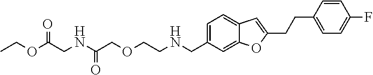 [SMILES: CCOC(=O)CNC(=O)COCCNCc1ccc2cc(CCc3ccc(F)cc3)oc2c1] | ¹H NMR (400 MHz, CDCl₃) δ (ppm): 8.19 (s, 1H), 7.47 − 7.38 (m, 2H), 7.18 (dd, J = 8.0 Hz, 1.2 Hz, 1H), 7.18 − 7.09 (m, 2H), 7.01 − 6.90 (m, 2H), |
| | | | 6.31 (s, 1H), 4.68 (s, 2H), |
| | | | 4.12 (q, J = 7.2 Hz, 2H), 4.02 |
| | | | (s, 4H), 3.70 (t, J = 4.8 Hz, |
| | | | 2H), 3.02 (s, 4H), 2.94 (t, J = |
| | | | 4.8 Hz, 2H), 1.23 (t, J = 7.2 |
| | | | Hz, 3H). |
| | | | MS (ESI) m/z 457.7 |
| | | | [C₂₅H₂₉FN₂O₅ + H]⁺. |
| 145 | A | 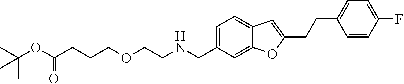 [SMILES: CC(C)(C)OC(=O)CCCOCCNCc1ccc2cc(CCc3ccc(F)cc3)oc2c1] | ¹H NMR (400 MHz, CDCl₃) δ (ppm): 7.40 (d, J = 8.0 Hz, 1H), 7.36 (s, 1H), 7.20 − 7.09 (m, 3H), 7.02 − 6.91 (m, 2H), 6.31 (s, 1H), 3.72 (s, 2H), |
| | | | 3.58 (t, J = 5.2 Hz, 2H), 3.04 |
| | | | (s, 4H), 2.67 (t, J = 5.2 Hz, |
| | | | 2H), 2.54 (t, J = 7.2 Hz, 2H), |
| | | | 2.23 (t, J = 7.2 Hz, 2H), 1.82 |
| | | | (p, J = 7.2 Hz, 2H), 1.42 (s, |
| | | | 9H). |
| | | | MS (ESI) m/z 456.9 |
| | | | [C₂₇H₃₄FNO₄ + H]⁺. |
| 146 | A | 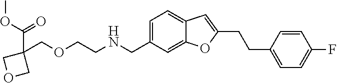 [SMILES: COC(=O)C1(COCCNCc2ccc3cc(CCc4ccc(F)cc4)oc3c2)COC1] | ¹H NMR (400 MHz, CDCl₃) δ (ppm): 7.43 − 7.37 (m, 2H), 7.18 − 7.09 (m, 3H), 7.01 − 6.90 (m, 2H), 6.30 (s, 1H), 4.89 (d, J = 6.4 Hz, 2H), 4.54 (d, J = 6.4 Hz, 2H), 3.90 (d, J = |
| | | | 5.6 Hz, 4H), 3.75 (d, J = 0.8 |
| | | | Hz, 3H), 3.64 (t, J = 5.2 Hz, |
| | | | 2H), 3.03 (s, 4H), 2.82 (t, J = |
| | | | 5.2 Hz, 2H). |
| | | | MS (ESI) m/z 442.5 |
| | | | [C₂₅H₂₈FNO₅ + H]⁺. |
| 147 | A | 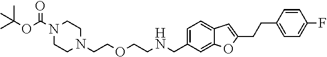 [SMILES: CC(C)(C)OC(=O)N1CCN(CCOCCNCc2ccc3cc(CCc4ccc(F)cc4)oc3c2)CC1] | ¹H NMR (400 MHz, CDCl₃) δ (ppm): 7.46 − 7.38 (m, 2H), 7.20 − 7.10 (m, 3H), 7.01 − 6.89 (m, 2H), 6.31 (s, 1H), |
| | | | 3.98 (s, 2H), 3.60 (dt, J = 18.0 |
| | | | Hz, 5.2 Hz, 4H), 3.42 (t, J = |
| | | | 5.2 Hz, 4H), 3.03 (s, 4H), 2.86 |
| | | | (t, J = 5.2 Hz, 2H), 2.58 (t, J = |
| | | | 5.6 Hz, 2H), 2.45 (t, J = 5.2 |
| | | | Hz, 4H), 1.45 (s, 9H). |
| | | | MS (ESI) m/z 526.7 |
| | | | [C₃₀H₄₀FN₃O₄ + H]⁺. |
| 148 | A | 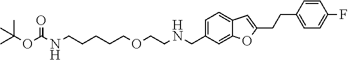 [SMILES: CC(C)(C)OC(=O)NCCCCCOCCNCc1ccc2cc(CCc3ccc(F)cc3)oc2c1] | ¹H NMR (400 MHz, CDCl₃) δ (ppm): 7.44 − 7.37 (m, 2H), 7.19 − 7.10 (m, 3H), 7.01 − 6.90 (m, 2H), 6.30 (s, 1H), |
| | | | 4.55 (s, 1H), 3.93 (s, 2H), |
| | | | 3.56 (t, J = 5.2 Hz, 2H), 3.42 |
| | | | (t, J = 6.4 Hz, 2H), 3.10 (q, J = |
| | | | 6.8 Hz, 2H), 3.03 (s, 4H), |
| | | | 2.83 (t, J = 5.2 Hz, 2H), 1.66 − |
| | | | 1.43 (m, 4H), 1.43 (s, 9H), |
| | | | 1.41 − 1.30 (m, 2H). |
| | | | MS (ESI) m/z 499.6 |
| | | | [C₂₉H₃₉FN₂O₄ + H]⁺. |
| 149 | A | 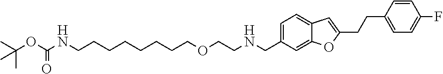 [SMILES: CC(C)(C)OC(=O)NCCCCCCCCOCCNCc1ccc2cc(CCc3ccc(F)cc3)oc2c1] | ¹H NMR (400 MHz, CDCl₃) δ (ppm): 7.44 − 7.36 (m, 2H), 7.19 − 7.10 (m, 3H), 7.01 − 6.90 (m, 2H), 6.30 (s, 1H), 4.51 (s, 1H), 3.92 (s, 2H), |
| | | | 3.56 (t, J = 5.2 Hz, 2H), 3.41 |
| | | | (t, J = 6.8 Hz, 2H), 3.08 (q, J = |
| | | | 6.8 Hz, 2H), 3.03 (s, 4H), |
| | | | 2.83 (t, J = 5.2 Hz, 2H), 1.56 |
| | | | (p, J = 7.2 Hz, 2H), 1.44 (d, J = |
| | | | 12.0 Hz, 2H), 1.44 (s, 9H), |
| | | | 1.35 − 1.23 (m, 8H). |
| | | | MS (ESI) m/z 541.8 |
| | | | [C₃₂H₄₅FN₂O₄ + H]⁺. |
| 150 | A | 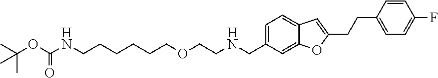 [SMILES: CC(C)(C)OC(=O)NCCCCCCOCCNCc1ccc2cc(CCc3ccc(F)cc3)oc2c1] | ¹H NMR (400 MHz, CDCl₃) δ (ppm): 7.49 (s, 1H), 7.43 (d, J = 8.0 Hz, 1H), 7.21 (d, J = 8.0 Hz, 1H), 7.18 − 7.09 (m, 2H), 7.01 − 6.90 (m, 2H), 6.31 |
| | | | (d, J = 0.8 Hz, 1H), 4.52 (s, |
| | | | 1H), 4.04 (s, 2H), 3.62 (t, J = |
| | | | 5.2 Hz, 2H), 3.43 (t, J = 6.4 |
| | | | Hz, 2H), 3.09 (q, J = 6.8 Hz, |
| | | | 2H), 3.03 (s, 4H), 2.92 (t, J = |
| | | | 5.2 Hz, 2H), 1.56 (p, J = 6.4 |
| | | | Hz, 2H), 1.44 (s, 9H), 1.33 (s, |
| | | | 6H). |
| | | | MS (ESI) m/z 513.7 |
| | | | [C₃₀H₄₁FN₂O₄ + H]⁺. |
| 151 | A | 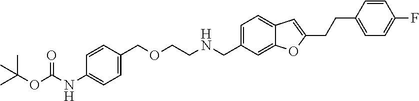 [SMILES: CC(C)(C)OC(=O)Nc1ccc(COCCNCc2ccc3cc(CCc4ccc(F)cc4)oc3c2)cc1] | ¹H NMR (400 MHz, CDCl₃) δ (ppm): 7.43 − 7.36 (m, 2H), 7.33 (d, J = 8.4 Hz, 2H), 7.24 (d, J = 7.2 Hz, 1H), 7.18 − 7.10 (m, 3H), 7.01 − 6.90 (m, 2H), 6.46 (s, 1H), 6.30 (s, 1H), 4.45 (s, 2H), 3.91 (s, |
| | | | 2H), 3.60 (t, J = 5.2 Hz, 2H), |
| | | | 3.03 (s, 4H), 2.86 (t, J = 5.2 |
| | | | Hz, 2H), 1.51 (s, 9H). |
| | | | MS (ESI) m/z 519.6 |
| | | | [C₃₁H₃₅FN₂O₄ + H]⁺. |
| 152 | A | 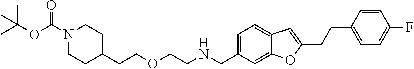 [SMILES: CC(C)(C)OC(=O)N1CCC(CCOCCNCc2ccc3cc(CCc4ccc(F)cc4)oc3c2)CC1] | ¹H NMR (400 MHz, DMSO- d₆) δ (ppm): 7.45 − 7.38 (m, 2H), 7.32 − 7.23 (m, 2H), 7.16 − 7.02 (m, 3H), 6.50 (s, 1H), 3.88 (d, J = 12.8 Hz, 2H), 3.78 |
| | | | (s, 2H), 3.40 (dt, J = 15.2 Hz, |
| | | | 6.0 Hz, 4H), 3.10 − 2.95 (m, |
| | | | 4H), 2.62 (t, J = 5.6 Hz, 4H), |
| | | | 1.63 − 1.55 (m, 2H), 1.55 − |
| | | | 1.37 (m, 3H), 1.37 (s, 9H), |
| | | | 0.95 (qd, J = 12.4 Hz, 4.4 Hz, |
| | | | 2H). |
| | | | MS (ESI) m/z 525.8 |
| | | | [C₃₁H₄₁FN₂O₄ + H]⁺. |
| 153 | A | 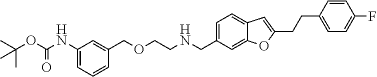 [SMILES: CC(C)(C)OC(=O)Nc1cccc(COCCNCc2ccc3cc(CCc4ccc(F)cc4)oc3c2)c1] | ¹H NMR (400 MHz, CDCl₃) δ (ppm): 7.40 (d, J = 8.0 Hz, 2H), 7.36 (s, 1H), 7.26 (t, J = 3.2 Hz, 2H), 7.19 − 7.09 (m, 3H), 7.04 − 6.90 (m, 3H), 6.53 (s, 1H), 6.30 (d, J = 0.8 Hz, |
| | | | 1H), 4.49 (s, 2H), 3.90 (s, |
| | | | 2H), 3.62 (t, J = 5.2 Hz, 2H), |
| | | | 3.03 (s, 4H), 2.86 (t, J = 5.2 |
| | | | Hz, 2H), 1.51 (s, 9H). |
| | | | MS (ESI) m/z 519.6 |
| | | | [C₃₁H₃₅FN₂O₄ + H]⁺. |
| 154 | A | 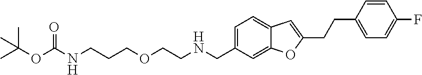 [SMILES: CC(C)(C)OC(=O)NCCCOCCNCc1ccc2cc(CCc3ccc(F)cc3)oc2c1] | ¹H NMR (400 MHz, DMSO- d₆) δ (ppm): 7.45 − 7.38 (m, 2H), 7.33 − 7.25 (m, 2H), 7.17 − 7.03 (m, 3H), 6.77 (d, J = 6.0 Hz, 1H), 6.51 (s, 1H), 3.78 |
| | | | (s, 2H), 3.42 (t, J = 5.6 Hz, |
| | | | 4H), 3.10 − 2.91 (m, 6H), 2.62 |
| | | | (t, J = 5.6 Hz, 2H), 1.58 (p, J = |
| | | | 6.4 Hz, 2H), 1.36 (s, 9H). |
| | | | MS (ESI) m/z 471.7 |
| | | | [C₂₇H₃₅FN₂O₄ + H]⁺. |
| 155 | A | 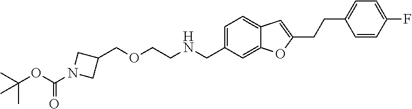 [SMILES: CC(C)(C)OC(=O)N1CC(COCCNCc2ccc3cc(CCc4ccc(F)cc4)oc3c2)C1] | ¹H NMR (400 MHz, DMSO- d₆) δ (ppm): 7.41 (d, J = 8.4 Hz, 2H), 7.32 − 7.24 (m, 2H), 7.17 − 7.04 (m, 3H), 6.50 (s, 1H), 3.84 (s, 2H), 3.78 (s, 2H), 3.53 (s, 2H), 3.51 − 3.44 (m, 4H), 3.03 (dq, J = 13.2 |
| | | | Hz, 6.8 Hz, 4H), 2.75 − 2.67 |
| | | | (m, 1H), 2.64 (t, J = 5.6 Hz, |
| | | | 2H), 1.35 (s, 9H). |
| | | | MS (ESI) m/z 483.7 |
| | | | [C₂₈H₃₅FN₂O₄ + H]⁺. |
| 156 | A | 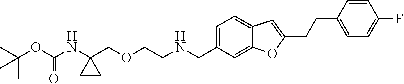 [SMILES: CC(C)(C)OC(=O)NC1(COCCNCc2ccc3cc(CCc4ccc(F)cc4)oc3c2)CC1] | ¹H NMR (400 MHz, CDCl₃) δ (ppm): 7.48 (s, 1H), 7.42 (d, J = 7.8 Hz, 1H), 7.21 (dd, J = 7.9, 1.4 Hz, 1H), 7.18 − 7.10 (m, 2H), 7.01 − 6.91 (m, 2H), 6.31 (s, 1H), 5.40 (s, 1H), |
| | | | 4.03 (s, 2H), 3.68 (t, J = 5.1 |
| | | | Hz, 2H), 3.45 (s, 2H), 3.02 (s, |
| | | | 4H), 2.90 (t, J = 5.1 Hz, 2H), |
| | | | 1.41 (s, 9H), 0.96 − 0.59 (m, |
| | | | 4H). |
| | | | MS (ESI) m/z 483.8 |
| | | | [C₂₈H₃₅FN₂O₄ + H]⁺. |
| 157 | A | 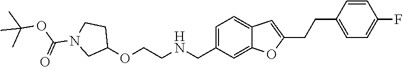 [SMILES: CC(C)(C)OC(=O)N1CCC(OCCNCc2ccc3cc(CCc4ccc(F)cc4)oc3c2)C1] | ¹H NMR (400 MHz, CDCl₃) δ (ppm): 7.43 − 7.37 (m, 2H), 7.18 − 7.10 (m, 3H), 6.95 (t, J = 8.8 Hz, 2H), 6.30 (s, 1H), 4.06 − 3.98 (m, 1H), 3.90 (s, |
| | | | 2H), 3.61 − 3.53 (m, 2H), 3.46 − |
| | | | 3.30 (m, 4H), 3.03 (s, 4H), |
| | | | 2.81 (t, J = 5.2 Hz, 2H), 2.03 − |
| | | | 1.86 (m, 2H), 1.45 (s, 9H). |
| | | | MS (ESI) m/z 483.6 |
| | | | [C₂₈H₃₅FN₂O₄ + H]⁺. |
| 158 | A | 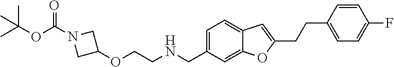 [SMILES: CC(C)(C)OC(=O)N1CC(OCCNCc2ccc3cc(CCc4ccc(F)cc4)oc3c2)C1] | ¹H NMR (400 MHz, CDCl₃) δ (ppm): 7.44 − 7.38 (m, 2H), 7.19 − 7.10 (m, 3H), 7.01 − 6.91 (m, 2H), 6.31 (s, 1H), 4.26 − 4.17 (m, 1H), 4.10 − |
| | | | 4.01 (m, 2H), 3.93 (s, 2H), |
| | | | 3.82 (dd, J = 9.6 Hz, 4.0 Hz, |
| | | | 2H), 3.51 (t, J = 5.2 Hz, 2H), |
| | | | 3.03 (s, 4H), 2.84 (t, J = 5.2 |
| | | | Hz, 2H), 1.43 (s, 9H). |
| | | | MS (ESI) m/z 469.6 |
| | | | [C₂₇H₃₃FN₂O₄ + H]⁺. |
| 159 | A | 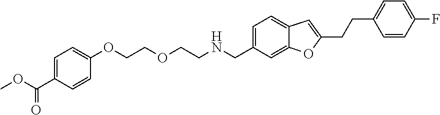 [SMILES: COC(=O)c1ccc(OCCOCCNCc2ccc3cc(CCc4ccc(F)cc4)oc3c2)cc1] | ¹H NMR (400 MHz, CDCl₃) δ (ppm): 8.00 − 7.92 (m, 2H), 7.42 (s, 1H), 7.39 (d, J = 8.0 Hz, 1H), 7.19 − 7.09 (m, 3H), 7.01 − 6.87 (m, 4H), 6.30 (d, J = 0.8 Hz, 1H), 4.20 − 4.13 (m, 2H), 3.94 (s, 2H), 3.87 (s, 3H), 3.86 − 3.80 (m, 2H), 3.72 |
| | | | (t, J = 5.2 Hz, 2H), 3.02 (s, |
| | | | 4H), 2.89 (t, J = 5.2 Hz, 2H). |
| | | | MS (ESI) m/z 492.5 |
| | | | [C₂₉H₃₀FNO₅ + H]⁺. |
| 160 | A | 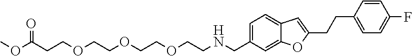 [SMILES: COC(=O)CCOCCOCCOCCNCc1ccc2cc(CCc3ccc(F)cc3)oc2c1] | ¹H NMR (400 MHz, CDCl₃) δ (ppm): 7.44 (s, 1H), 7.40 (d, J = 8.0 Hz, 1H), 7.21 − 7.10 (m, 3H), 7.01 − 6.90 (m, 2H), |
| | | | 6.30 (s, 1H), 3.94 (s, 2H), |
| | | | 3.73 (t, J = 6.4 Hz, 2H), 3.67 |
| | | | (s, 3H), 3.66 − 3.56 (m, 10H), |
| | | | 3.03 (s, 4H), 2.86 (t, J = 5.2 |
| | | | Hz, 2H), 2.58 (t, J = 6.4 Hz, |
| | | | 2H). |
| | | | MS (ESI) m/z 488.8 |
| | | | [C₂₇H₃₄FNO₆ + H]⁺. |
| 161 | A | 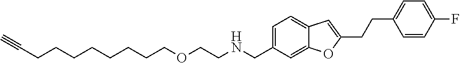 [SMILES: C#CCCCCCCCCOCCNCc1ccc2cc(CCc3ccc(F)cc3)oc2c1] | ¹H NMR (400 MHz, CDCl₃) δ (ppm): 7.45 − 7.38 (m, 2H), 7.20 − 7.09 (m, 3H), 7.01 − 6.91 (m, 2H), 6.31 (d, J = 0.8 |
| | | | Hz, 1H), 3.95 (s, 2H), 3.61 − |
| | | | 3.54 (m, 2H), 3.41 (t, J = 6.8 |
| | | | Hz, 2H), 3.03 (s, 4H), 2.86 (t, |
| | | | J = 5.2 Hz, 2H), 2.17 (td, J = |
| | | | 7.2 Hz, 2.8 Hz, 2H), 2.02 (s, |
| | | | 1H), 1.93 (t, J = 2.8 Hz, 1H), |
| | | | 1.61 − 1.45 (m, 3H), 1.45 − |
| | | | 1.35 (m, 1H), 1.39 − 1.23 (m, |
| | | | 7H). |
| | | | MS (ESI) m/z 450.6 |
| | | | [C₂₉H₃₆FNO₂ + H]⁺. |
| 162 | A | 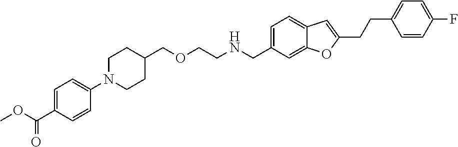 [SMILES: COC(=O)c1ccc(N2CCC(COCCNCc3ccc4cc(CCc5ccc(F)cc5)oc4c3)CC2)cc1] | ¹H NMR (400 MHz, CDCl₃) δ (ppm): 7.92 − 7.84 (m, 2H), 7.48 (s, 1H), 7.42 (d, J = 8.0 Hz, 1H), 7.20 (d, J = 8.0 Hz, 1H), 7.17 − 7.09 (m, 2H), 7.01 − 6.91 (m, 2H), 6.88 − 6.80 (m, 2H), 6.31 (s, 1H), 4.01 (s, 2H), 3.85 (s, 4H), 3.65 − 3.59 (m, 2H), 3.31 (d, J = 6.0 Hz, |
| | | | 2H), 3.02 (s, 4H), 2.92 − 2.88 |
| | | | (m, 2H), 2.83 (t, J = 12.4 Hz, |
| | | | 2H), 1.82 (d, J = 11.2 Hz, 3H), |
| | | | 1.39 − 1.27 (m, 3H). |
| | | | MS (ESI) m/z 545.6 |
| | | | [C₃₃H₃₇FN₂O₄ + H]⁺. |
| 163 | A | 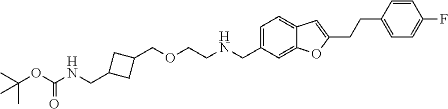 [SMILES: CC(C)(C)OC(=O)NCC1CC(COCCNCc2ccc3cc(CCc4ccc(F)cc4)oc3c2)C1] | ¹H NMR (400 MHz, CDCl₃) δ (ppm): 7.40 (d, J = 8.0 Hz, 2H), 7.19 − 7.09 (m, 3H), 7.01 − 6.90 (m, 2H), 6.30 (d, J = 0.8 Hz, 1H), 4.53 (s, 1H), 3.91 (s, 2H), 3.55 (t, J = 5.2 Hz, 2H), 3.35 (d, J = 6.4 Hz, 2H), |
| | | | 3.08 (t, J = 6.4 Hz, 2H), 3.03 |
| | | | (s, 4H), 2.81 (t, J = 5.2 Hz, |
| | | | 2H), 2.49 − 2.26 (m, 2H), |
| | | | 2.18 − 2.06 (m, 2H), 1.50 − 1.44 |
| | | | (m, 2H), 1.43 (s, 9H). |
| | | | MS (ESI) m/z 511.6 |
| | | | [C₃₀H₃₉FN₂O₄ + H]⁺. |
| 164 | A | 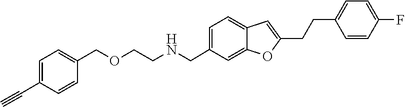 [SMILES: C#Cc1ccc(COCCNCc2ccc3cc(CCc4ccc(F)cc4)oc3c2)cc1] | ¹H NMR (400 MHz, DMSO- d₆) δ (ppm): 7.48 − 7.39 (m, 4H), 7.37 − 7.24 (m, 4H), 7.17 − 7.03 (m, 3H), 6.51 (d, J = 0.8 Hz, 1H), 4.48 (s, 2H), 4.16 (s, 1H), 3.79 (s, 2H), 3.51 (t, J = 5.6 Hz, 2H), 3.11 − 2.95 (m, |
| | | | 4H), 2.69 (t, J = 5.6 Hz, 2H). |
| | | | MS (ESI) m/z 428.5 |
| | | | [C₂₈H₂₆FNO₂ + H]⁺. |
| 165 | A | 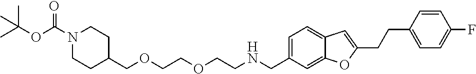 [SMILES: CC(C)(C)OC(=O)N1CCC(COCCOCCNCc2ccc3cc(CCc4ccc(F)cc4)oc3c2)CC1] | ¹H NMR (400 MHz, DMSO- d₆) δ (ppm): 7.47 − 7.40 (m, 2H), 7.33 − 7.24 (m, 2H), 7.19 − 7.01 (m, 3H), 6.52 (s, 1H), 3.89 (d, J = 12.8 Hz, 2H), 3.82 |
| | | | (s, 2H), 3.53 − 3.43 (m, 6H), |
| | | | 3.22 (d, J = 6.4 Hz, 2H), 3.11 − |
| | | | 2.96 (m, 4H), 2.68 (t, J = 5.6 |
| | | | Hz, 2H), 2.66 − 2.52 (m, 2H), |
| | | | 1.69 − 1.55 (m, 3H), 1.37 (s, |
| | | | 9H), 0.97 (qd, J = 12.4 Hz, 4.4 |
| | | | Hz, 2H). |
| | | | MS (ESI) m/z 555.9 |
| | | | [C₃₂H₄₃FN₂O₅ + H]⁺. |
| 166 | A | 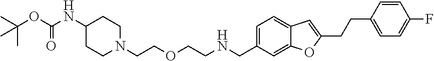 [SMILES: CC(C)(C)OC(=O)NC1CCN(CCOCCNCc2ccc3cc(CCc4ccc(F)cc4)oc3c2)CC1] | ¹H NMR (400 MHz, CDCl₃) δ (ppm): 7.39 (d, J = 8.0 Hz, 2H), 7.18 − 7.09 (m, 3H), 7.00 − 6.90 (m, 2H), 6.30 (d, J = |
| | | | 0.8 Hz, 1H), 4.41 (s, 1H), 3.90 |
| | | | (s, 2H), 3.61 − 3.51 (m, 4H), |
| | | | 3.44 (s, 1H), 3.03 (s, 4H), |
| | | | 2.89 − 2.78 (m, 4H), 2.55 (t, J = |
| | | | 5.6 Hz, 2H), 2.11 (t, J = 11.6 |
| | | | Hz, 2H), 1.93 − 1.84 (m, 2H), |
| | | | 1.43 (s, 9H), 1.47 − 1.33 (m, |
| | | | 2H). |
| | | | MS (ESI) m/z 540.7 |
| | | | [C₃₁H₄₂FN₃O₄ + H]⁺. |
| 167 | A | 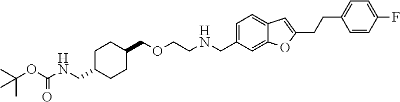 [SMILES: CC(C)(C)OC(=O)NC[C@H]1CC[C@H](COCCNCc2ccc3cc(CCc4ccc(F)cc4)oc3c2)CC1] | ¹H NMR (400 MHz, CDCl₃) δ (ppm): 7.40 (d, J = 8.0 Hz, 2H), 7.18 − 7.10 (m, 3H), 7.01 − 6.90 (m, 2H), 6.30 (s, 1H), 4.58 (s, 1H), 3.90 (s, 2H), 3.54 (t, J = 5.2 Hz, 2H), 3.23 (d, J = 6.4 Hz, 2H), 3.03 (s, |
| | | | 4H), 2.96 (t, J = 6.4 Hz, 2H), |
| | | | 2.81 (t, J = 5.2 Hz, 2H), 1.82 − |
| | | | 1.74 (m, 4H), 1.44 (s, 9H), |
| | | | 1.37 (s, 1H), 1.00 − 0.84 (m, 5H). |
| | | | MS (ESI) m/z 539.7 |
| | | | [C₃₂H₄₃FN₂O₄ + H]⁺. |
| 168 | A | 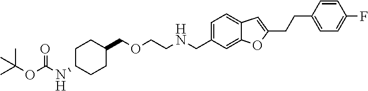 [SMILES: CC(C)(C)OC(=O)N[C@H]1CC[C@H](COCCNCc2ccc3cc(CCc4ccc(F)cc4)oc3c2)CC1] | ¹H NMR (400 MHz, CDCl₃) δ (ppm): 7.43 − 7.37 (m, 2H), 7.18 − 7.10 (m, 3H), 7.01 − 6.90 (m, 2H), 6.30 (s, 1H), 4.64 (s, 1H), 3.90 (s, 2H), 3.73 (s, 1H), 3.54 (t, J = 5.2 |
| | | | Hz, 2H), 3.28 (d, J = 6.4 Hz, |
| | | | 2H), 3.03 (s, 4H), 2.81 (t, J = |
| | | | 5.2 Hz, 2H), 1.69 − 1.55 (m, |
| | | | 8H), 1.44 (s, 9H), 1.29 − 1.16 |
| | | | (m, 2H). |
| | | | MS (ESI) m/z 525.7 |
| | | | [C₃₁H₄₁FN₂O₄ + H]⁺. |

TABLE 6
Summary of compounds (series III) prepared according to the examples above and characterizations thereof

| | Syn- | |
|---|---|---|
| Cpd | thesis | |
| No. | Route | |
| 100 | M | 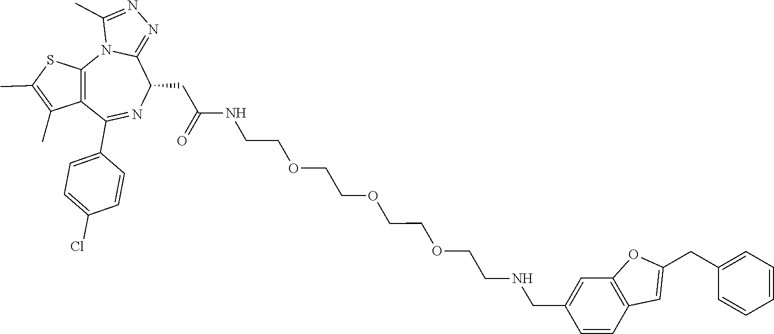 [SMILES: Cc1sc2c(c1C)C(c1ccc(Cl)cc1)=N[C@@H](CC(=O)NCCOCCOCCOCCNCc1ccc3cc(Cc4ccccc4)oc3c1)c1nnc(C)n1-2] |
| 101 | M | 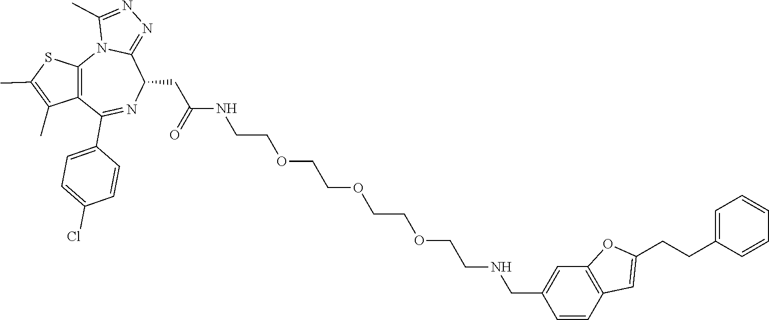 [SMILES: Cc1sc2c(c1C)C(c1ccc(Cl)cc1)=N[C@@H](CC(=O)NCCOCCOCCOCCNCc1ccc3cc(CCc4ccccc4)oc3c1)c1nnc(C)n1-2] |
| 102 | M | 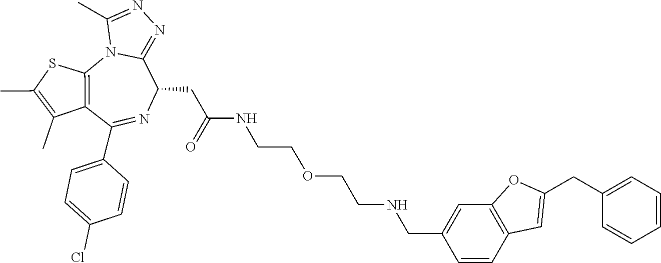 [SMILES: Cc1sc2c(c1C)C(c1ccc(Cl)cc1)=N[C@@H](CC(=O)NCCOCCNCc1ccc3cc(Cc4ccccc4)oc3c1)c1nnc(C)n1-2] |
| 103 | M | 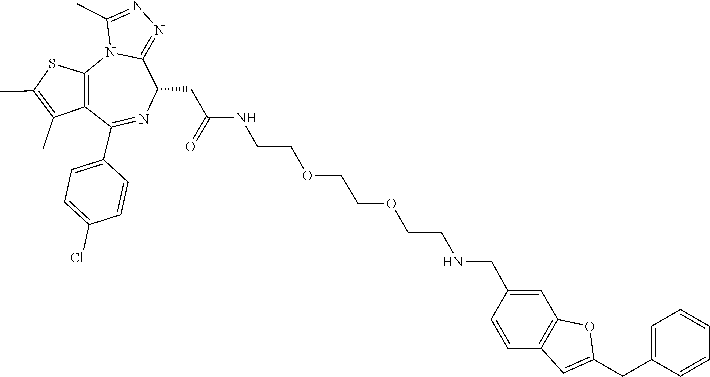 [SMILES: Cc1sc2c(c1C)C(c1ccc(Cl)cc1)=N[C@@H](CC(=O)NCCOCCOCCNCc1ccc3cc(Cc4ccccc4)oc3c1)c1nnc(C)n1-2] |
| 104 | M | 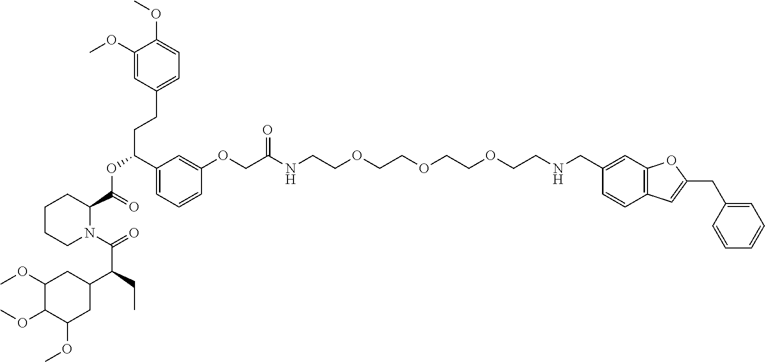 [SMILES: CC[C@H](C(=O)N1CCCC[C@H]1C(=O)O[C@H](CCc1ccc(OC)c(OC)c1)c1cccc(OCC(=O)NCCOCCOCCOCCNCc2ccc3cc(Cc4ccccc4)oc3c2)c1)C1CC(OC)C(OC)C(OC)C1] |
| 169 | M | 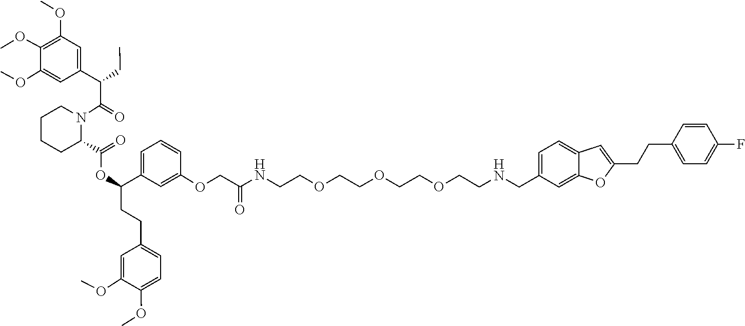 [SMILES: CC[C@H](C(=O)N1CCCC[C@H]1C(=O)O[C@H](CCc1ccc(OC)c(OC)c1)c1cccc(OCC(=O)NCCOCCOCCOCCNCc2ccc3cc(CCc4ccc(F)cc4)oc3c2)c1)c1cc(OC)c(OC)c(OC)c1] |
| 170 | M | 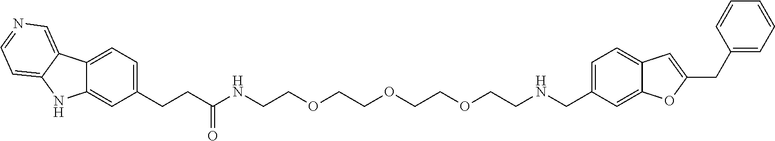 [SMILES: O=C(CCc1ccc2c(c1)[nH]c1ccncc12)NCCOCCOCCOCCNCc1ccc2cc(Cc3ccccc3)oc2c1] |
| 171 | M | 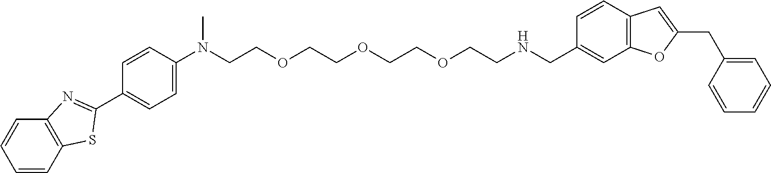 [SMILES: CN(CCOCCOCCOCCNCc1ccc2cc(Cc3ccccc3)oc2c1)c1ccc(-c2nc3ccccc3s2)cc1] |
| 172 | M | 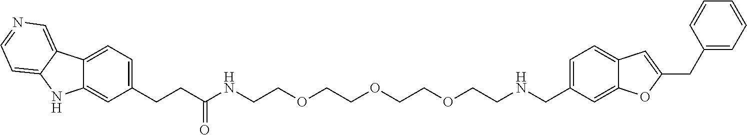 [SMILES: O=C(CCc1ccc2c(c1)[nH]c1ccncc12)NCCOCCOCCOCCNCc1ccc2cc(Cc3ccccc3)oc2c1] |
| 173 | M | 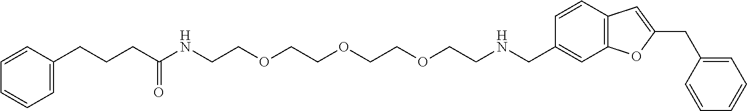 [SMILES: O=C(CCCc1ccccc1)NCCOCCOCCOCCNCc1ccc2cc(Cc3ccccc3)oc2c1] |
| 174 | M | 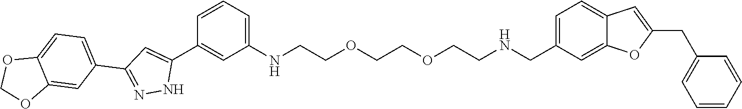 [SMILES: c1ccc(Cc2cc3ccc(CNCCOCCOCCNc4cccc(-c5cc(-c6ccc7c(c6)OCO7)n[nH]5)c4)cc3o2)cc1] |
| 175 | M | 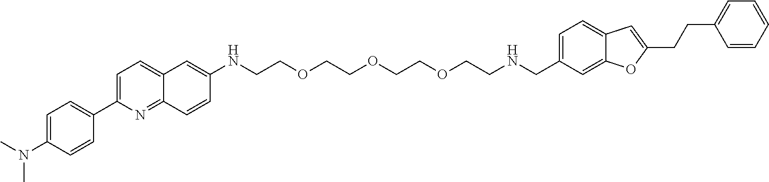 [SMILES: CN(C)c1ccc(-c2ccc3cc(NCCOCCOCCOCCNCc4ccc5cc(CCc6ccccc6)oc5c4)ccc3n2)cc1] |
| 176 | M | 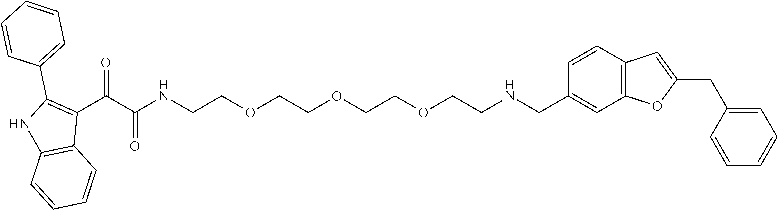 [SMILES: O=C(NCCOCCOCCOCCNCc1ccc2cc(Cc3ccccc3)oc2c1)C(=O)c1c(-c2ccccc2)[nH]c2ccccc12] |
| 177 | M | 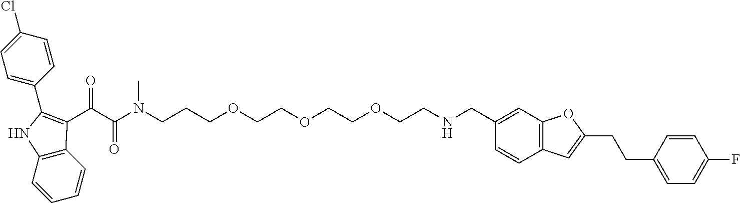 [SMILES: CN(CCCOCCOCCOCCNCc1ccc2cc(CCc3ccc(F)cc3)oc2c1)C(=O)C(=O)c1c(-c2ccc(Cl)cc2)[nH]c2ccccc12] |
| 178 | M | 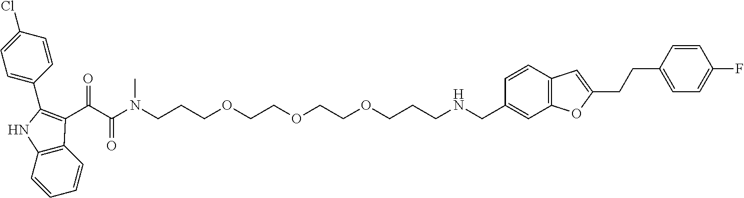 [SMILES: CN(CCCOCCOCCOCCCNCc1ccc2cc(CCc3ccc(F)cc3)oc2c1)C(=O)C(=O)c1c(-c2ccc(Cl)cc2)[nH]c2ccccc12] |
| 179 | M | 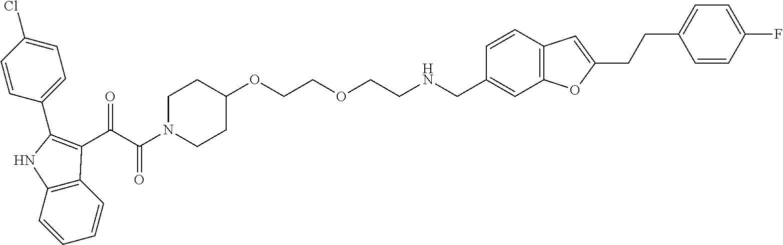 [SMILES: O=C(C(=O)N1CCC(OCCOCCNCc2ccc3cc(CCc4ccc(F)cc4)oc3c2)CC1)c1c(-c2ccc(Cl)cc2)[nH]c2ccccc12] |
| 180 | M | 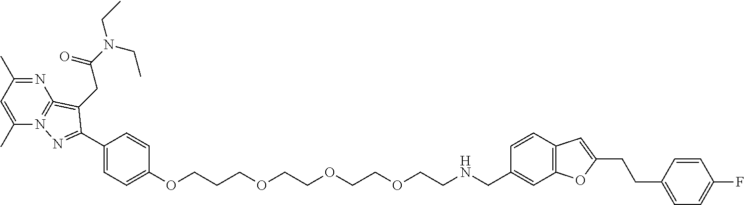 |
| 181 | M | 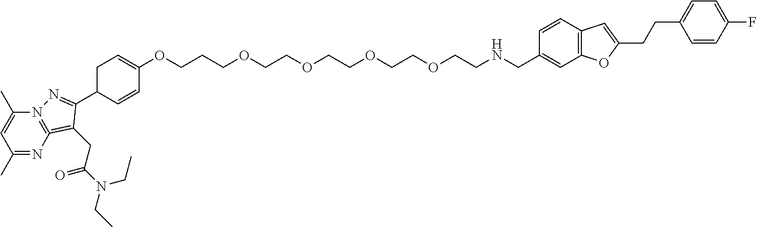 |
| 182 | M | 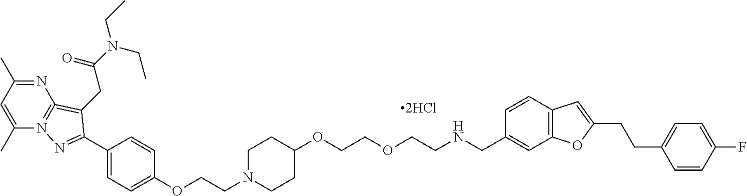 |

| Cpd No. | NMR and Mass Spectrometry |
|---|---|
| 100 | ¹H NMR (400 MHz, DMSO-d₆) δ (ppm): 8.27 (t, J = 5.6 Hz, 1H), 7.49-7.45 |
| | (m, 4H), 7.43-7.40 (m, 2H), 7.35-7.29 (m, 4H), 7.26-7.22 (m, 1H), 7.17 (dd, J = |
| | 8.0 Hz, 1.2 Hz, 1H), 6.57 (s, 1H), 4.52-4.49 (m, 1H), 4.13 (s, 2H), 3.86 (s, 2H), |
| | 3.52-3.42 (m, 12H), 3.30-3.19 (m, 4H), 2.70 (t, J = 5.6 Hz, 2H), 2.59 (s, 3H), |
| | 2.40 (s, 3H), 1.61 (s, 3H). |
| | MS (ESI) m/z 795.4 [C₄₃H₄₇ClN₆O₅S + H]⁺. |
| 101 | ¹H NMR (400 MHz, DMSO-d₆) δ (ppm): 8.27 (t, J = 5.6 Hz, 1H), 7.51-7.41 |
| | (m, 6H), 7.29-7.24 (m, 4H), 7.20-7.16 (m, 2H), 6.54 (s, 1H), 4.52-4.49 (m, 1H), |
| | 3.95 (s, 2H), 3.56-3.52 (m, 10H), 3.46-3.43 (m, 2H), 3.24-3.19 (m, 4H), 3.10- |
| | 3.06 (m, 2H), 3.03-2.99 (m, 2H), 2.79 (t, J = 5.6 Hz, 2H), 2.59 (s, 3H), 2.40 (s, |
| | 3H), 1.61 (s, 3H). |
| | MS (ESI) m/z 809.3 [C₄₄H₄₉ClN₆O₅S + H]⁺. |
| 102 | ¹H NMR (400 MHz, DMSO-d₆) δ (ppm): 8.29 (t, J = 5.6 Hz, 1 H), 8.21 (s, |
| | 1H; HCOOH), 7.49-7.47 (m, 4H), 7.42-7.40 (m, 2H), 7.35-7.28 (m, 4H), 7.26- |
| | 7.19 (m, 2H), 6.57 (s, 1H), 4.52-4.49 (m, 1H), 4.13 (s, 2H), 3.91 (s, 2H), 3.54 (t, J = |
| | 5.6 Hz, 2H), 3.45 (t, J = 5.6 Hz, 2H), 3.34-3.19 (m, 4H), 2.76 (t, J = 5.6 Hz, |
| | 2H), 2.59 (s, 3H), 2.40 (s, 3H), 1.61 (s, 3H). |
| | MS (ESI) m/z 707.2 [C₃₉H₃₉ClN₆O₃S + H]⁺. |
| 103 | ¹H NMR (400 MHz, DMSO-d₆) δ (ppm): 8.27 (t, J = 5.6 Hz, 1H), 8.18 (s, 1H; |
| | HCOOH), 7.49-7.46 (m, 4H), 7.42-7.40 (m, 2H), 7.35-7.29 (m, 4H), 7.26- |
| | 7.22 (m, 1H), 7.18 (dd, J = 8.0 Hz, 1.2 Hz, 1H), 6.57 (s, 1H), 4.52-4.48 (m, 1H), |
| | 4.13 (s, 2H), 3.89 (s, 2H), 3.53-3.47 (m, 6H), 3.45 (t, J = 6.0 Hz, 2H), 3.30-3.22 |
| | (m, 4H), 2.74 (t, J = 5.6 Hz, 2H), 2.59 (s, 3H), 2.40 (s, 3H), 1.61 (s, 3H). |
| | MS (ESI) m/z 751.3 [C₄₁H₄₃ClN₆O₄S + H]⁺. |
| 104 | ¹H NMR (400 MHz, DMSO-d₆) δ (ppm): 8.05 (t, J = 5.6 Hz, 1H), 7.50- |
| | 7.48 (m, 2H), 7.34-7.15 (m, 7H), 6.84-6.81 (m, 3H), 6.75-6.72 (m, 1H), 6.64- |
| | 6.58 (m, 3H), 6.52 (s, 2H), 5.54-5.51 (m, 1H), 5.28-5.26 (m, 1H), 4.48-4.46 (m, |
| | 2H), 4.13 (s, 2H), 4.02-3.82 (m, 4H), 3.74-3.70 (m, 8H), 3.58-3.56 (m, 8H), 3.49- |
| | 3.47 (m, 10H), 3.41 (t, J = 6.0 Hz, 2H), 3.28-3.26 (m, 2H), 2.75 (br. s, 2H), 2.64- |
| | 2.58 (m, 1H), 2.38-2.34 (m, 1H), 2.19-2.15 (m, 1H), 1.95-1.87 (m, 3H), 1.64- |
| | 1.54 (m, 4H), 1.39-1.36 (m, 1H), 0.80 (t, J = 7.2 Hz, 3H). |
| | MS (ESI) m/z 1088.8 [C₆₂H₇₇N₃O₁₄ + H]⁺. |
| 169 | ¹H NMR (400 MHz, DMSO-d₆) δ (ppm): 8.07 (t, J = 5.2 Hz, 1H), 7.57 (s, |
| | 1H), 7.50 (d, J = 8.0 Hz, 1H), 7.34-7.22 (m, 3H), 7.19-7.15 (m, 1H), 7.08 (t, |
| | J = 8.8 Hz, 2H), 6.98-6.89 (m, 1H), 6.85-6.81 (m, 2H), 6.75-6.66 (m, 2H), |
| | 6.64-6.52 (m, 4H), 5.56-5.51 (m, 1H), 5.27 (t, J = 4.0 Hz, 1H), 4.49-4.42 (m, |
| | 2H), 4.05-3.98 (m, 3H), 3.86-3.82 (m, 1H), 3.74-3.72 (m, 2H), 3.71 (s, 6H), |
| | 3.63 (s, 1H), 3.59-3.52 (m, 8H), 3.51-3.50 (m, 4H), 3.48-3.44 (m, 4H), 3.42- |
| | 3.40 (m, 2H), 3.28-3.27 (m, 2H), 3.10-3.05 (m, 2H), 3.02-2.98 (m, 2H), |
| | 2.88 (t, J = 4.8 Hz, 2H), 2.68-2.60 (m, 1H), 2.48-2.46 (m, 1H), 2.38-2.31 |
| | (m, 1H), 2.20-2.14 (m, 1H), 2.08-1.82 (m, 3H), 1.68-1.46 (m, 4H), 1.40- |
| | 1.32 (m, 1H), 1.22-0.90 (m, 2H), 0.80 (t, J = 7.2 Hz, 3H). |
| | MS (ESI) m/z 1120.7 [C₆₃H₇₈FN₃O₁₄ + H]⁺. |
| 170 | ¹H NMR (400 MHz, DMSO-d₆) δ (ppm): 11.6 (br. s, 1H), 9.26 (s, 1H), |
| | 8.37 (d, J = 5.2 Hz, 1H), 8.15 (s, 1H; HCOOH), 8.08 (d, J = 8.0 Hz, 1H), 7.89 (t, |
| | J = 5.2 Hz, 1H), 7.53-7.51 (m, 2H), 7.42 (d, J = 5.2 Hz, 1H), 7.35-7.28 (m, 5H), |
| | 7.26-7.21 (m, 2H), 7.10 (dd, J = 8.0 Hz, 1.2 Hz, 1H), 6.59 (s, 1H), 4.14 (s, 2H), |
| | 3.99 (s, 2H), 3.53 (t, J = 5.2 Hz, 2H), 3.51-3.45 (m, 4H), 3.42-3.38 (m, 4H), |
| | 3.33 (t, J = 5.6 Hz, 2H), 3.19-3.14 (m, 2H), 2.97 (t, J = 8.0 Hz, 2H), 2.82 (t, J = |
| | 5.2 Hz, 2H), 2.45 (t, J = 7.6 Hz, 2H). |
| | MS (ESI) m/z 635.3 [C₃₈H₄₂N₄O₅ + H]⁺. |
| 171 | ¹H NMR (400 MHz, DMSO-d₆) δ (ppm): 8.02 (d, J = 8.0 Hz, 1H), 7.91 (d, |
| | J = 8.0 Hz, 1H), 7.85 (d, J = 8.8 Hz, 2H), 7.48-7.41 (m, 3H), 7.37-7.29 (m, 5H), |
| | 7.25-7.21 (m, 1H), 7.14 (d, J = 8.0 Hz, 1H), 6.80 (d, J = 5.2 Hz, 2H), 6.54 (s, 1H), |
| | 4.11 (s, 2H), 3.78 (s, 2H), 3.57 (br. s, 4H), 3.49-3.44 (m, 11H), 2.99 (s, 3H), 2.62 |
| | (t, J = 5.6 Hz, 2H). |
| | MS (ESI) m/z 636.4 [C₃₈H₄₁N₃O₄S + H]⁺. |
| 172 | ¹H NMR (400 MHz, DMSO-d₆) δ (ppm): 11.58 (s, 1H), 9.26 (s, 1H), 8.37 |
| | (d, J = 5.2 Hz, 1H), 8.08 (d, J = 7.6 Hz, 1H), 7.88 (t, J = 5.2 Hz, 1H), 7.42-7.40 (m, |
| | 3H), 7.35 (s, 1H), 7.29-7.24 (m, 4H), 7.19-7.15 (m, 1H), 7.11 (t, J = 7.6 Hz, 2H), |
| | 6.50 (s, 1H), 3.77 (s, 2H), 3.46-3.42 (m, 10H), 3.36-3.34 (m, 2H), 3.20-3.16 (m, |
| | 2H), 3.08-2.95 (m, 6H), 2.62 (t, J = 5.6 Hz, 2H), 2.45 (t, J = 7.6 Hz, 2H). |
| | MS (ESI) m/z 649.4 [C₃₉H₄₄N₄O₅ + H]⁺. |
| 173 | ¹H NMR (400 MHz, DMSO-d₆) δ (ppm): 8.26 (s, 1H; HCOOH), 7.83 (d, J = |
| | 5.2 Hz, 1H), 7.45 (d, J = 7.6 Hz, 1H), 7.41 (s, 1H), 7.35-7.23 (m, 7H), 7.17-7.13 (m, |
| | 4H), 6.55 (s, 1H), 4.12 (s, 2H), 3.77 (s, 2H), 3.48-3.43 (m, 10H), 3.39-3.36 (m, |
| | 2H), 3.19-3.14 (m, 2H), 2.61 (t, J = 5.6 Hz, 2H), 2.07 (t, J = 5.6 Hz, 2H), |
| | 1.80-1.74 (s, 2H). |
| | MS (ESI) m/z 559.4 [C₃₄H₄₂N₂O₅ + H]⁺. |
| 174 | ¹H NMR (400 MHz, DMSO-d₆) δ (ppm): 8.19 (s, 1H; HCOOH), 7.44 (d, J = |
| | 8.0 Hz, 1H), 7.42 (s, 1H), 7.37 (d, J = 1.6 Hz, 1H), 7.34-7.28 (m, 5H), 7.25- |
| | 7.22 (m, 1H), 7.14 (d, J = 8.0 Hz, 1H), 7.09 (d, J = 8.0 Hz, 1H), 6.99-6.95 (m, |
| | 4H), 6.57-6.54 (m, 2H), 6.05 (s, 2H), 6.00 (br. s, 1H), 4.11 (s, 2H), 3.79 (s, |
| | 2H), 3.60-3.47 (m, 10H), 3.27-3.23 (m, 2H), 2.65 (t, J = 5.6 Hz, 2H). |
| | MS (ESI) m/z 631.4 [C₃₈H₃₈N₄O₅ + H]⁺. |
| 175 | ¹H NMR (400 MHz, DMSO-d₆) δ (ppm): 8.21 (s, 1H; HCOOH), 8.02 (d, J = |
| | 8.8 Hz, 2H), 7.95 (d, J = 8.8 Hz, 1H), 7.80 (d, J = 8.8 Hz, 1H), 7.67 (d, J = 9.2 Hz, |
| | 1H), 7.43 (s, 1H), 7.41 (d, J = 8.0 Hz, 1H), 7.28-7.23 (m, 4H), 7.20-7.17 (m, 2H), |
| | 7.13 (d, J = 8.8 Hz, 1H), 6.80 (d, J = 8.8 Hz, 2H), 6.69 (d, J = 2.4 Hz, 1H), |
| | 6.49 (s, 1H), 6.04 (t, 1H), 3.79 (s, 2H), 3.63 (t, J = 5.6 Hz, 2H), 3.56-3.46 (m, |
| | 12H), 3.07-3.04 (m, 2H), 3.01-2.98 (m, 2H), 2.97 (s, 6H), 2.65 (t, J = 5.6 Hz, 2H). |
| | MS (ESI) m/z 673.5 [C₄₂H₄₈N₄O₄ + H]⁺. |
| 176 | ¹H NMR (400 MHz, DMSO-d₆) δ (ppm): 12.35 (s, 1H), 8.50 (t, J = 5.6 Hz, |
| | 1H), 8.08 (dd, J = 7.2 Hz, 1.2 Hz, 1H), 7.58-7.54 (m, 4H), 7.49-7.45 (m, 4H), |
| | 7.35-7.21 (m, 8H), 6.63 (s, 1H), 4.15 (s, 2H), 4.10 (br. s, 2H), 3.59 (t, J = 5.2 Hz, |
| | 2H), 3.53 (s, 4H), 3.51-3.49 (m, 2H), 3.46-3.44 (m, 2H), 3.22 (t, J = 6.0 Hz, 2H), |
| | 2.92-2.87 (m, 4H). |
| | MS (ESI) m/z 660.5 [C₄₀H₄₁N₃O₆ + H]⁺. |
| 177 | ¹H NMR (400 MHz, DMSO-d₆) δ (ppm): 12.55 (br. s, 1H), 8.11-8.03 (m, |
| | 1H), 7.59-7.55 (m, 4H), 7.49 (d, J = 7.2 Hz, 1H), 7.43-7.40 (m, 2H), 7.32- |
| | 7.23 (m, 4H), 7.14-7.06 (m, 3H), 6.50 (s, 1H), 3.77 (d, J = 8.0 Hz, 2H), 3.51-3.37 |
| | (m, 8H), 3.30-3.25 (m, 2H), 3.13 (t, 1H), 3.07-2.95 (m, 5H), 2.78 & 2.54 (2 s, 3H), |
| | 2.65-2.59 (m, 2H), 2.45 (m, 2H), 1.69-1.63 (m, 1H), 1.48-1.42 (m, 1H). |
| | MS (ESI) m/z 754.4 [C₄₃H₄₅ClFN₃O₆ + H]⁺. |
| 178 | ¹H NMR (400 MHz, DMSO-d₆) δ (ppm): 12.55 (br. s, 1H), 8.11-8.03 (m, |
| | 1H), 7.60-7.55 (m, 4H), 7.49 (d, J = 8.0 Hz, 1H), 7.42-7.40 (m, 2H), 7.32- |
| | 7.24 (m, 4H), 7.13 (d, J = 7.6 Hz, 1H), 7.08 (t, J = 8.8 Hz, 2H), 6.50 (s, 1H), 3.77 |
| | (s, 2H), 3.49-3.25 (m, 16H), 3.14 (t, J = 7.2 Hz, 1H), 3.07-2.96 (m, 5H), |
| | 2.79 & 2.45 (2 s, 3H), 2.65-2.61 (m, 2H), 1.68-1.63 (m, 1H), 1.47-1.43 (m, 1H). |
| | MS (ESI) m/z 798.6 [C₄₅H₄₉ClFN₃O₇+ H]⁺. |
| 179 | ¹H NMR (400 MHz, DMSO-d₆) δ (ppm): 12.55 (br. s, 1H), 8.08 (d, J = 7.6 |
| | Hz, 1H), 7.61-7.54 (m, 4H), 7.50 (d, J = 7.2 Hz, 1H), 7.42 (s, 1H), 7.39 (d, J = 8.4 |
| | Hz, 1H), 7.32-7.25 (m, 4H), 7.13-7.06 (m, 3H), 6.46 (s, 1H), 3.77 (s, 2H), 3.48-3.44 |
| | (m, 7H), 3.31-3.26 (m, 2H), 3.03-2.97 (m, 5H), 2.89-2.83 (m, 1H), 2.63 (t, J = |
| | 5.6 Hz, 2H), 2.13 (br, 1H), 1.64-1.62 (m, 2H), 1.27-1.21 (m, 2H). |
| | MS (ESI) m/z 722.4 [C₄₂H₄₁ClFN₃O₅ + H]⁺. |
| 180 | ¹H NMR (400 MHz, DMSO-d₆) δ (ppm): 7.68 (d, J = 8.8 Hz, 2H), 7.41- |
| | 7.39 (m, 2H), 7.29-7.25 (m, 2H), 7.13-7.05 (m, 3H), 7.00 (d, J = 8.8 Hz, 2H), |
| | 6.84 (s, 1H), 6.49 (s, 1H), 4.05 (t, J = 6.4 Hz, 2H), 3.82 (s, 2H), 3.76 (s, 2H), |
| | 3.56-3.44 (m, 14H), 3.31-3.25 (m, 2H), 3.04-2.99 (m, 4H), 2.68 (s, 3H), 2.62 (t, J = |
| | 5.6 Hz, 2H), 2.46 (s, 3H), 1.95-1.92 (m, 2H), 1.17 (t, J = 7.2 Hz, 3H), 1.01 (t, J = |
| | 7.2 Hz, 3H). |
| | MS (ESI) m/z 794.3 [C₄₆H₅₆FN₅O₆ + H]⁺. |
| 181 | ¹H NMR (400 MHz, DMSO-d₆) δ (ppm): 7.68 (d, J = 8.8 Hz, 2H), 7.41- |
| | 7.39 (m, 2H), 7.29-7.25 (m, 2H), 7.13-7.07 (m, 3H), 7.01 (d, J = 8.8 Hz, 2H), |
| | 6.83 (s, 1H), 6.49 (s, 1H), 4.05 (t, 2H), 3.83 (s, 2H), 3.75 (s, 2H), 3.55-3.44 (m, |
| | 20H), 3.29-3.27 (m, 4H), 3.04-3.00 (m, 4H), 2.69 (s, 3H), 2.61 (t, J = 5.6 Hz, |
| | 2H), 1.98-1.93 (m, 2H), 1.17 (t, J = 7.2 Hz, 3H), 1.01 (t, J = 7.2 Hz, 3H). |
| | MS (ESI) m/z 838.6 [C₄₈H₆₀FN₅O₇ + H]⁺. |
| 182 | ¹H NMR (400 MHz, DMSO-d₆) δ (ppm): 10.69-10.45 (m, 1H), 9.32-9.15 |
| | (m, 2H), 7.76-7.72 (m, 3H), 7.56 (dd, J = 8.0 Hz, 3.6 Hz, 1H), 7.35 (d, J = 8.0 Hz, |
| | 1H), 7.30-7.26 (m, 2H), 7.11-7.06 (m, 4H), 6.86 (s, 1H), 6.60 (s, 1H), 4.46-4.42 |
| | (m, 2H), 4.29 (br. s, 2H), 3.85 (s, 2H), 3.75-3.70 (m, 3H), 3.59-3.50 (m, 10H), |
| | 3.40-3.37 (m, 1H), 3.28 (q, J = 6.8 Hz, 2H), 3.18-3.01 (m, 7H), 2.70 (s, 3H), 2.49 |
| | (s, 3H), 2.15-2.10 (1H), 1.97 (br, 2H), 1.79-1.69 (m, 1H), 1.19 (t, J = 6.8 Hz, |
| | 3H), 1.02 (t, J = 6.8 Hz, 3H). |
| | MS (ESI) m/z 819.5 [C₄₈H₅₉FN₆O₅ + H]⁺. |

### Example 28: Surface Plasmon Resonance (SPR) Analysis

### Protein Preparation

Recombinant p62 protein was buffer exchanged into Biacore SPR running buffer (Cytiva 100671) using the Slide-A-Lyzer Mini Dialysis system 7K MWCO (Thermo Fisher Scientific, Cat no. 69560). The final protein concentration was determined using the Nanodrop One spectrophotometer (Thermo Fisher Scientific, cat no. ND-ONE-W), with calibration for the dialysis buffer.

### Protein Immobilisation

The prepared protein was diluted in sodium acetate buffer (NaAc, pH 5.5, Cytiva) to concentrations ranging from approximately 0.25-40 μg/mL. Post-immobilization, the protein required approximately 16 hours of equilibration in running buffer supplemented with 1% DMSO at 15° C. to ensure adequate surface equilibration and protein reorganization.

### SPR Running Conditions

All SPR experiments were conducted at a constant temperature of 15° C. The SPR chip surface was activated using EDC/NHS for a contact time of 6-10 min at a flow rate of 10 μL/min. This was followed by protein immobilization for 2100 seconds and ethanolamine capping for 420 seconds, both at the same flow rate. Immediately after immobilization, the NH₂—REEE peptide was analyzed in Single Cycle Kinetics mode (SCK) to verify surface activity, ensuring it binds the sample with the published affinity (−20 μM by SPR).

### Compound Binding Experiments

For general compound binding studies, experiments were performed at 15° C. using a running buffer composed of 10 mM HEPES, 150 mM NaCl, 1 mM DTT, 0.05% Tween 20, and 1% DMSO at pH 7.4. The test compound was initially prepared at a 20 mM concentration in DMSO and subsequently titrated in 2-fold dilutions. For each assay, 2 μL of the compound was mixed with 198 μL of the running buffer, and the mixture was thoroughly pipetted approximately 15 times for homogenization.

A series of blank injections (0 μM concentration) were included for double referencing, with at least 8 or 16 per plate depending on the format. Typical injection parameters were 120 seconds for association and 180 seconds for dissociation at a flow rate of 50 L/min. A four-point solvent correction was prepared with DMSO concentrations ranging from 2% to 0.5% in the buffer. Post each compound injection, the system's needles were washed with 50% DMSO.

### Data Acquisition and Analysis

Analysis of the SPR data was conducted using the Cytiva Evaluation software. The binding levels at the late time-point of each interaction were corrected for reference, then normalized to percentage bound values to account for variations in immobilization levels, protein molecular weight, and compound molecular weight.

### Example 29: Western Blot Protocol

HeLa cells were seeded in 6-well plates (700,000 cells) and allowed to adhere overnight. Media was replaced one hour before administering either vehicle (DMSO) or test compounds. After 24 hours of treatment, the media was discarded, and cells were rinsed once with ice-cold 1×PBS before detached from plates with ice-cold RIPA Lysis and Extraction Buffer (Thermo Fisher Scientific) which was supplemented with protease and phosphatase inhibitors. The resulting cell suspensions were transferred to pre-chilled microcentrifuge tubes, incubated on ice for 20 min with intermittent mixing and then centrifuged at 12,000 rpm for 20 min at 4° C. The supernatants were then aspirated slowly and transferred into new tubes kept on ice. The protein concentration in the samples was determined using BCA kit (Thermo Fisher Scientific) as per manufacturer's instructions.

For the p62 oligomerization and LC3 lipidation assays, cell lysates were prepared using RIPA buffer supplemented with 2% SDS and benzonase (Sigma). The lysates were then combined with 4× Laemmli protein sample buffer (Bio-Rad), heated at 70° C. for 10 min and subjected to a brief centrifugation of 10-15 seconds.

For the BRD4 degradation assay, cell lysates were prepared with RIPA buffer containing 0.25% SDS. These were mixed with 4× Laemmli protein sample buffer (Bio-Rad) supplemented with beta-mercaptoethaol (355 mM). The lysates were then heated at 95° C. for 5 min, and briefly centrifuged for 10-15 seconds.

For the FKBP12(F36V) degradation assay, cell lysates were prepared with a Triton-X-100 buffer supplemented with 2% SDS. The lysates were mixed with 4× Laemmli protein sample buffer (Bio-Rad) supplemented with beta-mercaptoethaol (355 mM). The lysates were then heated at 95° C. for 5 min, and briefly centrifuged for 10-15 seconds.

For p62 oligomerization and LC3 lipidation assays, lysates containing 30 μg of protein and for BRD4 and FKBP12(F36V) degradation assays, lysates containing 20 μg of protein, were loaded slowly into the wells of 4-20% gradient Tris-glycine gels (Bio-Rad). The gels were run at 100V until completion and transferred onto PVDF membranes. Membranes were blocked using 5% milk in TBS-Tween (0.1%) buffer at room temperature for 1 hour. The membranes were incubated with primary antibody in 5% milk-TBST with shaking, overnight at 4° C. The primary antibodies used were: p62 (Novus Biologicals 2C11, H00008878-MO1), LC3A/B (Cell Signalling Technology D3U4C, 12741S), FKBP12 (Abcam ab24373), BRD4 (Cell Signalling Technology E2A7X, 13440S), GAPDH (Cell Signalling Technology D4C6R, 97166S), and β-Actin (Proteintech 66009-1-Ig). Following primary antibody incubation, membranes were washed thrice with TBST 10 min before and after incubation with secondary-HRP conjugated antibody in 5% milk-TBST for 1 hour at room temperature. The secondary antibodies used were: Anti-Mouse IgG HRP Conjugate (Promega W4021B) and Anti-Rabbit IgG HRP Conjugate (Promega W4011B). For the p62 oligomerization assay, the total protein levels were assessed using Ponceau staining (Thermo Fisher Scientific) of PVDF membrane as per recommendation by manufacturer's guidelines.

Imaging was acquired using SuperSignal™ West Pico PLUS Chemiluminescent Substrate (Thermo Fisher Scientific) with iBright Invitrogen FL100 imaging system. Band signal intensity, reflective of protein concentrations, were quantified using Invitrogen™ iBright™ Analysis and Image J software.

### Example 30: Immunocytochemistry and Imaging Protocol

HeLa cells were seeded in coated 96-well plates (10,000 cells) and allowed to adhere overnight. After 24 hours of treatment with either vehicle (DMSO) or test compounds, the media was discarded, and cells were rinsed with ice-cold 1×PBS before fixation by 4% formaldehyde or paraformaldehyde for 20 min. Cells were then washed with 1×PBS and permeabilized with 0.1% Triton X-100 in PBS for 20 min and blocked with Blocking Buffer in PBS for 1 hour. The cells were incubated thereafter for 1 hour with anti-p62 primary antibody in Blocking Buffer, followed by incubation with secondary antibody for 1 hour. DAPI dye was used to visualize nuclei of fixed cells. The blocking buffer used were 5% Fetal Bovine Serum (FBS, ThermoFIsher Scientific, 10270106) and Li-cor Intercept (PBS) Blocking Buffer (Li-cor, 927-70001). The primary antibody used was mouse anti-p62 primary antibody (Novus Biologicals, H00008878-MO1). The secondary antibodies used were goat anti-mouse Alexa Fluor Plus 488 secondary antibody (Invitrogen, GOXMS) and goat anti-mouse Alexa Fluor 488 secondary antibody (Invitrogen, A-11029). The DAPI dyes used were DAPI dye (Invitrogen, 62248) and DAPI dye (Invitrogen, D1306).

For the EVOS M7000 System, cells were imaged on the EVOS M7000 microscope using a 20× air objective (Nikon, N.A 0.45). DAPI and p62 channels were illuminated with LED light cubes using integrated hard-coated filter sets for DAPI (357/447 nm) and GFP (470/525 nm) respectively. Images were analysed for p62 puncta count on the Celeste 6.0 software.

For the Operetta CLS System, cells were imaged on the Operetta CLS using a 63× water objective on the DAPI and Alexa 488 channels. Images were analysed for p62 puncta count on the Harmony 5.1 software.

### Example 31: Studies on Compound Binding to p62

Compound binding studies were carried out according to experimental procedures above and SPR % occupancy was determined and summarized in Table 7.

TABLE 7
SPR assay data (% occupancy at 100 μM)

| | SPR % |
|---|---|
| Cpd No. | occupancy |
| 1 | + |
| 2 | ++ |
| 3 | ++ |
| 4 | ++ |
| 5 | ++ |
| 6 | + |
| 7 | ++ |
| 8 | ++ |
| 9 | +++ |
| 10 | ++ |
| 11 | + |
| 12 | +++ |
| 13 | +++ |
| 15 | +++ |
| 16 | ++ |
| 17 | ++ |
| 18 | ++ |
| 19 | ++ |
| 23 | + |
| 28 | + |
| 29 | + |
| 32 | + |
| 33 | ++ |
| 34 | + |
| 35 | ++ |
| 36 | ++ |
| 37 | ++ |
| 38 | ++ |
| 42 | ++ |
| 43 | ++ |
| 45 | ++ |
| 47 | + |
| 48 | ++ |
| 49 | ++ |
| 50 | + |

Legends:
+: % occupancy <30
++: % occupancy 30-70
+++: % occupancy >70

### Example 32: Oligomerization Activity of p62 Protein

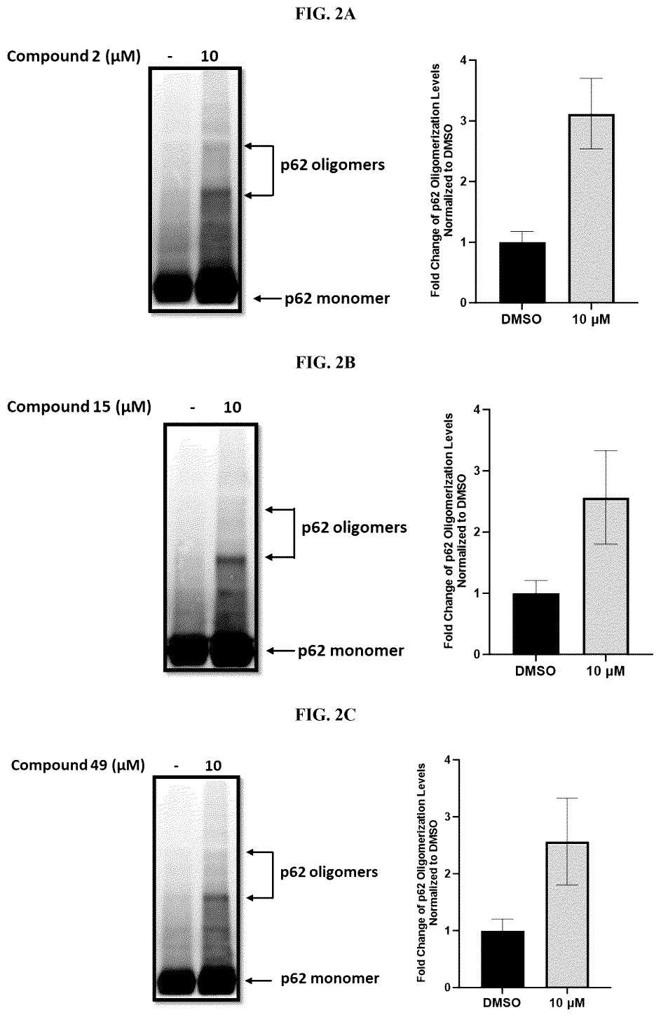

HeLa cells were treated with compounds for 24 hours to evaluate the effect of these compounds on p62 oligomerisation activity. The resulting changes in levels of p62 oligomers were assessed by Western blotting, as described in experimental procedures. The fold change of p62 oligomer levels induced by compound treatment relative to vehicle control (DMSO) was determined and summarized in Table 8. Representative Western blot images are shown in FIGS. 2A-C. Specifically, FIGS. 2A-C show Western blot images and their corresponding quantification showing the levels of high molecular weight (100-200 kDa) p62 oligomers by 24 hours compound treatments at listed concentrations, relative to vehicle control (DMSO) in HeLa cells. Protein loading consistencies was assessed by Ponceau staining (not shown).

TABLE 8
Compound effect on p62 oligomerization

| Cpd No. | p62 oligomerization |
|---|---|
| 1 | + |
| 2 | ++ |
| 3 | ++ |
| 5 | + |
| 7 | ++ |
| 8 | ++ |
| 9 | + |
| 10 | ++ |
| 12 | ++ |
| 13 | ++ |
| 15 | ++ |
| 17 | + |
| 33 | + |
| 35 | + |
| 37 | ++ |
| 42 | + |
| 43 | + |
| 49 | ++ |

Legend:
++: > 2-fold change relative to DMSO control
+: < 2-fold change relative to DMSO control

### Example 33: Lipidation Activity of LC3 Protein

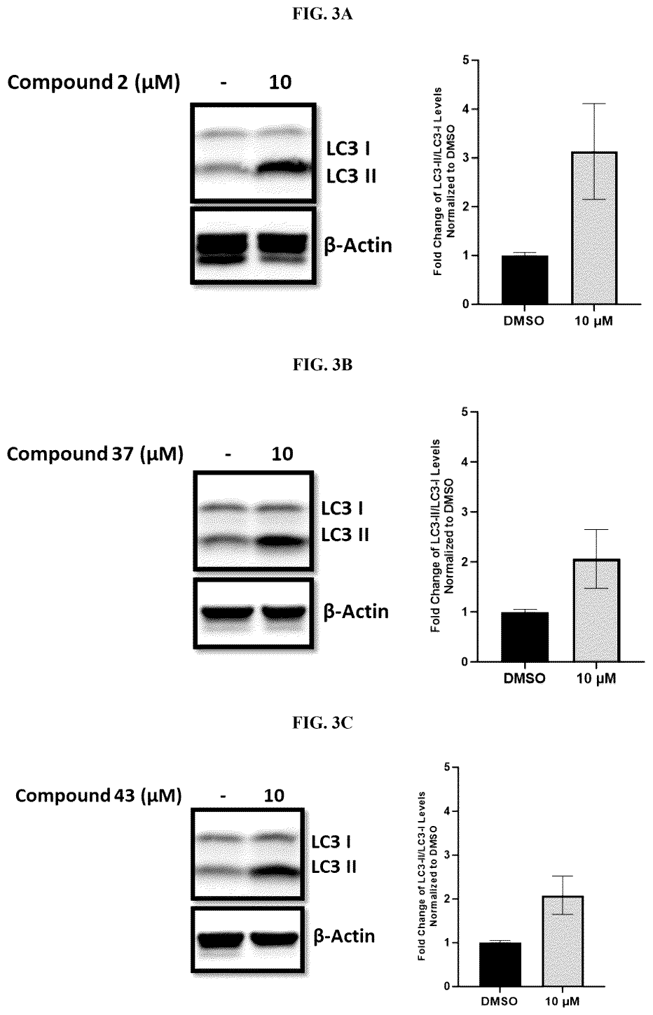

HeLa cells were treated with compounds for 24 hours to evaluate the effect of these compounds on LC3-II/LC3-I levels, a measure of autophagy activation. The changes in levels of LC3-I, LC3-II and β-Actin (loading control) were assessed by Western blotting, as described in experimental procedures. The fold increase of LC3-II/LC3-I levels induced by compound treatment relative to vehicle control (DMSO) was determined and summarized in Table 9. Representative Western blot images are shown in FIGS. 3A-C. Specifically, FIGS. 3A-C show Western blot images and their corresponding quantification showing the lipidation levels of LC3 protein (LC3-II/LC3-I) by 24 hours compound treatments at listed concentrations, relative to vehicle control (DMSO) in HeLa cells.

TABLE 9
Compound effect on LC3 lipidation

| Cpd No. | LC3 lipidation |
|---|---|
| 1 | + |
| 2 | ++ |
| 3 | ++ |
| 5 | + |
| 7 | ++ |
| 8 | + |
| 9 | ++ |
| 10 | + |
| 12 | + |
| 13 | ++ |
| 15 | + |
| 17 | + |
| 33 | + |
| 35 | + |
| 37 | ++ |
| 42 | + |
| 43 | ++ |
| 49 | + |

Legend:
++: > 2-fold change in LC3-II/LC3-I relative to DMSO control
+: < 2-fold change in LC3-II/LC3-I relative to DMSO control

### Example 34: Puncta Activity of p62 Protein

HeLa cells were treated with compounds for 24 hours to evaluate the effect of these compounds on p62 puncta activity. The resulting changes in levels of p62 puncta were assessed by imaging, either on the EVOS M7000 or Operetta CLS systems as described in experimental procedures. The fold change of p62 puncta levels induced by compound treatment relative to vehicle control (DMSO) was determined and summarized in Table 10.

TABLE 10
Compound effect on p62 puncta (HeLa, 24 hr, 5 μM)

| Cpd No. | p62 puncta |
|---|---|
| 1 | ++ |
| 2 | ++ |
| 12 | ++ |
| 13 | ++ |
| 25 | + |
| 42 | + |
| 45 | + |
| 49 | ++ |
| 72 | ++ |
| 73 | ++ |
| 74 | ++ |
| 76 | ++ |
| 77 | ++ |
| 78 | ++ |
| 79 | ++ |
| 80 | ++ |
| 82 | + |
| 83 | ++ |
| 84 | ++ |
| 86 | ++ |
| 87 | ++ |
| 90 | ++ |
| 94 | ++ |
| 95 | ++ |
| 96 | ++ |
| 98 | ++ |
| 99 | + |
| 105 | ++ |
| 106 | ++ |
| 107 | + |
| 108 | ++ |
| 109 | + |
| 110 | + |
| 112 | + |
| 113 | ++ |
| 120 | ++ |
| 121 | + |
| 122 | ++ |
| 123 | ++ |
| 124 | ++ |
| 125 | ++ |
| 126 | ++ |
| 127 | ++ |
| 128 | ++ |
| 129 | ++ |
| 131 | ++ |
| 132 | ++ |
| 133 | ++ |
| 134 | ++ |
| 135 | + |
| 136 | ++ |
| 137 | + |
| 138 | ++ |
| 139 | + |
| 140 | + |
| 141 | ++ |
| 142 | ++ |
| 145 | + |
| 146 | + |
| 147 | ++ |
| 148 | + |
| 149 | ++ |
| 151 | ++ |
| 152 | ++ |
| 153 | ++ |
| 154 | ++ |
| 155 | ++ |
| 156 | ++ |
| 157 | ++ |
| 158 | ++ |
| 159 | ++ |
| 160 | + |
| 161 | ++ |
| 162 | ++ |
| 163 | ++ |
| 164 | ++ |
| 165 | ++ |
| 166 | ++ |
| 167 | ++ |
| 168 | ++ |

Legend:
++: > 2-fold change in p62 puncta levels relative to DMSO control
+: < 2-fold change in p62 puncta levels relative to DMSO control

### Example 35: Degradation of BRD4

To evaluate levels of protein degradation by compounds 100-103, HeLa cells were treated with the corresponding compounds at doses up to 10 μM for 24 hours. The changes in levels of BRD4 and GAPDH (loading control) were assessed by Western blotting, as described in experimental procedures. Results are summarized in Table 11 below, measured by percentage change in BRD4 levels by compound treatment relative to vehicle (DMSO) control.

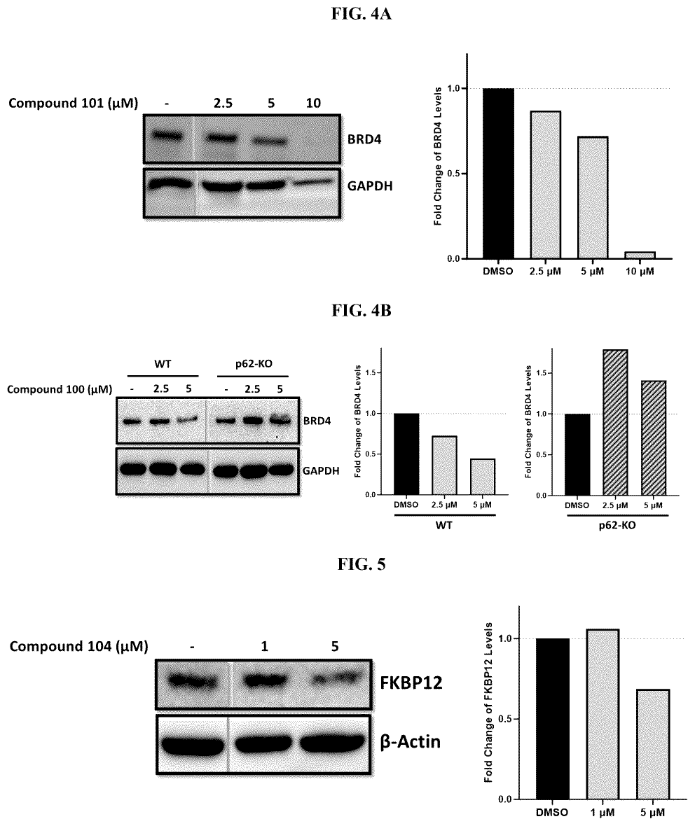

To demonstrate p62 dependency of the compounds, wild type (WT) and p62 knockout (p62-KO) HeLa cells were treated with respective compounds for 24 hours. Compound-mediated BRD4 degradation was not observed in p62-KO cells in contrast to WT HeLa cells. Representative Western blot images are shown in FIGS. 4A-B. Specifically, FIGS. 4A-B show Western blot images and their corresponding quantifications showing the degradation levels of BRD4 by 24 hours compound treatments at listed concentrations, relative to vehicle control (DMSO) in wild type (WT) and/or p62 knockout (p62-KO) HeLa cells.

TABLE 11
BRD4 degradation by compounds 100-103

| Cpd No. | Degradation |
|---|---|
| 100 | ++ |
| 101 | ++ |
| 102 | + |
| 103 | ++ |

Legend:
++: > 2-fold change in BRD4 levels relative to DMSO control
+: < 2-fold change in BRD4 levels relative to DMSO control

### Example 36: Degradation of FKBP12(F36V)

HeLa cells expressing FKBP12-F36V were treated with compound 104 for 24 hours to evaluate its ability to degrade the target protein. The changes in levels of FKBP12 and 0-Actin (loading control) were assessed by Western blotting, as described in experimental procedures. Representative Western blots images are shown in FIG. 5. Specifically, FIG. 5 shows Western blot images and their corresponding quantification showing the degradation levels of FKBP12 by 24 hours compound treatment at listed concentrations, relative to vehicle control (DMSO) in HeLa cells expressing FKBP12(F36V).

The publications discussed herein are provided solely for their disclosure prior to the filing date of the present application. Nothing herein is to be construed as an admission that the present invention is not entitled to antedate such publication by virtue of prior invention.

While the invention has been described in connection with proposed specific embodiments thereof, it will be understood that it is capable of further modifications and this application is intended to cover any variations, uses, or adaptations of the invention following, in general, the principles of the invention and including such departures from the present disclosure as come within known or customary practice within the art to which the invention pertains and as may be applied to the essential features hereinbefore set forth and as follows in the scope of the appended claims.

1. A compound of formula (I) 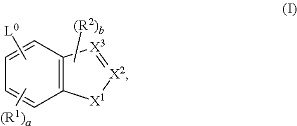
or a pharmaceutically acceptable salt, stereoisomer, or deuterated form thereof,
wherein:
L⁰ is —C₁₋₆ alkylene-NR^{A}R^{B}, —C₁₋₆ alkylene-NR^{A}—C₁₋₆ alkylene-R^{B}, —C₁₋₆ alkylene-NR^{A}—C₃₋₈ cycloalkylene-R^{B}, —C₁₋₆ alkylene-NR^{A}C(O)NR^{A}R^{B}, —C₁₋₆ alkylene-NR^{A}C(O)—R^{B}, —C₁₋₆ alkylene-O—C₁₋₆ alkylene-NR^{A}R^{B}, —C₁₋₆ alkylene-O—C₁₋₆ alkylene-C(O)NR^{A}R^{B}, —C₁₋₆ alkylene-O—C₁₋₆ alkylene-NR^{A}C(O)—R^{B}, —C₁₋₆ alkylene-heterocyclylene-O—C₁₋₆ alkyl, —C₃₋₈ cycloalkylene-NR^{A}R^{B}, —C(O)NR^{A}R^{B}, —O—C₁₋₆ alkylene-C(O)NR^{A}R^{B}, —O—C₁₋₆ alkylene-NR^{A}R^{B}, —NR^{A}C(O)R^{B}, —NR^{A}C(O)NR^{A}R^{B}, or —NR^{A}C(O)NR^{A}—C₁₋₆ alkylene-R^{B}, wherein the alkylene is optionally substituted with —OH or halogen;
each R¹ is independently C₁₋₆ alkyl, C₃-C₈ cycloalkyl, or halogen;
each R² is independently C₁₋₆ alkyl, C₃-C₈ cycloalkyl, or halogen;
X¹ is O or S;
one of X² or X³ is C-Q-R³ and the other is CH, C, or N as permitted by valency;
Q is absent, C₁₋₆ alkylene, —C₁₋₆ alkylene-C(O)—, C₃₋₈ cycloalkylene, —C₃₋₈ cycloalkylene-C(O)—, —C(O)—, or —S(O)₂—;
R³ is carbocycle, heterocycle, aryl, heteroaryl, or —NR^{X}R^{Y}, wherein the carbocycle, heterocycle, aryl, and heteroaryl are each optionally substituted with 1, 2, or 3 R⁴;
each R⁴ is independently halogen, —OH, C₁₋₆ alkyl, C₁₋₆ alkoxy, —C(O)—NR^{X}R^{Y}, or —C(O)O—(C₁₋₆ alkyl);
each R^{A} is independently H, C₁₋₆ alkyl, C₁₋₆ haloalkyl, C₃₋₈ cycloalkyl, C₃₋₈ hydroxycycloalkyl, aryl, or heterocycle;
each R^{B} is independently C₁₋₆ alkyl, C₁₋₆ hydroxyalkyl, C₁₋₆ alkoxy, C₃₋₈ hydroxycycloalkyl, C₁₋₆ alkylene-NH₂, or C₁₋₆ alkylene-SH;
R^{X} is H, C₁₋₆ alkyl, C₁₋₆ haloalkyl, or C₁₋₆ hydroxyalkyl;
R^{Y} is C₁₋₆ alkyl, C₃₋₈ cycloalkyl, C₃₋₈ hydroxycycloalkyl, aryl, or heterocycle;
a is an integer of 0-3;
b is an integer of 0 or 1; and
the compound of formula (I) is not

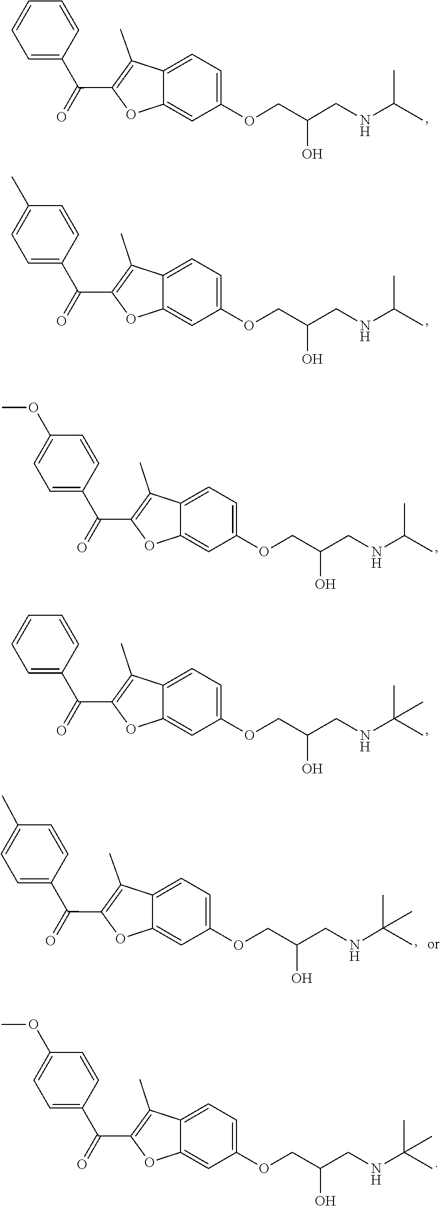

2. The compound of claim 1, having a structure of formula (I-A): 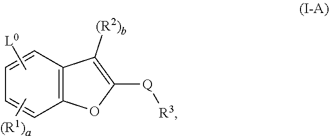
or a pharmaceutically acceptable salt, a stereoisomer, or a deuterated form thereof.

3. The compound of claim 1, having a structure of formula (I-B): 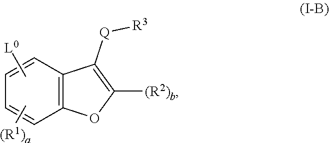
or a pharmaceutically acceptable salt, a stereoisomer, or a deuterated form thereof.

4. The compound of claim 1, having a structure of formula (I-C) or (I-D): 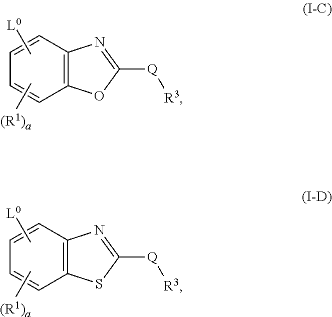
or a pharmaceutically acceptable salt, a stereoisomer, or a deuterated form thereof.

5. The compound of claim 1, having a structure of formula (I-A-1), (I-A-2), (I-A-3), or (I-A-4): 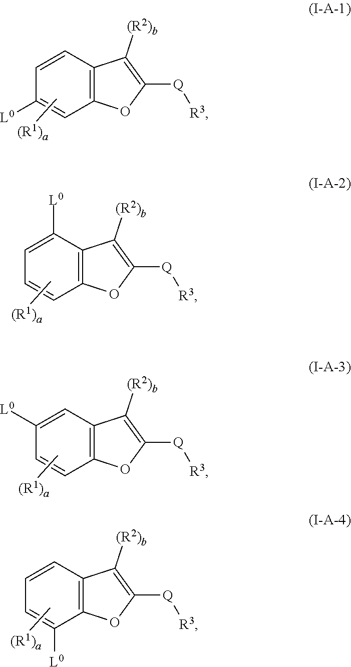
or a pharmaceutically acceptable salt, a stereoisomer, or a deuterated form thereof.

6. The compound of claim 1, having a structure of formula (I-B-1), (I-B-2), (I-B-3), or (I-B-4): 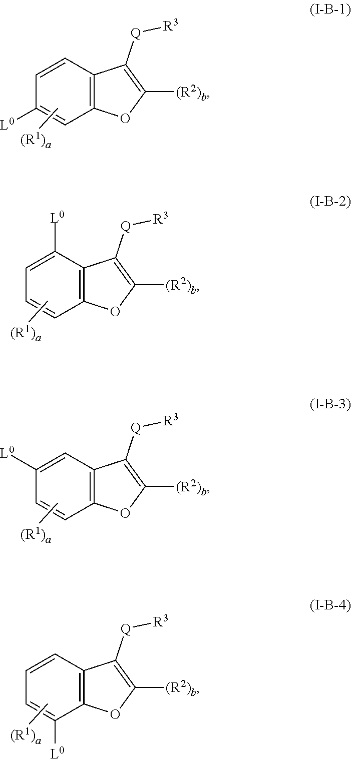
or a pharmaceutically acceptable salt, a stereoisomer, or a deuterated form thereof.

7. The compound of claim 1, having a structure of formula (I-C-1), (I-C-2), (I-C-3), (I-C-4), (I-D-1), (I-D-2), (I-D-3), or (I-D-4): 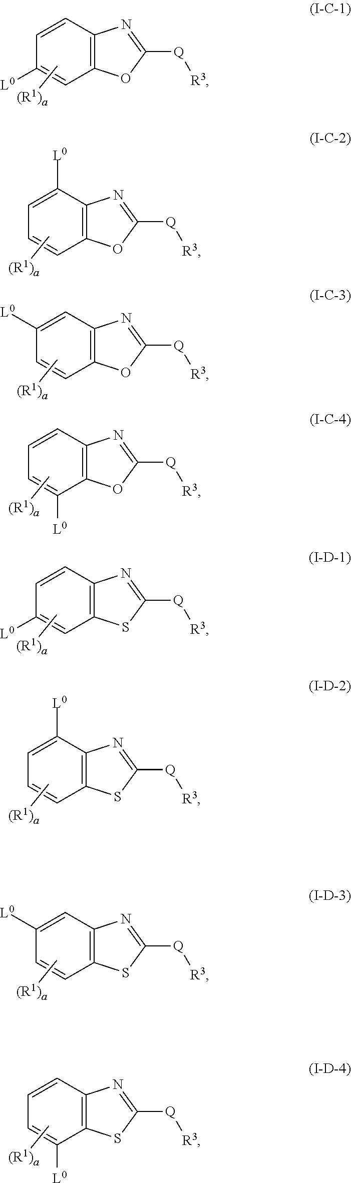
or a pharmaceutically acceptable salt, a stereoisomer, or a deuterated form thereof.

8. The compound of claim 1, or a pharmaceutically acceptable salt, stereoisomer, or deuterated form thereof, wherein L⁰ is —(C₁₋₆ alkylene)-N(H)(C₁₋₆ hydroxyalkyl), —(C₁₋₆ alkylene)-N(C₁₋₆ alkyl)(C₁₋₆ hydroxyalkyl), —(C₁₋₆ alkylene)-N(H)(C₁₋₆ alkoxy), —(C₁₋₆ alkylene)-N(H)(C₁₋₆ alkylene)(C₁₋₆ alkoxy), —C(O)N(H)(C₁₋₆ hydroxyalkyl), —(C₁₋₆ alkylene)-N(H)(C₃₋₈ cycloalkylene)(C₁₋₆ alkoxy), —(C₁₋₆ alkylene)-N(C₁₋₆ alkyl)(C₁₋₆ alkoxy), —(C₁₋₆ alkylene)-N(H)C(O)(C₁₋₆ alkoxy), —(C₁₋₆ alkylene)-N(C₁₋₆ alkyl)C(O)(C₁₋₆ alkoxy), —(C₁₋₆ alkylene)-N(H)C(O)N(H)(C₁₋₆ alkoxy), —(C₁₋₆ alkylene)-N(C₁₋₆ alkyl)C(O)N(C₁₋₆ alkyl)(C₁₋₆ alkoxy), —(C₃₋₈ cycloalkylene)N(H)(C₁₋₆ hydroxyalkyl), —(C₃₋₈ cycloalkylene)N(C₁₋₆ alkyl)(C₁₋₆ hydroxyalkyl), —O—(C₁₋₆ alkylene)-N(H)(C₁₋₆ hydroxyalkyl), —O—(C₁₋₆ alkylene)-N(C₁₋₆ alkyl)(C₁₋₆ hydroxyalkyl), —N(H)(C₁₋₆ hydroxyalkyl), —N(H)C(O)N(H)(C₁₋₆ hydroxyalkyl), —N(H)C(O)N(H)(C₁₋₆ alkylene)-(C₁₋₆ alkoxy), —(C₁₋₆ alkylene)-O—(C₁₋₆ alkylene)C(O)N(H)(C₁₋₆ alkyl), —(C₁₋₆ alkylene)-O—(C₁₋₆ alkylene)N(H)C(O)(C₁₋₆ alkyl), —O—(C₁₋₆ alkylene)C(O)N(H)(C₁₋₆ alkyl), —N(C₁₋₆ alkyl)(C₁₋₆ hydroxyalkyl), —N(H)C(O)(C₁₋₆ hydroxyalkyl), —N(C₁₋₆ alkyl)C(O)(C₁₋₆ hydroxyalkyl), —(C₁₋₆ alkylene)-heterocyclylene-(C₁₋₆ alkoxy), —(C₁₋₆ alkylene)-O—(C₁₋₆ alkylene)-N(H)(C₁₋₆ alkyl), or —(C₁₋₆ alkylene)-O—(C₁₋₆ alkylene)-N(C₁₋₆ alkyl)(C₁₋₆ alkyl),
wherein the alkylene or alkyl is optionally substituted with —OH or halogen.

9. The compound of claim 1, or a pharmaceutically acceptable salt, stereoisomer, or deuterated form thereof, wherein L⁰ is
—CH₂NHCH₂CH₂OH,
—CH₂NHCH₂CH(CH₃)OH,
—CH(CH₃)NHCH₂CH₂OH, 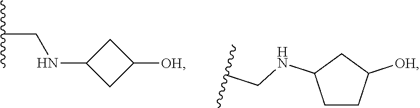 [SMILES: *CNC1CC(O)C1 ; *CNC1CCC(O)C1]
—CH₂NHCH₂CH₂OCH₃,
—CH₂NHCH₂CH₂OCH(CH₃)₂, 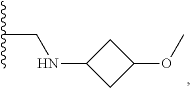 [SMILES: *CNC1CC(OC)C1]
—CH₂OCH₂CH₂NHCH₃,
—CH₂OCH₂CH₂NHCH(CH₃)₂,
—CH₂OCH₂CH₂N(CH₃)₂,
—C(O)NHCH₂CH₂OH,
—NHC(O)CH₂CH₂OH,
—NHC(O)CH₂OH,
—CH₂NHC(O)NHCH₂CH₂OH, 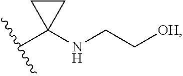 [SMILES: *C1(NCCO)CC1]
—NHC(O)NHCH₂CH₂OH,
—OCH₂CH(OH)CH₂NHCH(CH₃)₂,
—OCH₂CH₂C(O)NHCH₃,
—CH₂OCH₂CH₂C(O)NHCH₃,
—CH₂OCH₂CH₂NHC(O)CH₃,
—CH₂NHC(O)CH₂OH,
—CH₂NHC(O)CH₂CH₂OH, or
—NHC(O)NHCH₂CH₂OCH₃,

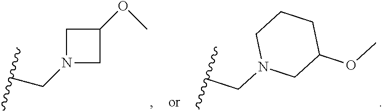

10. The compound of claim 1, or a pharmaceutically acceptable salt, stereoisomer, or deuterated form thereof, wherein L⁰ is —CH₂NHCH₂CH₂OH, —CH₂NHCH₂CH(CH₃)OH, —CH₂OCH₂CH₂NHCH₃, —OCH₂CH(OH)CH₂NHCH(CH₃)₂, —CH₂OCH₂CH₂NHCH(CH₃)₂, or

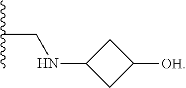 [SMILES: *CNC1CC(O)C1]

11. The compound of claim 1, having a structure of formula (I-A-1-a), (I-A-2-a), or (I-A-3-a): 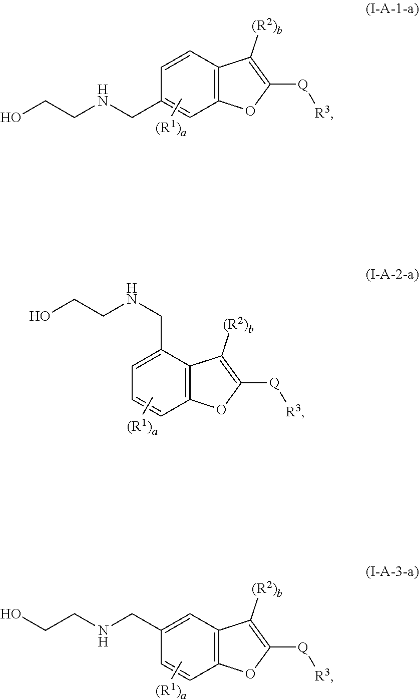
or a pharmaceutically acceptable salt, a stereoisomer, or a deuterated form thereof.

12. The compound of claim 1, having a structure of formula (I-B-1-a), (I-B-2-a), or (I-B-3-a): 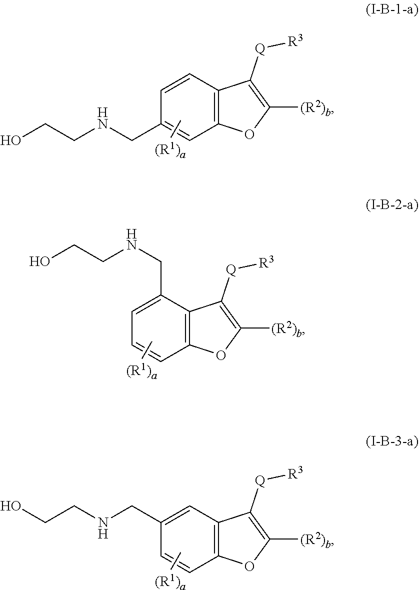
or a pharmaceutically acceptable salt, a stereoisomer, or a deuterated form thereof.

13. The compound of claim 1, having a structure of formula (I-C-1-a) or (I-D-1-a): 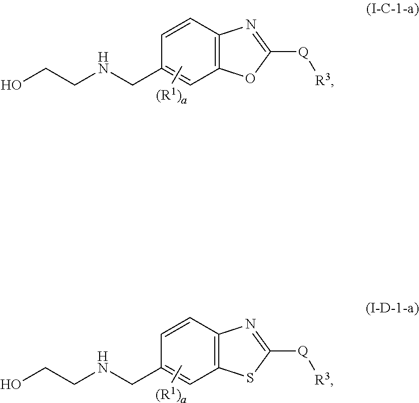
or a pharmaceutically acceptable salt, a stereoisomer, or a deuterated form thereof.

14. The compound of claim 1, wherein at least one H in L⁰ is replaced by conjugate comprising a ligand that binds to a protein, a protein aggregate, a protein complex, or a lipid.

15. A compound of formula (II) 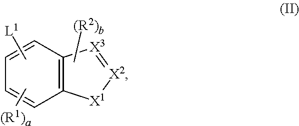
or a pharmaceutically acceptable salt, stereoisomer, or deuterated form thereof,
wherein:
L¹ is C₂-C₅₀ alkylene-R⁵, C₂-C₂₅ alkenylene-R⁵, C₂-C₂₅ alkynylene-R⁵, wherein 1-25 methylene groups of L¹ are optionally and independently replaced by —N(H)—, —N(C₁-C₆ alkyl)-, —N(C₃-C₈ cycloalkyl)-, —O—, —C(O)—, —C(O)O—, —S—, —S(O)—, —S(O)₂—, —S(O)₂N(C₁-C₆ alkyl)-, —S(O)₂N(C₃-C₈ cycloalkyl)-, —N(H)C(O)—, —N(C₁-C₆ alkyl)C(O)—, —N(C₃-C₈ cycloalkyl)C(O)—, —N(H)C(O)N(H)—, —N(C₁-C₆ alkyl)C(O)N(H)—, —N(H)C(O)N(C₁-C₆ alkyl)-, —N(C₁-C₆ alkyl)C(O)N(C₁-C₆ alkyl)-, —C(O)N(H)—, —C(O)N(C₁-C₆ alkyl)-, —C(O)N(C₃-C₈ cycloalkyl)-, arylene, heteroarylene, heterocyclylene, C₃-C₈ cycloalkylene, or C₃-C₈ cycloalkenylene, wherein the alkylene, alkenylene, alkynylene, alkyl, arylene, heteroarylene, and heterocyclylene are each optionally and independently substituted with 1, 2, or 3 R^{Z}, wherein each R^{Z} is independently —OH, C₁₋₆ alkyl, or halogen;
each R¹ is independently C₁₋₆ alkyl, C₃-C₈ cycloalkyl, or halogen;
each R² is independently C₁₋₆ alkyl, C₃-C₈ cycloalkyl, or halogen;
X¹ is O or S;
one of X² or X³ is C-Q-R³ and the other is CH, C, or N as permitted by valency;
Q is absent, C₁₋₆ alkylene, —C₁₋₆ alkylene-C(O)—, C₃₋₈ cycloalkylene, —C₃₋₈ cycloalkylene-C(O)—, —C(O)—, or —S(O)₂—;
R³ is carbocycle, heterocycle, aryl, heteroaryl, or —NR^{X}R^{Y}, wherein the carbocycle, heterocycle, aryl, and heteroaryl are each optionally substituted with 1, 2, or 3 R⁴;
each R⁴ is independently halogen, —OH, C₁₋₆ alkyl, C₁₋₆ alkoxy, —C(O)—NR^{X}R^{Y}, or —C(O)O—(C₁₋₆ alkyl);
each R⁵ is independently a bond, a leaving group, a protecting group, H, alkynyl, aryl, or heteroaryl;
R^{X} is H, C₁₋₆ alkyl, C₁₋₆ haloalkyl, or C₁₋₆ hydroxyalkyl;
R^{Y} is C₁₋₆ alkyl, C₃₋₈ cycloalkyl, C₃₋₈ hydroxycycloalkyl, aryl, or heterocycle;
a is an integer of 0-3;
b is an integer of 0 or 1; and
the compound of formula (II) is not

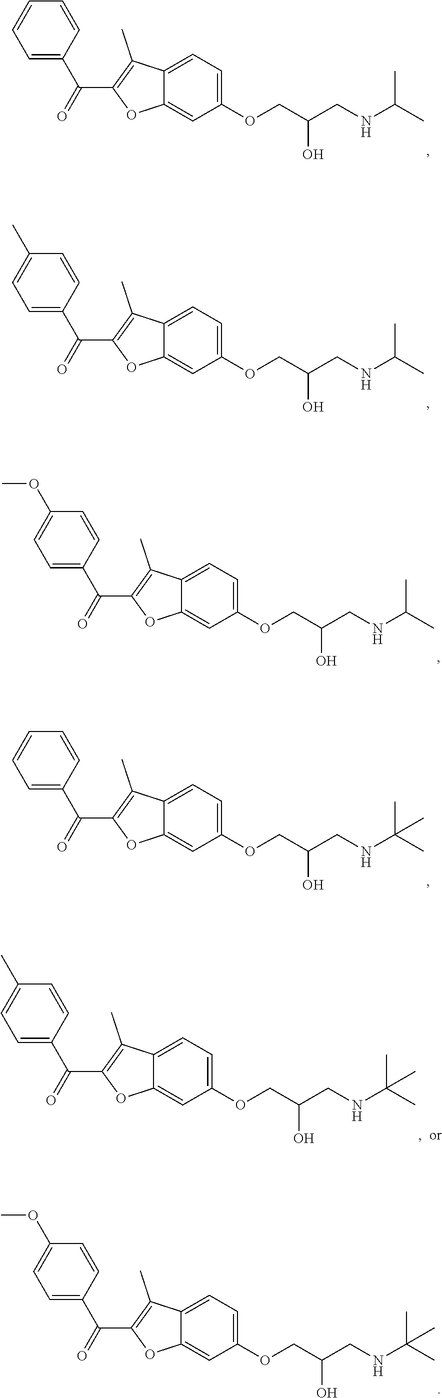

16.-29. (canceled)

30. A compound of formula (III) 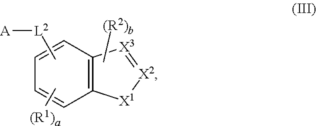
or a pharmaceutically acceptable salt, stereoisomer, or deuterated form thereof,
wherein:
A is a ligand that binds to a protein, a protein aggregate, a protein complex, or a lipid;
L² is C₂-C₅₀ alkylene, C₂-C₂₅ alkenylene, C₂-C₂₅ alkynylene, or —(C₂-C₅₀ alkylene)-arylene, wherein 1-25 methylene groups of L² are optionally and independently replaced by —N(H)—, —N(C₁-C₆ alkyl)-, —N(C₃-C₈ cycloalkyl)-, —O—, —C(O)—, —C(O)O—, —S—, —S(O)—, —S(O)₂—, —S(O)₂N(C₁-C₆ alkyl)-, —S(O)₂N(C₃-C₈ cycloalkyl)-, —N(H)C(O)—, —N(C₁-C₆ alkyl)C(O)—, —N(C₃-C₈ cycloalkyl)C(O)—, —N(H)C(O)N(H)—, —N(C₁-C₆ alkyl)C(O)N(H)—, —N(H)C(O)N(C₁-C₆ alkyl)-, —N(C₁-C₆ alkyl)C(O)N(C₁-C₆ alkyl)-, —C(O)N(H)—, —C(O)N(C₁-C₆ alkyl)-, —C(O)N(C₃-C₈ cycloalkyl)-, arylene, heteroarylene, heterocyclylene, C₃-C₈ cycloalkylene, or C₃-C₈ cycloalkenylene, wherein the alkylene, alkenylene, alkynylene, alkyl, arylene, heteroarylene, and heterocyclylene are each optionally and independently substituted with 1, 2, or 3 R^{Z}, wherein each R^{Z} is independently OH, C₁₋₆ alkyl, or halogen;
each R¹ is independently C₁₋₆ alkyl, C₃-C₈ cycloalkyl, or halogen;
each R² is independently C₁₋₆ alkyl, C₃-C₈ cycloalkyl, or halogen;
X¹ is O or S;
one of X² or X³ is C-Q-R³ and the other is CH, C, or N as permitted by valency;
Q is absent, C₁₋₆ alkylene, —C₁₋₆ alkylene-C(O)—, C₃₋₈ cycloalkylene, —C₃₋₈ cycloalkylene-C(O)—, —C(O)—, or —S(O)₂—;
R³ is carbocycle, heterocycle, aryl, heteroaryl, or —NR^{X}R^{Y}, wherein the carbocycle, heterocycle, aryl, and heteroaryl are each optionally substituted with 1, 2, or 3 R⁴;
each R⁴ is independently halogen, —OH, C₁₋₆ alkyl, C₁₋₆ alkoxy, —C(O)—NR^{X}R^{Y}, or —C(O)O—(C₁₋₆ alkyl);
R^{X} is H, C₁₋₆ alkyl, C₁₋₆ haloalkyl, or C₁₋₆ hydroxyalkyl;
R^{Y} is C₁₋₆ alkyl, C₃₋₈ cycloalkyl, C₃₋₈ hydroxycycloalkyl, aryl, or heterocycle;
a is an integer of 0-3; and
b is an integer of 0 or 1.

31.-40. (canceled)

41. The compound of claim 30, or a pharmaceutically acceptable salt, a stereoisomer, or a deuterated form thereof, wherein:
the protein is a protein that is associated with cancer, optionally wherein the protein associated with cancer comprises a mutation or a fusion, and optionally wherein the protein associated with cancer is BRD4;
the protein is a protein associated with a metabolic disease;
the protein is a protein associated with inflammation;
the protein is present in bacteria;
the protein is present in a virus particle;
the protein is a mitochondrial protein;
the protein is an intracellular protein; or
the protein aggregate is a Tau protein aggregate, an alpha-synuclein protein aggregate, a β-sheet protein aggregate, a mutant Huntingtin protein aggregate, an amyloid protein aggregate, or a TDP-43 protein aggregate.

42.-52. (canceled)

53. The compound of claim 1, or a pharmaceutically acceptable salt, a stereoisomer, or a deuterated form thereof, wherein each R¹ is independent halogen.

54. (canceled)

55. The compound of claim 1, or a pharmaceutically acceptable salt, a stereoisomer, or a deuterated form thereof, wherein each R¹ is independently C₁₋₆ alkyl.

56.-57. (canceled)

58. The compound of claim 1, or a pharmaceutically acceptable salt, a stereoisomer, or a deuterated form thereof, wherein a is 0 or 1.

59. The compound of claim 1, or a pharmaceutically acceptable salt, a stereoisomer, or a deuterated form thereof, wherein each R² is independently C₁₋₆ alkyl.

60.-61. (canceled)

62. The compound of claim 1, or a pharmaceutically acceptable salt, a stereoisomer, or a deuterated form thereof, wherein b is 0 or 1.

63. The compound of claim 1, or a pharmaceutically acceptable salt, a stereoisomer, or a deuterated form thereof, wherein Q is absent, C₁₋₄ alkylene, —(C₁₋₃ alkylene)C(O)—, C₃₋₆ cycloalkylene, —(C₃₋₆ cycloalkylene)C(O)—, —C(O)—, or —S(O)₂—.

64.-72. (canceled)

73. The compound of claim 1, or a pharmaceutically acceptable salt, a stereoisomer, or a deuterated form thereof, wherein R³ is C₃₋₈ carbocycle, 3-10 membered heterocycle, C₆₋₁₀ aryl, 5-10 membered heteroaryl, or —NR^{X}R^{Y}, wherein the C₃₋₈ carbocycle, 3-8 membered heterocycle, C₆₋₁₀ aryl, and 5-10 membered heteroaryl are each optionally substituted with 1 or 2 R⁴, and each R⁴ is independently halogen, —OH, C₁₋₆ alkyl, C₁₋₆ alkoxy, —C(O)—NR^{X}R^{Y}, or —C(O)O—C₁₋₆ alkyl, and wherein R^{X} is H, C₁₋₆ alkyl, C₁₋₆ haloalkyl, or C₁₋₆ hydroxyalkyl, and R^{Y} is C₁₋₆ alkyl, C₃₋₈ cycloalkyl, C₃₋₈ hydroxycycloalkyl, aryl, or heterocycle.

74.-94. (canceled)

95. The compound of claim 1, wherein the compound of formula (I) is: 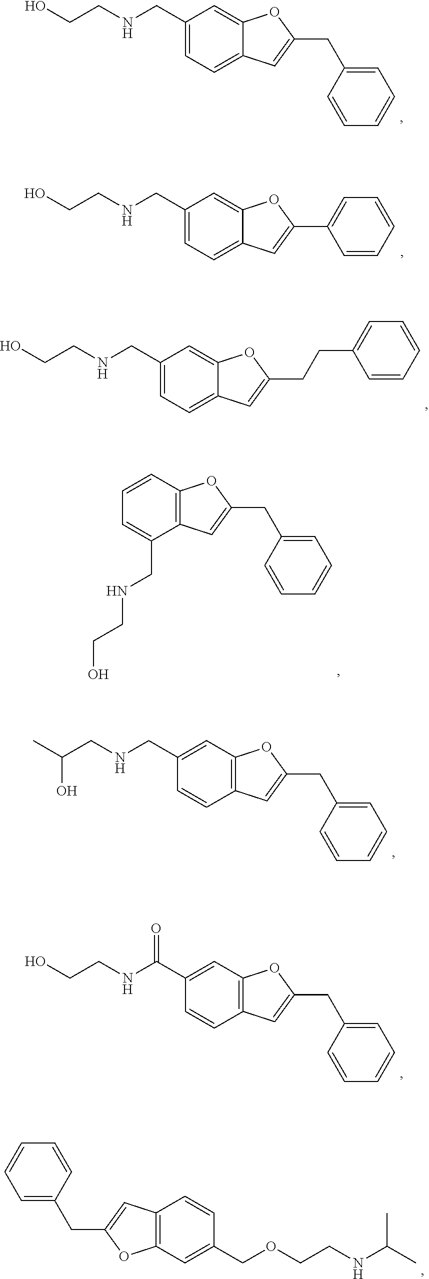 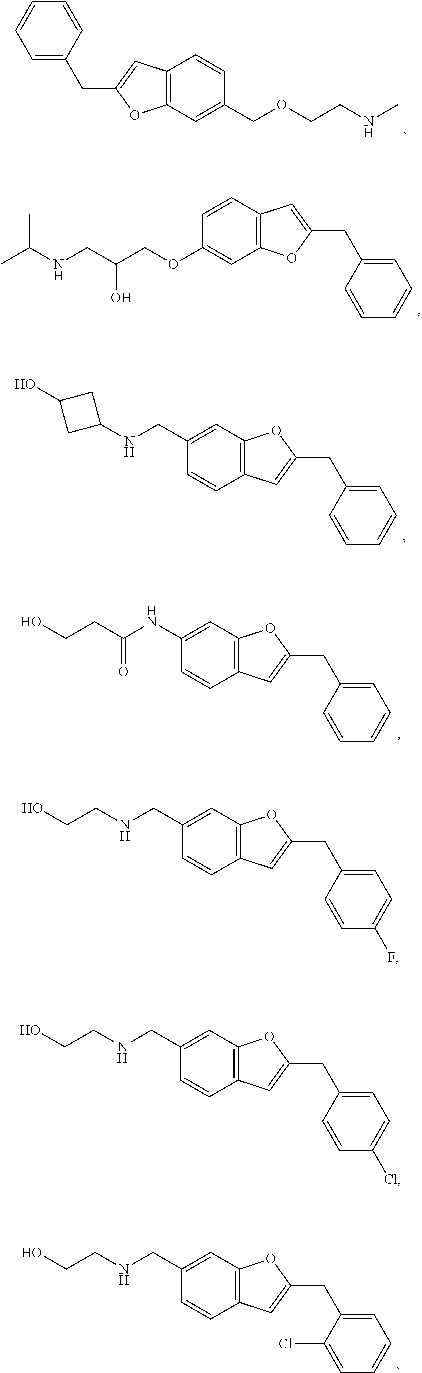 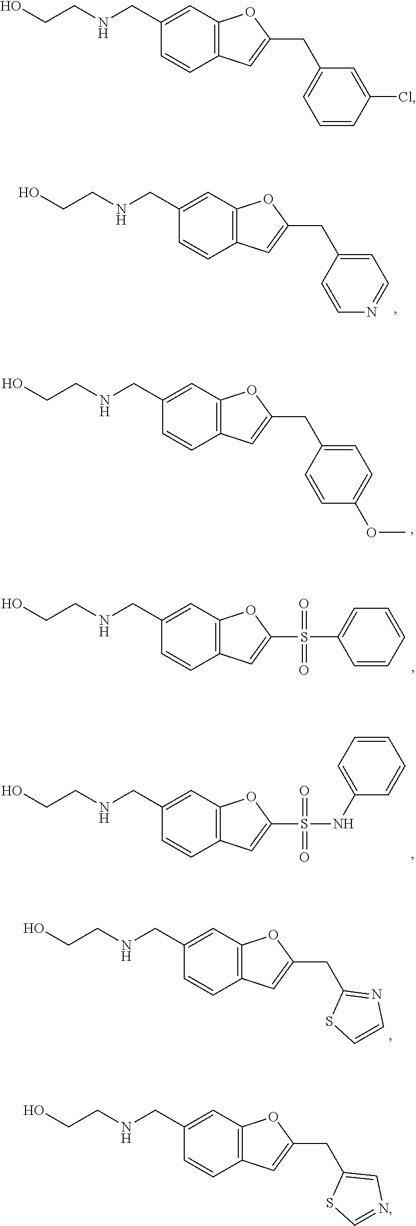 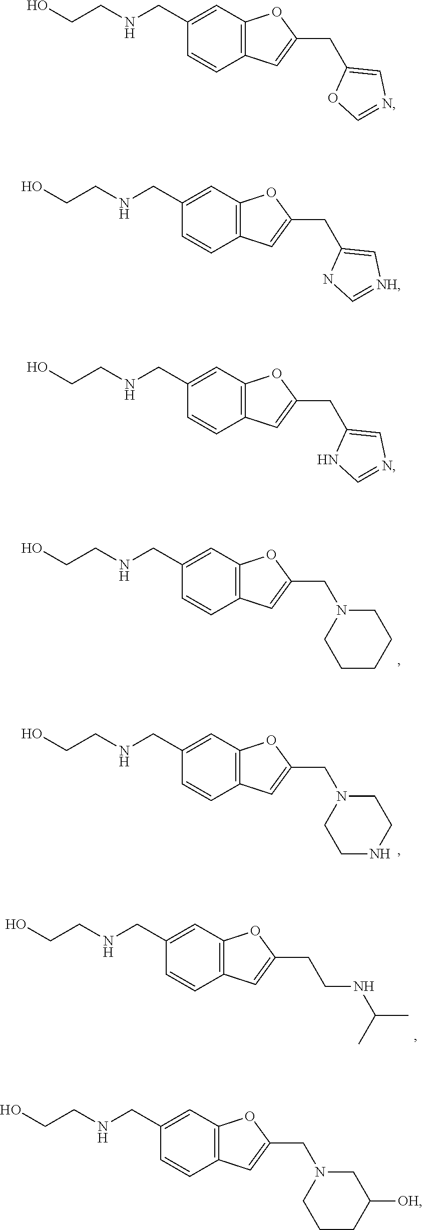 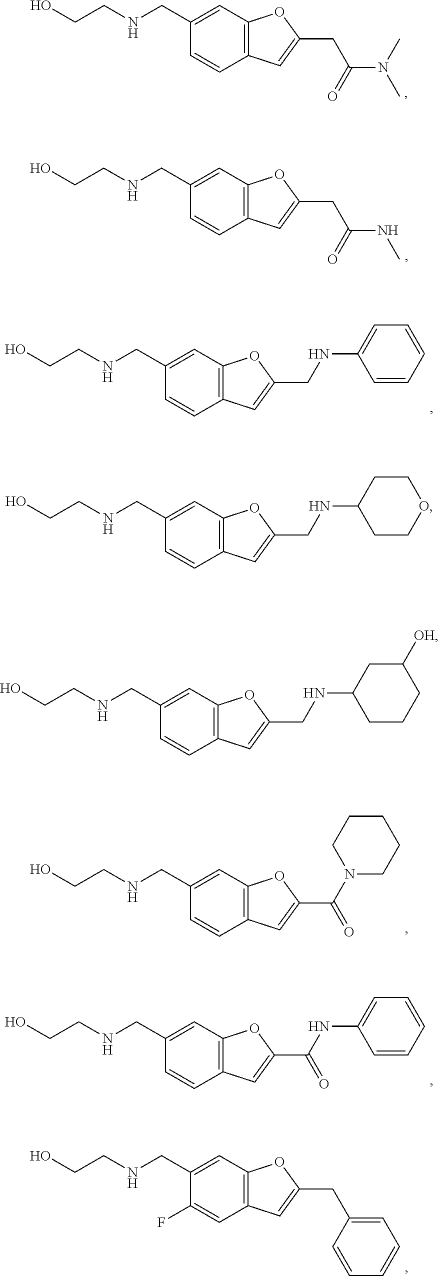 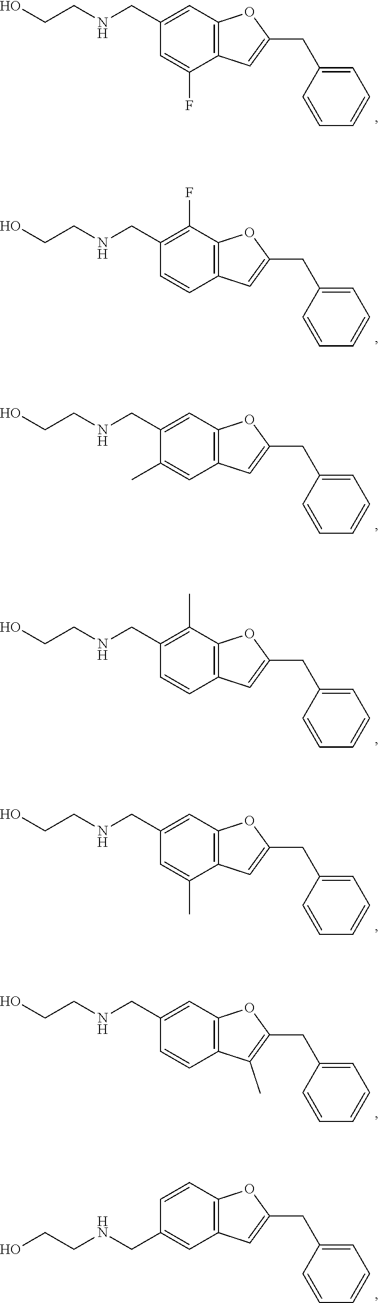 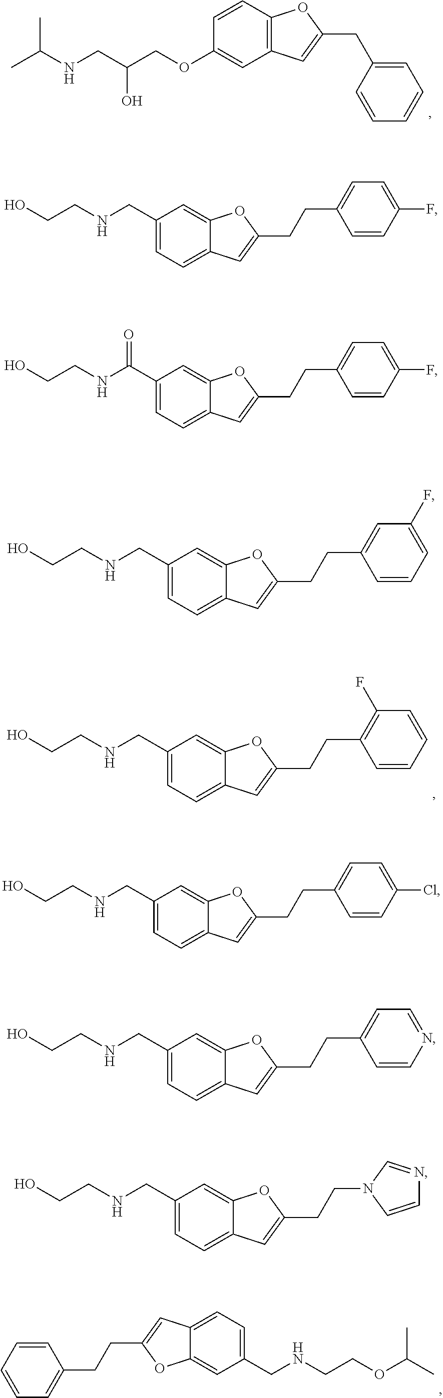 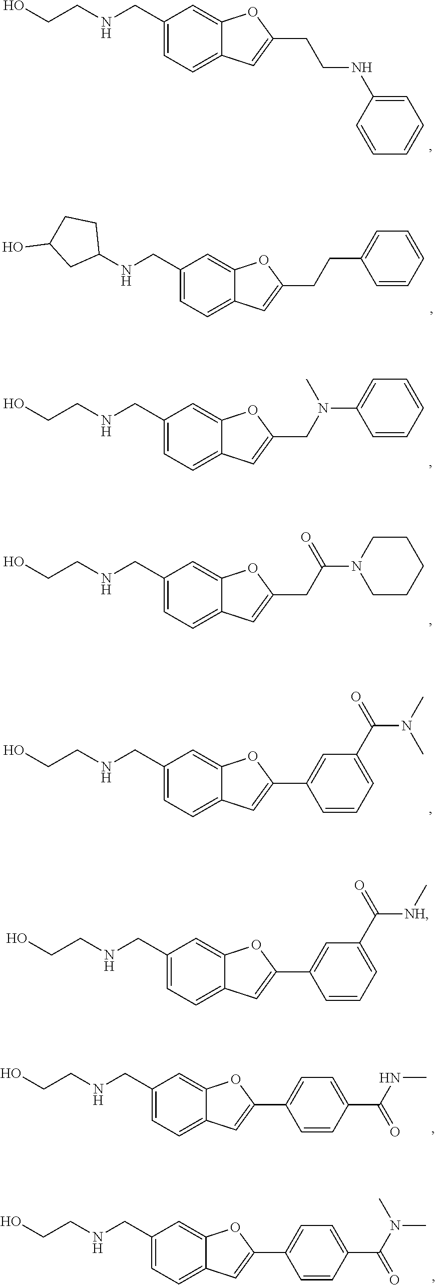 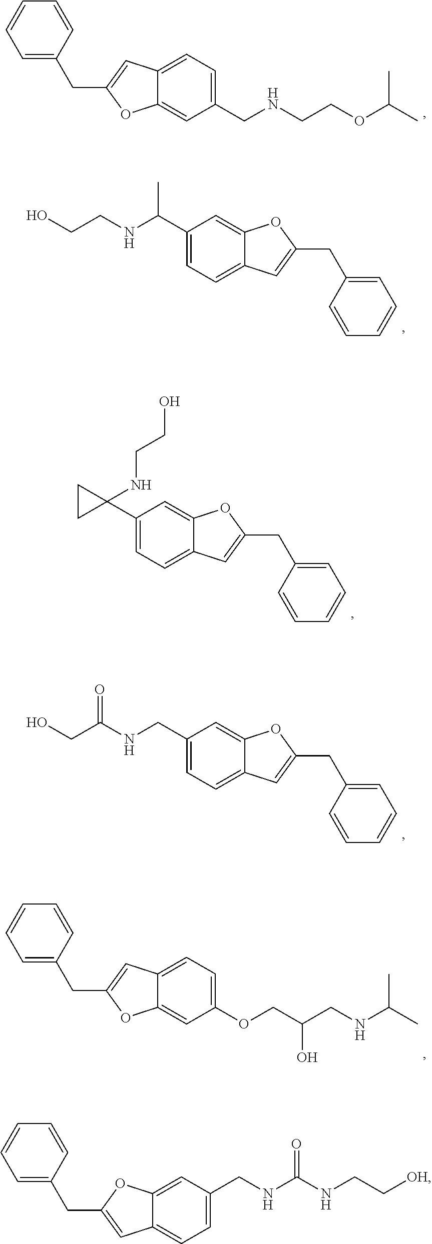 [SMILES: CC(C)OCCNCc1ccc2cc(Cc3ccccc3)oc2c1 ; CC(NCCO)c1ccc2cc(Cc3ccccc3)oc2c1 ; OCCNC1(c2ccc3cc(Cc4ccccc4)oc3c2)CC1 ; O=C(CO)NCc1ccc2cc(Cc3ccccc3)oc2c1 ; CC(C)NCC(O)COc1ccc2cc(Cc3ccccc3)oc2c1 ; O=C(NCCO)NCc1ccc2cc(Cc3ccccc3)oc2c1] 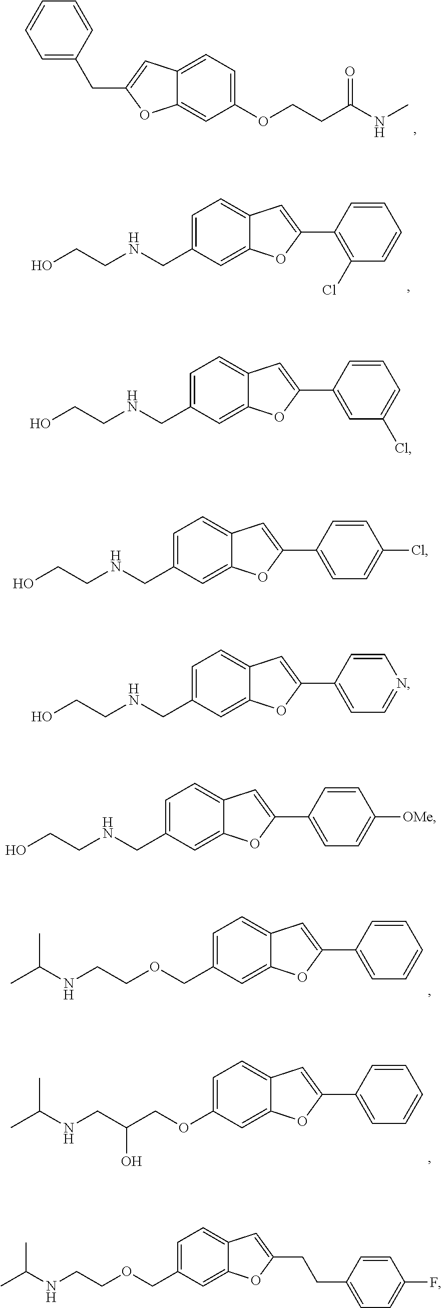 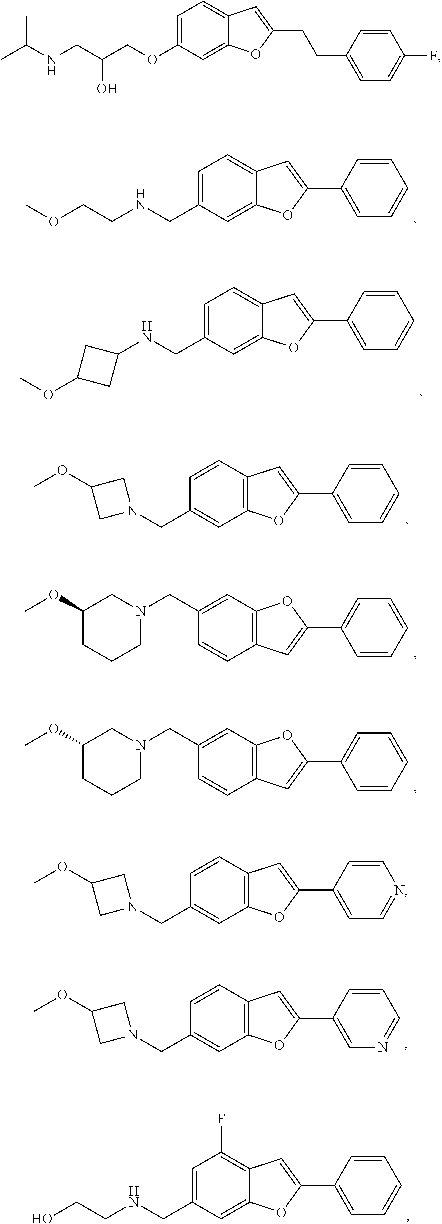 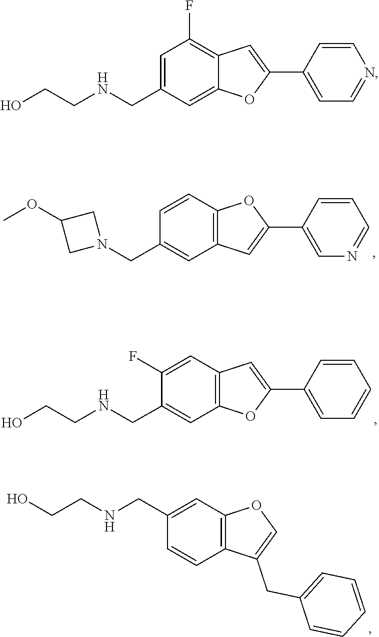 [SMILES: OCCNCc1cc(F)c2cc(-c3ccncc3)oc2c1 ; COC1CN(Cc2ccc3oc(-c4cccnc4)cc3c2)C1 ; OCCNCc1cc2oc(-c3ccccc3)cc2cc1F ; OCCNCc1ccc2c(Cc3ccccc3)coc2c1] 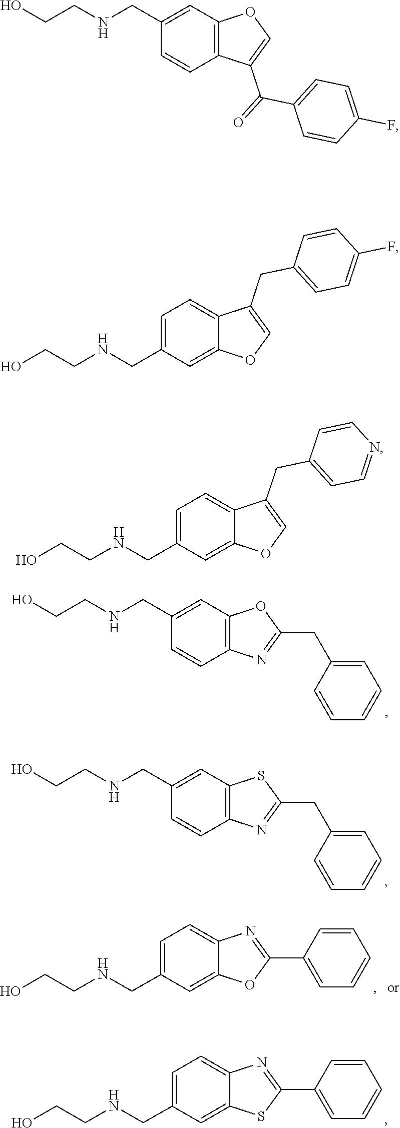
or a pharmaceutically acceptable salt, a stereoisomer, or deuterated form thereof.

96.-97. (canceled)

98. The compound of claim 15, wherein the compound of formula (II) is 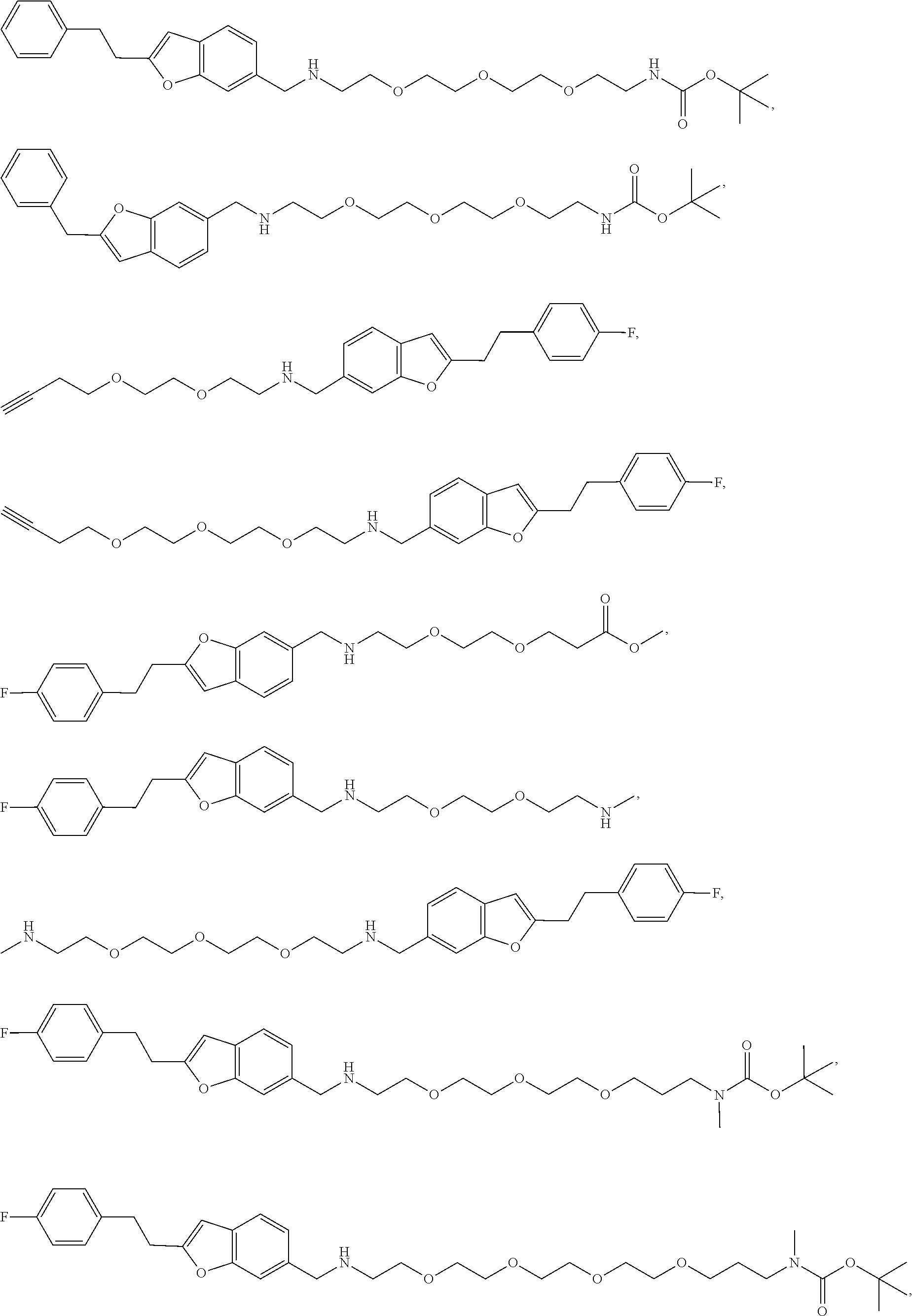 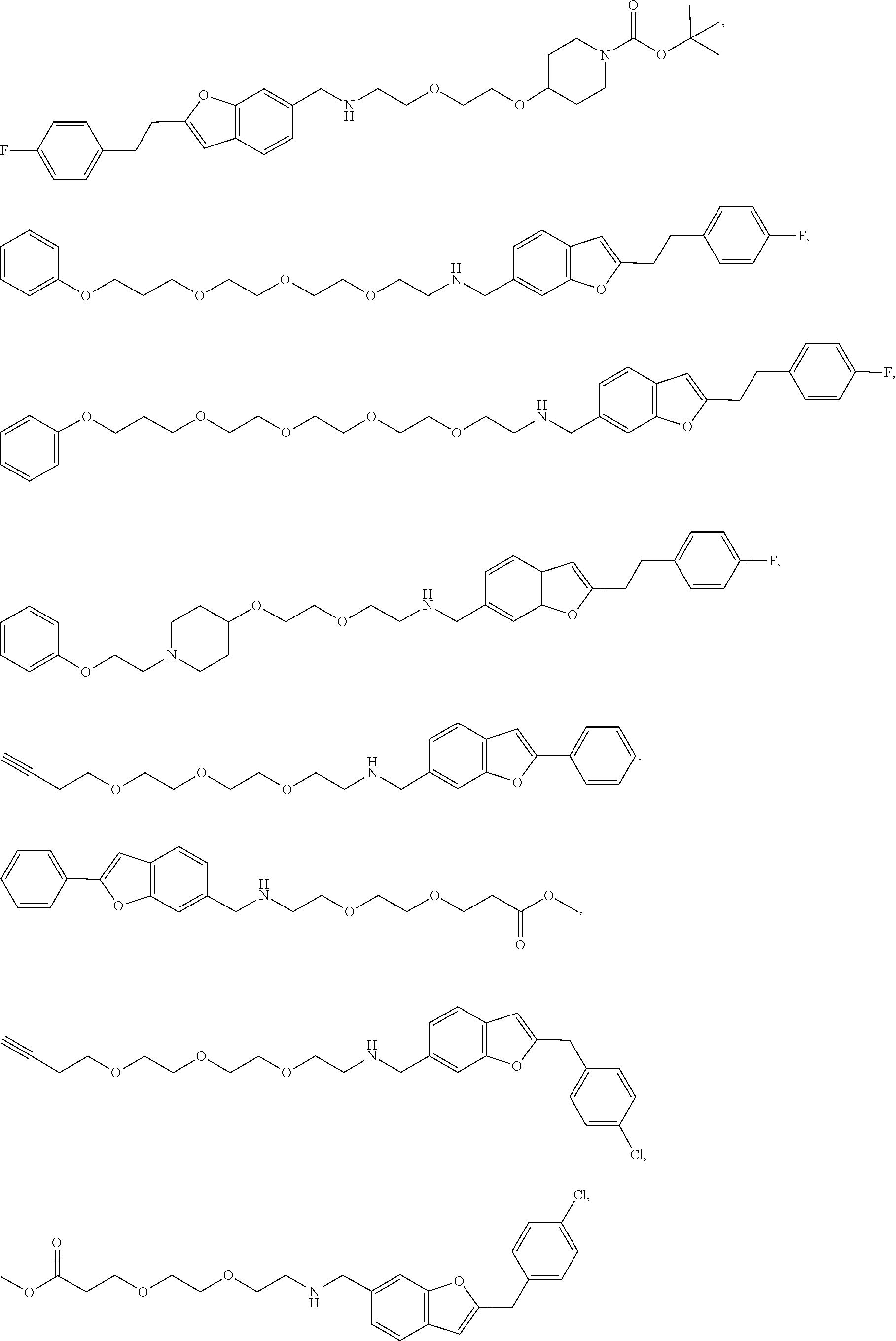 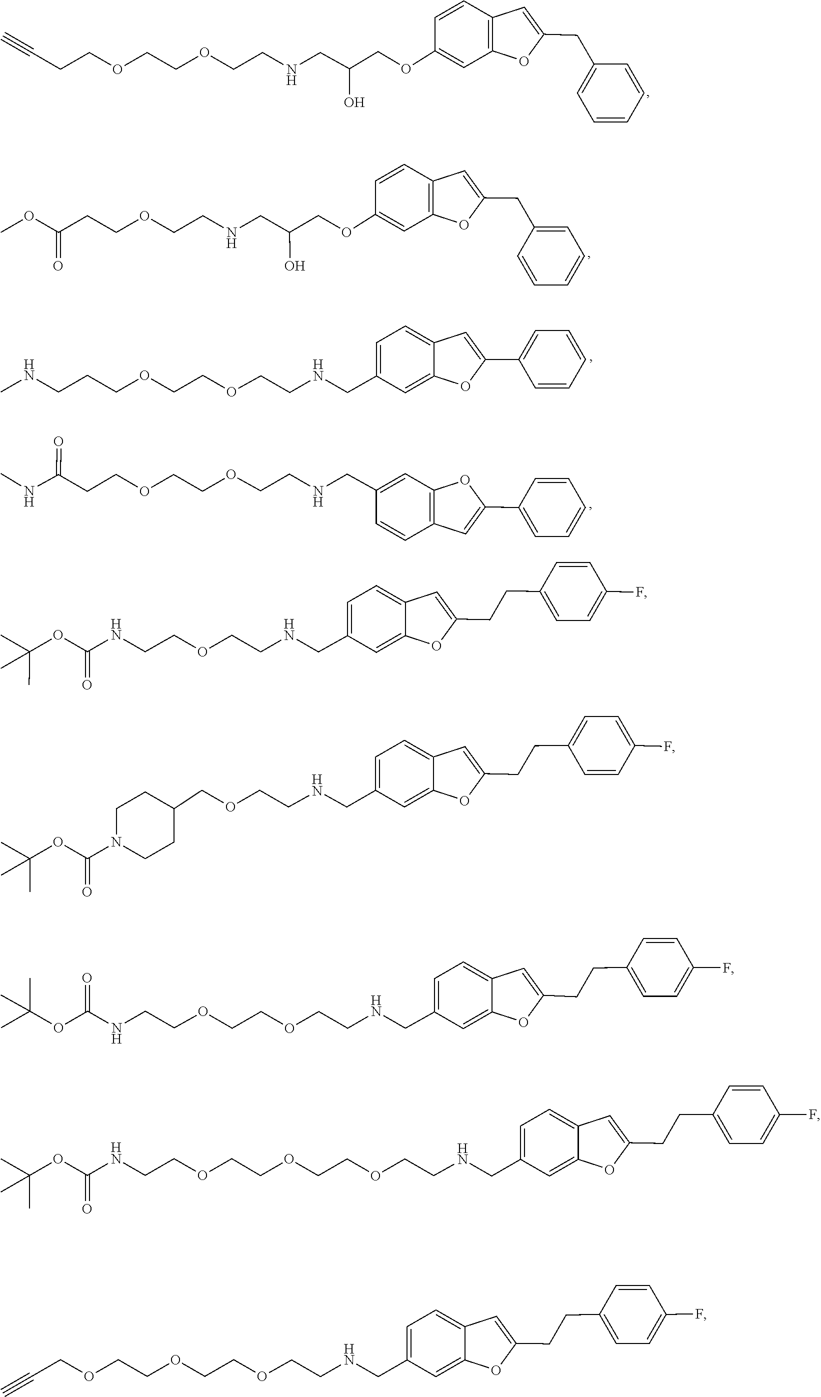 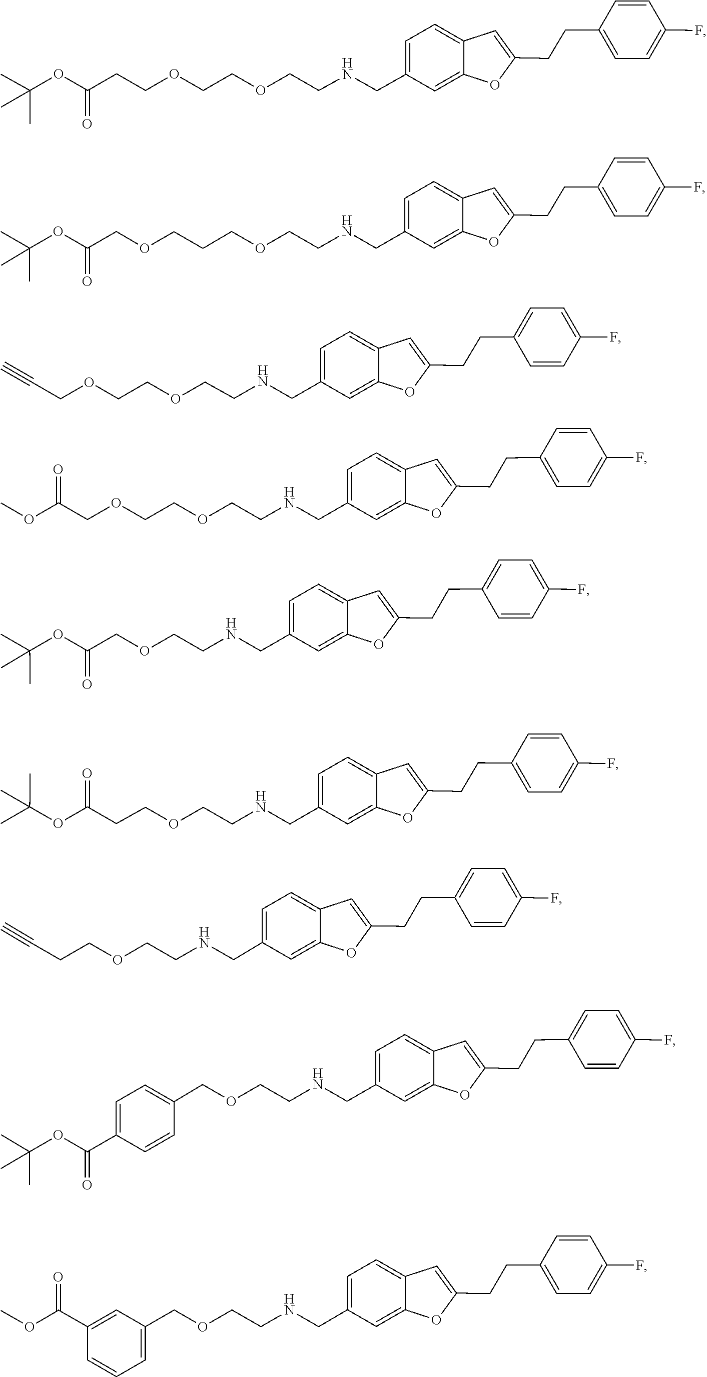 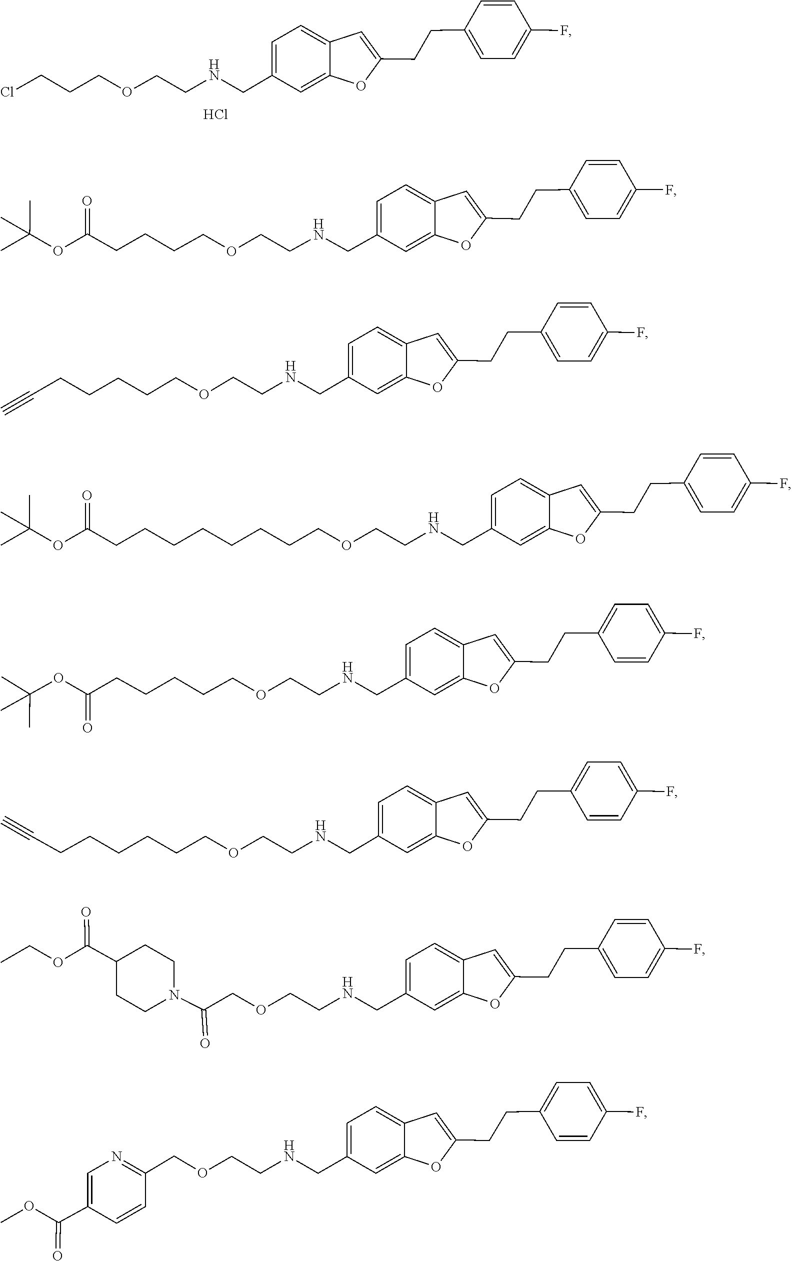 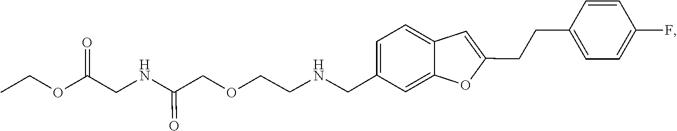 [SMILES: CCOC(=O)CNC(=O)COCCNCc1ccc2cc(CCc3ccc(F)cc3)oc2c1] 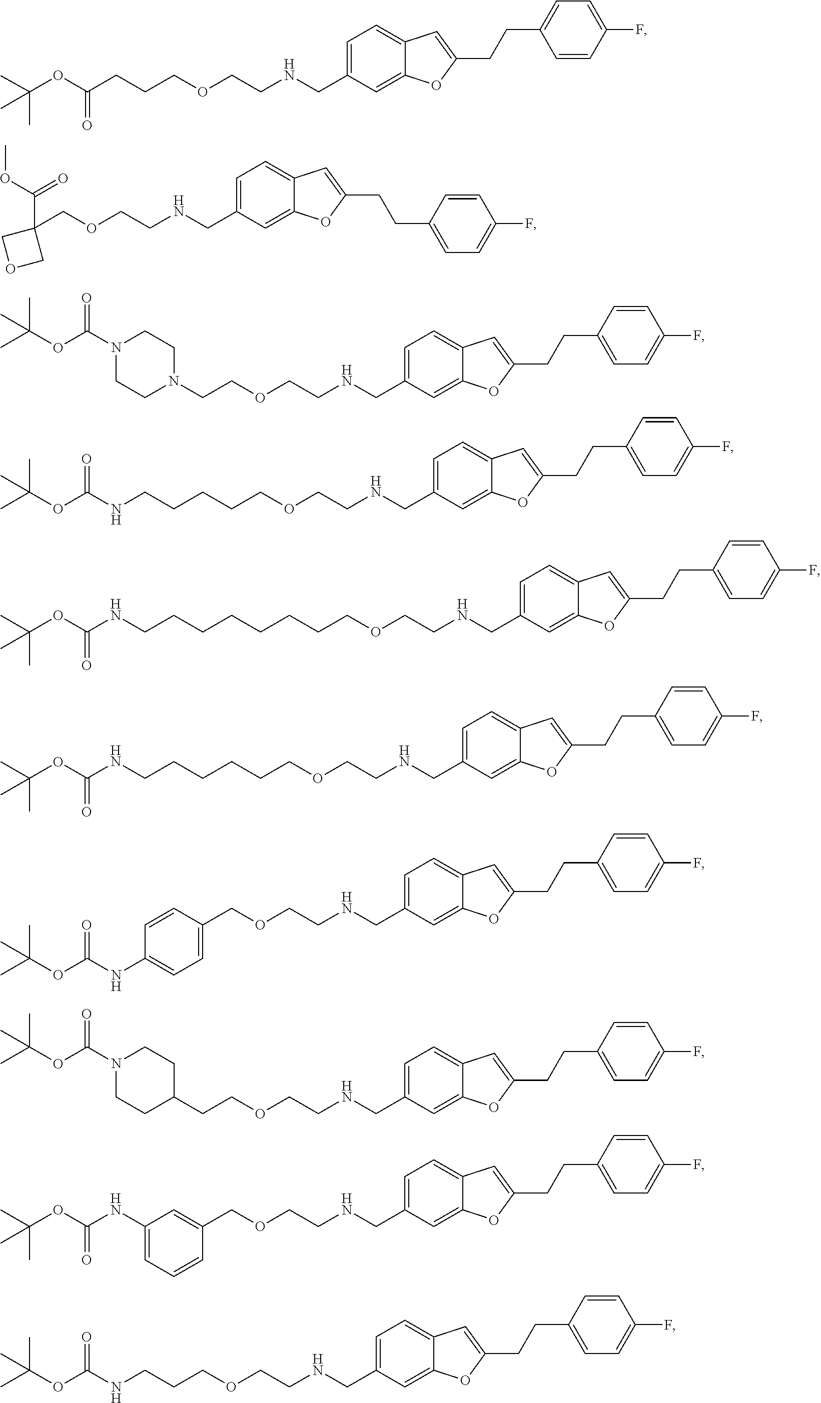 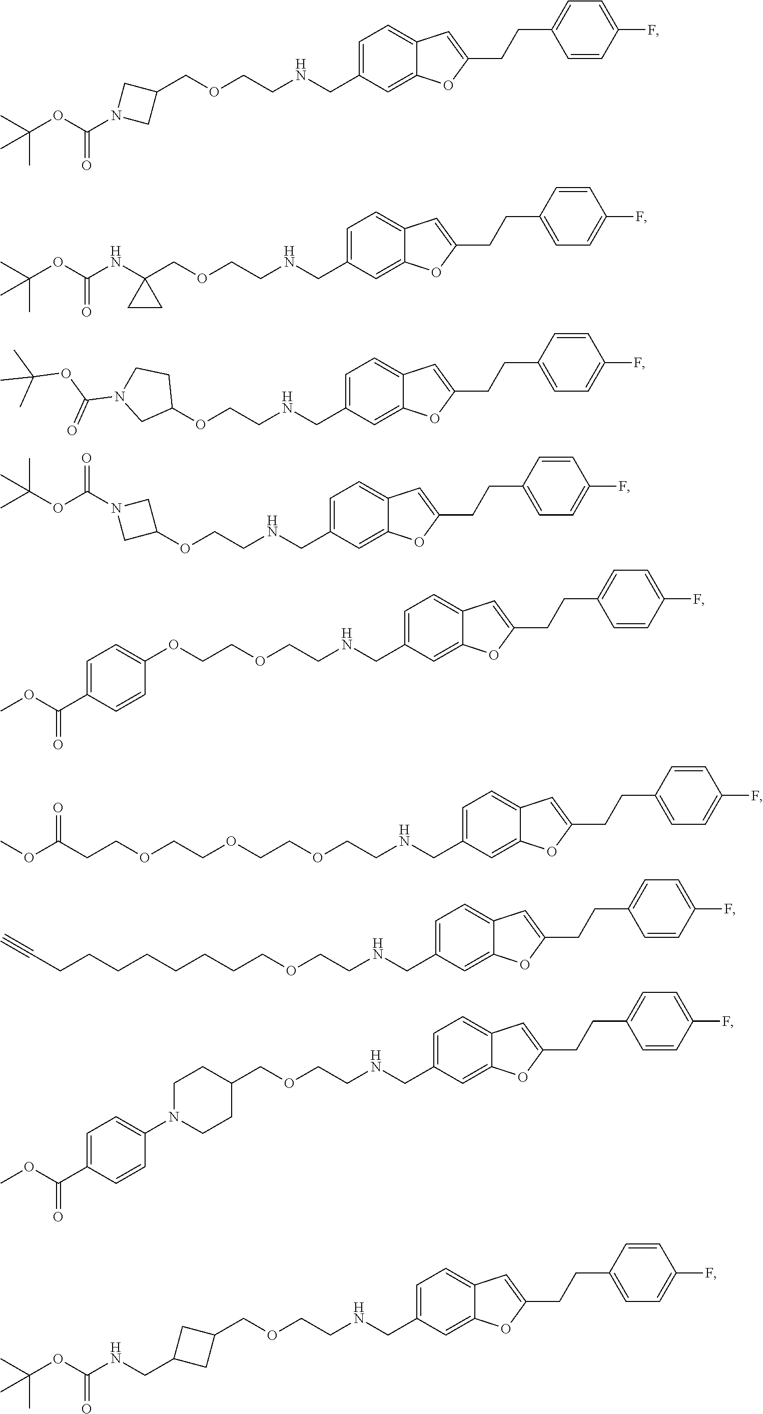 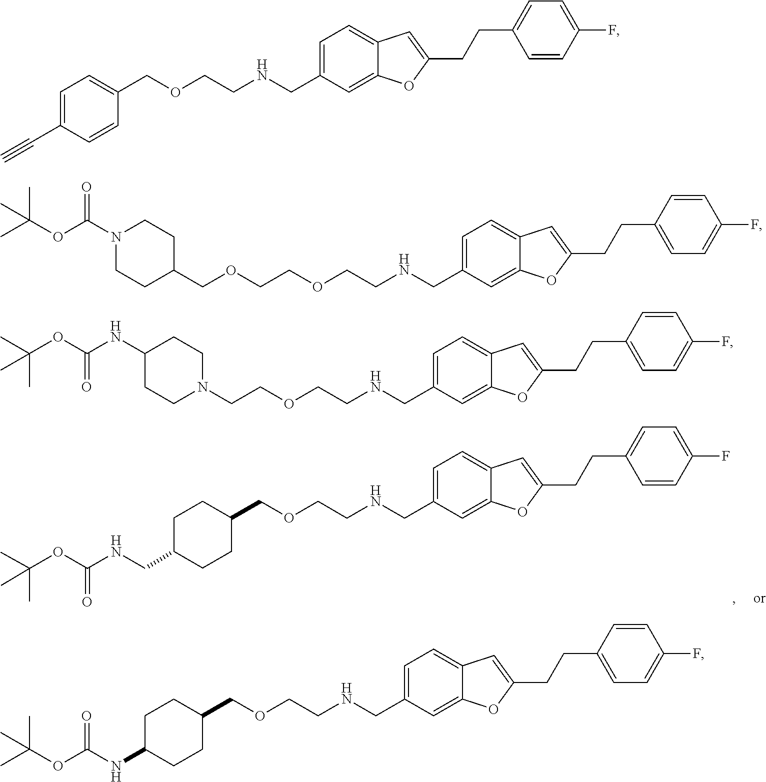
or a pharmaceutically acceptable salt, a stereoisomer, or deuterated form thereof.

99. The compound of claim 30, wherein the compound of formula (III) is 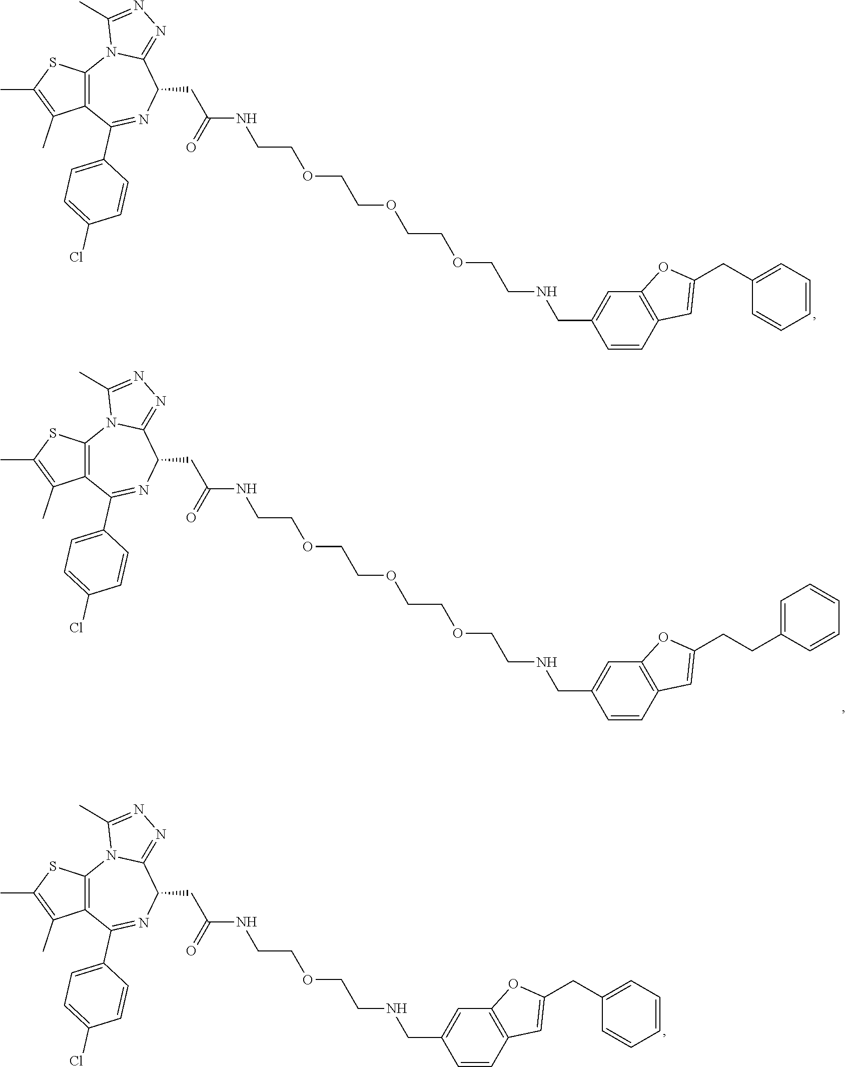 [SMILES: Cc1sc2c(c1C)C(c1ccc(Cl)cc1)=N[C@@H](CC(=O)NCCOCCOCCOCCNCc1ccc3cc(Cc4ccccc4)oc3c1)c1nnc(C)n1-2 ; Cc1sc2c(c1C)C(c1ccc(Cl)cc1)=N[C@@H](CC(=O)NCCOCCOCCOCCNCc1ccc3cc(CCc4ccccc4)oc3c1)c1nnc(C)n1-2 ; Cc1sc2c(c1C)C(c1ccc(Cl)cc1)=N[C@@H](CC(=O)NCCOCCNCc1ccc3cc(Cc4ccccc4)oc3c1)c1nnc(C)n1-2] 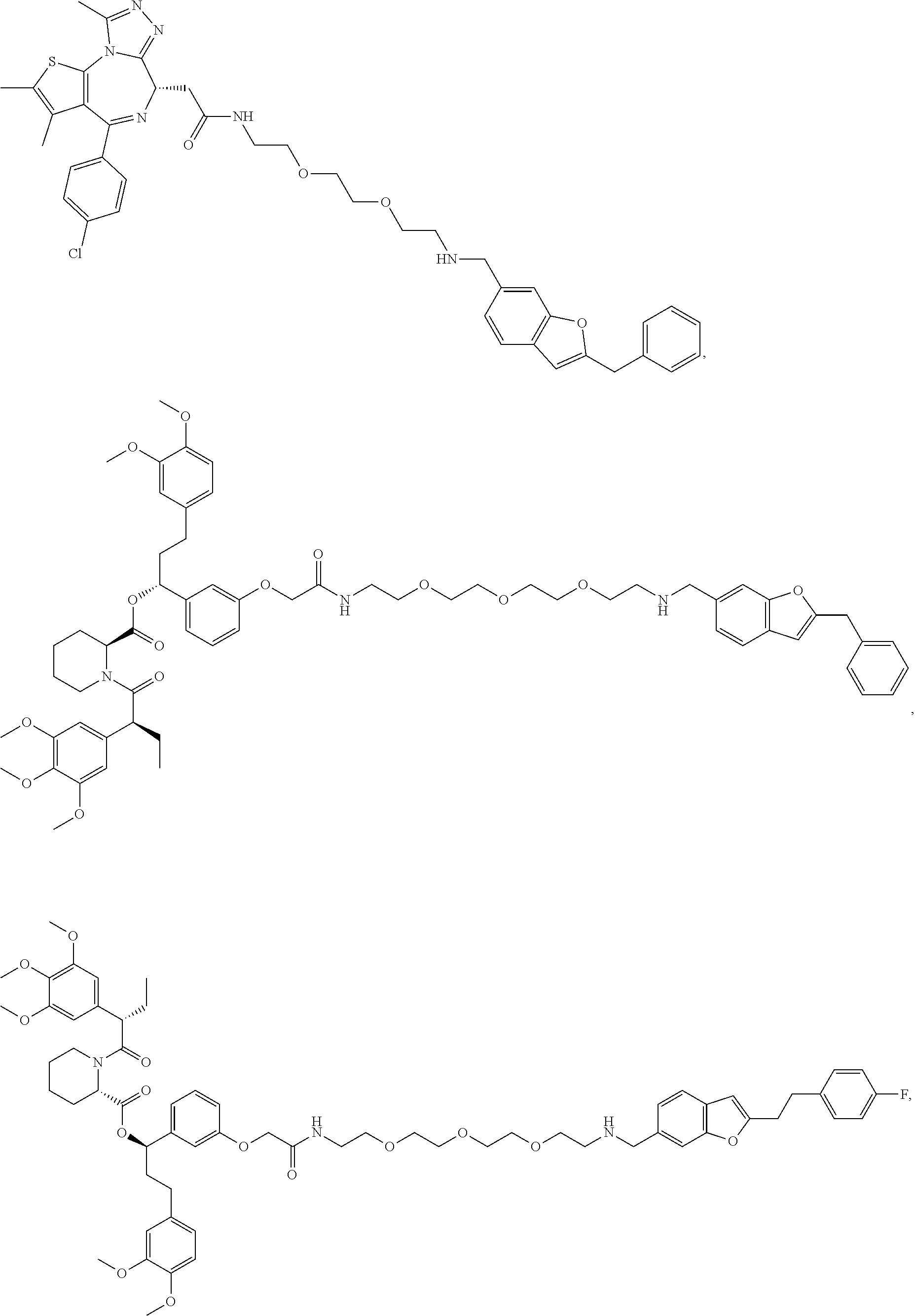 [SMILES: Cc1sc2c(c1C)C(c1ccc(Cl)cc1)=N[C@@H](CC(=O)NCCOCCOCCNCc1ccc3cc(Cc4ccccc4)oc3c1)c1nnc(C)n1-2 ; CC[C@H](C(=O)N1CCCC[C@H]1C(=O)O[C@H](CCc1ccc(OC)c(OC)c1)c1cccc(OCC(=O)NCCOCCOCCOCCNCc2ccc3cc(Cc4ccccc4)oc3c2)c1)c1cc(OC)c(OC)c(OC)c1 ; CC[C@H](C(=O)N1CCCC[C@H]1C(=O)O[C@H](CCc1ccc(OC)c(OC)c1)c1cccc(OCC(=O)NCCOCCOCCOCCNCc2ccc3cc(CCc4ccc(F)cc4)oc3c2)c1)c1cc(OC)c(OC)c(OC)c1] 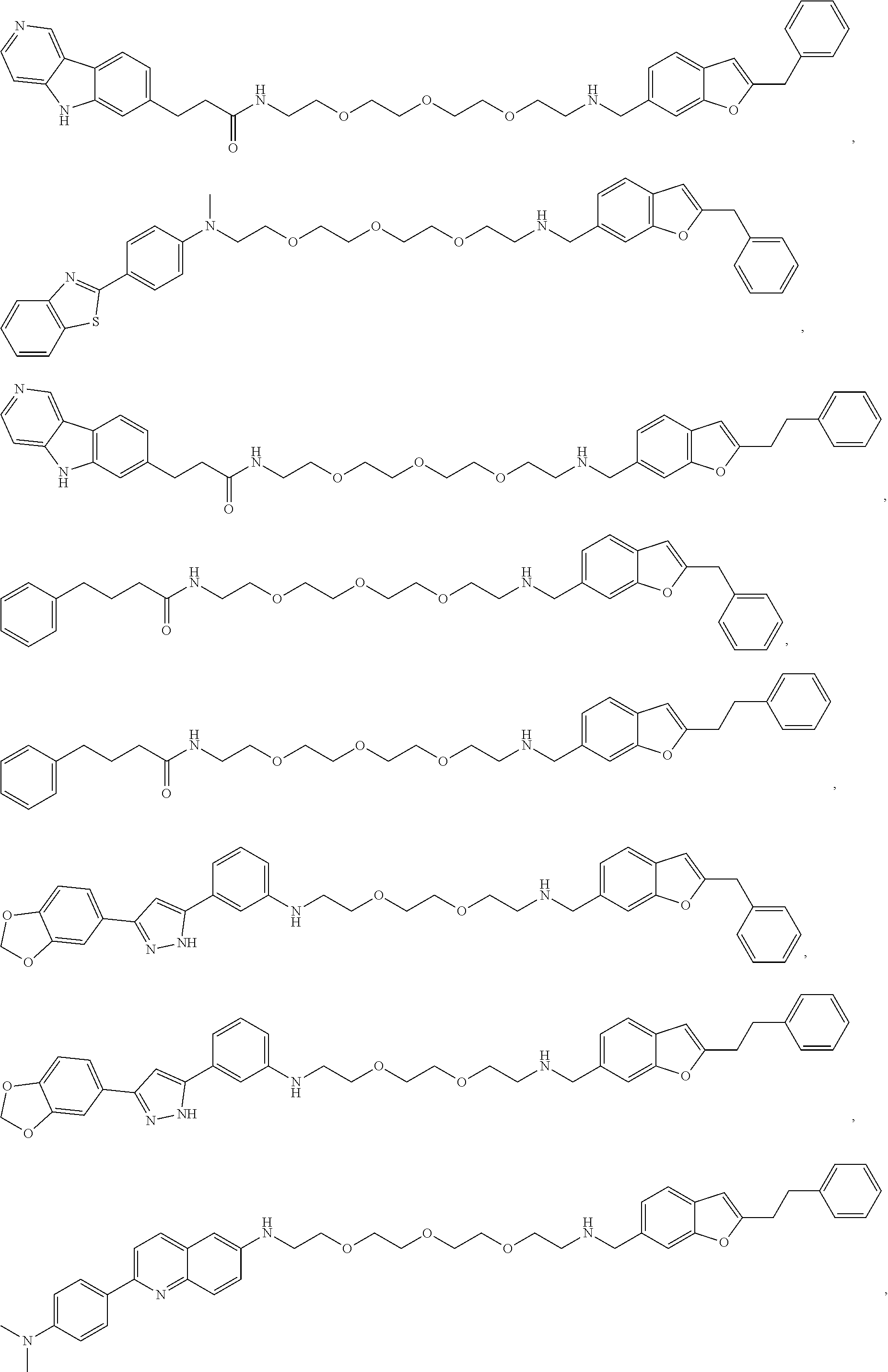 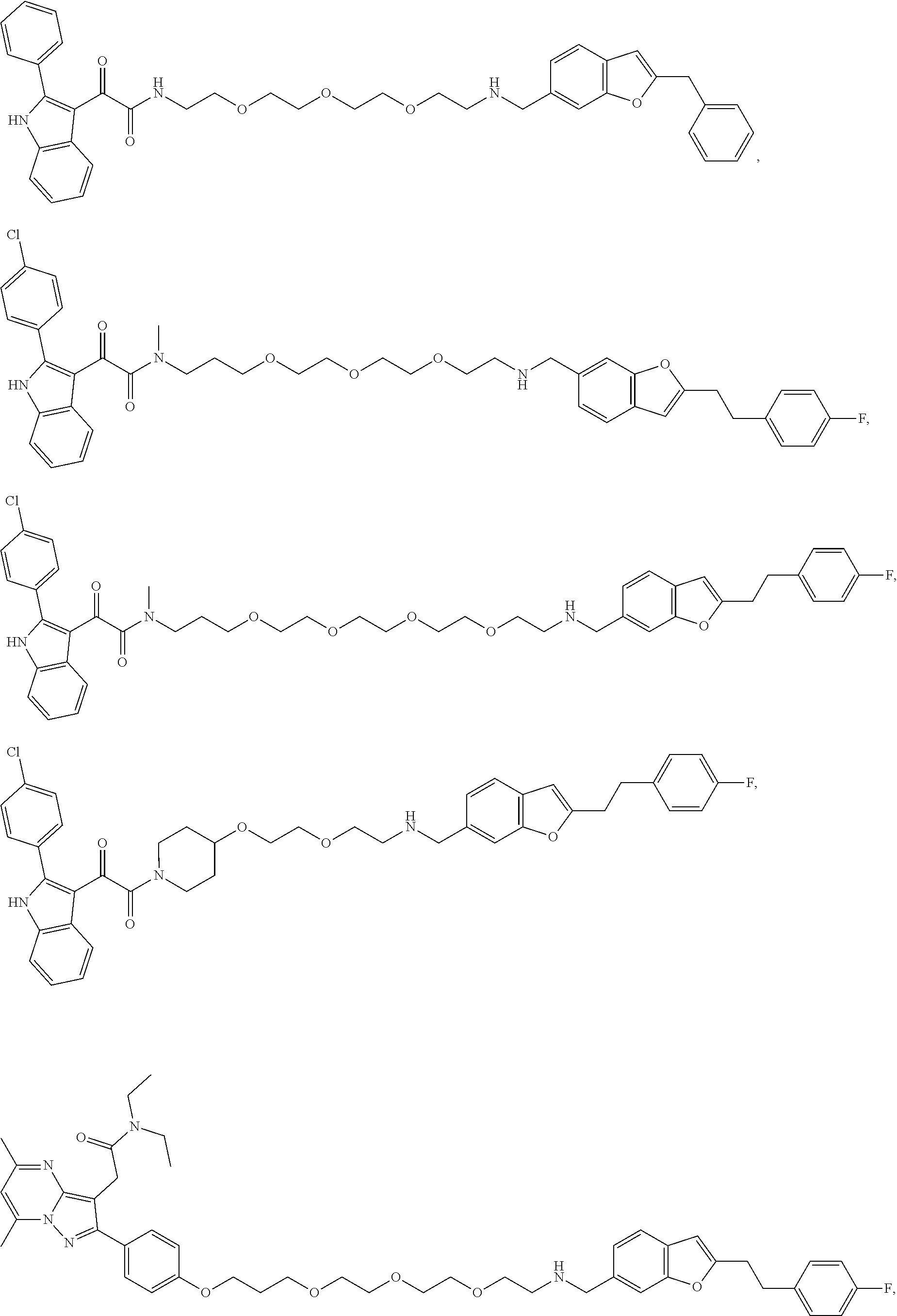 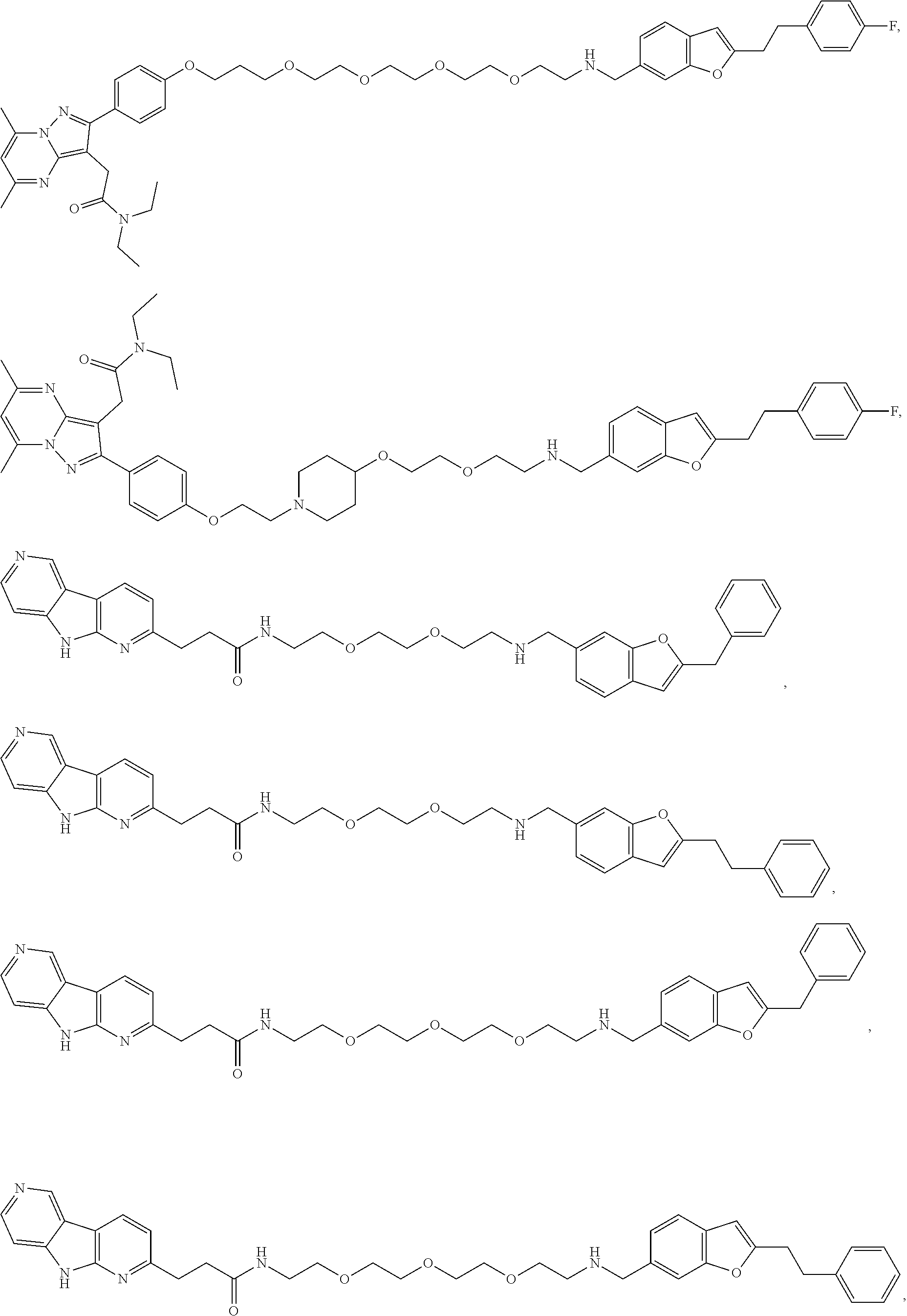 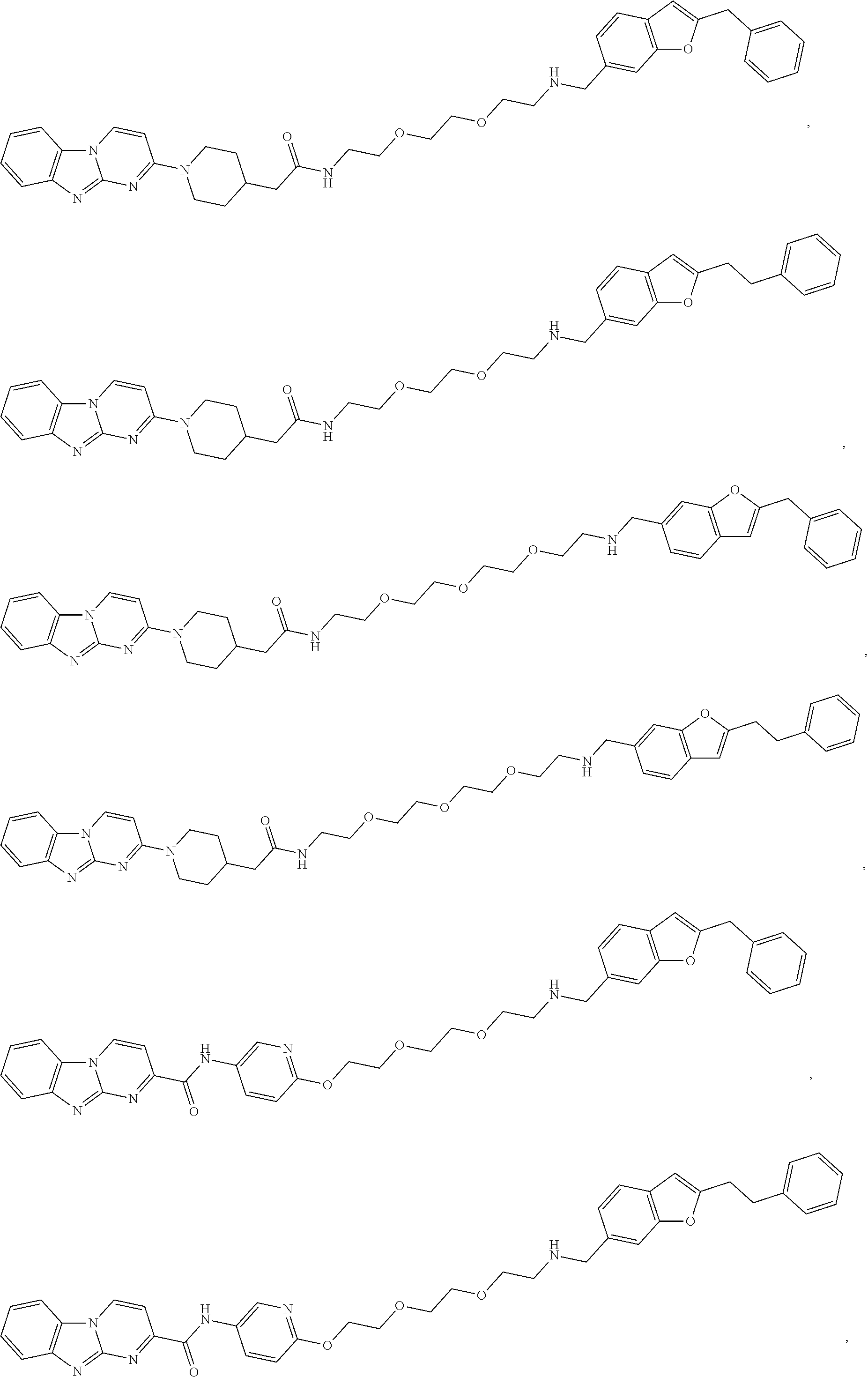 [SMILES: O=C(CC1CCN(c2ccn3c(n2)nc2ccccc23)CC1)NCCOCCOCCNCc1ccc2cc(Cc3ccccc3)oc2c1 ; O=C(CC1CCN(c2ccn3c(n2)nc2ccccc23)CC1)NCCOCCOCCNCc1ccc2cc(CCc3ccccc3)oc2c1 ; O=C(CC1CCN(c2ccn3c(n2)nc2ccccc23)CC1)NCCOCCOCCOCCNCc1ccc2cc(Cc3ccccc3)oc2c1 ; O=C(CC1CCN(c2ccn3c(n2)nc2ccccc23)CC1)NCCOCCOCCOCCNCc1ccc2cc(CCc3ccccc3)oc2c1 ; O=C(Nc1ccc(OCCOCCOCCNCc2ccc3cc(Cc4ccccc4)oc3c2)nc1)c1ccn2c(n1)nc1ccccc12 ; O=C(Nc1ccc(OCCOCCOCCNCc2ccc3cc(CCc4ccccc4)oc3c2)nc1)c1ccn2c(n1)nc1ccccc12] 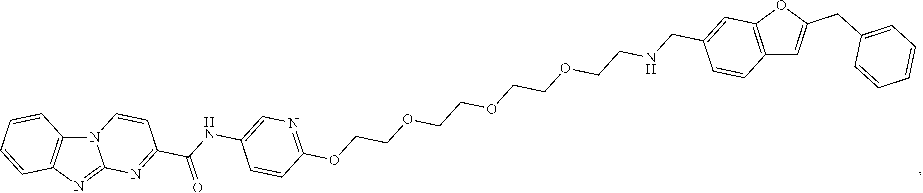 [SMILES: O=C(Nc1ccc(OCCOCCOCCOCCNCc2ccc3cc(Cc4ccccc4)oc3c2)nc1)c1ccn2c(n1)nc1ccccc12]
or a pharmaceutically acceptable salt, or deuterated form thereof.

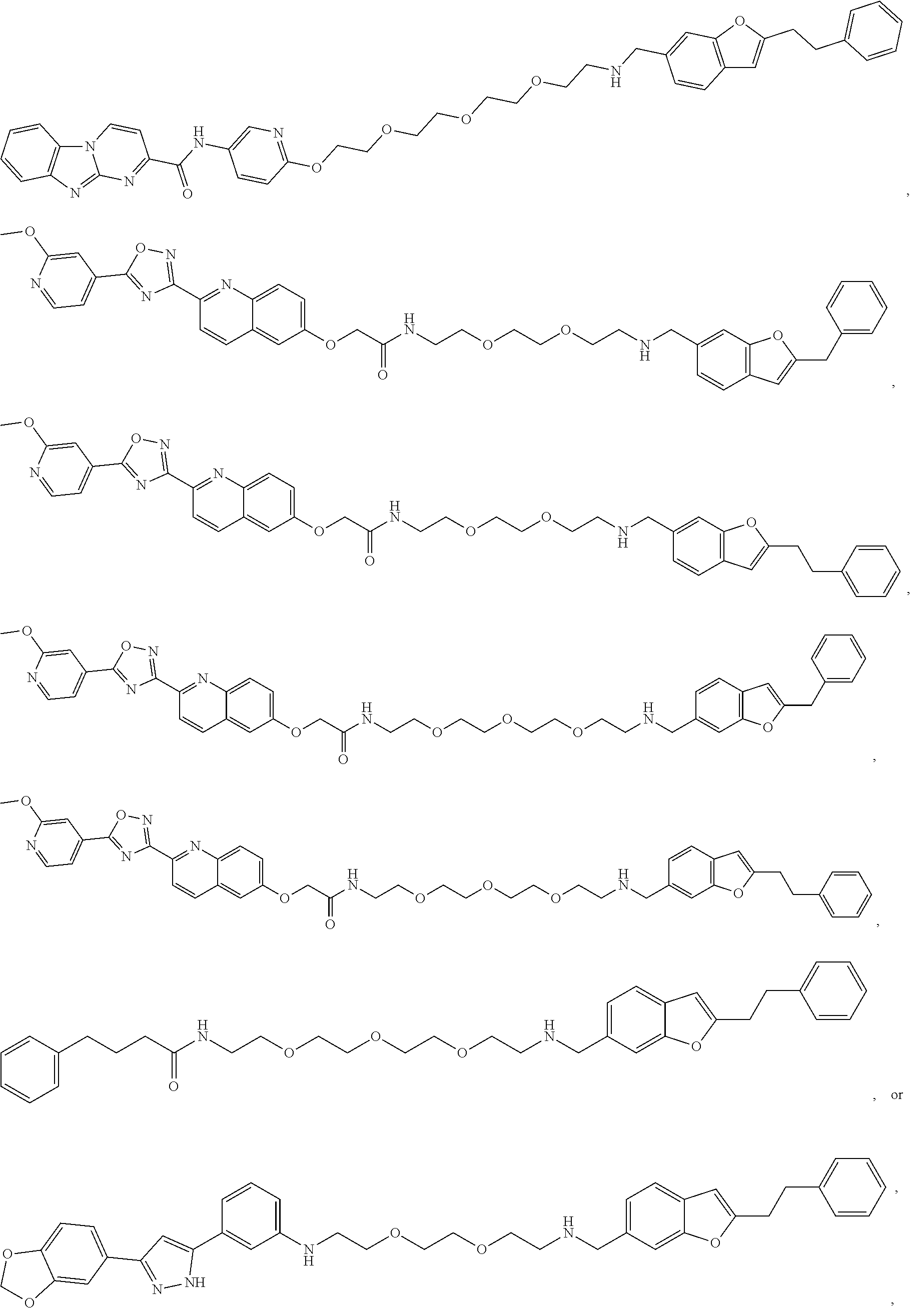

100.-106. (canceled)

107. A method of degrading a target protein in a subject in need thereof, comprising administering a compound of claim 1.

108.-110. (canceled)
